(19) United States
(12) Patent Application Publication
(10) Pub. No.: US 2023/0122079 A1
(43) Pub. Date: Apr. 20, 2023

(54) MASKED IL12 FUSION PROTEINS AND METHODS OF USE THEREOF

(71) Applicant: Zymeworks Inc., Vancouver (CA)

(72) Inventors: Ryan Blackler, Vancouver (CA); Gesa Volkers, Vancouver (CA); David Douda, Vancouver (CA); Thomas Spreter Von Kreudenstein, Vancouver (CA); Genevieve Desjardins, Vancouver (CA); Nicole Afacan, Vancouver (CA)

(21) Appl. No.: 17/914,261

(22) PCT Filed: Mar. 23, 2021
(86) PCT No.: PCT/CA2021/050383

§ 371 Date: Sep. 23, 2022

Related U.S. Application Data
(60) Provisional application No. 63/146,567, filed on Feb. 5, 2021.
(60) Provisional application No. 62/993,334, filed on Mar. 23, 2020.

(51) Int. Cl. C07K 14/54; C07K 14/715; A61P 35/00; C07K 16/24

(52) U.S. Cl. CPC C07K 14/5434; C07K 14/7155; A61P 35/00; C07K 16/244; C07K 2319/30; C07K 2319/50; C07K 2317/622; A61K 38/00

(57) ABSTRACT

The present disclosure relates to masked IL12 fusion proteins, compositions comprising the same and methods of using the compositions for the treatment of a variety of diseases including cancer.

### TECHNICAL FIELD

The present disclosure relates to masked IL12 fusion proteins, compositions comprising the same and methods of using the compositions for the treatment of a variety of diseases including cancer.

### BACKGROUND

Interleukin 12 (IL12) was the first recognized member of a family of heterodimeric cytokines that includes IL12, IL23, IL27, IL35, and IL39. IL12 and IL23 are pro-inflammatory cytokines important for development of T helper 1 (Th-1) and T helper 17 (Th-17) T cell subsets, while IL27 and IL35 are potent inhibitory cytokines. IL39 is an important cytokine in regulating innate and/or adaptive immune response. L12 can directly enhance the activity of effector CD4 and CD8 T cells as well as natural killer (NK) and NK T cells.

Interleukin-12 (IL12) is a heterodimeric molecule composed of an alpha chain (the p35 subunit) and a beta chain (the p40 subunit) covalently linked by a disulfide bridge to form the biologically active 70 kDa dimer. IL23 is a member of IL12 cytokine family and is also composed of two subunits: the p40 subunit that it shares with IL12 and p19. The IL12 receptor, or receptor complex, is composed of IL12Rβ1 and IL12Rβ2. The IL23 receptor complex (IL23R) consists of an IL23R subunit in complex with an IL12Rβ1 subunit, which is a common subunit for the IL12 receptor and interacts with Tyrosine kinase 2 (Tyk2). The IL23R is mainly expressed on immune cells, in particular T cells (e.g., Th17 and gamma delta T cells), macrophages, dendritic cells and NK cells (Duvallet et al., 2011). It has been recently shown that non-activated neutrophils express a basal amount of IL23R and that IL23R expression is increased upon cell activation (Chen et al., 2016).

Biologically, IL12 is an inflammatory cytokine that is produced in response to infection by a variety of cells of the immune system, including phagocytic cells, B cells and activated dendritic cells (Colombo and Trinchieri (2002), Cytokine and Growth Factor Reviews, 13: 155-168 and Hamza et al., “Interleukin-12 a Key Immunoregulatory Cytokine in Infection Applications” Int. J. Mol. Sci. 11; 789-806 (2010). IL12 plays an essential role in mediating the interaction of the innate and adaptive arms of the immune system, acting on T-cells and natural killer (NK) cells, enhancing the proliferation and activity of cytotoxic lymphocytes and the production of other inflammatory cytokines, especially interferon-gamma (IFN-gamma).

IL12 has been tested in human clinical trials as an immunotherapeutic agent for the treatment of a wide variety of cancers (Atkins et al. (1997), Clin. Cancer Res., 3: 409-17; Gollob et al. (2000), Clin. Cancer Res., 6: 1678-92; Hurteau et al. (2001), Gynecol. Oncol., 82: 7-10; and Youssoufian, et al. (2013) Surgical Oncology Clinics of North America, 22(4): 885-901), including renal, colon, and ovarian cancer, melanoma and T-cell lymphoma, and as an adjuvant for cancer vaccines (Lee et al. (2001), J. Clin. Oncol. 19: 3836-47). However, IL12 is toxic when administered systemically as a recombinant protein. Trinchieri, Adv. Immunol. 1998; 70:83-243. In order to maximize the anti-tumoral effect of IL12 while minimizing its systemic toxicity, IL12 gene therapy approaches have been proposed to allow production of the cytokine at the tumor site, thereby achieving high local levels of IL12 with low serum concentration. Qian et al., Cell Research (2006) 16: 182-188; US Patent Publication 20130195800.

Since IL12 is a heterodimeric molecule composed of an alpha chain (the p35 subunit) and a beta chain (the p40 subunit), the simultaneous expression of the two subunits is necessary for the production of the biologically active heterodimer. Recombinant IL12 expression has been achieved using bicistronic vectors containing the p40 and p35 subunits separated by an IRES (internal ribosome entry site) sequence to allow independent expression of both subunits from a single vector. However, use of IRES sequences can impair protein expression. Mizuguchi et al., Mol Ther (2000); 1: 376-382. Moreover, unequal expression of the p40 and p35 subunits can lead to the formation of homodimeric proteins (e.g., p40-p40) which can have inhibitory effects on IL12 signaling. Gillessen et al. Eur. J. Immunol. 25(1):200-6 (1995).

As an alternative to bicistronic expression of the IL12 subunits, functional single chain IL12 fusion proteins have been produced by joining the p40 and p35 subunits with (Gly4Ser)3 or Gly6Ser linkers. Lieschke et al., (1997), Nature Biotechnology 15, 35-40; Lode et al., (1998), PNAS 95, 2475-2480. (These forms of p40-linker-p35 or p35-linker-p40 IL12 configurations may be referred to herein as “single chain IL12 (scIL12)”).

Human IL12 p70 (i.e., dimer of p35 and p40) has a reported in vivo half-life of 5-19 hours which, when administered as a therapeutic compound, can result in significant systemic toxicity. See e.g., Car et al. “The Toxicology of Interleukin-12: A Review” Toxicologic Path. 27:1, 58-63 (1999); Robertson et al. “Immunological Effects of Interleukin 12 Administered by Bolus Intravenous Injection to Patients with Cancer” Clin. Cancer Res. 5:9-16 (1999); Atkins et al. “Phase I Evaluation of Intravenous Recombinant Human Interleukin 12 in Patients with Advance Malignancies” Clin. Cancer Res. 3:409-417 (1997). Preclinical studies in murine tumor treatment models demonstrate powerful antitumor effects following the systemic administration of IL12. In humans, however, attempts to systemically administer recombinant IL12 resulted in significant toxicities including patient deaths and limited efficacy. Thus, there remains a need in the art for improved therapeutic control of in vivo delivered forms of IL12.

### SUMMARY

One aspect of the present disclosure provides a masked interleukin 12 (IL12) fusion protein, comprising: a) an Fc domain comprising a first Fc polypeptide and a second Fc polypeptide; b) a masking moiety (MM); and c) an IL12 polypeptide; wherein the masking moiety is fused to the first Fc polypeptide by a first linker; and optionally, wherein the masking moiety further comprises a second linker; wherein the IL12 polypeptide is fused to the second Fc polypeptide by a third linker; wherein at least one of the first, second or third linkers is protease cleavable; and wherein the IL12 activity of the masked IL12 fusion protein is attenuated as compared to the IL12 activity of the IL12 containing polypeptide released after cleavage of the at least one protease cleavable linker. In some embodiments of the masked IL12 fusion proteins, the first linker is protease cleavable and optionally, the second linker is protease cleavable. In some embodiments of the masked IL12 fusion proteins, the first linker is optionally protease cleavable and the second linker is protease cleavable. In some embodiments of the masked IL12 fusion proteins, the third linker is protease cleavable and optionally, the first or second linker is protease cleavable, or both. In some embodiments of the masked IL12 fusion proteins, the first linker comprises a cleavage sequence selected from the group consisting of the cleavage sites listed in Table 3 and Table 24. In some embodiments of the masked IL12 fusion proteins herein, the first linker comprises a cleavage sequence having the amino acid sequence MSGRSANA (SEQ ID NO:10). In some embodiments, the protease cleavable linker is cleaved by a protease selected from the group consisting of a matrix metalloproteinase (MMP), a matriptase, a cathepsin, a kallikrein, a caspase, a serine protease, thrombin, chymase, carboxypeptidase A, tryptase, cathepsin G, cathepsin L, ADAM metalloproteinase, and an elastase. In one embodiment, the first, second and third linkers are cleaved by the same protease.

In some embodiments of the masked IL12 fusion proteins herein, the masking moiety is a single-chain Fv (scFv) antibody fragment, an IL12 receptor β2 subunit (IL12Rβ2) or an IL12-binding fragment thereof, or an IL12 receptor 131 subunit (IL12Rβ1) or an IL12-binding fragment thereof. In certain embodiments, the scFv comprises the VHCDR1-3 having the amino acid sequences set forth in SEQ ID NOS:13-15, respectively and the VLCDR1-3 having the amino acid sequence set forth in SEQ ID NOS: 16-18, respectively. In some embodiments, the scFv comprises a VH and VL comprising the amino acid sequence set forth in SEQ ID NOs:11 and 12, respectively; or a VH and VL comprising the amino acid sequence set forth in SEQ ID NOs:255 and 256, respectively. In some embodiments, the scFv comprises a variant of the VH having the amino acid sequence set forth in SEQ ID NO:11 wherein the variant is selected from the group consisting of H_Y32A; H_F27V; H_Y52AV; H_R52E; H_R52E_Y52AV; H_H95D; H_G96T; and H_H98A, according to Kabat numbering; and the VL having the amino acid sequence set forth in SEQ ID NO: 12. In some embodiments, the masking moiety is selected from an ECD of human IL12Rβ2, amino acids 24-321 of human Th12Rβ2 (IL12Rβ224-321), amino acids 24-124 of human IL12Rβ2 (IL12Rβ24-124), amino acids 24-240 of human IL12Rβ1 (IL12Rβ124-240) and an IL23R ECD.

In some embodiments of the masked IL12 fusion protein herein, the IL12 polypeptide comprises the amino acid sequence set forth in SEQ ID NO:22 or 23. In some embodiments, the IL12 polypeptide comprises the p40 polypeptide amino acid sequence set forth in SEQ ID NO:22 and the p35 IL12 polypeptide is non-covalently bound to the p40 polypeptide. In some embodiments, the IL12 polypeptide comprises the p35 polypeptide amino acid sequence set forth in SEQ ID NO:23 and the p40 IL12 polypeptide is non-covalently bound to the p40 polypeptide.

In some embodiments of the masked IL12 fusion proteins herein, the IL12 is a single chain IL12 polypeptide selected from a single chain IL12 polypeptide having the orientation p35-linker-p40 or p40-linker-p35. In some embodiments, the fusion protein is selected from variants 29243, 29244, 31277, 32039, 32042, 32045, 33507, 35425, 32041, 35436, 35437, 32862 and 32454. In some embodiments, the single chain IL12 polypeptide is a p40-linker-p35 polypeptide that is fused to the second Fc polypeptide at the p40 polypeptide. In some embodiments, the single chain IL12 polypeptide is a p35-linker-p40 polypeptide that is fused to the second Fc polypeptide at the p35 polypeptide. In some embodiments, the single chain IL12 polypeptide is fused to the c-terminal end of the second Fc polypeptide. In some embodiment of the masked IL12 fusion proteins, the single chain IL12 polypeptide is fused to the c-terminal end of the second Fc polypeptide and the masking moiety is fused to the c-terminal end of the first Fc polypeptide. In some embodiments, the single chain IL12 polypeptide is fused to the second Fc polypeptide and the third linker is protease cleavable. In some embodiments, the P40 domain of the IL12 polypeptide has been modified to be more resistant to proteolytic cleavage as compared to an unmodified P40 domain. In some embodiments, the masking moiety is a single-chain Fv (scFv) antibody fragment; and the IL12 fusion protein further comprises a second masking moiety comprising an additional scFv fused by a fourth linker to the p35 domain of the IL12 polypeptide. In some embodiments, the first and fourth linkers are protease cleavable. In some embodiments, the masking moiety comprises a first scFv fused to a second scFv by a fourth linker. In some embodiments, the first and fourth linkers are protease cleavable. In some embodiments, the masking moiety is in the following orientation: first Fc polypeptide-L1-VH-VL-L4-VH-VL; or first Fc polypeptide-L1-VH-VL-L4-VL-VH. In some embodiments, the first and fourth linkers are protease cleavable.

In some embodiment of the masked IL12 fusion proteins, the masking moiety comprises an IL12 receptor β2 subunit (IL12Rβ2) or an IL12-binding fragment thereof, and an IL12 receptor 131 subunit (IL12Rβ1) or an IL12-binding fragment thereof, fused by the second linker. In some embodiments, the masking moiety comprises an IL12Rβ2-Ig domain fused to the c-terminal end of the first Fc polypeptide and the IL12Rβ1 fused by the second linker to the c-terminal end of the IL12Rβ2-Ig domain. In some embodiments, the first and the second linker are protease cleavable. In some embodiments, the masking moiety is an IL12Rβ1 or an IL12-binding fragment thereof; and wherein the IL12 fusion protein further comprises a second masking moiety comprising an IL12Rβ2 or an IL12-binding fragment thereof fused by a fourth linker to the p35 domain of the IL12 polypeptide. In some embodiments, the first and the fourth linker are protease cleavable.

In some embodiments of the masked IL12 fusion proteins herein, the fusion protein further comprises a targeting domain. In some embodiments, the targeting domain specifically binds a tumor-associated antigen.

In some embodiments of the masked IL12 fusion proteins herein, the first Fc polypeptide comprises a first CH3 domain and the second Fc polypeptide comprises a second CH3 domain.

In some embodiments of the masked IL12 fusion proteins herein, the IL12 activity is determined by measuring relative cell abundance or cytokine production of a cell or a cell line that is sensitive to IL12. In some embodiments, the cell or cell line is selected from PBMC, CD8+ T cells, a CTLL-2 cell line and an NK cell line. In some embodiments, the IL12 activity is determined by measuring IFNγ release by CD8+ T cells. In some embodiments, the IL12 activity is determined by measuring the relative cell abundance of NK cells.

In some embodiments of the masked IL12 fusion proteins, the first CH3 domain or the second CH3 domain or both comprise an asymmetric amino acid modification wherein the first and second CH3 domain preferentially pair to form a heterodimer rather than a homodimer.

One aspect of the present disclosure provides a masked interleukin 12 (IL12) fusion protein, comprising: a) an Fc domain comprising a first Fc polypeptide and a second Fc polypeptide; b) a masking moiety (MM); and c) an IL12 polypeptide; wherein the masking moiety is fused to the first Fc polypeptide by a first linker; and optionally, wherein the masking moiety further comprises a second linker; wherein the IL12 polypeptide is fused to the second Fc polypeptide by a third linker; optionally, wherein at least one of the first, second or third linkers is protease cleavable; and wherein the IL12 activity of the masked IL12 fusion protein is attenuated as compared to the IL12 activity of a control IL12 polypeptide.

One aspect of the present disclosure provides a masked IL12 fusion protein, comprising: a) an Fc domain comprising a first Fc polypeptide and a second Fc polypeptide; b) a first MM and a second MM; and c) an IL12 polypeptide; wherein the IL12 polypeptide comprises a p35 polypeptide and a p40 polypeptide; wherein the first MM is fused to the first Fc polypeptide by a first linker; wherein the p35 polypeptide is fused to the first MM by a second linker; wherein the second MM is fused to the second Fc polypeptide by a third linker; and wherein the p40 polypeptide is non-covalently bound to the p35 polypeptide; and wherein at least one of the first, second or third linkers is protease cleavable; and wherein the IL12 activity of the masked IL12 fusion protein is attenuated as compared to the IL12 activity of the IL12 containing polypeptide released after cleavage of the at least one protease cleavable linker.

One aspect of the present disclosure provides a masked IL12 fusion protein, comprising: a) an Fc domain comprising a first Fc polypeptide and a second Fc polypeptide; b) a first MM and a second MM; and c) an IL12 polypeptide; wherein the IL12 polypeptide comprises a p35 polypeptide and a p40 polypeptide; wherein the p35 polypeptide is fused to the first Fc polypeptide by a first linker; wherein the first MM is fused to the p35 polypeptide by a second linker; wherein the second MM is fused to the second Fc polypeptide by a third linker; and wherein the p40 polypeptide is non-covalently bound to the p35 polypeptide; and wherein at least one of the first, second or third linkers is protease cleavable; and wherein the IL12 activity of the masked IL12 fusion protein is attenuated as compared to the IL12 activity of the IL12 containing polypeptide released after cleavage of the at least one protease cleavable linker. In some embodiments, the first MM is fused to the C-terminal end of the first Fc polypeptide and wherein the second MM is fused to the C-terminal end of the second Fc polypeptide. In some embodiments, the p35 polypeptide is fused to the N-terminal end of the first Fc polypeptide and wherein the second MM is fused to the N-terminal end of the second Fc polypeptide.

One aspect of the present disclosure provides a composition comprising any of the masked IL12 fusion proteins described herein and a pharmaceutically acceptable excipient.

One aspect of the present disclosure provides a method of treating cancer in a subject comprising administering to the subject a therapeutically effective amount of a composition comprising any of the masked IL12 fusion proteins described herein and a pharmaceutically acceptable excipient.

One aspect of the present disclosure provides an isolated nucleic acid encoding a masked IL12 fusion protein as described herein.

One aspect of the present disclosure provides an expression vector comprising an isolated nucleic acid encoding a masked IL12 fusion protein as described herein.

One aspect of the present disclosure provides an isolated host cell comprising an isolated nucleic acid encoding a masked IL12 fusion protein as described herein or an expression vector comprising such an isolated nucleic acid.

One aspect of the present disclosure provides a method of making a masked IL12 fusion protein comprising culturing a host cell comprising an isolated nucleic acid encoding a masked IL12 fusion protein as described herein or an expression vector comprising such an isolated nucleic acid, under conditions suitable for expression of the masked IL12 fusion protein and optionally, recovering the masked IL12 fusion protein from the host cell culture medium.

One aspect of the present disclosure provides a masked interleukin 23 (IL23) fusion protein, comprising: a) an Fc domain comprising a first Fc polypeptide and a second Fc polypeptide; b) a masking moiety; c) a first protease cleavable linker; and d) an IL23 polypeptide; wherein the masking moiety is fused to the first Fc polypeptide by the first protease cleavable linker; and optionally, wherein the masking moiety further comprises a second protease cleavable linker; wherein the IL23 polypeptide is fused to the second Fc polypeptide; and wherein the IL23 activity of the masked IL23 fusion protein is attenuated as compared to the IL23 activity of the IL23 containing polypeptide released after cleavage of the protease cleavable linker. In some embodiments, the IL23 is a single chain IL23 polypeptide selected from a single chain IL23 polypeptide having the orientation p19-linker-p40 or p40-linker-p19. In some embodiments, the single chain IL23 polypeptide is a p40-linker-p19 polypeptide that is fused to the second Fc polypeptide at the p40 polypeptide. In some embodiments, the single chain IL23 polypeptide is a p19-linker-p40 polypeptide that is fused to the second Fc polypeptide at the p19 polypeptide. In some embodiments, the single chain IL23 polypeptide is fused to the c-terminal end of the second Fc polypeptide. In some embodiments, the single chain IL23 polypeptide is fused to the c-terminal end of the second Fc polypeptide and the masking moiety is fused to the c-terminal end of the first Fc polypeptide.

One aspect of the present disclosure provides a recombinant polypeptide comprising a protease cleavable linker (PCL) wherein the protease cleavable linker comprises the amino acid sequence MSGRSANA (SEQ ID NO:10). In some embodiments, the recombinant polypeptide comprises two heterologous polypeptides, a first polypeptide located amino (N) terminally to the PCL and a second polypeptide located carboxyl (C) terminally to the PCL. In some embodiments, the two heterologous polypeptides are selected from a cytokine polypeptide, an antibody, an antigen-binding fragment of an antibody and an Fc domain. In some embodiments, the recombinant polypeptide comprises a cytokine polypeptide, a MM, and an Fc domain. In some embodiments, the MM is a single-chain Fv (scFv) antibody fragment that binds to the cytokine or a cytokine receptor polypeptide or a cytokine-binding fragment thereof. In some embodiments, the recombinant polypeptide comprises an antibody or antigen binding fragment thereof that binds a target, and a MM that binds to the antibody or antigen binding fragment thereof and blocks binding of the antibody or antigen binding fragment thereof to the target.

One aspect of the present disclosure provides an isolated polypeptide comprising a PCL, wherein the PCL comprises the amino acid sequence of SEQ ID NO:10, wherein the PCL is a substrate for a protease, wherein the isolated polypeptide comprises at least one moiety (M) selected from the group consisting of a moiety that is located amino (N) terminally to the PCL (MN), a moiety that is located carboxyl (C) terminally to the PCL (MC), and combinations thereof, and wherein the MN or MC is selected from the group consisting of an antibody or antigen binding fragment thereof; a cytokine or a functional fragment thereof; a MM; a cytokine receptor or a functional fragment thereof; an immunomodulatory receptor, or functional fragment thereof; an immune checkpoint protein or a functional fragment thereof; a tumor associated antigen; a targeting domain; a therapeutic agent; an antineoplastic agent; a toxic agent; a drug; and a detectable label.

### BRIEF DESCRIPTION OF THE DRAWINGS

FIG. 1: Schematic diagrams of parental non-masked IL12 HetFc fusion protein variants

FIGS. 2A-2B: Three-dimensional structure of uPa (FIG. 2A, 5HGG.pdb) and matriptase (FIG. 2B, 3BN9.pdb) with a polypeptide bound to the catalytic site demonstrating the potential interactions of the 8 residue centered around the cleavage site between P1 and P1′.

FIG. 3A and FIG. 3B: Schematic diagrams of the one-armed antibody format and variant(s) used to develop protease specific cleavable sites, where P4-P4′ or X indicates the localization of the cleavage site.

FIGS. 4A-4B: Kinetic curves reporting cleavage of one-armed mesothelin blocked variants by matriptase (FIG. 4A) or uPa (FIG. 4B) over time.

FIG. 5: Schematic diagrams of masked IL12 HetFc fusion protein variants derived from parental non-masked variant v22951

FIG. 6: Schematic diagrams of masked IL12 HetFc fusion protein variants derived from parental non-masked variant v22945

FIG. 7: Schematic diagrams of masked IL12 HetFc fusion protein variants derived from parental non-masked variant v22946

FIG. 8: Schematic diagrams of masked IL12 HetFc fusion protein variants derived from parental non-masked variant v22948

FIG. 9: Schematic diagrams of masked IL12 HetFc fusion protein variants derived from parental non-masked variant v23086

FIGS. 10A, 10B, and 10C show effects of lead untreated or matriptase treated (+M) parental and antibody masked IL12 HetFc fusion v31277 on relative NK cell abundance.

FIG. 11A-FIG. 11D show effects of untreated or matriptase treated (+M) cleavable and noncleavable parental and antibody masked IL12 HetFc fusion protein variants on relative NK cell abundance.

FIG. 12A-FIG. 12O show effects of untreated or matriptase treated (+M) parental and antibody masked IL12 HetFc fusion protein variants on relative NK cell abundance.

FIG. 13A-FIG. 13C show effects of best untreated or matriptase treated (+M) parental and receptor masked IL12 HetFc fusion v32045 on relative NK cell abundance.

FIGS. 14A and 14B show effects of untreated or matriptase treated (+M) cleavable and noncleavable parental and receptor masked IL12 HetFc fusion protein variants on relative NK cell abundance.

FIG. 15A-FIG. 15E show effects of untreated or matriptase treated (+M) parental and receptor masked IL12 HetFc fusion protein variants on relative NK cell abundance.

FIG. 16A and FIG. 16B show effects of heparin binding mutant IL12 HetFc fusion proteins on relative NK cell abundance.

FIG. 17A-FIG. 17E show effects of untreated or matriptase treated (+M) heparin binding mutant parental and masked IL12 HetFc fusion protein variants on relative NK cell abundance.

FIG. 18A-FIG. 18F show effects of untreated or matriptase treated (+M) parental, antibody and receptor masked IL12 HetFc fusion protein variants derived from parental variant 22951 on CD8+ T cell IFNγ release.

FIG. 19A-FIG. 19D show effects of parental, non-masked IL12 HetFc fusion protein variants on the survival of mice engrafted with human PBMCs.

FIG. 20: Serum exposure of parental, non-masked IL12 HetFc fusions in mice engrafted with human PBMCs.

FIG. 21: Schematic diagrams of double-masked IL12 HetFc fusion protein variants.

FIG. 22: shows a schematic drawing of the structure of certain fusion proteins described in Example 16. By fusing PD-1 (checkered) and PD-L1 (striped) to the N termini of heavy and light chain, respectively, the paratope of a Fab (grey) can be sterically blocked by the Ig superfamily heterodimer that is formed between the two. Upon removal of one side of this mask via the TME-specific, proteolytic cleavage (bolt) of one of the linkers that is introduced between the masking domain and the Fab, part of the mask can be released and binding to the target can be restored. Furthermore, the part of the mask that remains covalently attached to the Fab adds functionality by binding to its immunomodulatory partner.

FIG. 23: shows a schematic drawing of a modified bispecific CD3×Her2 Fab×scFv Fc fusion protein described in Example 16. One arm of the antibody-like molecule contains the anti CD3 Fab that is blocked by a PD-1/PD-L1 mask, while the other arm contains an anti-Her2 scFv.

FIG. 24: shows reducing Caliper profiles of representative variants before (-uPa) and after uPa treatment (+uPa). Profiles for unmasked (30421), masked but uncleavable (30423), and masked cleavable variants (30430, 30436, 31934) are shown.

FIG. 25: shows native binding results of CD3 targeted variants to Jurkat cells as determined by ELISA. Results are shown for an unmasked variant (30421), constructs with only the PD-L1 or PD-1 moiety attached (31929, 31931), and variants with a full, uncleavable mask (30423) or with a full mask and a cleavable PD-L1 or PD-1 moiety (30430, 30436). For samples of variants 30423, 30430, 30436, uPa untreated (-uPa) and treated (+uPa) samples were tested.

FIG. 26: shows cell killing of JIMT-1 tumor cells by Pan T-cells as determined in a TDCC assay after treatment with engineered variants cross-linking T-cells and tumor cells. Results are portrayed for an unmasked variant (30421), a construct with only the PD-1 moiety attached to the heavy chain (31929), and variants with a full, uncleavable mask (30423) or with a full mask and a cleavable PD-L1 moiety on the light chain (30430). For variant 30430 uPa untreated (-uPa) and treated (+uPa) samples were tested. An irrelevant anti-RSV antibody (22277) was used as a negative control.

FIG. 27A and FIG. 27B: shows reduced potency in a CD8+ T cell IFNγ release assay induced by untreated double antibody masked IL12HetFc fusion protein compared to parental variant 30806. Matriptase treatment (+M) of double masked variant restores activity similar to 30806.

FIGS. 28A, 28B and 28C: show a range of reduced potency in a CD8+ T cell IFNγ release assay induced by non-masked and antibody masked IL12 HetFc fusion protein variants with mutations in IL-12p35 and p40 compared to parental variant 30806.

FIG. 29: shows that altering cleavable linker lengths in untreated antibody masked IL12 HetFc fusion protein variants has minimal effect on potency in a CD8+ T cell IFNγ release assay.

FIG. 30: shows solid human tumors from indications that may respond to treatment with protease cleavable IL-12Fc fusions due to the presence of high immune cell infiltration (CIBERSORT score) and high levels of proteases (transcripts per million).

FIG. 31: shows masked and non-masked IL12 HetFc fusions display antibody-like pharmacokinetic properties in stem cell humanized mice.

FIG. 32: Schematic diagrams of masked and non-masked IL12 HetFc fusion protein variants, where p35 and p40 domains may or may not contain additional mutations to reduce IL12 potency.

FIG. 33: shows the structures and sequence compositions of variants tested in Example 16, corresponding with Table 16.

### DETAILED DESCRIPTION

The present disclosure relates to masked cytokine fusion proteins that are unmasked or activated by protease cleavage. In particular, the present disclosure relates to masked IL12 family member cytokines and more specifically, to masked IL12 and IL23 fusion proteins. The present disclosure further provides compositions and kits comprising the masked cytokines described herein and methods of using the compositions for the treatment of a variety of diseases.

IL12 is an immunostimulatory cytokine capable of driving anti-tumor responses by the innate and adaptive immune cells. The use of IL12 as a therapeutic has been extensively studied in pre-clinical models of cancer including mouse models of melanoma, renal cell carcinoma, mammary carcinoma, and colon carcinoma. The anti-tumor activity of IL12 administrations has been shown even when IL12 was administered at later stages with large, established tumors in mice. The potent anti-tumor effects of IL12 in preclinical models led to clinical trials of recombinant IL12. Unfortunately, toxicities including treatment related deaths of two patients resulted in halting of clinical trials for recombinant IL12. It is also noteworthy that recombinant cytokines have poor PK due to their small size. The present disclosure provides IL12 fusion proteins that circumvent the toxicities by blocking the cytokine activity with the use of a masking moiety that blocks IL12 binding and/or activity. The IL12 fusion protein masking moiety is designed to be released upon reaching the tumor microenvironment or other targeted anatomical location. Upon release of the masking moiety in the tumor microenvironment or other targeted anatomical location, the IL12 fusion protein recovers anti-tumor activity. The toxicities associated with IL12 administrations are reduced by locally limiting the activity of the cytokines, e.g., limiting the cytokine activity to the tumor microenvironment or other particular location in the body (such as liver, kidney, lymph node etc.). The present disclosure also provides for improved pharmacokinetics of IL12 by fusion to an Fc domain.

### Definitions

Unless defined otherwise, all technical and scientific terms used herein have the same meaning as commonly understood by one of ordinary skill in the art.

As used herein, the term “about” refers to an approximately ±10% variation from a given value, unless otherwise indicated. It is to be understood that such a variation is always included in any given value provided herein, whether or not it is specifically referred to.

The use of the word “a” or “an” when used herein in conjunction with the term “comprising” may mean “one,” but it is also consistent in certain embodiments with the meaning of “one or more,” “at least one” or “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, use or method, denotes that additional elements and/or method steps may be present, but that these additions do not materially affect the manner in which the recited composition, method or use functions. The term “consisting of” when used herein in connection with a composition, use or method, excludes the presence of additional elements and/or method steps. A composition, use or method described herein as comprising certain elements and/or steps may also, in certain embodiments consist essentially of those elements and/or steps, and in other embodiments consist of those elements and/or steps, whether or not these embodiments are specifically referred to.

By “fused” is meant that the components (e.g. a cytokine molecule and an Fc domain polypeptide or a masking moiety and an Fc domain polypeptide) are linked by peptide bonds, either directly or via one or more peptide linkers.

As used herein, the term “single-chain” refers to a molecule comprising amino acid monomers linearly linked by peptide bonds. In certain embodiments, one of the cytokine protein or domains is a single-chain cytokine molecule, i.e. an IL12 molecule wherein the p35 and the p40 domains are connected by a peptide linker to form a single peptide chain; or an IL23 molecule wherein the p19 and the p40 domains are connected by a peptide linker to form a single peptide chain.

It is contemplated that any embodiment discussed herein can be implemented with respect to any method, use or composition disclosed herein.

Particular features, structures and/or characteristics described in connection with an embodiment disclosed herein may be combined with features, structures and/or characteristics described in connection with another embodiment disclosed herein in any suitable manner to provide one or more further embodiments.

It is also to be understood that the positive recitation of a feature in one embodiment, serves as a basis for excluding the feature in an alternative embodiment. For example, where a list of options is presented for a given embodiment or claim, it is to be understood that one or more option may be deleted from the list and the shortened list may form an alternative embodiment, whether or not such an alternative embodiment is specifically referred to.

### Masked IL12/Protease Activatable IL12 Fusion Proteins

The present disclosure provides masked cytokine fusion proteins and, in particular, provides masked IL12 and IL23 fusion proteins, also referred to herein as masked IL12 HetFc fusion proteins. The masked IL12 fusion proteins described herein comprise an IL12 polypeptide, an Fc domain comprising a first Fc polypeptide and a second Fc polypeptide; a masking moiety (MM) that reduces, inhibits or blocks IL12 activity; and in certain embodiments, at least one protease cleavable linker; and optionally, additional linkers which may or may not also be protease cleavable. In certain embodiments, the masked IL12 fusion proteins may comprise two or more MM. Generally, the function of the masked IL12 fusion protein is to provide a biologically active IL12 protein that has reduced toxicity. In certain embodiments, the masked IL12 fusion proteins herein have therapeutically effective activity at local target sites, such as the tumor microenvironment (TME), while having substantially attenuated activity in the periphery. The masked IL12 fusion proteins herein provide an active IL12 protein with a broader therapeutic window. As used herein, “therapeutic window” refers to the range of dosages which can treat disease effectively without having toxic effects; e.g., as is in the area between adverse response and desired response is the therapeutic window. Examples of toxic effects of IL12 administration include, without limitation: skin toxicity, local inflammation, stomatitis, systemic inflammation, fatigue, weight loss, emesis, anorexia, hematologic toxicities, such as anemia, lymphopenia, neutropenia, thrombocytopenia, hypoproteinemia, hypophosphatemia, and hypocalcemia, enlargement of lymph nodes, splenomegaly, and bone marrow hyperplasia, bone marrow toxicities, muscle toxicities, neurologic toxicities, hepatic toxicities such as hepatic dysfunction, elevated transaminases, elevated aspartate aminotransferase (AST), elevated alanine aminotransferase (ALT), elevated alkaline phosphatase, hyperbilirubinemia, and hypoalbuminemia, elevated creatinine, diarrhea, dyspnea, and gastrointestinal hemorrhage. In some embodiments, toxic effects refer to dose-limiting toxicities. Other toxic effects of IL12 administration are known to those of ordinary skill in the art.

### Masked IL12 Fusion Protein Configurations

“Masked IL12 fusion protein” as used herein is specifically meant to include fusion proteins described herein comprising any cytokine from the IL12 family of heterodimeric cytokines and therefore, is meant specifically to include IL12 and IL23 masked fusion proteins. In certain places, “masked cytokine fusion protein” may be used and is similarly meant to include masked IL12 or IL23 fusion proteins. Additionally, the masked IL12 fusion proteins may be referred to herein as “masked HetFc IL12 fusion proteins” as the fusion proteins are in some embodiments made with the modified Fc polypeptides described herein. The terminology “masked IL12 fusion protein” and “masked cytokine fusion protein” also are meant to include any masked HetFc IL12 fusion proteins.

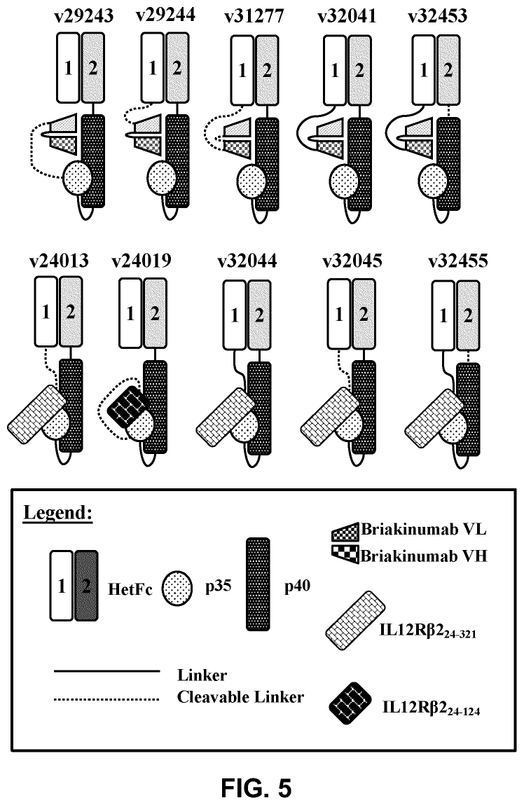

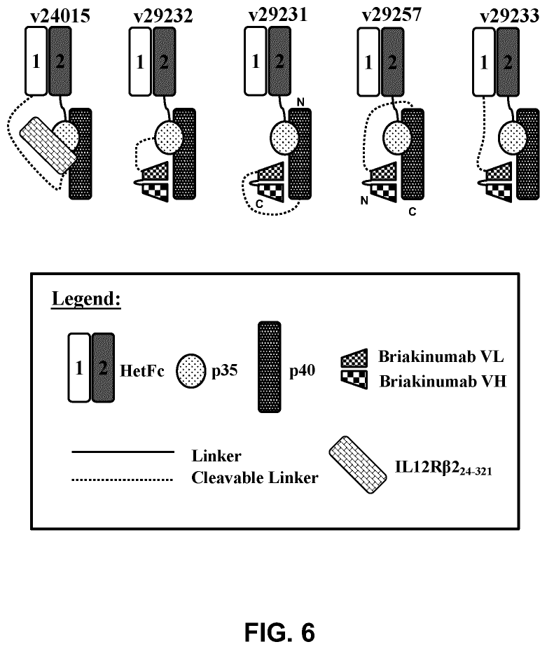

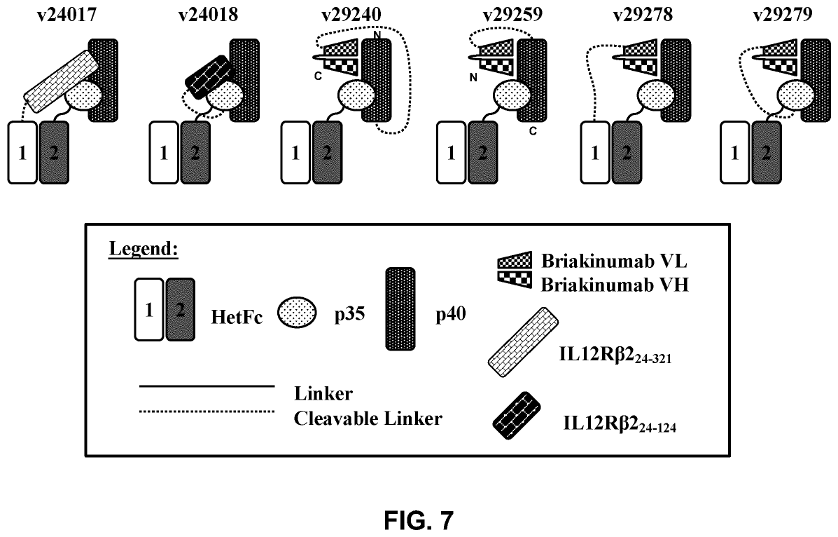

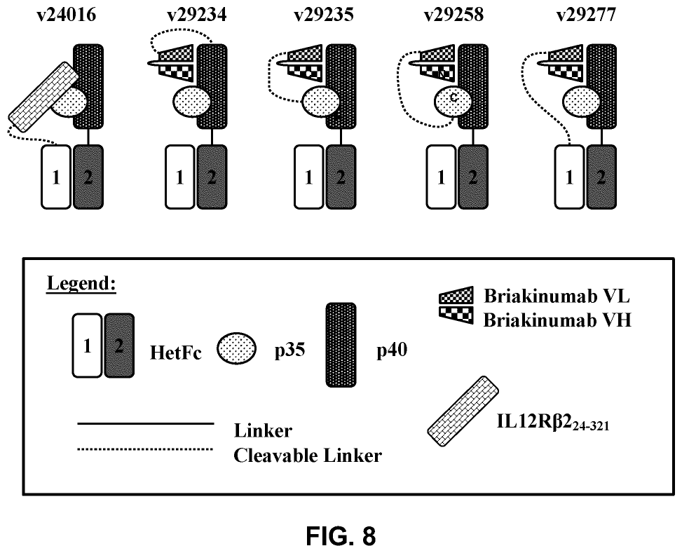

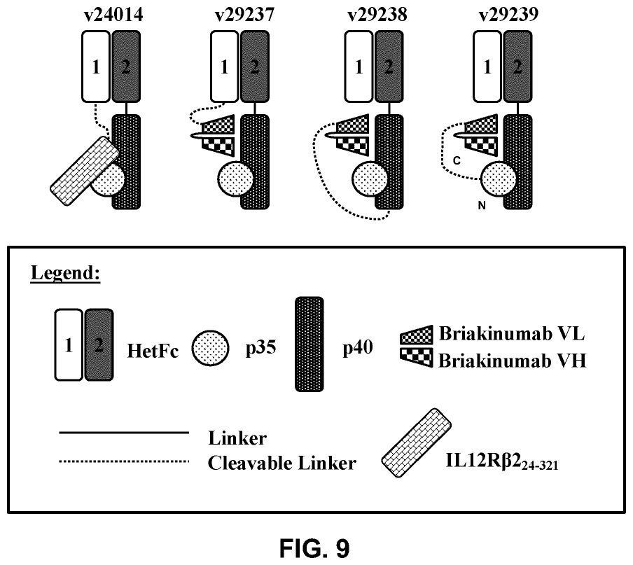

The masked IL12 fusion proteins of the present disclosure are provided in a variety of structural configurations (domain structures) that have been shown to provide unexpected benefits as compared to other configurations, in particular, improved masking, improved manufacturability, improved cleavage of the protease cleavable linker and/or improved IL12 activity post-cleavage. Exemplary structural configurations of the masked IL12 fusion proteins of the present disclosure are provided in FIGS. 5-9, 21 and 32 and are outlined in Table A below. Certain exemplary masked IL12 fusion proteins and unmasked parental IL12 fusion proteins described herein are provided in the Examples and are shown in Tables 1, 2, 10, 11, 14, 15, 16, and in Table 24 with specific reference to SEQ ID NOs in Table 25.

TABLE A
Masked Cytokine Configurations

| | | Chain 1 (HetFc 1) | Chain 2 (HetFc 2) | Chain 3 (other) |
|---|---|---|---|---|
| FIG. | Variant | (N to C) | (N to C) | (N to C) |
| 1 | v22945 | HetFc-p35 | HetFc | p40 |
| 1 | v22946 | p35-L-HetFc | HetFc | p40 |
| 1 | v22948 | p40-L-HetFc | HetFc | p35 |
| 1 | v22949 | HetFc-p35 | HetFc-p35 | p40 |
| 1 | v22951 | HetFc-L1^{a}-p40-L2- | HetFc | NA |
| | | p35 | | |
| 1 | v23086 | HetFc-L-p40 | HetFc | p35 |
| 1 | v23087 | HetFc-L1-p40 | HetFc-L2-p40 | p35 |
| | v23046 | HetFc-p19 | HetFc | p40 |
| | v23048 | p19-L-HetFc | HetFc | p40 |
| | v23051 | p40-L-HetFc | HetFc | p19 |
| | v23088 | HetFc-L-p40 | HetFc | p19 |
| | v23091 | HetFc-L1-p40-L2-p19 | HetFc | NA |
| 5 | v24013 | HetFc-PCL1- | HetFc-L2-p40-L3- | NA |
| | | IL12Rβ2₂₄₋₃₂₁ | p35 | |
| 5 | v24019 | HetFc | HetFc-L1-p40-L2- | NA |
| | | | p35-PCL3- | |
| | | | IL12Rβ2₂₄₋₁₂₄ | |
| 5 | v29243 | HetFc-L1-p40-L2- | HetFc | NA |
| | | p35-PCL3-Briak_{VL}- | | |
| | | L4-Briak_{VH} | | |
| 5 | v29244 | HetFc-PCL1-Briak_{VL}- | HetFc-L3-p40-L4- | NA |
| | | L2-Briak_{VH} | p35 | |
| 5 | v31277 | HetFc-PCL1-Briak_{VH}- | HetFc-L3-p40-L4- | NA |
| | | PCL2-Briak_{VL} | p35_{ΔR} | |
| 5 | v32041 | HetFc-L1-Briak_{VH}-L2- | HetFc-L3-p40-L4- | NA |
| | | Briak_{VL} | p35_{ΔR} | |
| 5 | v32044 | HetFc-L1-IL12Rβ2₂₄₋₃₂₁ | HetFc-L2-p40-L3- | NA |
| | | | p35_{ΔR} | |
| 5 | v32045 | HetFc-PCL1- | HetFc-L2-p40-L3- | NA |
| | | IL12Rβ2₂₄₋₃₂₁ | p35_{ΔR} | |
| 5 | v32453 | HetFc-L1-Briak_{VH}-L2- | HetFc-PCL3-p40-L4- | NA |
| | | Briak_{VL} | p35_{ΔR} | |
| 5 | v32455 | HetFc-L1-IL12Rβ2₂₄₋₃₂₁ | HetFc-PCL2-p40-L3- | NA |
| | | | p35_{ΔR} | |
| 6 | v24015 | HetFc-PCL- | HetFc-p35 | p40 |
| | | IL12Rβ2₂₄₋₃₂₁ | | |
| 6 | v29232 | HetFc-p35-PCL1- | HetFc | p40 |
| | | Briak_{VL}-L2-Briak_{VH} | | |
| 6 | v29257 | HetFc-p35 | HetFc | Briak_{VH}-L1-Briak_{VL}- |
| | | | | PCL2-p40 |
| 6 | v29231 | HetFc-p35 | HetFc | p40-PCL1-Briak_{VL}- |
| | | | | L2-Briak_{VH} |
| 6 | v29233 | HetFc-PCL1-Briak_{VL}- | HetFc-p35 | p40 |
| | | L2-Briak_{VH} | | |
| 7 | v24017 | IL12Rβ2₂₄₋₃₂₁-PCL1- | p35-L2-HetFc | p40 |
| | | HetFc | | |
| 7 | v24018 | IL12Rβ2₂₄₋₁₂₄-PCL1- | HetFc | p40 |
| | | p35-L2-HetFc | | |
| 7 | v29240 | p35-L1-HetFc | HetFc | p40-PCL2-Briak_{VL}- |
| | | | | L3-Briak_{VH} |
| 7 | v29259 | p35-L1-HetFc | HetFc | Briak_{VH}-L2-Briak_{VL}- |
| | | | | PCL3-p40 |
| 7 | v29278 | Briak_{VH}-L1-Briak_{VL}- | p35-L3-HetFc | p40 |
| | | PCL2-HetFc | | |
| 7 | v29279 | Briak_{VH}-L1-Briak_{VL}- | HetFc | p40 |
| | | PCL2-p35-L3-HetFc | | |
| 8 | v24016 | IL12Rβ2₂₄₋₃₂₁-PCL1- | p40-L2-HetFc | p35 |
| | | HetFc | | |
| 8 | v29234 | Briak_{VH}-L1-Briak_{VL}- | HetFc | p35 |
| | | PCL2-p40-L3-HetFc | | |
| 8 | v29235 | p40-L1-HetFc | HetFc | p35-PCL2-Briak_{VL}- |
| | | | | L3-Briak_{VH} |
| 8 | v29258 | p40-L1-HetFc | HetFc | Briak_{VH}-L2-Briak_{VL}- |
| | | | | PCL3-p35 |
| 8 | v29277 | Briak_{VH}-L1-Briak_{VL}- | p40-L3-HetFc | p35 |
| | | PCL2-HetFc | | |
| 9 | v24014 | HetFc-PCL1- | HetFc-L2-p40 | p35 |
| | | IL12Rβ2₂₄₋₃₂₁ | | |
| 9 | v29237 | HetFc-PCL1-Briak_{VL}- | HetFc-L3-p40 | p35 |
| | | L2-Briak_{VH} | | |
| 9 | v29238 | HetFc-L1-p40-PCL2- | HetFc | p35 |
| | | Briak_{VL}-L3-Briak_{VH} | | |
| 9 | v29239 | HetFc-L1-p40 | HetFc | p35-PCL2-Briak_{VL}- |
| | | | | L3-Briak_{VH} |
| 21 | v32867 | HetFc-PCL1-Briak_{VH}- | HetFc-L3-p40-L4- | NA |
| | | PCL2-Briak_{VL} | p35_{ΔR}-PCL5-h6F6_{VL}- | |
| | | | L6-h6F6_{VH} | |
| 21 | v32868 | HetFc-PCL1-Briak_{VH}- | HetFc-L5-p40-L6- | NA |
| | | PCL2-Briak_{VL}-PCL3- | p35_{ΔR} | |
| | | h6F6_{VL}-L4-h6F6_{VH} | | |
| 21 | v32869 | HetFc-PCL1-Briak_{VH}- | HetFc-L5-p40-L6- | NA |
| | | PCL2-Briak_{VL}-PCL3- | p35_{ΔR} | |
| | | h6F6_{VH}-L4-h6F6_{VL} | | |
| 21 | v32870 | HetFc-PCL1- | HetFc-L2-p40-L3- | NA |
| | | IL12Rβ1₂₄₋₂₄₀ | p35_{ΔR}-PCL4- | |
| | | | IL12Rβ2₂₄₋₃₂₁ | |
| 21 | v32871 | HetFc-PCL1- | HetFc-L3-p40-L4- | NA |
| | | IL12Rβ2₂₄₋₁₂₄-PCL2- | p35_{ΔR} | |
| | | IL12Rβ1₂₄₋₂₄₀ | | |
| 21 | v32873 | IL12Rβ2₂₄₋₁₂₄-PCL1- | IL12Rβ1₂₄₋₂₄₀-PCL3- | p40 |
| | | p35-L2-HetFc | HetFc | |
| 21 | v32895 | HetFc-PCL1- | HetFc-PCL2- | p40 |
| | | IL12Rβ1₂₄₋₂₄₀ | IL12RB2₂₄₋₁₂₄-PCL3- | |
| | | | p35 | |
| | Pv1^{b} | HetFc-PCL1-Briak_{VH}- | HetFc-PCL3-p40-L4- | NA |
| | | PCL2-Briak_{VL} | p35_{ΔR} | |
| 32 | v32862^{c} | HetFc-PCL1-Briak_{VH}- | HetFc-L3-p40-L4- | NA |
| | | L2-Briak_{VL} | p35_{ΔR} | |
| 32 | v35425 | HetFc-PCL1-Briak_{VH}- | HetFc-L3-p40-L4- | NA |
| | | L2-Briak_{VL} | P35_{ΔR}(F39S_Y40S_Y167S) | |
| 32 | v35456 | HetFc-PCL1-Briak_{VH}- | HetFc-L3-p40-L4- | NA |
| | | L2-Briak_{VL} | p35_{ΔR}-PCL5-h6F6_{VL}- | |
| | | | L6-h6F6_{VH} | |
| 32 | v36190 | HetFc-PCL1-Briak_{VH}- | HetFc-L3- | NA |
| | | L2-Briak_{VL} | p40(D41S_E45R_K58S_E59S_K195D)- | |
| | | | L4-p35_{ΔR} | |
| 32 | v35436 | HetFc-L1-Briak_{VH}-L2- | HetFc-L3-p40-L4- | NA |
| | | Briak_{VL} | p35_{ΔR} | |
| 32 | v35437 | HetFc-L1-Briak_{VH}-L2- | HetFc-L3-p40-L4- | NA |
| | | Briak_{VL} | p35_{ΔR}(F39S_Y40S_Y167S) | |
| 32 | v35457 | HetFc-L1-Briak_{VH}-L2- | HetFc-L3-p40-L4- | NA |
| | | Briak_{VL} | p35_{ΔR}-L5-h6F6_{VL}- | |
| | | | L6-h6F6_{VH} | |
| 32 | v36193 | HetFc-L1-Briak_{VH}-L2- | HetFc-L3- | NA |
| | | Briak_{VL} | p40(D41S_E45R_K58S_E59S_K195D)- | |
| | | | L4-p35_{ΔR} | |
| 32 | v33507 | HetFc-PCL1-Briak_{VH}- | HetFc-L3-p40-L4- | NA |
| | | PCL2-Briak_{VL} | P35_{ΔR}(F39S_Y40S_Y167S) | |
| 32 | v33510 | HetFc-PCL1-Briak_{VH}- | HetFc-L3- | NA |
| | | PCL2-Briak_{VL} | p40(D41S_E45R_K58S_E59S_K195D)- | |
| | | | L4-p35_{ΔR} | |

^{a}The numbering of the linkers (L, PCL) is for clarity only and the numbers are interchangeable. Any given L or PCL may have a different number depending on the configuration or geometry.
^{b}Identical to v31277 (FIG. 5) but adding the cleavable linker from v32453.
^{c}v32862 is identical to v31277 except that the linker between Briak_{VH} and Briak_{VL} is not protease cleavable.

One aspect of the present disclosure provides non-masked parental IL12 fusion proteins. Such non-masked parental IL12 fusion proteins contain the domains described above for the masked IL12 fusion proteins but lack the MM and in certain embodiments, the linker attaching the MM to the rest of the fusion protein. These non-masked parental IL12 fusion proteins have not been modified by a MM and in certain embodiments are used as comparator fusion proteins where appropriate.

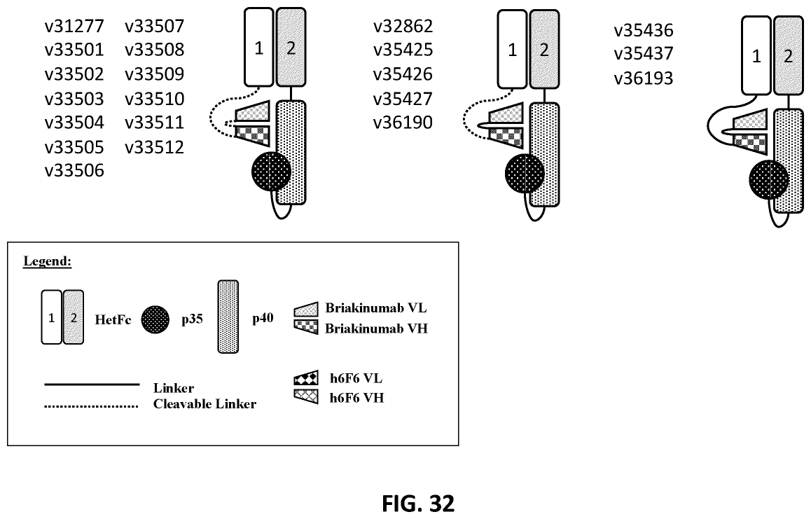

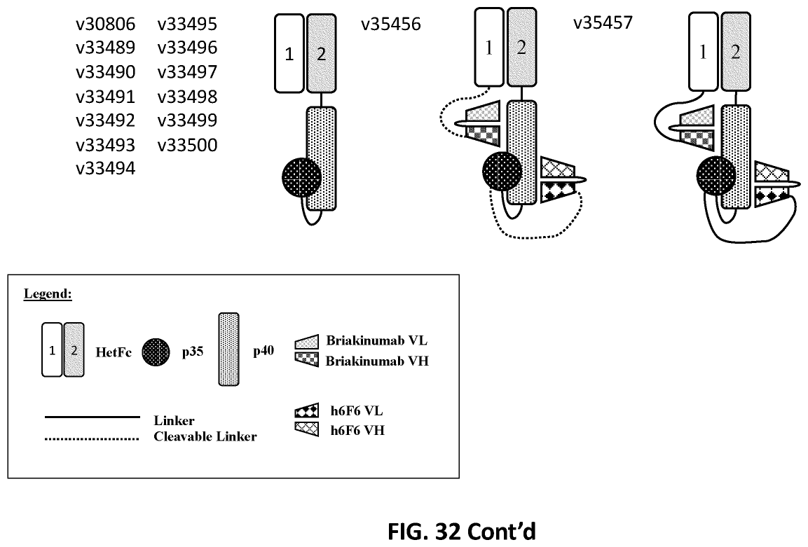

In one embodiment, the masked IL12 fusion protein has the structural configuration Fc1-L1-MM/Fc2-L2-p40-L3-p35 (see e.g., FIG. 5, variant 31277; where Fc1 is connected to Fc2 by a disulfide bond) wherein at least one of L1, L2, or L3 is a protease cleavable linker. In one embodiment, L1 is a protease cleavable linker. In a further embodiment, the MM further comprises a fourth linker. In this regard, in certain embodiments and as noted elsewhere herein, the MM may be an scFv having the structure configuration VH-L-VL or VL-L-VH and in certain embodiments, the linker between the VH and VL is optionally a protease cleavable linker (see e.g., FIG. 32, variant 32862).

It should be noted that the numbering of the linkers is for clarity only and the numbers are interchangeable. Any given linker may have a different number depending on the configuration or geometry. L1 in one geometry is not necessarily the same linker as L1 in a different geometry. In some configurations, L1 may be a protease cleavable linker and in other configurations, L1 is not a protease cleavable linker. Moreover, similar geometries may number the linkers differently.

As used herein the “IL12 containing polypeptide” or the “released IL12 polypeptide” refers to the polypeptide comprising an IL12 polypeptide that is released from the masked IL12 fusion protein after cleavage of the protease cleavable linker. This is to distinguish from a wild type IL12 or the IL12 polypeptide included in the masked fusion proteins herein (“an IL12 polypeptide” as recited in the claims). In certain embodiments, the released IL12 polypeptide is the same as the IL12 polypeptide. In other embodiments, the released IL12 polypeptide may contain amino acid sequences that correspond to portions of the protease cleavable linker and may also contain an Fc polypeptide. As a non-limiting example, in one embodiment, the masked IL12 fusion protein has the structural configuration Fc1-L1-MM/Fc2-L2-p40-L3-p35 (see e.g., v31277 or v32455 in FIG. 5; where Fc1 is connected to Fc2 by one or more disulfide bonds) wherein at least one of L1, L2, or L3 is a protease cleavable linker. In this setting, where L1 is a protease cleavable linker, the released IL12 polypeptide (released after cleavage of the protease cleavable linker) has the following structural configuration: Fc1-L1′/Fc2-L2-p40-L3-p35, where L1′ is the portion of the protease cleavable linker that remains after protease cleavage and Fc1 is connected to Fc2 by one or more disulfide bonds. As another example, using the same structural configuration shown above, where L2 is a protease cleavable linker (or L2 and L1 are both protease cleavable linkers), the released IL12 polypeptide has the following structural configuration: L2′-p40-L3-p35 where L2′ is the portion of the protease cleavable linker that remains after protease cleavage. In this example, the released IL12 polypeptide is no longer fused to an Fc. As noted elsewhere herein, the released IL12 polypeptide demonstrates recovered IL12 binding/activity as compared to the masked IL12 fusion protein.

Cleavage can be assessed by LabChip™ CE-SDS analysis. In one illustrative assay, masked IL12 HetFc fusion proteins are incubated for about 10 to about 24 hours with matriptase (R&D Systems) at a molar ratio of 1:50 (Matriptase:Protein) in buffer at a neutral pH at 37° C. Non-reducing and reducing LabChip™ CE-SDS analysis is carried out to assess the degree of digestion, and LC/MS is performed (see e.g., as described in the Examples and the Protocols described in the Examples) to identify the locations of cleavage. Recovery of IL12 activity or IL12 receptor complex binding following protease cleavage can be tested using SPR or cell based assays known in the art, such as those described herein (NK relative abundance, CD8+IFNγ release, CTLL-2 assays).

When the masked IL12 fusion protein is in the presence of the IL12 receptor complex but not sufficient enzyme or enzyme activity to cleave the protease cleavable linker, specific binding of the masked IL12 fusion protein to the IL12 receptor complex is reduced or inhibited, as compared to the IL12 polypeptide released after cleavage of the protease cleavable linker in the presence of the IL12 receptor and sufficient enzyme or enzyme activity to cleave the protease cleavable linker.

When the masked IL12 fusion protein is in the presence of the IL12 receptor complex but not sufficient enzyme or enzyme activity to cleave the protease cleavable linker, functional IL12 activity of the masked IL12 fusion protein is reduced or inhibited, as compared to the functional IL12 activity of the IL12 polypeptide released after cleavage of the protease cleavable linker in the presence of the IL12 receptor and sufficient enzyme or enzyme activity to cleave the protease cleavable linker.

By reduced or inhibited binding or activity it is meant that binding or functional IL12 activity is lower than the binding or functional IL12 activity of an appropriate control, such as wild type IL12, the released IL12 polypeptide or a corresponding unmasked parental fusion protein. The reduced or inhibited binding or activity can be expressed as reduced potency. In certain embodiments, the potency of a masked IL12 fusion protein in its masked state is reduced by about 2-fold to about 2500-fold as compared to the IL12 activity of an appropriate control, such as parental non-masked fusion proteins or the IL12 polypeptide released from the masked IL12 fusion protein after cleavage of the protease cleavable linker. The potency of a masked IL12 fusion protein as described herein is in certain embodiments reduced by about 5-fold to about 2000-fold, by about 10-fold to about 1500-fold, by about 15-fold to about 1000-fold, by about 20-fold to about 800-fold, by about 25-fold to about 600-fold, by about 25-fold to about 100-fold, by about 50-fold to about 100-fold, by about 50-fold to about 2000-fold, by about 100-fold to about 2000-fold, or by about 500-fold to about 2000-fold. In some embodiments, the potency of a masked IL12 fusion protein as described herein is reduced by about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2500, or 3000-fold. In certain embodiments, potency is reduced by more than 3500, 4000, 4500, 5000, 5500, 6000, 7000, 8000, 9000 or 10,000 fold.

When the masked IL12 fusion protein is in the presence of the IL12 receptor and sufficient enzyme or enzyme activity to cleave the protease cleavable linker (e.g., in the tumor microenvironment (TME) or other relevant in vivo location), the protease cleavable linker is cleaved and unmasks or releases a functional IL12 polypeptide, also referred to herein as the “released IL12 polypeptide”. Just as the specific binding and functional IL12 activity (potency) of the masked IL12 fusion protein is reduced or inhibited as compared to the IL12 polypeptide released after cleavage of the protease cleavable linker, the binding and functional IL12 activity of the released IL12 polypeptide released after cleavage of the protease cleavable linker is increased as compared to the masked IL12 fusion protein in its masked, uncleaved state.

Recovered IL12 activity or binding of the released IL12 polypeptide following protease cleavage can be determined as compared to wild type IL12, the uncleaved masked IL12 fusion protein (e.g., untreated with protease), parental non-masked IL12 fusion protein or other appropriate control. Thus, in certain embodiments, the released IL12 polypeptide has between 2-fold and 5000-fold activity or binding as compared to an appropriate control. The recovered IL12 activity can also be expressed as x-fold increased potency as compared to an appropriate control. In certain embodiments, the potency or activity of a released IL12 polypeptide is increased by about 10-fold to about 2500-fold as compared to the IL12 activity of an appropriate control, such as an uncleaved masked IL12 fusion protein. The potency of a released IL12 polypeptide as described herein is in certain embodiments increased by about 5-fold to about 2000-fold, by about 10-fold to about 1500-fold, by about 15-fold to about 1000-fold, by about 20-fold to about 800-fold, by about 25-fold to about 600-fold, by about 25-fold to about 100-fold, by about 50-fold to about 100-fold, by about 50-fold to about 2000-fold, by about 100-fold to about 2000-fold, or by about 500-fold to about 2000-fold as compared to an untreated uncleaved masked control fusion protein or other appropriate control. In some embodiments, the potency of a released IL12 polypeptide as described herein is about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 9000, or 10,000-fold increased as compared to an untreated uncleaved masked control fusion protein or other appropriate control.

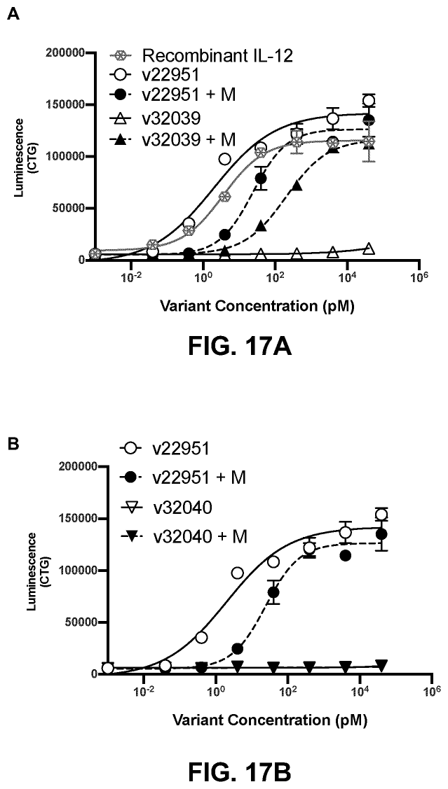

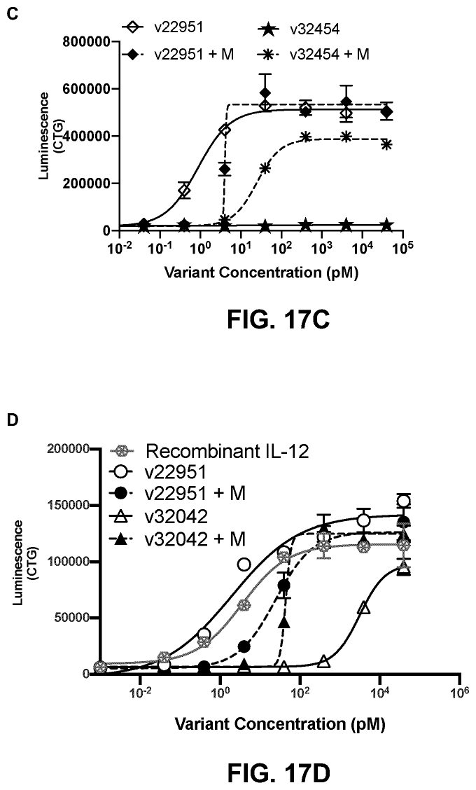

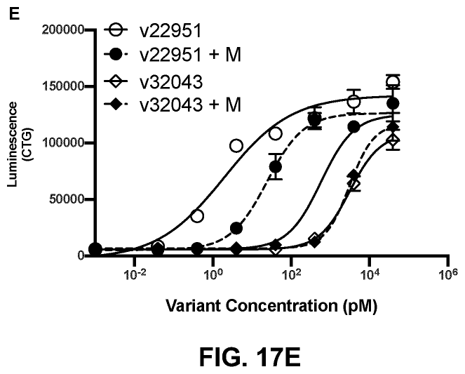

In certain embodiments, a masked IL12 fusion protein as described herein demonstrate a complete reduction in potency of the IL12 polypeptide in that IL12 activity is undetected by, e.g., an NK or other cell-based assay. In this case, the “fold reduction in potency” cannot be calculated as activity is below the limit of detection. The recovery of the IL12 activity of the released IL12 polypeptide can be expressed as within x-fold of a different comparator (see e.g., v32454, FIG. 17C).

Methods for measuring binding or functional IL12 activity are known in the art and described herein. In certain embodiments, binding activity can be measured using surface plasmon resonance (SPR). Functional IL12 activity can be measured, for example, in an NK cell relative abundance or CD8+ T cell IFNγ release assay (see e.g., Example 9).

Thus, in certain embodiments, provided herein are masked IL12 fusion proteins that exhibit, in the absence of protease, at least 5-fold, 10-fold, 15-fold, 20-fold, 30-fold, 40-fold, 50-fold, 100-fold, 200-fold, 300-fold, 400-fold, 500-fold, 600-fold, 700-fold, 800-fold, 900-fold, 1000-fold, 1200-fold, 1500-fold, 2000-fold, 2500-fold, 3000-fold, or further reduced binding activity, functional IL12 activity, or potency as compared to an appropriate control, as measured by SPR, NK cell, CD8+ T cell IFNγ release, or other appropriate assay.

### IL12 Family of Cytokines

The present disclosure provides masked IL12 fusion proteins. Interleukin 12 (IL12) was the first recognized member of a family of heterodimeric cytokines that includes IL12, IL23, IL27, IL35 and IL39. IL12 and IL23 are pro-inflammatory cytokines important for development of T helper 1 (Th-1) and T helper 17 (Th-17) T cell subsets, while IL27 and IL35 are potent inhibitory cytokines. IL39 is an important cytokine in regulating innate and/or adaptive immune response. IL12 can directly enhance the activity of effector CD4 and CD8 T cells as well as natural killer (NK) and NK T cells.

Interleukin-12 (IL12) is a heterodimeric molecule composed of an alpha chain (the p35 subunit) and a beta chain (the p40 subunit) covalently linked by a disulfide bridge to form the biologically active 70 kDa dimer. Exemplary amino acid sequences of p35 and p40 subunits of IL12 are provided in Table 24. See SEQ ID Nos: 23 and 22 and variants thereof, such as, variants of the p40 subunit comprising a modified heparin loop (amino acids 256-264 of SEQ ID NO:22). Exemplary polynucleotide sequences encoding p35 and p40 are provided in SEQ ID NOs:103 and 102, respectively, and variants thereof.

IL23 is a member of IL12 cytokine family and is also composed of two subunits: the p40 subunit that it shares with IL12 and p19. Exemplary polynucleotide and amino acid sequence of the p19 subunit of IL23 is provided in Table 24. See SEQ ID Nos: 32 and 112. The receptor for IL23 (IL23R) consists of an IL23Ra subunit in complex with an IL12R1 subunit, which is a common subunit for the IL12 receptor and interacts with Tyrosine kinase 2 (Tyk2). The IL23R is mainly expressed on immune cells, in particular T cells (e.g., Th17 and gamma delta T cells), macrophages, dendritic cells and NK cells (Duvallet et ah, 2011). It has been recently shown that non-activated neutrophils express a basal amount of IL23R and that IL23R expression is increased upon cell activation (Chen et al., 2016).

The term “a protein having the function of IL12” or “a protein having the function of IL23” encompasses mutants of a wild type IL12 or IL23 sequence, respectively, wherein the wild type sequence has been altered by one or more of addition, deletion, or substitution of amino acids. IL12 and IL23 sequences contemplated herein include IL12 and IL23 sequences from any animal, in particular any mammal, including human, mouse, dog, cat, pig, and non-human primate.

The bioactivities of IL12 are well known and include, without limitation, differentiation of naive T cells into Th1 cells, stimulation of the growth and function of T cells, production of interferon-gamma (IFN-gamma) and tumor necrosis factor-alpha (TNF-α) from T and natural killer (NK) cells, reduction of IL4 mediated suppression of IFN-gamma, enhancement of the cytotoxic activity of NK cells and CD8⁺ cytotoxic T lymphocytes, stimulation of the expression of IL12Rβ1 and IL12Rβ2, facilitation of the presentation of tumor antigens through the upregulation of MHC I and II molecules, and anti-angiogenic activity. IL12 is produced primarily by antigen-presenting cells and drives cell-mediated immunity by binding to a two-chain receptor complex that is expressed on the surface of T cells or natural killer (NK) cells. The IL12 receptor beta-1 (IL12Rβ1) chain binds to the p40 subunit of IL12. IL12p35 ligation of the second receptor chain, IL12Rβ2, confers intracellular signaling (e.g. STAT4 phosphorylation) and activation of the receptor-bearing cell (Presky et al, 1996). Studies show equal cell-based affinity of IL12 for Rβ1 and Rβ2 individually, and higher affinity for the complex (J Immunol. 1998 Mar. 1; 160(5):2174-9). IL12 also acts on dendritic cells (DC), leading to increased maturation and antigen presentation, which can allow for the initiation of a T cell response to tumor specific antigens. It also drives the secretion of IL12 by DCs, creating a positive feedback mechanism to amplify the response.

Exemplary nucleic acid and amino acid sequences for the IL12, IL23 and the masked fusion proteins described herein are provided in Tables 24.

Variants of any of the nucleic acid and amino acid sequences provided herein are also contemplated for use in the masked fusion proteins as described herein in the section entitled “Polypeptides and Polynucleotides”. In certain embodiments, the IL12 fusion protein polypeptides described herein comprise a p35 amino acid sequence as set forth in SEQ ID NO: 23. In certain embodiments, the IL12 fusion proteins described herein comprise a p40 amino acid sequence as set forth in SEQ ID NO: 22. In another embodiment, the IL12 fusion polypeptides described herein comprise a p35 amino acid sequence as set forth in SEQ ID NO: 23 and a p40 amino acid sequence as set forth in SEQ ID NO: 22. In one embodiment, the IL12 fusion proteins described herein comprise a scIL12 having the configuration p35-L-p40 or p40-L-p35. In other embodiments, the IL12 polypeptides described herein may comprise a variant of the p35 and/or p40 sequence. In this regard, the variant may comprise a variant of the nucleic acid sequence encoding the p35 or p40 amino acid sequence where the variant encodes a protein that retains IL12 functional activity as compared to the wild type IL12, or other appropriate control. A variant nucleic acid sequence may comprise at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, or higher % identity to the polynucleotide sequence encoding p35 and/or p40, such as the polynucleotide sequences set forth in SEQ ID Nos: 103 and 102. Illustrative variants of the IL12 polynucleotides include codon optimized polynucleotide sequences.

In certain embodiments, a variant may comprise a variant p35 and/or p40 polypeptide comprising at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, or higher % identity to the amino acid sequence of IL12 p35 and/or p40 as set forth in SEQ ID Nos: 23 and 22, respectively, where such variant polypeptides retain IL12 functional activity as compared to an appropriate comparator molecule comprising a wild type IL12.

In other embodiments, the IL23 polypeptides described herein may comprise a variant of the p19 and/or p40 sequence. In this regard, the variant may comprise a variant of the nucleic acid sequence encoding the p19 or p40 amino acid sequence, where the variant encodes a protein that retains IL23 functional activity as compared to the wild type IL23. A variant nucleic acid sequence may comprise at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, or higher % identity to the polynucleotide sequence encoding p19 and/or p40 as set forth in SEQ ID Nos: 112 and 102, respectively. Illustrative variants of the IL23 polynucleotides include codon optimized polynucleotide sequences.

In certain embodiments, a variant may comprise a variant p19 and/or p40 polypeptide comprising at least 50%, 55%, 60%, 65%, 70%, 75%, 80%, 85%, 86%, 87%, 88%, 89%, 90%, 91%, 92%, 93%, 94%, 95%, 96%, 97%, 98%, 99%, or higher % identity to the amino acid sequence of IL23 p19 and/or p40 as set forth in SEQ ID NOs: 32 and 22, respectively, where such variant polypeptides retain IL23 functional activity as compared to the wild type IL23.

In certain embodiments, the IL12 protein described herein has been modified to reduce heparin binding and or to be resistant to proteolytic cleavage. In this regard, the IL12 protein is modified to reduce heparin binding and/or be more resistant to proteolytic cleavage as compared to an unmodified IL12 protein. In certain embodiments, modifications are made to the IL12 protein to lower the binding affinity to heparin. In certain embodiments, modifications are made that both lower the binding affinity to heparin and result in resistance to proteolytic cleavage as compared to unmodified IL12 protein. In one embodiment, the modification to confer increased resistance to proteolytic cleavage or reduced binding to heparin is made to the p40 subunit. Illustrative modifications are described in Example 10 and 11 and are provided in Table 12. In another embodiment, the modification to confer increased resistance to proteolytic cleavage and/or reduced binding to heparin is made to the p35 subunit. In one embodiment, the N-terminal arginine of p35 is removed.

In certain embodiments assays for measuring increased resistance to proteolytic cleavage of the variants and fusion proteins described herein are known in the art and include the assays outlined in the Examples. As would be understood by one of skill in the art, assays may be modified and optimized as needed for a particular enzyme or protein to be cleaved. In one embodiment, the assay comprises incubating test proteins for a period of time with a protease at an appropriate ratio at a given pH and temperature. Non-reducing and reducing LabChip™ CE-SDS analysis is carried out to assess the degree of digestion, and LC/MS is performed to identify the locations of cleavage. In one embodiment, the assay is generally as follows: test proteins are incubated for 18 hours with protease (e.g., Matriptase (R&D Systems)) at an appropriate molar ratio, e.g., at a molar ratio of 1:50 (Matriptase:Protein) in a total reaction volume of 25 μL PBS-T pH 7.4 at 37° C. Non-reducing and reducing LabChip™ CE-SDS analysis is carried out to assess the degree of digestion, and LC/MS is performed to identify the locations of cleavage. In certain embodiments, the variants described herein demonstrate at least a 1%, 2%, 3%, 4%, 5%, 6%, 7%, 8%, 9%, 10% increase in resistance in protease cleavage (or a corresponding decrease in cleavage) as compared to wild type or comparator IL12 or IL23 polypeptides, or masked fusion proteins comprising such proteins, while retaining IL12 or IL23 functional activity. In certain embodiments, variants display up to complete resistance to protease cleavage to 24 hours contact with protease. In other embodiments, variants display up to complete resistance to protease cleavage after 1 hour-36 hours contact with protease. In another embodiment, a variant displays up to complete resistance to protease cleavage after 0.5, 1, 2, 3, 4, 5, 6, 7, 8, 9, 10, 12, 20, 24, 36, 48, or 72 hours contact with protease.

The variant cytokine polypeptides or fusion proteins comprising them as described herein, exhibit functional activity that is within 2 to 20-fold of the functional activity (e.g., IL12 or IL23) of an appropriate control, e.g., a relevant comparator fusion protein comprising a wild type cytokine (e.g., IL12 or IL23). In certain embodiments, cytokine variant polypeptides demonstrate equivalent potency as compared to wild type controls, e.g., as measured by relative abundance of NK cells, IFNγ release by CD8+ T cells, or cell signaling following receptor engagement. In other embodiments, cytokine variant polypeptides demonstrate a maximum attenuation of potency of between about 2-fold and about 20-fold. In certain embodiments, cytokine variant polypeptides or fusion proteins comprising them demonstrate attenuation of potency of between about 2-fold, 3, 4, 5, 6, 7, 8, 9, 10, 11, 12, 13, 14, 15, 16, 17, 18, 19 or about 20-fold. As noted elsewhere, IL12 is highly toxic. Accordingly, it may be desirable in certain embodiments to use a variant IL12 polypeptide having reduced potency. In certain embodiments, a variant may exhibit increased functional activity or increased potency as compared to the control, e.g., between about 2-fold and about 100-fold, or about 2-fold, 3-fold, 4-fold, 5-fold, 10-fold, 15-fold, 20-fold, 30-fold, 40-fold, 50-fold, 60-fold, 70-fold, 80-fold, 90-old, or 100-fold increased activity or potency as compared to an appropriate control. Cytokine functional activity can be measured using assays known in the art and described herein such as an NK or CTLL-2 assay or IFNγ release by CD8+ T cells.

Methods of measuring the functional activity of IL12 family cytokines are known in the art. Such methods include assays known in the art, such as assays to determine cell responsiveness to IL12 or IL23, measuring cytokine production in response to incubating appropriate cells with IL12 or IL23, measuring receptor binding and signaling activation.

In certain embodiments, IL12 activity is determined by measuring cell proliferation of cells or cell lines that are sensitive to IL12. Illustrative cells that can be used to test IL12 activity include CTLL-2 or NK cells. Such proliferation assays include assays as described, for example, by Khatri A, et al. 2007. J Immunol Methods 326(1-2):41-53; Puskas J, et al. 2011. Immunology 133(2):206-220; Hodge DL., et al. J Immunol. 2002 Jun. 15; 168(12):6090-8. Assays known in the art can be modified as desired to fit the particular cytokine being tested, such as IL12 or IL23.

In brief, a CTLL-2 assay for measuring IL12 functional activity may comprise serially diluting the recombinant proteins to be tested (e.g., a masked fusion protein as described herein) 1:5 in 50 μL of medium, then 4×10⁴ CTLL-2 cells in 100 μL of medium are added per well to a 96-well plate and incubated at 37° C. in 5% CO₂ for 18-22 h. At the end of this period, 75 μg/well of Thiazolyl Blue Tetrazolium Bromide (MTT; Sigma-Aldrich) is added and the plate is incubated for 8 h at 37° C. in 5% CO₂. Cells are lysed with 100 μL/well of 10% SDS (Gibco) acidified with HCl, incubated at 37° C. in 5% CO₂ overnight, and absorbance is read at 570 nm. Such an assay can be run on masked fusion proteins that have and have not been incubated with an appropriate protease. Thus, such assays can be used to test the masked fusion proteins described herein in the presence and absence of an appropriate protease which cleaves the protease cleavable linker and releases the mask thereby unblocking or unmasking the IL12.

In brief, an NK assay for measuring IL12 function activity can be carried out as follows: NK cells are cultured in growth medium without IL2 (assay media) for 12 hours, harvested and spun down to pellet cells. Cells are resuspended in assay media to 400 million cells/mL and 10,000 cells or 25 uL per well are added to assay plates. Variant test samples are titrated in triplicate at 1:5 dilution in 25 ul directly in 384-well black flat bottom assay plates. Recombinant cytokine (e.g., human IL12 (Peprotech, Rocky Hill, N.J.)) is included as a positive control. Plates are incubated for 3 days at 37° C. and 5% carbon dioxide. Post incubation, 25 uL/well of supernatant is transferred to non-binding 384-well plates (Greiner-Bio-One, Kremsmünster, Austria) and stored at −80° C. After supernatant removal, CellTiter-Glo® Luminescent Cell Viability reagent (Promega, Madison, Wis.) or equivalent reagent is added to plates at 25 uL/well and plates are incubated at room temperature away from light for 30 minutes. Following incubation, plate luminescence is scanned, such as on a BioTek synergy H1 plate reader (BioTek, Winooski, Vt.).

In one embodiment, IL12 activity can be determined by measuring cell signaling cascades triggered by IL12 interaction with its receptor (e.g., IL12Rβ2 and IL12Rβ1 interaction with IL12 p35-p40 heterodimers). In one embodiment, IL12 activity is determined by measuring STAT4 signaling activity using assays known in the art and commercially available for example, from Abeomics, San Diego, Calif. USA.

### Masking Moieties

The masked IL12 or IL23 fusion proteins described herein comprise a masking moiety (MM) that blocks or reduces the binding of IL12 or IL23 to its native receptor(s) and/or blocks or reduces its functional activity. In certain embodiments, the MM specifically binds to the IL12. “Specifically binds”, “specific binding” or “selective binding” means that the binding is selective for the desired antigen (in the case of the present disclosure, the MM specifically binds IL12 or IL23) and can be discriminated from unwanted or non-specific interactions. The ability of a MM to bind to and block or reduce IL12/IL23 activity can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of a MM to an unrelated protein is less than about 10% of the binding of the MM to IL12/IL23 as measured, e.g., by SPR. In certain embodiments, MM that binds to IL12/IL23 or a biologically active fragment thereof, has a dissociation constant (K_{d}) of <1 μM, <100 nM, <10 nM, <1 nM, <0.1 nM, <0.01 nM, or <0.001 nM (e.g. 10⁻⁸ M or less, e.g. from 10⁻⁸M to 10⁻¹³ M, e.g., from 10⁻⁹M to 10⁻¹³ M).

The MM of the present disclosure generally refers to an amino acid sequence present in the masked cytokine fusion protein and positioned such that it reduces the ability of the cytokine, within the context of the masked cytokine fusion protein, to specifically bind its target and/or to function. In some cases, the MM is coupled to the masked cytokine fusion protein by way of a linker and in certain embodiments, the linker is a protease cleavable linker.

In certain embodiments, the masked cytokine fusion protein comprises only non-cleavable linkers. In this regard, the MM results in the masked cytokine fusion molecule having reduced effective affinity for its target receptor, thereby reducing its toxicity. In other embodiments, as described further herein, the masked cytokine fusion protein comprises at least one protease cleavable linker.

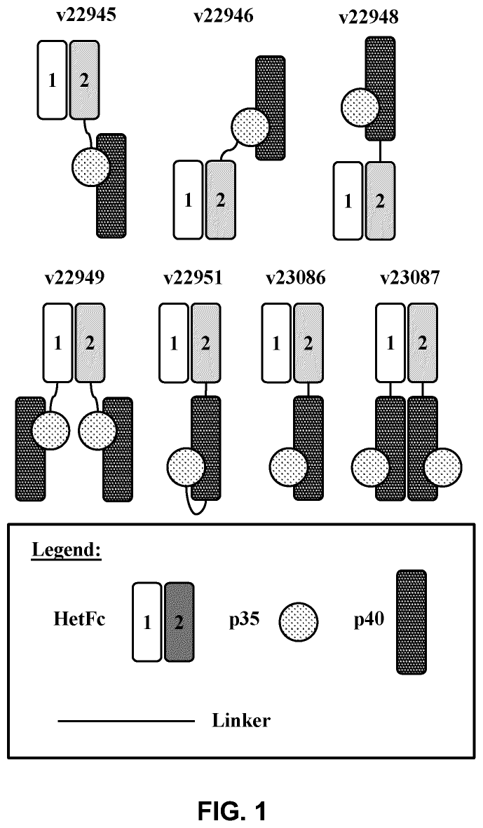

When an IL12 fusion protein described herein comprises a MM and is in the presence of the target (e.g., an IL12 receptor), specific binding of the masked IL12 fusion protein to the IL12 receptor is reduced or inhibited as compared to specific binding of the non-masked parental IL12 fusion protein or the released IL12 polypeptide. As one non-limiting example and as noted elsewhere, in certain embodiments, the masked IL12 fusion protein is in the structural configuration Fc1-L1-MM/Fc2-L2-p40-L3-p35 (see e.g., FIG. 5) wherein at least one of L1, L2, or L3 is a protease cleavable linker. In this setting, where L1 is a cleavable linker, the specific binding of IL12 to its receptor is reduced or inhibited in the uncleaved fusion protein as compared to the specific binding of the fusion protein comprising IL12 after cleavage of L1 by the protease (e.g., as compared to the fusion protein Fc1-L1′/Fc2-L2-p40-L3-p35). Similarly, the specific binding of masked (activatable) IL12 fusion protein to its receptor is reduced or inhibited as compared to the non-masked parent IL12 fusion protein (e.g., Fc1/Fc2-L2-p40-L3-p35 (see e.g., FIG. 1)).

When an IL12 fusion protein described herein comprises a MM and is in the presence of the target (e.g., an IL12 receptor), the potency of the masked IL12 fusion protein is reduced or inhibited as compared to the non-masked parental IL12 fusion protein or the released IL12 polypeptide. Thus, the MM functions to block functional activity of the IL12. As one non-limiting example and as noted elsewhere, in certain embodiments, the masked IL12 fusion protein is in the structural configuration Fc1-L1-MM/Fc2-L2-p40-L3-p35 (see e.g., FIG. 5) wherein at least one of L1, L2, or L3 is a protease cleavable linker. In this setting, where L1 is a cleavable linker, the functional activity or potency of IL12 is reduced when in the uncleaved fusion protein as compared to the potency of the released IL12 after cleavage of L1 by the protease (e.g., as compared to the fusion protein Fc1-L1′/Fc2-L2-p40-L3-p35). Similarly, the potency of the masked (activatable)

IL12 fusion protein is reduced or inhibited as compared to the non-masked parent IL12 fusion protein (e.g., Fc1/Fc2-L2-p40-L3-p35 (see e.g., FIG. 1, FIG. 5)). The reduction of potency of the masked fusion proteins and recovery of cytokine activity after cleavage is described elsewhere herein (see e.g., section above entitled Masked IL12/Protease Activatable IL12 Fusion Proteins).

In certain embodiments, the dissociation constant (K_{d}) of the masked IL12 fusion proteins herein (masked or not) towards an IL12 receptor is generally greater than the K_{d} of the same IL12 fusion protein that does not contain a MM. Conversely, the binding affinity of the masked IL12 fusion proteins towards an IL12 receptor is generally lower than the binding affinity of the IL12 fusion protein not modified with a MM.

In certain embodiments, the K_{d} of the MM towards the IL12 polypeptide is generally greater than the K_{d} of the IL12 polypeptide towards an IL12 receptor. Conversely, in certain embodiments, the binding affinity of the MM towards the IL12 polypeptide is generally lower than the binding affinity of the IL12 polypeptide towards an IL12 receptor.

It should be noted that due to proximity (that is, when the MM is fused by a linker to the IL12 fusion protein), the apparent “affinity” of the MM for the IL12 polypeptide is greater than when the MM is not fused to the IL12 fusion protein.

The MM can inhibit the binding of the masked IL12 fusion protein to the IL12 receptor and thereby inhibit the IL12 functional activity of the fusion protein as compared to the IL12 polypeptide not modified by the MM. The MM can bind to the IL12 polypeptide and inhibit it from binding to its receptor. The MM can sterically inhibit the binding of the masked IL12 fusion protein to the IL12 receptor. The MM can allosterically inhibit the binding of the masked IL12 fusion protein to the IL12 receptor. In those embodiments when the masked IL12 fusion protein is in the presence of the IL12 receptor, there is no binding or substantially no binding of the masked IL12 fusion protein to the IL12 receptor, or no more than 0.001 percent, 0.01 percent, 0.1 percent, 1 percent, 2 percent, 3 percent, 4 percent, 5 percent, 6 percent, 7 percent, 8 percent, 9 percent, 10 percent, 15 percent, 20 percent, 25 percent, 30 percent, 35 percent, 40 percent, or 50 percent binding of the masked IL12 fusion protein to the target, as compared to the binding of the unmasked IL12 fusion protein, the binding of the parental IL12, for at least 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, 12 months or greater when measured in vivo or by Surface Plasmon Resonance (SPR) (see Protocol 12 in the Example section).

In certain embodiments the MM is not a natural binding partner of the IL12 polypeptide. The MM may be a modified binding partner for the IL12 polypeptide which contains amino acid changes that at least slightly decrease affinity and/or avidity of binding to the IL12 polypeptide. In some embodiments the MM contains no or substantially no homology to the IL12 receptor. In other embodiments the MM is no more than 5 percent, 10 percent, 15 percent, 20 percent, 25 percent, 30 percent, 35 percent, 40 percent, 45 percent, 50 percent, 55 percent, 60 percent, 65 percent, 70 percent, 75 percent, or 80 percent similar to an IL12 receptor.

When the IL12 fusion protein is in a ‘masked’ state, even in the presence of the IL12 receptor, the MM interferes with or inhibits the binding of the masked IL12 fusion protein to the receptor. However, in certain embodiments, in the unmasked or cleaved state of the IL12 fusion protein, the MM's interference with target binding to the IL12 receptor is reduced, thereby allowing greater access of the released IL12 polypeptide to its receptor and providing for receptor binding.

For example, when the masked cytokine fusion protein comprises a protease cleavable linker (PCL, see elsewhere herein), the masked cytokine fusion protein can be unmasked upon cleavage of the PCL, in the presence of enzyme, preferably a disease-specific enzyme. Thus, the MM is one that when the masked cytokine fusion protein is uncleaved provides for masking of the cytokine from target binding, but does not substantially or significantly interfere or compete for binding of the cytokine receptor to the released cytokine polypeptide (released when the masked cytokine fusion protein is cleaved). Thus, the combination of the MM and the PCL facilitates the switchable/activatable phenotype, with the MM reducing binding of the cytokine to its receptor when it is in the uncleaved state, and cleavage of the PCL by protease providing for increased binding of target and recovery of the cytokine activity.

The structural properties of the MM will vary according to a variety of factors such as the minimum amino acid sequence required for interference with cytokine binding and/or activity, the cytokine-cytokine receptor protein binding pair of interest, the size of the cytokine and the fusion protein, the length of the PCL, whether the PCL is positioned within the MM, between the Fc and the cytokine, between the Fc and mask, the presence or absence of additional linkers, etc.

The MM can be provided in a variety of different forms. In certain embodiments, the MM can be selected to be a known binding partner of the cytokine. In certain embodiments, the MM is one that masks the cytokine from target binding when the masked cytokine fusion protein is uncleaved but does not substantially or significantly interfere or compete for binding of the target with the cytokine polypeptide that is released after cleavage. In a specific embodiment, the MM do not contain the amino acid sequences of a naturally-occurring binding partner of the cytokine.

The efficiency of the MM to inhibit the binding or activity of the cytokine when coupled can be measured by SPR or a cell based assay as described herein and outlined in detail elsewhere (see e.g., NK, CTLL-2 or CD8+ T cell IFNγ release assays) and as described herein in the Examples section of the disclosure. Masking efficiency of MMs can be determined by at least two parameters: affinity of the MM for the cytokine or a fusion protein comprising the cytokine and the spatial relationship of the MM relative to the binding interface of the cytokine to its receptor.

Regarding affinity, by way of example, a MM may have high affinity but only partially inhibit the binding of the cytokine to its receptor, while another MM may have a lower affinity for the cytokine but fully inhibit target binding. For short time periods, the lower affinity MM may show sufficient masking; in contrast, over time, that same MM may be displaced by the target (due to insufficient affinity for the cytokine).

In a similar fashion, two MA/Is with the same affinity may show different extents of masking based on how well they promote inhibition of the cytokine from binding its receptor. In another example, a MM with high affinity may bind and change the structure of the cytokine or a fusion protein comprising the cytokine so that binding to its target is completely inhibited while another MM with high affinity may only partially inhibit binding. As a consequence, discovery of an effective MM is generally not based only on affinity but can include a measure of the potency of the masked cytokine fusion protein as compared to an appropriate control. Likewise, the effectiveness of the cleavage of the PCL and release of the polypeptide comprising the cytokine can be determined by measuring recovery of cytokine activity post cleavage and is a factor in identifying an effective MM, PCL, and masked cytokine fusion protein configuration.

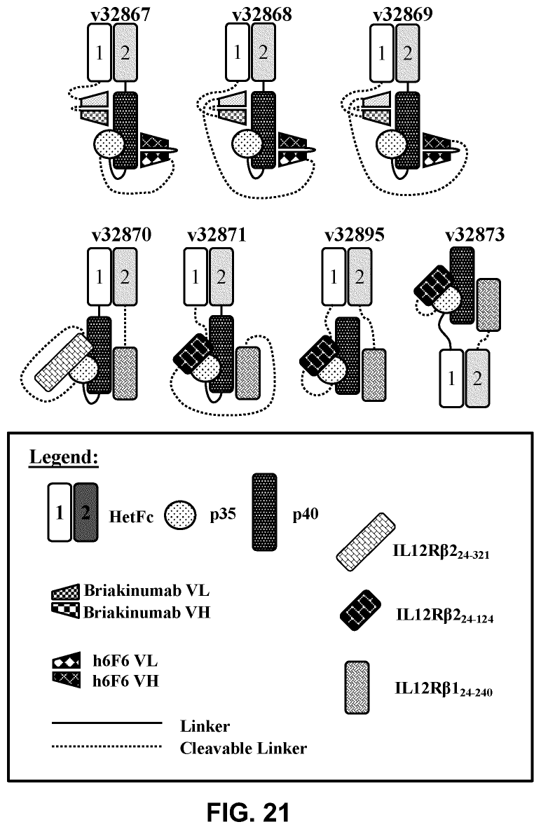

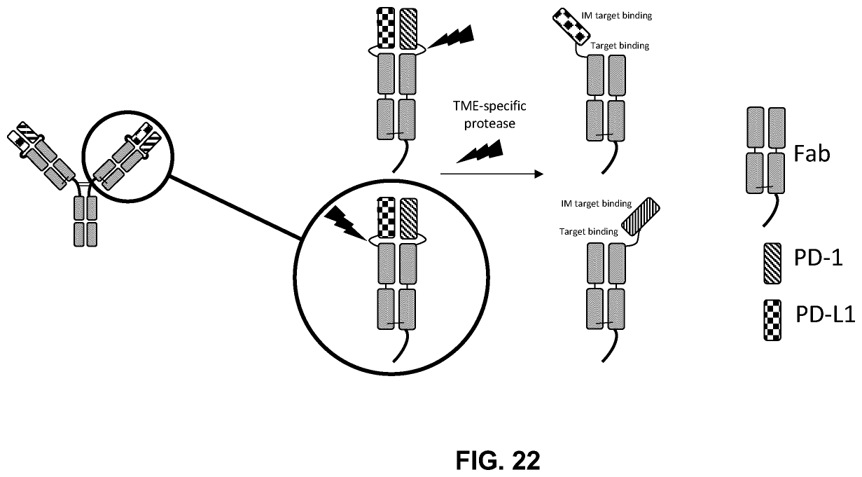

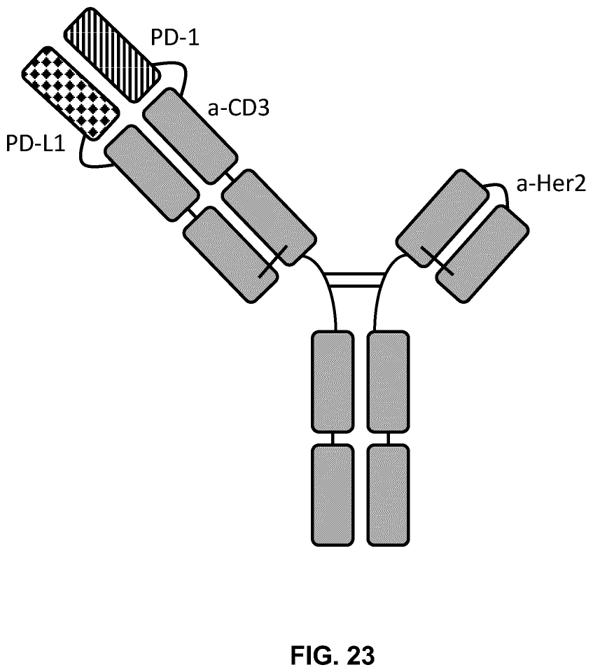

In certain embodiments, a masked cytokine fusion protein may comprise more than one MM (see e.g., FIG. 21, Table 15). In this regard, each MM may be derived from an antibody or antigen-binding fragment thereof or may be derived from a cytokine receptor (e.g., an IL12R) or there may be a combination of MMs derived from antibodies and MMs derived from receptors, or synthetic polypeptide MMs. In one embodiment, a masked cytokine fusion protein herein comprises two MM. In another embodiment, a masked cytokine fusion protein herein comprises two MM wherein one MM is fused via a PCL. In another embodiment, the cytokine fusion protein herein comprises two MM wherein both MMs are fused via a PCL. In one embodiment, one or both MM comprises an additional PCL (e.g., an scFv comprising a PCL between the VH and VL).

The MM may be a single-chain Fv (scFv) antibody fragment, an IL12 receptor β2 subunit (IL12Rβ2) or an IL12-binding fragment thereof, an IL12 receptor 131 subunit (IL12Rβ1) or an IL12-binding fragment thereof (e.g., an extracellular domain (ECD) of the IL12Rβ1), or an IL23R, or an IL23-binding fragment thereof. Illustrative scFv MM comprise the VH and VL amino acid sequences provided in SEQ ID NOs: 11-12 and 255-256, and variants thereof, for example as described in Table 8 (H_Y32A; H_F27V; H_Y52AV; H_R52E; H_R52E_Y52AV; H_H95D; H_G96T; H_H98A; mutations referenced according to Kabat numbering for Briakinumab VH provided in SEQ ID NO:11). In certain embodiments, illustrative MM comprise the VHCDR and VLCDR set forth in SEQ ID NOs:13-18 or the VHCDR and VLCDR set forth in SEQ ID NOs:257-262. In certain embodiments, the MM is an IL12 receptor or an IL12-binding fragment thereof, or variants thereof that retain the ability to block IL12 activity. In one embodiment, the MM is an ECD of human IL12Rβ2, or a variant thereof that blocks IL12 activity. In one particular embodiment, the MM comprises amino acids 24-321 of human IL12Rβ2 (see e.g., amino acids 24-321 of SEQ ID NO:253). In another embodiment, the MM comprises amino acids 24-124 of human Th12Rβ2 (see e.g., amino acids 24-124 of SEQ ID NO:253). In one embodiment, the MM comprises amino acids 24-240 of human IL12Rβ1 (see e.g., amino acids 24-240 of SEQ ID NO:252), or a variant thereof that blocks IL12 activity. In one embodiment, a MM comprises an IL23R ECD (e.g., amino acids 24-355 of SEQ ID NO:263; amino acids 14-318 of SEQ ID NO:263; or amino acids 24-126 of SEQ ID NO:263. See also SEQ ID NOs: 264-266), or a variant thereof that blocks IL23 activity.

Other illustrative MM are described herein and are set forth, for example, in the variants and clones described in the Tables, Examples and sequences provided herein.

### Antibodies and Antigen-Binding Fragments Thereof

In certain embodiments, the masking moieties used in the masked fusion proteins herein comprise an antibody or an antigen-binding fragment of an antibody. Antigen-binding fragments include but are not limited to variable or hypervariable regions of light and/or heavy chains of an antibody (V_{L}, V_{H}), variable fragments (Fv), Fab′ fragments, F(ab′) 2 fragments, Fab fragments, single chain antibodies (scAb), single chain variable regions (scFv), complementarity determining regions (CDR), domain antibodies (dAbs), single domain heavy chain immunoglobulins, single domain light chain immunoglobulins, or other polypeptides known in the art containing an antigen-binding fragment capable of binding target proteins or epitopes on target proteins.

Illustrative antigen-binding domains are derived from antibodies that bind IL12 and/or IL23.

In one embodiment, the MM comprises an antibody or antigen-binding fragment thereof, that specifically binds to IL12. In one embodiment, the MM comprises an antibody or antigen-binding fragment thereof, that specifically binds to IL23. In certain embodiments, the MM comprises an scFv that specifically binds IL12 or IL23.

In some embodiments the MM can be identified through screening antibodies or antigen binding fragments thereof that bind to IL12 or IL23. The candidate MM can be fused in a variety of configurations in a cytokine fusion protein (see for example FIGS. 1, 5-9 and 21 and the Examples herein) and screened for their ability to reduce cytokine binding, reduce IL12 potency and/or for recovery of cytokine activity after cleavage. Antibodies may be derived from antibodies known in the art that bind to IL12 and/or IL23. Such antibodies are known and available for example, from the literature or can be found in the TABS Therapeutic Antibody Database (see tabs(dot)craic(dot)com). Illustrative antibodies for use in the masked IL12 fusion proteins herein include Briakinumab (U.S. Pat. Nos. 6,914,128; 7,504,485; 8,168,760; 8,629,257; 9,035,030); ustekinumab (U.S. Pat. Nos. 6,902,734; 7,279,157; U8080247; U.S. Pat. Nos. 7,736,650; 8,420,081; 7,887,801; 8,361,474; 8,084,233; 9,676,848), AK101, PMA204 (see e.g., U.S. Pat. No. 8,563,697), 6F6 (see e.g., U.S. Pat. No. 8,563,697; Clarke A W et al., 2010 MAbs 2:539-49). The h6F6 antibody binds a different epitope on p40 than Briakinumab or Ustekinumab.

In one embodiment, the MM is derived from an antibody comprising an antigen binding domain that binds to human IL12 and human IL23. In another embodiment, the antibody binds human IL12p40 existing as a monomer (human IL12p40) and as a homodimer (human IL12p80) and the antibody inhibits the binding of human IL12 to human IL12Rβ2 and human IL23 to human IL23R but does not inhibit the binding of human IL12 or human IL23 or human IL12p40 or human IL12p80 to human IL12Rβ1.

Antibodies or antigen binding fragments thereof that bind to IL12 and/or IL23, can be further modified to increase or decrease affinity as needed and then further tested for ability to mask and reduce potency as described herein.

In certain embodiments, candidate peptides can be screened to identify a MM peptide capable of binding IL12 or IL23 using such methods as described for example in WO2010/081173 and U.S. Pat. No. 10,118,961. Such methods comprise, providing a library of peptide scaffolds, wherein each peptide scaffold comprises: a transmembrane Protein™; and a candidate peptide; contacting an IL12 or IL23 with the library; identifying at least one candidate peptide capable of binding the IL12 or IL23 polypeptide; and determining whether the dissociation constant (K_{d}) of the candidate peptide towards the IL12 or IL23 is between 1-10 nM.

### Linkers and Protease Cleavable Linkers

In certain embodiments of the fusion proteins of this disclosure, one or more different components or domains are fused directly one to the other with no linker. For example, in certain embodiments, an Fc domain may be fused directly to a MM or fused directly to a p35 or p40 polypeptide. However, in certain embodiments the masked cytokine fusion constructs comprise one or more linkers of varying lengths. Peptide linkers allow arrangement of the fusion protein to form a functional masking moiety as well as a cytokine that, when cleaved from the larger/full fusion protein, retains cytokine activity. The masked cytokine fusion constructs comprise linkers that comprise protease cleavage sites and also comprise linkers that do not contain cleavage sites.

A “linker” is a peptide that joins or links other peptides or polypeptides, such as a linker of about 2 to about 150 amino acids. In masked cytokine fusion proteins of this disclosure, a linker may be used to fuse any of the components of the fusion protein, such as an Fc polypeptide to a MM or a linker can join an Fc polypeptide to a cytokine polypeptide, e.g., p35 or p40 of IL12. In certain embodiments, a linker may be present within a MM such as where a MM is an scFV and a linker joins the VH and VL.

Exemplary linkers for use in the fusion proteins described herein include those belonging to the (GlynSer) family, such as (Gly3Ser)ₙ(Gly4Ser)₁, (Gly3Ser)i(Gly4Ser)ₙ, (Gly3Ser)ₙ(Gly4Ser)ₙ, or (Gly4Ser)ₙ, wherein n is an integer of 1 to 5. In certain embodiments, the peptide linkers suitable for connecting the different domains include sequences comprising glycine-serine linkers, for example, but not limited to, (GₘS)ₙ-GG, (SGn)m, (SEGn)m, wherein m and n are between 0-20.

In certain embodiments, a linker can be an amino acid sequence obtained, derived, or designed from an antibody hinge region sequence, a sequence linking a binding domain to a receptor, or a sequence linking a binding domain to a cell surface transmembrane region or membrane anchor. In some embodiments, a linker can have at least one cysteine capable of participating in at least one disulfide bond under physiological conditions or other standard peptide conditions (e.g., peptide purification conditions, conditions for peptide storage). In certain embodiments, a linker corresponding or similar to an immunoglobulin hinge peptide retains a cysteine that corresponds to the hinge cysteine disposed toward the amino-terminus of that hinge. In further embodiments, a linker is from an IgG1 hinge and has been modified to remove any cysteine residues or is an IgG1 hinge that has one cysteine or two cysteines corresponding to hinge cysteines.

In addition to providing a spacing function, a linker can provide flexibility or rigidity suitable for properly orienting the one or more domains of the masked cytokine fusion proteins herein, both within the fusion protein and between or among the fusion proteins and their target(s). Further, a linker can support expression of a full-length fusion protein and stability of the purified protein both in vitro and in vivo following administration to a subject in need thereof, such as a human, and is preferably non-immunogenic or poorly immunogenic in those same subjects. In certain embodiments, a linker may comprise part or all of a human immunoglobulin hinge, a stalk region of C-type lectins, a family of type II membrane proteins. Linkers range in length from about 2 to about 100 amino acids, or about 5 to about 75 amino acids, or about 10 to about 50 amino acids, or about 2 to about 40 amino acids, or about 8 to about 20 amino acids, about 10 to about 60 amino acids, about 10 to about 30 amino acids, or about 15 to about 25 amino acids.

In certain embodiments, a linker for use herein may comprise an “altered wild type immunoglobulin hinge region” or “altered immunoglobulin hinge region”. Such altered hinge regions refers to (a) a wild type immunoglobulin hinge region with up to 30 percent amino acid changes (e.g., up to 25 percent, 20 percent, 15 percent, 10 percent, or 5 percent amino acid substitutions or deletions), (b) a portion of a wild type immunoglobulin hinge region that is at least 10 amino acids (e.g., at least 12, 13, 14 or 15 amino acids) in length with up to 30 percent amino acid changes (e.g., up to 25 percent, 20 percent, 15 percent, 10 percent, or 5 percent amino acid substitutions or deletions), or (c) a portion of a wild type immunoglobulin hinge region that comprises the core hinge region (which portion may be 4, 5, 6, 7, 8, 9, 10, 11, 12, 13, 14, or 15, or at least 4, 5, 6, 7, 8, 9, 10, 11, 12, 13, 14, or 15 amino acids in length). In certain embodiments, one or more cysteine residues in a wild type immunoglobulin hinge region, such as an IgG1 hinge comprising the upper and core regions, may be substituted by one or more other amino acid residues (e.g., one or more serine residues). An altered immunoglobulin hinge region may alternatively or additionally have a proline residue of a wild type immunoglobulin hinge region, such as an IgG1 hinge comprising the upper and core regions, substituted by another amino acid residue (e.g., a serine residue).

Alternative hinge and linker sequences that can be used as connecting regions may be crafted from portions of cell surface receptors that connect IgV-like or IgC-like domains. Regions between IgV-like domains where the cell surface receptor contains multiple IgV-like domains in tandem and between IgC-like domains where the cell surface receptor contains multiple tandem IgC-like regions could also be used as connecting regions or linker peptides. In certain embodiments, hinge and linker sequences are from 5 to 60 amino acids long, and may be primarily flexible, but may also provide more rigid characteristics, may contain primarily a helical structure with minimal beta sheet structure.

Certain illustrative linkers are provided in SEQ ID Nos: 240-242. Illustrative linkers are also provided within the context of various masked cytokine and parental non-masked fusion proteins herein as set forth in SEQ ID Nos: 23-89 (see also Table 23).

Where desired, in certain embodiments, the linkers of the masked cytokine fusion proteins herein comprise a protease cleavage site. Where used, the protease cleavage sites are positioned within the linkers so as to maximize recognition and cleavage by the desired protease or proteases and minimize recognition and non-specific cleavage by other proteases. Additionally, the protease cleavage site or sites may be positioned within the linkers (or said differently, may be surrounded by linkers) and are positioned within the fusion protein as a whole so as to achieve the best desired masking and release of the active cytokine post-cleavage.

Accordingly, in certain embodiments, the masked cytokine fusion proteins disclosed herein comprise at least one protease cleavable linker (PCL), when masked and not activated.

As used herein, the PCL of the masked cytokine fusion proteins described herein includes an amino acid sequence that serves as a substrate for at least one protease, usually an extracellular protease, i.e., the PCL comprises one or more cleavage sites, also referred to as cleavage sequences. The polypeptide moiety that is fused to the masked cytokine fusion protein by the PCL and that is released from the masked cytokine fusion protein following cleavage of the PCL can be referred to herein as the cleavable moiety (CM). In certain embodiments, the CM comprises a MM. In another embodiment, the CM comprises the cytokine moiety (e.g., an IL12 or IL23 polypeptide). In certain embodiments, a masked cytokine fusion protein as described herein may comprise more than one CM, e.g., a CM that comprises a MM and a CM that comprises the cytokine polypeptide both of which are released following cleavage by a protease. In certain embodiments where a masked cytokine fusion protein comprises more than one CM, they may be fused to the masked cytokine fusion protein by the same or different PCL, that is having the same cleavage site or different cleavage sites. In this regard, the PCL may also have different linkers.

The cleavage site or cleavage sequence may be selected based on a protease that is co-localized in tissue where the activity of the unmasked (activated) cytokine is desired. A cleavage site can serve as a substrate for multiple proteases, e.g., a substrate for a serine protease and a second different protease, e.g. a matrix metalloproteinase, (an MMP)). In some embodiments, a cleavage site can serve as a substrate for more than one serine protease, e.g., a matriptase and a uPA. In some embodiments, a PCL can serve as a substrate for more than one MMP, e.g., an MMP9 and an MMP 14.

A variety of different conditions are known in which a target of interest (such as a particular tumor type, a particular tumor that expresses a particular tumor associated antigen, a particular tumor type that is infiltrated by immune cells responsive to IL12/23) is co localized with a protease, where the substrate of the protease is known in the art. In the example of cancer, the target tissue can be a cancerous tissue, particularly cancerous tissue of a solid tumor. There are reports in the literature of increased levels of proteases and the presence of innate and adaptive immune cells capable of responding to IL12/23 in a number of cancers, e.g., liquid tumors or solid tumors. See, e.g., La Rocca et al, (2004) British J. of Cancer 90(7): 1414-1421.

Non-limiting examples of disease to be targeted with the masked cytokine fusion proteins herein include: all types of cancers, such as, but not limited to breast, including by way of non-limiting example, triple negative breast cancer, ER/PR+breast cancer, and Her2+ breast cancer, lung cancer (e.g., non-small cell squamous and adenocarcinoma), colorectal cancer, gastric cancer, glioblastoma, ovarian cancer, endometrial cancer, renal cancer, sarcoma, skin cancer, cervical cancer, liver cancer, bladder cancer, cholangiocarcinoma, prostate cancer, melanomas, head and neck cancer (e.g., head and neck squamous cell carcinoma), esophageal, squamous cell cancer, basal cell carcinoma, pancreatic cancer, leukemias, including T-cell acute lymphoblastic leukemia (T-ALL), lymphoblastic diseases including multiple myeloma, and solid tumors. Indications also include bone disease or metastasis in cancer, regardless of primary tumor origin. Other illustrative diseases include rheumatoid arthritis, Crohn's disease, SLE, cardiovascular damage, and ischemia. In certain embodiments, the target disease is selected from the group consisting of colorectal cancer, pancreatic cancer, head and neck cancer, esophageal cancer, bladder cancer, cervical cancer, and lung cancer (e.g., non-small cell squamous and adenocarcinoma).

In certain embodiments, the PCL is specifically cleaved by an enzyme at a rate of about 0.001-1500×10⁴ M⁻¹S⁻¹ or at least 0.001, 0 005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×10⁴M⁻¹S⁻¹.

For specific cleavage by an enzyme, contact between the enzyme and the PCL is made. In certain embodiments, when the masked cytokine (e.g., IL12 or IL23) fusion protein comprises at least a first PCL and is in the presence of sufficient enzyme activity, the PCL can be cleaved. Sufficient enzyme activity can refer to the ability of the enzyme to make contact with the PCL and effect cleavage. It can readily be envisioned that an enzyme may be in the vicinity of the PCL but is unable to cleave because of other cellular factors or protein modification of the enzyme.

In some embodiments, the PCL: has a length of up to 15 amino acids, a length of up to 20 amino acids, a length of up to 25 amino acids, a length of up to 30 amino acids, a length of up to 35 amino acids, a length of up to 40 amino acids, a length of up to 45 amino acids, a length of up to 50 amino acids, a length of up to 60 amino acids, a length in the range of 10-60 amino acids, a length in the range of 15-60 amino acids, a length in the range of 20-60 amino acids, a length in the range of 25-60 amino acids, a length in the range of 30-60 amino acids, a length in the range of 35-60 amino acids, a length in the range of 40-50 amino acids, a length in the range of 45-60 amino acids, a length in the range of 10-40 amino acids, a length in the range of 15-40 amino acids, a length in the range of 20-40 amino acids, a length in the range of 25-40 amino acids, a length in the range of 30-40 amino acids, a length in the range of 35-40 amino acids, a length in the range of 10-30 amino acids, a length in the range of 15-30 amino acids, a length in the range of 20-30 amino acids, a length in the range of 25-30 amino acids, a length in the range of 10-20 amino acids, or a length in the range of 10-15 amino acids.

In certain embodiments, the PCL comprises a protease cleavage recognition site of 6-10 amino acids, or 7-10 amino acids, or 8-10 amino acids in length. In another embodiment, the PCL consists of a protease cleavage recognition site of 6-10 amino acids, or 7-10 amino acids, or 8-10 amino acids in length. In one embodiment, the protease cleavage site is preceded on the N-terminus by a linker sequence of between about 10-20 amino acids, of between 12-16 amino acids, or about 15 amino acids. In another embodiment, the protease cleavage site is followed on the C-terminus by a linker sequence of between about 6-20, 8-15, 8-10, 10-18 amino acids, or in some cases, about 8 amino acids in length. In yet another embodiment, the protease cleavage site is preceded by a linker sequence on the N-terminus and followed by a linker sequence on the C-terminus. Thus, in certain embodiments, the protease cleavage site is situated between two linkers. The linkers on either the N or C-terminal end of the protease cleavage site can be of varying lengths, for example, between about 5-20, 6-20, 8-15, 8-10, 10-18, or 12-16. In certain embodiments the N- or C-terminal linker sequence is about 8 or about 15 amino acids in length.

Exemplary PCLs of the disclosure comprise one or more cleavage sequences recognized by any of a variety of proteases, such as, but not limited to, serine proteases, MMPs (MMP1, MMP2, MMP3, MMP7, MMP8, MMP9, MMP10, MMP11, MMP12, MMP13, MMP14, MMP15, MMP16, MMP17, MMP18 (collagenase 4), MMP19, MMP20, MMP21, etc.), adamalysins, serralysins, astacins, caspases (e.g., caspase 1, caspase 2, caspase 3, caspase 4, caspase 5, caspase 6, caspase 7, caspase 8, caspase 9, caspase 10, caspase 11, caspase 12, caspase 13, caspase 14), cathepsins, (e.g., cathepsin A, cathepsin B, cathepsin D, cathepsin E, cathepsin K, cathepsin S), FAB, granyme B, guanidinobenzoatase (GB), hepsin, elastase, legumain, matriptase 2, meprin, neurosin, MT-SP1, neprilysin, plasmin, PSA, PSMA, TACE, TMPRSS3/4, uPA, and calpain.

In certain embodiments, a PCL may comprise a cleavage sequence that is cleaved by more than one protease. In this regard, a cleavage sequence may be cleaved by 1, 2, 3, 4, 5 or more proteases. In another embodiment, a PCL may comprise a cleavage sequence that is substantially cleaved by one enzyme but not by others. Thus, in some embodiments, a PCL comprises a cleavage sequence that has high specificity. By “high specificity” is meant >90% cleavage observed by a particular protease and less than 50% cleavage observed by other proteases. In certain embodiments, a PCL comprises a cleavage sequence that demonstrates >80% cleavage by one protease but less than 50% cleavage by other proteases. In certain embodiments, a PCL comprises a cleavage sequence that demonstrates >70%, 75%, 76%, 77%, 78%, or 79%, cleavage by one protease but less than 65%, 60%, 55%, 54%, 53%, 52%, 51%, 50%, 49%, 48%, 47%, 46%, or 45% cleavage by other proteases. By way of example, in one embodiment the cleavage sequence may be >90% cleaved by matriptase and ˜75% cleaved by uPa and plasmin. In another embodiment, the cleavage sequence may be cleaved by uPa and matriptase but no specific cleavage by plasmin is observed. In yet another embodiment, the cleavage sequence may be cleaved by uPa and not by matriptase or plasmin. In one embodiment, a cleavage sequence may demonstrate some level of resistance to non-specific protease cleavage (e.g., cleavage by plasmin or other non-specific proteases). In this regard, a protease cleavage sequence may have “high non-specific protease resistance” (<25% cleavage by plasmin or an equivalent non-specific protease), “moderate non-specific protease resistance” (about <75% cleavage by plasmin or an equivalent non-specific protease), or “low non-specific protease resistance” (up to about 90% cleavage by plasmin or an equivalent non-specific protease). In certain embodiments, high non-specific protease resistance is about between <25%-<35% cleavage by plasmin or an equivalent non-specific protease. In some embodiments, moderate non-specific protease resistance is about between <50%-<80% cleavage by plasmin or an equivalent non-specific protease. Such cleavage activity can be measured using assays known in the art, such as by incubation with the appropriate proteases at comparable ratios of enzyme:substrate for all enzymes, followed by SDS-PAGE or other analysis. In certain embodiments, a protease cleavage sequence may display up to complete resistance to protease cleavage to 24 hours contact with protease. In other embodiments, a protease cleavage sequence may display up to complete resistance to non-specific protease cleavage after 0.5 hour-36 hours contact with protease. In another embodiment, a protease cleavage sequence displays up to complete resistance to non-specific protease cleavage after 0.5, 1, 2, 3, 4, 5, 6, 7, 8, 9, 10, 12, 20, 24, 36, 48, or 72 hours contact with an appropriate protease.

Thus, in certain embodiments, the cleavage sequences are selected based on preferences for various desired proteases. In this way, a desired cleavage profile for a particular PCL comprising a cleavage sequence may be selected for a desired purpose (e.g., high specific cleavage in particular tumor microenvironments or specific organs) where a particular protease or set of proteases may demonstrate high, specific, elevated, efficient, moderate, low or no cleavage of a particular cleavage sequence within a PCL. Methods for determining cleavage are known in the art and are described, for example, in Example 2 herein.

In certain embodiments, a PCL may comprise one or more cleavage sequences arranged in tandem, with or without additional linkers in between each cleavage site. In certain embodiments, a PCL comprises a first cleavage sequence and a second cleavage sequence where the first cleavage sequence is cleaved by a first protease and the second cleavage sequence is cleaved by a second protease. As a non-limiting example, a PCL may comprise a first cleavage sequence cleaved by matriptase and uPa and a second cleavage sequence cleaved by an MMP. In certain embodiments, a PCL comprises a first cleavage sequence, a second cleavage sequence and a third cleavage sequence where the first cleavage sequence is cleaved by a first protease, the second cleavage sequence is cleaved by a second protease and the third cleavage sequence is cleaved by a third protease.

Illustrative proteolytic enzymes and their recognition sequences useful in the masked IL12 fusion proteins herein can be identified by one of skill and are known in the art, such as those described in MEROPS database (see e.g., Rawlings, et al. Nucleic Acids Research, Volume 46, Issue D1, 4 Jan. 2018, Pages D624-D632), and elsewhere (Hoadley et al, Cell, 2018; GTEX Consortium, Nature, 2017; Robinson et al, Nature, 2017).

Cleavage sequences may be identified and screened for example, as described in Example 2. Exemplary cleavage sequences include, but are not limited to, those identified in Example 2 and Table 3 herein. Illustrative cleavage sequences for use in the masked cytokine fusion proteins described herein are set forth in SEQ ID Nos:2-10 and 170-239. Other methods may also be used for identifying cleavage sequence for use herein, such as described in U.S. Pat. Nos. 9,453,078, 10,138,272, 9,562,073 and published international application numbers WO 2015/048329; WO2015116933; WO2016118629.

Other illustrative cleavage sequences for use herein are described, for example, in U.S. Pat. Nos. 9,453,078, 10,138,272, 9,562,073 and published international application numbers WO 2015/048329; WO2015116933; WO2016118629. Such cleavage sequences include, for example, LSGRSANP (SEQ ID NO:186), TSGRSANP (SEQ ID NO:2) and LSGRSDNH (SEQ ID NO:3).

Other illustrative cleavage sequences for use herein include the cleavage sequences described in WO2019075405 and WO2016118629, shown in Table 24 and provided in SEQ ID NOs:180-239.

The cleavage sequences described herein and PCLs comprising the cleavage sequences may be used in any of a variety of recombinant proteins where cleavage of a particular moiety from the larger recombinant protein is desired. Such recombinant proteins may comprise two or more domains, such as, but not limited to, the various components or domains described herein, including, but not limited to, a masking moiety, a cytokine such as IL12 or IL23, an antibody or antigen-binding fragment thereof, one or more linkers, an Fc domain, and a targeting domain.

Accordingly, one aspect of the present disclosure provides a recombinant polypeptide that comprises a protease cleavable linker (PCL) wherein the protease cleavable linker comprises one or more of the cleavage sequences set forth herein. In one embodiment, the present disclosure provides a recombinant polypeptide that comprises a PCL wherein the protease cleavable linker comprises the amino acid sequence MSGRSANA (SEQ ID NO:10). In certain embodiments, the recombinant polypeptide comprising a PCL described herein comprises two heterologous polypeptides, a first polypeptide located amino (N) terminally to the PCL and a second polypeptide located carboxyl (C) terminally to the PCL, the two heterologous polypeptides thus separated by the PCL.

In one embodiment, the two heterologous polypeptides are selected from a cytokine polypeptide or functional fragment thereof, an antibody, an antigen-binding fragment of an antibody and an Fc domain. In another embodiment, the recombinant polypeptide comprises a cytokine polypeptide or a functional fragment thereof, a MM, and an Fc domain. In certain other embodiments, the MM is a single-chain Fv (scFv) antibody fragment that binds to the cytokine or a cytokine receptor polypeptide or a cytokine-binding fragment thereof. In a further embodiment, the recombinant polypeptide comprises an antibody or antigen binding fragment thereof that binds a target, and a MM that binds to the antibody or antigen binding fragment thereof and blocks binding of the antibody or antigen binding fragment thereof to the target.

In one embodiment, the present disclosure provides an isolated recombinant polypeptide comprising a PCL, wherein the PCL comprises the amino acid sequence MSGRSANA as set forth in SEQ ID NO: 10, wherein the PCL comprises a substrate for a protease (protease cleavage site), wherein the isolated recombinant polypeptide comprises at least one moiety (M) selected from the group consisting of a moiety that is located amino (N) terminally to the PCL (MN), a moiety that is located carboxyl (C) terminally to the PCL (MC), and combinations thereof, and wherein the MN or MC is selected from the group consisting of an antibody or antigen binding fragment thereof; a cytokine or a functional fragment thereof; a MM (as described in more detail elsewhere herein); a cytokine receptor or a functional fragment thereof; an immunomodulatory receptor, or functional fragment thereof; an immune checkpoint protein or a functional fragment thereof; a tumor associated antigen; a targeting domain; a therapeutic agent; an antineoplastic agent; a toxic agent; a drug; and a detectable label.

### Fc Domains

In some embodiments, the masked IL12 fusion proteins described herein comprise an Fc, and in some embodiments, the Fc is a dimeric Fc.

The term “Fc domain” or “Fc region” herein is used to define a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, Md., 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence.

An Fc domain comprises either a CH3 domain or a CH3 and a CH2 domain. The CH3 domain comprises two CH3 sequences, one from each of the two Fc polypeptides of the dimeric Fc. The CH2 domain comprises two CH2 sequences, one from each of the two Fc polypeptides of the dimeric Fc.

In some aspects, the Fc comprises at least one or two CH3 sequences. In some aspects, the Fc is coupled, with or without one or more linkers, to a first antigen-binding polypeptide construct and/or a second antigen-binding polypeptide construct. In some aspects, the Fc is a human Fc. In some aspects, the Fc is a human IgG or IgG1 Fc. In some aspects, the Fc is a heterodimeric Fc. In some aspects, the Fc comprises at least one or two CH2 sequences.

In some aspects, the Fc comprises one or more modifications in at least one of the CH3 sequences. In some aspects, the Fc comprises one or more modifications in at least one of the CH2 sequences. In some aspects, an Fc is a single polypeptide. In some aspects, an Fc is multiple peptides, e.g., two polypeptides.

In some aspects, an Fc is an Fc described in patent applications PCT/CA2011/001238, filed Nov. 4, 2011 (WO2012058768; U.S. Pat. Nos. 9,562,109 and 10,875,931) or PCT/CA2012/050780, filed Nov. 2, 2012 (WO2013063702); U.S. Pat. Nos. 9,574,010; 9,732,155; 10,457,742 and US Pat. Application No.: US2020008741), all of which are herein incorporated by reference in their entirety.

### Modified CH3 Domains

In some aspects, the masked IL12 fusion proteins described herein comprises a heterodimeric Fc (“HetFc”) comprising a modified CH3 domain that has been asymmetrically modified. The heterodimeric Fc can comprise two heavy chain constant domain polypeptides: a first Fc polypeptide and a second Fc polypeptide, which can be used interchangeably provided that the Fc domain comprises one first Fc polypeptide and one second Fc polypeptide. Generally, the first Fc polypeptide comprises a first CH3 sequence and the second Fc polypeptide comprises a second CH3 sequence. In certain diagrams and elsewhere herein, a first Fc polypeptide and a second Fc polypeptide may be referred to as Fc polypeptide A and Fc polypeptide B (or chain A or chain B as shorthand), which similarly can be used interchangeably provided that the Fc domain or region comprises one Fc polypeptide A and one Fc polypeptide B. In some cases, the Fc domain which comprises one Fc polypeptide A and one Fc polypeptide B may be referred to as a variant and the variant may be referred to by a particular variant number to distinguish it from other Fc variants.

Two CH3 sequences that comprise one or more amino acid modifications introduced in an asymmetric fashion generally results in a heterodimeric Fc, rather than a homodimer, when the two CH3 sequences dimerize. As used herein, “asymmetric amino acid modifications” refers to any modification where an amino acid at a specific position on a first CH3 sequence is different from the amino acid on a second CH3 sequence at the same position, and the first and second CH3 sequence preferentially pair to form a heterodimer, rather than a homodimer. This heterodimerization can be a result of modification of only one of the two amino acids at the same respective amino acid position on each sequence; or modification of both amino acids on each sequence at the same respective position on each of the first and second CH3 sequences. The first and second CH3 sequence of a heterodimeric Fc can comprise one or more than one asymmetric amino acid modification.

Table C provides the amino acid sequence of the human IgG1 Fc sequence, corresponding to amino acids 231 to 447 of the full-length human IgG1 heavy chain. The CH3 sequence comprises amino acid 341-447 of the full-length human IgG1 heavy chain.

Typically, an Fc can include two contiguous heavy chain sequences (A and B) that are capable of dimerizing. In some aspects, one or both sequences of an Fc include one or more mutations or modifications at the following locations: L351, F405, Y407, T366, K392, T394, T350, S400, and/or N390, using EU numbering. In some aspects, an Fc includes a variant sequence shown in Table 2. In some aspects, an Fc includes the mutations of Variant 1 A-B. In some aspects, an Fc includes the mutations of Variant 2 A-B. In some aspects, an Fc includes the mutations of Variant 3 A-B. In some aspects, an Fc includes the mutations of Variant 4 A-B. In some aspects, an Fc includes the mutations of Variant 5 A-B.

TABLE C
IgG1 Fc sequences

| Human IgG1 | APELLGGPSVFLFPPK | |
|---|---|---|
| Fc sequence | PKDTLMISRTPEVTC | |
| 231-447 | VVVDVSHEDPEVKFN | |
| (EU-numbering) | WYVDGVEVHNAKTKP | |
| | REEQYNSTYRVVSVL | |
| | TVLHQDWLNGKEYKC | |
| | KVSNKALPAPIEKTI | |
| | SKAKGQPREPQVYTL | |
| | PPSRDELTKNQVSLT | |
| | CLVKGFYPSDIAVEW | |
| | ESNGQPENNYKTTPP | |
| | VLDSDGSFFLYSKLT | |
| | VDKSRWQQGNVFSCS | |
| | VMHEALHNHYTQKSL | |
| | SLSPGK | |
| Variant | | |
| IgG1 Fc | | |
| sequence | | |
| (231-447) | Chain | Mutations |
| 1 | A | L351Y_F405A_Y407V |
| 1 | B | T366LK392M_T394W |
| 2 | A | L351Y_F405A_Y407V |
| 2 | B | T366L_K392L_T394W |
| 3 | A | T350V_L351Y_F405A_Y407V |
| 3 | B | T350V_T366L_K392L_T394W |
| 4 | A | T350V_L351Y_F405A_Y407V |
| 4 | B | T350V_T366L_K392M_T394W |
| 5 | A | T350V_L351Y_S400E_ |
| | | F405A_Y407V |
| 5 | B | T350V_T366L_N390R_ |
| | | K392M_T394W |

The first and second CH3 sequences can comprise amino acid mutations as described herein, with reference to amino acids 231 to 447 of the full-length human IgG1 heavy chain. In one embodiment, the heterodimeric Fc comprises a modified CH3 domain with a first CH3 sequence having amino acid modifications at positions F405 and Y407, and a second CH3 sequence having amino acid modifications at position T394. In one embodiment, the heterodimeric Fc comprises a modified CH3 domain with a first CH3 sequence having one or more amino acid modifications selected from L351Y, F405A, and Y407V, and the second CH3 sequence having one or more amino acid modifications selected from T366L, T366I, K392L, K392M, and T394W.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, and one of the first or second CH3 sequences further comprising amino acid modifications at position Q347, and the other CH3 sequence further comprising amino acid modification at position K360. In another embodiment, a heterodimeric Fc comprises a modified CH3 domain with a first CH3 sequence having amino acid modifications at positions L351, F405 and Y407, and a second CH3 sequence having amino acid modifications at position T366, K392, and T394, one of the first or second CH3 sequences further comprising amino acid modifications at position Q347, and the other CH3 sequence further comprising amino acid modification at position K360, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394 and one of said first and second CH3 sequences further comprising amino acid modification of D399R or D399K and the other CH3 sequence comprising one or more of T411E, T411D, K409E, K409D, K392E and K392D. In another embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, one of said first and second CH3 sequences further comprises amino acid modification of D399R or D399K and the other CH3 sequence comprising one or more of T411E, T411D, K409E, K409D, K392E and K392D, and one or both of said CH3 sequences further comprise the amino acid modification T350V.

In one embodiment, a heterodimeric Fc comprises a modified CH3 domain with a first CH3 sequence having amino acid modifications at positions L351, F405 and Y407, and a second CH3 sequence having amino acid modifications at positions T366, K392, and T394, wherein one or both of said CH3 sequences further comprise the amino acid modification of T350V.

In one embodiment, a heterodimeric Fc comprises a modified CH3 domain comprising the following amino acid modifications, where “A” represents the amino acid modifications to the first CH3 sequence, and “B” represents the amino acid modifications to the second CH3 sequence: A:L351Y_F405A_Y407V, B:T366L_K392M_T394W, A:L351Y_F405A_Y407V, B:T366L_K392L_T394W, A:T350V_L351Y_F405A_Y407V, B:T350V_T366L_K392L_T394W, A:T350V_L351Y_F405A_Y407V, B:T350V_T366L_K392M_T394W, A:T350V_L351Y_S400E_F405A_Y407V, and/or B:T350V_T366L_N390R_K392M_T394W.

The one or more asymmetric amino acid modifications can promote the formation of a heterodimeric Fc in which the heterodimeric CH3 domain has a stability that is comparable to a wild-type homodimeric CH3 domain. In an embodiment, the one or more asymmetric amino acid modifications promote the formation of a heterodimeric Fc domain in which the heterodimeric Fc domain has a stability that is comparable to a wild-type homodimeric Fc domain. In an embodiment, the one or more asymmetric amino acid modifications promote the formation of a heterodimeric Fc domain in which the heterodimeric Fc domain has a stability observed via the melting temperature (Tm) in a differential scanning calorimetry study, and where the melting temperature is within 4° C. of that observed for the corresponding symmetric wild-type homodimeric Fc domain. In some aspects, the Fc comprises one or more modifications in at least one of the CH3 sequences that promote the formation of a heterodimeric Fc with stability comparable to a wild-type homodimeric Fc.

### Modified CH2 Domains

In certain embodiments, an Fc domain contemplated for use herein is an Fc having a modified CH2 domain. In some embodiments, an Fc domain contemplated for use herein is an IgG Fc having a modified CH2 domain, wherein the modification of the CH2 domain results in altered binding to one or more Fc receptors (FcRs) such as receptors of the FcγRI, FcγRII and FcγRIII subclasses.

A number of amino acid modifications to the CH2 domain that selectively alter the affinity of the Fc for different Fcγ receptors are known in the art. Amino acid modifications that result in increased binding and amino acid modifications that result in decreased binding can both be useful in certain indications. For example, increasing binding affinity of an Fc for FcγRIIIa (an activating receptor) results in increased antibody dependent cell-mediated cytotoxicity (ADCC), which in turn results in increased lysis of the target cell. Decreased binding to FcγRIIb (an inhibitory receptor) likewise may be beneficial in some circumstances. In certain indications, a decrease in, or elimination of, ADCC and complement-mediated cytotoxicity (CDC) may be desirable. In such cases, modified CH2 domains comprising amino acid modifications that result in increased binding to FcγRIIb or amino acid modifications that decrease or eliminate binding of the Fc region to all of the Fcγ receptors (“knock-out” variants) may be useful.

Examples of amino acid modifications to the CH2 domain that alter binding of the Fc by Fcγ receptors include, but are not limited to, the following: S298A/E333A/K334A and S298A/E333A/K334A/K326A (increased affinity for FcγRIIIa) (Lu, et al., 2011, J Immunol Methods, 365(1-2):132-41); F243L/R292P/Y300L/V305I/P396L (increased affinity for FcγRIIIa) (Stavenhagen, et al., 2007, Cancer Res, 67(18):8882-90); F243L/R292P/Y300L/L235V/P396L (increased affinity for FcγRIIIa) (Nordstrom J L, et al., 2011, Breast Cancer Res, 13(6):R123); F243L (increased affinity for FcγRIIIa) (Stewart, et al., 2011, Protein Eng Des Sel., 24(9):671-8); S298A/E333A/K334A (increased affinity for FcγRIIIa) (Shields, et al., 2001, J Biol Chem, 276(9):6591-604); S239D/I332E/A330L and S239D/I332E (increased affinity for FcγRIIIa) (Lazar, et al., 2006, Proc Natl Acad Sci USA, 103(11):4005-10), and S239D/S267E and S267E/L328F (increased affinity for FcγRIIb) (Chu, et al., 2008, Mol Immunol, 45(15):3926-33).

Additional modifications that affect Fc binding to Fcγ receptors are described in Therapeutic Antibody Engineering (Strohl & Strohl, Woodhead Publishing series in Biomedicine No 11, ISBN 1 907568 37 9, October 2012, page 283).

In certain embodiments, a masked IL12 fusion protein comprises a scaffold based on an IgG Fc having a modified CH2 domain, in which the modified CH2 domain comprises one or more amino acid modifications that result in decreased or eliminated binding of the Fc region to all of the Fcγ receptors (i.e. a “knock-out” variant).

Various publications describe strategies that have been used to engineer antibodies to produce “knock-out” variants (see, for example, Strohl, 2009, Curr Opin Biotech 20:685-691, and Strohl & Strohl, “Antibody Fc engineering for optimal antibody performance” In Therapeutic Antibody Engineering, Cambridge: Woodhead Publishing, 2012, pp 225-249). These strategies include reduction of effector function through modification of glycosylation (described in more detail below), use of IgG2/IgG4 scaffolds, or the introduction of mutations in the hinge or CH2 domain of the Fc (see also, U.S. Patent Publication No. 2011/0212087, International Publication No. WO 2006/105338, U.S. Patent Publication No. 2012/0225058, U.S. Patent Publication No. 2012/0251531 and Strop et al., 2012, J. Mol. Biol., 420: 204-219).

Specific, non-limiting examples of known amino acid modifications to reduce FcγR and/or complement binding to the Fc include those identified in Table D.

TABLE D
Modifications to Reduce Fcγ Receptor
or Complement Binding to the Fc

| Company | Mutations |
|---|---|
| GSK | N297A |
| Ortho Biotech | L234A/L235A |
| Protein Design labs | IgG2 V234A/G237A |
| Wellcome Labs | IgG4 L235A/G237A/E318A |
| GSK | IgG4 S228P/L236E |
| Merck | IgG2 H268Q/V309L/A330S/A331S |
| Bristol-Myers | C220S/C226S/C229S/P238S |
| Seattle Genetics | C226S/C229S/E3233P/L235V/L235A |
| Medimmune | L234F/L235E/P331S |

Additional examples include Fc regions engineered to include the amino acid modifications L234A/L235A/D265S. In addition, asymmetric amino acid modifications in the CH2 domain that decrease binding of the Fc to all Fcγ receptors are described in International Publication No. WO 2014/190441.

In additional embodiments, certain amino acid substitutions are introduced into human IgG1 Fc for Fc domain of the present disclosure to ablate immune effector functions such as antibody-dependent cell cytotoxicity (ADCC) and complement-dependent cytotoxicity (CDC). Mutations in the CH2 region of the antibody heavy chains may include positions 234, 235, and 265 in EU numbering to reduce or eliminate immune effector functions.

### Targeting Domain

In certain embodiments, the masked IL12 fusion proteins described herein may comprise a “targeting domain” that targets the fusion proteins to a site of action (e.g. sites of inflammation, a particular anatomical site such as an organ, or to a tumor). As used herein, the “targeted antigen” is the antigen recognized and specifically bound by the targeting domain.

In some embodiments, the targeting domain is specific for (specifically binds) an antigen found on cells in a protease-rich environment such as the tumor microenvironment. In some embodiments, the encoded targeting domain is specific for (e.g., specifically binds or recognizes) regulatory T cells (Tregs), for example targeting the CCR4 or CD39 receptors. Other suitable targeting domains comprise those that have a cognate ligand that is overexpressed in inflamed tissues, e.g., the IL1 receptor, or the IL6 receptor. In other embodiments, a suitable targeting domain is one that has a cognate ligand present on an immune cell such as a dendritic cell (DC), a T cell, an NK cell, etc. In other embodiments, the suitable targeting domain comprise those that have a cognate ligand that is overexpressed in tumor tissue, e.g., a tumor-associated antigen (TAA).

TAAs contemplated herein for tumor targeting include but are not limited to EpCAM, EGFR, HER-2, HER-3, c-Met, FOLR1, and CEA. In certain embodiments, the masked fusion proteins comprise two targeting domains that bind to two different target antigens known to be expressed on a diseased cell or tissue. Exemplary pairs of antigen binding domains include but are not limited to EGFR/CEA, EpCAM/CEA, and HER-2/HER-3.

Suitable targeting domains include antigen-binding domains, such as antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like. Other suitable antigen-binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of antigen-binding polypeptides include a ligand for a desired receptor, a ligand-binding portion of a receptor, a lectin, and peptides that binds to or associates with one or more target antigens.

In some embodiments, a targeting domain specifically binds to a cell surface molecule. In some embodiments, a targeting domain specifically binds to a tumor antigen. In some embodiments, the targeting domain specifically and independently binds to a tumor antigen selected from at least one of Fibroblast activation protein alpha (FAPa), Trophoblast glycoprotein (5T4), Tumor-associated calcium signal transducer 2 (Trop2), Fibronectin EDB (EDB-FN), fibronectin F.IIIB domain, CGS-2, EpCAM, EGER, HER-2, HER-3, cMet, CEA, and FOLR1. In some embodiments, the targeting polypeptides specifically and independently bind to two different antigens, wherein at least one of the antigens is a tumor antigen selected from EpCAM, EGFR, HER-2, HER-3, cMet, CEA, and FOLR1. The TAA targeted by the targeting domain can be a tumor antigen expressed on a tumor cell. Tumor antigens are well known in the art and include, for example, EpCAM, EGFR, HER-2, HER-3, c-Met, FOLR1, PSMA, CD38, BCMA, and CEA. 5T4, AFP, B7-H3, Cadherin-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Mud, Muc16, NaPi2b, Nectin-4, P-cadherin, NY-ESO-1, PRLR, PSCA, PTK7, ROR1, SLC44A4, SLTRK5, SLTRK6, STEAP1, TIM1, Trop2, WT1.

In some embodiments, the targeted antigen is an immune checkpoint protein. Examples of immune checkpoint proteins include but are not limited to CD27, CD137, 2B4, TIGIT, CD155, ICOS, HVEM, CD40L, LIGHT, TIM-1, X40, DNAM-1, PD-L1, PD1, PD-L2, CTLA-4, CD80, CD40, CEACAM1, CD48, CD70, A2AR, CD39, CD73, B7-H3, B7-H4, BTLA, IDO1, ID02, TDO, KIR, LAG-3, TIM-3, or VISTA. In certain embodiments, the targeting domain is an antibody or antigen-binding fragment thereof that specifically binds to an immune checkpoint protein or the targeting domain is a ligand that binds to an immune checkpoint protein or is a binding fragment thereof.

The targeting domain can specifically bind to a cell surface molecule such as a protein, lipid or polysaccharide. In some embodiments, a targeted antigen is an antigen expressed on a tumor cell, virally infected cell, bacterially infected cell, damaged red blood cell, arterial plaque cell, inflamed or fibrotic tissue cell. The targeted antigen can comprise an immune response modulator. Examples of immune response modulator include but are not limited to granulocyte-macrophage colony stimulating factor (GM-CSF), macrophage colony stimulating factor (M-CSF), granulocyte colony stimulating factor (G-CSF), interleukin 2 (IL2), interleukin 3 (IL3), interleukin 12 (IL12), interleukin 15 (IL15), B7-1 (CD80), B7-2 (CD86), GITRL, CD3, or GITR.

In certain embodiments, the targeting domain specifically binds a cytokine receptor. Examples of cytokine receptors include, but are not limited to, Type I cytokine receptors, such as GM-CSF receptor, G-CSF receptor, Type I IL receptors, Epo receptor, LIF receptor, CNTF receptor, TPO receptor; Type II Cytokine receptors, such as IFN-alpha receptor (IFNAR1, IFNAR2), IFB-beta receptor, IFN-gamma receptor (IFNGR1, IFNGR2), Type II IF receptors; chemokine receptors, such as CC chemokine receptors, CXC chemokine receptors, CX3C chemokine receptors, XC chemokine receptors; tumor necrosis receptor superfamily receptors, such as TNFRSF5/CD40, TNFRSF8/CD30, TNFRSF7/CD27, TNFRSF1A/TNFRUCD120a, TNFRSF1B/TNFR2/CD120b; TGF-beta receptors, such as TGF-beta receptor 1, TGF-beta receptor 2; Ig super family receptors, such as IF-1 receptors, CSF-1R, PDGFR (PDGFRA, PDGFRB), SCFR.

In some embodiments, the targeting domain is fused to the masked IL12 fusion protein via a linker or a PCL. In certain embodiments, the linker fusing the targeting domain to the masked IL12 fusion protein is a PCL which is cleaved at the site of action (e.g. by inflammation or cancer specific proteases). In this regard, the PCL may be the same as or different from any other PCL that is present in the masked IL12 fusion protein, such as a PCL fusing a MM to an Fc polypeptide, a PCL present with the MM or a PCL that links an IL12 polypeptide to an Fc polypeptide. In certain embodiments, the PCL fusing the targeting domain is the same as a PCL fusing the MM to an Fc polypeptide and/or the PCL fusing the IL12 to an Fc polypeptide whereby, all of the cleavage sites are cleaved upon reaching the target. In some embodiments, the targeting domain is fused to the masked IL12 fusion protein via a linker which is not cleaved at the site of action (e.g. by inflammation or cancer specific proteases).

### Polypeptides and Polynucleotides

The masked cytokine (e.g., IL12 and other members of the IL12 family of cytokines) fusion proteins described herein comprise at least one polypeptide. Also described are polynucleotides encoding the polypeptides described herein. The masked cytokine fusion proteins are typically isolated.

As used herein, “isolated” means an agent (e.g., a polypeptide or polynucleotide) that has been identified and separated and/or recovered from a component of its natural cell culture environment. Contaminant components of its natural environment are materials that would interfere with diagnostic or therapeutic uses for the masked cytokine fusion proteins, and may include enzymes, hormones, and other proteinaceous or non-proteinaceous solutes. Isolated also refers to an agent that has been synthetically produced, e.g., via human intervention.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full length proteins, wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, etc.; and chemically synthesized compounds having properties known in the art to be characteristic of amino acids. Examples of non-naturally occurring amino acids include, but are not limited to, α-methyl amino acids (e.g. α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the proteins described herein may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Also provided herein are polynucleotides encoding the masked cytokine fusion proteins. The term “polynucleotide” or “nucleotide sequence” is intended to indicate a consecutive stretch of two or more nucleotide molecules. The nucleotide sequence may be of genomic, cDNA, RNA, semisynthetic or synthetic origin, or any combination thereof.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

“Conservatively modified variants” applies to both amino acid and nucleic acid sequences. With respect to particular nucleic acid sequences, “conservatively modified variants” refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of ordinary skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the deletion of an amino acid, addition of an amino acid, or substitution of an amino acid with a chemically similar amino acid. Conservative substitution tables providing functionally similar amino acids are known to those of ordinary skill in the art. Such conservatively modified variants are in addition to and do not exclude polymorphic variants, interspecies homologs, and alleles described herein.

Conservative substitution tables providing functionally similar amino acids are known to those of ordinary skill in the art. The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and [0139] 8) Cysteine (C), Methionine (M).

The terms “identical” or percent “identity,” in the context of two or more nucleic acids or polypeptide sequences, refer to two or more sequences or subsequences that are the same. Sequences are “substantially identical” if they have a percentage of amino acid residues or nucleotides that are the same (i.e., about 60% identity, about 65%, about 70%, about 75%, about 80%, about 85%, about 90%, about 95%, about 96%, about 97%, about 98%, or about 99% identity over a specified region), when compared and aligned for maximum correspondence over a comparison window, or designated region as measured using one of the following sequence comparison algorithms (or other algorithms available to persons of ordinary skill in the art) or by manual alignment and visual inspection. This definition also refers to the complement of a test sequence. The identity can exist over a region that is at least about 50 amino acids or nucleotides in length, or over a region that is 75-100 amino acids or nucleotides in length, or, where not specified, across the entire sequence of a polynucleotide or polypeptide. A polynucleotide encoding a polypeptide described herein, including homologs from species other than human, may be obtained by a process comprising the steps of screening a library under stringent hybridization conditions with a labeled probe having a polynucleotide sequence described herein or a fragment thereof, and isolating full-length cDNA and genomic clones containing said polynucleotide sequence. Such hybridization techniques are well known to the skilled artisan.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known to those of ordinary skill in the art. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of an algorithm that is suitable for determining percent sequence identity and sequence similarity are the BLAST and BLAST 2.0 algorithms, which are described in Altschul et al. (1997) Nuc. Acids Res. 25:3389-3402, and Altschul et al. (1990) J. Mol. Biol. 215:403-410, respectively. Software for performing BLAST analyses is publicly available through the National Center for Biotechnology Information available at the World Wide Web at ncbi.nlm.nih.gov. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1992) Proc. Natl. Acad. Sci. USA 89:10915) alignments (B) of 50, expectation (E) of 10, M=5, N=−4, and a comparison of both strands. The BLAST algorithm is typically performed with the “low complexity” filter turned off

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001.

The phrase “selectively (or specifically) hybridizes to” refers to the binding, duplexing, or hybridizing of a molecule only to a particular nucleotide sequence under stringent hybridization conditions when that sequence is present in a complex mixture (including but not limited to, total cellular or library DNA or RNA).

The phrase “stringent hybridization conditions” refers to hybridization of sequences of DNA, RNA, or other nucleic acids, or combinations thereof under conditions of low ionic strength and high temperature as is known in the art. Typically, under stringent conditions a probe will hybridize to its target subsequence in a complex mixture of nucleic acid (including but not limited to, total cellular or library DNA or RNA) but does not hybridize to other sequences in the complex mixture. Stringent conditions are sequence-dependent and will be different in different circumstances. Longer sequences hybridize specifically at higher temperatures. An extensive guide to the hybridization of nucleic acids is found in Tijssen, Laboratory Techniques in Biochemistry and Molecular Biology-Hybridization with Nucleic Probes, “Overview of principles of hybridization and the strategy of nucleic acid assays” (1993).

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. The engineered proteins are expressed and produced by standard molecular biology techniques.

By “isolated nucleic acid molecule or polynucleotide” is intended a nucleic acid molecule, DNA or RNA, which has been removed from its native environment. For example, a recombinant polynucleotide encoding a polypeptide contained in a vector is considered isolated. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extra-chromosomally or at a chromosomal location that is different from its natural chromosomal location. Isolated RNA molecules include in vivo or in vitro RNA transcripts, as well as positive and negative strand forms, and double-stranded forms. Isolated polynucleotides or nucleic acids described herein, further include such molecules produced synthetically, e.g., via PCR or chemical synthesis. In addition, a polynucleotide or a nucleic acid, in certain embodiments, include a regulatory element such as a promoter, ribosome binding site, or a transcription terminator.

The term “polymerase chain reaction” or “PCR” generally refers to a method for amplification of a desired nucleotide sequence in vitro, as described, for example, in U.S. Pat. No. 4,683,195. In general, the PCR method involves repeated cycles of primer extension synthesis, using oligonucleotide primers capable of hybridising preferentially to a template nucleic acid.

By a nucleic acid or polynucleotide having a nucleotide sequence at least, for example, 95% “identical” to a reference nucleotide sequence of the present disclosure, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present disclosure can be determined conventionally using known computer programs, such as the ones discussed above for polypeptides (e.g. ALIGN-2).

A derivative, or a variant of a polypeptide is said to share “homology” or be “homologous” with the peptide if the amino acid sequences of the derivative or variant has at least 50% identity with a 100 amino acid sequence from the original peptide. In certain embodiments, the derivative or variant is at least 75% the same as that of either the peptide or a fragment of the peptide having the same number of amino acid residues as the derivative. In certain embodiments, the derivative or variant is at least 85% the same as that of either the peptide or a fragment of the peptide having the same number of amino acid residues as the derivative. In certain embodiments, the amino acid sequence of the derivative is at least 90% the same as the peptide or a fragment of the peptide having the same number of amino acid residues as the derivative. In some embodiments, the amino acid sequence of the derivative is at least 95%, 96%, 97%, or 98% the same as the peptide or a fragment of the peptide having the same number of amino acid residues as the derivative. In certain embodiments, the derivative or variant is at least 99% the same as that of either the peptide or a fragment of the peptide having the same number of amino acid residues as the derivative.

The term “modified,” as used herein refers to any changes made to a given polypeptide, such as changes to the length of the polypeptide, the amino acid sequence, chemical structure, co-translational modification, or post-translational modification of a polypeptide. The form “(modified)” term means that the polypeptides being discussed are optionally modified, that is, the polypeptides under discussion can be modified or unmodified.

In some aspects, a masked cytokine fusion protein construct comprises an amino acid sequence that is at least 80, 85, 90, 91, 92, 93, 94, 95, 96, 97, 98, 99, or 100% identical to a relevant amino acid sequence or fragment thereof set forth in the Table(s) or accession number(s) disclosed herein. In some aspects, a masked cytokine fusion protein comprises an amino acid sequence encoded by a polynucleotide that is at least 80, 85, 90, 91, 92, 93, 94, 95, 96, 97, 98, 99, or 100% identical to a relevant nucleotide sequence or fragment thereof set forth in the Table(s) or accession number(s) disclosed herein.

### Methods of Preparing Masked IL12 Fusion Proteins/Recombinant Proteins

The masked IL12 fusion proteins or other recombinant proteins (e.g., recombinant proteins comprising a PCL) described herein may be produced using standard recombinant methods known in the art (see, e.g., U.S. Pat. No. 4,816,567 and “Antibodies: A Laboratory Manual,” 2nd Edition, Ed. Greenfield, Cold Spring Harbor Laboratory Press, New York, 2014) and as further outlined herein.

Typically, for recombinant production of a masked IL12 fusion proteins or other recombinant proteins, nucleic acid encoding the masked IL12 fusion proteins or other recombinant proteins is isolated and inserted into one or more vectors for further cloning and/or expression in a host cell. Such nucleic acid may be readily isolated and sequenced using conventional procedures (e.g. by using oligonucleotide probes that are capable of binding specifically to genes masked IL12 fusion proteins or other recombinant proteins).

Suitable host cells for cloning or expression of masked IL12 fusion proteins or other recombinant proteins encoding vectors include prokaryotic or eukaryotic cells described herein.

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

As used herein, the term “eukaryote” refers to organisms belonging to the phylogenetic domain Eucarya such as animals (including but not limited to, mammals, insects, reptiles and birds), ciliates, plants (including but not limited to, monocots, dicots and algae), fungi, yeasts, flagellates, microsporidia, protists, and the like.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, and the like) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, and the like) phylogenetic domain.

For example, a masked IL12 fusion protein construct or other recombinant protein comprising a PCL construct described herein may be produced in bacteria, in particular when glycosylation and Fc effector function are not needed. For expression of polypeptides in bacteria, see, for example, U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the masked IL12 fusion protein or other recombinant protein as described herein may be isolated from the bacterial cell paste in a soluble fraction and can be further purified.

In addition to prokaryotes, eukaryotic microbes such as filamentous fungi or yeast are suitable cloning or expression hosts for multi-specific antigen-binding construct-encoding vectors, including fungi and yeast strains whose glycosylation pathways have been “humanized,” resulting in the production of an antigen-binding construct with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing recombinant proteins, in particular antigen-binding constructs, in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol., 36:59 (1977)); baby hamster kidney cells (BHK); mouse sertoli cells (TM4 cells as described, e.g., in Mather, Biol Reprod, 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumour (MMT 060562); TRI cells, as described, e.g., in Mather et al., Annals N.Y. Acad Sci, 383:44-68 (1982); MRC 5 cells; and FS4 cells. Other useful mammalian host cell lines include Chinese hamster ovary (CHO) cells, including DHFR—CHO cells (Urlaub et al., Proc Natl Acad Sci USA, 77:4216 (1980)); and myeloma cell lines such as YO, NSO and Sp2/0. For a review of certain mammalian host cell lines suitable for antigen-binding construct production, see, e.g., Yazaki & Wu, Methods in Molecular Biology, Vol. 248 (B.K.C. Lo, ed., Humana Press, Totowa, N.J.), pp. 255-268 (2003).

In some embodiments, the masked IL12 fusion proteins or other recombinant proteins described herein are produced in stable mammalian cells by a method comprising transfecting at least one stable mammalian cell with nucleic acid encoding the masked IL12 fusion protein or other recombinant protein described herein, in a predetermined ratio, and expressing the nucleic acid in the at least one mammalian cell. In some embodiments, the predetermined ratio of nucleic acid is determined in transient transfection experiments to determine the relative ratio of input nucleic acids that results in the highest percentage of the fusion proteins in the expressed product (see also Example section for Protocols 3 and 4 and Example 3).

In some embodiments, in the method of producing a masked IL12 fusion protein or other recombinant protein described herein, in stable mammalian cells, the expression product of the stable mammalian cell comprises a larger percentage of the desired masked HetFc IL12 fusion protein as compared to the monomeric fusion protein. In certain embodiments, the fusion proteins herein are glycosylated.

In some embodiments, in the method of producing a fusion protein in stable mammalian cells, the method further comprises identifying and purifying the desired fusion protein. In some embodiments, identification is by one or both of liquid chromatography and mass spectrometry (see also the Examples herein).

If required, the masked IL12 fusion proteins or other recombinant proteins can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can be used for purification of antigen-binding constructs. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni+2 affinity chromatography if a His-tag is employed, or immobilized anti-flag antibody if a flag-tag is used. For general guidance in suitable purification techniques, see, e.g., Protein Purification: Principles and Practice, 3rd Ed., Scopes, Springer-Verlag, N.Y. (1994). The degree of purification necessary will vary depending on the use of the antigen-binding constructs. In some instances, no purification may be necessary.

In certain embodiments, the masked IL12 fusion proteins or other recombinant proteins may be purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q or DEAE columns, or their equivalents or comparables.

In some embodiments, the masked IL12 fusion proteins or other recombinant proteins may be purified using Cation Exchange Chromatography including, but not limited to, chromatography on SP-sepharose, CM sepharose, poros HS, poros CM, Toyopearl SP, Toyopearl CM, Resource/Source S or CM, or Fractogel S or CM columns, or their equivalents or comparables.

In certain embodiments, the masked IL12 fusion proteins or other recombinant proteins herein are substantially pure. The term “substantially pure” (or “substantially purified”) refers to a construct described herein, or variant thereof, that may be substantially or essentially free of components that normally accompany or interact with the protein as found in its naturally occurring environment, i.e. a native cell, or host cell in the case of recombinantly produced construct. In certain embodiments, a construct that is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein. When the construct is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the construct is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less.

In certain embodiments, the term “substantially purified” as applied to a masked HetFc IL12 fusion protein comprising a heterodimeric Fc as described herein means that the heterodimeric Fc has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, size-exclusion chromatography (SEC) and capillary electrophoresis.

The masked IL12 fusion proteins and other recombinant proteins may also be chemically synthesized using techniques known in the art (see, e.g., Creighton, Proteins: Structures and Molecular Principles, W. H. Freeman & Co., N.Y. (1983), and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4-diaminobutyric acid, alpha-amino isobutyric acid, 4aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as α-methyl amino acids, C α-methyl amino acids, N α-methyl amino acids, and amino acid analogs in general. Furthermore, the amino acid can be D (dextrorotary) or L (levorotary)

Certain embodiments of the present disclosure relate to isolated nucleic acid encoding a masked HetFc IL12 fusion protein or other recombinant protein described herein. Such nucleic acid may encode an amino acid sequence comprising the VL and/or an amino acid sequence comprising the VH of the MM, or a modified IL12 polypeptide, etc.

Certain embodiments relate to vectors (e.g. expression vectors) comprising nucleic acid encoding a masked HetFc IL12 fusion protein or other recombinant protein described herein. The nucleic acid may be comprised by a single vector or it may be comprised by more than one vector. In some embodiments, the nucleic acid is comprised by a multicistronic vector.

Certain embodiments relate to host cells comprising such nucleic acid or one or more vectors comprising the nucleic acid. In some embodiments, a host cell comprises (e.g. has been transformed with) a vector comprising a nucleic acid that encodes an amino acid sequence comprising a first fusion protein as described herein (e.g., a first Fc polypeptide fused to a MM etc.) and an amino acid sequence comprising a second fusion protein as described herein (e.g., a second Fc polypeptide fused to an IL12 or IL23 polypeptide). In some embodiments, a host cell comprises (e.g. has been transformed with) a first vector comprising a nucleic acid that encodes an amino acid sequence comprising a first fusion protein as described herein (e.g., a first Fc polypeptide fused to a MM) and a second vector comprising a nucleic acid that encodes an amino acid sequence comprising a second fusion protein as described herein (e.g., a second Fc polypeptide fused to an IL12 or IL23 polypeptide). In some embodiments, the host cell is eukaryotic, e.g. a Chinese Hamster Ovary (CHO) cell, or human embryonic kidney (HEK) cell, or lymphoid cell (e.g. YO, NSO, Sp20 cell).

Certain embodiments relate to a method of making a masked IL12 fusion protein by culturing a host cell into which nucleic acid encoding the fusion protein has been introduced, under conditions suitable for expression of the masked IL12 fusion protein, and optionally recovering the masked IL12 fusion protein from the host cell (or host cell culture medium).

### Post-Translational Modifications

In certain embodiments, the masked IL12 fusion proteins described herein may be differentially modified during or after translation.

The term “modified,” as used herein, refers to any changes made to a given polypeptide, such as changes to the length of the polypeptide, the amino acid sequence, chemical structure, co-translational modification, or post-translational modification of a polypeptide.

The term “post-translationally modified” refers to any modification of a natural or non-natural amino acid that occurs to such an amino acid after it has been incorporated into a polypeptide chain. The term encompasses, by way of example only, co-translational in vivo modifications, co-translational in vitro modifications (such as in a cell-free translation system), post-translational in vivo modifications, and post-translational in vitro modifications.

In some embodiments, the masked IL12 fusion proteins may comprise a modification such as glycosylation, acetylation, phosphorylation, amidation, derivatization by known protecting/blocking groups, proteolytic cleavage or linkage to an antibody molecule or antigen-binding construct or other cellular ligand, or a combination of these modifications. In some embodiments, the masked IL12 fusion proteins may be chemically modified by known techniques including, but not limited to, specific chemical cleavage by cyanogen bromide, trypsin, chymotrypsin, papain, V8 protease or NaBH4; acetylation; formylation; oxidation; reduction or metabolic synthesis in the presence of tunicamycin.

Additional optional post-translational modifications of masked IL12 fusion proteins or portions thereof, terminal ends, attachment of chemical moieties to the amino acid backbone, chemical modifications of N-linked or O-linked carbohydrate chains, and addition or deletion of an N-terminal methionine residue as a result of prokaryotic host cell expression. The masked IL12 fusion proteins described herein may optionally be modified with a detectable label, such as an enzymatic, fluorescent, isotopic or affinity label to allow for detection and isolation of the protein. Examples of suitable enzyme labels include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin or aequorin; and examples of suitable radioactive materials include iodine, carbon, sulfur, tritium, indium, technetium, thallium, gallium, palladium, molybdenum, xenon or fluorine.

In some embodiments, the masked IL12 fusion proteins described herein may be attached to macrocyclic chelators that associate with radiometal ions.

In those embodiments in which the masked IL12 fusion proteins are modified, either by natural processes, such as post-translational processing, or by chemical modification techniques, the same type of modification may optionally be present in the same or varying degrees at several sites in a given polypeptide. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination (see, e.g., Proteins-Structure and Molecular Properties, 2nd Ed., T. E. Creighton, W. H. Freeman and Company, New York (1993); Post-Translational Covalent Modification of Proteins, B. C. Johnson, Ed., Academic Press, New York, pgs. 1-12 (1983); Seifter et al., Meth. Enzymol. 182:626-646 (1990); Rattan et al., Ann. N.Y. Acad. Sci. 663:48-62 (1992)).

In certain embodiments, the masked IL12 fusion proteins may be attached to a solid support, which may be particularly useful for immunoassays or purification of polypeptides that are bound by, or bind to, or associate with proteins described herein. Such solid supports include, but are not limited to, glass, cellulose, polyacrylamide, nylon, polystyrene, polyvinyl chloride or polypropylene.

### Pharmaceutical Compositions

Also provided herein are pharmaceutical compositions comprising a masked IL12 fusion protein described herein. Pharmaceutical compositions comprise the masked IL12 fusion protein and a pharmaceutically acceptable carrier.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. In some aspects, the carrier is a man-made carrier not found in nature. Water can be used as a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the bispecific anti-HER2 antigen-binding construct, preferably in purified form, together with a suitable amount of carrier so as to provide the form for proper administration to the patient. The formulation should suit the mode of administration.

In certain embodiments, the composition comprising a masked IL12 fusion protein is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the compositions described herein are formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxide isopropylamine, triethylamine, 2-ethylamino ethanol, histidine, procaine, etc.

### Methods of Use

The present disclosure provides methods of using the masked IL12 fusion proteins and other recombinant fusion proteins comprising the PCL described herein.

In particular, further provided herein are methods of treating a subject with or at risk of developing cancer, autoimmune disease, inflammatory disorders or an infectious disease. Further provided herein are methods of treating a subject with or at risk of developing a disease selected from the group consisting of: all types of cancers, such as, but not limited to breast, including by way of non-limiting example, triple negative breast cancer, ER/PR+breast cancer, and Her2+ breast cancer, lung cancer (e.g., non-small cell squamous and adenocarcinoma), colorectal cancer, gastric cancer, glioblastoma, ovarian cancer, endometrial cancer, renal cancer, sarcoma, skin cancer, cervical cancer, liver cancer, bladder cancer, cholangiocarcinoma, prostate cancer, melanomas, head and neck cancer (e.g., head and neck squamous cell carcinoma), esophageal, squamous cell cancer, basal cell carcinoma, pancreatic cancer, leukemias, including T-cell acute lymphoblastic leukemia (T-ALL), lymphoblastic diseases including multiple myeloma, solid tumors, bone disease or metastasis in cancer, regardless of primary tumor origin. Further provided are methods of treating a subject with or at risk of developing rheumatoid arthritis, Crohn's disease, SLE, cardiovascular damage, or ischemia.

In certain embodiments, the present disclosure provides methods of treating a disease in a subject by administering to the subject a therapeutically effective amount of a masked cytokine fusion protein disclosed herein where the disease is selected from the group consisting of colorectal cancer, pancreatic cancer, head and neck cancer, esophageal cancer, bladder cancer, cervical cancer, and lung cancer (e.g., non-small cell squamous and adenocarcinoma).

The methods comprise administering to the subject in need thereof an effective amount of a masked IL12 fusion protein or other recombinant fusion protein as described herein (e.g., comprising a PCL) (a fusion protein) as disclosed herein that is typically administered as a pharmaceutical composition. In some embodiments, the method further comprises selecting a subject with or at risk of developing cancer. In some embodiments, the pharmaceutical composition comprises a masked IL12 fusion protein, or a fragment thereof that is activated at a tumor site. In one embodiment, the tumor is a solid tumor.

In certain embodiments, provided is a method of treating a cancer comprising administering to a subject in which such treatment, prevention or amelioration is desired, a masked IL12 fusion protein described herein, in an amount effective to treat, prevent or ameliorate the cancer. In other embodiments, there is provided a method of using the masked IL12 fusion protein described herein in the preparation of a medicament for the treatment, prevention, or amelioration of cancer in a subject.

The term “subject” refers to an animal, in some embodiments a mammal, which is the object of treatment, observation or experiment. An animal may be a human, a non-human primate, a companion animal (e.g., dogs, cats, and the like), farm animal (e.g., cows, sheep, pigs, horses, and the like) or a laboratory animal (e.g., rats, mice, guinea pigs, and the like).

The term “mammal” as used herein includes but is not limited to humans, non-human primates, canines, felines, murines, bovines, equines, and porcines.

“Treatment” refers to clinical intervention in an attempt to alter the natural course of the individual or cell being treated and can be performed either for prophylaxis or during the course of clinical pathology. Desirable effects of treatment include preventing occurrence or recurrence of disease, alleviation of symptoms, diminishing of any direct or indirect pathological consequences of the disease, preventing metastasis, decreasing the rate of disease progression, amelioration or palliation of the disease state, and remission or improved prognosis. In some embodiments, masked IL12 fusion protein described herein are used to delay development of a disease or disorder. In one embodiment, masked IL12 fusion protein described herein and methods described herein effect tumor regression. In one embodiment, masked IL12 fusion protein described herein and methods described herein effect inhibition of tumor/cancer growth.

Desirable effects of treatment include, but are not limited to, one or more of preventing occurrence or recurrence of disease, alleviation of symptoms, diminishment of any direct or indirect pathological consequences of the disease, preventing metastasis, decreasing the rate of disease progression, amelioration or palliation of the disease state, improved survival, and remission or improved prognosis. In some embodiments, masked IL12 fusion protein described herein are used to delay development of a disease or to slow the progression of a disease.

The term “effective amount” as used herein refers to that amount of a masked IL12 fusion protein described herein or a composition comprising a masked IL12 fusion protein described herein being administered, which will accomplish the goal of the recited method, e.g., relieve to some extent one or more of the symptoms of the disease, condition or disorder being treated. The amount of the composition described herein which will be effective in the treatment, inhibition and prevention of a disease or disorder associated with aberrant expression and/or activity of a therapeutic protein can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

The masked IL12 fusion protein described herein is administered to a subject. Various delivery systems are known and can be used to administer a masked IL12 fusion protein formulation described herein, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intratumoral, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, in certain embodiments, it is desirable to introduce the masked IL12 fusion protein compositions described herein into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it is desirable to administer the masked IL12 fusion proteins described herein, or compositions described herein, locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including a masked IL12 fusion protein described herein, care must be taken to use materials to which the protein does not absorb.

In another embodiment, the masked IL12 fusion proteins described herein or composition comprising same can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, a masked IL12 fusion protein described herein or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol.

Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, e.g., the brain, thus requiring only a fraction of the systemic dose (see, e.g., Goodson, in Medical Applications of Controlled Release, vol. 2, pp. 115-138 (1984)).

In a specific embodiment comprising a nucleic acid encoding a masked IL12 fusion protein described herein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The masked IL12 fusion proteins described herein may be administered alone or in combination with other types of treatments (e.g., radiation therapy, chemotherapy, hormonal therapy, immunotherapy, immune checkpoint inhibitors, and anti-tumor agents). Generally, administration of products of a species origin or species reactivity (in the case of antibodies) that is the same species as that of the patient is preferred.

The masked IL12 fusion proteins described herein may be used in the treatment of cancer. In some embodiments, the masked IL12 fusion proteins described herein may be used in the treatment of a patient who has undergone one or more alternate forms of anti-cancer therapy. In some embodiments, the patient has relapsed or failed to respond to one or more alternate forms of anti-cancer therapy. In other embodiments, a masked IL12 fusion protein is administered to a patient in combination with one or more alternate forms of anti-cancer therapy. In other embodiments, the masked IL12 fusion protein is administered to a patient that has become refractory to treatment with one or more alternate forms of anti-cancer therapy.

### Kits and Articles of Manufacture

Also described herein are kits comprising one or more masked IL12 fusion protein or other recombinant protein described herein. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the masked IL12 fusion proteins.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilized form and the kit can additionally contain a suitable solvent for reconstitution of the lyophilized components. Irrespective of the number or type of containers, the kits described herein also may comprise an instrument for assisting with the administration of the composition to a patient. Such an instrument may be an inhalant, nasal spray device, syringe, pipette, forceps, measured spoon, eye dropper or similar medically approved delivery vehicle.

Certain embodiments relate to an article of manufacture containing materials useful for treatment of a patient as described herein. The article of manufacture comprises a container and a label or package insert on or associated with the container. Suitable containers include, for example, bottles, vials, syringes, intravenous solution bags, etc. The containers may be formed from a variety of materials such as glass or plastic. The container holds a composition comprising the masked IL12 fusion protein which is by itself or combined with another composition effective for treating the patient and may have a sterile access port (for example the container may be an intravenous solution bag or a vial having a stopper pierceable by a hypodermic injection needle). The label or package insert indicates that the composition is used for treating the condition of choice. In some embodiments, the article of manufacture may comprise (a) a first container with a composition contained therein, wherein the composition comprises a masked IL12 fusion protein described herein; and (b) a second container with a composition contained therein, wherein the composition in the second container comprises a further cytotoxic or otherwise therapeutic agent. In such embodiments, the article of manufacture may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. The article of manufacture may optionally further include other materials desirable from a commercial and user standpoint, including other buffers, diluents, filters, needles, and syringes.

### Exemplary Embodiments

Further particular embodiments of the present disclosure are described as follows.

These embodiments are intended to illustrate the compositions and methods described in the present disclosure and are not intended to limit the scope of the present disclosure.

1. A masked interleukin 12 (IL12) fusion protein, comprising an Fc domain comprising a first Fc polypeptide and a second Fc polypeptide; a masking moiety (MM); and an IL12 polypeptide; wherein the masking moiety is fused to the first Fc polypeptide by a first linker; and optionally, wherein the masking moiety further comprises a second linker; wherein the IL12 polypeptide is fused to the second Fc polypeptide by a third linker; wherein at least one of the first, second or third linkers is protease cleavable; and wherein the IL12 activity of the masked IL12 fusion protein is attenuated as compared to the IL12 activity of the IL12 containing polypeptide released after cleavage of the at least one protease cleavable linker.

2. The masked IL12 fusion protein of embodiment 1, wherein the first linker is protease cleavable and optionally, the second linker is protease cleavable. 3. The masked IL12 fusion protein of embodiment 1, wherein the third linker is protease cleavable and optionally, the first or second linker is protease cleavable, or both. 4. The masked IL12 fusion protein of embodiment 1, wherein the first linker comprises a cleavage sequence selected from the group consisting of the cleavage sites listed in Table 3 and Table 24. 5. The masked IL12 fusion protein of embodiment 1, wherein the first linker comprises a cleavage sequence having the amino acid sequence MSGRSANA (SEQ ID NO:10). 6. The masked IL12 fusion protein of embodiment 1, wherein the protease cleavable linker is cleaved by a protease selected from the group consisting of a matrix metalloproteinase (MMP), a matriptase, a cathepsin, a kallikrein, a caspase, a serine protease, and an elastase. 7. The masked IL12 fusion protein of embodiment 1 wherein the first, second and third linkers are cleaved by the same protease.

8. The masked IL12 fusion protein of embodiment 1, wherein the masking moiety is a single-chain Fv (scFv) antibody fragment, an IL12 receptor β2 subunit (IL12Rβ2) or an IL12-binding fragment thereof, or an IL12 receptor 131 subunit (IL12Rβ1) or an IL12-binding fragment thereof 9. The masked IL12 fusion protein of embodiment 8, wherein the scFv comprises the VHCDR1-3 having the amino acid sequences set forth in SEQ ID NO:β-15, respectively and the VLCDR1-3 having the amino acid sequence set forth in SEQ ID NO: 16-18, respectively. 10. The masked IL12 fusion protein of embodiment 8, wherein the scFv comprises a VH and VL comprising the amino acid sequence set forth in SEQ ID NOs:11 and 12, respectively; or a VH and VL comprising the amino acid sequence set forth in SEQ ID NOs:255 and 256, respectively. 11. The masked IL12 fusion protein of embodiment 8, wherein the scFv comprises a variant of the VH having the amino acid sequence set forth in SEQ ID NO:11 wherein the variant is selected from the group consisting of H_Y32A; H_F27V; H_Y52AV; H_R52E; H_R52E_Y52AV; H_H95D; H_G96T; and H_H98A, according to Kabat numbering; and the VL having the amino acid sequence set forth in SEQ ID NO: 12. 12. The masked IL12 fusion protein of embodiment 8, wherein the masking moiety is selected from an ECD of human IL12Rβ2, amino acids 24-321 of human Th12Rβ2 (IL12Rβ224-321), amino acids 24-124 of human Th12Rβ2 (IL12Rβ24-124), amino acids 24-240 of human IL12Rβ1 (IL12Rβ124-240) and an IL23R ECD.

13. The masked IL12 fusion protein of embodiment 1, wherein the IL12 polypeptide comprises the amino acid sequence set forth in SEQ ID NO:22 or 23. 14. The masked IL12 fusion protein of embodiment 13, wherein the IL12 polypeptide comprises the p40 polypeptide amino acid sequence set forth in SEQ ID NO:22 and the p35 IL12 polypeptide is non-covalently bound to the p40 polypeptide. 15. The masked IL12 fusion protein of embodiment 13, wherein the IL12 polypeptide comprises the p35 polypeptide amino acid sequence set forth in SEQ ID NO:23 and the p40 IL12 polypeptide is non-covalently bound to the p40 polypeptide.

16. The masked IL12 fusion protein of embodiment 1, wherein the IL12 polypeptide is a single chain IL12 polypeptide selected from a single chain IL12 polypeptide having the orientation p35-linker-p40 or p40-linker-p35. 17. The masked IL12 fusion protein of embodiment 16, wherein the fusion protein is selected from variants 29243, 29244, 31277, 32039, 32042, 32045, and 32454. 18. The masked IL12 fusion protein of embodiment 16, wherein the single chain IL12 polypeptide is a p40-linker-p35 polypeptide that is fused to the second Fc polypeptide at the p40 polypeptide. 19. The masked IL12 fusion protein of embodiment 16, wherein the single chain IL12 polypeptide is a p35-linker-p40 polypeptide that is fused to the second Fc polypeptide at the p35 polypeptide. 20. The masked IL12 fusion protein of embodiment 18 or embodiment 19, wherein the single chain IL12 polypeptide is fused to the c-terminal end of the second Fc polypeptide. 21. The masked IL12 fusion protein of embodiment 18 or embodiment 19, wherein the single chain IL12 polypeptide is fused to the c-terminal end of the second Fc polypeptide and the masking moiety is fused to the c-terminal end of the first Fc polypeptide. 22. The masked IL12 fusion protein of embodiment 18 or embodiment 19, wherein the single chain IL12 polypeptide is fused to the second Fc polypeptide and wherein the third linker is protease cleavable. 23. The masked IL12 fusion protein of embodiment 18 or embodiment 19, wherein the P40 domain of the IL12 polypeptide has been modified to be more resistant to proteolytic cleavage as compared to an unmodified P40 domain. 24. The masked IL12 fusion protein of embodiment 20, wherein the masking moiety is a single-chain Fv (scFv) antibody fragment; and wherein the IL12 fusion protein further comprises a second masking moiety comprising an additional scFv fused by a fourth linker to the p35 domain of the IL12 polypeptide. 25. The masked IL12 fusion protein of embodiment 24, wherein the first and fourth linkers are protease cleavable. 26. The masked IL12 fusion protein of embodiment 20, wherein the masking moiety comprises a first scFv fused to a second scFv by a fourth linker. 27. The masked IL12 fusion protein of embodiment 26, wherein the first and fourth linkers are protease cleavable. 28. The masked IL12 fusion protein of embodiment 27, wherein the masking moiety is in the following orientation: first Fc polypeptide-L1-VH-VL-L4-VH-VL; or first Fc polypeptide-L1-VH-VL-L4-VL-VH. 29. The masked IL12 fusion protein of embodiment 28, wherein the first and fourth linkers are protease cleavable.

30. The masked IL12 fusion protein of embodiment 1, wherein the masking moiety comprises an IL12 receptor β2 subunit (IL2Rβ2) or an IL12-binding fragment thereof, and an IL12 receptor 131 subunit (IL2Rβ1) or an IL12-binding fragment thereof, fused by the second linker. 31. The masked IL12 fusion protein of embodiment 30, wherein the masking moiety comprises an IL12Rβ2-Ig domain fused to the c-terminal end of the first Fc polypeptide and the IL12Rβ1 fused by the second linker to the c-terminal end of the IL12Rβ2-Ig domain. 32. The masked IL12 fusion protein of embodiment 31, wherein the first and the second linker are protease cleavable.

33. The masked IL12 fusion protein of embodiment 20, wherein the masking moiety is an IL12Rβ1 or an IL12-binding fragment thereof; and wherein the IL12 fusion protein further comprises a second masking moiety comprising an IL12Rβ2 or an IL12-binding fragment thereof fused by a fourth linker to the p35 domain of the IL12 polypeptide. 34. The masked IL12 fusion protein of embodiment 33, wherein the first and the fourth linker are protease cleavable. 35. The masked IL12 fusion protein of embodiment 1 further comprising a targeting domain. 36. The masked IL12 fusion protein of embodiment 35 wherein the targeting domain specifically binds a tumor-associated antigen.

37. The masked IL12 fusion protein of embodiment 1 wherein the first Fc polypeptide comprises a first CH3 domain and the second Fc polypeptide comprises a second CH3 domain. 38. The masked IL12 fusion protein of embodiment 1 wherein the IL12 activity is determined by measuring relative cell abundance or cytokine production of a cell or a cell line that is sensitive to IL12. 39. The masked IL12 fusion protein of embodiment 38 wherein the cell or cell line is selected from PBMC, CD8+ T cells, a CTLL-2 cell line and an NK cell line. 40. The masked IL12 fusion protein of embodiment 38 wherein the IL12 activity is determined by measuring IFNγ release by CD8+ T cells. 41. The masked IL12 fusion protein of embodiment 38 wherein the IL12 activity is determined by measuring the relative cell abundance of NK cells. 42. The masked IL12 fusion protein of embodiment 36 wherein the first CH3 domain or the second CH3 domain or both comprise an asymmetric amino acid modification wherein the first and second CH3 domain preferentially pair to form a heterodimer rather than a homodimer.

43. A masked interleukin 12 (IL12) fusion protein, comprising: an Fc domain comprising a first Fc polypeptide and a second Fc polypeptide; a masking moiety (MM); and an IL12 polypeptide; wherein the masking moiety is fused to the first Fc polypeptide by a first linker; and optionally, wherein the masking moiety further comprises a second linker; wherein the IL12 polypeptide is fused to the second Fc polypeptide by a third linker; optionally, wherein at least one of the first, second or third linkers is protease cleavable; and wherein the IL12 activity of the masked IL12 fusion protein is attenuated as compared to the IL12 activity of a control IL12 polypeptide.

44. A masked IL12 fusion protein, comprising: an Fc domain comprising a first Fc polypeptide and a second Fc polypeptide; a first MM and a second MM; and an IL12 polypeptide; wherein the IL12 polypeptide comprises a p35 polypeptide and a p40 polypeptide; wherein the first MM is fused to the first Fc polypeptide by a first linker; wherein the p35 polypeptide is fused to the first MM by a second linker; wherein the second MM is fused to the second Fc polypeptide by a third linker; and wherein the p40 polypeptide is non-covalently bound to the p35 polypeptide; and wherein at least one of the first, second or third linkers is protease cleavable; and wherein the IL12 activity of the masked IL12 fusion protein is attenuated as compared to the IL12 activity of the IL12 containing polypeptide released after cleavage of the at least one protease cleavable linker.

45. A masked IL12 fusion protein, comprising: an Fc domain comprising a first Fc polypeptide and a second Fc polypeptide; a first MM and a second MM; and an IL12 polypeptide; wherein the IL12 polypeptide comprises a p35 polypeptide and a p40 polypeptide; wherein the p35 polypeptide is fused to the first Fc polypeptide by a first linker; wherein the first MM is fused to the p35 polypeptide by a second linker; wherein the second MM is fused to the second Fc polypeptide by a third linker; and wherein the p40 polypeptide is non-covalently bound to the p35 polypeptide; and wherein at least one of the first, second or third linkers is protease cleavable; and wherein the IL12 activity of the masked IL12 fusion protein is attenuated as compared to the IL12 activity of the IL12 containing polypeptide released after cleavage of the at least one protease cleavable linker.

46. The masked IL12 fusion protein of embodiment 43 wherein the first MM is fused to the C-terminal end of the first Fc polypeptide and wherein the second MM is fused to the C-terminal end of the second Fc polypeptide. 47. The masked IL12 fusion protein of embodiment 45 wherein the p35 polypeptide is fused to the N-terminal end of the first Fc polypeptide and wherein the second MM is fused to the N-terminal end of the second Fc polypeptide. 48. A composition comprising the masked IL12 fusion protein of any one of embodiments 1 to 47 and a pharmaceutically acceptable excipient. 49. An isolated nucleic acid encoding the masked IL12 fusion protein of any one of embodiments 1 to 47. 50. An expression vector comprising the isolated nucleic acid of embodiment 49. 51. A host cell comprising the isolated nucleic acid of embodiment 49 or the expression vector of embodiment 50. 52. A method of making a masked IL12 fusion protein comprising culturing the host cell of embodiment 51 under conditions suitable for expression of the masked IL12 fusion protein and optionally, recovering the masked IL12 fusion protein from the host cell culture medium. 53. A method of treating cancer in a subject comprising administering to the subject a therapeutically effective amount of the composition of embodiment 48.

54. A masked interleukin 23 (IL23) fusion protein, comprising: an Fc domain comprising a first Fc polypeptide and a second Fc polypeptide; a masking moiety; a first protease cleavable linker; and an IL23 polypeptide; wherein the masking moiety is fused to the first Fc polypeptide by the first protease cleavable linker; and optionally, wherein the masking moiety further comprises a second protease cleavable linker; wherein the IL23 polypeptide is fused to the second Fc polypeptide; and wherein the IL23 activity of the masked IL23 fusion protein is attenuated as compared to the IL23 activity of the IL23 containing polypeptide released after cleavage of the protease cleavable linker. 55. The masked IL23 fusion protein of embodiment 54, wherein the IL23 is a single chain IL23 polypeptide selected from a single chain IL23 polypeptide having the orientation p19-linker-p40 or p40-linker-p19.

56. The masked IL23 fusion protein of embodiment 54, wherein the single chain IL23 polypeptide is a p40-linker-p19 polypeptide that is fused to the second Fc polypeptide at the p40 polypeptide. 57. The masked IL23 fusion protein of embodiment 54, wherein the single chain IL23 polypeptide is a p19-linker-p40 polypeptide that is fused to the second Fc polypeptide at the p19 polypeptide. 58. The masked IL23 fusion protein of embodiment 56 or embodiment 57, wherein the single chain IL23 polypeptide is fused to the c-terminal end of the second Fc polypeptide. 59. The masked IL23 fusion protein of embodiment 56 or embodiment 57, wherein the single chain IL23 polypeptide is fused to the c-terminal end of the second Fc polypeptide and the masking moiety is fused to the c-terminal end of the first Fc polypeptide.

60. A recombinant polypeptide comprising a protease cleavable linker (PCL) wherein the protease cleavable linker comprises the amino acid sequence MSGRSANA (SEQ ID NO:10). 61. The recombinant polypeptide of embodiment 60 comprising two heterologous polypeptides, a first polypeptide located amino (N) terminally to the PCL and a second polypeptide located carboxyl (C) terminally to the PCL. 62. The recombinant polypeptide of embodiment 61 wherein the two heterologous polypeptides are selected from a cytokine polypeptide, an antibody, an antigen-binding fragment of an antibody and an Fc domain. 63. The recombinant polypeptide of embodiment 61 wherein the recombinant polypeptide comprises a cytokine polypeptide, a MM, and an Fc domain. 64. The recombinant polypeptide of embodiment 63 wherein the MM is a single-chain Fv (scFv) antibody fragment that binds to the cytokine or a cytokine receptor polypeptide or a cytokine-binding fragment thereof. 65. The recombinant polypeptide of embodiment 61 wherein the recombinant polypeptide comprises an antibody or antigen binding fragment thereof that binds a target, and a MM that binds to the antibody or antigen binding fragment thereof and blocks binding of the antibody or antigen binding fragment thereof to the target.

66. An isolated polypeptide comprising a PCL, wherein the PCL comprises the amino acid sequence of SEQ ID NO:10, wherein the PCL is a substrate for a protease, wherein the isolated polypeptide comprises at least one moiety (M) selected from the group consisting of a moiety that is located amino (N) terminally to the PCL (MN), a moiety that is located carboxyl (C) terminally to the PCL (MC), and combinations thereof, and wherein the MN or MC is selected from the group consisting of an antibody or antigen binding fragment thereof; a cytokine or a functional fragment thereof; a MM; a cytokine receptor or a functional fragment thereof; an immunomodulatory receptor, or functional fragment thereof; an immune checkpoint protein or a functional fragment thereof; a tumor associated antigen; a targeting domain; a therapeutic agent; an antineoplastic agent; a toxic agent; a drug; and a detectable label.

Unless defined otherwise, all technical and scientific terms used herein have the same meaning as is commonly understood by one of skill in the art to which the claimed subject matter belongs. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information. Terms understood by those in the art of antibody technology are each given the meaning acquired in the art, unless expressly defined differently herein.

It is to be understood that the general description and following detailed description are exemplary and explanatory only and are not restrictive of any subject matter claimed.

In this application, the use of the singular includes the plural unless specifically stated otherwise.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means±10% of the indicated range, value, sequence, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously. In addition, it should be understood that the individual single chain polypeptides or immunoglobulin constructs derived from various combinations of the structures and substituents described herein are disclosed by the present application to the same extent as if each single chain polypeptide or heterodimer were set forth individually. Thus, selection of particular components to form individual single chain polypeptides or heterodimers is within the scope of the present disclosure.

The section headings used herein are for organizational purposes only and are not to be construed as limiting the subject matter described.

It is to be understood that the methods and compositions described herein are not limited to the particular methodology, protocols, cell lines, constructs, and reagents described herein and as such may vary. It is also to be understood that the terminology used herein is for the purpose of describing particular embodiments only and is not intended to limit the scope of the methods and compositions described herein, which will be limited only by the appended claims.

### EXAMPLES

### Experimental Protocols

### Cloning

### Protocol 1: Cloning

The polypeptide sequences of clones presented in the following examples were reverse translated to DNA, codon optimized for mammalian cell expression, and gene synthesized. All sequences were preceded by an artificially designed signal peptide of sequence MRPTWAWWLFLVLLLALWAPARG (SEQ ID NO: 1) (Barash S et al., Biochem and Biophys Res. Comm. 2002; 294, 835-842). For all sequences, vector inserts consisting of a 5′-EcoR1 restriction site, the signal peptide described above, the codon-optimized DNA sequence corresponding to clones presented in the following examples, a TGA or TAA stop codon, and a BamH1 restriction site-3′, were ligated into pTT5 vectors to produce expression vectors (Durocher Y et al., Nucl. Acids Res. 2002; 30, No. 2 e9). The resulting expression vectors were sequenced to confirm correct reading frame and sequence of the coding DNA.

### Mammalian Cell Transient Transfection and Protein Expression

### Protocol 2: Expi293™ Expression

Expi293™ cells were cultured at 37° C. in Expi293™ expression medium (Thermo Fisher, Waltham, Mass.) on an orbital shaker rotating at 125 rpm in a humidified atmosphere of 8% CO2. Each 1 mL of cells at a density of 3×10⁶ cells/mL was transfected with a total of 1 μg DNA. Prior to transfection the DNA was diluted in 60 μL Opti-MEM™ I Reduced Serum Medium (Thermo Fisher, Waltham, Mass.). In a volume of 56.8 μL Opti-MEM™ I Reduced Serum Medium, 3.2 μL of ExpiFectamine™ 293 Reagent (Thermo Fisher, Waltham, Mass.) was diluted and, after incubation for five minutes, combined with the DNA transfection mix to a total volume of 120 μL. After 20 minutes the DNA-ExpiFectamine™ 293 Reagent mixture was added to the cell culture. After incubation at 37° C. for 16-18 hours, 6 μL of ExpiFectamine 293 Transfection Enhancer 1 (Thermo Fisher, Waltham, Mass.) and 60 μL of ExpiFectamine 293 Transfection Enhancer 2 (Thermo Fisher, Waltham, Mass.) was added to the culture. Cells were incubated for five to seven days and supernatants were analyzed by non-reducing SDS-PAGE.

### Protocol 3: ExpiCHO™ Expression

ExpiCHO™ cells were cultured at 37° C. in ExpiCHO™ expression medium (Thermo Fisher, Waltham, Mass.) on an orbital shaker rotating at 125 rpm in a humidified atmosphere of 8% CO2. Each 1 ml of cells at a density of ˜6×10⁶ cells/ml was transfected with a total of 0.8 μg DNA. Prior to transfection the DNA was diluted in 40 μL OptiPRO™ SFM (Thermo Fisher, Waltham, Mass.). In a volume of 36.8 μL OptiPRO™ SFM, 3.2 μL of ExpiFectamine™ CHO reagent (Thermo Fisher, Waltham, Mass.) was diluted and, after incubation for one to five minutes, combined with the DNA transfection mix to a total volume of 80 μL. After one to five minutes the DNA-ExpiFectamine™ CHO Reagent mixture was added to the cell culture. After incubation at 37° C. for 18-22 hours, 6 μL of ExpiCHO™ Enhancer and 240 μL of ExpiCHO™ Feed (Thermo Fisher, Waltham, Mass.) were added to each culture. Cells were incubated for seven days and supernatants were harvested for protein purification.

### Protocol 4: CHO-3E7 Expression

CHO-3E7 cells at a density of 1.7-2×10⁶ cells/ml were cultured at 37° C. in FreeStyle™ F17 medium (Thermo Fisher, Watham, Mass.) supplemented with 4 mM glutamine (GE Life Sciences, Marlborough, Mass.) and 0.1% Pluronic F-68 (Gibco, Life Technologies). Cells were transfected with 1 μg DNA per 1 mL of cells (DNA comprised of Variant expression vector DNA mixtures and GFP/AKT/stuffer DNA in a 1:1 w/w ratio) using PEI-max (Polyscience, Philadelphia, Pa.) at a DNA:PEI ratio of 1:4 (w/w). Twenty-four hours after the addition of the DNA-PEI mixture, 0.5 mM valproic acid (final concentration), 1% w/v Tryptone (final concentration), and 1×antibiotic/antimycotics (GE Life Sciences, Marlborough, Mass.) were added to the cells, which were then transferred to 32° C. and incubated for 7 days prior to harvesting.

### Protocol 5: HEK293-6E Expression

HEK293-6E cells at a density of 1.5-2.2×10⁶ cells/ml were cultured at 37° C. in FreeStyle™ F17 medium (GIBCO Cat #A13835-01) supplemented with G418 (Wisent bioproducts cat #400-130-IG), 4 mM glutamine, and 0.1% Pluronic F-68 (Gibco Cat #24040-032). Cells were transfected with 1 μg DNA per 1 mL of cells (DNA comprised of Variant expression vector DNA mixtures and GFP/AKT/stuffer DNA in a 1:1 w/w ratio) using PEI-max (Polyscience, Philadelphia, Pa.) at a DNA:PEI ratio of 1:2.5 (w/w). Twenty-four hours after the addition of the DNA-PEI mixture, 0.5 mM Valproic acid (final concentration) and 0.5% w/v Tryptone N1 (final concentration) were added to the cells, which were then transferred to 37° C. and incubated for 7 days prior to harvesting.

### Protein Purification

### Protocol 6: Protein-A Affinity Purification 1

Supernatants from transient transfections were applied to slurries containing 50% mAb Select SuRe™ resin (GE Healthcare, Chicago, Ill.) and incubated overnight at 2-8° C. on an orbital shaker at 150 rpm. The slurries were transferred into chromatography columns and flow-throughs were collected. The resins were then washed with 5 Bed Volumes (BV) of resin Equilibration buffer (PBS). To elute the targeted proteins, 5.5 BV of acidic Elution Buffer (100 mM sodium citrate buffer pH 3.5) was added to the columns and collected in fractions. Elution fractions were then neutralized by adding 10% (v/v) 1 M Tris pH 9 to reach a final pH of 6-7. The protein content of each elution fraction was determined by 280 nm absorbance measurement using a Nanodrop™ or with a relative colorimetric protein assay. The most concentrated fractions were pooled, which correspond to at least 80% of the total eluted protein.

### Protocol 7: Protein-A Affinity Purification 2

Purification of antibodies from clarified supernatants was performed using batch binding followed by the Amicon® Pro Purification System (Millipore-Sigma, cat #AC S503012). A 10 kDa MW membrane cutoff was used in the ultrafiltration portion of the device. A quantity of 200 μl of 50% (v/v) slurry of mAb Select SuRe Resin™ (GE Healthcare, cat #17543802) was added to clarified supernatant samples and the samples incubated in an orbital shaker overnight. The next day, the samples were centrifuged and most of the spent supernatant manually removed from each tube. The mAb Select SuRe™ resin was re-suspended in the remaining liquid and added to the Amicon® Pro Purification device. The Amicon Pro purification device was then centrifuged to remove remaining spent culture supernatant. Each sample was then washed with 1.5 mL (15 bed volumes of dPBS (HyClone —Ca, —Mg [GE Healthcare, cat #SH30028.02]) and the wash collected by centrifugation. 0.5 mL (5 bed volumes) of elution buffer (100 mM sodium citrate pH 3) was added to the Amicon® Pro Purification device and the unit centrifuged. The eluted proteins were collected and the pH adjusted by adding 10% (v/v) of 1 M HEPES base. Protein concentration was determined using absorbance at 280 nm with a Nanodrop 2000™ instrument (Thermo-Fisher Scientific, cat #ND-2000). Purified antibodies were sterile-filtered (0.2 μm) and stored at 2-8° C. in polypropylene tubes.

### Protocol 8: Size-Exclusion Chromatography (SEC) Purification

Samples were loaded onto a Superdex 200 Increase 10/300 column (#28-9909-44, GE Healthcare Life Sciences, Marlborough, Mass.) on an Akta pure 25 chromatography system (GE Healthcare Life Sciences, Marlborough, Mass.) in PBS with a flow rate of 0.8 mL/min. Fractions of eluted protein were collected based on A280 nm and their purity were analyzed by non-reducing CE-SDS with LabChip™ GXII Touch (Perkin Elmer, Waltham, Mass.). Protein containing fractions of high purity were pooled and protein in final pools was quantitated based on A280 nm (Nanodrop™) post SEC.

### Protein Analytics

### Protocol 9: Capillary Electrophoresis (CE) Using LabChip™

Following protein-A affinity purification, purity of samples was assessed by non-reducing and reducing LabChip™ CE-SDS. LabChip™ GXII Touch (Perkin Elmer, Waltham, Mass.) analysis was carried out according to Protein Express Assay User Guide (PerkinElmer, Waltham, Mass.), with the following modifications. Samples at a concentration range of 5-2000 ng/μ1 were added to separate wells in 96 well plates (#MSP9631, BioRad, Hercules, Calif.) along with 7 μl of HT Protein Express Sample Buffer (#CLS920003, Perkin Elmer) and denatured at 90° C. for 5 mins. The LabChip™ instrument was operated using the LabChip™ HT Protein Express Chip (Perkin Elmer #760528) with HT Protein Express 200 assay setting.

### Protocol 10: UPLC-SEC

The masked and unmasked cytokine fusion protein variants were assessed by UPLC-SEC to determine their percentage of high molecular weight species. UPLC-SEC was performed using a Waters Acquity BEH200 SEC column (2.5 mL, 4.6×150 mm, stainless steel, 1.7 μm particles) (Waters LTD, Mississauga, ON) set to 30° C. and mounted on an Agilent Technologies 1260 infinity II system with a PDA detector. Run times consisted of 7 min and a total volume per injection of 2.8 mL with a running buffer of either 150 mM Sodium Phosphate pH 6.95, DPBS+0.02% Tween 20, or 200 mM KPO4, 200 mM KCl, pH7, at 0.4 mL/min. Elution was monitored by UV absorbance in the range 210-500 nm, and chromatograms were extracted at 280 nm. Peak integration was performed using OpenLAB™ CDS ChemStation™ software.

### Protocol 11: Differential Scanning Calorimetry (DSC)

The thermal stability and Tm of variants was assessed by DSC. 950 μL of purified samples at concentrations between 0.24 and 1.9 mg/mL in PBS were used for DSC analysis with a Nano DSC (TA instruments, New Castle, Del.). At the start of each run, buffer blank injections were performed to stabilize the baseline. Each sample was scanned from 25 to 95° C. at a 60° C./hr rate, with 60 psi nitrogen pressure. The resulting thermograms were referenced and analyzed using NanoAnalyze software to determine melting temperature (Tm) as an indicator of thermal stability.

### Protein Binding Experiments

### Protocol 12: IL12 Binding Determination by Surface Plasmon Resonance (SPR)

Fusion protein variants were tested for their binding to recombinant IL12 and affinities (KD) were determined by Surface Plasmon Resonance (SPR). Experiments were carried out on a Biacore™ T200 instrument (GE LifeSciences) at 25° C. in PBS pH 7.4+0.05% (v/v) Tween 20 (PBS-T) running buffer. Variants were captured onto the anti-human Fc-specific polyclonal antibody surface, followed by the injection of five concentrations of recombinant IL12. The anti-human Fc surface was prepared on a CMS Series S sensor chip (GE LifeSciences) by standard amine coupling as described by the manufacturer (GE LifeSciences). Briefly, immediately after EDC/NHS activation, a 25 μg/mL solution of anti-human IgG Fc (Jackson Immuno Research) in 10 mM NaOAc, pH 4.5, was injected at a flow rate of 5 μL/min for 360 seconds. The remaining active groups were quenched by a 420 s injection of 1 M ethanolamine hydrochloride-NaOH pH 8.5 at 10 μL/min. Next, variants for analysis were indirectly captured onto the anti-Fc surface by injecting 5 μg/mL solutions at a flow rate of 10 μL/min for 30s. Using multi-cycle kinetics, five concentrations of a two-fold dilution series of recombinant IL12 (Peprotech) starting at 2.5 nM with a blank buffer control were sequentially injected at 50 μL/min for 180 s with a 1800 s dissociation phase, resulting in a set of sensorgrams with a buffer blank reference. The same sample titration was also performed on a reference cell with anti-human Fc immobilized and no variants captured. The anti-human Fc surfaces were regenerated to prepare for the next injection cycle by one pulse of 10 mM glycine/HCl, pH 1.5, for 60 s at 30 μL/min. Double-referenced sensorgrams were analyzed using Biacore™ T200 Evaluation Software v3.0 and fit to the 1:1 Langmuir binding model.

### Mass Spectrometry

### Protocol 13: LTQ-Orbitrap Intact Mass Spectrometry

LC/MS was performed on fusion protein variants having protease cleavable linkers to identify the locations of cleavage and apparent abundance of cleaved species. Samples were treated with 20 mM DTT at 56° C. for 30 minutes and then deglycosylated overnight at 37° C. with a mixture of PNGaseF, neuraminidase, β-galactosidase and N-acetylglucosaminidase, and subjected to intact mass LCMS analysis using an Agilent E1P1100 Capillary LC (Binary Pump, Autosampler) coupled to an LTQ-Orbitrap-XL mass spectrometer via an Ion-Max electrospray source. A 2.1×30 mm POROS R2 column was used to desalt and separate the proteins. The HPLC column was housed in a Sidewinder LC column heater and the mobile phase was heated pre-column in an Isotemp Oven. The oven and the column heater were both set to 82.5-90° C. The LC mobile phases were 0.1% formic acid (solvent A) and acetonitrile (solvent B). The mass spectrometer was tuned for high mass analysis with the HCD collision gas set to “off”, “detection delay” set to “low”, and the FTMS detector resolution set at “7500”. The “spray voltage” was set to 3.8 kV, the “sheath gas” flowrate and the “auxiliary gas” flowrate were set at 40 and 20, respectively. The liquid chromatograph was set at a flow rate of 3 mL/min. A post-column splitter directed 100 μL/min of flow to the MS electrospray. The flow was diverted from the electrospray source for the first 1.5 minutes of the LC run to avoid contaminating the electrospray source. After 3 minutes, the gradient ramped from 20% to 90% solvent B in 3 minutes (linear gradient). After the linear gradient, the system re-equilibrated at 20% solvent B for 3 minutes. The raw protein mass spectra were transformed into a MassLynx-compatible file format using Databridge then deconvoluted to a molecular weight profile using MaxEnt.

### Protocol 14: Synapt Q-TOF Intact Mass Spectrometry

LC/MS was performed on fusion protein variants having protease cleavable linkers to identify the locations of cleavage. Samples were deglycosylated overnight at 37° C. with PNGaseF, neuraminidase, β-galactosidase and N-acetylglucosaminidase, and subjected to intact mass LCMS analysis using an Agilent HP1100 Capillary LC (Binary Pump, Autosampler) coupled to a Synapt G2-Si quadrupole time-of-flight mass spectrometer via a high flow electrospray ion source. A 2.1×30 mm POROS R2 column was used to desalt and separate the proteins. The HPLC column was housed in a Sidewinder LC column heater and the mobile phase was heated pre-column in an Isotemp Oven. The oven and the column heater were both set to 82.5-90° C. The LC mobile phases were 0.1% formic acid (solvent A) and acetonitrile (solvent B). The mass spectrometer was tuned using Glul-fibrinopeptide b to ensure optimal sensitivity and resolution: a 500 fmol/μL solution flowing at 1 μl/min should yield a minimum signal of 1e6 for the doubly protonated molecular ion at a resolution of 20,000. The electrospray and cone voltages were set to 3 kV and 150 V, respectively. The trap collision energy and the transfer collision energy were both set at 4V. The desolvation gas flow was 600 L/min. The LockSpray option was turned off as this interfered with acquisition of the protein mass spectra. However, mass accuracy of the protein multiply charged ions did not deteriorate as a result. The liquid chromatograph was set at a flow rate of 3 mL/min. A post-column splitter directed 100 μL/min of flow to the MS electrospray. The flow was diverted from the electrospray source for the first 1.5 minutes of the LC run to avoid contaminating the electrospray source. After 3 minutes, the gradient ramped from 20% to 90% solvent B in 3 minutes (linear gradient). After the linear gradient, the system re-equilibrated at 20% solvent B for 3 minutes. The raw protein mass spectra were deconvoluted to generate molecular weight profiles using MaxEnt.

### Example 1: Design of Parental Non-Masked IL12 and IL23 HetFc Fusion Proteins

Non-masked parental IL12 fusion proteins to the Heterodimeric Fc (HetFc′) were designed using three different approaches:
A) The p40 subunit fused N- or C-terminally to one of the HetFc chains with a peptide linker, and the p35 subunit co-expressed
B) The p35 subunit fused N- or C-terminally to one of the HetFc chains with a peptide linker, and the p40 subunit co-expressed
C) The p35 subunit fused C-terminally to the p40 subunit by a peptide linker to create single-chain IL12 (′ scIL12′), and scIL12 fused C-terminally to one of the HetFc chains using a peptide linker

Specific non-masked parental IL12 HetFc fusion constructs are summarized in Table 1 and diagrammed in FIG. 1.

TABLE 1
Non-masked parental IL12 HetFc fusion protein variants

| Variant ID | HetFc 1 clone ID | HetFc 2 clone ID | Other clone ID |
|---|---|---|---|
| v22945 | CL_#17875^{a} | CL_#12153 | CL_#17871 |
| v22946 | CL_#17877 | CL_#12153 | CL_#17871 |
| v22948 | CL_#17879 | CL_#12153 | CL_#17872 |
| v22949 | CL_#17875 | CL_#17881 | CL_#17871 |
| v22951 | CL_#17876 | CL_#12153 | NA |
| v23086 | CL_#17942 | CL_#12153 | CL_#17872 |
| v23087 | CL_#17942 | CL_#17880 | CL_#17872 |

^{a}Structural summaries and SEQ IDs for all clones are given in Table 23

Non-masked parental IL23 fusion proteins to the HetFc were designed as described above for IL12 but with the p19 subunit used instead of the p35 subunit. Specific constructs are summarized in Table 2.

TABLE 2
Non-masked parental IL23 HetFc fusion protein variants

| Variant ID | HetFc 1 clone ID | HetFc 2 clone ID | Other clone ID |
|---|---|---|---|
| v23046 | CL_#17906 | CL_#12153 | CL_#17871 |
| v23048 | CL_#17907 | CL_#12153 | CL_#17871 |
| v23051 | CL_#17879 | CL_#12153 | CL_#17908 |
| v23088 | CL_#17942 | CL_#12153 | CL_#17908 |
| v23091 | CL_#17945 | CL_#12153 | NA |

### Example 2: Design, Selection and Characterization of Protease Cleavage Sites

The following example describes the design, identification and characterization of cleavage site(s) that are specifically cleaved by serine proteases or other tumour microenvironment specific proteases, such as urokinase plasminogen activator (uPA) and matriptase.

UPA and matriptase were identified as TME-specific proteases through literature and genome-wide mRNA analysis between healthy individuals and patients with various primary tumour or metastasis (Hoadley et al, Cell, 2018; GTEX Consortium, Nature, 2017; Robinson et al, Nature, 2017).

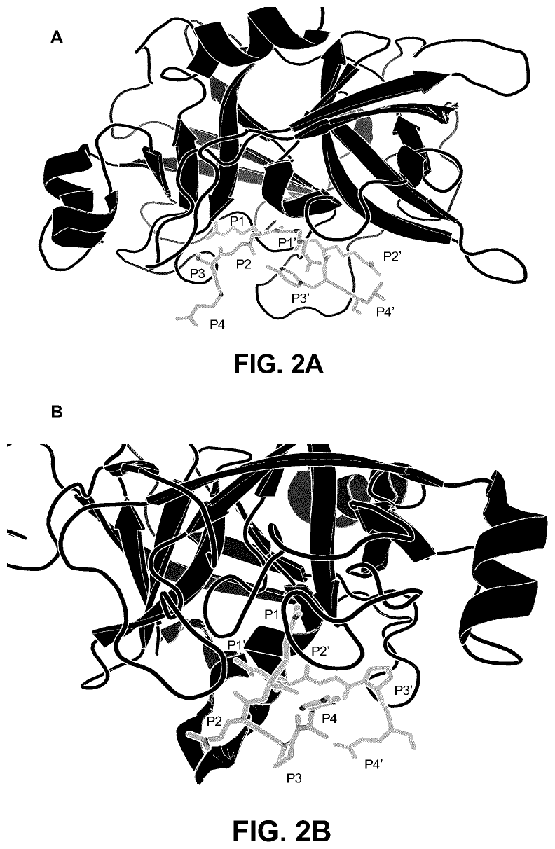

A library of cleavage sites that is specifically cleaved by TME-specific proteases was designed to release one or multiple cleavable moieties from a fusion protein (e.g., from a masked cytokine or antibody). Such masked molecules may include antibodies, antibody drug conjugates, antibody fusion protein, or other related molecules known in the art and described herein. The selection of an 8 amino acid residue long cleavage site (P4-P4′) is based on previous publications and structural observations indicating that residues within this range influence the specificity and catalytic activity of uPA and matriptase (FIGS. 2A and 2B).

TSGRSANP (SEQ ID NO: 2) and LSGRSDNH (SEQ ID NO: 3) have been identified as uPA and matriptase specific sequences, respectively, and are used as benchmarks for all activity assays. SGR(S>R,K,A,)X, where X represents a variety of amino acid residues, but was most often alanine, glycine, serine, valine, or arginine, has been identified as a consensus sequence for uPA (Ke et al., JBC, 1997, 272(33), 20456) and is used as a comparator.

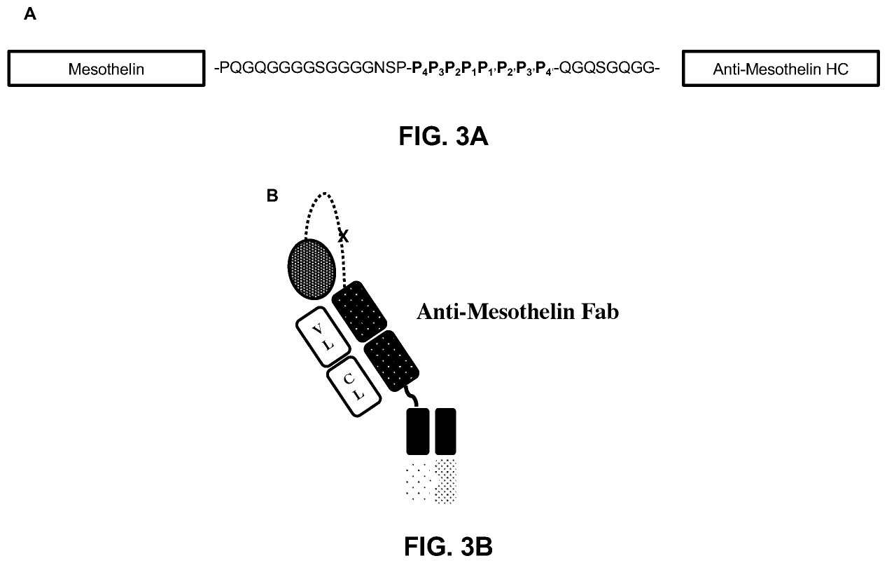

The library was designed and tested in a one-armed antibody format, where a cleavable moiety composed of a mesothelin (uniprot entry Q13421) fragment is linked by a flexible cleavable linker to the N-terminus of an anti-mesothelin Fab-Fc through the heavy chain (FIGS. 3A and 3B).

Design of uPa/Matriptase Cleavage Sites

Previous publications and art on peptide sequences that are cleaved by uPA were used to identify positions that can impact cleavage activity. However, cleavage in the context of a peptide or a protein will be significantly different in terms of kinetics (K_{cat}, Kₘ and Vₘₐₓ). Exposure of the site and flexibility/rigidity of the environment in a protein setting impacts the rate at which the site is cleaved because adopting the conformation required for the active conformation is likely less energetically favorable. Thus, transferability of a peptide with high specific activity to a larger therapeutic molecule cannot be easily predicted.

As a starting point, sequences known to be cleaved by uPA were selected from the literature (Ke et al., JBC, 1997, 272(33), 20456; Coomb et al, JBC, 1998, 273(8), 4323; Bergstrom et al, Biochemistry, 2003, 43, 5395). We then explored multiple amino acid substitutions at all positions from P4-P4′ through different strategies:

Strategy #1:
Alternate sequences from SGR consensus, that were known to be cleaved by uPA in peptide phage display libraries and met the following criteria:
no large hydrophobic residues such as Y, F, W or H at P4,
no Y, F or R residues at P3,
no cysteine in the sequence
and no R at P1′.
In instances where the cleavage site did not span the 8 residues, additional residues were added at the N-terminus and C-terminus to complete the motif.

Strategy #2:
Consensus sequence for uPA (SGRS) were combined with amino acids at positions P2′-P4′ that were known to induce uPA specificity (Ke et al., JBC, 1997, 272(33), 20456.). Based on crystal structures (FIGS. 2A and 2B) P3 and P4 are important for uPA and matriptase specificity, and thus P3 and P4 were individually modified for residues T, I, G, H, K, V and K, S, T, A, R, M, respectively.

Strategy #3:
Fragments of sequences that showed either high specificity or activity for uPA, in the literature and in our experimental data generated above were combined to generate sequences with improved properties. Properties evaluated include high specificity and activity for selected serine proteases such as uPA and matriptase.

The cleavage activity by uPA, matriptase and plasmin of 35 sequences, generated as discussed above, was evaluated in the context of a fusion protein in vitro under physiologically relevant conditions as described below. Some cleavage sequences performed comparably to the benchmark cleavage sequences for uPA, matriptase and plasmin cleavage. Some sequences showed no specific uPA cleavage and comparable or higher cleavage by matriptase and/or plasmin as compared to the benchmark. Other sequences showed no specific uPA cleavage and lower cleavage by matriptase and/or plasmin as compared to the benchmark. Representative results are reported in Table 3 (SEQ ID NOs: 2-10). A plasmin cleavage assay was used as a proxy for general serine protease resistance. Sample production is described in General Methods as Protocol 4 and Protocol 7.

### Enzymatic Digestions

For initial protease digestion screening, aliquots of purified antibodies were buffer exchanged into DPBS+0.01% [v/v] PS-20 using a PD MultiTrap G-25 desalting plate (GE

Healthcare cat #28-9180-06). All variants were digested with human uPA (Cedarlane cat #1310-SE-010), plasmin (Cedarlane cat #MD-14-0070P) or matriptase (Cedarlane cat #3946-SE-010) at a ratio of 1:50 (w/w). Digestion samples in a 96-well microtiter plates (BioRad Laboratories, cat #HSP9601) were incubated at room temperature (22° C.) for 48 h. Uncleaved variant controls incubated in parallel were included for each digestion experiment performed. Each digest or control sample was analyzed by non-reducing SDS-PAGE.

### Non-Reducing SDS-PAGE

Protein digests were analyzed by non-reducing SDS-PAGE using the NuPAGE XCell MiniCell (cat #EI001) or Midi Cell (cat #WR0100) with NuPAGE Bis-Tris gels (Life Technologies, Thermo-Fisher Scientific). Samples were prepared in LDS sample buffer (Life Technologies, Thermo-Fisher Scientific, cat #NP007) and heated at 70° C. for 10 min. Gels were stained using SYPRO Ruby protein gel stain (Life Technologies, Thermo Fisher Scientific, cat #S-12000).

TABLE 3
Cleavage of selected representative
sequences by specific and non-
specific serine proteases

| | | | Uroki- | | |
|---|---|---|---|---|---|
| | | | nase | | |
| | | | plas- | | |
| | Cleavage | | min- | | |
| | site | SEQ | ogen | | |
| | se- | ID | acti- | | Plas- |
| Variant* | quences | NO** | vator | Matriptase | min |
| 22775- | LSGRSDNH | 3 | +++ | ++++ | +++ |
| Benchmark | | | | | |
| (was CV1) | | | | | |
| 22776- | TSGRSANP | 2 | ++++ | ++++ | +++ |
| Benchmark | | | | | |
| (was CV2) | | | | | |
| 22780- | GSGRSAQV | 4 | ++ | ++ | ++++ |
| uPA | | | | | |
| consensus | | | | | |
| (was CV3) | | | | | |
| 22781 | GSSRNADV | 5 | − | ++ | ++++ |
| (was CV4) | | | | | |
| 22783 | GTARSDNV | 6 | − | +++ | ++++ |
| (was CV5) | | | | | |
| 22787 | GGGRVNNV | 7 | − | ++ | + |
| (was CV6) | | | | | |
| 22789 | MSARILQV | 8 | − | ++++ | ++++ |
| (was CV7 | | | | | |
| CV8 | GKGRSANA | 9 | − | ++ | ++++ |
| 22804 | MSGRSANA | 10 | ++++ | ++++ | ++ |
| (was CV9) | | | | | |
| 22793 | GTGRSANA | 346 | ++++ | ++++ | +++ |
| 22802 | ASGRSANA | 347 | +++ | ++ | +++ |
| 22777 | GSGKSANA | 348 | ++ | ++++ | ++++ |
| 22778 | GSGRNAQV | 349 | ++ | ++++ | ++++ |
| 22779 | GSGKNAQV | 350 | ++ | +++ | ++++ |
| 22782 | GTARLRGV | 351 | + | ++++ | ++++ |
| 22784 | GTSRMGTV | 352 | + | ++++ | ++++ |
| 22785 | GTSRQAQV | 353 | ++ | ++++ | ++++ |
| 22786 | AIKRSAQV | 354 | ++ | ++++ | ++++ |
| 22788 | STARMLQV | 355 | + | ++++ | ++++ |
| 22790 | GTQRSTGV | 356 | +++ | +++ | ++++ |
| 22791 | GTRRDRIV | 357 | ++ | ++++ | ++++ |
| 22792 | GVARNYKV | 358 | − | ++++ | +++ |
| 22794 | GGGRSANA | 359 | ++ | ++++ | +++ |
| 22795 | GVGRSANA | 360 | +++ | +++ | ++++ |
| 22796 | GIGRSANA | 361 | ++ | ++++ | +++ |
| 22797 | GHGRSANA | 452 | ++ | ++++ | +++ |
| CV27 | KSGRSANA | 453 | − | ++++ | +++ |
| CV28 | TSGRSANA | | +++ | +++ | ++++ |
| 22801 | SSGRSANA | 365 | +++ | +++ | +++ |
| 22803 | RSGRSANA | 366 | +++ | +++ | ++ |

Each variant contains an antigen ECD fragment fused through a linker containing the indicated cleavable sequence to an anti-domain antibody heavy chain containing HetFc1 mutations, with a domain structure of: antigen ECD fragment-PQGQGGGGSGGGGNSP-Cleavable Sequence-QGQSGQGG-Anti-domainvH-CH1-HetFc. Each variant also includes the Clone_#12155 HetFc2 and anti-domain antibody light chain.
**Cleavage sequence SEQ ID NO. For Clone domain structure see Table 23.
++++: >90% cleavage observed; +++: 75% cleavage observed; ++: 50% cleavage observed;
+: <25% cleavage observed;
−: no specific cleavage observed
The consensus cleavage site of uPa is highlighted in bold.

Cleavage sites in variants v22781, v22783 and v22789, which were shown to be cleaved by uPA in a peptide phage display library, were not transferable to an antibody fusion protein. These results highlight the impact of the surrounding environment, in terms of flexibility, site accessibility and local structure, on the cleavage site's activity.

The cleavability of the designed cleavage sequences by matriptase and plasmin has not been reported previously and spans a range of cleavability based on the different sequences.

Suitable cleavage sequences were selected based on positive and negative selection of the sites with different proteases. All sequences were clustered in the following categories, where cleavage by plasmin was used as a proxy for protease resistance:

1) elevated protease resistance (<25% cleaved by plasmin)

2) efficient matriptase cleavage activity only (>90% cleaved by matriptase)

3) efficient uPA and matriptase activity with high protease resistance (>90% cleaved by matriptase, <50% cleaved by plasmin)

4) efficient uPA and matriptase activity with moderate protease resistance (>90% cleaved by matriptase and uPa, <75% cleaved by plasmin)

5) intermediate activity for uPA and matriptase with moderate protease resistance (>50% cleaved by matriptase, <75% cleaved by plasmin)

6) intermediate activity for uPA and matriptase with low protease resistance (>50% cleaved by matriptase and uPa, ˜90% cleaved by plasmin)

Representative sequences with diverse properties were further characterized through enzymatic assays with uPA and matriptase under different conditions to mimic possible tumour microenvironment conditions. As the tumour microenvironment is often subjected to hypoxia as well as various resistance mechanisms that promote tumour growth and induce a lower local pH (Tannock and Rotin, 1989, Cancer Research, 49, 4373), representative sequences were assessed for their cleavage activity at different pH conditions ranging from pH 6.0 to 7.4.

The cleavage activity by uPA and matriptase of 7 representative sequences with diverse properties, as discussed above, was evaluated in the context of a fusion protein at pH 6.0 and 7.4. Some cleavage sequences were identified that performed comparably to the benchmark for uPA and matriptase at both pH. Some sequences showed comparable specific matriptase cleavage and higher cleavage by uPA as compared to the benchmark. Other sequences showed no specific uPA cleavage and lower or comparable cleavage by matriptase at both pH as compared to the benchmark. Representative results are reported in Table 4.

### Enzymatic Digestions

Samples were incubated at either pH 6 (buffer exchanged in DPBS+0.01% [v/v] PS-20 pH adjusted with HCl using Zebaspin 754, desalting columns (Thermo-Fisher Scientific, cat #89877)) or pH 7.4 (DPBS+0.01% [v/v] PS-20) in digests containing either matriptase (Cedarlane, cat #3946-SE-010) or uPa (Cedarlane, cat #1310-SE-010) at a ratio of 1:50 (w/w) in capped vials with inserts to minimize sample evaporation (Chromatographic Specialties Inc., cat #CQ2026). Samples were incubated at 37° C. for 48h. Control samples containing variant and buffer without enzyme were incubated in parallel for 48 h. All samples were analyzed by non-reducing SDS-PAGE as described above.

TABLE 4
Cleavage level of representative sequences by uPA and matriptase
at pH ranges representative of the tumour environment

| | uPa | uPa | Matriptase | Matriptase |
|---|---|---|---|---|
| | Cleavage | Cleavage | Cleavage | Cleavage |
| Variant | pH 6.0 | pH 7.4 | pH 6.0 | pH 7.4 |
| v22776 - | ++ | ++++ | ++++ | ++++ |
| Benchmark | | | | |
| v22780 - | ++ | ++++ | ++++ | ++++ |
| uPa Consensus | | | | |
| v22787 | − | − | + | ++ |
| v22789 | − | − | +++ | ++++ |
| v22793 | ++++ | ++++ | ++++ | ++++ |
| v22802 | ++ | ++++ | ++++ | ++++ |
| v22804 | ++ | ++++ | ++++ | ++++ |

++++: >90% cleavage observed;
+++: 75% cleavage observed;
++: 50% cleavage observed;
+: <25% cleavage observed;
−: no specific cleavage observed.

The sequences tested have different pH dependence for uPA and matriptase. All sequences had reduced uPA activity at low pH, but v22804 retained similar activity levels to the benchmark. Matriptase cleavage was also reduced at lower pHs for most variants. V22804 performed equally to the benchmark and consensus sequences in this assay as the samples were readily cleaved within 48h.

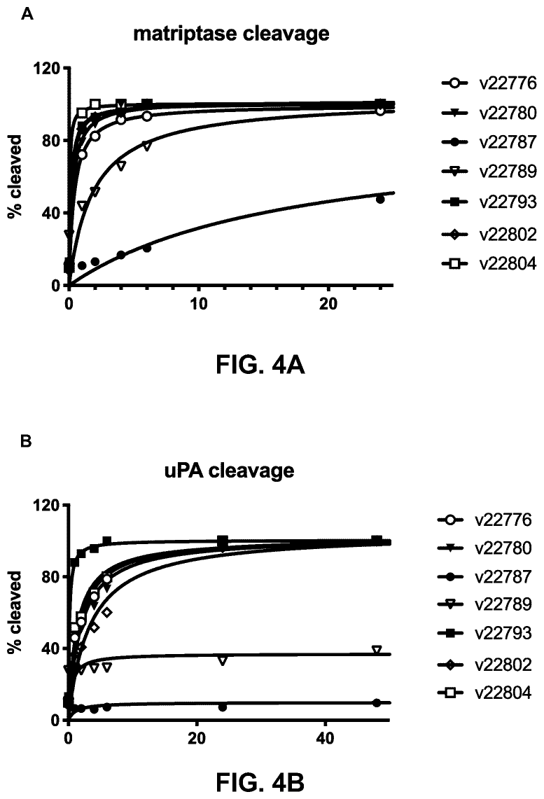

The cleavage activity by uPA and matriptase of 7 sequences identified above was further characterized in the context of a fusion protein in vitro under physiologically relevant conditions. We identified cleavage sequences that performed comparably or better than the benchmark cleavage sequences for uPA and matriptase cleavage. Other sequences showed no specific uPA cleavage and lower cleavage by matriptase as compared to the benchmark. Representative results are reported in FIGS. 4A and 4B.

### Kinetic Studies

For kinetic cleavage studies of selected antibodies, samples were buffer exchanged into DPBS+0.01% [v/v] PS-20 using 0.5 mL Zebaspin desalting columns (Thermofisher Cat #89882). Digests using either human matriptase (Cedarlane, cat #3946-SE-010) or uPA (Cedarlane, cat #1310-SE-010), with either enzyme at a ratio of 1:50 (w/w). Digestion reactions were setup in capped vials with inserts to minimize sample evaporation (Chromatographic Specialties Inc., cat #CQ2026) and incubated at 37° C. for 1 h, 2h, 4h, 6h, 24 h, 48h or 5 days. Antibody samples incubated under the same conditions without added enzyme served as controls. Samples for controls without enzyme and digests including enzyme for each time point were analyzed by non-reducing SDS-PAGE as described above.

The cleavage site within v22804 (MSGRSANA; SEQ ID NO:10) was identified as a suitable lead cleavage sequence that has high specific cleavage activity for uPA and matriptase, while being resistant to other serine protease such as plasmin. Variant v22804 showed high specific cleavage activity by uPA and matriptase and has comparable or improved properties compared to the consensus and benchmark sequences (Table 3, Table 4 and FIGS. 4A-4B).

### Example 3: Preparation of Anti-IL12/23 ScFv Masks

This example describes the re-formatting of anti-IL12/23 antibodies into single-chain variable fragment(s), scFv(s), to be used as masking moieties when fused to IL12/23 HetFc fusion proteins.

To create masked IL12 HetFc fusion proteins where the activity of IL12 is reduced compared to the parental non-masked IL12 HetFc fusion proteins described in Example 1, a polypeptide domain with affinity for IL12 that reduces IL12 binding to either or both of its receptors can be attached to the parental IL12 HetFc fusion proteins through protease-cleavable linkers. The polypeptide can be an antibody, specifically a Fab or scFv with affinity for IL12. Existing binders for IL12 are for example the antibodies Briakinumab and Ustekinumab.

Fusing an scFv mask instead of a Fab mask to parental IL12 HetFc fusion proteins may be superior because shorter linker lengths could be applied and the light chain would not need to be co-expressed. In addition, an scFv mask fusion would be compatible with the addition of Fab targeting arms to the masked IL12 HetFc, whereas a Fab mask would require that additional engineering be employed to prevent incorrect pairing between the heavy and light chains of the masking and targeting Fabs.

ScFv constructs of Briakinumab (Table 5) were created in two different orientations, with either the VH fused to the N-terminus of the VL by a (G4S)3 linker, or the VL fused to the N-terminus of the VH by a (G4S)3 linker. ScFv-HetFc fusions were then designed by fusing either scFv to the N- or C-terminus of one of the two HetFc heavy chains. A control Fab-HetFc fusion was constructed by fusing the Briakinumab VH-CH1 domains to one of the two HetFc chains and co-expressing the light chain VL-CL. Specific constructs are summarized in Table 6. To compare if the Briakinumab scFvs maintained affinity for IL12 compared to the Briakinumab Fab, scFv-HetFc and Fab-HetFc proteins were produced and tested for binding to recombinant IL12 by SPR.

TABLE 5
Briakinumab variable domain sequences

| | | SEQ |
|---|---|---|
| | | ID |
| Name | Sequence | NO: |
| VH | QVQLVESGGGVVQPGRSL | 11 |
| | RLSCAASGFTFSSYGMHW | |
| | VRQAPGKGLEWVAFIRYD | |
| | GSNKYYADSVKGRFTISR | |
| | DNSKNTLYLQMNSLRAED | |
| | TAVYYCKTHGSHDNWGQG | |
| | TMVTVSS | |
| VL | QSVLTQPPSVSGAPGQRV | 12 |
| | TISCSGSRSNIGSNTVKW | |
| | YQQLPGTAPKLLIYYNDQ | |
| | RPSGVPDRFSGSKSGTSA | |
| | SLAITGLQAEDEADYYCQ | |
| | SYDRYTHPALLFGTGTKV | |
| | TVL | |
| VHCDR1 | SYGMH | 13 |
| VHCDR2 | FIRYDGSNKYYADSVKG | 14 |
| VHCDR3 | HGSHDN | 15 |
| VLCDR1 | SGSRSNIGSNTVK | 16 |
| VLCDR2 | YNDQRPS | 17 |
| VLCDR3 | QSYDRYTHPALL | 18 |

TABLE 6
Briakinumab scFv-HetFc and Fab-HetFc variants

| Variant ID | HetFc 1 clone ID | HetFc 2 clone ID | Other clone ID |
|---|---|---|---|
| v23976 | CL_#18939 | CL_#12155 | CL_#18940 |
| v23977 | CL_#18942 | CL_#12155 | NA |
| v23978 | CL_#18943 | CL_#12155 | NA |
| v31807 | CL_#21417 | CL_#12155 | NA |
| v31854 | CL_#23360 | CL_#12155 | NA |
| v31855 | CL_#23361 | CL_#12155 | NA |
| v31857 | CL_#23363 | CL_#12155 | NA |

Variants were expressed in ExpiCHO™ or CHO-3E7 cells as described in Protocol 3 and Protocol 4. Initially, small-scale expression tests were performed using multiple Variant expression vector DNA mixtures with different molar ratios of the comprising Variant expression vectors. This was performed to account for differences in expression efficiency of the multiple expression vectors so that production of the complete Variant is maximized and production of incomplete variant or incorrectly formed species is minimized. Optimal molar ratios of Variant expression vector DNA were determined by visually assessing SDS-PAGE of culture supernatants for bands corresponding to the desired and undesired species.

Clarified supernatants from expression samples using optimal Variant expression vector DNA ratios were purified by protein-A affinity purification as described in Protocol 6. Following protein-A affinity purification, purity of samples was assessed by non-reducing and reducing LabChip™ CE-SDS as described in Protocol 9. Samples were further purified by SEC as described in Protocol 8.

Variants were tested for their binding to recombinant IL12 and affinities (KD) were determined by Surface Plasmon Resonance (SPR) as described in Protocol 12.

SPR results showed that the VL-(G4S)3-VH and VH-(G4)3-VL scFv-HetFc variants of Briakinumab bound recombinant IL12 with 1.8× and 3.1× higher affinity than the control Fab-HetFc v23976, respectively (Table 7). Furthermore, the affinity of the scFv for IL12 was not affected by more than 2.4×compared to the control Fab-HetFc v23976 by: a) fusion to the HetFc C-terminus via a peptide linker and protease cleavable sequence as in v31807 rather than to the N-terminus via a modified Fc hinge; b) the use of a longer GGS-(G3S)4-G linker as in v31854; c) addition of a disulfide bond (VH G44C; VL T100C) as in v31855; d) or addition of a protease cleavable linker between the VH and VL domains such as in v31857 (Table 7).

TABLE 7
SPR binding to recombinant IL12.

| Variant ID | ka (1/Ms) | kd (1/s) | KD (M) |
|---|---|---|---|
| v23976 | 3.50E+06 | 8.12E−05 | 2.32E−11 |
| v23977 | 3.88E+06 | 2.86E−05 | 7.38E−12 |
| v23978 | 2.17E+06 | 2.75E−05 | 1.27E−11 |
| v31807 | 1.85E+06 | 6.81E−05 | 3.68E−11 |
| v31854 | 1.69E+06 | 7.22E−05 | 4.30E−11 |
| v31855 | 2.48E+06 | 1.38E−04 | 5.59E−11 |
| v31857 | 3.25E+06 | 6.84E−05 | 2.11E−11 |

### Example 4: Briakinumab Mutants with Modified Affinity for IL12

Antibody-masked IL12 Fc fusion proteins may require scFvs with higher or lower affinity for IL12 depending on the desired potency reduction of the masked molecule and recovery of activity after proteolytic cleavage. To modulate the affinity of Briakinumab, we introduced single- and double-point mutations into the CDRs. CDR mutations were rationally designed by visual and ZymeCAD™ analyses of the crystal structure of Briakinumab Fab in complex with IL23 (Bloch et al. 2018, Immunity 48, 45-58; Protein Data Bank entry 5NJD). Mutations according to Kabat numbering for Briakinumab are listed in Table 8.

TABLE 8
Briakinumab scFv-HetFc modified affinity variants

| Variant ID | Mutation (Kabat) | HetFc 1 clone ID | HetFc 2 clone ID |
|---|---|---|---|
| v23977 | NA | CL_#18942 | CL_#12155 |
| v30684 | H_Y32A | CL_#22203 | CL_#12155 |
| v30686 | H_F27V | CL_#22206 | CL_#12155 |
| v30687 | H_Y52AV | CL_#22207 | CL_#12155 |
| v30688 | H_R52E | CL_#22208 | CL_#12155 |
| v30689 | H_R52E_Y52AV | CL_#22209 | CL_#12155 |
| v30690 | H_H95D | CL_#22211 | CL_#12155 |
| v30691 | H_G96T | CL_#22212 | CL_#12155 |
| v30693 | H_H98A | CL_#22214 | CL_#12155 |

Methods

Variants were designed in the scFv-HetFc format, expressed in ExpiCHO™ and purified as described in Example 3. The affinity of variants for recombinant IL12 was determined by SPR as described in Example 3. The thermal stability of variants was assessed by DSC as described in Protocol 11.

Results

Variants showed a range of affinities (KD) for IL12 that were reduced by ˜8.5 to 145.8×compared to the control scFv-HetFc v23977 (Table 9). While association rates were increased somewhat by up to ˜2.6×, the dissociation rates (k-off) were increased by as much as ˜267.9×, leading to decreased KDs overall.

The thermal stability of the mutated variants was maintained, with no more than a 0.7° C. reductions in Tm compared to WT control variant v23977. Variants containing the H_R52E mutation showed increased stability by 2-3° C. compared to v23977.

TABLE 9
Binding kinetics and thermal stability of Briakinumab
scFv-HetFc modified affinity variants

| Variant ID | ka (1/Ms) | kd (1/s) | KD (M) | Tm (° C.) |
|---|---|---|---|---|
| v23977 | 4.98E+06 | 6.27E−05 | 1.20E−11 | 63.7 |
| v30684 | 9.56E+06 | 1.68E−02 | 1.75E−09 | 63.0 |
| v30686 | 8.48E+06 | 2.12E−03 | 2.15E−10 | 63.5 |
| v30687 | 1.04E+07 | 2.01E−03 | 1.88E−10 | 63.2 |
| v30688 | 5.37E+06 | 7.78E−04 | 1.40E−10 | 66.7 |
| v30689 | 9.14E+06 | 8.36E−03 | 9.25E−10 | 65.7 |
| v30690 | 6.82E+06 | 3.77E−03 | 6.00E−10 | 65.1 |
| v30691 | 1.28E+07 | 6.14E−03 | 4.66E−10 | 65.8 |
| v30693 | 7.90E+06 | 8.14E−04 | 1.02E−10 | 63.1 |

### Example 5: Design of Antibody-Masked IL12 HetFc Fusion Proteins

The Briakinumab scFvs described in Examples 3 and 4 were used as masks and combined with the parental non-masked IL12 HetFc fusion proteins described in Example 1 to design antibody-masked IL12 HetFc fusion proteins.

Briefly, an scFv in either the VH-VL or VL-VH orientation was fused via a peptide linker to an available terminus of a parental non-masked IL12 HetFc fusion protein. A protease cleavage sequence as identified in Example 2 was incorporated into the linker between the IL12 HetFc fusion protein and the mask so that the mask would be released by protease cleavage, or between the masked IL12 HetFc fusion protein and IL12 so that the IL12 moiety would be released by protease cleavage. In some cases, an additional protease cleavage sequence was incorporated into the linker between the VH and VL domains of the scFv, which may aid in recovery of IL12 activity by destabilizing the scFv upon protease cleavage and accelerating its release. Linker lengths were determined by measuring distances between potential N- and C-terminal fusion sites in the crystal structure of the Briakinumab/IL23 complex (PDB code 5NJD, Bloch et al. (2018) Immunity 48: 45-58). Specific constructs are summarized in Table 10 and diagrammed in FIG. 5 to FIG. 9 and FIG. 32.

Because Briakinumab binds to the shared p40 subunit of IL12 and IL23, it is understood that antibody-masked IL23 constructs with the same architectures as variants described in Table 10 could be created by replacing the IL12 p35 subunit with the IL23 p19 subunit.

TABLE 10
Briakinumab scFv antibody-masked IL12 HetFc fusion proteins

| | | | |
|---|---|---|---|
| Variant ID | HetFc 1 clone ID | HetFc 2 clone ID | Other clone ID |

Briakinumab-masked IL12 HetFc fusion
proteins derived from parental v22946

| | | | |
|---|---|---|---|
| v29278 | CL_#21451 | CL_#17877 | CL_#17871 |
| v29240 | CL_#17877 | CL_#12153 | CL_#21415 |
| v29259 | CL_#17877 | CL_#12153 | CL_#21446 |
| v29279 | CL_#21452 | CL_#12153 | CL_#17871 |

Briakinumab-masked IL12 HetFc fusion
proteins derived from parental v22948

| | | | |
|---|---|---|---|
| v29277 | CL_#21451 | CL_#17879 | CL_#17872 |
| v29235 | CL_#17879 | CL_#12153 | CL_#21419 |
| v29258 | CL_#17879 | CL_#12153 | CL_#21447 |
| v29234 | CL_#21418 | CL_#12153 | CL_#17872 |

Briakinumab-masked IL12 HetFc fusion
proteins derived from parental v22945

| | | | |
|---|---|---|---|
| v29231 | CL_#17875 | CL_#12153 | CL_#21415 |
| v29232 | CL_#21416 | CL_#12153 | CL_#17871 |
| v29233 | CL_#21417 | CL_#17875 | CL_#17871 |
| v29257 | CL_#17875 | CL_#12153 | CL_#21446 |

Briakinumab-masked IL12 HetFc fusion
proteins derived from parental v23086

| | | | |
|---|---|---|---|
| v29237 | CL_#21417 | CL_#17942 | CL_#17872 |
| v29238 | CL_#21421 | CL_#12153 | CL_#17872 |
| v29239 | CL_#17942 | CL_#12153 | CL_#21419 |

Briakinumab-masked IL12 HetFc fusion
proteins derived from parental v22951

| | | | |
|---|---|---|---|
| v29243 | CL_#21423 | CL_#12153 | NA |
| v29244 | CL_#21417 | CL_#17876 | NA |
| v31277 | CL_#22735 | CL_#22279 | NA |
| v32041 | CL_#23512 | CL_#22279 | NA |
| v32299^{a} | CL_#23364 | CL_#22279 | NA |
| v32453 | CL_#23512 | CL_#23710 | NA |
| v32862^{b} | CL_#24224 | CL_#22279 | NA |
| v35426^{c} | CL_#26498 | CL_#22279 | NA |
| v35436^{d} | CL_#26503 | CL_#22279 | NA |

^{a}derived from v31277 (see FIGS. 2A-2B) but containing the H_Y32A mutation to reduce mask affinity.
^{b}derived from v31277 (see FIGS. 2A-2B) but with an alternate non-cleavable linker between the scFv VH and VL domains.
^{c}derived from v31277 (see FIGS. 2A-2B) but with an alternate non-cleavable linker between the scFv VH and VL domains, and the H_F27V mutation to reduce mask affinity.
^{d}derived from v32862 but with an alternate non-cleavable linker between the HetFc and scFv VH domains.

### Example 6: Design of Receptor-Masked IL12 HetFc Fusion Proteins

In addition to antibodies that bind IL12 as described in Example 3, fragments of the cognate IL12 receptors, IL12Rβ1 or IL12Rβ2, can be used as masking moieties when fused to parental non-masked IL12 HetFc fusion proteins. Receptor-masked IL12 HetFc fusion proteins were designed by linking a polypeptide chain of a portion of the ECD of human IL12Rβ2 to the parental non-masked IL12 HetFc fusion proteins described in Example 1, with a protease cleavage sequence as identified in Example 2 incorporated into either the linker between the IL12 HetFc fusion protein and the mask so that the mask is released by protease cleavage, or between the masked IL12 HetFc fusion protein and IL12 so that the IL12 moiety is released by protease cleavage. Specific constructs are summarized in Table 11 and diagrammed in FIG. 5 to FIG. 9.

It is understood that receptor-masked IL23 variants with the same architectures as variants described in Table 11 could be created by replacing the IL12 p35 subunit with the IL23 p19 subunit and replacing the portion of the IL12Rβ2 ECD used as a mask with a corresponding portion of the IL23R ECD.

TABLE 11
IL12Rβ2 receptor-masked IL12 HetFc fusion proteins:

| | | | |
|---|---|---|---|
| Variant ID | HetFc 1 clone ID | HetFc 2 clone ID | Other clone ID |

IL12Rβ2-masked IL12 HetFc fusion
proteins derived from parental v22951

| | | | |
|---|---|---|---|
| v24013 | CL_#18953 | CL_#17876 | NA |
| v24019 | CL_#12153 | CL_#18957 | NA |
| v32044 | CL_#23513 | CL_#22279 | NA |
| v32045* | CL_#22672 | CL_#22279 | NA |
| v32455 | CL_#23513 | CL_#23710 | NA |

IL12Rβ2-masked IL12 HetFc fusion
proteins derived from parental v23086

| | | | |
|---|---|---|---|
| v24014 | CL_#18953 | CL_#17942 | CL_#17872 |

IL12Rβ2-masked IL12 HetFc fusion
proteins derived from parental v22945

| | | | |
|---|---|---|---|
| v24015 | CL_#18953 | CL_#17875 | CL_#17871 |

IL12Rβ2-masked IL12 HetFc fusion
proteins derived from parental v22948

| | | | |
|---|---|---|---|
| v24016 | CL_#18954 | CL_#17879 | CL_#17872 |

IL12Rβ2-masked IL12 HetFc fusion
proteins derived from parental v22946

| | | | |
|---|---|---|---|
| v24017 | CL_#18954 | CL_#17877 | CL_#17871 |
| v24018 | CL_#18956 | CL_#12153 | CL_#17871 |

*Identical to v24013 but with the N-terminal R of p35 removed. In order to prevent cleavage between the Gly-Ser linker and the p35 N-terminus, the N-terminal arginine of p35 was removed such that the p35 sequence started with Asn2 (see also Example 8).

### Example 7: Production and Characterization of IL12 HetFc Fusion Proteins

This Example describes the expression and purification of parental and masked IL12 HetFc fusion proteins, and their characterization for monodispersity by UPLC-SEC.

Methods

Small-scale expression tests were performed in Expi293™, CHO-3E7, or HEK293-6E cells as described in Example 3 using multiple Variant expression vector DNA mixtures with different molar ratios of the comprising Variant expression vectors. Optimized molar ratios of Variant expression vector DNA for each Variant were then used for larger Expi293™, CHO-3E7, or HEK-293 expressions as described in Protocols 2, 4, and 5, and proteins were purified by pA and SEC as described in Protocols 6 and 8. UPLC-SEC post pA and post SEC was performed as described in Protocol 10.

Results

Yields post protein-A purification per L of transfection culture were in the range of 141-248 mg for parental IL12 HetFc fusion proteins, 72-182 mg for receptor-masked IL12 HetFc fusion protein variants and ˜70-418 mg for antibody-masked IL12 HetFc fusion variants. Exceptions were parental variant v23087 and masked variants v24016 and v24019, which had little to no visible protein expression by SDS-PAGE at small scale and were not scaled-up, and masked variants v32862 and v35426, which were not expressed in this group. UPLC-SEC analysis of protein-A purified material showed that variants where IL12 is fused to the N-terminus of the Fc (derived from parental variants v22946 and v22948) generally showed higher levels of high molecular weight species compared to variants where IL12 was fused to the C-terminus of the Fc (derived from parental variants v22945, v23086, and v22951). The UPLC-SEC profile of v29258 was very heterogeneous and this variant was not SEC purified. After SEC purification, variants displayed >85% monodispersity by UPLC-SEC, except for parental variant v22949, which was recovered with poor yield from SEC purification and showed ˜53% monodispersity by UPLC-SEC. Due to their poor expression or biophysical behavior, parental variants v23087 and v22949 were not used to design masked variants.

Antibody-masked variants that possess a second protease cleavage site incorporated between the scFv VH and VL domains, e.g. v31277 and v32299, displayed additional bands in reducing LabChip™ CE-SDS analysis that correspond to cleavage between the VH and VL. This pre-cleavage was observed in samples expressed from CHO cultures but not from HEK cultures, and corresponded to between 1.6 and 7.5% of the total HetFc-mask protein chain. One sample of v31277 that displayed 3.9% pre-cleavage by reducing LabChip™ CE-SDS analysis was also assessed by intact LC-MS according to Protocol 13 and displayed a 6% apparent abundance of the pre-cleaved species, and the location of pre-cleavage was confirmed to be within the matriptase cleavage motif between the scFv VH and VL.

### Example 8: Matriptase Cleavage of IL12 HetFc Fusion Proteins

To test if protease treatment would effectively cleave at the designed cleavage sequences within the masked IL12 HetFc fusion proteins of various geometries, the masked variants were digested with matriptase. Cleavage was assessed by LabChip™ CE-SDS analysis. Parental non-masked variants were also digested with matriptase to assess whether any non-specific cleavage events occur in IL12 or the HetFc.

Methods

Masked IL12 HetFc fusion proteins were incubated for 24 hours with matriptase (R&D Systems) at a molar ratio of 1:50 (matriptase:Protein) in a total reaction volume of 25 μL PBS-T pH 7.4 at 37° C. Non-reducing and reducing LabChip™ CE-SDS analysis was carried out to assess the degree of digestion, and LC/MS was performed as described in Protocol 14 to identify the locations of cleavage.

Results

Complete cleavage was observed by reducing LabChip™ CE-SDS analysis for all variants tested, as assessed by the disappearance of bands corresponding to the full-sized protein chains containing designed cleavage sequences compared to the same variant without matriptase digestion, and the appearance of bands corresponding approximately in MW to the expected species post-cleavage. Cleavage of IL12 outside of designed protease cleavage sequences was also observed by CE-SDS, and the cleavage sites were determined by LCMS. IL12 was cleaved within the p40 domain in a loop of sequence . . . QGKSK/REKK . . . (SEQ ID NO:19; residues 256-264 of SEQ ID NO:22) (cleavage location indicated by “/”) also known as the heparin-binding loop (Hasan et al. J Immunology 1999; 162: 1064-1070), and at the N-terminus of the p35 domain in variants where p35 was fused with a glycine-serine type linker to the HetFc or the p40 subunit, such as in v22951 ( . . . GGSR/NLPV . . . ) (see clone 17876 as set forth in SEQ ID NO:25).

### Example 9: Effects of IL12 HetFc Fusion Proteins+/−Matriptase on NK Cell Relative Abundance In Vitro

To determine the cytokine activity of masked and non-masked IL12 HetFc fusion proteins, NK cells were stimulated with purified variants, with or without matriptase pre-treatment, and relative cell abundance was measured as described below.

Methods

NK cell culture: Minimum Essential Medium alpha (ThermoFisher, Waltham, Mass.) supplemented with 0.1 mM 2-mercaptoethanol (ThermoFisher, Waltham, Mass.), 100 U/mL recombinant IL2 (Peprotech, Rocky Hill, N.J.), 12.5% human AB off-the-clot serum (Zen-Bio Inc., Research Triangle Park, N.C.), and 12.5% fetal bovine serum (ThermoFisher, Waltham, Mass.). Cells were maintained in vertical T75 flasks (VWR, Radnor, Pa.) an incubator at 37° C. and 5% carbon dioxide. The cells were replenished with fresh media with IL2 every 3 days.

Sample preparation: One day prior to the assay, two aliquots of each variant sample were thawed from −80° C. storage. Recombinant human matriptase was added to a single aliquot of each sample (R&D Systems, Minneapolis, Minn.) at a 50:1 sample to enzyme ratio, vortexed to mix, and incubated overnight at 37° C. for cleavage as described in Example 8.

NK Cell Assay: NK cells were cultured as above in growth medium without IL2 (assay media) for 12 hours, harvested in a 50 mL falcon tube and spun down at 400×G for 3 minutes to pellet cells. Cells were resuspended in assay media to 400 million cells/mL and 10,000 cells, or 25 uL/well, were added to assay plates. Variant samples were titrated in triplicate at 1:5 dilution in 25 ul directly in 384-well black flat bottom assay plates (ThermoFisher, Watham, Mass.). Recombinant human IL12 (Peprotech, Rocky Hill, N.J.) was included as a positive control. Plates were incubated for 3 days at 37° C. and 5% carbon dioxide. Post incubation, 25 uL/well of supernatant was transferred to non-binding 384-well plates (Greiner-Bio-One, Kremsmünster, Austria) and stored at −80° C.

Relative Cell Abundance Determination: After supernatant removal, CellTiter-Glo® Luminescent Cell Viability reagent (Promega, Madison, Wis.) was added to plates at 25 uL/well and plates were incubated at room temperature away from light for 30 minutes. Following incubation, plate luminescence was scanned on the BioTek synergy H1 plate reader (BioTek, Winooski, Vt.). Graphs were generated using GraphPad Prism version 7.0d for Mac OS X (GraphPad Software, La Jolla Calif. USA).

Results

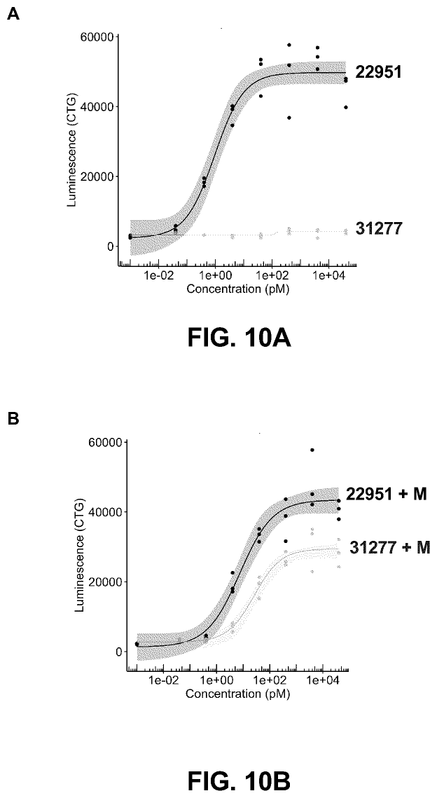

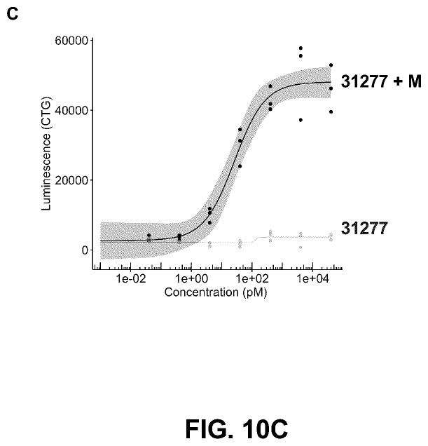

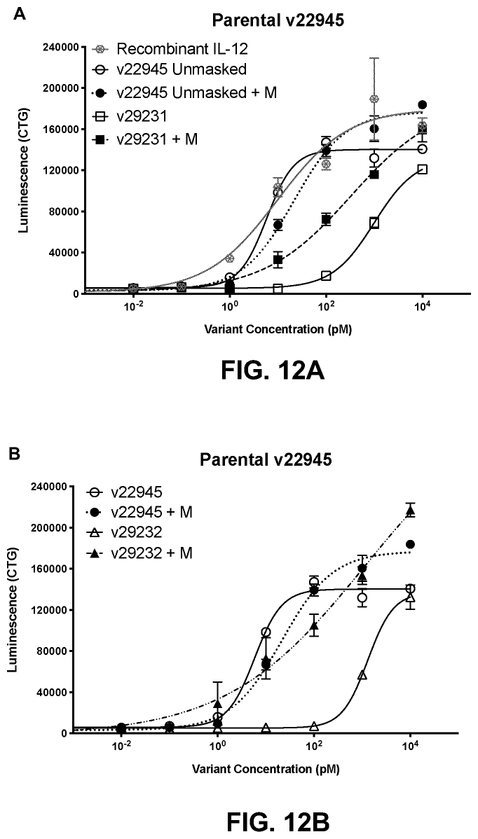

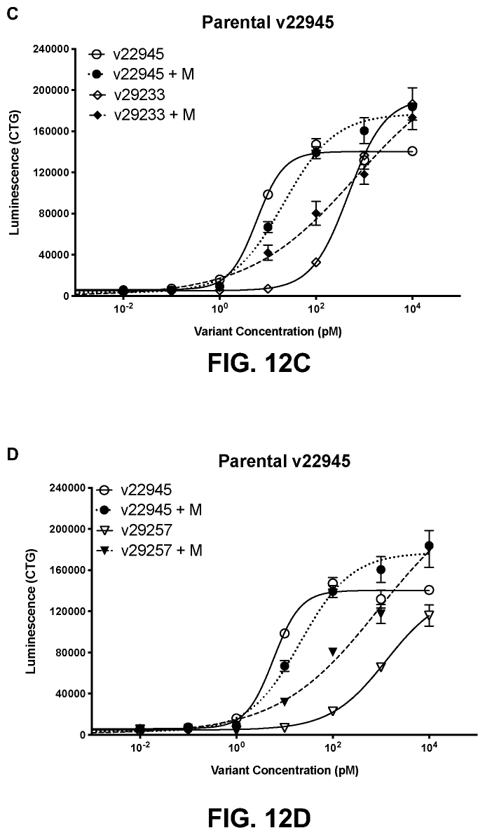

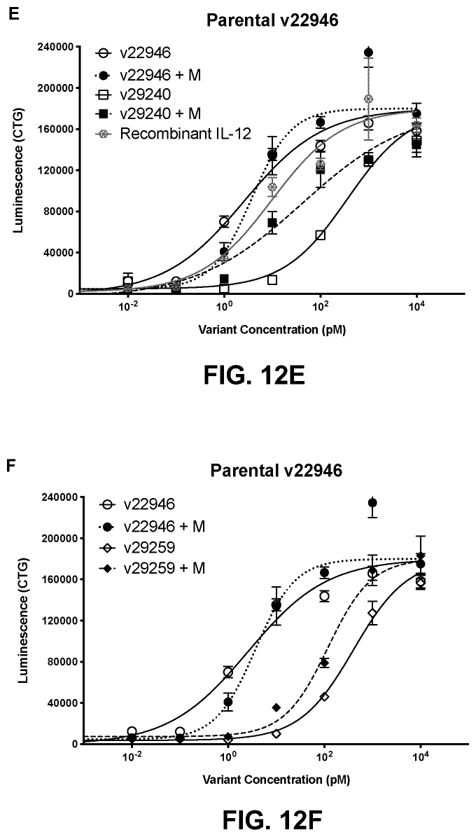

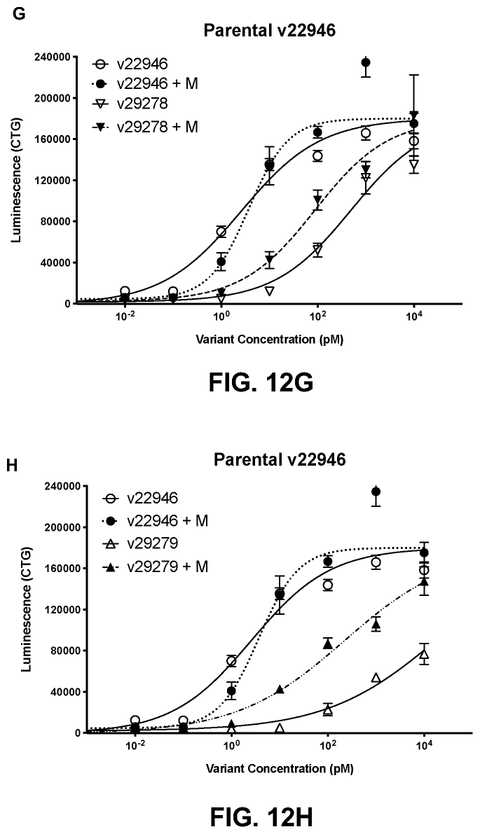

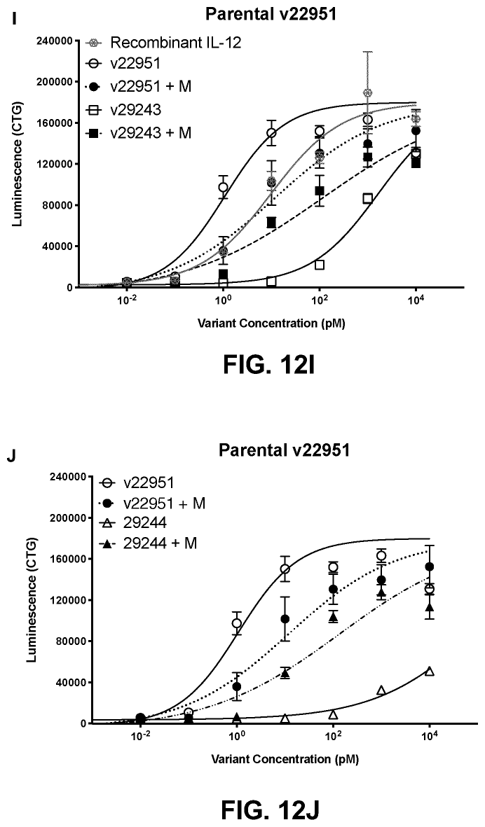

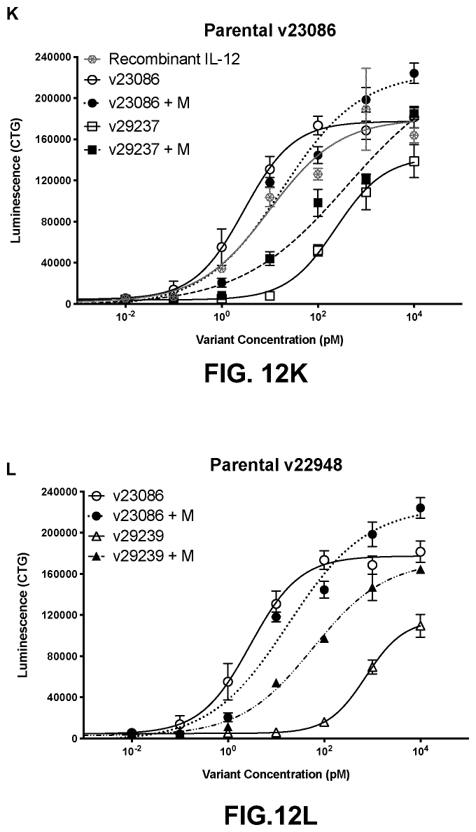

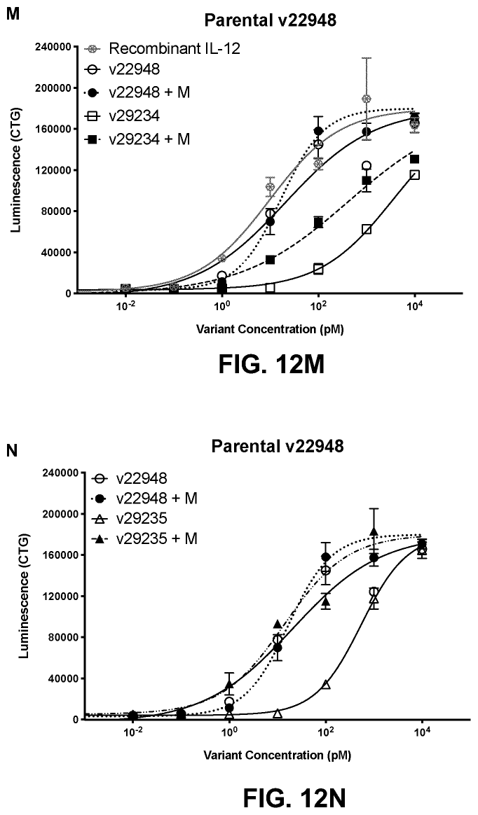

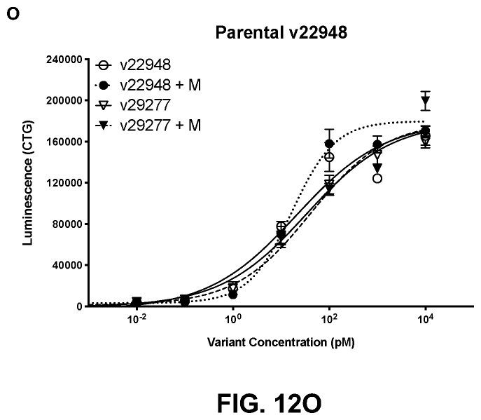

The relative abundance of NK cells after incubation in the presence of masked and parental IL12 HetFc fusion proteins treated+/−matriptase are shown in FIG. 10A-FIG. 15E and summarized in Table AA.

Parental non-masked IL12 HetFc fusion proteins had potencies within < or >10-fold of recombinant IL12 on relative NK cell abundance. Matriptase treatment of parental variants reduced their potency by no more than 6-fold compared to recombinant IL12. Antibody and receptor masked IL12 HetFc fusion proteins showed reduced activity on relative NK cell abundance compared to their corresponding non-masked parental variants (FIG. 10A-FIG. 15E).

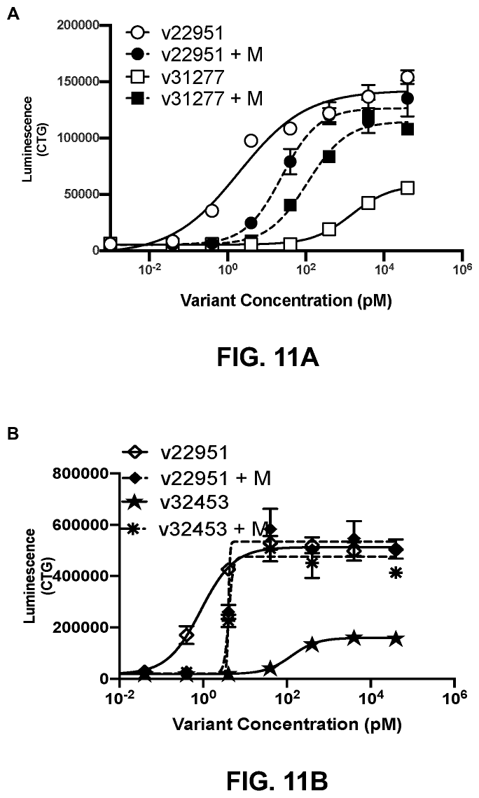

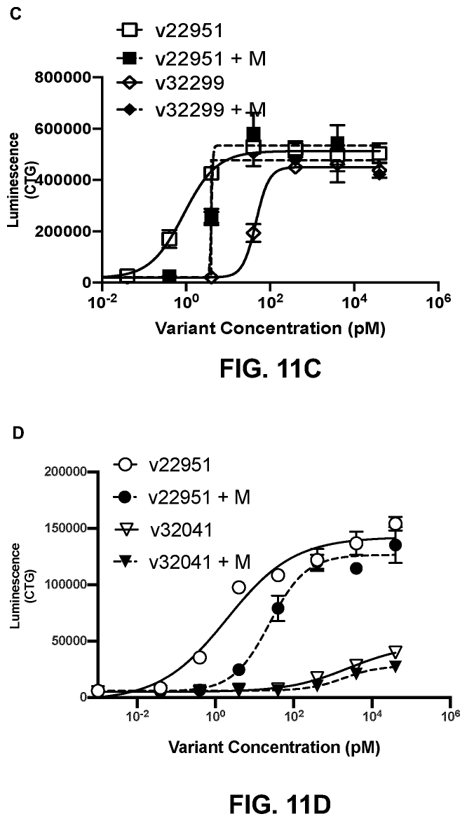

The maximum reduction in potency on relative cell abundance was observed with antibody masked variants v31277 and v32453 compared to their common parental variant v22951. Variant v31277 possesses a first cleavage site between the HetFc and the scFv mask and a second cleavage site between the scFv VH and VL. In a first experiment using v31277 produced from Expi293™ culture, the sample showed an almost complete reduction in potency compared to parental variant v22951, and recovered potency to within 4-fold of v22951 upon matriptase treatment (FIGS. 10A-10C). In a second experiment using v31277 produced from a CHO-3E7 culture, the sample showed a 743-fold reduction in potency compared to v22951 and recovered potency to within 4-fold of v22951 upon matriptase treatment (FIG. 11A). The difference in masking efficiency between these samples is likely attributable to pre-cleavage of this variant between the scFv VH and VL domains that was observed during production in CHO-produced but not HEK-produced samples, as described in Example 7. In comparison, variant v32453 possesses a cleavage site only between the HetFc and scIL12, which does not display any pre-cleavage when produced in CHO culture, and displayed an 147-fold reduction in potency compared to v22951 and recovered equivalent potency to 22951 after matriptase treatment (FIG. 11B). Variant v32299 is identical to v31277 but includes the H_Y32A mutation that weakens the scFv mask affinity (KD) for IL12 by ˜146-fold, as described in Example 4. When produced in CHO-3E7, v32299 showed pre-cleavage between the scFv VH and VL similar to v31277, and displayed a 53-fold reduction in potency on relative NK cell abundance compared to v22951 and recovered equivalent potency to 22951 after matriptase treatment (FIG. 11C). The control variant v32041, identical to v31277 but lacking protease cleavage motifs, demonstrated a 1238-fold reduction in potency compared to v22951 and, as expected, a minimal potency shift when pre-treated with matriptase (FIG. 11D). The maximum reduction in potency of an antibody masked variant derived from a parental non-masked variant other than v22951 was 317-fold for v29279, which was derived from parental v22946. After matriptase treatment, IL12 activity potency was recovered within 18-fold of matriptase-treated v22946 (Error! Reference source not found.12H).

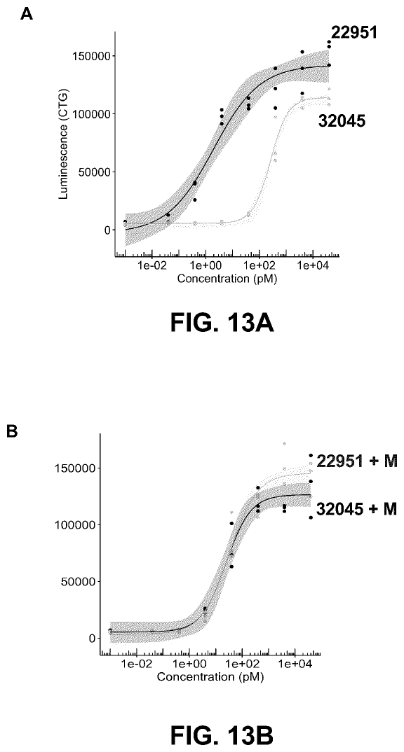

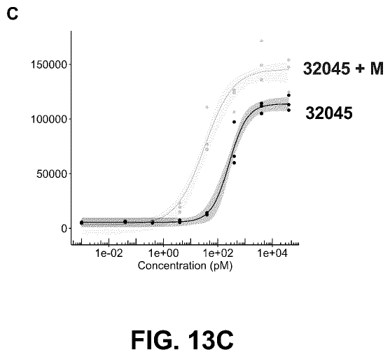

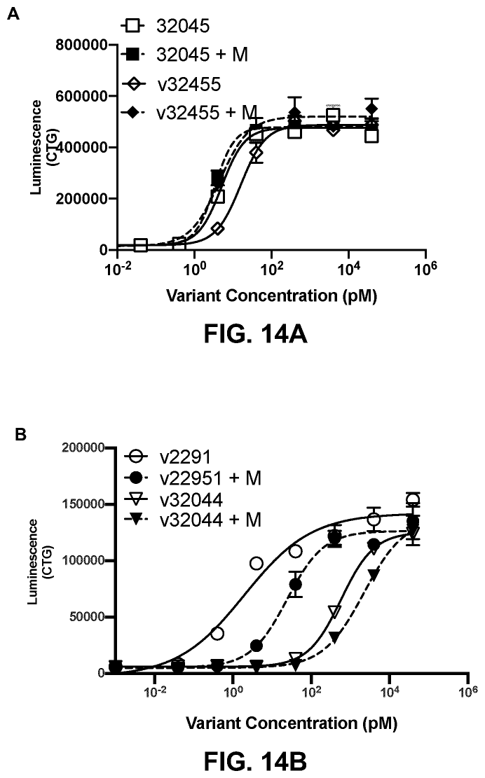

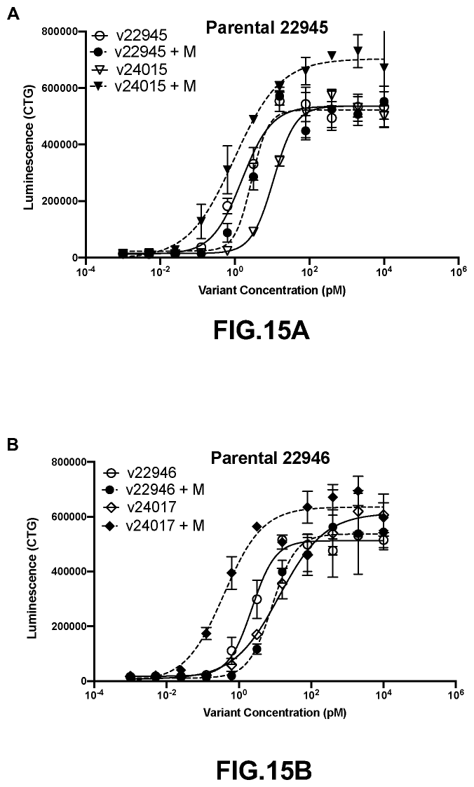

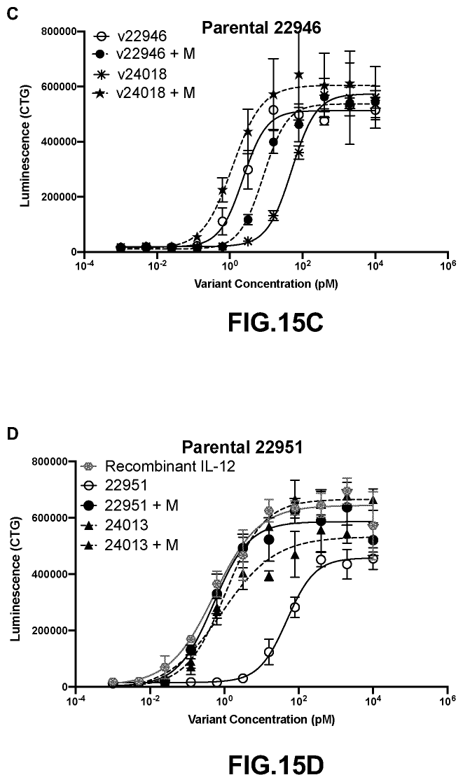

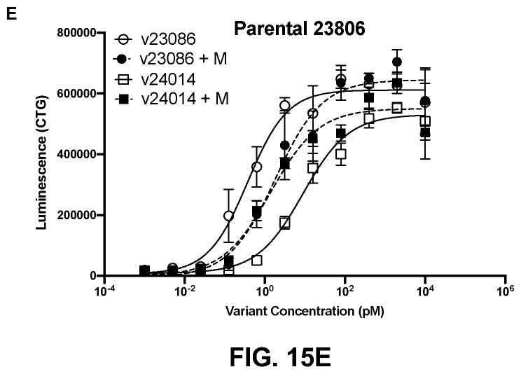

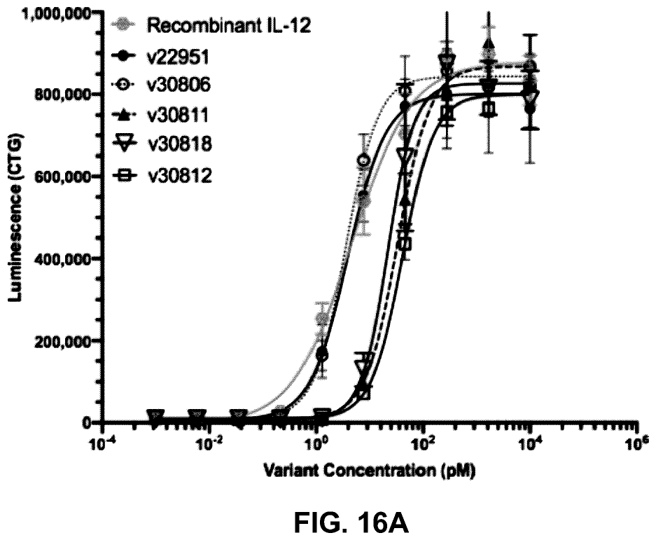

Among receptor-masked variants, the maximum reduction in potency on relative cell abundance was observed for variants v32045 and v32455 compared to their parental variant v22951. These variants differ in the placement of the matriptase cleavage site, which is between the HetFc and the receptor mask for v32045, and between the HetFc and scIL12 for v32455. In one experiment, v32045 displayed 133-fold reduced potency compared to v22951 (FIGS. 13A-13C), and in a second experiment, v32455 showed 3-fold reduced potency compared to v32045 (FIG. 14A). Both variants recovered potencies comparable to v22951 after matriptase treatment. In this case, neither variant displayed observable pre-cleavage, so the improved masking of v32455 compared to v32045 may be due to its longer linker between the HetFc and scIL12 allowing a more stable formation of the masked complex. The control variant v32044, identical to v32045 but lacking cleavage motifs, demonstrated a 295-fold reduction in potency compared to v22951 and, as expected, a minimal potency shift when pre-treated with matriptase (FIG. 14B). The maximum reduction in potency of a receptor-masked variant derived from a parental non-masked variant other than v22951 was 24-fold for v24014, which was derived from parental v23 806. After matriptase treatment potency was recovered to approximately 4-fold above matriptase-treated v23086 (FIG. 15E).

The range of masking efficiencies observed for variants that differ only in fusion configuration (e.g. excluding mutations that modulate mask affinity, cytokine potency, etc.) demonstrates the importance of geometry in constructing masked cytokine fusion proteins. While it is known in the art that the configuration of fusion proteins may impact production efficiency and stability, it is not predictable how configuration affects these characteristics, nor is it guaranteed that they correlate to the desired function of the purified product. Despite all fusions in this work being constructed with linkers designed to sufficiently bridge distances between termini of component domains based on structural analyses, there were differences in masking efficiencies even among variants with similar biophysical characteristics. It is evident that the sequence of fusions between masks, cytokines, and HetFc can have unexpected or unpredictable impacts on function, which may be due to more complex conformational dynamics causing strain or non-specific interactions between the component protein domains or linkers.

TABLE AA
Fold potency reduction and IL12 activity recovery-
Relative Abundance of NK Cells

| | | | | | Variant | Var. +M | |
|---|---|---|---|---|---|---|---|
| | | | | | EC50/Parental | EC50/Parental | |
| | | | | | Variant EC50 | Var. +M EC50 | |
| | | | | | Fold Change in | X Change in IL12 | |
| | | | | Heparin | Potency compared | activity recovery | |
| | Unmasked | | Cleavage Site | Binding | to Parental | post cleavage vs | |
| Variant | Parental | Mask Type | Location (—/—) | Mutation | Variant | Parental Var. | FIG. |
| 29232 | 22945 | ScFv | HetFc-p35—/—Mask | | 222 | 62 | 12B |
| 29257 | 22945 | ScFv | Mask—/—p40 | | 194 | 31 | 12D |
| 29231 | 22945 | ScFv | p40—/—Mask | | 168 | 14 | 12A |
| 29233 | 22945 | ScFv | HetFc—/—Mask | | 76 | 32 | 12C |
| 24015 | 22945 | Receptor | HetFc—/—Mask | | 7 | 0.35 | 15A |
| 29279 | 22946 | ScFv | Mask—/—p35-HetFc | | 317 | 18 | 12H |
| 29259 | 22946 | ScFv | Mask—/—p40 | | 184 | 29 | 12F |
| 29240 | 22946 | ScFv | p40—/—Mask | | 125 | 2 | 12E |
| 29278 | 22946 | ScFv | Mask—/—HetFc | | 113 | 21 | 12G |
| 24018 | 22946 | Receptor | Mask—/—p35-HetFc | | 22 | 0.14 | 15C |
| 24017 | 22946 | Receptor | Mask—/—HetFc | | 6 | 0.05 | 15B |
| 29234 | 22948 | ScFv | Mask—/—p40-HetFc | | 56 | 5 | 12M |
| 29235 | 22948 | ScFv | p35—/—Mask | | 31 | 7 | 12N |
| 29277 | 22948 | ScFv | Mask—/—HetFc | | 1 | 4 | 12O |
| 32039 | 22951 | ScFv | HetFc—/—Mask, | (+) | <10000 | 8 | 17A |
| | | | Mask (VH—/—VL) | | | | |
| 32040 | 22951 | ScFv | No Cleavage | (+) | <10000 | NC | 17B |
| 32454 | 22951 | ScFv | HetFc—/—scIL12 | (+) | <10000 | 6 | 17C |
| 31277 | 22951 | ScFv | HetFc—/—Mask, | | <10000 | 4 | 10 |
| | | | Mask (VH—/—VL) | | | | |
| 32042 | 22951 | Receptor | HetFc—/—Mask | (+) | 1595 | 1.81 | 17D |
| 32043 | 22951 | Receptor | No Cleavage | (+) | 1583 | 127 | 17E |
| 32041 | 22951 | ScFv | No Cleavage | | 1238 | 77 | 11D |
| 29244 | 22951 | ScFv | HetFc—/—Mask | | 1022 | 3 | 12J |
| 29243 | 22951 | ScFv | HetFc-scIL12—/—Mask | | 867 | 3 | 12I |
| 31277 | 22951 | ScFv | HetFc—/—Mask, | | 743 | 4 | 11A |
| | | | Mask (VH—/—VL) | | | | |
| 32044 | 22951 | Receptor | No Cleavage | | 295 | 91 | 14B |
| 32453 | 22951 | ScFv | HetFc—/—scIL12 | | 147 | 1 | 11B |
| 32045 | 22951 | Receptor | HetFc—/—Mask | | 133 | 1 | 13 |
| 24013 | 22951 | Receptor | HetFc—/—Mask | | 94 | 0.78 | 15D |
| 32299 | 22951 | ScFv | HetFc—/—Mask, | | 53 | 1 | 11C |
| | | | Mask (VH—/—VL) | | | | |
| 32455 | 22951 | Receptor | HetFc—/—scIL12 | | 18 | 1 | 14A |
| 30812 | 30806 | No Mask | No Cleavage | (+) | 11 | | 16A |
| 30811 | 30806 | No Mask | No Cleavage | (+) | 9 | | 16A |
| 30816 | 30806 | No Mask | No Cleavage | (+) | 7 | | 16B |
| 30818 | 30806 | No Mask | No Cleavage | (+) | 6 | | 16A |
| 32045 | 22951 | Receptor | HetFc—/—Mask | | 6 | 1 | 14A |
| 30815 | 30806 | No Mask | No Cleavage | (+) | 5 | | 16B |
| 30814 | 30806 | No Mask | No Cleavage | (+) | 3 | | 16B |
| 30813 | 30806 | No Mask | No Cleavage | (+) | 2 | | 16B |
| 30806 | 22951 | No Mask | No Cleavage | | 1 | | 16A |
| 29239 | 23806 | ScFv | p35—/—Mask | | 236 | 3 | 12L |
| 29237 | 23806 | ScFv | HetFc—/—Mask | | 67 | 1 | 12K |
| 24014 | 23806 | Receptor | HetFc—/—Mask | | 24 | 1 | 15E |

These results suggest that parental non-masked IL12 HetFc fusions have activity within a similar potency range to recombinant IL12, and that ScFv or receptor masked IL12 HetFc fusions: 1) attenuate or block IL12 activity; 2) recover IL12 activity when cleaved by proteases, and 3) can be modified to alter the efficiency of the mask and recovery of IL12 activity.

### EXAMPLE 10: SEQUENCES OF IL12 WITH REDUCED AFFINITY FOR HEPARIN

IL12 can be purified by heparin-affinity chromatography (Jayanthi et al. Protein Ex Purif 2014; 102:76-84) and the presence of heparin, a negatively charged sugar polymer, enhances its in vitro activity (Jayanthi et al. Scientific Reports 2017). A positively charged loop of sequence QGKSKREKK in the IL12 p40 subunit is likely responsible for binding heparin (see SEQ ID NO:19 and amino acids 256-264 of SEQ ID NO:22). In this Example, residues within this loop were mutated or replaced with loops of shorter length and various net charges to lower the binding affinity of IL12 to heparin and attenuate the potency of IL12. In addition, the mutants may provide resistance to cleavage by matriptase, which was observed within this loop as described in Example 8, and may improve pharmacokinetics due to reduced non-specific membrane binding.

TABLE 12
Heparin-binding loop sequences of IL12 p40

| | HetFc 1 | HetFc 2 | p40 heparin | SEQ |
|---|---|---|---|---|
| Variant | clone | clone | binding | ID |
| ID | ID | ID | loop sequence | NO: |
| v30806 | CL #22279 | CL #12153 | QGKSKREKK | 19 |
| v30811 | CL #22296 | CL #12153 | QGSEK | 244 |
| v30812 | CL #22295 | CL #12153 | KDQTE | 245 |
| v30813 | CL #22294 | CL #12153 | QDDSE | 246 |
| v30814 | CL #22293 | CL #12153 | QDQTD | 247 |
| v30815 | CL #22292 | CL #12153 | QGEKK | 248 |
| v30816 | CL #22289 | CL #12153 | RDDSE | 249 |
| v30817 | CL #22290 | CL #12153 | QGSQEKK | 250 |
| v30818 | CL #22291 | CL #12153 | QGESKQEKK | 251 |

Methods

Non-masked IL12 HetFc fusions were designed based on parental variant v22951 with mutations in the heparin binding loop (Table 12), produced in Expi293™ as described in Protocol 2, and purified by pA and SEC as described in Protocol 7 and Protocol 8.

The p35 sequence used for the scIL12 sequences containing the loop grafts had the N-terminal arginine removed and started with Asn2, to prevent cleavage between the Gly-Ser linker and p35 N-terminus as described in Example 8. Variant v30806 contains only this modification as compared to parental variant v22951 and contains the wild type heparin binding loop.

Variants were assessed by UPLC-SEC post pA as described in Protocol 10 for their percentage of high molecular weight species, and melting temperatures (Tm) were determined by DSC as described in Protocol 11.

Variants were tested for susceptibility to matriptase cleavage as described in Example 8, with additional digest timepoints assessed by reducing LabChip™ CE-SDS at 1h and 6h.

Heparin binding of variants was assessed by injecting 0.2 mg of sample on a 1 mL heparin HiTrap Column (GE Healthcare) with running buffer 10 mM NaPhosphate, pH 7.4, followed by a wash step for 5 column volumes (CV) and elution in running buffer supplemented with a linear gradient of 0 to 1 M NaCl over 30 CV. The affinity of variants for heparin was compared by measuring the percentage of protein in the elution peak vs. percentage of protein in the flow through based on A280, as well as by comparing the elution column volume.

The relative abundance of NK cells treated with variants containing mutated heparin binding loops was assessed as described in Example 9.

Results

Table 13 shows results for pA yield per L of cell culture, biophysical properties, and heparin column binding characteristics of variants with mutated heparin binding loops. All variants exhibited WT stability and yields post pA compared to v30806. All variants exhibited decreased binding affinity to the heparin column, evident either by their earlier elution CV compared to the WT v30806, which eluted at 25.5 mL CV, or by their percentage of protein that did not bind to the column and remained in the flow through. For example, v30812 eluted at 17.2 mL CV and only 58.5% of the protein loaded was eluted from the column during the salt gradient, 41.5% of protein did not bind and remained in flow through and thus did not bind to heparin. The variants displayed varying resistance to matriptase digestion, up to complete resistance to 24h incubation with matriptase. Variant v30806 displayed complete cleavage at 1h, variants v30811 through v30816 displayed no cleavage up to 24h, and variants v30817 and v30818 displayed increasing cleavage beginning at 1h and proceeding to near completion at 24h. Variants did not display banding corresponding to cleavage at the N-terminus of p35 as described in Example 8 for variants that do possess Arg 1 of p35.

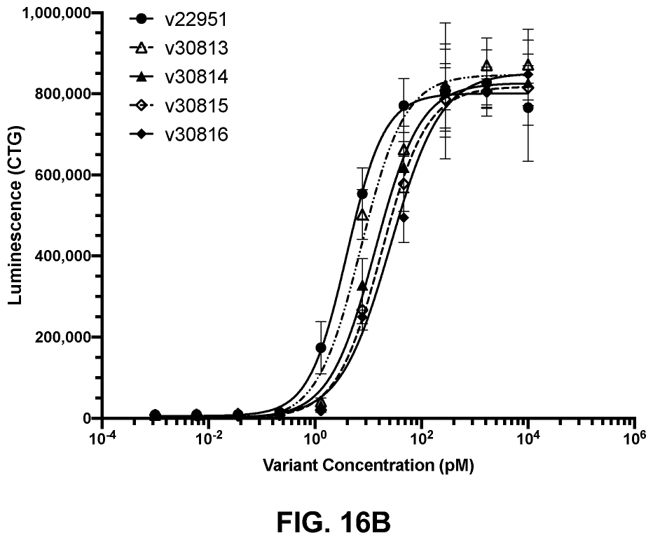

The relative abundance of NK cells after incubation in the presence of heparin binding mutant IL12 HetFc fusion proteins is shown in FIGS. 16A-16B and is summarized in Table AA. Variants 22951 and 30806 had equivalent potency on relative abundance of NK cells, indicating that removal of the N-terminal arginine from variant 22951 to create variant 30806 did not affect activity (FIG. 16A). Introduction of heparin binding mutations resulted in maximum attenuation of potency of 11-fold for variant 30812 compared to 30806 whereas other variants showed potency attenuation between 2 to 9-fold (FIG. 16). Thus, while there was some reduction in IL12 activity observed by introduction of mutations in the heparin binding site, given the high potency and toxicity of IL12, this reduction may be considered acceptable in order to further reduce the potency of masked IL12 fusions.

TABLE 13
Yield, biophysical properties, and
heparin column binding of mutants

| | | UPLC-SEC | | Heparin | A280 |
|---|---|---|---|---|---|
| Variant | pA yield per L | HMW | Tm | Elution | Elution/ |
| ID | culture (mg) | post pA (%) | (° C.) | CV (mL) | FT (%) |
| v30806 | 384.4 | 9.4 | 64.9 | 25.5 | 96.4 |
| v30811 | 478.4 | 19.4 | 64.8 | 18.4 | 90.9 |
| v30812 | 416.4 | 7.4 | 65.7 | 17.2 | 58.5 |
| v30813 | 450.0 | 28.2 | 65.0 | 16.3 | 10.0 |
| v30814 | 420.4 | 19.3 | 65.2 | 17.4 | 41.2 |
| v30815 | 398.8 | 25.2 | 63.1 | 19.8 | 89.7 |
| v30816 | 368.4 | 29.8 | 64.6 | 17.1 | 18.4 |
| v30817 | 371.6 | 18.8 | 65.2 | 19.5 | 98.3 |
| v30818 | 449.6 | 7.3 | 65.4 | 19.5 | 96.2 |

### Example 11: Design, Production and Testing of Masked IL12 HetFc Fusion Proteins with Reduced Affinity for Heparin

To determine the effect of a mutated heparin loop and associated IL12 attenuation on the potency of masked IL12 HetFc fusions proteins, the mutated heparin loop sequence from v30818 (Table 12) was applied to select masked variants, and proteins were produced and tested for their effects on NK cell relative abundance.

Antibody and Receptor-masked IL12 HetFc fusion proteins were designed as described in Examples 5 and 6, where the variants v32039, v32040, v32454, v32042, and v32043 below (Table 14) are equivalent to variants v31277, v32041, v32453, v32045, and v32044, respectively, but with p40 heparin-binding loops modified as in v30818.

TABLE 14
Masked IL12 HetFc fusion proteins with heparin loop mutations

| | | |
|---|---|---|
| Variant ID | HetFc 1 clone ID | HetFc 2 clone ID |

Briakinumab-masked IL12 HetFc fusion
proteins derived from parental v22951

| | | |
|---|---|---|
| v32039 | CL_#22735 | CL_#22291 |
| v32040 | CL_#23512 | CL_#22291 |
| v32454 | CL_#23512 | CL_#23711 |

IL12Rβ2-masked IL12 HetFc fusion
proteins derived from parental v22951

| | | |
|---|---|---|
| v32042 | CL_#22672 | CL_#22291 |
| v32043 | CL_#23513 | CL_#22291 |

### Methods

Proteins were produced and characterized as described in Example 7, tested for matriptase cleavage as described in Example 8, and tested for NK cell activity as described in Example 9.

### Results

Yields and UPLC-SEC purity of masked variants containing the heparin loop mutations were comparable to the corresponding variants with wild-type heparin binding loops as described in Example 7. Variant v32039, which contains a second matriptase cleavage motif between the scFv VH and VL like v31277, also displayed a small amount of pre-cleavage as shown by reducing LabChip™ CE-SDS analysis, corresponding to 1.3% of the total HetFc-mask protein chain. All variants were fully cleaved by overnight treatment with matriptase as described in Example 8.

The relative abundance of NK cells after incubation in the presence of masked IL12 HetFc fusion proteins with heparin loop mutations treated+/−matriptase are summarized in FIGS. 17A-17E and Table AA. In general, variants with heparin loop mutations displayed similar masking and unmasking behavior to the corresponding variants with wild-type heparin loops but with overall decreased potency, as expected based on the reduced potency of the non-masked variant v30818 with a mutated heparin loop compared to v30806 with the wild-type loop (FIGS. 16A-16B).

The variant v32039, identical to v31277 but containing the heparin loop replacement, demonstrated a close to complete reduction in potency compared to the corresponding non-masked parental variant with a wild-type heparin binding loop, v22951, and recovered to within 8-fold of v22951 potency when pre-treated with matriptase (FIG. 17A).

The variant v32040, identical to v32041 (lacking a protease cleavage site) but containing the heparin loop replacement, demonstrated a close to complete reduction in potency compared to v22951 and, as expected, a minimal potency shift when pre-treated with matriptase (FIG. 17B).

The variant v32454, identical to v32453 (cleavage site only between HetFc and scIL12) but containing the heparin loop replacement, demonstrated a complete reduction in potency and recovered to within 6 fold of v22951 potency when pre-treated with matriptase (FIG. 17C).

The variant v32042, identical to v32045 but containing the heparin loop replacement, demonstrated a 1595-fold reduction in potency compared to v22951 and recovered to within 2-fold of v22951 potency when pre-treated with matriptase (FIG. 17D).

The variant v32043, identical to v32044 (lacking a protease cleavage site) but containing the heparin loop replacement, demonstrated an 1583-fold reduction in potency compared to v22951 and, as expected, a minimal potency shift when pre-treated with matriptase (FIG. 17E).

These data demonstrate that heparin binding loop mutations reduce the potency of masked IL12 HetFc variants compared to their corresponding variants having wild-type heparin binding loops by a greater amount in the masked form than in the non-masked form after cleavage. Thus, antibody and receptor masks function synergistically with IL12 attenuation in the context of IL12 HetFc fusion proteins to broaden the potency shift before and after mask removal by protease cleavage.

### Example 12: CD8+ T-Cell IFNγ Release after Incubation with IL12 HetFc Fusion Proteins+/−Matriptase

In addition to NK cells, CD8+ T cells are an important target population for IL12. The potency of select variants derived from the parental variant v22951 on CD8+ T cells was assessed by IFNγ release.

Methods

CD8+ T Cell Assay: CD8+ T cells were thawed, stimulated with anti-CD3/CD28 dynabeads (ThermoFisher, Waltham, Mass.) at a cell to bead ratio of 10:1, and plated in 384-well black flat bottom assay plates (ThermoFisher, Watham, Mass.) at 30,000 cells/well in 30 ul RPMI1640 (Gibco)+10% FBS (ThermoFisher)+1% Pen-Strep (Gibco). Plates were incubated overnight at 37° C. and 5% carbon dioxide. The following day, samples were prepared as below and 30 ul were added to CD8+ T cells. Plates were incubated for 3 days at 37° C. and 5% carbon dioxide. Post incubation, 15 uL/well of supernatant was transferred to non-binding 384-well plates (Greiner-Bio-One, Kremsmünster, Austria) and stored at −80° C.

Sample preparation: 2 aliquots of variant or control samples were thawed from −80° C. storage the day prior to the assay. Recombinant human matriptase was added to a single aliquot of each sample (R&D Systems, Minneapolis, Minn.) at a 50:1 sample to enzyme ratio and vortexed to mix. Samples were titrated in triplicate at 1:20 dilution in 100 ul in non-binding 384-well plates (Greiner-Bio-One, Kremsmünster, Austria). Recombinant human IL12 (Peprotech, Rocky Hill, N.J.) was included as a positive control. 30 ul of titrated variants were then transferred to simulated CD8+ T cells as above.

IFNγ Quantification: IFNγ was quantified using MSD (Mesoscale Discovery, Piscataway, N.J.). The night before cytokine quantification, MSD plates were blocked and coated in capture antibodies according to the manufacturers' instructions. The following day, plates were washed in PBS-T and 5 ul of assay diluent was added to each plate. The supplied IFNγ standard was titrated from 1000 ng/mL down to 1 pg/mL. Supernatants were thawed at room temperature and 5 uL of samples or standards were transferred to MSD plates. Detection antibodies were prepared at appropriate dilutions and 10 uL was added to each sample and standard well in MSD plates. The plates were sealed with aluminum foil and incubated away from light at room temperature for two hours. Plates were washed 3× in PBS-T and 40 uL MSD Gold read buffer T was added to each well. Plates were read on the MESO SECTOR 6000 and cytokine concentration was determined using MSD software. Data from a standard curve and samples were used to perform a nonlinear curve-fit with x-interpolation to obtain IFNγ concentrations in pg/mL. Four independent experiments were conducted and data from each was analyzed in a nonlinear mixed effect model to generate curve fit and 95% confidence intervals.

Results

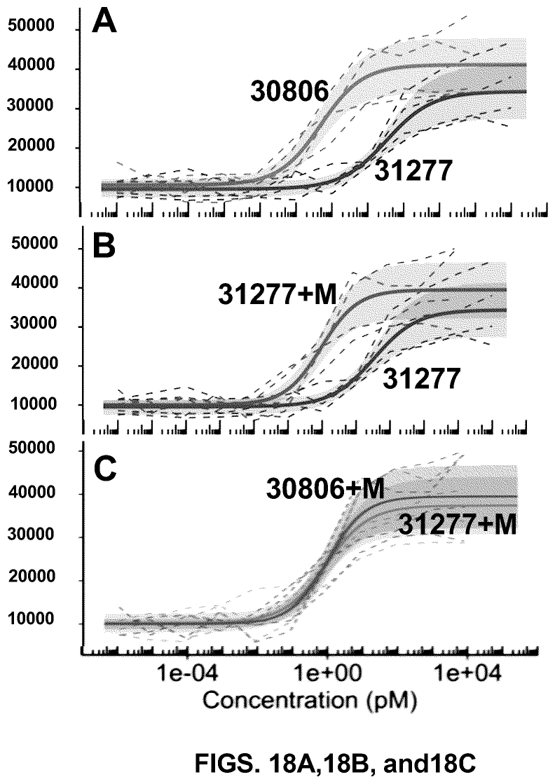

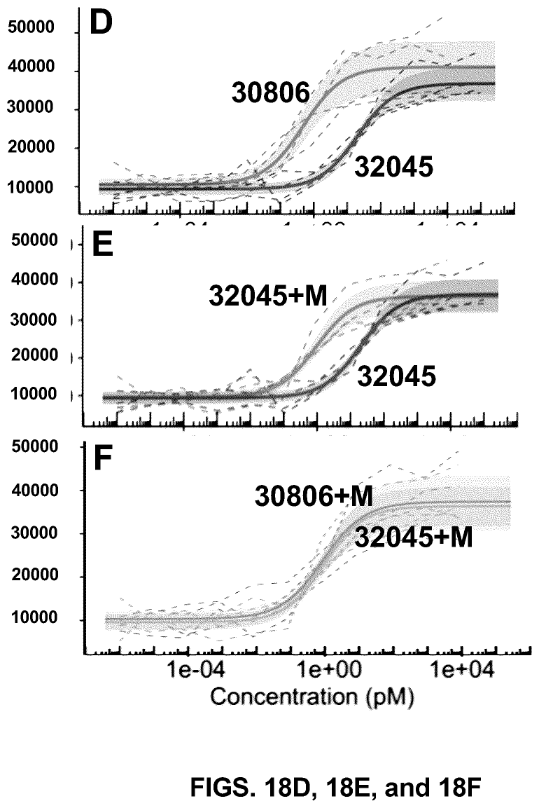

CD8+ T cell IFNγ release after incubation in the presence of the non-masked IL12 HetFc fusion variant v30806 (equivalent to parental v22951 but with the N-terminal Arg of p35 removed) and masked variants derived from v22951 treated+/−matriptase are summarized in FIGS. 18A-18F and Tables 10 and BB.

Across four independent experiments, antibody and receptor masked variants induced significantly less IFNγ release compared to non-masked IL12 HetFc variant v30806. The potencies of antibody masked variant v31277 and receptor masked variant v32045 were reduced 69-fold (p=0.00051) and 41-fold (p<10⁻⁶) compared to v30806, respectively (FIGS. 18A and 18D). Pre-treatment of masked variants with matriptase resulted in recovery of IFNγ release by 35-fold for variant v31277 (p<10⁻⁶) and 21-fold (p<10⁻⁶) for variant v32045 (FIGS. 18B and 18E). Matriptase treated antibody and receptor masked variant potencies were not significantly different than matriptase treated parental variants (FIGS. 18C and 18F). v32862, which is derived from v31277 but with an alternate non-cleavable linker between the Briakinumab scFv VH and VL domains, displayed a 52-fold reduction in potency compared to non-masked v30806 (FIG. 18G).

TABLE BB
Fold potency reduction and IL12 activity recovery- IFNγ
production by CD8T Cells

| | | | | | | Var. +M | |
|---|---|---|---|---|---|---|---|
| | | | | | | EC50/Parental | |
| | | | | | Variant | Var. +M EC50 | |
| | | | | | EC50/Parental | Fold Change in | |
| | | | | | Variant EC50 | IL12 activity | |
| | | | | Heparin | Fold Change in | recovery post | |
| | Unmasked | Mask | Cleavage Site | Binding | Potency compared | cleavage vs | |
| Variant | Parental | Type | Location | Mutation | to Parental Variant | Parental Var | FIG. |
| 31277 | 30806 | ScFv | HetFc—/—Mask, Mask | | 69 | 1 | 18A-C |
| | | | (VH—/—VL) | | | | |
| 32045 | 30806 | Receptor | HetFc—/—Mask | | 41 | 1 | 18D-F |

### Example 13: In Vivo Activity of Parental IL12 Fusion Proteins

Recombinant IL12 is severely toxic in humans and mice when administered systemically. We developed an in vivo model to assess tolerability of IL12 HetFc fusion proteins utilizing severely immunocompromised NOG mice engrafted with human PBMCs.

Methods:

Two cohorts of 4-5 week old NOG mice were injected intravenously with 1×10⁷ human PBMCs (thawed from frozen) from two donors. One day post engraftment, mice were administered parental, non-masked IL12 HetFc fusion variants v30806 and v30818 intraperitoneally at 1 or 5 mg/kg. A second dose of variant was administered on day 8. Body weight and clinical health signs were monitored daily. Mice were euthanized when they reached >20% body weight loss and/or exhibited irreversible worsening of clinical health score. Select mice were bled on days 1, 3, 7 and 9 post initial dose. Serum was isolated from blood collected at all time points and frozen at −80° C. for subsequent pharmacokinetic analysis of variants. Presence of IL12 HetFc variants was assessed using an anti-IL12 p35 antibody capture and anti-human Fc gamma HRP detection sandwich ELISA. Results were analyzed using Graph Pad Prism. Results from survival were analyzed using Graph Pad Prism.

Results:

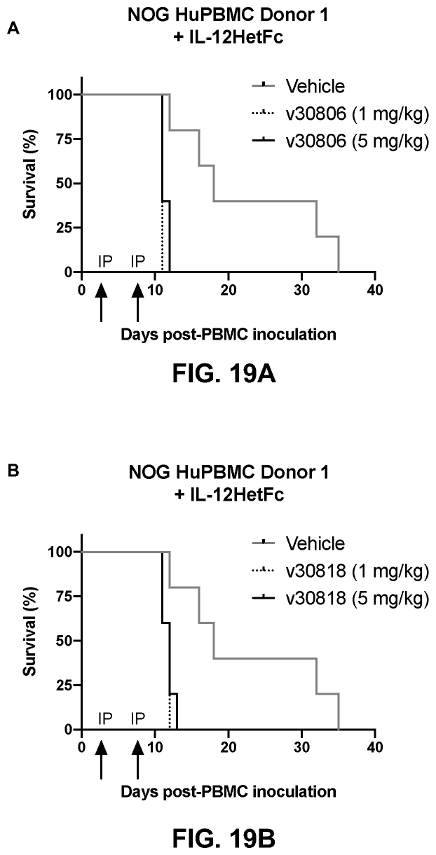

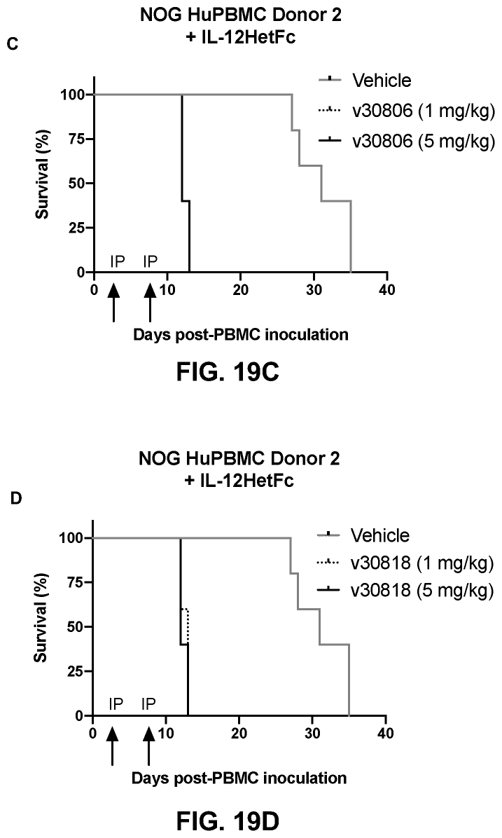

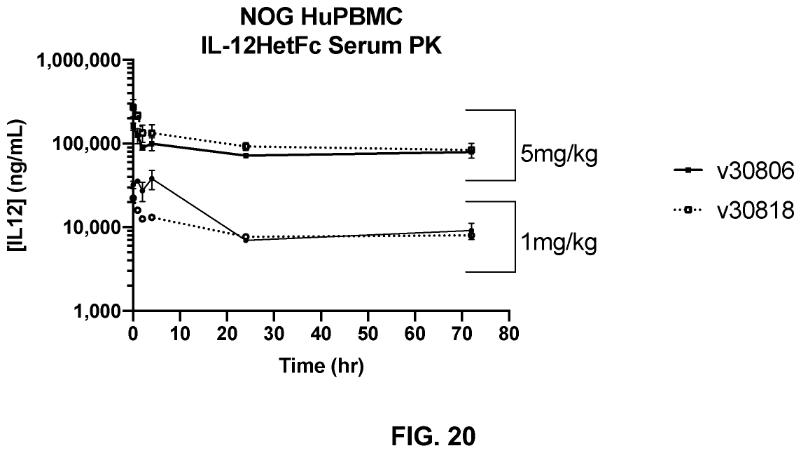

The effects of parental, non-masked IL12 HetFc variants on the survival of mice engrafted with human PBMCs is shown in FIGS. 19A-19D. In both cohorts, a significant decrease in survival was observed within 2 days (experimental day 11) after the second administration of either v30806 or v30818 IL12 HetFc fusions (FIGS. 19A-19D). No difference in survival was observed between mice treated with 1 vs. 5 mg/kg of either variant. No difference in survival was observed between parental non-masked variant v30806, or its counterpart that contains a mutated heparin binding loop, variant v30818, at either dose in either cohort (FIG. 19A vs. FIG. 19B and FIG. 19C vs. FIG. 19D). PK analysis showed that serum levels of v30806 and v30818 were similar at all time points at both the 5 and 1 mg/kg dose, suggesting that mutation of the heparin binding loop did not affect PK as expected (FIG. 20). Overall serum exposure remained high until 3 days, suggesting terminal clearance of IL12 HetFc fusions is slow, which is also unexpected based on serum exposure of other IL12 fusion proteins in the literature. These results indicate that parental, non-masked IL12 HetFc variants have a normal serum exposure and are not tolerated in immunocompromised mice engrafted with human PBMCs at doses above 1 mg/kg. They suggest that masking variants may increase tolerability of IL12 HetFc fusions.

### Example 14: In Vivo Activity of Masked IL12 Fusion Proteins

IFNγ is a key mediator of IL12 dependent toxicity in humans and mice. As masked IL12 HetFc fusion proteins induce significantly less IFNγ production in vitro, they should induce less serum IFNγ in mice, resulting in less toxicity.

Methods: Three cohorts of 4-5 week old NOG mice are injected intravenously with 1×10⁷ human PBMCs (thawed from frozen) from three donors. One day post engraftment, mice are administered parental, non-masked IL12 HetFc or masked IL12 HetFc variants intraperitoneally at doses ranging from 0.0039-1 mg/kg. A second dose of variant is administered on day 8. Body weight and clinical health signs are monitored daily. Select mice are bled on days 1, 3, 7 and 9 post initial dose. Blood is collected at experimental endpoint from all mice. Serum is isolated from blood collected at all time points and frozen at −80° C. for subsequent cytokine and pharmacokinetic analysis of variants.

Results: It is expected that in human PBMC engrafted NOG mice, administration of parental, non-masked IL12 HetFc variants will cause significant loss in body weight and/or deterioration in clinical health signs, as well as increases in serum IFNγ after 1 or 2 administrations of variant. These measures of tolerability are expected to decrease in severity in a dose dependent manner. It is expected that the maximum tolerated dose of masked IL12 HetFc variants will be significantly greater than parental non-masked variants.

### Example 15: Design, Production, and Testing of Double-Masked IL12 HetFc Fusion Proteins

To reduce the IL12 activity of masked IL12 HetFc fusion proteins beyond that achieved with a single masking moiety, multiple masking moieties are incorporated.

### Methods:

To design double-masked IL12 HetFc fusions, two compatible masking moieties (i.e. two non-competing IL12 binding proteins) are fused to one or more available termini of parental non-masked IL12 HetFc fusions via peptide linkers, where either the peptide linker(s) between the IL12 HetFc fusion and the mask(s) and/or between the IL12 HetFc fusion and the IL12 are protease-cleavable. Examples of double-masked variants using a Briakinumab scFv mask in combination with an scFv mask derived from the antibody h6F6 (ref: U.S. Pat. No. 8,563,697 B2), or using a portion of the IL12Rβ1 ECD in combination with a portion of the IL12Rβ2 ECD are listed in Table 15 and diagrammed in FIG. 21.

Proteins are produced and characterized biophysically as described in Example 7, cleaved by matriptase as described in Example 8, and tested for NK or CD8+ T cell activity to assess the reduction in potency of the masked molecules and their recovery of potency post-cleavage as described in Example 9 and Example 12.

TABLE 15
Example double-masked IL12 HetFc fusion proteins

| Variant ID | HetFc 1 clone ID | HetFc 2 clone ID | Other clone ID |
|---|---|---|---|
| v32867 | CL_#22735 | CL_#24228 | |
| v32868 | CL_#24229 | CL_#22279 | |
| v32869 | CL_#24230 | CL_#22279 | |
| v32870 | CL_#24232 | CL_#24231 | |
| v32871 | CL_#24233 | CL_#22279 | |
| v32873 | CL_#24235 | CL_#24236 | CL_#17871 |
| v32895 | CL_#24232 | CL_#24246 | CL_#17871 |
| v35456^{a} | CL_#24224 | CL_#24228 | |
| v35457^{b} | CL_#26503 | CL_#26320 | |

^{a}derived from v32867 but with an alternate non-cleavable linker between the Briakinumab scFv VH and VL domains.
^{b}derived from v35456 but with alternate non-cleavable linkers between the HetFc and Briakinumab scFv VH domains and between the p35 domain and the h6F6 scFv VL domains

### Results:

Following Protein-A purification, only the double-masked variant v32867 was recovered with comparable yield to non-masked or single-masked control variants v30806 and 31277, with yields of 55, 62, and 45 mg/L, respectively, while other double-masked variants had yields of less than 10 mg/L (excluding v35456 and v35457, which were not expressed in this group). UPLC-SEC analysis of PA-purified v32867 revealed 22.4% high molecular weight species, 25.3% correct heterodimeric species, and 52.3% excess single-chain and homodimeric species. In this case, the large amount of excess single-chain and homodimeric species was caused by a non-optimized DNA ratio being used for scale-up. Nevertheless, the desired heterodimeric species was purified subsequently to 94.6% homogeneity by SEC.

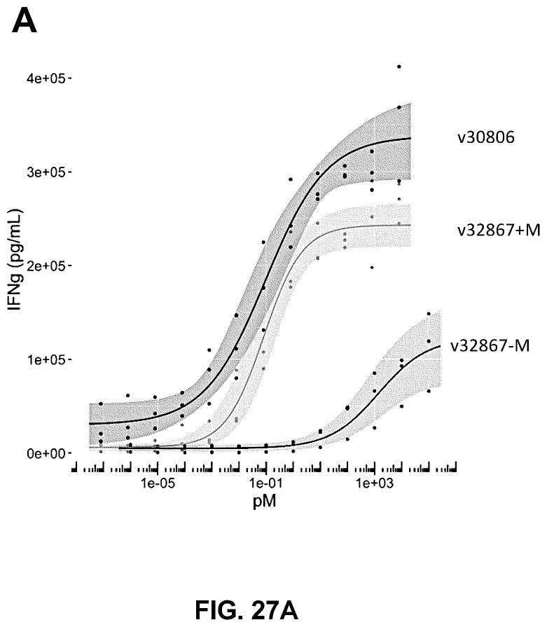

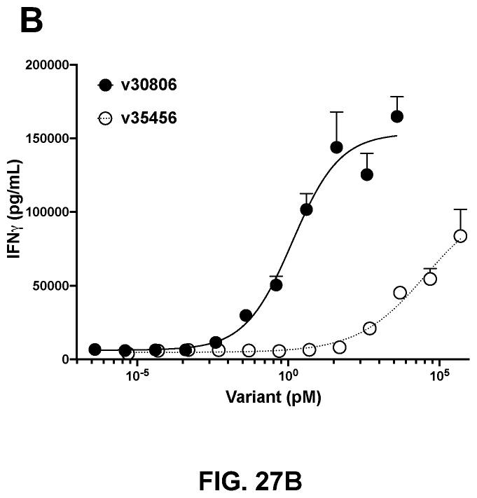

CD8+ T cell IFNγ release after incubation in the presence of the double-masked variant v32867 is shown in FIGS. 27A-27B. Across 3 experiments, v32867 displayed a 14,967-fold reduced potency compared to the corresponding non-masked variant v30806 and a 17,158-fold increased potency after treatment with matriptase (FIG. 27A). v35456, which is derived from v32867 but with an alternate non-cleavable linker between the Briakinumab scFv VH and VL domains, displayed a 25,288-fold reduction in potency compared to non-masked v30806 (FIG. 27B).

### Example 16: MSGRSANA uPa/Matriptase Protease Cleavage Site Tested in Alternative Masked Fusion Protein Format

The cleavage site within v22804 (MSGRSANA; SEQ ID NO:10) was identified as described in Example 2 as a suitable lead cleavage sequence that has high specific cleavage activity for uPA and matriptase, while being resistant to other serine protease such as plasmin. This sequence was used in numerous masked IL12 fusion proteins as described in the Examples above. This example describes the design and construction of a masked anti-CD3 X anti-Her2 T cell engager fusion protein comprising the MSGRSANA protease cleavage site.

An anti-CD3 Fab x anti-Her2 scFv Fc was appended with a mask on the anti-CD3 Fab by linking one of the ligand-receptor pair PD-1-PDL-1 to the N-terminus of the light chain of the Fab and the other to the N-terminus of the heavy chain. The fusion protein constructs were designed as follows.

Methods

The fusion proteins were in a modified bispecific Fab×scFv Fc format with a half-antibody comprising the anti-CD3 heavy and light chain that forms a heterodimer with an anti-Her2 scFv fused to an Fc. The anti-CD3 paratope was described in US20150232557A1 (VL SEQ ID NO: 271; VH SEQ ID NO: 272 (SEQS 1 and 2)). The anti-Her2 paratope was in an scFv format that is based on trastuzumab VL and VH (Carter, P. et al. Humanization of an anti-p185HER2 antibody for human cancer therapy. Proc Natl Acad Sci USA 89, 4285-4289, doi:10.1073/pnas.89.10.4285 (1992)) connected by a glycine serine linker as described in U.S. Ser. No. 10/000,576B1 (SEQ ID NO:273). To allow for selective heterodimeric pairing, mutations were introduced in the anti-CD3 CH3 as well as the anti-Her2 scFv-Fc CH3 chain as described previously (Von Kreudenstein, T. S. et al. Improving biophysical properties of a bispecific antibody scaffold to aid developability: quality by molecular design. MAbs 5, 646-654, doi:10.4161/mabs.25632 (2013); (A chain CH3 domain, SEQ ID NO:274, B chain CH3 domain SEQ ID NO: 275). Mutations (L234A_L235A_D265S as compared to a wild type human IgG1 CH2) were also introduced in both CH2 domains to reduce binding to the Fc gamma receptors (SEQ ID NO: 276). Furthermore, polypeptides based on the modified protein sequences of the IgV domains of human PD-1 (SEQ ID NO: 277) and/or PD-L1 (SEQ ID NO: 278) (West, S. M. & Deng, X. A. Considering B7-CD28 as a family through sequence and structure. Exp Biol Med (Maywood), 1535370219855970, doi:10.1177/1535370219855970 (2019) were fused to the N-termini of heavy chain (VH-CH1-hinge-CH2-CH3) and kappa light chain (VL-CL) of the anti-CD3 variable domains, respectively, using linkers that were comprised of a variable number of repeats of sequences predicted to form helical turns ((EAAAK)ₙ, Chen, X., Zaro, J. L. & Shen, W. C. Fusion protein linkers: property, design and functionality. Adv Drug Deliv Rev 65, 1357-1369, doi:10.1016/j.addr.2012.09.039 (2013)). These PD-1 and PD-L1 moieties were predicted to dimerize and sterically block epitope binding. In all variants, either the PD-1 or the PD-L1 sequence used as one half of the mask contained mutations to increase the affinity of the PD-1:PD-L1 complex as described before (Maute, R. L. et al. Engineering high-affinity PD-1 variants for optimized immunotherapy and immuno-PET imaging. Proc Natl Acad Sci USA 112, E6506-6514, doi:10.1073/pnas.1519623112 (2015); SEQ ID NO: 279; Liang, Z. et al. High-affinity human PD-L1 variants attenuate the suppression of T cell activation. Oncotarget 8, 88360-88375, doi:10.18632/oncotarget.21729 (2017); SEQ ID NO:344. Additionally, in all WT PD-1 moieties, an unpaired cysteine was mutated to serine to remove the liability of an exposed reducing group (SEQ ID NO: 345). Some variants also contained a cleavage sequence for the tumor microenvironment (TME)-associated protease uPa (MSGRSANA SEQ ID NO: 10), to allow for the removal of part or all of the mask by exposure of the fusion protein to protease. A schematic of the construct design for a masked Fab as well as the intended mechanism of action is shown in Table 16. The final designs were bispecific Fab×scFv Fc molecules that contain a masked anti-CD3 Fab as well as an anti-Her2 scFv. A schematic is shown in Table 16 and the clones used are listed below in Table 16. Sequences are provided in Table 24.

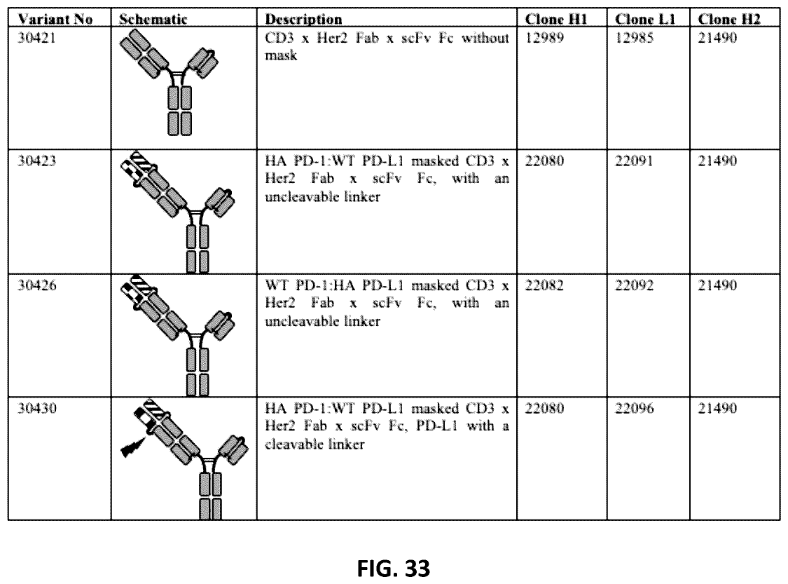

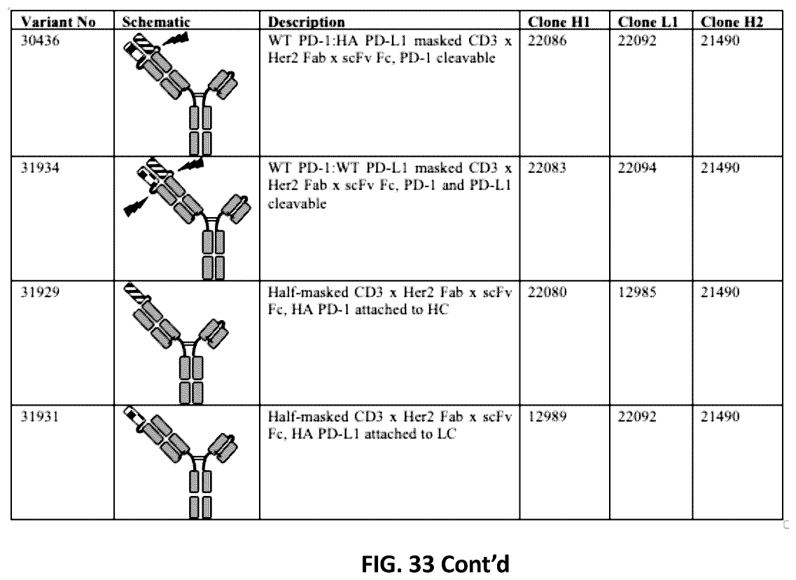

TABLE 16
Sequence composition of tested variants
Schematics are shown in FIG. 33

| Variant | | Clone | Clone | Clone |
|---|---|---|---|---|
| No | Description | H1 | L1 | H2 |
| 30421 | CD3 × Her2 Fab × scFv Fc without mask | 12989 | 12985 | 21490 |
| 30423 | HA PD-1: WT PD-L1 masked CD3 × Her2 Fab × | 22080 | 22091 | 21490 |
| | scFv Fc, with an uncleavable linker | | | |
| 30426 | WT PD-1: HA PD-L1 masked CD3 × Her2 Fab × | 22082 | 22092 | 21490 |
| | scFv Fc, with an uncleavable linker | | | |
| 30430 | HA PD-1: WT PD-L1 masked CD3 × Her2 Fab × | 22080 | 22096 | 21490 |
| | scFv Fc, PD-L1 with a cleavable linker | | | |
| 30436 | WT PD-1: HA PD-L1 masked CD3 × Her2 Fab × | 22086 | 22092 | 21490 |
| | scFv Fc, PD-1 cleavable | | | |
| 31934 | WT PD-1: WT PD-L1 masked CD3 × Her2 Fab × | 22083 | 22094 | 21490 |
| | scFv Fc, PD-1 and PD-L1 cleavable | | | |
| 31929 | Half-masked CD3 × Her2 Fab × scFv Fc, HA PD- | 22080 | 12985 | 21490 |
| | 1 attached to HC | | | |
| 31931 | Half-masked CD3 × Her2 Fab × scFv Fc, HA PD- | 12989 | 22092 | 21490 |
| | L1 attached to LC | | | |

Sequences of modified CD3×Her2 Fab×scFv variants were then ported into expression vectors and expressed and purified largely as described in Protocols 1, 2 and 6

Samples contained significant amounts of higher molecular weight species as determined by UPLC-SEC after protein A purification (not shown) and preparative SEC was used in order to obtain samples of high purity. Yields after preparative SEC ranged from 1.5-5 mg per variant. Sample purity and stability was assessed largely as described in Protocols

### Purity and Homogeneity Assessment of Masked Anti-CD3 Variants

Purified variants were assessed for purity and sample homogeneity by non-reducing/reducing Caliper UPLC-SEC as described below.

Methods

Following purification, purity of samples was assessed by non-reducing and reducing High Throughput Protein Express assay using Caliper LabChip® GXII (Perkin Elmer, Waltham, Mass.). Procedures were carried out according to HT Protein Express LabChip® User Guide version 2 with the following modifications. mAb samples, at either 2 ul or 5 ul (concentration range 5-2000 ng/ul), were added to separate wells in 96 well plates (BioRad, Hercules, Calif.) along with 7 ul of HT Protein Express Sample Buffer (Perkin Elmer #760328). The reducing buffer is prepared by adding 3.5 μL of DTT(1M) to 100 μL of HT Protein Express Sample Buffer. mAb samples were then denatured at 90° C. for 5 mins and 35 μl of water is added to each sample well. The LabChip® instrument was operated using the HT Protein Express Chip (Perkin Elmer #760499) and the HT Protein Express 200 assay setting (14 kDa-200 kDa).

UPLC-SEC was performed on an Agilent Technologies 1260 Infinity LC system using an Agilent Technologies AdvanceBio SEC 300A column at 25° C. Before injection, samples were centrifuged at 10000 g for 5 minutes, and 5 mL was injected into the column. Samples were run for 7 min at a flow rate of 1 mL/min in PBS, pH 7.4 and elution was monitored by UV absorbance at 190-400 nm. Chromatograms were extracted at 280 nm. Peak integration was performed using the OpenLAB CDS ChemStation software.

Results

UPLC-SEC traces of samples after preparative SEC purification of the variants 30421, 30423, 30430, and 30436 showed highly homogeneous samples that contained 89%-94% of correct species. The presence of a small peak at a low retention time compared to the main species indicated the presence of small amounts of high molecular weight species such as oligomers and aggregates in all samples.

Analysis of non-reducing Caliper showed a single predominant species and only bands corresponding to the intact chains of all variants were found in the reducing Caliper run. Notably, the masked heavy and light chains showed a significantly higher apparent molecular weight than what would be expected (110 kDa vs 63 kDa for the HC, 54 kDa vs 37 kDa for the LC). This was also reflected in the high apparent molecular weight of the non-reduced, disulfide bonded species (215 kDa vs 152 kDa). Glycosylation of both the PD1 and PD-L1 moieties in the designs is likely causing the increase in apparent molecular weight (Tan, S. et al. An unexpected N-terminal loop in PD-1 dominates binding by nivolumab. Nat Commun 8, 14369, doi:10.1038/ncomms14369 (2017), Li, C. W. et al. Glycosylation and stabilization of programmed death ligand-1 suppresses T-cell activity. Nat Commun 7, 12632, doi:10.1038/ncomms12632 (2016)).

### Stability Assessment of Masked Anti-CD3 Variants

Purified variants were assessed for thermal stability by differential scanning calorimetry (DSC) largely as described Protocol 11.

Results

The DSC thermogram of the unmodified CD3×Her2 Fab×scFv Fc variant (30421) showed transitions at 68 and 83° C. While the transition with a Tₘ of 68° C. likely corresponds to unresolved individual transitions for unfolding of the anti-CD3 Fab, anti-Her2 scFv and CH2 domain, the transition at Tₘ=83° C. likely corresponds to unfolding of the CH3 domain in the heavy chain. Thermograms of variants bearing a PD-1:PD-L1 mask (30430, 30436) also showed two transitions at similar temperatures and with similar thermogram traces to the unmasked variant. This indicates that the fused masking domains do not affect the Tₘ of the anti-CD3 Fab, and either unfold cooperatively with the Fab or uncooperatively but with a similar Tₘ to Fab, scFv and CH2.

### uPa Cleavage of Anti-CD3 Variants

In order to assess release of part of or all of the mask from the anti-CD3 Fab of the fusion proteins by cleavage of the introduced protease cleavage sites in the linkers, samples were treated with uPa in vitro. Reactions were monitored by reducing Caliper as follows.

Methods

For a preparative cleavage of the variants, 25-100 ug of purified sample was diluted to a final variant concentration of 0.2 mg/mL in PBS+0.05% Tween20 and Recombinant Human u-Plasminogen Activator (uPa)/Urokinase (R&D Systems #P00749) was added at a 1:50 protease: substrate ratio. After incubation at 37° C. for 24 h, sample fragments were analyzed in reducing Caliper and then frozen and stored at −80° C. until further use.

Results

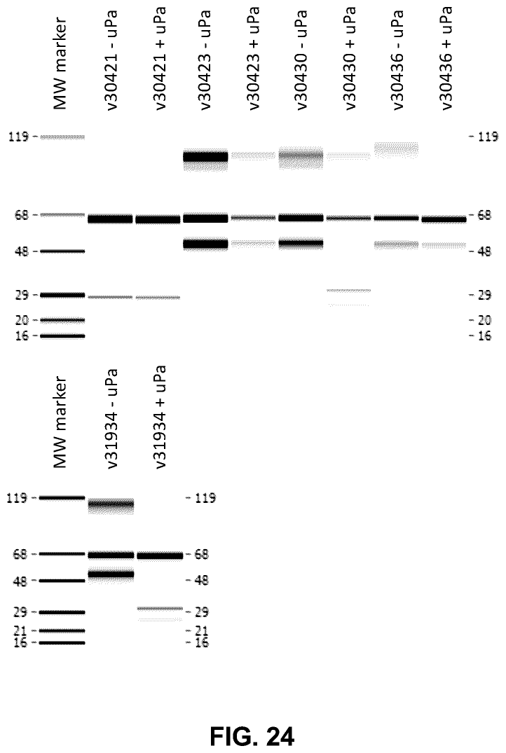

Analysis of reducing Caliper profiles of the masked variants before and after uPa treatment revealed that under the investigated conditions, part or all of the mask was removed from the Fab effectively by cleavage at the introduced cleavage sites (FIG. 24). For successfully cleaved variants (30430, 30436, 31934), bands representing fragments of masked heavy and/or light chain disappeared completely upon cleavage while fragments of un-masked heavy and/or light chain appear. While a broad band of low intensity corresponding to a fragment of free PD-1 can be observed for variant 30430, this was not the case for the released PD-L1 in variant 30436. Small size and size heterogeneity due to glycosylation (Tan, S. et al. An unexpected N-terminal loop in PD-1 dominates binding by nivolumab. Nat Commun 8, 14369, doi:10.1038/ncomms14369 (2017), Li, C. W. et al. Glycosylation and stabilization of programmed death ligand-1 suppresses T-cell activity. Nat Commun 7, 12632, doi:10.1038/ncomms12632 (2016)) likely rendered the free PD-1 and PD-L1 fragments barely detectable and undetectable, respectively. In variants that do not contain the cleavage sequence (30421, 30423), no cleavage was observed.

### Masking/Unmasking of CD3-Binding

Uncleaved and cleaved samples of anti-CD3 variants were tested for binding to CD3 expressing Jurkat cells by ELISA as follows.

Methods

Human Jurkat cells (Fujisaki Cell Center, Japan) were maintained in RPMI-1640 medium supplemented with 2 mM L-glutamine and 10% of heat-inactivated fetal bovine serum (FBS) with 1× Penicillin/Streptomycin, in a humidified+5% CO2 incubator at 37° C.

Samples of modified CD3×Her2 variants were diluted 2× in blocking buffer, followed by seven three-fold serial dilutions in blocking buffer for a total of eight concentration points. Blocking buffer alone was added to control wells to measure background signal on cells (negative/blank control).

All incubations were performed at 4° C. On the day of the assay, exponentially growing cells were centrifuged and seeded in a 96-well filter plate (MilliporeSigma, Burlington, Mass., USA) in a 1:1 mixture of complete culture medium and blocking buffer. Equal volumes of 2×variants or controls were added to cells and incubated for 1 hour. The plate was then washed 4 times using vacuum filtration. An HRP-conjugated anti-human IgG Fc gamma specific secondary antibody (Jackson ImmunoResearch, West Grove, Pa., USA) was added to the wells and further incubated for 1 h. Plates were washed 7 times by vacuum filtration followed by the addition of TMB substrate (Thermo Scientific, Waltham Mass., USA) at room temperature. The reaction was stopped by adding 0.5 volume of 1 M sulfuric acid and the supernatant was transferred by filtration into a clear 96-well plate (Corning, Corning, N.Y., USA). Absorbance at 450 nm was read on a Spectramax 340PC plate reader with path-check correction.

Binding curves of blank-subtracted OD450 versus linear or log antibody concentration were fitted with GraphPad Prism 8 (GraphPad Software, La Jolla, Calif., USA). A one-site specific, four-parameter nonlinear regression curve fitting model with Hill slope was employed in order to determine Bmax and apparent Kd values for each test article.

Results

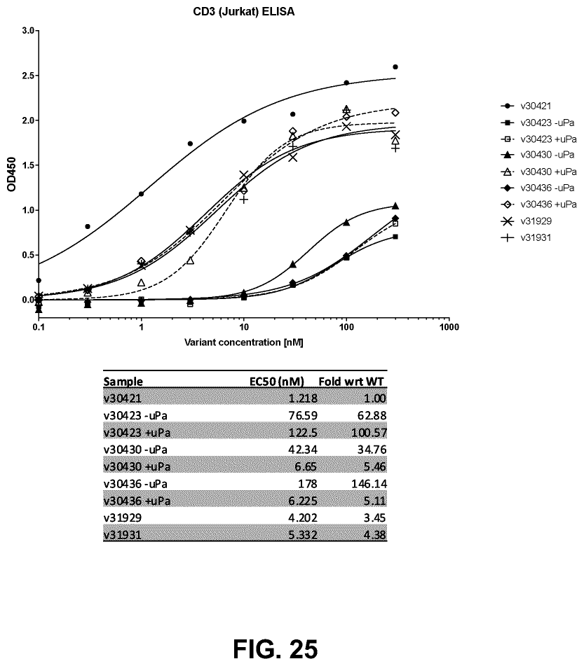

As can be seen in FIG. 25, variants containing a full PD1:PD-L1 based mask appended to the CD3 Fab (30423, 30430, 30436) showed 40-180 fold reduced binding compared to the unmasked control (30421). Upon treatment with uPa, CD3 binding of the cleavable variants 30430 and 30436 was partially restored (within 6-7 fold of the unmasked control). This partial recovery might be caused by a steric hinderance of epitope binding by the portion of the mask that is left on the mask after cleavage. Concomitantly, controls that only had PD-1 or PD-L1 appended to either heavy or light chain, respectively (31929, 31931), showed a similar reduction (4-5 fold) in binding compared to the unmasked control as the uPa-cleaved samples of the fully masked variants.

### T-Cell Dependent Cellular Cytotoxicity of Masked and Unmasked Variants

The functional impact of the PD-1:PD-L1 based mask on the ability of the CD3×Her2 Fab×scFv Fc variants to engage and activate T-cells for the killing of Her2-bearing cells was assessed in a T-cell dependent cellular cytotoxicity (TDCC) assay as follows.

Methods

Coculture Assay

JIMT-1 (Leibniz Institute, Braunschweig, Germany), that are Her2 positive and express ˜500 000 receptors per cell, were thawed and cultured in growth medium prior to experiment set-ups. The growth medium consisted of McCoy's 5A and DMEM medium (A1049101, ATCC modification) (Thermo Fisher Scientific, Waltham, Mass.) supplemented with 10% Fetal Bovine Serum (Thermo Fisher Scientific, Waltham, Mass.) respectively. The cells were maintained horizontally in T-75 flasks (VWR, Radnor, Pa.) in an incubator at 37° C. with 5% carbon dioxide. On the day of the experiment, the variants were titrated in triplicate at 1:3 dilution directly in a 384-well cell culture treated optical bottom plates (ThermoFisher Scientific, Waltham, Mass.) from 5 nM to 0.08 pM. JIMT-1 cells were harvested using TrypLE (ThermoFisher Scientific, Waltham, Mass.) washed in media, and counted. A vial of primary human pan-T cells (BioIVT, Westbury, N.Y.), was thawed in a 37° C. water bath, washed in media, and counted. Pan T cell suspension was mixed with JIMT-1 cells at 5:1 effector to target ratio, washed and resuspended at 0.55E6 cell/ml. 20 uL of the mixed cell suspension was added to the plate containing the titrated variants.

The plates were incubated for 48 hr in an incubator at 37° C. with 5% carbon dioxide. The samples were then subjected to a high-content cytotoxicity assessment.

High Content Cytotoxicity Analysis

For visualization of nuclei and assessment of viability, cells were stained with Hoechst33342. 10 uL of Hoechst33342 was diluted 1:1000 in media, added to the cells after the 48 h period and incubated for a further 1 hr at 37° C. Then, the plate was subjected to high content image analysis on CellInsight CX-5 (ThermoFisher Scientific, Waltham, Mass.) in order to distinguish and quantify viable and dead tumor cells as well as effector cells. The plate was scanned on the CellInsight CX5 high content instrument using the SpotAnalysis.V4 Bioapplication with the following settings: Objective: 10×, Channel 1— 386 nm: Hoechst (Fixed exposure time 0.008 ms with a Gain of 2).

Results

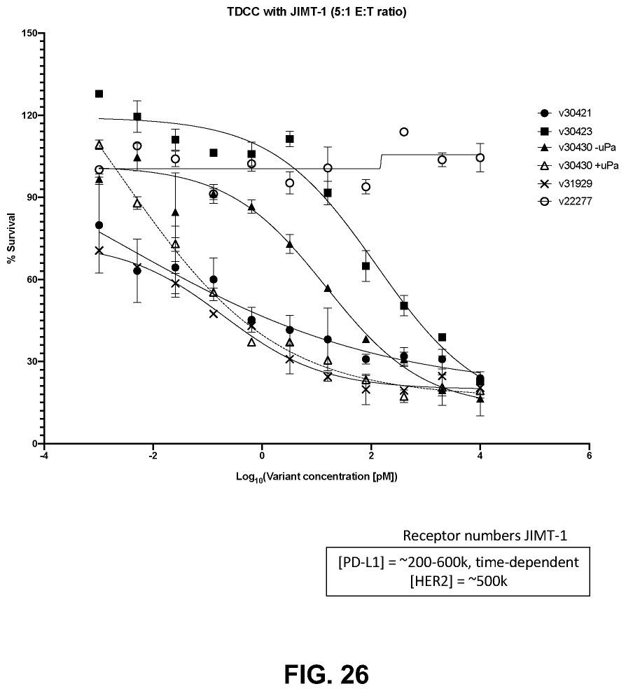

The masking effects seen above for the CD3×Her2 Fab×scFv Fc variants in binding to CD3 were recapitulated when the same samples were interrogated for function in a TDCC assay with Her2 expressing JIMT-1 cells (FIG. 26). While the unmasked variant (30421) showed robust tumor cell killing at low variant concentrations, the potency of a masked, uncleavable variant (30423) was decreased by 1000×. A fully masked variant with a cleavable PD-L1 moiety on the light chain (30430) was also reduced in potency before uPa treatment, by ˜100×. This discrepancy in masking between uncleavable and cleavable variants was seen above for CD3 binding as well and is likely due to the increased flexibility in one of the linkers introduced by the uPa cleavage site which added 8 amino acids to the length of the linker. After cleavage of the mask by uPa, the potency of 30430 returned to that of an unmasked (30421) variant. A control variant with only the PD-1 moiety of the mask attached (31929) showed similar potency to 30421 and uPa-treated 30430. An irrelevant anti Respiratory Syncytial Virus (RSV) antibody (22277) showed no activation of T cells for tumor cell killing.

The above experiments further confirm that the MSGRSANA (SEQ ID NO:10) uPa cleavage site can be transferred into a variety of recombinant proteins of different formats, having different masks and be effectively cleaved to unmask a desired protein.

### Example 17: Testing the Effect of Scfv Mask VH-VL Linker Cleavage on IL12 Binding

As described in Example 5, some scFv-masked IL12 HetFc Fusion Proteins were designed with an additional protease cleavage sequence within the linker between the VH and VL domains of the scFv mask, which was hypothesized to aid in recovery of IL12 activity by destabilizing the scFv upon protease cleavage and accelerating its release. To test this hypothesis, Fc-scFv fusions were produced with or without a protease cleavage sequence between the scFv VH and VL, digested with Matriptase, and tested for IL12 binding by SPR.

### Methods

Fc-scFv fusions were designed in the same format as masked IL12 HetFc fusion proteins but without IL12 moieties, i.e. as HetFc heterodimers with a scFv linked to the C-terminus of one HetFc chain through a protease-cleavable linker, where the scFv optionally contains a second protease cleavage sequence within the linker between the VH and VL. Variants are listed in Table 17. Variants were produced as described in Example 7, digested with Matriptase as described in Example 8, and tested for IL12 binding by SPR as described in Example 3.

TABLE 17
Briakinumab HetFc-scFv fusion variants

| Variant ID | HetFc 1 clone ID | HetFc 2 clone ID |
|---|---|---|
| v32909 (cleavable VH-VL linker) | CL_#22735 | CL_#12155 |
| v32910 (non-cleavable VH-VL | CL_#23571 | CL_#12155 |
| linker) | | |

### Results:

Both variants displayed IL12 binding kinetics similar to those determined in Example 3 for Briakinumab Fab and scFv controls, both with and without cleavage by Matriptase, indicating that cleavage of neither the Fc-scFv linker nor the VH-VL linker is detrimental to IL12 binding (Table 18; note that ka are near instrument detection limit).

TABLE 18
SPR binding to immobilized IL12.

| | Kinetics pre-digest | | | Kinetics post-digest | | |
|---|---|---|---|---|---|---|
| Variant ID | ka (1/Ms) | kd (1/s) | KD (M) | ka (1/Ms) | kd (1/s) | KD (M) |
| v32909 | 2.25E+06 | 4.48E−05 | 1.98E−11 | 4.19E+07 | 6.48E−05 | 1.47E−12 |
| v32910 | 2.10E+06 | 4.09E−05 | 1.94E−11 | 8.65E+06 | 4.68E−05 | 5.37E−12 |

### Example 18: Design, Production, and Testing of Reduced-Potency IL12 HetFc Fusion Proteins

To reduce the IL12 activity of masked and non-masked IL12 HetFc fusion proteins for better overall tolerability, mutations were made to the IL12 p35 or p40 domains to reduce binding to the receptors IL12Rβ1 and IL12Rβ2.

### Methods:

To design IL12 HetFc fusion proteins with reduced binding to IL12Rβ1 and IL12Rβ2, amino acids within the p35 and p40 domains of IL12 that contribute to IL12 stability or that potentially interact directly with IL12Rβ1 and IL12Rβ2 were identified based on analyses considering structural contacts between p35 and p40, sequence conservation among IL12 orthologues, expected structural homology of IL12-IL12Rβ2 with the IL23-IL23R complex (pdb 5mzv), epitope comparisons of known IL12Rβ1 and/or IL12Rβ2 blocking antibodies (e.g. Briakinumab, pdb 5njd; Ustekinumab, pdb 3hmx; antibodies 22E11, 124C4, and 37D5, pdb 5mzv), and regions of excess surface charge. The identified amino acids or groups thereof were then mutated to alter their size, polarity, and/or charge. Non-masked and masked IL12 HetFc fusion proteins with the selected mutation(s) were constructed as described in examples 1 and 5. Mutations made to IL12 and corresponding clone and variant IDs for IL12 HetFc fusion proteins are listed in Table 19.

Proteins were produced and characterized biophysically as described in Example 7 and tested for CD8+ T cell activity to assess the reduction in potency of the non-masked and masked molecules with mutated IL12 domains relative to corresponding controls with wild-type IL12 as described in Example 12.

TABLE 19
IL12 p35 and p40 mutations designed to reduce IL12 activity,
and corresponding masked and non-masked reduced-potency
IL12 HetFc fusion protein clone and variant IDs.

| | | | | Non-masked |
|---|---|---|---|---|
| | | | Masked | reduced- |
| | | | reduced-potency | potency |
| | | | IL12 HetFc | IL12 HetFc |
| p35 | p40 | HetFc1 | fusion protein | fusion protein |
| mutations | mutations | clone ID^{a} | Variant ID^{b} | Variant ID^{c} |
| | S175V | CL_#24831 | 33501 | 33489 |
| | A179T | | | |
| | S183T | | | |
| | S294N | | | |
| L68A | | CL_#24832 | 33502 | 33490 |
| R181A | | CL_#24833 | 33503 | 33491 |
| V185A | | CL_#24834 | 33504 | 33492 |
| E38R | | CL_#24835 | 33505 | 33493 |
| K128E | | | | |
| K168E | | | | |
| P41S | | CL_#24836 | 33506 | 33494 |
| I171Q | | | | |
| I175S | | | | |
| F39S | | CL_#24837 | 33507 | 33495 |
| Y40S | | | 35425^{d} | |
| Y167S | | | 35427^{d} | |
| | | | 35437^{f} | |
| T43A | | CL_#24838 | 33508 | 33496 |
| E45R | | | | |
| I47S | | | | |
| D48R | | | | |
| T43A | | CL_#24839 | 33509 | 33497 |
| E45R | | | | |
| E46K | | | | |
| I47S | | | | |
| D48R | | | | |
| E50K | | | | |
| | D41S | CL_#24840 | 33510 | 33498 |
| | E45R | | 36190^{e} | |
| | K58S | | 36193^{f} | |
| | E59S | | | |
| | K195D | | | |
| | K99S | CL_#24841 | 33511 | 33499 |
| | E100S | | | |
| | R159S | | | |
| | E187S | CL_#24842 | 33512 | 33500 |
| | T202S | | | |
| | S204R | | | |

^{a}HetFc2 clone ID is CL_# 12153 for all Non-masked IL12 HetFc variants and CL_#22735 for all Masked IL12 HetFc variants unless noted otherwise
^{b}All Masked IL12 HetFc fusion protein variants are derived from v31277 with the addition of the specified p35 or p40 mutations unless noted otherwise
^{c}All Non-masked IL12 HetFc fusion protein variants are derived from v30806 with the addition of the specified p35 or p40 mutations
^{d}Variants 35425 and 35427 are derived from variants 32862 and 35426, respectively, where variant 35425 uses HetFc2 clone CL_#24224 (similar to CL_#22735 but lacking the second protease cleavage sequence within the scFv VH-VL linker) and variant 35427 uses HetFc2 clone CL_#26498 (same as CL_#24224 but with the scFv H_F27V mutation)
^{e}Variant 36190 is dervied from variant 32862, using HetFc2 clone CL_#24224 (similar to CL_#22735 but lacking the second protease cleavage sequence within the scFv VH-VL linker)
^{f}Variants 35437 and 36193 are derived from variants 35425 and 36190, respectively, but use HetFc2 clone CL_#26503 (similar to CL_#24224 but with an alternate non-cleavable linker between the HetFc and scFv VH domains)

### Results:

Yields and UPLC-SEC monomer purity after Protein-A purification were between 43-75 mg/L and 46-73% for non-masked variants with mutated p35 or p40 domains, compared to 64 mg/L and 79% for a non-masked control variant with wild-type IL12, and were between 30-62 mg/L and 66-80% for masked variants with mutated p35 or p40 domains (excluding variants 35425, 35427, 35437, 36190, and 36193, which were not expressed in this group), compared to 47 mg/L and 76% for a masked control variant with wild-type IL12. All samples were purified to >95% monomer by Prep-SEC, except for v33500 that was to 93%. CD8+ T cell IFNγ release after incubation in the presence of the masked and non-masked IL12 HetFc fusion protein variants designed for reduced potency is summarized in

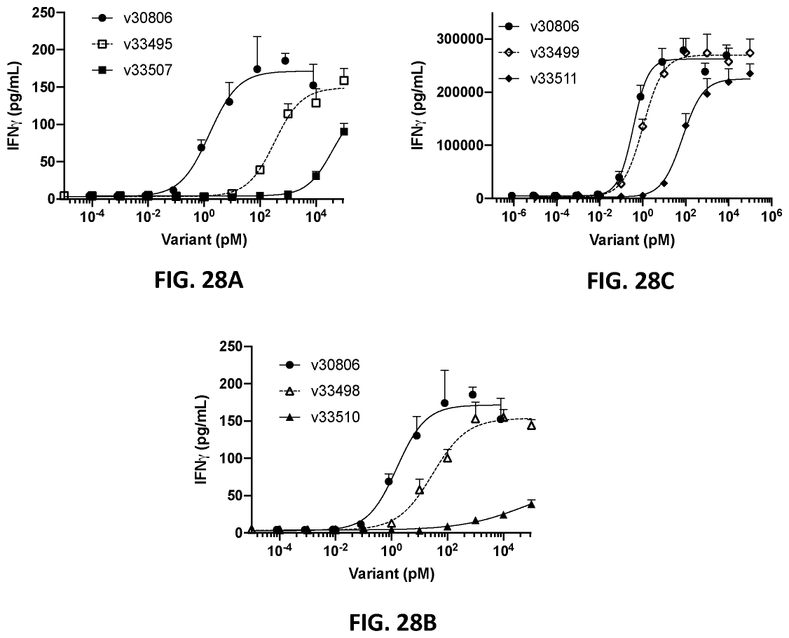

FIGS. 28A-28C and Table 20. The majority of non-masked variants showed a reduction in potency of no more than 5-fold compared to wild-type IL12 control v30806. Three variants, v33495, v33498, and v33499, showed reduction in potency as non-masked constructs, but upon masking were markedly reduced in potency from wild-type IL12 control 30806. The potencies of the non-masked variants v33495, v33498, and v33499 were 395-fold, 17-fold, and 3-fold lower than v30806, respectively, and the potencies of the corresponding masked variants v33507, v33510, and v33511 were 51996-fold, 5562-fold, and 195-fold lower than v30806, respectively. When comparing non-masked and masked variants with the same IL12 mutations, there was a 132-fold potency reduction between v33495 and v33507, 329-fold between v33498 and v33510, and 67-fold between v33499 and v33511 (FIG. 28). Compared to the 69-fold potency difference between v31277 and v30806 (corresponding masked and non-masked IL12 HetFc fusion variants with wild-type IL12; Example 12) it is evident that certain attenuated IL12 designs synergize with the scFv mask to generate even larger masking windows, which may result from differences in how well each design prevents residual binding or competition of IL12Rβ1 and/or IL12Rβ2 in the presence vs. absence of mask.

TABLE 20
Fold change in IFNγ production by CD8T Cells
when treated with reduced-potency IL12 variants

| Non-masked | Fold Change in Potency compared to parental non- |
|---|---|
| reduced-potency | masked WT IL12 variant v30806 |
| IL12 variant | (Variant EC50/Parental variant EC50) |
| 33489 | 3.6× |
| 33490 | 2.8× |
| 33491 | 3.6× |
| 33492 | 4.4× |
| 33493 | 1.0× |
| 33494 | 1.0× |
| 33495 | 395× |
| 33496 | 0.42× |
| 33497 | 0.42× |
| 33498 | 17× |
| 33499 | 3.3× |
| 33500 | 3.7× |
| Masked | Fold Change in Potency compared to parental non- |
| reduced-potency | masked WT IL12 variant v30806 |
| IL12 variant | (Variant EC50/Parental Variant EC50) |
| 33507 | 51996× |
| 33510 | 5562× |
| 33511 | 195× |
| | Fold Change in Potency compared to corresponding |
| | non-masked reduced-potency IL12 variants^{a} |
| | (Masked Variant EC50/Non-masked Variant EC50) |
| 33507 | 132× |
| 33510 | 329× |
| 33511 | 67× |

^{a}Corresponding non-masked reduced-potency IL12 variants for v33507, v33510, and v33511 are v33495, v33498, and v33499, respectively.

### Example 19: Design and Testing of Modified Linkers for Matriptase Cleavage Rate

It may be desirable to adjust the overall susceptibility to cleavage of protease-cleavable linkers within masked IL12 HetFc fusion proteins to balance cleavage rates in the tumour microenvironment with potential off-tumour cleavage. This example describes the design and testing of masked IL12 HetFc fusion proteins with shortened protease-cleavable linkers to modulate protease accessibility.

### Methods:

Masked IL12 HetFc fusion protein variants with shortened protease-cleavable linkers were designed based on variant v31277, where linker sequences on either or both sides of the protease cleavage motif were successively shortened. Variants are described in Table 21.

TABLE 21
Masked IL12 HetFc fusion protein variants
with shortened protease-cleavable linkers

| Variant ID | HetFc-mask linker sequence | HetFc1 clone ^{a} |
|---|---|---|
| v31277 | (G4S)2-MSGRSANA-(G4S)2 | CL_#22735 |
| v32857 | (G4S)2-MSGRSANA-G4S | CL_#24219 |
| v32945 | G4S-MSGRSANA-(G4S)2 | CL_#24308 |
| v32859 | G4S-MSGRSANA-G4S | CL_#24221 |
| v32860 | GGS-MSGRSANA-GGS | CL_#24222 |

^{a} All variants utilize HetFc2 clone CL_#22279

Proteins were produced and characterized biophysically as described in Example 7. Susceptibility of modified linkers within masked IL12 HetFc fusion protein variants to protease cleavage was determined by a time-course Matriptase digestion, performed as described in Example 8, with aliquots removed at various time points and assessed by reducing CE-SDS. Variants were also tested for CD8+ T cell activity as described in Example 12 to assess if shortening the HetFc-mask linker had an impact on the efficiency of masking.

### Results:

Yields and UPLC-SEC monomer purity after Protein-A purification were between 45-69 mg/L and 55-58% for masked variants with shortened HetFc-mask protease cleavable linkers, compared to 45 mg/L and 66% for parental variant v31277. All samples were purified to >97% monomer by Prep-SEC.

The time course Matriptase digest revealed that the protease-cleavable HetFc-mask linker of the parental variant v31277 was fully cleaved after 4 hours, and the time for complete cleavage increased with shortened HetFc-mask linker lengths up to 24 hours for variant v32860 (Table 22).

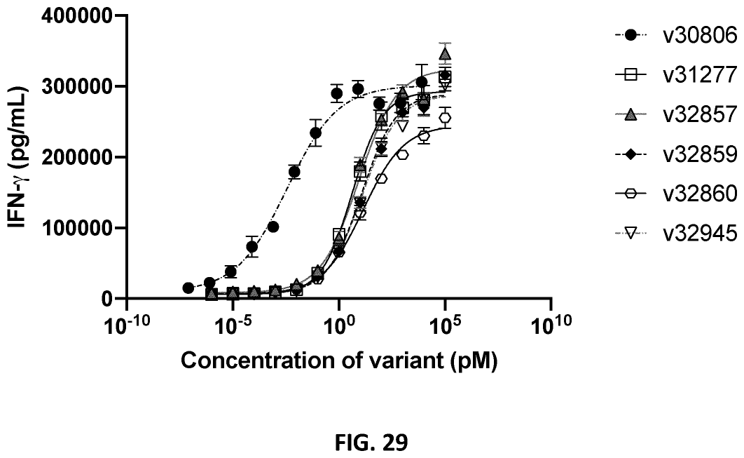

CD8+ T cell IFNγ release after incubation in the presence of the masked IL12 HetFc fusion protein variants designed with shortened cleavable linkers is summarized in FIG. 29. All variants had comparable potency to v31277, with the exception of v32860, which showed an approximate 2-fold reduction in potency compared to 31277 across 3 experiments.

TABLE 22
Time course Matriptase digestion of masked IL12 HetFc fusion
protein variants with modified protease-cleavable linkers

| | % cleavage at time points^{a} | | | | | | |
|---|---|---|---|---|---|---|---|
| Variant | 0 hr | 1 hr | 2 hr | 4 hr | 6 hr | 8 hr | 24 hr |
| v31277 | 0 | 62 | 84 | 100 | 100 | 100 | 100 |
| v32857 | 0 | 34 | 60 | 100 | 100 | 100 | 100 |
| v32945 | 0 | 34 | 58 | 82 | 100 | 100 | 100 |
| v32859 | 0 | 24 | 48 | 75 | 100 | 100 | 100 |
| v32860 | 0 | 18 | 38 | 61 | 73 | 79 | 100 |

^{a}% cleavage calculated by dividing the total intensity of bands corresponding to cleaved HetFc-mask species by that of non-cleaved HetFc-mask species using reducing CE-SDS

### Example 20: Indication Selection for IL-12Fc Protease-Cleavable Fusion Proteins

Increased expression of proteases has been reported in multiple cultured tumor cell lines, in vivo xenografts and human tumor tissue. It is hypothesized that tumor types with increased protease expression and or activity could be suitable indications for clinical application of IL-12Fc fusions containing protease-cleavable masks. This may be especially true in tumor types that are also highly infiltrated with immune cells expected to be stimulated by IL-12. This example describes the identification of human tumor tissues with immune cell infiltration, high protease expression and or activity, and validation of IL-12Fc fusion protein variant cleavage in human tumor material.

### Methods:

In order to identify cancer types that demonstrate high infiltration of immune cells as well as high mRNA expression of uPA or Matriptase proteases, TCGA (https://www.cancer.gov/tcga) and GTEx (Carithers, L. J. et al. A novel approach to high-quality postmortem tissue procurement: the GTEx project. Biopreserv. Biobank. 13, 311-319 (2015)) datasets were extensively investigated. First, human tumor types that have high infiltration of immune cell subsets, including macrophages, dendritic cells, NK cells and T cells were identified by CIBERSORT based on analyzing TCGA mRNA-seq data (Newman, A. M., et al. Robust enumeration of cell subsets from tissue expression profiles. Nat. Methods 12, 453-457 (2015); Thorsson, V. et al. The immune landscape of cancer. Immunity 48, 812-830 (2018)). CIBERSORT estimates the relative fraction of 22 immune cell types within a bulk tumor RNA-seq sample using a deconvolution-based approach and sets of pre-defined immune cell reference profiles. Hence, for each TCGA sample, the relative immune cell infiltration fraction was estimated by CIBERSORT (Thorsson et al, 2018) and a total immune fraction was estimated by summing up the predicted fractions for the following cell types: Dendritic Cells+NK+Macrophages (excluding M2)+Monocytes+Neutrophils+Eosinophils+CD4 T-Cells+CD8 T Cells. A median infiltration fraction for each cancer type was then computed by taking a median of infiltration fractions from all samples within that cancer type. Next, human tumor types or normal tissues that demonstrate high mRNA expression of uPA and matriptase were identified by analysis of TCGA, or GTEx mRNA sequencing data sets, respectively. The mRNA expression levels were reported as TPM values (Transcript Per Million). Median values of protease mRNA expression levels were generated for each cancer type. Cancer types with high median mRNA expression of proteases as well as high median immune cell infiltration were identified for further investigation.

To test the potential of masked IL12 HetFc fusion protein activation in predicted protease high expressing human tumors, protease-cleavable and non-cleavable masked IL12 HetFc fusion proteins were assessed by LC-MS for cleavage after incubation in human tumor tissue material. Lysates were generated from homogenized human pancreatic tumor tissue and cell supernatant removed from BxPC3 pancreatic tumor cells in monolayer cell culture. Variants were incubated in lysate or supernatant for 72 hours at 37° C., deglycosylated for 16 hours at 37° C. and purified used anti-human IgGFc followed by reduction and analysis by LC-MS.

### Results:

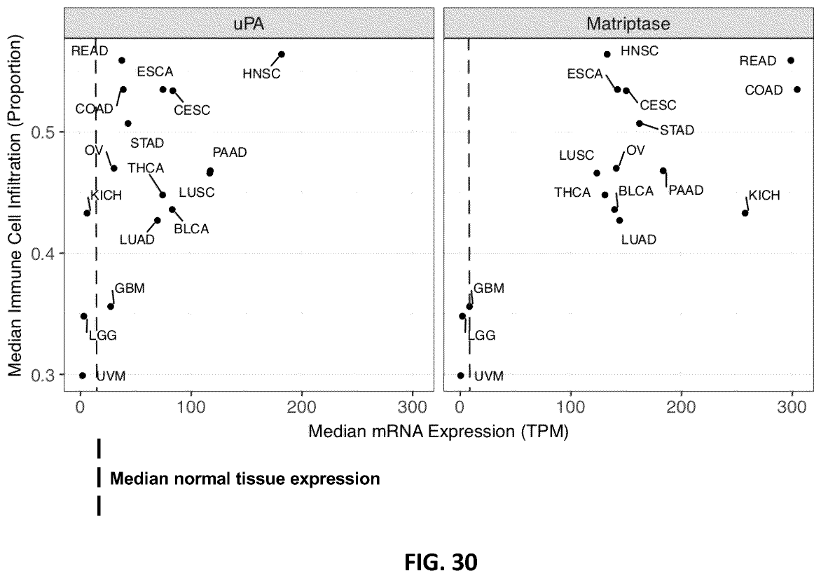

The analysis of median tumor immune infiltration fraction and protease mRNA expression indicated that several tumor types, including head and neck (HNSC), pancreatic (PAAD), thymic (THCA), lung (LUSC, LUAD), esophageal (ESCA), cervical (CESC), bladder (BLCA), rectal (READ) and colon (COAD) showed a high degree of both immune cell infiltration and both uPA and matriptase mRNA expression. For these tumor types, median protease expression was above median normal tissue expression (computed from GTEx). Although identified as having immune cell infiltration, chromophobe renal cell carcinoma showed above normal tissue expression of only matriptase but not uPA (FIG. 30).

After incubation of cleavable variant v31277 in human pancreatic BxPC3 tumor cell supernatant, analysis of mass by LC-MS indicated the presence of species corresponding to cleavage within the designed protease cleavage motifs in the HetFc-mask chain, compared to only intact HetFc-mask observed after incubation in PBS. Similar results were observed for variant incubated in pancreatic tumor lysate. Only intact HetFc-mask was observed for the non-cleavable variant v32041 incubated in PBS or tumor cell supernatant or lysate. These results indicate that masked IL12 HetFc fusions are susceptible to cleavage at the designed protease-cleavable linkers by proteases in human tumor tissue material.

### Example 21: Masked Non-Cleavable IL12-Fc Variants have Greater Tolerability Compared to IL12-Fc in Stem Cell Humanized Mice

### Methods:

In order to assess the ability of an engineered mask to reduce the potency of IL12-Fc in vivo, variants were tested in a humanized mouse model of toxicity. Immunodeficient NOD-scid-Gamma (NSG) mice were engrafted with human CD34+ hematopoietic stem cells to reconstitute components of a human immune system within the mouse peripheral blood and lymphoid tissues. CD34+ stem cell engraftment in immunocompromised mice provides a stable and functional humanized immune system to assess T-cell responses to IL12-Fc.

Approximately 18 weeks after CD34+ engraftment, 10 mice each were administered two injections of either a vehicle control (v33936, 0 mg/kg), an unmasked IL12-Fc variant (v30806, 1 mg/kg), or masked non-cleavable IL12-Fc variant (v32041, 1.25 mg/kg) at matched molar doses. Mice were monitored for overall health and body weight after test article administration over a period of 60 days, and peripheral blood was analyzed on Day 20 for overall human cell engraftment and cell counts of specific linage populations. Serum was isolated from peripheral blood collected at all time points and frozen at −80° C. for subsequent pharmacokinetic analysis of variants. Presence of IL12 variants was assessed using an anti-human IL12 p35 antibody capture and anti-human Fc gamma detection sandwich MSD assay.

### Results:

Humanized mice dosed with vehicle remained healthy without any loss of survival to study day 60. Mice receiving unmasked IL12-Fc experienced the highest level of toxicity with a median survival of 33 days. The masked, non-cleavable variant exhibited a delayed onset of body weight loss and increased survival compared to the unmasked variant, with a median survival of 47 days.

Peripheral blood was collected and analyzed for the presence and frequency of human CD3+ T-cells as a readout of effector response to IL12 stimulation after test article administration. A baseline peripheral blood collection prior to the first variant injection indicated an average of 53.8+/−25.6 human CD3+ T-cells/uL of blood (represented as dashed and dotted lines with shading). Mice receiving injections of the unmasked IL12-Fc variant (v30806) exhibited a significant increase in the number of circulating CD3+ T-cells compared to mice that received the vehicle control alone (v33936) on study day 20. Meanwhile, mice receiving injections of the masked, non-cleavable IL12-Fc variant did not exhibit a significant increase in circulating CD3+ cell numbers on study day 20, indicating a reduction in potency of the test article. Incorporation of a mask onto the IL12-Fc resulted in a reduced expansion of human CD3+ cells in vivo and increased survival at molar matched dose in CD34+ humanized mice.

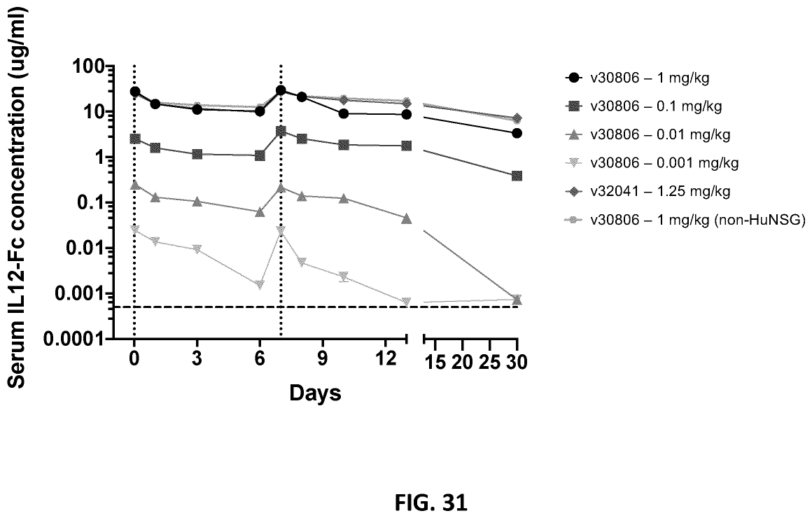

Serum PK analysis showed that non-masked IL12-Fc (v30806, 1 mg/kg) and masked IL12-Fc (v32041, 1.25 mg/kg) at matched molar doses displayed reasonable exposure over the 13 days of serum sampling (FIG. 31). Variants were still detectable in serum at an extended timepoint of 23 days post second dose (Day 30), indicating good in-vivo stability. Masked IL12-Fc (v32041, 1.25 mg/kg) had PK comparable to the non-cell engrafted NSG mice dosed with the molar equivalent non-masked drug (non-HuNSG, v30806, 1 mg/kg). Target mediated drug disposition (TMDD) was observed at lower doses of the non-masked IL12-Fc resulting in faster clearance, attributed to the expansion of CD3+ cells. No CD34+ donor dependent effect on PK was observed.

This indicates that masking IL12-Fc potency is functionally achievable, and the correct combination of masking and attenuation could yield a systemically tolerated and activatable IL12-Fc molecule.

TABLE 23
Clone descriptions

| AA SEQ | | |
|---|---|---|
| ID NO: | Clone ID | Domain structure^{abc} |
| 20 | CL_#12153 | HetFc |
| 21 | CL_#12155 | HetFc |
| 22 | CL_#17871 | p40 |
| 23 | CL_#17872 | p35 |
| 24 | CL_#17875 | HetFc-p35 |
| 25 | CL_#17876 | HetFc-GSADGG-p40-(G4S)3-p35 |
| 26 | CL_#17877 | p35-(G4S)2-HetFc |
| 27 | CL_#17879 | p40-(G4S)2-HetFc |
| 28 | CL_#17880 | HetFc-(G4S)2-p40 |
| 29 | CL_#17881 | HetFc-p35 |
| 30 | CL_#17906 | HetFc-p19 |
| 31 | CL_#17907 | p19-(G4S)2-HetFc |
| 32 | CL_#17908 | p19 |
| 33 | CL_#17942 | HetFc-(G4S)2-p40 |
| 34 | CL_#17945 | HetFc-GSADGG-p40-(G4S)4-p19 |
| 35 | CL_#18939 | Briak_{VH}-CH1-HetFc |
| 36 | CL_#18940 | Briak_{VL}-Cλ |
| 37 | CL_#18942 | Briak_{VH}-(G4S)3-Briak_{VL}-HetFc |
| 38 | CL_#18943 | Briak_{VL}-(G4S)3-Briak_{VH}-HetFc |
| 39 | CL_#18953 | HetFc-(G4S)-LSGRSDNH-(G4S)4-IL12Rβ2₂₄₋₃₂₁ |
| 40 | CL_#18954 | IL12Rβ2₂₄₋₃₂₁-(G4S)2-LSGRSDNH-(G4S)-HetFc |
| 41 | CL_#18956 | IL12Rβ2₂₄₋₁₂₄-(G4S)2-LSGRSDNH-(G4S)-p35-(G4S)2-HetFc |
| 42 | CL_#18957 | HetFc-GSADGG-p40-(G4S)3-p35-(G4S)-LSGRSDNH-(G4S)2- |
| | | IL12Rβ2₂₄₋₁₂₄ |
| 43 | CL_#21415 | p40-(G4S)2-MSGRSANA-(G4S)3-Briak_{VL}-(G4S)3-Briak_{VH} |
| 44 | CL_#21416 | HetFc-p35-(G4S)2-MSGRSANA-(G4S)3-Briak_{VL}-(G4S)3- |
| | | Briak_{VH} |
| 45 | CL_#21417 | HetFc-(G4S)2-MSGRSANA-(G4S)2-Briak_{VL}-(G4S)3-Briak_{VH} |
| 46 | CL_#21418 | Briak_{VH}-(G4S)3-Briak_{VL}-(G4S)3-MSGRSANA-(G4S)2-p40- |
| | | (G4S)2-HetFc |
| 47 | CL_#21419 | p35-(G4S)2-MSGRSANA-(G4S)3-Briak_{VL}-(G4S)3-Briak_{VH} |
| 48 | CL_#21421 | HetFc-(G4S)2-p40-(G4S)2-MSGRSANA-(G4S)3-Briak_{VL}- |
| | | (G4S)3-Briak_{VH} |
| 49 | CL_#21423 | HetFc-GSADGG-p40-(G4S)3-p35-(G4S)3-MSGRSANA- |
| | | (G4S)4-Briak_{VL}-(G4S)3-Briak_{VH} |
| 50 | CL_#21446 | Briak_{VH}-(G4S)3-Briak_{VL}-(G4S)2-MSGRSANA-(G4S)2-p40 |
| 51 | CL_#21447 | Briak_{VH}-(G4S)3-Briak_{VL}-(G4S)4-MSGRSANA-(G4S)3-p35 |
| 52 | CL_#21451 | Briak_{VH}-(G4S)3-Briak_{VL}-(G4S)4-MSGRSANA-(G4S)3-HetFc |
| 53 | CL_#21452 | Briak_{VH}-(G4S)3-Briak_{VL}-(G4S)3-MSGRSANA-(G4S)4-p35- |
| | | (G4S)2-HetFc |
| 54 | CL_#22203 | Briak_{VH}(Y32A)-(G4S)3-Briak_{VL}-HetFc |
| 55 | CL_#22206 | Briak_{VH}(F27V)-(G4S)3-Briak_{VL}-HetFc |
| 56 | CL_#22207 | Briak_{VH}(Y52AV)-(G4S)3-Briak_{VL}-HetFc |
| 57 | CL_#22208 | Briak_{VH}(R52E)-(G4S)3-Briak_{VL}-HetFc |
| 58 | CL_#22209 | Briak_{VH}(R52E_Y52AV)-(G4S)3-Briak_{VL}-HetFc |
| 59 | CL_#22211 | Briak_{VH}(H95D)-(G4S)3-Briak_{VL}-HetFc |
| 60 | CL_#22212 | Briak_{VH}(G96T)-(G4S)3-Briak_{VL}-HetFc |
| 61 | CL_#22214 | Briak_{VH}(H98A)-(G4S)3-Briak_{VL}-HetFc |
| 62 | CL_#22279 | HetFc-GSADGG-p40-(G4S)3-p35_{ΔR} |
| 63 | CL_#22289 | HetFc-GSADGG-p40_{Hep}-(G4S)3-p35_{ΔR} |
| 64 | CL_#22290 | HetFc-GSADGG-p40_{Hep}-(G4S)3-p35_{ΔR} |
| 65 | CL_#22291 | HetFc-GSADGG-p40_{Hep}-(G4S)3-p35_{ΔR} |
| 66 | CL_#22292 | HetFc-GSADGG-p40_{Hep}-(G4S)3-p35_{ΔR} |
| 67 | CL_#22293 | HetFc-GSADGG-p40_{Hep}-(G4S)3-p35_{ΔR} |
| 68 | CL_#22294 | HetFc-GSADGG-p40_{Hep}-(G4S)3-p35_{ΔR} |
| 69 | CL_#22295 | HetFc-GSADGG-p40_{Hep}-(G4S)3-p35_{ΔR} |
| 70 | CL_#22296 | HetFc-GSADGG-p40_{Hep}-(G4S)3-p35_{ΔR} |
| 71 | CL_#22672 | HetFc-(G4S)-MSGRSANA-(G4S)4-IL12Rβ2₂₄₋₃₂₁ |
| 72 | CL_#22735 | HetFc-(G4S)2-MSGRSANA-(G4S)2-Briak_{VH}-(G4S)- |
| | | MSGRSANA-(G4S)2-Briak_{VL} |
| 73 | CL_#23360 | HetFc-(G4S)2-MSGRSANA-(G4S)2-Briak_{VL}-(G2S-(G3S)4-G)- |
| | | Briak_{VH} |
| 74 | CL_#23361 | HetFc-(G4S)2-MSGRSANA-(G4S)2-Briak_{VL}(T100C)-(G2S- |
| | | (G3S)4-G)-Briak_{VH}(G44C) |
| 75 | CL_#23363 | HetFc-(G4S)2-MSGRSANA-(G4S)2-Briak_{VH}-(G2S-G3S- |
| | | MSGRSANA-(G3S)3-G)-Briak_{VL} |
| 76 | CL_#23364 | HetFc-(G4S)2-MSGRSANA-(G4S)2-Briak_{VH} (Y32A)-(G2S- |
| | | G3S-MSGRSANA-(G3S)3-G)-Briak_{VL} |
| 77 | CL_#23512 | HetFc-(G4S)2-(G3S)2-(G4S)2-Briak_{VH}-(G4S-(G3S)2-(G4S)2)- |
| | | Briak_{VL} |
| 78 | CL_#23513 | HetFc-G4S-(G3S)2-(G4S)4-IL12Rβ2₂₄₋₃₂₁ |
| 79 | CL_#23710 | HetFc-GSADGG-MSGRSANA-GSADGG-p40-(G4S)3-p35_{ΔR} |
| 80 | CL_#23711 | HetFc-GSADGG-MSGRSANA-GSADGG-p40_{Hep}-(G4S)3- |
| | | P35_{ΔR} |
| 81 | CL_#24228 | HetFc-GSADGG-p40-(G4S)3-p35_{ΔR}-G4S-MSGRSANA- |
| | | (G4S)4-h6F6_{VL}-GGS-(G3S)4-G-h6F6_{VH} |
| 82 | CL_#24229 | HetFc-(G4S)2-MSGRSANA-(G4S)2-Briak_{VH}-G4S-Mat- |
| | | (G4S)2-Briak_{VL}-(G4S)3-MSGRSANA-(G4S)3-h6F6_{VL}-GGS- |
| | | (G3S)4-G-h6F6_{VH} |
| 83 | CL_#24230 | HetFc-(G4S)2-MSGRSANA-(G4S)2-Briak_{VH}-G4S- |
| | | MSGRSANA-(G4S)2-Briak_{VL}-(G4S)3-MSGRSANA-(G4S)3- |
| | | h6F6_{VH}-GGS-(G3S)4-G-h6F6_{VH} |
| 84 | CL_#24231 | HetFc-GSADGG-p40-(G4S)3-p35_{ΔR}-G4S-MSGRSANA- |
| | | (G4S)5-IL12Rβ2₂₄₋₃₂₁ |
| 85 | CL_#24232 | HetFc-(G4S)3-MSGRSANA-(G4S)3-IL12Rβ1₂₄₋₂₄₀ |
| 86 | CL_#24233 | HetFc-G4S-MSGRSANA-(G4S)4-IL12Rβ2₂₄₋₁₂₄-(G4S)3- |
| | | MSGRSANA-(G4S)3-IL12Rβ1₂₄₋₂₄₀ |
| 87 | CL_#24235 | IL12Rβ2₂₄₋₁₂₄-(G4S)2-MSGRSANA-G4S-p35-(G4S)2-HetFc |
| 88 | CL_#24236 | IL12Rβ1₂₄₋₂₄₀-(G4S)4-MSGRSANA-HetFc |
| 89 | CL_#24246 | HetFc-G4S-MSGRSANA-(G4S)4-IL12Rβ2₂₄₋₁₂₄-(G4S)2- |
| | | MSGRSANA-G4S-p35 |
| 302 | CL_#23571 | HetFc-(G4S)2-MSGRSANA-(G4S)2-Briak_{VH}-(G4S)-(G3S)2- |
| | | (G4S)2-Briak_{VL} |
| 303 | CL_#24219 | HetFc-(G4S)2-MSGRSANA-G4S-Briak_{VH}-(G4S)- |
| | | MSGRSANA-(G4S)2-Briak_{VL} |
| 304 | CL_#24221 | HetFc-G4S-MSGRSANA-G4S-Briak_{VH}-(G4S)-MSGRSANA- |
| | | (G4S)2-Briak_{VL} |
| 305 | CL_#24222 | HetFc-GGS-MSGRSANA-GGS-Briak_{VH}-(G4S)-MSGRSANA- |
| | | (G4S)2-Briak_{VL} |
| 306 | CL_#24224 | HetFc-(G4S)2-MSGRSANA-(G4S)2-Briak_{VH}-GGS-(G3S)4-G- |
| | | Briak_{VL} |
| 307 | CL_#24308 | HetFc-G4S-MSGRSANA-(G4S)2-Briak_{VH}-(G4S)- |
| | | MSGRSANA-(G4S)2-Briak_{VL} |
| 308 | CL_#24831 | HetFc-GSADGG-p40(S175V_A179T_S183T_S294N)-(G4S)3- |
| | | P35β |
| 309 | CL_#24832 | HetFc-GSADGG-p40-(G4S)3-p35_{ΔR}(L68A) |
| 310 | CL_#24833 | HetFc-GSADGG-p40-(G4S)3-p35_{ΔR}(R181A) |
| 311 | CL_#24834 | HetFc-GSADGG-p40-(G4S)3-p35_{ΔR}(V185A) |
| 312 | CL_#24835 | HetFc-GSADGG-p40-(G4S)3-p35_{ΔR}(E38R_K128E_K168E) |
| 313 | CL_#24836 | HetFc-GSADGG-p40-(G4S)3-p35_{ΔR}(P41S_I171Q_I175S) |
| 314 | CL_#24837 | HetFc-GSADGG-p40-(G4S)3-p35_{ΔR}(F39S_Y40S_Y167S) |
| 315 | CL_#24838 | HetFc-GSADGG-p40-(G4S)3-p35_{ΔR}(T43A_E45R_I47S_D48R) |
| 316 | CL_#24839 | HetFc-GSADGG-p40-(G4S)3- |
| | | p35_{ΔR}(T43A_E45R_E46K_147S_D48R_E50K) |
| 317 | CL_#24840 | HetFc-GSADGG-p40(D41S_E45R_K58S_E59S_K195D)- |
| | | (G4S)3-p35_{ΔR} |
| 318 | CL_#24841 | HetFc-GSADGG-p40(K99S_E100S_R159S)-(G4S)3-p35_{ΔR} |
| 319 | CL_#24842 | HetFc-GSADGG-p40(E187S_T202S_S204E)-(G4S)3-p35_{ΔR} |
| 320 | CL_#26498 | HetFc-(G4S)2-MSGRSANA-(G4S)2-Briak_{VH}(F27V)-GGS- |
| | | (G3S)4-G-Briak_{VL} |
| 340 | CL_#26320 | HetFc-GSADGG-p40-(G4S)3-p35_{ΔR}-G4S-(G3S)2-(G4S)4- |
| | | h6F6_{VL}-GGS-(G3S)4-G-h6F6_{VH} |
| 341 | CL_#26503 | HetFc-(G4S)2-(G3S)2-(G4S)2-Briak_{VH}-GGS-(G3S)4-G-Briak_{VL} |

^{a}“HetFc” can indicate either chain A or B of a heterodimeric Fc, may or may not include a wild-type or modified IgG1 hinge, and may or may not include additional mutations in the CH2 and or CH3 domains;
^{b}“ΔR” in “p35_{ΔR}” indicates removal of p35 N-terminal Arg residue;
^{c}“Hep” in “p40_{Hep}” indicates a mutated heparin binding loop

TABLE 24
Sequences

| SEQ | Clone | | |
|---|---|---|---|
| ID | # | Descr. | Sequence (amino acid or nucleic acid) |
| 1 | | Signal Pep. | MRPTWAWWLFLVLLLALWAPARG |
| 2 | | PCS^{a} | TSGRSANP |
| 3 | | PCS | LSGRSDNH |
| 4 | | PCS | GSGRSAQV |
| 5 | | PCS | GSSRNADV |
| 6 | | PCS | GTARSDNV |
| 7 | | PCS | GGGRVNNV |
| 8 | | PCS | MSARILQV |
| 9 | | PCS | GKGRSANA |
| 10 | | PCS | MSGRSANA |
| 11 | | Bria^{b} VH | QVQLVESGGGVVQPGRSLRLSCAASGFTFSSYGMHWVRQAPGKGLEWVA |
| | | | FIRYDGSNKYYADSVKGRFTISRDNSKNTLYLQMNSLRAEDTAVYYCKTH |
| | | | GSHDNWGQGTMVTVSS |
| 12 | | Bria VL | QSVLTQPPSVSGAPGQRVTISCSGSRSNIGSNTVKWYQQLPGTAPKLLIYYN |
| | | | DQRPSGVPDRFSGSKSGTSASLAITGLQAEDEADYYCQSYDRYTHPALLFG |
| | | | TGTKVTVL |
| 13 | | Bria | SYGMH |
| | | VHCDR1 | |
| 14 | | Bria | FIRYDGSNKYYADSVKG |
| | | VHCDR2 | |
| 15 | | Bria | HGSHDN |
| | | VHCDR3 | |
| 16 | | Bria | SGSRSNIGSNTVK |
| | | VLCDR1 | |
| 17 | | Bria | YNDQRPS |
| | | VLCDR2 | |
| 18 | | Bria | QSYDRYTHPALL |
| | | VLCDR3 | |
| 19 | | IL12 Hep | QGKSKREKK |
| | | Binding | |
| | | Loop | |
| 20 | 12153 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPG |
| 21 | 12155 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPG |
| 22 | 17871 | Full AA | IWELKKDVYVVELDWYPDAPGEMVVLTCDTPEEDGITWTLDQSSEVLGSG |
| | | (heparin | KTLTIQVKEFGDAGQYTCHKGGEVLSHSLLLLHKKEDGIWSTDILKDQKEP |
| | | binding | KNKTFLRCEAKNYSGRFTCWWLTTISTDLTFSVKSSRGSSDPQGVTCGAAT |
| | | loop | LSAERVRGDNKEYEYSVECQEDSACPAAEESLPIEVMVDAVHKLKYENYT |
| | | underlined) | SSFFIRDIIKPDPPKNLQLKPLKNSRQVEVSWEYPDTWSTPHSYFSLTFCVQV |
| | | | QGKSKREKKDRVFTDKTSATVICRKNASISVRAQDRYYSSSWSEWASVPCS |
| 23 | 17872 | Full AA | RNLPVATPDPGMFPCLHHSQNLLRAVSNMLQKARQTLEFYPCTSEEIDHED |
| | | | ITKDKTSTVEACLPLELTKNESCLNSRETSFITNGSCLASRKTSFMMALCLSS |
| | | | IYEDLKMYQVEFKTMNAKLLMDPKRQIFLDQNMLAVIDELMQALNFNSET |
| | | | VPQKSSLEEPDFYKTKIKLCILLHAFRIRAVTIDRVMSYLNAS |
| 24 | 17875 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGRNLPVATPDPGMFPCLHHSQ |
| | | | NLLRAVSNMLQKARQTLEFYPCTSEEIDHEDITKDKTSTVEACLPLELTKNE |
| | | | SCLNSRETSFITNGSCLASRKTSFMMALCLSSIYEDLKMYQVEFKTMNAKL |
| | | | LMDPKRQIFLDQNMLAVIDELMQALNFNSETVPQKSSLEEPDFYKTKIKLCI |
| | | | LLHAFRIRAVTIDRVMSYLNAS |
| 25 | 17876 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSRNLPVATPDPGMFPCLHHSQNLLRAVSNMLQKARQTLEFYPCTSE |
| | | | EIDHEDITKDKTSTVEACLPLELTKNESCLNSRETSFITNGSCLASRKTSFMM |
| | | | ALCLSSIYEDLKMYQVEFKTMNAKLLMDPKRQIFLDQNMLAVIDELMQAL |
| | | | NFNSETVPQKSSLEEPDFYKTKIKLCILLHAFRIRAVTIDRVMSYLNAS |
| 26 | 17877 | Full AA | RNLPVATPDPGMFPCLHHSQNLLRAVSNMLQKARQTLEFYPCTSEEIDHED |
| | | | ITKDKTSTVEACLPLELTKNESCLNSRETSFITNGSCLASRKTSFMMALCLSS |
| | | | IYEDLKMYQVEFKTMNAKLLMDPKRQIFLDQNMLAVIDELMQALNFNSET |
| | | | VPQKSSLEEPDFYKTKIKLCILLHAFRIRAVTIDRVMSYLNASGGGGSGGGG |
| | | | SEPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPG |
| 27 | 17879 | Full AA | IWELKKDVYVVELDWYPDAPGEMVVLTCDTPEEDGITWTLDQSSEVLGSG |
| | | | KTLTIQVKEFGDAGQYTCHKGGEVLSHSLLLLHKKEDGIWSTDILKDQKEP |
| | | | KNKTFLRCEAKNYSGRFTCWWLTTISTDLTFSVKSSRGSSDPQGVTCGAAT |
| | | | LSAERVRGDNKEYEYSVECQEDSACPAAEESLPIEVMVDAVHKLKYENYT |
| | | | SSFFIRDIIKPDPPKNLQLKPLKNSRQVEVSWEYPDTWSTPHSYFSLTFCVQV |
| | | | QGKSKREKKDRVFTDKTSATVICRKNASISVRAQDRYYSSSWSEWASVPCS |
| | | | GGGGSGGGGSEPKSSDKTHTCPPCPAPEAAGGPSVFLFPPKPKDTLMISRTP |
| | | | EVTCVVVSVSHEDPEVKFNWYVDGVEVHNAKTKPREEQYNSTYRVVSVL |
| | | | TVLHQDWLNGKEYKCKVSNKALPAPIEKTISKAKGQPREPQVYVYPPSRD |
| | | | ELTKNQVSLTCLVKGFYPSDIAVEWESNGQPENNYKTTPPVLDSDGSFALV |
| | | | SKLTVDKSRWQQGNVFSCSVMHEALHNHYTQKSLSLSPG |
| 28 | 17880 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSIWELKKDVY |
| | | | VVELDWYPDAPGEMVVLTCDTPEEDGITWTLDQSSEVLGSGKTLTIQVKEF |
| | | | GDAGQYTCHKGGEVLSHSLLLLHKKEDGIWSTDILKDQKEPKNKTFLRCE |
| | | | AKNYSGRFTCWWLTTISTDLTFSVKSSRGSSDPQGVTCGAATLSAERVRGD |
| | | | NKEYEYSVECQEDSACPAAEESLPIEVMVDAVHKLKYENYTSSFFIRDIIKP |
| | | | DPPKNLQLKPLKNSRQVEVSWEYPDTWSTPHSYFSLTFCVQVQGKSKREK |
| | | | KDRVFTDKTSATVICRKNASISVRAQDRYYSSSWSEWASVPCS |
| 29 | 17881 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGRNLPVATPDPGMFPCLHHSQ |
| | | | NLLRAVSNMLQKARQTLEFYPCTSEEIDHEDITKDKTSTVEACLPLELTKNE |
| | | | SCLNSRETSFITNGSCLASRKTSFMMALCLSSIYEDLKMYQVEFKTMNAKL |
| | | | LMDPKRQIFLDQNMLAVIDELMQALNFNSETVPQKSSLEEPDFYKTKIKLCI |
| | | | LLHAFRIRAVTIDRVMSYLNAS |
| 30 | 17906 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGRAVPGGSSPAWTQCQQLSQ |
| | | | KLCTLAWSAHPLVGHMDLREEGDEETTNDVPHIQCGDGCDPQGLRDNSQF |
| | | | CLQRIHQGLIFYEKLLGSDIFTGEPSLLPDSPVGQLHASLLGLSQLLQPEGHH |
| | | | WETQQIPSLSPSQPWQRLLLRFKILRSLQAFVAVAARVFAHGAATLSP |
| 31 | 17907 | Full AA | RAVPGGSSPAWTQCQQLSQKLCTLAWSAHPLVGHMDLREEGDEETTNDV |
| | | | PHIQCGDGCDPQGLRDNSQFCLQRIHQGLIFYEKLLGSDIFTGEPSLLPDSPV |
| | | | GQLHASLLGLSQLLQPEGHHWETQQIPSLSPSQPWQRLLLRFKILRSLQAFV |
| | | | AVAARVFAHGAATLSPGGGGSGGGGSEPKSSDKTHTCPPCPAPEAAGGPS |
| | | | VFLFPPKPKDTLMISRTPEVTCVVVSVSHEDPEVKFNWYVDGVEVHNAKT |
| | | | KPREEQYNSTYRVVSVLTVLHQDWLNGKEYKCKVSNKALPAPIEKTISKA |
| | | | KGQPREPQVYVYPPSRDELTKNQVSLTCLVKGFYPSDIAVEWESNGQPENN |
| | | | YKTTPPVLDSDGSFALVSKLTVDKSRWQQGNVFSCSVMHEALHNHYTQKS |
| | | | LSLSPG |
| 32 | 17908 | Full AA | RAVPGGSSPAWTQCQQLSQKLCTLAWSAHPLVGHMDLREEGDEETTNDV |
| | | | PHIQCGDGCDPQGLRDNSQFCLQRIHQGLIFYEKLLGSDIFTGEPSLLPDSPV |
| | | | GQLHASLLGLSQLLQPEGHHWETQQIPSLSPSQPWQRLLLRFKILRSLQAFV |
| | | | AVAARVFAHGAATLSP |
| 33 | 17942 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGGGGSGGGGSIWELKKDVY |
| | | | VVELDWYPDAPGEMVVLTCDTPEEDGITWTLDQSSEVLGSGKTLTIQVKEF |
| | | | GDAGQYTCHKGGEVLSHSLLLLHKKEDGIWSTDILKDQKEPKNKTFLRCE |
| | | | AKNYSGRFTCWWLTTISTDLTFSVKSSRGSSDPQGVTCGAATLSAERVRGD |
| | | | NKEYEYSVECQEDSACPAAEESLPIEVMVDAVHKLKYENYTSSFFIRDIIKP |
| | | | DPPKNLQLKPLKNSRQVEVSWEYPDTWSTPHSYFSLTFCVQVQGKSKREK |
| | | | KDRVFTDKTSATVICRKNASISVRAQDRYYSSSWSEWASVPCS |
| 34 | 17945 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSGGGGSRAVPGGSSPAWTQCQQLSQKLCTLAWSAHPLVGHMDLRE |
| | | | EGDEETTNDVPHIQCGDGCDPQGLRDNSQFCLQRIHQGLIFYEKLLGSDIFT |
| | | | GEPSLLPDSPVGQLHASLLGLSQLLQPEGHHWETQQIPSLSPSQPWQRLLLR |
| | | | FKILRSLQAFVAVAARVFAHGAATLSP |
| 35 | 18939 | Full AA | QVQLVESGGGVVQPGRSLRLSCAASGFTFSSYGMHWVRQAPGKGLEWVA |
| | | | FIRYDGSNKYYADSVKGRFTISRDNSKNTLYLQMNSLRAEDTAVYYCKTH |
| | | | GSHDNWGQGTMVTVSSASTKGPSVFPLAPSSKSTSGGTAALGCLVKDYFP |
| | | | EPVTVSWNSGALTSGVHTFPAVLQSSGLYSLSSVVTVPSSSLGTQTYICNVN |
| | | | HKPSNTKVDKRVEPKSCDKTHTCPPCPAPEAAGGPSVFLFPPKPKDTLMISR |
| | | | TPEVTCVVVSVSHEDPEVKFNWYVDGVEVHNAKTKPREEQYNSTYRVVS |
| | | | VLTVLHQDWLNGKEYKCKVSNKALPAPIEKTISKAKGQPREPQVYVLPPSR |
| | | | DELTKNQVSLLCLVKGFYPSDIAVEWESNGQPENNYLTWPPVLDSDGSFFL |
| | | | YSKLTVDKSRWQQGNVFSCSVMHEALHNHYTQKSLSLSPG |
| 36 | 18940 | Full AA | QSVLTQPPSVSGAPGQRVTISCSGSRSNIGSNTVKWYQQLPGTAPKLLIYYN |
| | | | DQRPSGVPDRFSGSKSGTSASLAITGLQAEDEADYYCQSYDRYTHPALLFG |
| | | | TGTKVTVLGQPKAAPSVTLFPPSSEELQANKATLVCLISDFYPGAVTVAWK |
| | | | ADSSPVKAGVETTTPSKQSNNKYAASSYLSLTPEQWKSHRSYSCQVTHEGS |
| | | | TVEKTVAPAECS |
| 37 | 18942 | Full AA | QVQLVESGGGVVQPGRSLRLSCAASGFTFSSYGMHWVRQAPGKGLEWVA |
| | | | FIRYDGSNKYYADSVKGRFTISRDNSKNTLYLQMNSLRAEDTAVYYCKTH |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLAAEPKSSDKT |
| | | | HTCPPCPAPEAAGGPSVFLFPPKPKDTLMISRTPEVTCVVVSVSHEDPEVKF |
| | | | NWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPS |
| | | | DIAVEWESNGQPENNYLTWPPVLDSDGSFFLYSKLTVDKSRWQQGNVFSC |
| | | | SVMHEALHNHYTQKSLSLSPG |
| 38 | 18943 | Full AA | QSVLTQPPSVSGAPGQRVTISCSGSRSNIGSNTVKWYQQLPGTAPKLLIYYN |
| | | | DQRPSGVPDRFSGSKSGTSASLAITGLQAEDEADYYCQSYDRYTHPALLFG |
| | | | TGTKVTVLGGGGSGGGGSGGGGSQVQLVESGGGVVQPGRSLRLSCAASGF |
| | | | TFSSYGMHWVRQAPGKGLEWVAFIRYDGSNKYYADSVKGRFTISRDNSK |
| | | | NTLYLQMNSLRAEDTAVYYCKTHGSHDNWGQGTMVTVSSAAEPKSSDKT |
| | | | HTCPPCPAPEAAGGPSVFLFPPKPKDTLMISRTPEVTCVVVSVSHEDPEVKF |
| | | | NWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPS |
| | | | DIAVEWESNGQPENNYLTWPPVLDSDGSFFLYSKLTVDKSRWQQGNVFSC |
| | | | SVMHEALHNHYTQKSLSLSPG |
| 39 | 18953 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSLSGRSDNHGGGGSG |
| | | | GGGSGGGGSGGGGSKIDACKRGDVTVKPSHVILLGSTVNITCSLKPRQGCF |
| | | | HYSRRNKLILYKFDRRINFHHGHSLNSQVTGLPLGTTLFVCKLACINSDEIQI |
| | | | CGAEIFVGVAPEQPQNLSCIQKGEQGTVACTWERGRDTHLYTEYTLQLSGP |
| | | | KNLTWQKQCKDIYCDYLDFGINLTPESPESNFTAKVTAVNSLGSSSSLPSTF |
| | | | TFLDIVRPLPPWDIRIKFQKASVSRCTLYWRDEGLVLLNRLRYRPSNSRLW |
| | | | NMVNVTKAKGRHDLLDLKPFTEYEFQISSKLHLYKGSWSDWSESLRAQTP |
| | | | EEEP |
| 40 | 18954 | Full AA | KIDACKRGDVTVKPSHVILLGSTVNITCSLKPRQGCFHYSRRNKLILYKFDR |
| | | | RINFHHGHSLNSQVTGLPLGTTLFVCKLACINSDEIQICGAEIFVGVAPEQPQ |
| | | | NLSCIQKGEQGTVACTWERGRDTHLYTEYTLQLSGPKNLTWQKQCKDIYC |
| | | | DYLDFGINLTPESPESNFTAKVTAVNSLGSSSSLPSTFTFLDIVRPLPPWDIRI |
| | | | KFQKASVSRCTLYWRDEGLVLLNRLRYRPSNSRLWNMVNVTKAKGRHDL |
| | | | LDLKPFTEYEFQISSKLHLYKGSWSDWSESLRAQTPEEEPGGGGSGGGGSL |
| | | | SGRSDNHGGGGSEPKSSDKTHTCPPCPAPEAAGGPSVFLFPPKPKDTLMISR |
| | | | TPEVTCVVVSVSHEDPEVKFNWYVDGVEVHNAKTKPREEQYNSTYRVVS |
| | | | VLTVLHQDWLNGKEYKCKVSNKALPAPIEKTISKAKGQPREPQVYVLPPSR |
| | | | DELTKNQVSLLCLVKGFYPSDIAVEWESNGQPENNYLTWPPVLDSDGSFFL |
| | | | YSKLTVDKSRWQQGNVFSCSVMHEALHNHYTQKSLSLSPG |
| 41 | 18956 | Full AA | KIDACKRGDVTVKPSHVILLGSTVNITCSLKPRQGCFHYSRRNKLILYKFDR |
| | | | RINFHHGHSLNSQVTGLPLGTTLFVCKLACINSDEIQICGAEIFVGVAPGGG |
| | | | GSGGGGSLSGRSDNHGGGGSRNLPVATPDPGMFPCLHHSQNLLRAVSNML |
| | | | QKARQTLEFYPCTSEEIDHEDITKDKTSTVEACLPLELTKNESCLNSRETSFI |
| | | | TNGSCLASRKTSFMMALCLSSIYEDLKMYQVEFKTMNAKLLMDPKRQIFL |
| | | | DQNMLAVIDELMQALNFNSETVPQKSSLEEPDFYKTKIKLCILLHAFRIRAV |
| | | | TIDRVMSYLNASGGGGSGGGGSEPKSSDKTHTCPPCPAPEAAGGPSVFLFPP |
| | | | KPKDTLMISRTPEVTCVVVSVSHEDPEVKFNWYVDGVEVHNAKTKPREEQ |
| | | | YNSTYRVVSVLTVLHQDWLNGKEYKCKVSNKALPAPIEKTISKAKGQPRE |
| | | | PQVYVYPPSRDELTKNQVSLTCLVKGFYPSDIAVEWESNGQPENNYKTTPP |
| | | | VLDSDGSFALVSKLTVDKSRWQQGNVFSCSVMHEALHNHYTQKSLSLSPG |
| 42 | 18957 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSRNLPVATPDPGMFPCLHHSQNLLRAVSNMLQKARQTLEFYPCTSE |
| | | | EIDHEDITKDKTSTVEACLPLELTKNESCLNSRETSFITNGSCLASRKTSFMM |
| | | | ALCLSSIYEDLKMYQVEFKTMNAKLLMDPKRQIFLDQNMLAVIDELMQAL |
| | | | NFNSETVPQKSSLEEPDFYKTKIKLCILLHAFRIRAVTIDRVMSYLNASGGG |
| | | | GSLSGRSDNHGGGGSGGGGSKIDACKRGDVTVKPSHVILLGSTVNITCSLK |
| | | | PRQGCFHYSRRNKLILYKFDRRINFHHGHSLNSQVTGLPLGTTLFVCKLACI |
| | | | NSDEIQICGAEIFVGVAP |
| 43 | 21415 | Full AA | IWELKKDVYVVELDWYPDAPGEMVVLTCDTPEEDGITWTLDQSSEVLGSG |
| | | | KTLTIQVKEFGDAGQYTCHKGGEVLSHSLLLLHKKEDGIWSTDILKDQKEP |
| | | | KNKTFLRCEAKNYSGRFTCWWLTTISTDLTFSVKSSRGSSDPQGVTCGAAT |
| | | | LSAERVRGDNKEYEYSVECQEDSACPAAEESLPIEVMVDAVHKLKYENYT |
| | | | SSFFIRDIIKPDPPKNLQLKPLKNSRQVEVSWEYPDTWSTPHSYFSLTFCVQV |
| | | | QGKSKREKKDRVFTDKTSATVICRKNASISVRAQDRYYSSSWSEWASVPCS |
| | | | GGGGSGGGGSMSGRSANAGGGGSGGGGSGGGGSQSVLTQPPSVSGAPGQ |
| | | | RVTISCSGSRSNIGSNTVKWYQQLPGTAPKLLIYYNDQRPSGVPDRFSGSKS |
| | | | GTSASLAITGLQAEDEADYYCQSYDRYTHPALLFGTGTKVTVLGGGGSGG |
| | | | GGSGGGGSQVQLVESGGGVVQPGRSLRLSCAASGFTFSSYGMHWVRQAP |
| | | | GKGLEWVAFIRYDGSNKYYADSVKGRFTISRDNSKNTLYLQMNSLRAEDT |
| | | | AVYYCKTHGSHDNWGQGTMVTVSS |
| 44 | 21416 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGRNLPVATPDPGMFPCLHHSQ |
| | | | NLLRAVSNMLQKARQTLEFYPCTSEEIDHEDITKDKTSTVEACLPLELTKNE |
| | | | SCLNSRETSFITNGSCLASRKTSFMMALCLSSIYEDLKMYQVEFKTMNAKL |
| | | | LMDPKRQIFLDQNMLAVIDELMQALNFNSETVPQKSSLEEPDFYKTKIKLCI |
| | | | LLHAFRIRAVTIDRVMSYLNASGGGGSGGGGSMSGRSANAGGGGSGGGGS |
| | | | GGGGSQSVLTQPPSVSGAPGQRVTISCSGSRSNIGSNTVKWYQQLPGTAPK |
| | | | LLIYYNDQRPSGVPDRFSGSKSGTSASLAITGLQAEDEADYYCQSYDRYTH |
| | | | PALLFGTGTKVTVLGGGGSGGGGSGGGGSQVQLVESGGGVVQPGRSLRLS |
| | | | CAASGFTFSSYGMHWVRQAPGKGLEWVAFIRYDGSNKYYADSVKGRFTIS |
| | | | RDNSKNTLYLQMNSLRAEDTAVYYCKTHGSHDNWGQGTMVTVSS |
| 45 | 21417 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSGGGGSQSVLTQPPSVSGAPGQRVTISCSGSRSNIGSNTVKWYQQLPG |
| | | | TAPKLLIYYNDQRPSGVPDRFSGSKSGTSASLAITGLQAEDEADYYCQSYD |
| | | | RYTHPALLFGTGTKVTVLGGGGSGGGGSGGGGSQVQLVESGGGVVQPGR |
| | | | SLRLSCAASGFTFSSYGMHWVRQAPGKGLEWVAFIRYDGSNKYYADSVK |
| | | | GRFTISRDNSKNTLYLQMNSLRAEDTAVYYCKTHGSHDNWGQGTMVTVS |
| | | | S |
| 46 | 21418 | Full AA | QVQLVESGGGVVQPGRSLRLSCAASGFTFSSYGMHWVRQAPGKGLEWVA |
| | | | FIRYDGSNKYYADSVKGRFTISRDNSKNTLYLQMNSLRAEDTAVYYCKTH |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLGGGGSGGGG |
| | | | SGGGGSMSGRSANAGGGGSGGGGSIWELKKDVYVVELDWYPDAPGEMV |
| | | | VLTCDTPEEDGITWTLDQSSEVLGSGKTLTIQVKEFGDAGQYTCHKGGEVL |
| | | | SHSLLLLHKKEDGIWSTDILKDQKEPKNKTFLRCEAKNYSGRFTCWWLTTI |
| | | | STDLTFSVKSSRGSSDPQGVTCGAATLSAERVRGDNKEYEYSVECQEDSAC |
| | | | PAAEESLPIEVMVDAVHKLKYENYTSSFFIRDIIKPDPPKNLQLKPLKNSRQ |
| | | | VEVSWEYPDTWSTPHSYFSLTFCVQVQGKSKREKKDRVFTDKTSATVICR |
| | | | KNASISVRAQDRYYSSSWSEWASVPCSGGGGSGGGGSEPKSSDKTHTCPPC |
| | | | PAPEAAGGPSVFLFPPKPKDTLMISRTPEVTCVVVSVSHEDPEVKFNWYVD |
| | | | GVEVHNAKTKPREEQYNSTYRVVSVLTVLHQDWLNGKEYKCKVSNKALP |
| | | | APIEKTISKAKGQPREPQVYVYPPSRDELTKNQVSLTCLVKGFYPSDIAVEW |
| | | | ESNGQPENNYKTTPPVLDSDGSFALVSKLTVDKSRWQQGNVFSCSVMHEA |
| | | | LHNHYTQKSLSLSPG |
| 47 | 21419 | Full AA | RNLPVATPDPGMFPCLHHSQNLLRAVSNMLQKARQTLEFYPCTSEEIDHED |
| | | | ITKDKTSTVEACLPLELTKNESCLNSRETSFITNGSCLASRKTSFMMALCLSS |
| | | | IYEDLKMYQVEFKTMNAKLLMDPKRQIFLDQNMLAVIDELMQALNFNSET |
| | | | VPQKSSLEEPDFYKTKIKLCILLHAFRIRAVTIDRVMSYLNASGGGGSGGGG |
| | | | SMSGRSANAGGGGSGGGGSGGGGSQSVLTQPPSVSGAPGQRVTISCSGSRS |
| | | | NIGSNTVKWYQQLPGTAPKLLIYYNDQRPSGVPDRFSGSKSGTSASLAITGL |
| | | | QAEDEADYYCQSYDRYTHPALLFGTGTKVTVLGGGGSGGGGSGGGGSQV |
| | | | QLVESGGGVVQPGRSLRLSCAASGFTFSSYGMHWVRQAPGKGLEWVAFIR |
| | | | YDGSNKYYADSVKGRFTISRDNSKNTLYLQMNSLRAEDTAVYYCKTHGS |
| | | | HDNWGQGTMVTVSS |
| 48 | 21421 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGGGGSGGGGSIWELKKDVY |
| | | | VVELDWYPDAPGEMVVLTCDTPEEDGITWTLDQSSEVLGSGKTLTIQVKEF |
| | | | GDAGQYTCHKGGEVLSHSLLLLHKKEDGIWSTDILKDQKEPKNKTFLRCE |
| | | | AKNYSGRFTCWWLTTISTDLTFSVKSSRGSSDPQGVTCGAATLSAERVRGD |
| | | | NKEYEYSVECQEDSACPAAEESLPIEVMVDAVHKLKYENYTSSFFIRDIIKP |
| | | | DPPKNLQLKPLKNSRQVEVSWEYPDTWSTPHSYFSLTFCVQVQGKSKREK |
| | | | KDRVFTDKTSATVICRKNASISVRAQDRYYSSSWSEWASVPCSGGGGSGG |
| | | | GGSMSGRSANAGGGGSGGGGSGGGGSQSVLTQPPSVSGAPGQRVTISCSG |
| | | | SRSNIGSNTVKWYQQLPGTAPKLLIYYNDQRPSGVPDRFSGSKSGTSASLAI |
| | | | TGLQAEDEADYYCQSYDRYTHPALLFGTGTKVTVLGGGGSGGGGSGGGG |
| | | | SQVQLVESGGGVVQPGRSLRLSCAASGFTFSSYGMHWVRQAPGKGLEWV |
| | | | AFIRYDGSNKYYADSVKGRFTISRDNSKNTLYLQMNSLRAEDTAVYYCKT |
| | | | HGSHDNWGQGTMVTVSS |
| 49 | 21423 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSRNLPVATPDPGMFPCLHHSQNLLRAVSNMLQKARQTLEFYPCTSE |
| | | | EIDHEDITKDKTSTVEACLPLELTKNESCLNSRETSFITNGSCLASRKTSFMM |
| | | | ALCLSSIYEDLKMYQVEFKTMNAKLLMDPKRQIFLDQNMLAVIDELMQAL |
| | | | NFNSETVPQKSSLEEPDFYKTKIKLCILLHAFRIRAVTIDRVMSYLNASGGG |
| | | | GSGGGGSGGGGSMSGRSANAGGGGSGGGGSGGGGSGGGGSQSVLTQPPS |
| | | | VSGAPGQRVTISCSGSRSNIGSNTVKWYQQLPGTAPKLLIYYNDQRPSGVP |
| | | | DRFSGSKSGTSASLAITGLQAEDEADYYCQSYDRYTHPALLFGTGTKVTVL |
| | | | GGGGSGGGGSGGGGSQVQLVESGGGVVQPGRSLRLSCAASGFTFSSYGMH |
| | | | WVRQAPGKGLEWVAFIRYDGSNKYYADSVKGRFTISRDNSKNTLYLQMN |
| | | | SLRAEDTAVYYCKTHGSHDNWGQGTMVTVSS |
| 50 | 21446 | Full AA | QVQLVESGGGVVQPGRSLRLSCAASGFTFSSYGMHWVRQAPGKGLEWVA |
| | | | FIRYDGSNKYYADSVKGRFTISRDNSKNTLYLQMNSLRAEDTAVYYCKTH |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLGGGGSGGGG |
| | | | SMSGRSANAGGGGSGGGGSIWELKKDVYVVELDWYPDAPGEMVVLTCD |
| | | | TPEEDGITWTLDQSSEVLGSGKTLTIQVKEFGDAGQYTCHKGGEVLSHSLL |
| | | | LLHKKEDGIWSTDILKDQKEPKNKTFLRCEAKNYSGRFTCWWLTTISTDLT |
| | | | FSVKSSRGSSDPQGVTCGAATLSAERVRGDNKEYEYSVECQEDSACPAAEE |
| | | | SLPIEVMVDAVHKLKYENYTSSFFIRDIIKPDPPKNLQLKPLKNSRQVEVSW |
| | | | EYPDTWSTPHSYFSLTFCVQVQGKSKREKKDRVFTDKTSATVICRKNASIS |
| | | | VRAQDRYYSSSWSEWASVPCS |
| 51 | 21447 | Full AA | QVQLVESGGGVVQPGRSLRLSCAASGFTFSSYGMHWVRQAPGKGLEWVA |
| | | | FIRYDGSNKYYADSVKGRFTISRDNSKNTLYLQMNSLRAEDTAVYYCKTH |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLGGGGSGGGG |
| | | | SGGGGSGGGGSMSGRSANAGGGGSGGGGSGGGGSRNLPVATPDPGMFPC |
| | | | LHHSQNLLRAVSNMLQKARQTLEFYPCTSEEIDHEDITKDKTSTVEACLPLE |
| | | | LTKNESCLNSRETSFITNGSCLASRKTSFMMALCLSSIYEDLKMYQVEFKT |
| | | | MNAKLLMDPKRQIFLDQNMLAVIDELMQALNFNSETVPQKSSLEEPDFYK |
| | | | TKIKLCILLHAFRIRAVTIDRVMSYLNAS |
| 52 | 21451 | Full AA | QVQLVESGGGVVQPGRSLRLSCAASGFTFSSYGMHWVRQAPGKGLEWVA |
| | | | FIRYDGSNKYYADSVKGRFTISRDNSKNTLYLQMNSLRAEDTAVYYCKTH |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLGGGGSGGGG |
| | | | SGGGGSGGGGSMSGRSANAGGGGSGGGGSGGGGSEPKSSDKTHTCPPCPA |
| | | | PEAAGGPSVFLFPPKPKDTLMISRTPEVTCVVVSVSHEDPEVKFNWYVDGV |
| | | | EVHNAKTKPREEQYNSTYRVVSVLTVLHQDWLNGKEYKCKVSNKALPAPI |
| | | | EKTISKAKGQPREPQVYVLPPSRDELTKNQVSLLCLVKGFYPSDIAVEWES |
| | | | NGQPENNYLTWPPVLDSDGSFFLYSKLTVDKSRWQQGNVFSCSVMHEAL |
| | | | HNHYTQKSLSLSPG |
| 53 | 21452 | Full AA | QVQLVESGGGVVQPGRSLRLSCAASGFTFSSYGMHWVRQAPGKGLEWVA |
| | | | FIRYDGSNKYYADSVKGRFTISRDNSKNTLYLQMNSLRAEDTAVYYCKTH |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLGGGGSGGGG |
| | | | SGGGGSMSGRSANAGGGGSGGGGSGGGGSGGGGSRNLPVATPDPGMFPC |
| | | | LHHSQNLLRAVSNMLQKARQTLEFYPCTSEEIDHEDITKDKTSTVEACLPLE |
| | | | LTKNESCLNSRETSFITNGSCLASRKTSFMMALCLSSIYEDLKMYQVEFKT |
| | | | MNAKLLMDPKRQIFLDQNMLAVIDELMQALNFNSETVPQKSSLEEPDFYK |
| | | | TKIKLCILLHAFRIRAVTIDRVMSYLNASGGGGSGGGGSEPKSSDKTHTCPP |
| | | | CPAPEAAGGPSVFLFPPKPKDTLMISRTPEVTCVVVSVSHEDPEVKFNWYV |
| | | | DGVEVHNAKTKPREEQYNSTYRVVSVLTVLHQDWLNGKEYKCKVSNKAL |
| | | | PAPIEKTISKAKGQPREPQVYVYPPSRDELTKNQVSLTCLVKGFYPSDIAVE |
| | | | WESNGQPENNYKTTPPVLDSDGSFALVSKLTVDKSRWQQGNVFSCSVMH |
| | | | EALHNHYTQKSLSLSPG |
| 54 | 22203 | Full AA | QVQLVESGGGVVQPGRSLRLSCAASGFTFSSAGMHWVRQAPGKGLEWVA |
| | | | FIRYDGSNKYYADSVKGRFTISRDNSKNTLYLQMNSLRAEDTAVYYCKTH |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLAAEPKSSDKT |
| | | | HTCPPCPAPEAAGGPSVFLFPPKPKDTLMISRTPEVTCVVVSVSHEDPEVKF |
| | | | NWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPS |
| | | | DIAVEWESNGQPENNYLTWPPVLDSDGSFFLYSKLTVDKSRWQQGNVFSC |
| | | | SVMHEALHNHYTQKSLSLSPG |
| 55 | 22206 | Full AA | QVQLVESGGGVVQPGRSLRLSCAASGVTFSSYGMHWVRQAPGKGLEWVA |
| | | | FIRYDGSNKYYADSVKGRFTISRDNSKNTLYLQMNSLRAEDTAVYYCKTH |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLAAEPKSSDKT |
| | | | HTCPPCPAPEAAGGPSVFLFPPKPKDTLMISRTPEVTCVVVSVSHEDPEVKF |
| | | | NWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPS |
| | | | DIAVEWESNGQPENNYLTWPPVLDSDGSFFLYSKLTVDKSRWQQGNVFSC |
| | | | SVMHEALHNHYTQKSLSLSPG |
| 56 | 22207 | Full AA | QVQLVESGGGVVQPGRSLRLSCAASGFTFSSYGMHWVRQAPGKGLEWVA |
| | | | FIRVDGSNKYYADSVKGRFTISRDNSKNTLYLQMNSLRAEDTAVYYCKTH |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLAAEPKSSDKT |
| | | | HTCPPCPAPEAAGGPSVFLFPPKPKDTLMISRTPEVTCVVVSVSHEDPEVKF |
| | | | NWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPS |
| | | | DIAVEWESNGQPENNYLTWPPVLDSDGSFFLYSKLTVDKSRWQQGNVFSC |
| | | | SVMHEALHNHYTQKSLSLSPG |
| 57 | 22208 | Full AA | QVQLVESGGGVVQPGRSLRLSCAASGFTFSSYGMHWVRQAPGKGLEWVA |
| | | | FIEYDGSNKYYADSVKGRFTISRDNSKNTLYLQMNSLRAEDTAVYYCKTH |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLAAEPKSSDKT |
| | | | HTCPPCPAPEAAGGPSVFLFPPKPKDTLMISRTPEVTCVVVSVSHEDPEVKF |
| | | | NWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPS |
| | | | DIAVEWESNGQPENNYLTWPPVLDSDGSFFLYSKLTVDKSRWQQGNVFSC |
| | | | SVMHEALHNHYTQKSLSLSPG |
| 58 | 22209 | Full AA | QVQLVESGGGVVQPGRSLRLSCAASGFTFSSYGMHWVRQAPGKGLEWVA |
| | | | FIEVDGSNKYYADSVKGRFTISRDNSKNTLYLQMNSLRAEDTAVYYCKTH |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLAAEPKSSDKT |
| | | | HTCPPCPAPEAAGGPSVFLFPPKPKDTLMISRTPEVTCVVVSVSHEDPEVKF |
| | | | NWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPS |
| | | | DIAVEWESNGQPENNYLTWPPVLDSDGSFFLYSKLTVDKSRWQQGNVFSC |
| | | | SVMHEALHNHYTQKSLSLSPG |
| 59 | 22211 | Full AA | QVQLVESGGGVVQPGRSLRLSCAASGFTFSSYGMHWVRQAPGKGLEWVA |
| | | | FIRYDGSNKYYADSVKGRFTISRDNSKNTLYLQMNSLRAEDTAVYYCKTD |
| | | | GSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLAAEPKSSDKT |
| | | | HTCPPCPAPEAAGGPSVFLFPPKPKDTLMISRTPEVTCVVVSVSHEDPEVKF |
| | | | NWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPS |
| | | | DIAVEWESNGQPENNYLTWPPVLDSDGSFFLYSKLTVDKSRWQQGNVFSC |
| | | | SVMHEALHNHYTQKSLSLSPG |
| 60 | 22212 | Full AA | QVQLVESGGGVVQPGRSLRLSCAASGFTFSSYGMHWVRQAPGKGLEWVA |
| | | | FIRYDGSNKYYADSVKGRFTISRDNSKNTLYLQMNSLRAEDTAVYYCKTH |
| | | | TSHDNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLAAEPKSSDKT |
| | | | HTCPPCPAPEAAGGPSVFLFPPKPKDTLMISRTPEVTCVVVSVSHEDPEVKF |
| | | | NWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPS |
| | | | DIAVEWESNGQPENNYLTWPPVLDSDGSFFLYSKLTVDKSRWQQGNVFSC |
| | | | SVMHEALHNHYTQKSLSLSPG |
| 61 | 22214 | Full AA | QVQLVESGGGVVQPGRSLRLSCAASGFTFSSYGMHWVRQAPGKGLEWVA |
| | | | FIRYDGSNKYYADSVKGRFTISRDNSKNTLYLQMNSLRAEDTAVYYCKTH |
| | | | GSADNWGQGTMVTVSSGGGGSGGGGSGGGGSQSVLTQPPSVSGAPGQRV |
| | | | TISCSGSRSNIGSNTVKWYQQLPGTAPKLLIYYNDQRPSGVPDRFSGSKSGT |
| | | | SASLAITGLQAEDEADYYCQSYDRYTHPALLFGTGTKVTVLAAEPKSSDKT |
| | | | HTCPPCPAPEAAGGPSVFLFPPKPKDTLMISRTPEVTCVVVSVSHEDPEVKF |
| | | | NWYVDGVEVHNAKTKPREEQYNSTYRVVSVLTVLHQDWLNGKEYKCKV |
| | | | SNKALPAPIEKTISKAKGQPREPQVYVLPPSRDELTKNQVSLLCLVKGFYPS |
| | | | DIAVEWESNGQPENNYLTWPPVLDSDGSFFLYSKLTVDKSRWQQGNVFSC |
| | | | SVMHEALHNHYTQKSLSLSPG |
| 62 | 22279 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYPCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFYKTKIKLCILLHAFRIRAVTIDRVMSYLNAS |
| 63 | 22289 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVRDDSEDRVFTD |
| | | | KTSATVICRKNASISVRAQDRYYSSSWSEWASVPCSGGGGSGGGGSGGGG |
| | | | SNLPVATPDPGMFPCLHHSQNLLRAVSNMLQKARQTLEFYPCTSEEIDHED |
| | | | ITKDKTSTVEACLPLELTKNESCLNSRETSFITNGSCLASRKTSFMMALCLSS |
| | | | IYEDLKMYQVEFKTMNAKLLMDPKRQIFLDQNMLAVIDELMQALNFNSET |
| | | | VPQKSSLEEPDFYKTKIKLCILLHAFRIRAVTIDRVMSYLNAS |
| 64 | 22290 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGSQEKKDRVF |
| | | | TDKTSATVICRKNASISVRAQDRYYSSSWSEWASVPCSGGGGSGGGGSGG |
| | | | GGSNLPVATPDPGMFPCLHHSQNLLRAVSNMLQKARQTLEFYPCTSEEIDH |
| | | | EDITKDKTSTVEACLPLELTKNESCLNSRETSFITNGSCLASRKTSFMMALC |
| | | | LSSIYEDLKMYQVEFKTMNAKLLMDPKRQIFLDQNMLAVIDELMQALNFN |
| | | | SETVPQKSSLEEPDFYKTKIKLCILLHAFRIRAVTIDRVMSYLNAS |
| 65 | 22291 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGESKQEKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYPCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFYKTKIKLCILLHAFRIRAVTIDRVMSYLNAS |
| 66 | 22292 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGEKKDRVFTD |
| | | | KTSATVICRKNASISVRAQDRYYSSSWSEWASVPCSGGGGSGGGGSGGGG |
| | | | SNLPVATPDPGMFPCLHHSQNLLRAVSNMLQKARQTLEFYPCTSEEIDHED |
| | | | ITKDKTSTVEACLPLELTKNESCLNSRETSFITNGSCLASRKTSFMMALCLSS |
| | | | IYEDLKMYQVEFKTMNAKLLMDPKRQIFLDQNMLAVIDELMQALNFNSET |
| | | | VPQKSSLEEPDFYKTKIKLCILLHAFRIRAVTIDRVMSYLNAS |
| 67 | 22293 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQDQTDDRVFTD |
| | | | KTSATVICRKNASISVRAQDRYYSSSWSEWASVPCSGGGGSGGGGSGGGG |
| | | | SNLPVATPDPGMFPCLHHSQNLLRAVSNMLQKARQTLEFYPCTSEEIDHED |
| | | | ITKDKTSTVEACLPLELTKNESCLNSRETSFITNGSCLASRKTSFMMALCLSS |
| | | | IYEDLKMYQVEFKTMNAKLLMDPKRQIFLDQNMLAVIDELMQALNFNSET |
| | | | VPQKSSLEEPDFYKTKIKLCILLHAFRIRAVTIDRVMSYLNAS |
| 68 | 22294 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQDDSEDRVFTD |
| | | | KTSATVICRKNASISVRAQDRYYSSSWSEWASVPCSGGGGSGGGGSGGGG |
| | | | SNLPVATPDPGMFPCLHHSQNLLRAVSNMLQKARQTLEFYPCTSEEIDHED |
| | | | ITKDKTSTVEACLPLELTKNESCLNSRETSFITNGSCLASRKTSFMMALCLSS |
| | | | IYEDLKMYQVEFKTMNAKLLMDPKRQIFLDQNMLAVIDELMQALNFNSET |
| | | | VPQKSSLEEPDFYKTKIKLCILLHAFRIRAVTIDRVMSYLNAS |
| 69 | 22295 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVKDQTEDRVFTD |
| | | | KTSATVICRKNASISVRAQDRYYSSSWSEWASVPCSGGGGSGGGGSGGGG |
| | | | SNLPVATPDPGMFPCLHHSQNLLRAVSNMLQKARQTLEFYPCTSEEIDHED |
| | | | ITKDKTSTVEACLPLELTKNESCLNSRETSFITNGSCLASRKTSFMMALCLSS |
| | | | IYEDLKMYQVEFKTMNAKLLMDPKRQIFLDQNMLAVIDELMQALNFNSET |
| | | | VPQKSSLEEPDFYKTKIKLCILLHAFRIRAVTIDRVMSYLNAS |
| 70 | 22296 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGSEKDRVFTD |
| | | | KTSATVICRKNASISVRAQDRYYSSSWSEWASVPCSGGGGSGGGGSGGGG |
| | | | SNLPVATPDPGMFPCLHHSQNLLRAVSNMLQKARQTLEFYPCTSEEIDHED |
| | | | ITKDKTSTVEACLPLELTKNESCLNSRETSFITNGSCLASRKTSFMMALCLSS |
| | | | IYEDLKMYQVEFKTMNAKLLMDPKRQIFLDQNMLAVIDELMQALNFNSET |
| | | | VPQKSSLEEPDFYKTKIKLCILLHAFRIRAVTIDRVMSYLNAS |
| 71 | 22672 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSMSGRSANAGGGGSG |
| | | | GGGSGGGGSGGGGSKIDACKRGDVTVKPSHVILLGSTVNITCSLKPRQGCF |
| | | | HYSRRNKLILYKFDRRINFHHGHSLNSQVTGLPLGTTLFVCKLACINSDEIQI |
| | | | CGAEIFVGVAPEQPQNLSCIQKGEQGTVACTWERGRDTHLYTEYTLQLSGP |
| | | | KNLTWQKQCKDIYCDYLDFGINLTPESPESNFTAKVTAVNSLGSSSSLPSTF |
| | | | TFLDIVRPLPPWDIRIKFQKASVSRCTLYWRDEGLVLLNRLRYRPSNSRLW |
| | | | NMVNVTKAKGRHDLLDLKPFTEYEFQISSKLHLYKGSWSDWSESLRAQTP |
| | | | EEEP |
| 72 | 22735 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSGGGGSQVQLVESGGGVVQPGRSLRLSCAASGFTFSSYGMHWVRQA |
| | | | PGKGLEWVAFIRYDGSNKYYADSVKGRFTISRDNSKNTLYLQMNSLRAED |
| | | | TAVYYCKTHGSHDNWGQGTMVTVSSGGGGSMSGRSANAGGGGSGGGGS |
| | | | QSVLTQPPSVSGAPGQRVTISCSGSRSNIGSNTVKWYQQLPGTAPKLLIYYN |
| | | | DQRPSGVPDRFSGSKSGTSASLAITGLQAEDEADYYCQSYDRYTHPALLFG |
| | | | TGTKVTVL |
| 73 | 23360 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSGGGGSQSVLTQPPSVSGAPGQRVTISCSGSRSNIGSNTVKWYQQLPG |
| | | | TAPKLLIYYNDQRPSGVPDRFSGSKSGTSASLAITGLQAEDEADYYCQSYD |
| | | | RYTHPALLFGTGTKVTVLGGSGGGSGGGSGGGSGGGSGQVQLVESGGGV |
| | | | VQPGRSLRLSCAASGFTFSSYGMHWVRQAPGKGLEWVAFIRYDGSNKYY |
| | | | ADSVKGRFTISRDNSKNTLYLQMNSLRAEDTAVYYCKTHGSHDNWGQGT |
| | | | MVTVSS |
| 74 | 23361 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSGGGGSQSVLTQPPSVSGAPGQRVTISCSGSRSNIGSNTVKWYQQLPG |
| | | | TAPKLLIYYNDQRPSGVPDRFSGSKSGTSASLAITGLQAEDEADYYCQSYD |
| | | | RYTHPALLFGCGTKVTVLGGSGGGSGGGSGGGSGGGSGQVQLVESGGGV |
| | | | VQPGRSLRLSCAASGFTFSSYGMHWVRQAPGKCLEWVAFIRYDGSNKYY |
| | | | ADSVKGRFTISRDNSKNTLYLQMNSLRAEDTAVYYCKTHGSHDNWGQGT |
| | | | MVTVSS |
| 75 | 23363 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSGGGGSQVQLVESGGGVVQPGRSLRLSCAASGFTFSSYGMHWVRQA |
| | | | PGKGLEWVAFIRYDGSNKYYADSVKGRFTISRDNSKNTLYLQMNSLRAED |
| | | | TAVYYCKTHGSHDNWGQGTMVTVSSGGSGGGSMSGRSANAGGGSGGGS |
| | | | GGGSGQSVLTQPPSVSGAPGQRVTISCSGSRSNIGSNTVKWYQQLPGTAPK |
| | | | LLIYYNDQRPSGVPDRFSGSKSGTSASLAITGLQAEDEADYYCQSYDRYTH |
| | | | PALLFGTGTKVTVL |
| 76 | 23364 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSGGGGSQVQLVESGGGVVQPGRSLRLSCAASGFTFSSAGMHWVRQA |
| | | | PGKGLEWVAFIRYDGSNKYYADSVKGRFTISRDNSKNTLYLQMNSLRAED |
| | | | TAVYYCKTHGSHDNWGQGTMVTVSSGGSGGGSMSGRSANAGGGSGGGS |
| | | | GGGSGQSVLTQPPSVSGAPGQRVTISCSGSRSNIGSNTVKWYQQLPGTAPK |
| | | | LLIYYNDQRPSGVPDRFSGSKSGTSASLAITGLQAEDEADYYCQSYDRYTH |
| | | | PALLFGTGTKVTVL |
| 77 | 23512 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSGGGSGGGSG |
| | | | GGGSGGGGSQVQLVESGGGVVQPGRSLRLSCAASGFTFSSYGMHWVRQA |
| | | | PGKGLEWVAFIRYDGSNKYYADSVKGRFTISRDNSKNTLYLQMNSLRAED |
| | | | TAVYYCKTHGSHDNWGQGTMVTVSSGGGGSGGGSGGGSGGGGSGGGGS |
| | | | QSVLTQPPSVSGAPGQRVTISCSGSRSNIGSNTVKWYQQLPGTAPKLLIYYN |
| | | | DQRPSGVPDRFSGSKSGTSASLAITGLQAEDEADYYCQSYDRYTHPALLFG |
| | | | TGTKVTVL |
| 78 | 23513 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGSGGGSGGGGSG |
| | | | GGGSGGGGSGGGGSKIDACKRGDVTVKPSHVILLGSTVNITCSLKPRQGCF |
| | | | HYSRRNKLILYKFDRRINFHHGHSLNSQVTGLPLGTTLFVCKLACINSDEIQI |
| | | | CGAEIFVGVAPEQPQNLSCIQKGEQGTVACTWERGRDTHLYTEYTLQLSGP |
| | | | KNLTWQKQCKDIYCDYLDFGINLTPESPESNFTAKVTAVNSLGSSSSLPSTF |
| | | | TFLDIVRPLPPWDIRIKFQKASVSRCTLYWRDEGLVLLNRLRYRPSNSRLW |
| | | | NMVNVTKAKGRHDLLDLKPFTEYEFQISSKLHLYKGSWSDWSESLRAQTP |
| | | | EEEP |
| 79 | 23710 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGMSGRSANAGSADG |
| | | | GIWELKKDVYVVELDWYPDAPGEMVVLTCDTPEEDGITWTLDQSSEVLGS |
| | | | GKTLTIQVKEFGDAGQYTCHKGGEVLSHSLLLLHKKEDGIWSTDILKDQKE |
| | | | PKNKTFLRCEAKNYSGRFTCWWLTTISTDLTFSVKSSRGSSDPQGVTCGAA |
| | | | TLSAERVRGDNKEYEYSVECQEDSACPAAEESLPIEVMVDAVHKLKYENY |
| | | | TSSFFIRDIIKPDPPKNLQLKPLKNSRQVEVSWEYPDTWSTPHSYFSLTFCVQ |
| | | | VQGKSKREKKDRVFTDKTSATVICRKNASISVRAQDRYYSSSWSEWASVP |
| | | | CSGGGGSGGGGSGGGGSNLPVATPDPGMFPCLHHSQNLLRAVSNMLQKA |
| | | | RQTLEFYPCTSEEIDHEDITKDKTSTVEACLPLELTKNESCLNSRETSFITNGS |
| | | | CLASRKTSFMMALCLSSIYEDLKMYQVEFKTMNAKLLMDPKRQIFLDQNM |
| | | | LAVIDELMQALNFNSETVPQKSSLEEPDFYKTKIKLCILLHAFRIRAVTIDRV |
| | | | MSYLNAS |
| 80 | 23711 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGMSGRSANAGSADG |
| | | | GIWELKKDVYVVELDWYPDAPGEMVVLTCDTPEEDGITWTLDQSSEVLGS |
| | | | GKTLTIQVKEFGDAGQYTCHKGGEVLSHSLLLLHKKEDGIWSTDILKDQKE |
| | | | PKNKTFLRCEAKNYSGRFTCWWLTTISTDLTFSVKSSRGSSDPQGVTCGAA |
| | | | TLSAERVRGDNKEYEYSVECQEDSACPAAEESLPIEVMVDAVHKLKYENY |
| | | | TSSFFIRDIIKPDPPKNLQLKPLKNSRQVEVSWEYPDTWSTPHSYFSLTFCVQ |
| | | | VQGESKQEKKDRVFTDKTSATVICRKNASISVRAQDRYYSSSWSEWASVP |
| | | | CSGGGGSGGGGSGGGGSNLPVATPDPGMFPCLHHSQNLLRAVSNMLQKA |
| | | | RQTLEFYPCTSEEIDHEDITKDKTSTVEACLPLELTKNESCLNSRETSFITNGS |
| | | | CLASRKTSFMMALCLSSIYEDLKMYQVEFKTMNAKLLMDPKRQIFLDQNM |
| | | | LAVIDELMQALNFNSETVPQKSSLEEPDFYKTKIKLCILLHAFRIRAVTIDRV |
| | | | MSYLNAS |
| 81 | 24228 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYPCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFYKTKIKLCILLHAFRIRAVTIDRVMSYLNASGGGG |
| | | | SMSGRSANAGGGGSGGGGSGGGGSGGGGSEIVMTQSPATLSVSPGERATL |
| | | | SCRASQSISINLHWYQQKPGQAPRLLIYFASQSISGIPARFSGSGSGTEFTLTI |
| | | | SSLQSEDFAVYYCQQSNSFPLTFGGGTKVEIKGGSGGGSGGGSGGGSGGGS |
| | | | GQVQLVQSGAEVKKPGASVKVSCKASGYTFTDYYLHWVRQAPGQGLEW |
| | | | MGWIDPENGDTEYAPKFQGRVTMTTDTSTSTAYMELRSLRSDDTAVYYC |
| | | | NANKELRYFDVWGQGTMVTVSS |
| 82 | 24229 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSGGGGSQVQLVESGGGVVQPGRSLRLSCAASGFTFSSYGMHWVRQA |
| | | | PGKGLEWVAFIRYDGSNKYYADSVKGRFTISRDNSKNTLYLQMNSLRAED |
| | | | TAVYYCKTHGSHDNWGQGTMVTVSSGGGGSMSGRSANAGGGGSGGGGS |
| | | | QSVLTQPPSVSGAPGQRVTISCSGSRSNIGSNTVKWYQQLPGTAPKLLIYYN |
| | | | DQRPSGVPDRFSGSKSGTSASLAITGLQAEDEADYYCQSYDRYTHPALLFG |
| | | | TGTKVTVLGGGGSGGGGSGGGGSMSGRSANAGGGGSGGGGSGGGGSEIV |
| | | | MTQSPATLSVSPGERATLSCRASQSISINLHWYQQKPGQAPRLLIYFASQSIS |
| | | | GIPARFSGSGSGTEFTLTISSLQSEDFAVYYCQQSNSFPLTFGGGTKVEIKGG |
| | | | SGGGSGGGSGGGSGGGSGQVQLVQSGAEVKKPGASVKVSCKASGYTFTD |
| | | | YYLHWVRQAPGQGLEWMGWIDPENGDTEYAPKFQGRVTMTTDTSTSTAY |
| | | | MELRSLRSDDTAVYYCNANKELRYFDVWGQGTMVTVSS |
| 83 | 24230 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSGGGGSQVQLVESGGGVVQPGRSLRLSCAASGFTFSSYGMHWVRQA |
| | | | PGKGLEWVAFIRYDGSNKYYADSVKGRFTISRDNSKNTLYLQMNSLRAED |
| | | | TAVYYCKTHGSHDNWGQGTMVTVSSGGGGSMSGRSANAGGGGSGGGGS |
| | | | QSVLTQPPSVSGAPGQRVTISCSGSRSNIGSNTVKWYQQLPGTAPKLLIYYN |
| | | | DQRPSGVPDRFSGSKSGTSASLAITGLQAEDEADYYCQSYDRYTHPALLFG |
| | | | TGTKVTVLGGGGSGGGGSGGGGSMSGRSANAGGGGSGGGGSGGGGSQV |
| | | | QLVQSGAEVKKPGASVKVSCKASGYTFTDYYLHWVRQAPGQGLEWMGW |
| | | | IDPENGDTEYAPKFQGRVTMTTDTSTSTAYMELRSLRSDDTAVYYCNANK |
| | | | ELRYFDVWGQGTMVTVSSGGSGGGSGGGSGGGSGGGSGEIVMTQSPATLS |
| | | | VSPGERATLSCRASQSISINLHWYQQKPGQAPRLLIYFASQSISGIPARFSGS |
| | | | GSGTEFTLTISSLQSEDFAVYYCQQSNSFPLTFGGGTKVEIK |
| 84 | 24231 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYPCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFYKTKIKLCILLHAFRIRAVTIDRVMSYLNASGGGG |
| | | | SMSGRSANAGGGGSGGGGSGGGGSGGGGSGGGGSKIDACKRGDVTVKPS |
| | | | HVILLGSTVNITCSLKPRQGCFHYSRRNKLILYKFDRRINFHHGHSLNSQVT |
| | | | GLPLGTTLFVCKLACINSDEIQICGAEIFVGVAPEQPQNLSCIQKGEQGTVAC |
| | | | TWERGRDTHLYTEYTLQLSGPKNLTWQKQCKDIYCDYLDFGINLTPESPES |
| | | | NFTAKVTAVNSLGSSSSLPSTFTFLDIVRPLPPWDIRIKFQKASVSRCTLYWR |
| | | | DEGLVLLNRLRYRPSNSRLWNMVNVTKAKGRHDLLDLKPFTEYEFQISSK |
| | | | LHLYKGSWSDWSESLRAQTPEEEP |
| 85 | 24232 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSGGGGSMSGR |
| | | | SANAGGGGSGGGGSGGGGSCRTSECCFQDPPYPDADSGSASGPRDLRCYRI |
| | | | SSDRYECSWQYEGPTAGVSHFLRCCLSSGRCCYFAAGSATRLQFSDQAGV |
| | | | SVLYTVTLWVESWARNQTEKSPEVTLQLYNSVKYEPPLGDIKVSKLAGQL |
| | | | RMEWETPDNQVGAEVQFRHRTPSSPWKLGDCGPQDDDTESCLCPLEMNV |
| | | | AQEFQLRRRQLGSQGSSWSKWSSPVCVPPENPPQPQ |
| 86 | 24233 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSMSGRSANAGGGGSG |
| | | | GGGSGGGGSGGGGSKIDACKRGDVTVKPSHVILLGSTVNITCSLKPRQGCF |
| | | | HYSRRNKLILYKFDRRINFHHGHSLNSQVTGLPLGTTLFVCKLACINSDEIQI |
| | | | CGAEIFVGVAPGGGGSGGGGSGGGGSMSGRSANAGGGGSGGGGSGGGGS |
| | | | CRTSECCFQDPPYPDADSGSASGPRDLRCYRISSDRYECSWQYEGPTAGVS |
| | | | HFLRCCLSSGRCCYFAAGSATRLQFSDQAGVSVLYTVTLWVESWARNQTE |
| | | | KSPEVTLQLYNSVKYEPPLGDIKVSKLAGQLRMEWETPDNQVGAEVQFRH |
| | | | RTPSSPWKLGDCGPQDDDTESCLCPLEMNVAQEFQLRRRQLGSQGSSWSK |
| | | | WSSPVCVPPENPPQPQ |
| 87 | 24235 | Full AA | KIDACKRGDVTVKPSHVILLGSTVNITCSLKPRQGCFHYSRRNKLILYKFDR |
| | | | RINFHHGHSLNSQVTGLPLGTTLFVCKLACINSDEIQICGAEIFVGVAPGGG |
| | | | GSGGGGSMSGRSANAGGGGSRNLPVATPDPGMFPCLHHSQNLLRAVSNM |
| | | | LQKARQTLEFYPCTSEEIDHEDITKDKTSTVEACLPLELTKNESCLNSRETSF |
| | | | ITNGSCLASRKTSFMMALCLSSIYEDLKMYQVEFKTMNAKLLMDPKRQIFL |
| | | | DQNMLAVIDELMQALNFNSETVPQKSSLEEPDFYKTKIKLCILLHAFRIRAV |
| | | | TIDRVMSYLNASGGGGSGGGGSEPKSSDKTHTCPPCPAPEAAGGPSVFLFPP |
| | | | KPKDTLMISRTPEVTCVVVSVSHEDPEVKFNWYVDGVEVHNAKTKPREEQ |
| | | | YNSTYRVVSVLTVLHQDWLNGKEYKCKVSNKALPAPIEKTISKAKGQPRE |
| | | | PQVYVYPPSRDELTKNQVSLTCLVKGFYPSDIAVEWESNGQPENNYKTTPP |
| | | | VLDSDGSFALVSKLTVDKSRWQQGNVFSCSVMHEALHNHYTQKSLSLSPG |
| 88 | 24236 | Full AA | CRTSECCFQDPPYPDADSGSASGPRDLRCYRISSDRYECSWQYEGPTAGVS |
| | | | HFLRCCLSSGRCCYFAAGSATRLQFSDQAGVSVLYTVTLWVESWARNQTE |
| | | | KSPEVTLQLYNSVKYEPPLGDIKVSKLAGQLRMEWETPDNQVGAEVQFRH |
| | | | RTPSSPWKLGDCGPQDDDTESCLCPLEMNVAQEFQLRRRQLGSQGSSWSK |
| | | | WSSPVCVPPENPPQPQGGGGSGGGGSGGGGSGGGGSMSGRSANAEPKSSD |
| | | | KTHTCPPCPAPEAAGGPSVFLFPPKPKDTLMISRTPEVTCVVVSVSHEDPEV |
| | | | KFNWYVDGVEVHNAKTKPREEQYNSTYRVVSVLTVLHQDWLNGKEYKC |
| | | | KVSNKALPAPIEKTISKAKGQPREPQVYVLPPSRDELTKNQVSLLCLVKGFY |
| | | | PSDIAVEWESNGQPENNYLTWPPVLDSDGSFFLYSKLTVDKSRWQQGNVF |
| | | | SCSVMHEALHNHYTQKSLSLSPG |
| 89 | 24246 | Full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGGGGSMSGRSANAGGGGSG |
| | | | GGGSGGGGSGGGGSKIDACKRGDVTVKPSHVILLGSTVNITCSLKPRQGCF |
| | | | HYSRRNKLILYKFDRRINFHHGHSLNSQVTGLPLGTTLFVCKLACINSDEIQI |
| | | | CGAEIFVGVAPGGGGSGGGGSMSGRSANAGGGGSRNLPVATPDPGMFPCL |
| | | | HHSQNLLRAVSNMLQKARQTLEFYPCTSEEIDHEDITKDKTSTVEACLPLEL |
| | | | TKNESCLNSRETSFITNGSCLASRKTSFMMALCLSSIYEDLKMYQVEFKTM |
| | | | NAKLLMDPKRQIFLDQNMLAVIDELMQALNFNSETVPQKSSLEEPDFYKTK |
| | | | IKLCILLHAFRIRAVTIDRVMSYLNAS |
| 90 | 12153 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAACCACAGGTCTACGTGCTGCCTCCATCCCGGGACGAGCTGACAAAG |
| | | | AACCAGGTCTCTCTGCTGTGCCTGGTGAAAGGCTTCTATCCATCAGATA |
| | | | TTGCTGTGGAGTGGGAAAGCAATGGGCAGCCCGAGAACAATTACCTGA |
| | | | CTTGGCCCCCTGTGCTGGACTCTGATGGGAGTTTCTTTCTGTATTCTAAG |
| | | | CTGACCGTGGATAAAAGTAGGTGGCAGCAGGGAAATGTCTTTAGTTGTT |
| | | | CAGTGATGCATGAAGCCCTGCATAACCACTACACCCAGAAAAGCCTGT |
| | | | CCCTGTCCCCCGGA |
| 91 | 12155 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCACAGGTCTACGTGTATCCTCCAAGCCGGGACGAGCTGACAAAGA |
| | | | ACCAGGTCTCCCTGACTTGTCTGGTGAAAGGGTTTTACCCTAGTGATAT |
| | | | CGCTGTGGAGTGGGAATCAAATGGACAGCCAGAGAACAATTATAAGAC |
| | | | TACCCCCCCTGTGCTGGACAGTGATGGGTCATTCGCACTGGTCTCCAAG |
| | | | CTGACAGTGGACAAATCTCGGTGGCAGCAGGGAAATGTCTTTTCATGTA |
| | | | GCGTGATGCATGAAGCACTGCACAACCATTACACCCAGAAGTCACTGTC |
| | | | ACTGTCACCAGGA |
| 92 | 12933 | Full nt | GACATCCAGATGACACAGTCTCCTAGCTCCCTGTCCGCCTCTGTGGGCG |
| | | | ATAGAGTGACCATCACATGCAGCGCCTCTAGCTCCGTGTCCTACATGCA |
| | | | CTGGTATCAGCAGAAGAGCGGCAAGGCCCCAAAGCTGCTGATCTACGA |
| | | | CACCAGCAAGCTGGCCTCCGGAGTGCCATCTAGGTTCAGCGGCTCCGGC |
| | | | TCTGGCACCGACTTTACCCTGACAATCTCTAGCCTGCAGCCTGAGGATT |
| | | | TCGCCACATACTATTGTCAGCAGTGGTCCGGCTATCCACTGACCTTTGG |
| | | | CCAGGGCACAAAGCTGGAGATCAAGCGCACAGTGGCGGCGCCCAGTGT |
| | | | CTTCATTTTTCCCCCTAGCGACGAACAGCTGAAGTCTGGGACAGCCAGT |
| | | | GTGGTCTGTCTGCTGAACAACTTCTACCCTAGAGAGGCTAAAGTGCAGT |
| | | | GGAAGGTCGATAACGCACTGCAGTCCGGAAATTCTCAGGAGAGTGTGA |
| | | | CTGAACAGGACTCAAAAGATAGCACCTATTCCCTGTCAAGCACACTGAC |
| | | | TCTGAGCAAGGCCGACTACGAGAAGCATAAAGTGTATGCTTGTGAAGT |
| | | | CACCCACCAGGGGCTGAGTTCACCAGTCACAAAATCATTCAACAGAGG |
| | | | GGAGTGC |
| 93 | 17572 | Full nt | CAAGTGGGAGCCTGCCCTAGCGGCAAGAAGGCCCGCGAGATCGACGAG |
| | | | TCCCTGATCTTCTACAAGAAGTGGGAGCTGGAGGCATGCGTGGACGCC |
| | | | GCCCTGCTGGCCACACAGATGGATAGGGTGAACGCCATCCCTTTTACCT |
| | | | ATGAGCAGCTGGATGTGCTGAAGCACAAGCTGCCACAGGGACAGGGAG |
| | | | GAGGAGGCTCCGGAGGAGGCGGCAACTCTCCACTGAGCGGGCGGAGCG |
| | | | ACAATCACCAGGGACAGTCTGGACAGGGAGGACAGGTGCAGCTGGTGC |
| | | | AGAGCGGAGCCGAGGTGAAGAAGCCTGGGGCCAGCGTGAAGGTGTCTT |
| | | | GCAAGGCCTCTGGCTACAGCTTCACAGGCTATACCATGAATTGGGTGCG |
| | | | GCAGGCCCCAGGACAGGGACTGGAGTGGATGGGCCTGATCACACCCTA |
| | | | CAACGGGGCCAGCTCCTATAATCAGAAGTTTCGGGGCAAGGCCACCAT |
| | | | GACAGTGGACACCAGCACATCCACCGTGTACATGGAGCTGTCTAGCCTG |
| | | | AGAAGCGAGGATACCGCCGTGTACTATTGTGCCAGAGGCGGCTACGAC |
| | | | GGCAGAGGCTTTGATTATTGGGGCCAGGGCACACTGGTGACCGTGTCCT |
| | | | CTGCTAGCACTAAGGGGCCTTCCGTGTTTCCACTGGCTCCCTCTAGTAA |
| | | | ATCCACCTCTGGAGGCACAGCTGCACTGGGATGTCTGGTGAAGGATTAC |
| | | | TTCCCTGAACCAGTCACAGTGAGTTGGAACTCAGGGGCTCTGACAAGTG |
| | | | GAGTCCATACTTTTCCCGCAGTGCTGCAGTCAAGCGGACTGTACTCCCT |
| | | | GTCCTCTGTGGTCACCGTGCCTAGTTCAAGCCTGGGCACCCAGACATAT |
| | | | ATCTGCAACGTGAATCACAAGCCATCAAATACAAAAGTCGACAAGAAA |
| | | | GTGGAGCCCAAGAGCTGTGATAAAACTCATACCTGCCCACCTTGTCCGG |
| | | | CGCCAGAGGCAGCAGGAGGACCAAGCGTGTTCCTGTTTCCACCCAAGC |
| | | | CCAAAGACACCCTGATGATTAGCCGAACCCCTGAAGTCACATGCGTGGT |
| | | | CGTGTCCGTGTCTCACGAGGACCCAGAAGTCAAGTTCAACTGGTACGTG |
| | | | GATGGCGTCGAGGTGCATAATGCCAAGACAAAACCCCGGGAGGAACAG |
| | | | TACAACAGCACCTATAGAGTCGTGTCCGTCCTGACAGTGCTGCACCAGG |
| | | | ATTGGCTGAACGGCAAGGAATATAAGTGCAAAGTGTCCAATAAGGCCC |
| | | | TGCCCGCTCCTATCGAGAAAACCATTTCTAAGGCAAAAGGCCAGCCTCG |
| | | | CGAACCACAGGTCTACGTGCTGCCTCCATCCCGGGACGAGCTGACAAA |
| | | | GAACCAGGTCTCTCTGCTGTGCCTGGTGAAAGGCTTCTATCCATCAGAT |
| | | | ATTGCTGTGGAGTGGGAAAGCAATGGGCAGCCCGAGAACAATTACCTG |
| | | | ACTTGGCCCCCTGTGCTGGACTCTGATGGGAGTTTCTTTCTGTATTCTAA |
| | | | GCTGACCGTGGATAAAAGTAGGTGGCAGCAGGGAAATGTCTTTAGTTG |
| | | | TTCAGTGATGCATGAAGCCCTGCATAACCACTACACCCAGAAAAGCCTG |
| | | | TCCCTGTCCCCCGGA |
| 94 | 17573 | Full nt | CAAGTGGGAGCCTGCCCTAGCGGCAAGAAGGCCCGCGAGATCGACGAG |
| | | | TCCCTGATCTTCTACAAGAAGTGGGAGCTGGAGGCATGCGTGGACGCC |
| | | | GCCCTGCTGGCCACACAGATGGATAGGGTGAACGCCATCCCTTTTACCT |
| | | | ATGAGCAGCTGGATGTGCTGAAGCACAAGCTGCCACAGGGACAGGGAG |
| | | | GAGGAGGCTCCGGAGGAGGCGGCAACTCTCCAACCAGCGGGCGGAGCG |
| | | | CAAATCCCCAGGGACAGTCTGGACAGGGAGGACAGGTGCAGCTGGTGC |
| | | | AGAGCGGAGCCGAGGTGAAGAAGCCTGGGGCCAGCGTGAAGGTGTCTT |
| | | | GCAAGGCCTCTGGCTACAGCTTCACAGGCTATACCATGAATTGGGTGCG |
| | | | GCAGGCCCCAGGACAGGGACTGGAGTGGATGGGCCTGATCACACCCTA |
| | | | CAACGGGGCCAGCTCCTATAATCAGAAGTTTCGGGGCAAGGCCACCAT |
| | | | GACAGTGGACACCAGCACATCCACCGTGTACATGGAGCTGTCTAGCCTG |
| | | | AGAAGCGAGGATACCGCCGTGTACTATTGTGCCAGAGGCGGCTACGAC |
| | | | GGCAGAGGCTTTGATTATTGGGGCCAGGGCACACTGGTGACCGTGTCCT |
| | | | CTGCTAGCACTAAGGGGCCTTCCGTGTTTCCACTGGCTCCCTCTAGTAA |
| | | | ATCCACCTCTGGAGGCACAGCTGCACTGGGATGTCTGGTGAAGGATTAC |
| | | | TTCCCTGAACCAGTCACAGTGAGTTGGAACTCAGGGGCTCTGACAAGTG |
| | | | GAGTCCATACTTTTCCCGCAGTGCTGCAGTCAAGCGGACTGTACTCCCT |
| | | | GTCCTCTGTGGTCACCGTGCCTAGTTCAAGCCTGGGCACCCAGACATAT |
| | | | ATCTGCAACGTGAATCACAAGCCATCAAATACAAAAGTCGACAAGAAA |
| | | | GTGGAGCCCAAGAGCTGTGATAAAACTCATACCTGCCCACCTTGTCCGG |
| | | | CGCCAGAGGCAGCAGGAGGACCAAGCGTGTTCCTGTTTCCACCCAAGC |
| | | | CCAAAGACACCCTGATGATTAGCCGAACCCCTGAAGTCACATGCGTGGT |
| | | | CGTGTCCGTGTCTCACGAGGACCCAGAAGTCAAGTTCAACTGGTACGTG |
| | | | GATGGCGTCGAGGTGCATAATGCCAAGACAAAACCCCGGGAGGAACAG |
| | | | TACAACAGCACCTATAGAGTCGTGTCCGTCCTGACAGTGCTGCACCAGG |
| | | | ATTGGCTGAACGGCAAGGAATATAAGTGCAAAGTGTCCAATAAGGCCC |
| | | | TGCCCGCTCCTATCGAGAAAACCATTTCTAAGGCAAAAGGCCAGCCTCG |
| | | | CGAACCACAGGTCTACGTGCTGCCTCCATCCCGGGACGAGCTGACAAA |
| | | | GAACCAGGTCTCTCTGCTGTGCCTGGTGAAAGGCTTCTATCCATCAGAT |
| | | | ATTGCTGTGGAGTGGGAAAGCAATGGGCAGCCCGAGAACAATTACCTG |
| | | | ACTTGGCCCCCTGTGCTGGACTCTGATGGGAGTTTCTTTCTGTATTCTAA |
| | | | GCTGACCGTGGATAAAAGTAGGTGGCAGCAGGGAAATGTCTTTAGTTG |
| | | | TTCAGTGATGCATGAAGCCCTGCATAACCACTACACCCAGAAAAGCCTG |
| | | | TCCCTGTCCCCCGGA |
| 95 | 17577 | Full nt | CAAGTGGGAGCCTGCCCTAGCGGCAAGAAGGCCCGCGAGATCGACGAG |
| | | | TCCCTGATCTTCTACAAGAAGTGGGAGCTGGAGGCATGCGTGGACGCC |
| | | | GCCCTGCTGGCCACACAGATGGATAGGGTGAACGCCATCCCTTTTACCT |
| | | | ATGAGCAGCTGGATGTGCTGAAGCACAAGCTGCCACAGGGACAGGGAG |
| | | | GAGGAGGCTCCGGAGGAGGCGGCAACTCTCCAGGGAGCGGGCGGAGC |
| | | | GCACAGGTGCAGGGACAGTCTGGACAGGGAGGACAGGTGCAGCTGGTG |
| | | | CAGAGCGGAGCCGAGGTGAAGAAGCCTGGGGCCAGCGTGAAGGTGTCT |
| | | | TGCAAGGCCTCTGGCTACAGCTTCACAGGCTATACCATGAATTGGGTGC |
| | | | GGCAGGCCCCAGGACAGGGACTGGAGTGGATGGGCCTGATCACACCCT |
| | | | ACAACGGGGCCAGCTCCTATAATCAGAAGTTTCGGGGCAAGGCCACCA |
| | | | TGACAGTGGACACCAGCACATCCACCGTGTACATGGAGCTGTCTAGCCT |
| | | | GAGAAGCGAGGATACCGCCGTGTACTATTGTGCCAGAGGCGGCTACGA |
| | | | CGGCAGAGGCTTTGATTATTGGGGCCAGGGCACACTGGTGACCGTGTCC |
| | | | TCTGCTAGCACTAAGGGGCCTTCCGTGTTTCCACTGGCTCCCTCTAGTAA |
| | | | ATCCACCTCTGGAGGCACAGCTGCACTGGGATGTCTGGTGAAGGATTAC |
| | | | TTCCCTGAACCAGTCACAGTGAGTTGGAACTCAGGGGCTCTGACAAGTG |
| | | | GAGTCCATACTTTTCCCGCAGTGCTGCAGTCAAGCGGACTGTACTCCCT |
| | | | GTCCTCTGTGGTCACCGTGCCTAGTTCAAGCCTGGGCACCCAGACATAT |
| | | | ATCTGCAACGTGAATCACAAGCCATCAAATACAAAAGTCGACAAGAAA |
| | | | GTGGAGCCCAAGAGCTGTGATAAAACTCATACCTGCCCACCTTGTCCGG |
| | | | CGCCAGAGGCAGCAGGAGGACCAAGCGTGTTCCTGTTTCCACCCAAGC |
| | | | CCAAAGACACCCTGATGATTAGCCGAACCCCTGAAGTCACATGCGTGGT |
| | | | CGTGTCCGTGTCTCACGAGGACCCAGAAGTCAAGTTCAACTGGTACGTG |
| | | | GATGGCGTCGAGGTGCATAATGCCAAGACAAAACCCCGGGAGGAACAG |
| | | | TACAACAGCACCTATAGAGTCGTGTCCGTCCTGACAGTGCTGCACCAGG |
| | | | ATTGGCTGAACGGCAAGGAATATAAGTGCAAAGTGTCCAATAAGGCCC |
| | | | TGCCCGCTCCTATCGAGAAAACCATTTCTAAGGCAAAAGGCCAGCCTCG |
| | | | CGAACCACAGGTCTACGTGCTGCCTCCATCCCGGGACGAGCTGACAAA |
| | | | GAACCAGGTCTCTCTGCTGTGCCTGGTGAAAGGCTTCTATCCATCAGAT |
| | | | ATTGCTGTGGAGTGGGAAAGCAATGGGCAGCCCGAGAACAATTACCTG |
| | | | ACTTGGCCCCCTGTGCTGGACTCTGATGGGAGTTTCTTTCTGTATTCTAA |
| | | | GCTGACCGTGGATAAAAGTAGGTGGCAGCAGGGAAATGTCTTTAGTTG |
| | | | TTCAGTGATGCATGAAGCCCTGCATAACCACTACACCCAGAAAAGCCTG |
| | | | TCCCTGTCCCCCGGA |
| 96 | 17578 | Full nt | CAAGTGGGAGCCTGCCCTAGCGGCAAGAAGGCCCGCGAGATCGACGAG |
| | | | TCCCTGATCTTCTACAAGAAGTGGGAGCTGGAGGCATGCGTGGACGCC |
| | | | GCCCTGCTGGCCACACAGATGGATAGGGTGAACGCCATCCCTTTTACCT |
| | | | ATGAGCAGCTGGATGTGCTGAAGCACAAGCTGCCACAGGGACAGGGAG |
| | | | GAGGAGGCTCCGGAGGAGGCGGCAACTCTCCAGGGAGCAGCCGGAAC |
| | | | GCAGACGTGCAGGGACAGTCTGGACAGGGAGGACAGGTGCAGCTGGTG |
| | | | CAGAGCGGAGCCGAGGTGAAGAAGCCTGGGGCCAGCGTGAAGGTGTCT |
| | | | TGCAAGGCCTCTGGCTACAGCTTCACAGGCTATACCATGAATTGGGTGC |
| | | | GGCAGGCCCCAGGACAGGGACTGGAGTGGATGGGCCTGATCACACCCT |
| | | | ACAACGGGGCCAGCTCCTATAATCAGAAGTTTCGGGGCAAGGCCACCA |
| | | | TGACAGTGGACACCAGCACATCCACCGTGTACATGGAGCTGTCTAGCCT |
| | | | GAGAAGCGAGGATACCGCCGTGTACTATTGTGCCAGAGGCGGCTACGA |
| | | | CGGCAGAGGCTTTGATTATTGGGGCCAGGGCACACTGGTGACCGTGTCC |
| | | | TCTGCTAGCACTAAGGGGCCTTCCGTGTTTCCACTGGCTCCCTCTAGTAA |
| | | | ATCCACCTCTGGAGGCACAGCTGCACTGGGATGTCTGGTGAAGGATTAC |
| | | | TTCCCTGAACCAGTCACAGTGAGTTGGAACTCAGGGGCTCTGACAAGTG |
| | | | GAGTCCATACTTTTCCCGCAGTGCTGCAGTCAAGCGGACTGTACTCCCT |
| | | | GTCCTCTGTGGTCACCGTGCCTAGTTCAAGCCTGGGCACCCAGACATAT |
| | | | ATCTGCAACGTGAATCACAAGCCATCAAATACAAAAGTCGACAAGAAA |
| | | | GTGGAGCCCAAGAGCTGTGATAAAACTCATACCTGCCCACCTTGTCCGG |
| | | | CGCCAGAGGCAGCAGGAGGACCAAGCGTGTTCCTGTTTCCACCCAAGC |
| | | | CCAAAGACACCCTGATGATTAGCCGAACCCCTGAAGTCACATGCGTGGT |
| | | | CGTGTCCGTGTCTCACGAGGACCCAGAAGTCAAGTTCAACTGGTACGTG |
| | | | GATGGCGTCGAGGTGCATAATGCCAAGACAAAACCCCGGGAGGAACAG |
| | | | TACAACAGCACCTATAGAGTCGTGTCCGTCCTGACAGTGCTGCACCAGG |
| | | | ATTGGCTGAACGGCAAGGAATATAAGTGCAAAGTGTCCAATAAGGCCC |
| | | | TGCCCGCTCCTATCGAGAAAACCATTTCTAAGGCAAAAGGCCAGCCTCG |
| | | | CGAACCACAGGTCTACGTGCTGCCTCCATCCCGGGACGAGCTGACAAA |
| | | | GAACCAGGTCTCTCTGCTGTGCCTGGTGAAAGGCTTCTATCCATCAGAT |
| | | | ATTGCTGTGGAGTGGGAAAGCAATGGGCAGCCCGAGAACAATTACCTG |
| | | | ACTTGGCCCCCTGTGCTGGACTCTGATGGGAGTTTCTTTCTGTATTCTAA |
| | | | GCTGACCGTGGATAAAAGTAGGTGGCAGCAGGGAAATGTCTTTAGTTG |
| | | | TTCAGTGATGCATGAAGCCCTGCATAACCACTACACCCAGAAAAGCCTG |
| | | | TCCCTGTCCCCCGGA |
| 97 | 17580 | Full nt | CAAGTGGGAGCCTGCCCTAGCGGCAAGAAGGCCCGCGAGATCGACGAG |
| | | | TCCCTGATCTTCTACAAGAAGTGGGAGCTGGAGGCATGCGTGGACGCC |
| | | | GCCCTGCTGGCCACACAGATGGATAGGGTGAACGCCATCCCTTTTACCT |
| | | | ATGAGCAGCTGGATGTGCTGAAGCACAAGCTGCCACAGGGACAGGGAG |
| | | | GAGGAGGCTCCGGAGGAGGCGGCAACTCTCCAGGGACCGCCCGGAGCG |
| | | | ACAATGTGCAGGGACAGTCTGGACAGGGAGGACAGGTGCAGCTGGTGC |
| | | | AGAGCGGAGCCGAGGTGAAGAAGCCTGGGGCCAGCGTGAAGGTGTCTT |
| | | | GCAAGGCCTCTGGCTACAGCTTCACAGGCTATACCATGAATTGGGTGCG |
| | | | GCAGGCCCCAGGACAGGGACTGGAGTGGATGGGCCTGATCACACCCTA |
| | | | CAACGGGGCCAGCTCCTATAATCAGAAGTTTCGGGGCAAGGCCACCAT |
| | | | GACAGTGGACACCAGCACATCCACCGTGTACATGGAGCTGTCTAGCCTG |
| | | | AGAAGCGAGGATACCGCCGTGTACTATTGTGCCAGAGGCGGCTACGAC |
| | | | GGCAGAGGCTTTGATTATTGGGGCCAGGGCACACTGGTGACCGTGTCCT |
| | | | CTGCTAGCACTAAGGGGCCTTCCGTGTTTCCACTGGCTCCCTCTAGTAA |
| | | | ATCCACCTCTGGAGGCACAGCTGCACTGGGATGTCTGGTGAAGGATTAC |
| | | | TTCCCTGAACCAGTCACAGTGAGTTGGAACTCAGGGGCTCTGACAAGTG |
| | | | GAGTCCATACTTTTCCCGCAGTGCTGCAGTCAAGCGGACTGTACTCCCT |
| | | | GTCCTCTGTGGTCACCGTGCCTAGTTCAAGCCTGGGCACCCAGACATAT |
| | | | ATCTGCAACGTGAATCACAAGCCATCAAATACAAAAGTCGACAAGAAA |
| | | | GTGGAGCCCAAGAGCTGTGATAAAACTCATACCTGCCCACCTTGTCCGG |
| | | | CGCCAGAGGCAGCAGGAGGACCAAGCGTGTTCCTGTTTCCACCCAAGC |
| | | | CCAAAGACACCCTGATGATTAGCCGAACCCCTGAAGTCACATGCGTGGT |
| | | | CGTGTCCGTGTCTCACGAGGACCCAGAAGTCAAGTTCAACTGGTACGTG |
| | | | GATGGCGTCGAGGTGCATAATGCCAAGACAAAACCCCGGGAGGAACAG |
| | | | TACAACAGCACCTATAGAGTCGTGTCCGTCCTGACAGTGCTGCACCAGG |
| | | | ATTGGCTGAACGGCAAGGAATATAAGTGCAAAGTGTCCAATAAGGCCC |
| | | | TGCCCGCTCCTATCGAGAAAACCATTTCTAAGGCAAAAGGCCAGCCTCG |
| | | | CGAACCACAGGTCTACGTGCTGCCTCCATCCCGGGACGAGCTGACAAA |
| | | | GAACCAGGTCTCTCTGCTGTGCCTGGTGAAAGGCTTCTATCCATCAGAT |
| | | | ATTGCTGTGGAGTGGGAAAGCAATGGGCAGCCCGAGAACAATTACCTG |
| | | | ACTTGGCCCCCTGTGCTGGACTCTGATGGGAGTTTCTTTCTGTATTCTAA |
| | | | GCTGACCGTGGATAAAAGTAGGTGGCAGCAGGGAAATGTCTTTAGTTG |
| | | | TTCAGTGATGCATGAAGCCCTGCATAACCACTACACCCAGAAAAGCCTG |
| | | | TCCCTGTCCCCCGGA |
| 98 | 17584 | Full nt | CAAGTGGGAGCCTGCCCTAGCGGCAAGAAGGCCCGCGAGATCGACGAG |
| | | | TCCCTGATCTTCTACAAGAAGTGGGAGCTGGAGGCATGCGTGGACGCC |
| | | | GCCCTGCTGGCCACACAGATGGATAGGGTGAACGCCATCCCTTTTACCT |
| | | | ATGAGCAGCTGGATGTGCTGAAGCACAAGCTGCCACAGGGACAGGGAG |
| | | | GAGGAGGCTCCGGAGGAGGCGGCAACTCTCCAGGGGGCGGGCGGGTG |
| | | | AACAATGTGCAGGGACAGTCTGGACAGGGAGGACAGGTGCAGCTGGTG |
| | | | CAGAGCGGAGCCGAGGTGAAGAAGCCTGGGGCCAGCGTGAAGGTGTCT |
| | | | TGCAAGGCCTCTGGCTACAGCTTCACAGGCTATACCATGAATTGGGTGC |
| | | | GGCAGGCCCCAGGACAGGGACTGGAGTGGATGGGCCTGATCACACCCT |
| | | | ACAACGGGGCCAGCTCCTATAATCAGAAGTTTCGGGGCAAGGCCACCA |
| | | | TGACAGTGGACACCAGCACATCCACCGTGTACATGGAGCTGTCTAGCCT |
| | | | GAGAAGCGAGGATACCGCCGTGTACTATTGTGCCAGAGGCGGCTACGA |
| | | | CGGCAGAGGCTTTGATTATTGGGGCCAGGGCACACTGGTGACCGTGTCC |
| | | | TCTGCTAGCACTAAGGGGCCTTCCGTGTTTCCACTGGCTCCCTCTAGTAA |
| | | | ATCCACCTCTGGAGGCACAGCTGCACTGGGATGTCTGGTGAAGGATTAC |
| | | | TTCCCTGAACCAGTCACAGTGAGTTGGAACTCAGGGGCTCTGACAAGTG |
| | | | GAGTCCATACTTTTCCCGCAGTGCTGCAGTCAAGCGGACTGTACTCCCT |
| | | | GTCCTCTGTGGTCACCGTGCCTAGTTCAAGCCTGGGCACCCAGACATAT |
| | | | ATCTGCAACGTGAATCACAAGCCATCAAATACAAAAGTCGACAAGAAA |
| | | | GTGGAGCCCAAGAGCTGTGATAAAACTCATACCTGCCCACCTTGTCCGG |
| | | | CGCCAGAGGCAGCAGGAGGACCAAGCGTGTTCCTGTTTCCACCCAAGC |
| | | | CCAAAGACACCCTGATGATTAGCCGAACCCCTGAAGTCACATGCGTGGT |
| | | | CGTGTCCGTGTCTCACGAGGACCCAGAAGTCAAGTTCAACTGGTACGTG |
| | | | GATGGCGTCGAGGTGCATAATGCCAAGACAAAACCCCGGGAGGAACAG |
| | | | TACAACAGCACCTATAGAGTCGTGTCCGTCCTGACAGTGCTGCACCAGG |
| | | | ATTGGCTGAACGGCAAGGAATATAAGTGCAAAGTGTCCAATAAGGCCC |
| | | | TGCCCGCTCCTATCGAGAAAACCATTTCTAAGGCAAAAGGCCAGCCTCG |
| | | | CGAACCACAGGTCTACGTGCTGCCTCCATCCCGGGACGAGCTGACAAA |
| | | | GAACCAGGTCTCTCTGCTGTGCCTGGTGAAAGGCTTCTATCCATCAGAT |
| | | | ATTGCTGTGGAGTGGGAAAGCAATGGGCAGCCCGAGAACAATTACCTG |
| | | | ACTTGGCCCCCTGTGCTGGACTCTGATGGGAGTTTCTTTCTGTATTCTAA |
| | | | GCTGACCGTGGATAAAAGTAGGTGGCAGCAGGGAAATGTCTTTAGTTG |
| | | | TTCAGTGATGCATGAAGCCCTGCATAACCACTACACCCAGAAAAGCCTG |
| | | | TCCCTGTCCCCCGGA |
| 99 | 17586 | Full nt | CAAGTGGGAGCCTGCCCTTCTGGCAAGAAGGCCCGCGAGATCGACGAG |
| | | | AGCCTGATCTTCTACAAGAAGTGGGAGCTGGAGGCATGCGTGGACGCC |
| | | | GCCCTGCTGGCCACACAGATGGATAGGGTGAACGCCATCCCTTTTACCT |
| | | | ATGAGCAGCTGGATGTGCTGAAGCACAAGCTGCCACAGGGACAGGGAG |
| | | | GAGGAGGCAGCGGCGGAGGAGGCAATTCTCCAATGAGCGCCCGCATCC |
| | | | TGCAGGTGCAGGGACAGTCCGGACAGGGAGGACAGGTGCAGCTGGTGC |
| | | | AGTCTGGAGCCGAGGTGAAGAAGCCTGGGGCCAGCGTGAAGGTGTCCT |
| | | | GCAAGGCCTCCGGCTACTCTTTCACAGGCTATACCATGAACTGGGTGCG |
| | | | GCAGGCCCCAGGACAGGGACTGGAGTGGATGGGCCTGATCACACCCTA |
| | | | CAACGGGGCCAGCTCCTATAATCAGAAGTTTCGGGGCAAGGCCACCAT |
| | | | GACAGTGGACACCAGCACATCCACCGTGTACATGGAGCTGTCTAGCCTG |
| | | | AGATCCGAGGATACCGCCGTGTACTATTGTGCCAGAGGCGGCTACGAC |
| | | | GGCAGAGGCTTTGATTATTGGGGCCAGGGCACACTGGTGACCGTGTCCT |
| | | | CTGCTAGCACTAAGGGGCCTTCCGTGTTTCCACTGGCTCCCTCTAGTAA |
| | | | ATCCACCTCTGGAGGCACAGCTGCACTGGGATGTCTGGTGAAGGATTAC |
| | | | TTCCCTGAACCAGTCACAGTGAGTTGGAACTCAGGGGCTCTGACAAGTG |
| | | | GAGTCCATACTTTTCCCGCAGTGCTGCAGTCAAGCGGACTGTACTCCCT |
| | | | GTCCTCTGTGGTCACCGTGCCTAGTTCAAGCCTGGGCACCCAGACATAT |
| | | | ATCTGCAACGTGAATCACAAGCCATCAAATACAAAAGTCGACAAGAAA |
| | | | GTGGAGCCCAAGAGCTGTGATAAAACTCATACCTGCCCACCTTGTCCGG |
| | | | CGCCAGAGGCAGCAGGAGGACCAAGCGTGTTCCTGTTTCCACCCAAGC |
| | | | CCAAAGACACCCTGATGATTAGCCGAACCCCTGAAGTCACATGCGTGGT |
| | | | CGTGTCCGTGTCTCACGAGGACCCAGAAGTCAAGTTCAACTGGTACGTG |
| | | | GATGGCGTCGAGGTGCATAATGCCAAGACAAAACCCCGGGAGGAACAG |
| | | | TACAACAGCACCTATAGAGTCGTGTCCGTCCTGACAGTGCTGCACCAGG |
| | | | ATTGGCTGAACGGCAAGGAATATAAGTGCAAAGTGTCCAATAAGGCCC |
| | | | TGCCCGCTCCTATCGAGAAAACCATTTCTAAGGCAAAAGGCCAGCCTCG |
| | | | CGAACCACAGGTCTACGTGCTGCCTCCATCCCGGGACGAGCTGACAAA |
| | | | GAACCAGGTCTCTCTGCTGTGCCTGGTGAAAGGCTTCTATCCATCAGAT |
| | | | ATTGCTGTGGAGTGGGAAAGCAATGGGCAGCCCGAGAACAATTACCTG |
| | | | ACTTGGCCCCCTGTGCTGGACTCTGATGGGAGTTTCTTTCTGTATTCTAA |
| | | | GCTGACCGTGGATAAAAGTAGGTGGCAGCAGGGAAATGTCTTTAGTTG |
| | | | TTCAGTGATGCATGAAGCCCTGCATAACCACTACACCCAGAAAAGCCTG |
| | | | TCCCTGTCCCCCGGA |
| 100 | 17595 | Full nt | CAAGTGGGAGCCTGCCCTAGCGGCAAGAAGGCCCGCGAGATCGACGAG |
| | | | TCCCTGATCTTCTACAAGAAGTGGGAGCTGGAGGCATGCGTGGACGCC |
| | | | GCCCTGCTGGCCACACAGATGGATAGGGTGAACGCCATCCCTTTTACCT |
| | | | ATGAGCAGCTGGATGTGCTGAAGCACAAGCTGCCACAGGGACAGGGAG |
| | | | GAGGAGGCTCCGGAGGAGGCGGCAACTCTCCAGGGAAGGGGCGGAGC |
| | | | GCAAATGCCCAGGGACAGTCTGGACAGGGAGGACAGGTGCAGCTGGTG |
| | | | CAGAGCGGAGCCGAGGTGAAGAAGCCTGGGGCCAGCGTGAAGGTGTCT |
| | | | TGCAAGGCCTCTGGCTACAGCTTCACAGGCTATACCATGAATTGGGTGC |
| | | | GGCAGGCCCCAGGACAGGGACTGGAGTGGATGGGCCTGATCACACCCT |
| | | | ACAACGGGGCCAGCTCCTATAATCAGAAGTTTCGGGGCAAGGCCACCA |
| | | | TGACAGTGGACACCAGCACATCCACCGTGTACATGGAGCTGTCTAGCCT |
| | | | GAGAAGCGAGGATACCGCCGTGTACTATTGTGCCAGAGGCGGCTACGA |
| | | | CGGCAGAGGCTTTGATTATTGGGGCCAGGGCACACTGGTGACCGTGTCC |
| | | | TCTGCTAGCACTAAGGGGCCTTCCGTGTTTCCACTGGCTCCCTCTAGTAA |
| | | | ATCCACCTCTGGAGGCACAGCTGCACTGGGATGTCTGGTGAAGGATTAC |
| | | | TTCCCTGAACCAGTCACAGTGAGTTGGAACTCAGGGGCTCTGACAAGTG |
| | | | GAGTCCATACTTTTCCCGCAGTGCTGCAGTCAAGCGGACTGTACTCCCT |
| | | | GTCCTCTGTGGTCACCGTGCCTAGTTCAAGCCTGGGCACCCAGACATAT |
| | | | ATCTGCAACGTGAATCACAAGCCATCAAATACAAAAGTCGACAAGAAA |
| | | | GTGGAGCCCAAGAGCTGTGATAAAACTCATACCTGCCCACCTTGTCCGG |
| | | | CGCCAGAGGCAGCAGGAGGACCAAGCGTGTTCCTGTTTCCACCCAAGC |
| | | | CCAAAGACACCCTGATGATTAGCCGAACCCCTGAAGTCACATGCGTGGT |
| | | | CGTGTCCGTGTCTCACGAGGACCCAGAAGTCAAGTTCAACTGGTACGTG |
| | | | GATGGCGTCGAGGTGCATAATGCCAAGACAAAACCCCGGGAGGAACAG |
| | | | TACAACAGCACCTATAGAGTCGTGTCCGTCCTGACAGTGCTGCACCAGG |
| | | | ATTGGCTGAACGGCAAGGAATATAAGTGCAAAGTGTCCAATAAGGCCC |
| | | | TGCCCGCTCCTATCGAGAAAACCATTTCTAAGGCAAAAGGCCAGCCTCG |
| | | | CGAACCACAGGTCTACGTGCTGCCTCCATCCCGGGACGAGCTGACAAA |
| | | | GAACCAGGTCTCTCTGCTGTGCCTGGTGAAAGGCTTCTATCCATCAGAT |
| | | | ATTGCTGTGGAGTGGGAAAGCAATGGGCAGCCCGAGAACAATTACCTG |
| | | | ACTTGGCCCCCTGTGCTGGACTCTGATGGGAGTTTCTTTCTGTATTCTAA |
| | | | GCTGACCGTGGATAAAAGTAGGTGGCAGCAGGGAAATGTCTTTAGTTG |
| | | | TTCAGTGATGCATGAAGCCCTGCATAACCACTACACCCAGAAAAGCCTG |
| | | | TCCCTGTCCCCCGGA |
| 101 | 17601 | Full nt | CAAGTGGGAGCCTGCCCTAGCGGCAAGAAGGCCCGCGAGATCGACGAG |
| | | | TCCCTGATCTTCTACAAGAAGTGGGAGCTGGAGGCATGCGTGGACGCC |
| | | | GCCCTGCTGGCCACACAGATGGATAGGGTGAACGCCATCCCTTTTACCT |
| | | | ATGAGCAGCTGGATGTGCTGAAGCACAAGCTGCCACAGGGACAGGGAG |
| | | | GAGGAGGCTCCGGAGGAGGCGGCAACTCTCCAATGAGCGGGCGGAGCG |
| | | | CAAATGCCCAGGGACAGTCTGGACAGGGAGGACAGGTGCAGCTGGTGC |
| | | | AGAGCGGAGCCGAGGTGAAGAAGCCTGGGGCCAGCGTGAAGGTGTCTT |
| | | | GCAAGGCCTCTGGCTACAGCTTCACAGGCTATACCATGAATTGGGTGCG |
| | | | GCAGGCCCCAGGACAGGGACTGGAGTGGATGGGCCTGATCACACCCTA |
| | | | CAACGGGGCCAGCTCCTATAATCAGAAGTTTCGGGGCAAGGCCACCAT |
| | | | GACAGTGGACACCAGCACATCCACCGTGTACATGGAGCTGTCTAGCCTG |
| | | | AGAAGCGAGGATACCGCCGTGTACTATTGTGCCAGAGGCGGCTACGAC |
| | | | GGCAGAGGCTTTGATTATTGGGGCCAGGGCACACTGGTGACCGTGTCCT |
| | | | CTGCTAGCACTAAGGGGCCTTCCGTGTTTCCACTGGCTCCCTCTAGTAA |
| | | | ATCCACCTCTGGAGGCACAGCTGCACTGGGATGTCTGGTGAAGGATTAC |
| | | | TTCCCTGAACCAGTCACAGTGAGTTGGAACTCAGGGGCTCTGACAAGTG |
| | | | GAGTCCATACTTTTCCCGCAGTGCTGCAGTCAAGCGGACTGTACTCCCT |
| | | | GTCCTCTGTGGTCACCGTGCCTAGTTCAAGCCTGGGCACCCAGACATAT |
| | | | ATCTGCAACGTGAATCACAAGCCATCAAATACAAAAGTCGACAAGAAA |
| | | | GTGGAGCCCAAGAGCTGTGATAAAACTCATACCTGCCCACCTTGTCCGG |
| | | | CGCCAGAGGCAGCAGGAGGACCAAGCGTGTTCCTGTTTCCACCCAAGC |
| | | | CCAAAGACACCCTGATGATTAGCCGAACCCCTGAAGTCACATGCGTGGT |
| | | | CGTGTCCGTGTCTCACGAGGACCCAGAAGTCAAGTTCAACTGGTACGTG |
| | | | GATGGCGTCGAGGTGCATAATGCCAAGACAAAACCCCGGGAGGAACAG |
| | | | TACAACAGCACCTATAGAGTCGTGTCCGTCCTGACAGTGCTGCACCAGG |
| | | | ATTGGCTGAACGGCAAGGAATATAAGTGCAAAGTGTCCAATAAGGCCC |
| | | | TGCCCGCTCCTATCGAGAAAACCATTTCTAAGGCAAAAGGCCAGCCTCG |
| | | | CGAACCACAGGTCTACGTGCTGCCTCCATCCCGGGACGAGCTGACAAA |
| | | | GAACCAGGTCTCTCTGCTGTGCCTGGTGAAAGGCTTCTATCCATCAGAT |
| | | | ATTGCTGTGGAGTGGGAAAGCAATGGGCAGCCCGAGAACAATTACCTG |
| | | | ACTTGGCCCCCTGTGCTGGACTCTGATGGGAGTTTCTTTCTGTATTCTAA |
| | | | GCTGACCGTGGATAAAAGTAGGTGGCAGCAGGGAAATGTCTTTAGTTG |
| | | | TTCAGTGATGCATGAAGCCCTGCATAACCACTACACCCAGAAAAGCCTG |
| | | | TCCCTGTCCCCCGGA |
| 102 | 17871 | Full nt | ATCTGGGAGCTGAAGAAGGACGTGTACGTGGTGGAGCTGGACTGGTAT |
| | | | CCAGATGCACCAGGAGAGATGGTGGTGCTGACCTGCGACACACCAGAG |
| | | | GAGGATGGCATCACCTGGACACTGGACCAGAGCTCCGAGGTGCTGGGC |
| | | | AGCGGCAAGACCCTGACAATCCAGGTGAAGGAGTTCGGCGATGCCGGA |
| | | | CAGTACACATGTCACAAGGGCGGCGAGGTGCTGTCTCACAGCCTGCTGC |
| | | | TGCTGCACAAGAAGGAGGATGGCATCTGGTCCACAGACATCCTGAAGG |
| | | | ATCAGAAGGAGCCCAAGAACAAGACCTTCCTGAGGTGCGAGGCCAAGA |
| | | | ATTATTCTGGCCGCTTTACCTGTTGGTGGCTGACCACAATCTCTACCGAC |
| | | | CTGACCTTCAGCGTGAAGTCTAGCCGGGGCTCCTCTGATCCTCAGGGAG |
| | | | TGACATGCGGAGCCGCCACCCTGAGCGCCGAGCGGGTGAGAGGCGACA |
| | | | ACAAGGAGTACGAGTATAGCGTGGAGTGCCAGGAGGATTCCGCCTGTC |
| | | | CCGCCGCCGAGGAGTCCCTGCCTATCGAAGTGATGGTGGACGCCGTGC |
| | | | ACAAGCTGAAGTACGAGAATTATACAAGCTCCTTCTTTATCAGGGACAT |
| | | | CATCAAGCCAGATCCCCCTAAGAACCTGCAGCTGAAGCCCCTGAAGAA |
| | | | CAGCAGACAGGTGGAGGTGTCTTGGGAGTACCCTGATACCTGGTCCAC |
| | | | ACCACACAGCTATTTCTCCCTGACCTTTTGCGTGCAGGTGCAGGGCAAG |
| | | | TCTAAGCGGGAGAAGAAGGACAGAGTGTTTACCGATAAGACAAGCGCC |
| | | | ACCGTGATCTGTAGAAAGAACGCCTCCATCTCTGTGAGGGCCCAGGACC |
| | | | GCTACTATTCTAGCTCCTGGTCTGAGTGGGCCAGCGTGCCTTGTTCC |
| 103 | 17872 | Full nt | AGAAACCTGCCCGTGGCCACACCCGATCCTGGCATGTTTCCCTGCCTGC |
| | | | ACCACAGCCAGAACCTGCTGCGGGCCGTGTCCAATATGCTGCAGAAGG |
| | | | CCAGACAGACCCTGGAGTTCTACCCCTGTACATCTGAGGAGATCGACCA |
| | | | CGAGGATATCACCAAGGACAAGACCTCCACAGTGGAGGCCTGCCTGCC |
| | | | TCTGGAGCTGACAAAGAACGAGTCCTGTCTGAACAGCCGGGAGACCAG |
| | | | CTTCATCACAAATGGCTCCTGCCTGGCCTCTCGCAAGACCAGCTTTATG |
| | | | ATGGCCCTGTGCCTGAGCTCCATCTACGAGGATCTGAAGATGTATCAGG |
| | | | TGGAGTTCAAGACAATGAACGCCAAGCTGCTGATGGACCCTAAGCGGC |
| | | | AGATCTTTCTGGATCAGAATATGCTGGCCGTGATCGACGAGCTGATGCA |
| | | | GGCCCTGAACTTTAATAGCGAGACCGTGCCTCAGAAGTCTAGCCTGGAG |
| | | | GAGCCAGATTTCTACAAGACAAAGATCAAGCTGTGCATCCTGCTGCACG |
| | | | CCTTCCGGATCAGAGCCGTGACCATCGACAGAGTGATGTCCTATCTGAA |
| | | | CGCCTCT |
| 104 | 17875 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCCCCTTCCAGAGATGAGCTGACCAAGAA |
| | | | CCAGGTGTCTCTGACATGCCTGGTGAAGGGCTTCTACCCTAGCGACATC |
| | | | GCCGTGGAGTGGGAGTCCAATGGCCAGCCAGAGAACAATTATAAGACC |
| | | | ACACCACCCGTGCTGGACAGCGATGGCTCCTTCGCCCTGGTGTCCAAGC |
| | | | TGACCGTGGATAAGTCTAGGTGGCAGCAGGGCAACGTGTTTTCCTGTTC |
| | | | TGTGATGCACGAGGCCCTGCACAATCACTACACCCAGAAGAGCCTGTCC |
| | | | CTGTCTCCCGGCAGAAACCTGCCTGTGGCCACACCTGACCCAGGCATGT |
| | | | TCCCATGCCTGCACCACTCTCAGAACCTGCTGAGGGCCGTGAGCAATAT |
| | | | GCTGCAGAAGGCCCGCCAGACCCTGGAGTTTTATCCCTGTACATCCGAG |
| | | | GAGATCGACCACGAGGATATCACCAAGGATAAGACCAGCACAGTGGAG |
| | | | GCCTGCCTGCCTCTGGAGCTGACAAAGAACGAGTCTTGTCTGAATAGCC |
| | | | GGGAGACCTCCTTCATCACAAATGGCTCTTGCCTGGCCAGCAGAAAGAC |
| | | | CTCCTTTATGATGGCCCTGTGCCTGAGCTCCATCTACGAGGACCTGAAG |
| | | | ATGTATCAGGTGGAGTTCAAGACAATGAACGCCAAGCTGCTGATGGAC |
| | | | CCCAAGCGGCAGATCTTTCTGGATCAGAATATGCTGGCCGTGATCGACG |
| | | | AGCTGATGCAGGCCCTGAACTTTAATTCCGAGACCGTGCCACAGAAGTC |
| | | | TAGCCTGGAGGAGCCCGATTTCTACAAGACAAAGATCAAGCTGTGCAT |
| | | | CCTGCTGCACGCCTTTCGGATCAGAGCCGTGACCATCGACAGAGTGATG |
| | | | TCTTATCTGAACGCCAGC |
| 105 | 17876 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAAGTCTAAGAGGGAGAAG |
| | | | AAGGACCGCGTGTTCACCGATAAGACAAGCGCCACCGTGATCTGTCGG |
| | | | AAGAACGCCAGCATCTCCGTGCGGGCCCAGGATAGATACTATTCTAGCT |
| | | | CCTGGAGCGAGTGGGCCTCCGTGCCATGTTCTGGAGGAGGAGGCAGCG |
| | | | GCGGAGGAGGCTCCGGCGGCGGCGGCTCTAGAAATCTGCCAGTGGCCA |
| | | | CCCCTGACCCAGGCATGTTCCCCTGCCTGCACCACTCTCAGAACCTGCT |
| | | | GCGGGCCGTGAGCAATATGCTGCAGAAGGCCAGACAGACACTGGAGTT |
| | | | TTACCCATGTACCTCCGAGGAGATCGACCACGAGGATATCACAAAGGA |
| | | | TAAGACCTCTACAGTGGAGGCATGCCTGCCACTGGAGCTGACCAAGAA |
| | | | CGAGAGCTGTCTGAACAGCCGGGAGACATCTTTCATCACCAACGGCAG |
| | | | CTGCCTGGCCTCCAGAAAGACATCTTTTATGATGGCCCTGTGCCTGTCT |
| | | | AGCATCTACGAGGACCTGAAGATGTATCAGGTGGAGTTCAAGACCATG |
| | | | AACGCCAAGCTGCTGATGGACCCTAAGAGGCAGATCTTTCTGGATCAG |
| | | | AATATGCTGGCCGTGATCGACGAGCTGATGCAGGCCCTGAACTTCAATA |
| | | | GCGAGACAGTGCCACAGAAGTCCTCTCTGGAGGAGCCCGATTTCTACA |
| | | | AGACCAAGATCAAGCTGTGCATCCTGCTGCACGCCTTCCGGATCAGAGC |
| | | | CGTGACCATCGACCGCGTGATGAGCTACCTGAACGCCAGC |
| 106 | 17877 | Full nt | AGAAACCTGCCAGTGGCCACACCAGATCCCGGCATGTTTCCATGCCTGC |
| | | | ACCACTCCCAGAACCTGCTGCGGGCCGTGTCTAATATGCTGCAGAAGGC |
| | | | CAGACAGACCCTGGAGTTCTACCCATGTACAAGCGAGGAGATCGACCA |
| | | | CGAGGATATCACCAAGGACAAGACCTCCACAGTGGAGGCATGCCTGCC |
| | | | ACTGGAGCTGACAAAGAACGAGAGCTGTCTGAACAGCCGGGAGACCAG |
| | | | CTTCATCACAAATGGCAGCTGCCTGGCCTCCCGCAAGACCTCTTTTATG |
| | | | ATGGCCCTGTGCCTGAGCTCCATCTACGAGGATCTGAAGATGTATCAGG |
| | | | TGGAGTTCAAGACAATGAACGCCAAGCTGCTGATGGACCCCAAGCGGC |
| | | | AGATCTTTCTGGATCAGAATATGCTGGCCGTGATCGACGAGCTGATGCA |
| | | | GGCCCTGAACTTTAATAGCGAGACCGTGCCCCAGAAGTCTAGCCTGGA |
| | | | GGAGCCTGATTTCTACAAGACAAAGATCAAGCTGTGCATCCTGCTGCAC |
| | | | GCCTTCCGGATCAGAGCCGTGACCATCGACAGAGTGATGAGCTATCTGA |
| | | | ACGCCTCCGGAGGAGGAGGCTCTGGAGGAGGCGGCAGCGAGCCTAAGT |
| | | | CCTCTGACAAGACCCACACATGCCCCCCTTGTCCGGCGCCAGAGGCTGC |
| | | | AGGAGGACCAAGCGTGTTCCTGTTTCCACCCAAGCCTAAAGACACACTG |
| | | | ATGATTTCCCGAACCCCCGAAGTCACATGCGTGGTCGTGTCTGTGAGTC |
| | | | ACGAGGACCCTGAAGTCAAGTTCAACTGGTACGTGGATGGCGTCGAGG |
| | | | TGCATAATGCCAAGACTAAACCTAGGGAGGAACAGTACAACTCAACCT |
| | | | ATCGCGTCGTGAGCGTCCTGACAGTGCTGCACCAGGATTGGCTGAACGG |
| | | | CAAAGAATATAAGTGCAAAGTGAGCAATAAGGCCCTGCCCGCTCCTAT |
| | | | CGAGAAAACCATTTCCAAGGCTAAAGGGCAGCCTCGCGAACCACAGGT |
| | | | CTACGTGTATCCTCCAAGCCGGGACGAGCTGACAAAGAACCAGGTCTC |
| | | | CCTGACTTGTCTGGTGAAAGGGTTTTACCCTAGTGATATCGCTGTGGAG |
| | | | TGGGAATCAAATGGACAGCCAGAGAACAATTATAAGACTACCCCCCCT |
| | | | GTGCTGGACAGTGATGGGTCATTCGCACTGGTCTCCAAGCTGACAGTGG |
| | | | ACAAATCTCGGTGGCAGCAGGGAAATGTCTTTTCATGTAGCGTGATGCA |
| | | | TGAAGCACTGCACAACCATTACACCCAGAAGTCACTGTCACTGTCACCA |
| | | | GGA |
| 107 | 17879 | Full nt | ATCTGGGAGCTGAAGAAGGACGTGTACGTGGTGGAGCTGGACTGGTAT |
| | | | CCTGATGCCCCAGGCGAGATGGTGGTGCTGACCTGCGACACACCCGAG |
| | | | GAGGATGGCATCACCTGGACACTGGACCAGAGCTCCGAGGTGCTGGGC |
| | | | AGCGGCAAGACCCTGACAATCCAGGTGAAGGAGTTCGGCGATGCCGGA |
| | | | CAGTACACATGTCACAAGGGCGGCGAGGTGCTGTCCCACTCTCTGCTGC |
| | | | TGCTGCACAAGAAGGAGGATGGCATCTGGTCTACAGACATCCTGAAGG |
| | | | ATCAGAAGGAGCCTAAGAACAAGACCTTCCTGAGGTGCGAGGCCAAGA |
| | | | ATTATAGCGGCAGATTCACCTGTTGGTGGCTGACCACAATCAGCACCGA |
| | | | CCTGACATTTTCCGTGAAGTCTAGCCGGGGCTCCTCTGATCCACAGGGA |
| | | | GTGACATGCGGAGCCGCCACCCTGAGCGCCGAGCGGGTGAGAGGCGAC |
| | | | AACAAGGAGTACGAGTATTCCGTGGAGTGCCAGGAGGATTCTGCCTGT |
| | | | CCAGCCGCCGAGGAGTCCCTGCCAATCGAAGTGATGGTGGACGCCGTG |
| | | | CACAAGCTGAAGTACGAGAATTATACAAGCTCCTTCTTTATCAGGGACA |
| | | | TCATCAAGCCTGATCCCCCTAAGAACCTGCAGCTGAAGCCCCTGAAGAA |
| | | | CAGCAGACAGGTGGAGGTGAGCTGGGAGTACCCCGATACCTGGAGCAC |
| | | | ACCTCACTCTTATTTCAGCCTGACCTTTTGCGTGCAGGTGCAGGGCAAG |
| | | | AGCAAGCGGGAGAAGAAGGACAGAGTGTTTACCGATAAGACATCCGCC |
| | | | ACCGTGATCTGTAGAAAGAACGCCAGCATCTCCGTGAGGGCACAGGAC |
| | | | CGCTACTATTCTAGCTCCTGGTCCGAGTGGGCCTCTGTGCCCTGTAGCG |
| | | | GAGGAGGAGGCTCCGGAGGAGGAGGCTCTGAGCCTAAGTCTAGCGATA |
| | | | AGACCCACACATGCCCACCCTGTCCGGCGCCAGAGGCTGCAGGAGGAC |
| | | | CAAGCGTGTTCCTGTTTCCACCCAAGCCTAAAGACACACTGATGATTTC |
| | | | CCGAACCCCCGAAGTCACATGCGTGGTCGTGTCTGTGAGTCACGAGGAC |
| | | | CCTGAAGTCAAGTTCAACTGGTACGTGGATGGCGTCGAGGTGCATAATG |
| | | | CCAAGACTAAACCTAGGGAGGAACAGTACAACTCAACCTATCGCGTCG |
| | | | TGAGCGTCCTGACAGTGCTGCACCAGGATTGGCTGAACGGCAAAGAAT |
| | | | ATAAGTGCAAAGTGAGCAATAAGGCCCTGCCCGCTCCTATCGAGAAAA |
| | | | CCATTTCCAAGGCTAAAGGGCAGCCTCGCGAACCACAGGTCTACGTGTA |
| | | | TCCTCCAAGCCGGGACGAGCTGACAAAGAACCAGGTCTCCCTGACTTGT |
| | | | CTGGTGAAAGGGTTTTACCCTAGTGATATCGCTGTGGAGTGGGAATCAA |
| | | | ATGGACAGCCAGAGAACAATTATAAGACTACCCCCCCTGTGCTGGACA |
| | | | GTGATGGGTCATTCGCACTGGTCTCCAAGCTGACAGTGGACAAATCTCG |
| | | | GTGGCAGCAGGGAAATGTCTTTTCATGTAGCGTGATGCATGAAGCACTG |
| | | | CACAACCATTACACCCAGAAGTCACTGTCACTGTCACCAGGA |
| 108 | 17880 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ACCAGGTGTCTCTGCTGTGCCTGGTGAAGGGCTTTTATCCTAGCGATAT |
| | | | CGCCGTGGAGTGGGAGTCCAATGGCCAGCCAGAGAACAATTACCTGAC |
| | | | CTGGCCACCCGTGCTGGACTCTGATGGCAGCTTCTTTCTGTATTCCAAGC |
| | | | TGACAGTGGACAAGTCTCGGTGGCAGCAGGGCAACGTGTTCTCTTGTAG |
| | | | CGTGATGCACGAGGCCCTGCACAATCACTACACCCAGAAGTCCCTGAG |
| | | | CTTAAGCCCTGGAGGAGGAGGAGGCTCTGGAGGAGGAGGCAGCATCTG |
| | | | GGAGCTGAAGAAGGACGTGTACGTGGTGGAGCTGGACTGGTATCCAGA |
| | | | TGCACCAGGAGAGATGGTGGTGCTGACCTGCGACACACCAGAGGAGGA |
| | | | TGGCATCACCTGGACACTGGACCAGAGCTCCGAGGTGCTGGGCAGCGG |
| | | | CAAGACCCTGACAATCCAGGTGAAGGAGTTCGGCGATGCCGGACAGTA |
| | | | CACATGTCACAAGGGCGGCGAGGTGCTGTCTCACAGCCTGCTGCTGCTG |
| | | | CACAAGAAGGAGGATGGCATCTGGAGCACAGACATCCTGAAGGATCAG |
| | | | AAGGAGCCCAAGAACAAGACCTTCCTGAGGTGCGAGGCCAAGAATTAT |
| | | | AGCGGCAGATTCACCTGTTGGTGGCTGACCACAATCTCCACCGACCTGA |
| | | | CATTTTCTGTGAAGTCTAGCAGGGGCTCCTCTGATCCTCAGGGAGTGAC |
| | | | ATGCGGAGCCGCCACCCTGTCTGCCGAGCGGGTGAGAGGCGACAACAA |
| | | | GGAGTACGAGTATTCTGTGGAGTGCCAGGAGGATAGCGCCTGTCCCGC |
| | | | CGCCGAGGAGTCCCTGCCTATCGAAGTGATGGTGGACGCCGTGCACAA |
| | | | GCTGAAGTACGAGAATTATACAAGCTCCTTCTTTATCCGGGACATCATC |
| | | | AAGCCAGATCCCCCTAAGAACCTGCAGCTGAAGCCCCTGAAGAATAGC |
| | | | AGACAGGTGGAGGTGTCCTGGGAGTACCCTGATACCTGGTCCACACCA |
| | | | CACAGCTATTTCTCCCTGACCTTTTGCGTGCAGGTGCAGGGCAAGTCCA |
| | | | AGCGGGAGAAGAAGGACAGAGTGTTCACCGATAAGACATCTGCCACCG |
| | | | TGATCTGTCGGAAGAACGCCTCCATCTCTGTGAGGGCCCAGGACCGCTA |
| | | | CTATTCTAGCTCCTGGTCTGAGTGGGCCAGCGTGCCCTGTTCC |
| 109 | 17881 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ACCAGGTGTCTCTGCTGTGCCTGGTGAAGGGCTTTTATCCTAGCGATAT |
| | | | CGCCGTGGAGTGGGAGTCCAATGGCCAGCCAGAGAACAATTACCTGAC |
| | | | CTGGCCACCCGTGCTGGACTCTGATGGCAGCTTCTTTCTGTATTCCAAGC |
| | | | TGACAGTGGACAAGTCTCGGTGGCAGCAGGGCAACGTGTTCTCTTGTAG |
| | | | CGTGATGCACGAGGCCCTGCACAATCACTACACCCAGAAGTCCCTGAG |
| | | | CTTAAGCCCAGGCCGGAACCTGCCAGTGGCCACCCCCGATCCTGGCATG |
| | | | TTCCCATGCCTGCACCACAGCCAGAACCTGCTGAGGGCCGTGTCCAATA |
| | | | TGCTGCAGAAGGCCCGCCAGACCCTGGAGTTTTACCCCTGTACATCTGA |
| | | | GGAGATCGACCACGAGGATATCACCAAGGACAAGACCTCCACAGTGGA |
| | | | GGCCTGCCTGCCTCTGGAGCTGACAAAGAACGAGTCCTGTCTGAACAGC |
| | | | CGGGAGACCAGCTTCATCACAAATGGCTCCTGCCTGGCCTCTAGAAAGA |
| | | | CCAGCTTTATGATGGCCCTGTGCCTGAGCTCCATCTACGAGGATCTGAA |
| | | | GATGTATCAGGTGGAGTTCAAGACAATGAACGCCAAGCTGCTGATGGA |
| | | | CCCCAAGAGGCAGATCTTTCTGGATCAGAATATGCTGGCCGTGATCGAC |
| | | | GAGCTGATGCAGGCCCTGAACTTCAATAGCGAGACCGTGCCTCAGAAG |
| | | | TCTAGCCTGGAGGAGCCAGATTTCTACAAGACAAAGATCAAGCTGTGC |
| | | | ATCCTGCTGCACGCCTTTCGGATCAGAGCCGTGACAATCGACCGCGTGA |
| | | | TGTCCTATCTGAACGCCTCT |
| 110 | 17906 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCCCAGGTGTACGTGTATCCCCCTTCTCGGGACGAGCTGACCAAGAA |
| | | | CCAGGTGAGCCTGACATGCCTGGTGAAGGGCTTCTACCCATCCGATATC |
| | | | GCCGTGGAGTGGGAGTCTAATGGCCAGCCCGAGAACAATTATAAGACC |
| | | | ACACCACCCGTGCTGGACTCCGATGGCTCTTTCGCCCTGGTGTCTAAGC |
| | | | TGACCGTGGACAAGAGCAGGTGGCAGCAGGGCAACGTGTTTTCCTGCT |
| | | | CTGTGATGCACGAGGCCCTGCACAATCACTACACCCAGAAGAGCCTGA |
| | | | GCTTAAGCCCTGGCAGGGCAGTGCCAGGAGGCAGCTCCCCCGCCTGGA |
| | | | CCCAGTGCCAGCAGCTGAGCCAGAAGCTGTGCACACTGGCCTGGTCCG |
| | | | CCCACCCACTGGTGGGACACATGGACCTGAGAGAGGAGGGCGATGAGG |
| | | | AGACCACAAACGACGTGCCTCACATCCAGTGCGGCGACGGCTGTGATC |
| | | | CACAGGGCCTGAGGGATAATTCTCAGTTCTGCCTGCAGCGCATCCACCA |
| | | | GGGCCTGATCTTCTACGAGAAGCTGCTGGGCAGCGATATCTTTACCGGA |
| | | | GAGCCATCTCTGCTGCCAGACAGCCCTGTGGGACAGCTGCACGCCTCCC |
| | | | TGCTGGGCCTGTCTCAGCTGCTGCAGCCAGAGGGACACCACTGGGAGA |
| | | | CACAGCAGATCCCTTCTCTGAGCCCATCCCAGCCATGGCAGCGGCTGCT |
| | | | GCTGCGGTTCAAGATCCTGCGGTCCCTGCAGGCCTTCGTGGCAGTGGCA |
| | | | GCAAGAGTGTTTGCACACGGAGCCGCCACCCTGTCTCCT |
| 111 | 17907 | Full nt | AGAGCAGTGCCAGGCGGCAGCTCCCCTGCCTGGACCCAGTGCCAGCAG |
| | | | CTGTCCCAGAAGCTGTGCACACTGGCCTGGTCTGCCCACCCTCTGGTGG |
| | | | GACACATGGACCTGCGGGAGGAGGGCGATGAGGAGACCACAAACGAC |
| | | | GTGCCACACATCCAGTGCGGCGACGGATGTGATCCACAGGGCCTGCGG |
| | | | GATAATAGCCAGTTCTGCCTGCAGAGAATCCACCAGGGCCTGATCTTCT |
| | | | ACGAGAAGCTGCTGGGCTCCGATATCTTTACCGGAGAGCCATCCCTGCT |
| | | | GCCAGACTCTCCAGTGGGACAGCTGCACGCCAGCCTGCTGGGCCTGTCC |
| | | | CAGCTGCTGCAGCCTGAGGGCCACCACTGGGAGACACAGCAGATCCCA |
| | | | TCCCTGTCTCCTAGCCAGCCATGGCAGAGGCTGCTGCTGCGCTTTAAGA |
| | | | TCCTGAGGTCTCTGCAGGCCTTCGTGGCAGTGGCAGCACGCGTGTTTGC |
| | | | CCACGGAGCCGCCACACTGAGCCCAGGAGGAGGAGGCTCTGGAGGAGG |
| | | | AGGCAGCGAGCCTAAGTCTAGCGACAAGACCCACACATGCCCCCCTTG |
| | | | TCCGGCGCCAGAGGCTGCAGGAGGACCAAGCGTGTTCCTGTTTCCACCC |
| | | | AAGCCTAAAGACACACTGATGATTTCCCGAACCCCCGAAGTCACATGC |
| | | | GTGGTCGTGTCTGTGAGTCACGAGGACCCTGAAGTCAAGTTCAACTGGT |
| | | | ACGTGGATGGCGTCGAGGTGCATAATGCCAAGACTAAACCTAGGGAGG |
| | | | AACAGTACAACTCAACCTATCGCGTCGTGAGCGTCCTGACAGTGCTGCA |
| | | | CCAGGATTGGCTGAACGGCAAAGAATATAAGTGCAAAGTGAGCAATAA |
| | | | GGCCCTGCCCGCTCCTATCGAGAAAACCATTTCCAAGGCTAAAGGGCA |
| | | | GCCTCGCGAACCACAGGTCTACGTGTATCCTCCAAGCCGGGACGAGCTG |
| | | | ACAAAGAACCAGGTCTCCCTGACTTGTCTGGTGAAAGGGTTTTACCCTA |
| | | | GTGATATCGCTGTGGAGTGGGAATCAAATGGACAGCCAGAGAACAATT |
| | | | ATAAGACTACCCCCCCTGTGCTGGACAGTGATGGGTCATTCGCACTGGT |
| | | | CTCCAAGCTGACAGTGGACAAATCTCGGTGGCAGCAGGGAAATGTCTTT |
| | | | TCATGTAGCGTGATGCATGAAGCACTGCACAACCATTACACCCAGAAGT |
| | | | CACTGTCACTGTCACCAGGA |
| 112 | 17908 | Full nt | AGAGCCGTGCCTGGCGGCAGCTCCCCAGCCTGGACCCAGTGCCAGCAG |
| | | | CTGAGCCAGAAGCTGTGCACACTGGCCTGGTCCGCCCACCCACTGGTGG |
| | | | GACACATGGACCTGCGGGAGGAGGGCGATGAGGAGACCACAAACGAC |
| | | | GTGCCACACATCCAGTGCGGCGACGGATGTGATCCTCAGGGCCTGCGG |
| | | | GATAATTCTCAGTTCTGCCTGCAGAGAATCCACCAGGGCCTGATCTTCT |
| | | | ACGAGAAGCTGCTGGGCAGCGATATCTTTACCGGAGAGCCTTCTCTGCT |
| | | | GCCAGACAGCCCTGTGGGACAGCTGCACGCCTCCCTGCTGGGCCTGTCT |
| | | | CAGCTGCTGCAGCCAGAGGGCCACCACTGGGAGACACAGCAGATCCCC |
| | | | TCTCTGAGCCCATCCCAGCCATGGCAGAGGCTGCTGCTGCGCTTTAAGA |
| | | | TCCTGAGGTCCCTGCAGGCCTTCGTGGCAGTGGCAGCACGCGTGTTTGC |
| | | | CCACGGAGCCGCCACACTGTCTCCC |
| 113 | 17942 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCCCAGGTGTACGTGTATCCCCCTTCTCGGGACGAGCTGACCAAGAA |
| | | | CCAGGTGAGCCTGACATGCCTGGTGAAGGGCTTCTACCCATCCGATATC |
| | | | GCCGTGGAGTGGGAGTCTAATGGCCAGCCCGAGAACAATTATAAGACC |
| | | | ACACCACCCGTGCTGGACTCCGATGGCTCTTTCGCCCTGGTGTCTAAGC |
| | | | TGACCGTGGACAAGAGCAGGTGGCAGCAGGGCAACGTGTTTTCCTGCT |
| | | | CTGTGATGCACGAGGCCCTGCACAATCACTACACCCAGAAGAGCCTGA |
| | | | GCTTAAGCCCTGGAGGAGGAGGAGGCTCTGGAGGAGGAGGCAGCATCT |
| | | | GGGAGCTGAAGAAGGACGTGTACGTGGTGGAGCTGGACTGGTATCCAG |
| | | | ATGCACCAGGAGAGATGGTGGTGCTGACCTGCGACACACCAGAGGAGG |
| | | | ATGGCATCACCTGGACACTGGACCAGAGCTCCGAGGTGCTGGGCAGCG |
| | | | GCAAGACCCTGACAATCCAGGTGAAGGAGTTCGGCGATGCCGGACAGT |
| | | | ACACATGTCACAAGGGCGGCGAGGTGCTGTCTCACAGCCTGCTGCTGCT |
| | | | GCACAAGAAGGAGGATGGCATCTGGAGCACAGACATCCTGAAGGATCA |
| | | | GAAGGAGCCCAAGAACAAGACCTTCCTGAGGTGCGAGGCCAAGAATTA |
| | | | TAGCGGCAGATTCACCTGTTGGTGGCTGACCACAATCTCCACCGACCTG |
| | | | ACATTTTCTGTGAAGTCTAGCAGGGGCTCCTCTGATCCTCAGGGAGTGA |
| | | | CATGCGGAGCCGCCACCCTGTCTGCCGAGCGGGTGAGAGGCGACAACA |
| | | | AGGAGTACGAGTATTCTGTGGAGTGCCAGGAGGATAGCGCCTGTCCCG |
| | | | CCGCCGAGGAGTCCCTGCCTATCGAAGTGATGGTGGACGCCGTGCACA |
| | | | AGCTGAAGTACGAGAATTATACAAGCTCCTTCTTTATCCGGGACATCAT |
| | | | CAAGCCAGATCCCCCTAAGAACCTGCAGCTGAAGCCCCTGAAGAATAG |
| | | | CAGACAGGTGGAGGTGTCCTGGGAGTACCCTGATACCTGGTCCACACC |
| | | | ACACAGCTATTTCTCCCTGACCTTTTGCGTGCAGGTGCAGGGCAAGTCC |
| | | | AAGCGGGAGAAGAAGGACAGAGTGTTCACCGATAAGACATCTGCCACC |
| | | | GTGATCTGTCGGAAGAACGCCTCCATCTCTGTGAGGGCCCAGGACCGCT |
| | | | ACTATTCTAGCTCCTGGTCTGAGTGGGCCAGCGTGCCCTGTTCC |
| 114 | 17945 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCCCAGGTGTACGTGTATCCCCCTTCTCGGGACGAGCTGACCAAGAA |
| | | | CCAGGTGAGCCTGACATGCCTGGTGAAGGGCTTCTACCCATCCGATATC |
| | | | GCCGTGGAGTGGGAGTCTAATGGCCAGCCCGAGAACAATTATAAGACC |
| | | | ACACCACCCGTGCTGGACTCCGATGGCTCTTTCGCCCTGGTGTCTAAGC |
| | | | TGACCGTGGACAAGAGCAGGTGGCAGCAGGGCAACGTGTTTTCCTGCT |
| | | | CTGTGATGCACGAGGCCCTGCACAATCACTACACCCAGAAGAGCCTGA |
| | | | GCTTAAGCCCAGGAGGCTCCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCCGAGGAGGATGGCATCACCT |
| | | | GGACACTGGATCAGAGCTCCGAGGTGCTGGGCAGCGGCAAGACCCTGA |
| | | | CAATCCAGGTGAAGGAGTTCGGCGACGCCGGACAGTACACATGTCACA |
| | | | AGGGCGGCGAGGTGCTGTCCCACTCTCTGCTGCTGCTGCACAAGAAGG |
| | | | AGGACGGCATCTGGTCTACAGACATCCTGAAGGATCAGAAGGAGCCTA |
| | | | AGAACAAGACCTTCCTGCGGTGCGAGGCCAAGAATTATAGCGGCAGAT |
| | | | TCACCTGTTGGTGGCTGACCACAATCAGCACCGATCTGACATTTTCCGT |
| | | | GAAGTCTAGCAGGGGCTCCTCTGACCCACAGGGAGTGACATGCGGAGC |
| | | | CGCCACCCTGTCCGCCGAGCGGGTGAGAGGCGATAACAAGGAGTACGA |
| | | | GTATTCCGTGGAGTGCCAGGAGGACTCTGCCTGTCCCGCCGCCGAGGAG |
| | | | AGCCTGCCTATCGAAGTGATGGTGGATGCCGTGCACAAGCTGAAGTAC |
| | | | GAGAATTATACAAGCTCCTTCTTTATCAGGGACATCATCAAGCCAGATC |
| | | | CCCCTAAGAACCTGCAGCTGAAGCCCCTGAAGAACAGCAGACAGGTGG |
| | | | AGGTGAGCTGGGAGTACCCTGACACCTGGTCCACACCACACTCTTATTT |
| | | | CAGCCTGACCTTTTGCGTGCAGGTGCAGGGCAAGTCTAAGAGGGAGAA |
| | | | GAAGGACCGCGTGTTCACCGATAAGACAAGCGCCACCGTGATCTGTAG |
| | | | AAAGAACGCCAGCATCTCCGTGAGGGCCCAGGATCGCTACTATTCTAGC |
| | | | TCCTGGTCCGAGTGGGCCTCTGTGCCCTGCAGCGGAGGAGGAGGCTCCG |
| | | | GAGGAGGAGGCTCTGGCGGCGGCGGCAGCGGAGGCGGCGGCTCCCGG |
| | | | GCCGTGCCAGGAGGCTCTAGCCCCGCCTGGACACAGTGCCAGCAGCTG |
| | | | AGCCAGAAGCTGTGCACCCTGGCCTGGTCCGCCCACCCTCTGGTGGGAC |
| | | | ACATGGACCTGAGAGAGGAGGGCGATGAGGAGACCACAAACGACGTG |
| | | | CCTCACATCCAGTGCGGCGACGGCTGTGATCCACAGGGCCTGAGGGAC |
| | | | AATTCCCAGTTCTGTCTGCAGCGCATCCACCAGGGCCTGATCTTCTACG |
| | | | AGAAGCTGCTGGGCTCTGATATCTTTACAGGCGAGCCCTCTCTGCTGCC |
| | | | TGACAGCCCAGTGGGACAGCTGCACGCCTCCCTGCTGGGCCTGTCTCAG |
| | | | CTGCTGCAGCCAGAGGGACACCACTGGGAGACCCAGCAGATCCCTTCT |
| | | | CTGAGCCCATCCCAGCCTTGGCAGCGGCTGCTGCTGCGGTTCAAGATCC |
| | | | TGCGGAGCCTGCAGGCCTTCGTGGCAGTGGCAGCAAGAGTGTTTGCAC |
| | | | ATGGAGCCGCCACCCTGTCCCCC |
| 115 | 18939 | Full nt | CAGGTGCAGCTGGTGGAGTCCGGCGGCGGCGTGGTGCAGCCAGGCCGG |
| | | | TCCCTGAGACTGTCTTGCGCAGCCAGCGGCTTCACCTTCAGCTCCTACG |
| | | | GCATGCACTGGGTGCGGCAGGCCCCCGGCAAGGGACTGGAGTGGGTGG |
| | | | CCTTCATCAGATATGACGGCAGCAACAAGTACTATGCCGATTCCGTGAA |
| | | | GGGCAGGTTTACCATCTCCCGCGACAACTCTAAGAATACACTGTACCTG |
| | | | CAGATGAACTCTCTGCGGGCCGAGGACACCGCCGTGTACTATTGTAAGA |
| | | | CACACGGCAGCCACGATAATTGGGGCCAGGGCACCATGGTGACAGTGT |
| | | | CTAGCGCTAGCACTAAGGGGCCTTCAGTGTTTCCACTGGCACCCAGTTC |
| | | | AAAATCAACAAGCGGAGGAACTGCCGCTCTGGGATGTCTGGTGAAGGA |
| | | | CTATTTCCCAGAGCCAGTCACCGTGAGCTGGAACTCCGGCGCACTGACT |
| | | | TCCGGAGTCCACACCTTTCCAGCCGTGCTGCAGAGCTCCGGACTGTACT |
| | | | CTCTGTCTAGTGTGGTCACAGTGCCTTCAAGCTCCCTGGGCACCCAGAC |
| | | | ATATATCTGCAACGTGAATCACAAGCCTAGTAATACTAAGGTCGACAA |
| | | | ACGCGTGGAACCAAAGAGCTGTGATAAAACTCATACCTGCCCCCCTTGT |
| | | | CCGGCGCCAGAGGCAGCAGGAGGACCAAGCGTGTTCCTGTTTCCACCC |
| | | | AAGCCCAAAGACACCCTGATGATTAGCCGAACCCCTGAAGTCACATGC |
| | | | GTGGTCGTGTCCGTGTCTCACGAGGACCCAGAAGTCAAGTTCAACTGGT |
| | | | ACGTGGATGGCGTCGAGGTGCATAATGCCAAGACAAAACCCCGGGAGG |
| | | | AACAGTACAACAGCACCTATAGAGTCGTGTCCGTCCTGACAGTGCTGCA |
| | | | CCAGGATTGGCTGAACGGCAAGGAATATAAGTGCAAAGTGTCCAATAA |
| | | | GGCCCTGCCCGCTCCTATCGAGAAAACCATTTCTAAGGCAAAAGGCCA |
| | | | GCCTCGCGAACCACAGGTCTACGTGCTGCCTCCATCCCGGGACGAGCTG |
| | | | ACAAAGAACCAGGTCTCTCTGCTGTGCCTGGTGAAAGGCTTCTATCCAT |
| | | | CAGATATTGCTGTGGAGTGGGAAAGCAATGGGCAGCCCGAGAACAATT |
| | | | ACCTGACTTGGCCCCCTGTGCTGGACTCTGATGGGAGTTTCTTTCTGTAT |
| | | | TCTAAGCTGACCGTGGATAAAAGTAGGTGGCAGCAGGGAAATGTCTTT |
| | | | AGTTGTTCAGTGATGCATGAAGCCCTGCATAACCACTACACCCAGAAAA |
| | | | GCCTGTCCCTGTCCCCCGGA |
| 116 | 18940 | Full nt | CAGTCCGTGCTGACCCAGCCACCTAGCGTGTCCGGAGCCCCCGGCCAGA |
| | | | GGGTGACAATCTCTTGCAGCGGCTCCCGCTCTAACATCGGCTCTAATAC |
| | | | CGTGAAGTGGTACCAGCAGCTGCCAGGCACAGCCCCCAAGCTGCTGAT |
| | | | CTACTATAACGACCAGCGGCCTAGCGGCGTGCCAGATAGATTCAGCGG |
| | | | CTCCAAGTCTGGCACCAGCGCCTCCCTGGCCATCACAGGACTGCAGGCA |
| | | | GAGGACGAGGCAGATTACTATTGTCAGTCTTACGACCGGTATACCCACC |
| | | | CCGCCCTGCTGTTTGGAACCGGAACAAAGGTGACAGTGCTGGGCCAGC |
| | | | CTAAGGCGGCGCCCAGCGTGACTCTGTTTCCACCCAGCTCCGAGGAACT |
| | | | GCAGGCCAATAAGGCTACCCTGGTCTGTCTGATTTCCGACTTCTACCCC |
| | | | GGGGCTGTGACAGTCGCATGGAAGGCCGATTCTAGTCCTGTGAAAGCA |
| | | | GGAGTCGAGACCACAACTCCATCAAAGCAGAGCAACAACAAGTACGCA |
| | | | GCCTCAAGCTATCTGTCTCTGACACCTGAACAGTGGAAAAGCCACCGGT |
| | | | CTTATAGTTGTCAGGTGACTCACGAGGGCTCAACAGTGGAAAAGACAG |
| | | | TCGCACCCGCAGAATGCTCA |
| 117 | 18942 | Full nt | CAGGTGCAGCTGGTGGAGAGCGGCGGCGGCGTGGTGCAGCCTGGCCGG |
| | | | TCCCTGAGACTGTCTTGCGCAGCCAGCGGCTTCACCTTTAGCTCCTACG |
| | | | GCATGCACTGGGTGCGGCAGGCCCCTGGCAAGGGACTGGAGTGGGTGG |
| | | | CCTTCATCAGATATGACGGCTCTAACAAGTACTATGCCGATAGCGTGAA |
| | | | GGGCAGGTTTACCATCTCCCGCGACAACTCTAAGAATACACTGTACCTG |
| | | | CAGATGAACTCCCTGCGGGCAGAGGACACCGCCGTGTACTATTGCAAG |
| | | | ACACACGGCTCTCACGATAATTGGGGCCAGGGCACCATGGTGACAGTG |
| | | | TCTAGCGGAGGAGGAGGCAGCGGAGGAGGAGGCTCCGGCGGCGGCGG |
| | | | CAGCCAGTCCGTGCTGACCCAGCCACCTTCTGTGAGCGGAGCCCCCGGA |
| | | | CAGAGGGTGACAATCTCCTGTTCTGGCAGCCGCTCCAACATCGGCAGCA |
| | | | ATACCGTGAAGTGGTATCAGCAGCTGCCTGGCACAGCCCCAAAGCTGCT |
| | | | GATCTACTATAATGACCAGAGACCCTCCGGCGTGCCTGATAGATTCTCT |
| | | | GGCAGCAAGTCCGGCACCTCTGCCAGCCTGGCCATCACAGGACTGCAG |
| | | | GCAGAGGACGAGGCAGATTACTATTGTCAGTCCTACGACAGATATACC |
| | | | CACCCAGCCCTGCTGTTTGGCACCGGCACAAAGGTGACAGTGCTGGCCG |
| | | | CCGAGCCCAAGTCCTCTGATAAGACCCACACATGCCCACCCTGTCCGGC |
| | | | GCCAGAGGCAGCAGGAGGACCAAGCGTGTTCCTGTTTCCACCCAAGCC |
| | | | CAAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAACCACAGGTCTACGTGCTGCCTCCATCCCGGGACGAGCTGACAAAG |
| | | | AACCAGGTCTCTCTGCTGTGCCTGGTGAAAGGCTTCTATCCATCAGATA |
| | | | TTGCTGTGGAGTGGGAAAGCAATGGGCAGCCCGAGAACAATTACCTGA |
| | | | CTTGGCCCCCTGTGCTGGACTCTGATGGGAGTTTCTTTCTGTATTCTAAG |
| | | | CTGACCGTGGATAAAAGTAGGTGGCAGCAGGGAAATGTCTTTAGTTGTT |
| | | | CAGTGATGCATGAAGCCCTGCATAACCACTACACCCAGAAAAGCCTGT |
| | | | CCCTGTCCCCCGGA |
| 118 | 18943 | Full nt | CAGTCCGTGCTGACCCAGCCACCTAGCGTGTCCGGAGCCCCCGGCCAGA |
| | | | GGGTGACAATCTCTTGCAGCGGCTCCCGCTCTAACATCGGCTCTAATAC |
| | | | CGTGAAGTGGTACCAGCAGCTGCCTGGCACAGCCCCAAAGCTGCTGAT |
| | | | CTACTATAACGACCAGAGACCCAGCGGCGTGCCTGATAGATTCAGCGG |
| | | | CTCCAAGTCTGGCACCAGCGCCTCCCTGGCCATCACAGGACTGCAGGCA |
| | | | GAGGACGAGGCAGATTACTATTGCCAGTCCTACGACCGGTATACCCACC |
| | | | CTGCCCTGCTGTTTGGCACCGGCACAAAGGTGACAGTGCTGGGCGGAG |
| | | | GAGGCTCCGGAGGAGGAGGCTCTGGCGGCGGCGGCAGCCAGGTGCAGC |
| | | | TGGTGGAGAGCGGCGGCGGCGTGGTGCAGCCAGGCCGGTCTCTGAGAC |
| | | | TGAGCTGTGCCGCCTCCGGCTTCACCTTTAGCTCCTACGGCATGCACTG |
| | | | GGTGCGGCAGGCCCCCGGCAAGGGACTGGAGTGGGTGGCCTTCATCAG |
| | | | ATATGACGGCTCCAATAAGTACTATGCCGATTCTGTGAAGGGCAGGTTT |
| | | | ACCATCAGCCGGGACAACAGCAAGAATACACTGTATCTGCAGATGAAC |
| | | | AGCCTGAGAGCCGAGGACACCGCCGTGTACTATTGTAAGACACACGGC |
| | | | TCCCACGATAATTGGGGCCAGGGCACCATGGTGACAGTGTCTAGCGCC |
| | | | GCCGAGCCCAAGTCCTCTGATAAGACCCACACATGCCCACCCTGTCCGG |
| | | | CGCCAGAGGCAGCAGGAGGACCAAGCGTGTTCCTGTTTCCACCCAAGC |
| | | | CCAAAGACACCCTGATGATTAGCCGAACCCCTGAAGTCACATGCGTGGT |
| | | | CGTGTCCGTGTCTCACGAGGACCCAGAAGTCAAGTTCAACTGGTACGTG |
| | | | GATGGCGTCGAGGTGCATAATGCCAAGACAAAACCCCGGGAGGAACAG |
| | | | TACAACAGCACCTATAGAGTCGTGTCCGTCCTGACAGTGCTGCACCAGG |
| | | | ATTGGCTGAACGGCAAGGAATATAAGTGCAAAGTGTCCAATAAGGCCC |
| | | | TGCCCGCTCCTATCGAGAAAACCATTTCTAAGGCAAAAGGCCAGCCTCG |
| | | | CGAACCACAGGTCTACGTGCTGCCTCCATCCCGGGACGAGCTGACAAA |
| | | | GAACCAGGTCTCTCTGCTGTGCCTGGTGAAAGGCTTCTATCCATCAGAT |
| | | | ATTGCTGTGGAGTGGGAAAGCAATGGGCAGCCCGAGAACAATTACCTG |
| | | | ACTTGGCCCCCTGTGCTGGACTCTGATGGGAGTTTCTTTCTGTATTCTAA |
| | | | GCTGACCGTGGATAAAAGTAGGTGGCAGCAGGGAAATGTCTTTAGTTG |
| | | | TTCAGTGATGCATGAAGCCCTGCATAACCACTACACCCAGAAAAGCCTG |
| | | | TCCCTGTCCCCCGGA |
| 119 | 18953 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCAGGAGGAGGAGGAGGCTCTCTGAGCGGCAGGAGCGATA |
| | | | ATCATGGAGGAGGAGGCAGCGGAGGAGGAGGCTCCGGAGGCGGCGGC |
| | | | TCTGGCGGCGGCGGCAGCAAGATCGACGCCTGCAAGCGCGGCGATGTG |
| | | | ACAGTGAAGCCTAGCCACGTGATCCTGCTGGGCTCCACCGTGAATATCA |
| | | | CATGCTCTCTGAAGCCACGGCAGGGCTGTTTCCACTACTCCCGGAGAAA |
| | | | CAAGCTGATCCTGTATAAGTTCGACAGGCGCATCAACTTTCACCACGGC |
| | | | CACTCTCTGAATAGCCAGGTGACCGGACTGCCACTGGGCACCACACTGT |
| | | | TCGTGTGCAAGCTGGCCTGTATCAATAGCGATGAGATCCAGATCTGTGG |
| | | | AGCCGAGATCTTTGTGGGAGTGGCCCCCGAGCAGCCTCAGAACCTGTCC |
| | | | TGCATCCAGAAGGGAGAGCAGGGAACCGTGGCATGTACATGGGAGCGG |
| | | | GGCAGAGACACCCACCTGTACACCGAGTATACACTGCAGCTGAGCGGC |
| | | | CCCAAGAACCTGACATGGCAGAAGCAGTGCAAGGATATCTACTGTGAC |
| | | | TATCTGGATTTCGGCATCAACCTGACCCCAGAGTCCCCCGAGTCTAACT |
| | | | TCACCGCCAAGGTGACAGCCGTGAACTCTCTGGGCAGCTCCTCTAGCCT |
| | | | GCCCAGCACCTTCACATTTCTGGACATCGTGCGCCCTCTGCCTCCATGG |
| | | | GATATCCGGATCAAGTTTCAGAAGGCCTCCGTGTCTAGATGCACACTGT |
| | | | ACTGGAGGGACGAGGGCCTGGTGCTGCTGAACAGGCTGCGCTATAGAC |
| | | | CCAGCAATTCCAGACTGTGGAACATGGTGAATGTGACCAAGGCCAAGG |
| | | | GCAGGCACGACCTGCTGGATCTGAAGCCTTTCACAGAGTACGAGTTTCA |
| | | | GATCTCCTCTAAGCTGCACCTGTATAAGGGCTCTTGGAGCGATTGGTCC |
| | | | GAGTCTCTGAGGGCACAGACCCCTGAGGAGGAGCCA |
| 120 | 18954 | Full nt | AAGATCGACGCCTGCAAGCGGGGCGATGTGACAGTGAAGCCATCCCAC |
| | | | GTGATCCTGCTGGGCTCTACCGTGAATATCACATGCAGCCTGAAGCCAC |
| | | | GGCAGGGCTGTTTTCACTACTCCCGGAGAAACAAGCTGATCCTGTATAA |
| | | | GTTCGACAGGCGCATCAACTTTCACCACGGCCACAGCCTGAACAGCCA |
| | | | GGTGACCGGACTGCCCCTGGGCACCACACTGTTCGTGTGCAAGCTGGCC |
| | | | TGTATCAATTCTGATGAGATCCAGATCTGTGGAGCCGAGATCTTTGTGG |
| | | | GCGTGGCCCCTGAGCAGCCACAGAACCTGAGCTGCATCCAGAAGGGAG |
| | | | AGCAGGGAACCGTGGCATGTACATGGGAGCGGGGCAGAGACACCCACC |
| | | | TGTACACCGAGTATACACTGCAGCTGAGCGGCCCTAAGAATCTGACATG |
| | | | GCAGAAGCAGTGCAAGGATATCTACTGTGACTATCTGGATTTCGGCATC |
| | | | AACCTGACCCCCGAGTCTCCTGAGAGCAACTTCACCGCCAAGGTGACA |
| | | | GCCGTGAACAGCCTGGGCAGCTCCTCTAGCCTGCCTTCCACCTTCACAT |
| | | | TTCTGGACATCGTGAGACCACTGCCCCCTTGGGATATCAGGATCAAGTT |
| | | | CCAGAAGGCCTCTGTGAGCAGATGCACACTGTACTGGAGGGACGAGGG |
| | | | CCTGGTGCTGCTGAACAGGCTGCGCTATAGACCCTCCAATTCTCGCCTG |
| | | | TGGAACATGGTGAATGTGACCAAGGCCAAGGGCAGACACGACCTGCTG |
| | | | GATCTGAAGCCTTTCACAGAGTACGAGTTTCAGATCTCCTCTAAGCTGC |
| | | | ACCTGTATAAGGGCAGCTGGTCCGATTGGTCTGAGAGCCTGAGAGCCC |
| | | | AGACCCCAGAGGAGGAGCCAGGAGGAGGAGGCTCCGGCGGAGGAGGC |
| | | | TCCCTGTCTGGCAGGTCCGACAACCACGGAGGAGGAGGCTCTGAGCCC |
| | | | AAGAGCTCCGATAAGACCCACACATGCCCACCCTGTCCGGCGCCAGAG |
| | | | GCAGCAGGAGGACCAAGCGTGTTCCTGTTTCCACCCAAGCCCAAAGAC |
| | | | ACCCTGATGATTAGCCGAACCCCTGAAGTCACATGCGTGGTCGTGTCCG |
| | | | TGTCTCACGAGGACCCAGAAGTCAAGTTCAACTGGTACGTGGATGGCGT |
| | | | CGAGGTGCATAATGCCAAGACAAAACCCCGGGAGGAACAGTACAACAG |
| | | | CACCTATAGAGTCGTGTCCGTCCTGACAGTGCTGCACCAGGATTGGCTG |
| | | | AACGGCAAGGAATATAAGTGCAAAGTGTCCAATAAGGCCCTGCCCGCT |
| | | | CCTATCGAGAAAACCATTTCTAAGGCAAAAGGCCAGCCTCGCGAACCA |
| | | | CAGGTCTACGTGCTGCCTCCATCCCGGGACGAGCTGACAAAGAACCAG |
| | | | GTCTCTCTGCTGTGCCTGGTGAAAGGCTTCTATCCATCAGATATTGCTGT |
| | | | GGAGTGGGAAAGCAATGGGCAGCCCGAGAACAATTACCTGACTTGGCC |
| | | | CCCTGTGCTGGACTCTGATGGGAGTTTCTTTCTGTATTCTAAGCTGACCG |
| | | | TGGATAAAAGTAGGTGGCAGCAGGGAAATGTCTTTAGTTGTTCAGTGAT |
| | | | GCATGAAGCCCTGCATAACCACTACACCCAGAAAAGCCTGTCCCTGTCC |
| | | | CCCGGA |
| 121 | 18956 | Full nt | AAGATCGACGCATGCAAGAGGGGCGATGTGACAGTGAAGCCTTCTCAC |
| | | | GTGATCCTGCTGGGCAGCACCGTGAACATCACATGCTCCCTGAAGCCCA |
| | | | GACAGGGCTGTTTTCACTACTCCCGGAGAAATAAGCTGATCCTGTATAA |
| | | | GTTCGATAGGCGCATCAACTTTCACCACGGCCACTCTCTGAATAGCCAG |
| | | | GTGACCGGACTGCCTCTGGGCACCACACTGTTCGTGTGCAAGCTGGCCT |
| | | | GTATCAACTCTGACGAGATCCAGATCTGTGGAGCCGAGATCTTTGTGGG |
| | | | CGTGGCCCCAGGAGGAGGAGGCAGCGGAGGAGGCGGCAGCCTGAGCG |
| | | | GCAGAAGCGATAACCATGGAGGAGGAGGCAGCAGAAATCTGCCAGTG |
| | | | GCCACACCAGACCCCGGAATGTTCCCTTGCCTGCACCACTCCCAGAACC |
| | | | TGCTGAGGGCCGTGTCTAATATGCTGCAGAAGGCCCGCCAGACCCTGG |
| | | | AGTTTTACCCATGTACAAGCGAGGAGATCGACCACGAGGATATCACCA |
| | | | AGGATAAGACCTCCACAGTGGAGGCATGCCTGCCACTGGAGCTGACAA |
| | | | AGAACGAGTCCTGTCTGAACAGCCGGGAGACCAGCTTCATCACAAACG |
| | | | GCTCCTGCCTGGCCTCTAGAAAGACCAGCTTTATGATGGCCCTGTGCCT |
| | | | GAGCTCCATCTACGAGGACCTGAAGATGTATCAGGTGGAGTTCAAGAC |
| | | | AATGAACGCCAAGCTGCTGATGGACCCCAAGCGGCAGATCTTTCTGGAT |
| | | | CAGAATATGCTGGCCGTGATCGACGAGCTGATGCAGGCCCTGAACTTCA |
| | | | ATAGCGAGACCGTGCCTCAGAAGTCTAGCCTGGAGGAGCCAGATTTCT |
| | | | ACAAGACAAAGATCAAGCTGTGCATCCTGCTGCACGCCTTTCGGATCAG |
| | | | AGCCGTGACCATCGACAGAGTGATGTCCTACCTGAACGCCAGCGGCGG |
| | | | CGGCGGCAGCGGCGGAGGCGGCTCCGAGCCTAAGTCCTCTGATAAGAC |
| | | | CCACACATGCCCCCCTTGTCCGGCGCCAGAGGCTGCAGGAGGACCAAG |
| | | | CGTGTTCCTGTTTCCACCCAAGCCTAAAGACACACTGATGATTTCCCGA |
| | | | ACCCCCGAAGTCACATGCGTGGTCGTGTCTGTGAGTCACGAGGACCCTG |
| | | | AAGTCAAGTTCAACTGGTACGTGGATGGCGTCGAGGTGCATAATGCCA |
| | | | AGACTAAACCTAGGGAGGAACAGTACAACTCAACCTATCGCGTCGTGA |
| | | | GCGTCCTGACAGTGCTGCACCAGGATTGGCTGAACGGCAAAGAATATA |
| | | | AGTGCAAAGTGAGCAATAAGGCCCTGCCCGCTCCTATCGAGAAAACCA |
| | | | TTTCCAAGGCTAAAGGGCAGCCTCGCGAACCACAGGTCTACGTGTATCC |
| | | | TCCAAGCCGGGACGAGCTGACAAAGAACCAGGTCTCCCTGACTTGTCTG |
| | | | GTGAAAGGGTTTTACCCTAGTGATATCGCTGTGGAGTGGGAATCAAATG |
| | | | GACAGCCAGAGAACAATTATAAGACTACCCCCCCTGTGCTGGACAGTG |
| | | | ATGGGTCATTCGCACTGGTCTCCAAGCTGACAGTGGACAAATCTCGGTG |
| | | | GCAGCAGGGAAATGTCTTTTCATGTAGCGTGATGCATGAAGCACTGCAC |
| | | | AACCATTACACCCAGAAGTCACTGTCACTGTCACCAGGA |
| 122 | 18957 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCCGGAGGGTCTGCTGATGGGGGCATTTGGGAACTGAAGA |
| | | | AAGATGTCTATGTCGTGGAGCTGGACTGGTATCCTGACGCACCTGGGGA |
| | | | GATGGTGGTGCTGACCTGCGACACACCTGAGGAGGATGGCATCACCTG |
| | | | GACACTGGATCAGAGCTCCGAGGTGCTGGGCAGCGGCAAGACCCTGAC |
| | | | AATCCAGGTGAAGGAGTTCGGCGACGCCGGACAGTACACCTGTCACAA |
| | | | GGGAGGAGAGGTGCTGAGCCACTCCCTGCTGCTGCTGCACAAGAAGGA |
| | | | GGACGGCATCTGGTCTACAGACATCCTGAAGGATCAGAAGGAGCCCAA |
| | | | GAACAAGACCTTCCTGCGGTGCGAGGCCAAGAATTATAGCGGCAGATT |
| | | | CACCTGTTGGTGGCTGACCACAATCTCTACCGACCTGACCTTCAGCGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATAGCGTGGAGTGCCAGGAGGACTCCGCCTGTCCCGCCGCCGAGGAG |
| | | | TCCCTGCCTATCGAAGTGATGGTGGATGCCGTGCACAAGCTGAAGTACG |
| | | | AGAATTATACAAGCTCCTTCTTTATCCGGGACATCATCAAGCCTGATCC |
| | | | CCCTAAGAACCTGCAGCTGAAGCCCCTGAAGAACAGCAGACAGGTGGA |
| | | | GGTGTCTTGGGAGTACCCTGACACCTGGTCCACACCACACAGCTATTTC |
| | | | TCCCTGACCTTTTGCGTGCAGGTGCAGGGCAAGTCCAAGAGGGAGAAG |
| | | | AAGGACCGCGTGTTCACCGATAAGACATCTGCCACCGTGATCTGTCGGA |
| | | | AGAACGCCTCTATCAGCGTGCGGGCCCAGGATAGATACTATTCTAGCTC |
| | | | CTGGTCCGAGTGGGCCTCTGTGCCATGCAGTGGAGGAGGAGGCTCCGG |
| | | | AGGAGGAGGCTCTGGAGGAGGAGGCAGCAGAAATCTGCCAGTGGCCAC |
| | | | CCCAGACCCCGGAATGTTCCCTTGCCTGCACCACTCTCAGAACCTGCTG |
| | | | AGGGCCGTGAGCAATATGCTGCAGAAGGCCCGCCAGACACTGGAGTTT |
| | | | TACCCTTGTACCAGCGAGGAGATCGACCACGAGGATATCACAAAGGAT |
| | | | AAGACCTCCACAGTGGAGGCCTGCCTGCCACTGGAGCTGACCAAGAAC |
| | | | GAGTCCTGTCTGAACAGCCGGGAGACAAGCTTCATCACCAACGGCTCCT |
| | | | GCCTGGCCTCTAGAAAGACAAGCTTTATGATGGCCCTGTGCCTGAGCAG |
| | | | CATCTACGAGGACCTGAAGATGTATCAGGTGGAGTTCAAGACCATGAA |
| | | | CGCCAAGCTGCTGATGGACCCCAAGCGGCAGATCTTTCTGGATCAGAAT |
| | | | ATGCTGGCCGTGATCGACGAGCTGATGCAGGCCCTGAACTTCAATAGCG |
| | | | AGACAGTGCCACAGAAGTCCTCTCTGGAGGAGCCCGATTTCTACAAGA |
| | | | CCAAGATCAAGCTGTGCATCCTGCTGCACGCCTTCCGGATCAGAGCCGT |
| | | | GACAATCGATAGAGTGATGTCCTATCTGAACGCCTCTGGAGGAGGAGG |
| | | | CTCCCTGTCTGGCCGCAGCGACAATCATGGAGGAGGAGGCAGCGGCGG |
| | | | CGGAGGCTCCAAGATCGACGCCTGTAAGAGGGGCGATGTGACCGTGAA |
| | | | GCCATCTCACGTGATCCTGCTGGGCAGCACAGTGAACATCACCTGCTCC |
| | | | CTGAAGCCCAGACAGGGCTGTTTCCACTACTCCCGGAGAAATAAGCTG |
| | | | ATCCTGTATAAGTTCGACAGGCGCATCAACTTTCACCACGGCCACTCTC |
| | | | TGAATAGCCAGGTGACCGGCCTGCCCCTGGGCACCACACTGTTCGTGTG |
| | | | CAAGCTGGCCTGTATCAATAGTGACGAGATTCAGATTTGTGGGGCAGA |
| | | | GATTTTTGTGGGGGTCGCTCCC |
| 123 | 21415 | Full nt | ATCTGGGAGCTGAAGAAGGACGTGTACGTGGTGGAGCTGGACTGGTAC |
| | | | CCTGATGCCCCAGGCGAGATGGTGGTGCTGACCTGCGACACACCCGAG |
| | | | GAGGATGGCATCACCTGGACACTGGACCAGAGCTCCGAGGTGCTGGGC |
| | | | AGCGGCAAGACCCTGACAATCCAGGTGAAGGAGTTCGGCGATGCCGGA |
| | | | CAGTACACCTGTCACAAGGGAGGAGAGGTGCTGAGCCACTCCCTGCTG |
| | | | CTGCTGCACAAGAAGGAGGATGGCATCTGGTCCACAGACATCCTGAAG |
| | | | GATCAGAAGGAGCCAAAGAACAAGACCTTCCTGCGGTGCGAGGCCAAG |
| | | | AATTATAGCGGCAGATTCACCTGTTGGTGGCTGACCACAATCTCCACCG |
| | | | ACCTGACATTTTCTGTGAAGTCTAGCAGGGGCTCCTCTGATCCCCAGGG |
| | | | AGTGACATGCGGAGCCGCCACCCTGTCCGCCGAGCGGGTGAGAGGCGA |
| | | | CAACAAGGAGTACGAGTATTCTGTGGAGTGCCAGGAGGATAGCGCCTG |
| | | | TCCCGCCGCCGAGGAGAGCCTGCCTATCGAAGTGATGGTGGACGCCGT |
| | | | GCACAAGCTGAAGTACGAGAATTATACAAGCTCCTTCTTTATCCGGGAC |
| | | | ATCATCAAGCCAGATCCCCCTAAGAACCTGCAGCTGAAGCCCCTGAAG |
| | | | AATAGCAGACAGGTGGAGGTGTCCTGGGAGTACCCTGATACCTGGAGC |
| | | | ACACCACACTCCTATTTCTCTCTGACCTTTTGCGTGCAGGTGCAGGGCA |
| | | | AGTCTAAGAGGGAGAAGAAGGACCGCGTGTTTACCGATAAGACAAGCG |
| | | | CCACCGTGATCTGTAGGAAGAACGCCTCTATCAGCGTGCGGGCACAGG |
| | | | ACCGGTACTATTCTAGCTCCTGGAGCGAGTGGGCCTCCGTGCCTTGCTC |
| | | | TGGAGGAGGAGGCAGCGGCGGAGGAGGCTCCATGAGCGGGCGGAGCG |
| | | | CCAACGCAGGGGGCGGCGGCTCTGGCGGCGGCGGCAGCGGAGGCGGC |
| | | | GGCAGCCAGTCCGTGCTGACACAGCCACCATCTGTGAGCGGAGCCCCC |
| | | | GGACAGAGGGTGACCATCTCCTGTTCTGGCAGCCGCTCCAACATCGGCT |
| | | | CCAATACAGTGAAGTGGTATCAGCAGCTGCCAGGAACCGCCCCTAAGC |
| | | | TGCTGATCTACTATAACGACCAGAGGCCAAGCGGAGTGCCAGATCGCTT |
| | | | CTCTGGCAGCAAGTCCGGCACATCTGCCAGCCTGGCCATCACCGGACTG |
| | | | CAGGCAGAGGACGAGGCCGATTACTATTGCCAGTCCTACGATCGGTAT |
| | | | ACACACCCTGCCCTGCTGTTTGGCACCGGCACAAAGGTGACCGTGCTGG |
| | | | GCGGAGGAGGCTCCGGAGGAGGAGGCTCTGGAGGAGGCGGCAGCCAG |
| | | | GTGCAGCTGGTGGAGAGCGGCGGCGGCGTGGTGCAGCCCGGCAGGAGC |
| | | | CTGCGCCTGTCCTGTGCAGCCTCTGGCTTCACCTTTTCTAGCTACGGCAT |
| | | | GCACTGGGTGCGGCAGGCCCCTGGCAAGGGACTGGAGTGGGTGGCCTT |
| | | | CATCAGATATGACGGCTCCAATAAGTACTATGCCGATTCTGTGAAGGGC |
| | | | CGGTTTACAATCAGCAGAGATAACTCCAAGAATACCCTGTACCTGCAGA |
| | | | TGAACTCCCTGAGAGCCGAGGACACAGCCGTGTACTATTGTAAGACCC |
| | | | ACGGCTCTCACGATAATTGGGGCCAGGGCACAATGGTGACCGTGTCCTC |
| | | | T |
| 124 | 21416 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCAGGCCGGAACCTGCCCGTGGCCACCCCAGATCCCGGCAT |
| | | | GTTCCCCTGCCTGCACCACTCTCAGAACCTGCTGAGGGCCGTGAGCAAT |
| | | | ATGCTGCAGAAGGCCCGCCAGACCCTGGAGTTTTACCCCTGTACATCCG |
| | | | AGGAGATCGACCACGAGGATATCACCAAGGACAAGACCTCTACAGTGG |
| | | | AGGCCTGCCTGCCTCTGGAGCTGACAAAGAACGAGTCTTGTCTGAATAG |
| | | | CCGGGAGACCTCCTTCATCACAAATGGCTCTTGCCTGGCCAGCAGAAAG |
| | | | ACCTCCTTTATGATGGCCCTGTGCCTGAGCTCCATCTACGAGGATCTGA |
| | | | AGATGTATCAGGTGGAGTTCAAGACAATGAACGCCAAGCTGCTGATGG |
| | | | ACCCTAAGCGGCAGATCTTTCTGGATCAGAATATGCTGGCCGTGATCGA |
| | | | CGAGCTGATGCAGGCCCTGAACTTCAATAGCGAGACCGTGCCTCAGAA |
| | | | GTCTAGCCTGGAGGAGCCAGATTTCTACAAGACAAAGATCAAGCTGTG |
| | | | CATCCTGCTGCACGCCTTTCGGATCAGAGCCGTGACCATCGACAGAGTG |
| | | | ATGTCCTATCTGAACGCCTCTGGAGGAGGAGGCAGCGGAGGAGGCGGC |
| | | | TCTATGAGCGGGCGGAGCGCCAACGCAGGGGGAGGAGGCTCCGGCGGA |
| | | | GGAGGCTCTGGCGGCGGCGGCTCCCAGTCTGTGCTGACCCAGCCACCTA |
| | | | GCGTGTCCGGAGCCCCCGGCCAGCGGGTGACAATCTCTTGTAGCGGCTC |
| | | | CAGATCTAACATCGGCAGCAATACCGTGAAGTGGTATCAGCAGCTGCCT |
| | | | GGCACAGCCCCAAAGCTGCTGATCTACTATAACGATCAGAGGCCCTCCG |
| | | | GCGTGCCTGACCGCTTCAGCGGCTCCAAGTCTGGCACCAGCGCCTCCCT |
| | | | GGCCATCACAGGCCTGCAGGCAGAGGACGAGGCAGATTACTATTGCCA |
| | | | GAGCTACGATAGGTATACCCACCCAGCCCTGCTGTTTGGCACCGGCACA |
| | | | AAGGTGACAGTGCTGGGCGGCGGCGGCAGCGGAGGAGGAGGCTCCGG |
| | | | AGGCGGCGGCTCTCAGGTGCAGCTGGTGGAGTCCGGAGGAGGAGTGGT |
| | | | GCAGCCAGGCAGGTCTCTGCGCCTGAGCTGTGCAGCCTCCGGCTTCACC |
| | | | TTTTCCTCTTACGGCATGCACTGGGTGAGGCAGGCCCCCGGCAAGGGAC |
| | | | TGGAGTGGGTGGCCTTCATCCGCTATGATGGCAGCAATAAGTACTATGC |
| | | | CGACTCCGTGAAGGGCCGGTTTACCATCTCTAGAGACAACAGCAAGAA |
| | | | TACACTGTATCTGCAGATGAACAGCCTGCGCGCCGAGGATACCGCCGTG |
| | | | TACTATTGCAAGACACACGGCTCCCACGACAATTGGGGCCAGGGCACC |
| | | | ATGGTGACAGTGAGCTCC |
| 125 | 21417 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCAGGAGGAGGAGGAGGCAGCGGCGGAGGAGGCTCCATG |
| | | | AGCGGGCGGAGCGCCAACGCAGGGGGCGGCGGCAGCGGAGGCGGCGG |
| | | | CAGCCAGTCCGTGCTGACCCAGCCACCTTCTGTGAGCGGAGCCCCCGGA |
| | | | CAGAGGGTGACAATCTCCTGCTCTGGCAGCCGCTCCAACATCGGCTCCA |
| | | | ATACCGTGAAGTGGTATCAGCAGCTGCCAGGCACAGCCCCCAAGCTGC |
| | | | TGATCTACTATAATGACCAGCGGCCTAGCGGCGTGCCAGATAGATTCTC |
| | | | TGGCAGCAAGTCCGGCACCTCTGCCAGCCTGGCCATCACAGGCCTGCAG |
| | | | GCAGAGGACGAGGCAGATTACTATTGCCAGTCCTACGACCGCTATACCC |
| | | | ACCCTGCCCTGCTGTTTGGCACCGGCACAAAGGTGACAGTGCTGGGCGG |
| | | | CGGCGGCAGCGGCGGGGGAGGCTCCGGCGGCGGCGGCTCTCAGGTGCA |
| | | | GCTGGTGGAGAGCGGAGGAGGAGTGGTGCAGCCCGGCAGAAGCCTGCG |
| | | | GCTGAGCTGTGCAGCCAGCGGCTTCACCTTTAGCTCCTACGGCATGCAC |
| | | | TGGGTGCGGCAGGCCCCTGGCAAGGGACTGGAGTGGGTGGCCTTCATC |
| | | | AGATATGACGGCAGCAACAAGTACTATGCCGATTCCGTGAAGGGCAGG |
| | | | TTTACCATCTCCCGCGATAACTCTAAGAATACACTGTATCTGCAGATGA |
| | | | ACTCCCTGCGGGCAGAGGACACCGCCGTGTACTATTGTAAGACACACG |
| | | | GCTCTCACGATAATTGGGGCCAGGGCACCATGGTGACAGTGTCTAGC |
| 126 | 21418 | Full nt | CAGGTGCAGCTGGTGGAGAGCGGCGGCGGCGTGGTGCAGCCTGGCAGG |
| | | | TCTCTGCGCCTGAGCTGCGCAGCCTCCGGCTTCACCTTTAGCTCCTACGG |
| | | | CATGCACTGGGTGCGGCAGGCCCCTGGCAAGGGACTGGAGTGGGTGGC |
| | | | CTTCATCAGATATGACGGCTCCAACAAGTACTATGCCGATTCTGTGAAG |
| | | | GGCAGGTTTACAATCAGCCGGGACAACAGCAAGAATACCCTGTACCTG |
| | | | CAGATGAACTCCCTGAGGGCCGAGGACACAGCCGTGTACTATTGCAAG |
| | | | ACCCACGGCTCTCACGATAATTGGGGCCAGGGCACCATGGTGACAGTG |
| | | | TCTTCCGGAGGAGGAGGCTCCGGAGGAGGAGGCTCTGGCGGCGGCGGC |
| | | | TCTCAGAGCGTGCTGACACAGCCACCTTCCGTGTCTGGAGCCCCCGGAC |
| | | | AGCGGGTGACCATCAGCTGTTCCGGCTCTAGAAGCAACATCGGCAGCA |
| | | | ATACAGTGAAGTGGTATCAGCAGCTGCCAGGAACCGCCCCCAAGCTGC |
| | | | TGATCTACTATAACGACCAGAGGCCTTCCGGCGTGCCAGATCGCTTCTC |
| | | | CGGCTCTAAGAGCGGCACATCCGCCTCTCTGGCCATCACCGGACTGCAG |
| | | | GCAGAGGACGAGGCAGATTACTATTGTCAGAGCTACGACAGGTATACA |
| | | | CACCCTGCCCTGCTGTTTGGCACCGGCACAAAGGTGACCGTGCTGGGCG |
| | | | GAGGAGGCAGCGGCGGCGGAGGCTCCGGAGGCGGCGGCAGCATGAGC |
| | | | GGGCGGAGCGCCAACGCAGGGGGCGGCGGCTCTGGAGGAGGAGGCAG |
| | | | CATCTGGGAGCTGAAGAAGGACGTGTACGTGGTGGAGCTGGACTGGTA |
| | | | CCCTGATGCCCCTGGCGAGATGGTGGTGCTGACCTGCGACACACCAGA |
| | | | GGAGGATGGCATCACCTGGACACTGGACCAGTCCTCTGAGGTGCTGGG |
| | | | CAGCGGCAAGACCCTGACAATCCAGGTGAAGGAGTTCGGCGATGCCGG |
| | | | ACAGTACACCTGTCACAAGGGCGGCGAGGTGCTGTCTCACAGCCTGCTG |
| | | | CTGCTGCACAAGAAGGAGGACGGCATCTGGAGCACAGACATCCTGAAG |
| | | | GATCAGAAGGAGCCAAAGAACAAGACCTTCCTGAGGTGCGAGGCCAAG |
| | | | AATTATAGCGGCAGATTCACCTGTTGGTGGCTGACCACAATCAGCACCG |
| | | | ACCTGACATTTTCCGTGAAGAGCTCCCGGGGCTCTAGCGATCCCCAGGG |
| | | | AGTGACATGCGGAGCCGCCACCCTGAGCGCCGAGCGGGTGAGAGGCGA |
| | | | CAACAAGGAGTACGAGTATAGCGTGGAGTGCCAGGAGGATTCCGCCTG |
| | | | TCCAGCCGCCGAGGAGTCCCTGCCAATCGAAGTGATGGTGGACGCCGT |
| | | | GCACAAGCTGAAGTACGAGAATTATACCTCCTCTTTCTTTATCCGGGAC |
| | | | ATCATCAAGCCTGATCCACCCAAGAACCTGCAGCTGAAGCCCCTGAAG |
| | | | AACAGCAGACAGGTGGAGGTGAGCTGGGAGTACCCCGATACCTGGTCC |
| | | | ACACCTCACAGCTATTTCTCCCTGACCTTTTGCGTGCAGGTGCAGGGCA |
| | | | AGTCTAAGCGGGAGAAGAAGGACAGAGTGTTTACCGATAAGACAAGCG |
| | | | CCACCGTGATCTGTAGAAAGAACGCCAGCATCTCTGTGCGGGCACAGG |
| | | | ACCGGTACTATAGCTCCTCTTGGTCCGAGTGGGCCTCTGTGCCCTGCAG |
| | | | TGGCGGCGGCGGCTCCGGCGGAGGAGGCTCTGAGCCTAAGAGCTCCGA |
| | | | TAAGACCCACACATGCCCTCCATGTCCGGCGCCAGAGGCTGCAGGAGG |
| | | | ACCAAGCGTGTTCCTGTTTCCACCCAAGCCTAAAGACACACTGATGATT |
| | | | TCCCGAACCCCCGAAGTCACATGCGTGGTCGTGTCTGTGAGTCACGAGG |
| | | | ACCCTGAAGTCAAGTTCAACTGGTACGTGGATGGCGTCGAGGTGCATA |
| | | | ATGCCAAGACTAAACCTAGGGAGGAACAGTACAACTCAACCTATCGCG |
| | | | TCGTGAGCGTCCTGACAGTGCTGCACCAGGATTGGCTGAACGGCAAAG |
| | | | AATATAAGTGCAAAGTGAGCAATAAGGCCCTGCCCGCTCCTATCGAGA |
| | | | AAACCATTTCCAAGGCTAAAGGGCAGCCTCGCGAACCACAGGTCTACG |
| | | | TGTATCCTCCAAGCCGGGACGAGCTGACAAAGAACCAGGTCTCCCTGA |
| | | | CTTGTCTGGTGAAAGGGTTTTACCCTAGTGATATCGCTGTGGAGTGGGA |
| | | | ATCAAATGGACAGCCAGAGAACAATTATAAGACTACCCCCCCTGTGCT |
| | | | GGACAGTGATGGGTCATTCGCACTGGTCTCCAAGCTGACAGTGGACAA |
| | | | ATCTCGGTGGCAGCAGGGAAATGTCTTTTCATGTAGCGTGATGCATGAA |
| | | | GCACTGCACAACCATTACACCCAGAAGTCACTGTCACTGTCACCAGGA |
| 127 | 21419 | Full nt | AGAAACCTGCCCGTGGCCACCCCAGATCCCGGAATGTTTCCATGCCTGC |
| | | | ACCACTCTCAGAACCTGCTGCGGGCCGTGAGCAATATGCTGCAGAAGG |
| | | | CCAGACAGACCCTGGAGTTCTACCCCTGTACATCCGAGGAGATCGACCA |
| | | | CGAGGATATCACCAAGGACAAGACCTCTACAGTGGAGGCCTGCCTGCC |
| | | | TCTGGAGCTGACAAAGAACGAGTCTTGTCTGAATAGCAGGGAGACCTC |
| | | | CTTCATCACAAATGGCTCTTGCCTGGCCAGCCGCAAGACCTCCTTTATG |
| | | | ATGGCCCTGTGCCTGAGCTCCATCTACGAGGATCTGAAGATGTATCAGG |
| | | | TGGAGTTCAAGACAATGAACGCCAAGCTGCTGATGGACCCCAAGCGGC |
| | | | AGATCTTTCTGGATCAGAATATGCTGGCCGTGATCGACGAGCTGATGCA |
| | | | GGCCCTGAACTTTAATAGCGAGACCGTGCCTCAGAAGTCTAGCCTGGAG |
| | | | GAGCCAGATTTCTACAAGACAAAGATCAAGCTGTGCATCCTGCTGCACG |
| | | | CCTTCCGGATCAGAGCCGTGACCATCGACAGAGTGATGTCCTATCTGAA |
| | | | CGCCTCTGGAGGAGGAGGCAGCGGAGGAGGCGGCTCTATGAGCGGGCG |
| | | | GAGCGCCAACGCAGGGGGAGGAGGCTCCGGCGGAGGAGGCTCTGGCG |
| | | | GCGGCGGCTCCCAGTCTGTGCTGACCCAGCCACCTAGCGTGTCCGGAGC |
| | | | CCCCGGCCAGAGGGTGACAATCTCTTGTAGCGGCTCCCGCTCTAACATC |
| | | | GGCAGCAATACCGTGAAGTGGTATCAGCAGCTGCCTGGCACAGCCCCA |
| | | | AAGCTGCTGATCTACTATAACGATCAGAGACCCTCCGGCGTGCCTGACA |
| | | | GATTCAGCGGCTCCAAGTCTGGCACCAGCGCCTCCCTGGCCATCACAGG |
| | | | CCTGCAGGCAGAGGACGAGGCAGATTACTATTGCCAGAGCTACGATCG |
| | | | GTATACCCACCCTGCCCTGCTGTTCGGCACCGGCACAAAGGTGACAGTG |
| | | | CTGGGCGGCGGCGGCAGCGGAGGAGGAGGCTCCGGAGGCGGCGGCTCT |
| | | | CAGGTGCAGCTGGTGGAGTCCGGAGGAGGAGTGGTGCAGCCAGGCCGG |
| | | | TCTCTGAGACTGAGCTGTGCCGCCTCCGGCTTCACCTTTTCCTCTTACGG |
| | | | CATGCACTGGGTGCGGCAGGCCCCCGGCAAGGGACTGGAGTGGGTGGC |
| | | | CTTCATCAGATATGATGGCAGCAATAAGTACTATGCCGACTCCGTGAAG |
| | | | GGCAGGTTTACCATCAGCCGGGACAACAGCAAGAATACACTGTATCTG |
| | | | CAGATGAACAGCCTGAGAGCCGAGGATACCGCCGTGTACTATTGCAAG |
| | | | ACACACGGCTCCCACGACAATTGGGGCCAGGGCACCATGGTGACAGTG |
| | | | AGCTCC |
| 128 | 21421 | Full nt | GAGCCAAAGAGCTCCGACAAGACCCACACATGCCCACCTTGTCCGGCG |
| | | | CCAGAGGCCGCCGGAGGACCTAGCGTGTTCCTGTTTCCACCCAAGCCAA |
| | | | AGGATACCCTGATGATCAGCAGGACCCCAGAGGTGACATGCGTGGTGG |
| | | | TGTCTGTGAGCCACGAGGACCCCGAGGTGAAGTTTAACTGGTACGTGG |
| | | | ATGGCGTGGAGGTGCACAATGCCAAGACAAAGCCACGGGAGGAGCAGT |
| | | | ACAACTCCACCTATAGAGTGGTGTCTGTGCTGACAGTGCTGCACCAGGA |
| | | | CTGGCTGAACGGCAAGGAGTATAAGTGCAAGGTGAGCAATAAGGCCCT |
| | | | GCCTGCCCCAATCGAGAAGACCATCTCCAAGGCCAAGGGCCAGCCTCG |
| | | | CGAACCTCAGGTGTACGTGTATCCTCCATCCCGCGACGAGCTGACCAAG |
| | | | AACCAGGTGTCTCTGACATGTCTGGTGAAGGGCTTCTACCCCTCTGATA |
| | | | TCGCCGTGGAGTGGGAGAGCAATGGCCAGCCTGAGAACAATTATAAGA |
| | | | CCACACCCCCTGTGCTGGACAGCGATGGCTCCTTCGCCCTGGTGAGCAA |
| | | | GCTGACCGTGGATAAGTCCAGATGGCAGCAGGGCAACGTGTTTTCCTGT |
| | | | TCTGTGATGCACGAGGCCCTGCACAATCACTACACACAGAAGAGCCTG |
| | | | AGCTTAAGCCCAGGAGGAGGAGGAGGCTCTGGAGGAGGAGGCAGCAT |
| | | | CTGGGAGCTGAAGAAGGACGTGTATGTGGTGGAGCTGGATTGGTACCC |
| | | | TGATGCCCCAGGCGAGATGGTGGTGCTGACCTGCGACACACCCGAGGA |
| | | | GGATGGCATCACCTGGACACTGGACCAGAGCTCCGAGGTGCTGGGCAG |
| | | | CGGCAAGACCCTGACAATCCAGGTGAAGGAGTTCGGCGATGCCGGACA |
| | | | GTACACCTGTCACAAGGGAGGAGAGGTGCTGAGCCACTCCCTGCTGCT |
| | | | GCTGCACAAGAAGGAGGATGGCATCTGGTCCACAGACATCCTGAAGGA |
| | | | TCAGAAGGAGCCAAAGAACAAGACCTTCCTGCGGTGCGAGGCCAAGAA |
| | | | TTATAGCGGCAGATTCACCTGTTGGTGGCTGACCACAATCTCCACCGAC |
| | | | CTGACATTTTCTGTGAAGTCTAGCAGGGGCTCCTCTGATCCCCAGGGAG |
| | | | TGACATGCGGAGCCGCCACCCTGTCCGCCGAGCGGGTGAGAGGCGACA |
| | | | ACAAGGAGTACGAGTATTCTGTGGAGTGCCAGGAGGATAGCGCCTGTC |
| | | | CCGCCGCCGAGGAGAGCCTGCCTATCGAAGTGATGGTGGACGCCGTGC |
| | | | ACAAGCTGAAGTACGAGAATTATACAAGCTCCTTCTTTATCCGGGACAT |
| | | | CATCAAGCCAGATCCCCCTAAGAACCTGCAGCTGAAGCCCCTGAAGAA |
| | | | TAGCAGACAGGTGGAGGTGTCCTGGGAGTACCCTGATACCTGGAGCAC |
| | | | ACCACACTCCTATTTCTCTCTGACCTTTTGCGTGCAGGTGCAGGGCAAG |
| | | | TCTAAGAGGGAGAAGAAGGACCGCGTGTTTACCGATAAGACAAGCGCC |
| | | | ACCGTGATCTGTAGGAAGAACGCCTCTATCAGCGTGCGGGCACAGGAC |
| | | | CGGTACTATTCTAGCTCCTGGAGCGAGTGGGCCTCCGTGCCTTGCTCTG |
| | | | GAGGAGGAGGCAGCGGCGGAGGAGGCTCCATGAGCGGGCGGAGCGCC |
| | | | AACGCAGGGGGCGGCGGCTCTGGCGGCGGCGGCAGCGGAGGCGGCGG |
| | | | CAGCCAGTCCGTGCTGACACAGCCACCATCTGTGAGCGGAGCCCCCGG |
| | | | ACAGAGGGTGACCATCTCCTGTTCTGGCAGCCGCTCCAACATCGGCTCC |
| | | | AATACAGTGAAGTGGTATCAGCAGCTGCCAGGAACCGCCCCTAAGCTG |
| | | | CTGATCTACTATAACGACCAGAGGCCAAGCGGAGTGCCAGATCGCTTCT |
| | | | CTGGCAGCAAGTCCGGCACATCTGCCAGCCTGGCCATCACCGGACTGCA |
| | | | GGCAGAGGACGAGGCCGATTACTATTGCCAGTCCTACGATCGGTATAC |
| | | | ACACCCTGCCCTGCTGTTTGGCACCGGCACAAAGGTGACCGTGCTGGGC |
| | | | GGAGGAGGCTCCGGAGGAGGAGGCTCTGGAGGAGGCGGCAGCCAGGT |
| | | | GCAGCTGGTGGAGAGCGGCGGCGGCGTGGTGCAGCCCGGCAGGAGCCT |
| | | | GCGCCTGTCCTGTGCAGCCTCTGGCTTCACCTTTTCTAGCTACGGCATGC |
| | | | ACTGGGTGCGGCAGGCCCCTGGCAAGGGACTGGAGTGGGTGGCCTTCA |
| | | | TCAGATATGACGGCTCCAATAAGTACTATGCCGATTCTGTGAAGGGCCG |
| | | | GTTTACAATCAGCAGAGATAACTCCAAGAATACCCTGTACCTGCAGATG |
| | | | AACTCCCTGAGAGCCGAGGACACAGCCGTGTACTATTGTAAGACCCAC |
| | | | GGCTCTCACGATAATTGGGGCCAGGGCACAATGGTGACCGTGTCCTCT |
| 129 | 21423 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCAGGAGGCTCCGCCGATGGAGGAATCTGGGAGCTGAAGA |
| | | | AGGACGTGTACGTGGTGGAGCTGGACTGGTACCCTGATGCCCCTGGCG |
| | | | AGATGGTGGTGCTGACCTGCGACACACCCGAGGAGGATGGCATCACCT |
| | | | GGACACTGGATCAGAGCTCCGAGGTGCTGGGCAGCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGTCTACAGACATCCTGAAGGATCAGAAGGAGCCTA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCCGGGGCTCCTCTGACCCACAGGGAGTGACATGCGGAGCC |
| | | | GCCACCCTGTCCGCCGAGCGGGTGAGAGGCGATAACAAGGAGTACGAG |
| | | | TATTCTGTGGAGTGCCAGGAGGACAGCGCCTGTCCAGCCGCCGAGGAG |
| | | | AGCCTGCCAATCGAAGTGATGGTGGATGCCGTGCACAAGCTGAAGTAC |
| | | | GAGAATTATACAAGCTCCTTCTTTATCCGGGACATCATCAAGCCCGATC |
| | | | CCCCTAAGAACCTGCAGCTGAAGCCTCTGAAGAATAGCAGACAGGTGG |
| | | | AGGTGTCCTGGGAGTACCCCGACACCTGGAGCACACCTCACTCTTATTT |
| | | | CAGCCTGACCTTTTGCGTGCAGGTGCAGGGCAAGTCTAAGCGGGAGAA |
| | | | GAAGGACAGAGTGTTCACCGATAAGACAAGCGCCACCGTGATCTGTCG |
| | | | GAAGAACGCCAGCATCTCCGTGAGGGCACAGGACCGGTACTATTCTAG |
| | | | CTCCTGGTCCGAGTGGGCCTCTGTGCCCTGTAGCGGAGGAGGAGGCAG |
| | | | CGGAGGAGGAGGCTCCGGAGGCGGCGGCTCTAGAAATCTGCCAGTGGC |
| | | | CACCCCTGACCCAGGCATGTTCCCCTGCCTGCACCACAGCCAGAACCTG |
| | | | CTGAGGGCCGTGTCCAATATGCTGCAGAAGGCCCGCCAGACACTGGAG |
| | | | TTTTACCCTTGTACCTCCGAGGAGATCGACCACGAGGATATCACAAAGG |
| | | | ATAAGACCTCTACAGTGGAGGCCTGCCTGCCACTGGAGCTGACCAAGA |
| | | | ACGAGTCCTGTCTGAACAGCCGGGAGACCAGCTTCATCACCAACGGCTC |
| | | | CTGCCTGGCCTCTAGAAAGACAAGCTTTATGATGGCCCTGTGCCTGAGC |
| | | | AGCATCTACGAGGACCTGAAGATGTATCAGGTGGAGTTCAAGACCATG |
| | | | AACGCCAAGCTGCTGATGGACCCCAAGCGGCAGATCTTTCTGGATCAG |
| | | | AATATGCTGGCCGTGATCGACGAGCTGATGCAGGCCCTGAACTTCAATT |
| | | | CCGAGACAGTGCCTCAGAAGTCCTCTCTGGAGGAGCCAGATTTCTACAA |
| | | | GACCAAGATCAAGCTGTGCATCCTGCTGCACGCCTTCCGGATCAGAGCC |
| | | | GTGACCATCGACAGAGTGATGAGCTATCTGAACGCCTCCGGAGGAGGA |
| | | | GGCTCTGGAGGAGGCGGCAGCGGCGGCGGCGGCTCTATGAGCGGGCGG |
| | | | AGCGCCAACGCAGGGGGAGGAGGCTCCGGCGGGGGAGGCTCTGGCGG |
| | | | CGGAGGCAGCGGAGGAGGCGGCTCCCAGTCTGTGCTGACACAGCCACC |
| | | | AAGCGTGTCCGGAGCCCCCGGACAGAGGGTGACCATCTCTTGTAGCGG |
| | | | CTCCAGATCTAACATCGGCTCCAATACAGTGAAGTGGTATCAGCAGCTG |
| | | | CCAGGAACCGCCCCCAAGCTGCTGATCTACTATAACGATCAGCGGCCTA |
| | | | GCGGCGTGCCAGACAGATTCAGCGGCTCCAAGTCTGGCACAAGCGCCT |
| | | | CCCTGGCCATCACCGGACTGCAGGCCGAGGACGAGGCCGATTACTATT |
| | | | GCCAGTCCTACGATAGGTATACACACCCTGCCCTGCTGTTTGGCACCGG |
| | | | CACAAAGGTGACCGTGCTGGGCGGAGGAGGCTCCGGCGGAGGCGGCTC |
| | | | TGGCGGCGGCGGCAGCCAGGTGCAGCTGGTGGAGAGCGGCGGCGGCGT |
| | | | GGTGCAGCCCGGCAGAAGCCTGCGGCTGAGCTGTGCAGCCAGCGGCTT |
| | | | CACCTTTAGCTCCTACGGCATGCACTGGGTGAGGCAGGCCCCTGGCAAG |
| | | | GGACTGGAGTGGGTGGCCTTCATCCGCTATGATGGCTCCAATAAGTACT |
| | | | ATGCCGACTCTGTGAAGGGCAGGTTTACAATCTCCCGCGACAACTCTAA |
| | | | GAATACCCTGTACCTGCAGATGAACAGCCTGCGCGCCGAGGATACAGC |
| | | | CGTGTACTATTGCAAGACCCACGGCTCCCACGACAATTGGGGCCAGGG |
| | | | CACAATGGTGACCGTGTCTAGC |
| 130 | 21446 | Full nt | CAGGTGCAGCTGGTGGAGAGCGGCGGCGGCGTGGTGCAGCCCGGCAGG |
| | | | AGCCTGCGCCTGTCCTGCGCAGCCTCTGGCTTCACCTTTAGCTCCTACGG |
| | | | CATGCACTGGGTGCGGCAGGCCCCAGGCAAGGGACTGGAGTGGGTGGC |
| | | | CTTCATCAGATATGACGGCTCCAACAAGTACTATGCCGATTCTGTGAAG |
| | | | GGCAGGTTTACAATCAGCCGCGACAACTCCAAGAATACCCTGTACCTGC |
| | | | AGATGAACAGCCTGAGGGCCGAGGACACAGCCGTGTACTATTGCAAGA |
| | | | CCCACGGCTCCCACGATAATTGGGGCCAGGGCACCATGGTGACAGTGT |
| | | | CTAGTGGAGGAGGAGGCTCTGGAGGAGGAGGCAGCGGAGGAGGAGGC |
| | | | AGCCAGTCCGTGCTGACACAGCCCCCTTCTGTGAGCGGAGCCCCCGGAC |
| | | | AGAGGGTGACCATCTCCTGTTCTGGCAGCAGATCCAACATCGGCAGCA |
| | | | ATACAGTGAAGTGGTATCAGCAGCTGCCAGGAACCGCCCCTAAGCTGC |
| | | | TGATCTACTATAACGACCAGAGGCCATCCGGAGTGCCAGATCGCTTCTC |
| | | | TGGCAGCAAGTCCGGCACATCTGCCAGCCTGGCCATCACCGGACTGCA |
| | | | GGCAGAGGACGAGGCCGATTACTATTGTCAGAGCTACGACAGGTATAC |
| | | | ACACCCCGCCCTGCTGTTTGGCACCGGCACAAAGGTGACCGTGCTGGGC |
| | | | GGCGGCGGCTCTGGCGGCGGCGGCTCCATGAGCGGGCGGAGCGCCAAC |
| | | | GCAGGGGGAGGAGGCTCCGGAGGAGGAGGCTCTATCTGGGAGCTGAAG |
| | | | AAGGACGTGTACGTGGTGGAGCTGGACTGGTACCCTGATGCCCCAGGC |
| | | | GAGATGGTGGTGCTGACCTGCGACACACCTGAGGAGGATGGCATCACC |
| | | | TGGACACTGGACCAGTCCTCTGAGGTGCTGGGCTCCGGCAAGACCCTGA |
| | | | CAATCCAGGTGAAGGAGTTCGGCGATGCCGGACAGTACACCTGTCACA |
| | | | AGGGAGGAGAGGTGCTGAGCCACTCCCTGCTGCTGCTGCACAAGAAGG |
| | | | AGGACGGCATCTGGTCTACAGACATCCTGAAGGATCAGAAGGAGCCTA |
| | | | AGAACAAGACCTTCCTGAGGTGCGAGGCCAAGAATTATAGCGGCAGAT |
| | | | TCACCTGTTGGTGGCTGACCACAATCAGCACCGACCTGACATTTTCCGT |
| | | | GAAGAGCTCCCGGGGCTCTAGCGATCCACAGGGAGTGACATGCGGAGC |
| | | | CGCCACCCTGAGCGCCGAGCGGGTGAGAGGCGACAACAAGGAGTACGA |
| | | | GTATAGCGTGGAGTGCCAGGAGGATTCCGCCTGTCCCGCCGCCGAGGA |
| | | | GTCCCTGCCTATCGAAGTGATGGTGGACGCCGTGCACAAGCTGAAGTAC |
| | | | GAGAATTATACCTCCTCTTTCTTTATCCGGGACATCATCAAGCCAGATC |
| | | | CACCCAAGAACCTGCAGCTGAAGCCCCTGAAGAACAGCAGACAGGTGG |
| | | | AGGTGAGCTGGGAGTACCCTGATACCTGGAGCACACCACACTCCTATTT |
| | | | CTCTCTGACCTTTTGCGTGCAGGTGCAGGGCAAGTCCAAGCGGGAGAA |
| | | | GAAGGACAGAGTGTTTACCGATAAGACATCTGCCACCGTGATCTGTAG |
| | | | AAAGAACGCCAGCATCAGCGTGCGGGCACAGGACCGGTACTATAGCTC |
| | | | CTCTTGGTCTGAGTGGGCCAGCGTGCCTTGTTCC |
| 131 | 21447 | Full nt | CAGGTGCAGCTGGTGGAGAGCGGAGGAGGAGTGGTGCAGCCAGGCAG |
| | | | GTCTCTGCGCCTGAGCTGCGCAGCCTCCGGCTTCACCTTTAGCTCCTACG |
| | | | GCATGCACTGGGTGCGGCAGGCCCCTGGCAAGGGACTGGAGTGGGTGG |
| | | | CCTTCATCAGATATGACGGCTCTAACAAGTACTATGCCGATAGCGTGAA |
| | | | GGGCCGGTTTACCATCTCTAGAGATAACAGCAAGAATACACTGTACCTG |
| | | | CAGATGAACTCTCTGAGGGCCGAGGATACCGCCGTGTACTATTGCAAG |
| | | | ACACACGGCTCCCACGACAATTGGGGCCAGGGCACCATGGTGACAGTG |
| | | | TCTAGCGGAGGAGGAGGCTCCGGAGGAGGAGGCTCTGGCGGCGGCGGC |
| | | | TCTCAGAGCGTGCTGACCCAGCCACCTTCCGTGTCTGGAGCCCCCGGAC |
| | | | AGAGGGTGACAATCAGCTGTTCCGGCTCTAGAAGCAACATCGGCAGCA |
| | | | ATACCGTGAAGTGGTATCAGCAGCTGCCTGGCACAGCCCCAAAGCTGCT |
| | | | GATCTACTATAATGACCAGAGGCCCTCCGGCGTGCCTGATCGCTTCTCC |
| | | | GGCTCTAAGAGCGGCACCTCCGCCTCTCTGGCCATCACAGGCCTGCAGG |
| | | | CAGAGGACGAGGCAGATTACTATTGTCAGAGCTACGACAGATACACCC |
| | | | ACCCCGCCCTGCTGTTTGGCACCGGCACAAAGGTGACAGTGCTGGGCG |
| | | | GAGGAGGCAGCGGCGGCGGAGGCTCCGGAGGCGGCGGCTCTGGAGGA |
| | | | GGCGGCAGCATGAGCGGGCGGAGCGCCAACGCAGGGGGCGGCGGCAG |
| | | | CGGCGGCGGCGGCTCCGGAGGAGGGGGCTCTCGCAATCTGCCTGTGGC |
| | | | CACCCCAGATCCCGGCATGTTCCCATGCCTGCACCACTCCCAGAACCTG |
| | | | CTGAGGGCCGTGTCTAATATGCTGCAGAAGGCCCGCCAGACCCTGGAG |
| | | | TTTTACCCCTGTACAAGCGAGGAGATCGACCACGAGGATATCACCAAG |
| | | | GACAAGACCTCCACAGTGGAGGCCTGCCTGCCTCTGGAGCTGACAAAG |
| | | | AACGAGTCCTGTCTGAACAGCCGGGAGACCAGCTTCATCACAAACGGC |
| | | | TCCTGCCTGGCCTCTCGCAAGACCAGCTTTATGATGGCCCTGTGCCTGA |
| | | | GCTCTATCTACGAGGATCTGAAGATGTATCAGGTGGAGTTCAAGACAAT |
| | | | GAACGCCAAGCTGCTGATGGACCCCAAGCGGCAGATCTTTCTGGATCA |
| | | | GAATATGCTGGCCGTGATCGACGAGCTGATGCAGGCCCTGAACTTTAAT |
| | | | AGCGAGACCGTGCCACAGAAGAGCTCCCTGGAGGAGCCCGATTTCTAC |
| | | | AAGACAAAGATCAAGCTGTGCATCCTGCTGCACGCCTTTCGGATCAGAG |
| | | | CCGTGACCATCGACAGAGTGATGAGCTACCTGAACGCCAGC |
| 132 | 21451 | Full nt | CAGGTGCAGCTGGTGGAGAGCGGCGGCGGCGTGGTGCAGCCTGGCCGG |
| | | | AGCCTGAGACTGTCCTGCGCAGCCTCTGGCTTCACCTTTAGCTCCTACG |
| | | | GCATGCACTGGGTGCGGCAGGCCCCTGGCAAGGGACTGGAGTGGGTGG |
| | | | CCTTCATCAGATATGACGGCTCCAACAAGTACTATGCCGATTCTGTGAA |
| | | | GGGCAGGTTTACCATCAGCCGCGACAACTCCAAGAATACACTGTACCTG |
| | | | CAGATGAACAGCCTGCGGGCAGAGGACACCGCCGTGTACTATTGCAAG |
| | | | ACACACGGCTCCCACGATAATTGGGGCCAGGGCACCATGGTGACAGTG |
| | | | TCTTCCGGAGGAGGAGGCTCTGGAGGAGGAGGCAGCGGAGGAGGAGG |
| | | | CAGCCAGTCCGTGCTGACCCAGCCACCTTCTGTGAGCGGAGCCCCCGGA |
| | | | CAGAGGGTGACAATCTCCTGTTCTGGCAGCCGCTCCAACATCGGCTCTA |
| | | | ATACCGTGAAGTGGTATCAGCAGCTGCCTGGCACAGCCCCAAAGCTGCT |
| | | | GATCTACTATAACGACCAGAGACCCTCCGGCGTGCCTGATAGATTCTCT |
| | | | GGCAGCAAGTCCGGCACCTCTGCCAGCCTGGCCATCACAGGCCTGCAG |
| | | | GCAGAGGACGAGGCAGATTACTATTGTCAGAGCTACGACAGATACACC |
| | | | CACCCAGCCCTGCTGTTTGGCACCGGCACAAAGGTGACAGTGCTGGGC |
| | | | GGAGGAGGCTCCGGCGGCGGAGGCTCTGGCGGCGGCGGCAGCGGAGG |
| | | | CGGCGGCTCCATGAGCGGGCGGAGCGCCAACGCAGGGGGCGGCGGCTC |
| | | | CGGCGGCGGCGGCTCTGGAGGAGGCGGCAGCGAGCCCAAGTCCTCTGA |
| | | | TAAGACCCACACATGCCCACCCTGTCCGGCGCCAGAGGCAGCAGGAGG |
| | | | ACCAAGCGTGTTCCTGTTTCCACCCAAGCCCAAAGACACCCTGATGATT |
| | | | AGCCGAACCCCTGAAGTCACATGCGTGGTCGTGTCCGTGTCTCACGAGG |
| | | | ACCCAGAAGTCAAGTTCAACTGGTACGTGGATGGCGTCGAGGTGCATA |
| | | | ATGCCAAGACAAAACCCCGGGAGGAACAGTACAACAGCACCTATAGAG |
| | | | TCGTGTCCGTCCTGACAGTGCTGCACCAGGATTGGCTGAACGGCAAGGA |
| | | | ATATAAGTGCAAAGTGTCCAATAAGGCCCTGCCCGCTCCTATCGAGAAA |
| | | | ACCATTTCTAAGGCAAAAGGCCAGCCTCGCGAACCACAGGTCTACGTG |
| | | | CTGCCTCCATCCCGGGACGAGCTGACAAAGAACCAGGTCTCTCTGCTGT |
| | | | GCCTGGTGAAAGGCTTCTATCCATCAGATATTGCTGTGGAGTGGGAAAG |
| | | | CAATGGGCAGCCCGAGAACAATTACCTGACTTGGCCCCCTGTGCTGGAC |
| | | | TCTGATGGGAGTTTCTTTCTGTATTCTAAGCTGACCGTGGATAAAAGTA |
| | | | GGTGGCAGCAGGGAAATGTCTTTAGTTGTTCAGTGATGCATGAAGCCCT |
| | | | GCATAACCACTACACCCAGAAAAGCCTGTCCCTGTCCCCCGGA |
| 133 | 21452 | Full nt | CAGGTGCAGCTGGTGGAGAGCGGAGGAGGAGTGGTGCAGCCTGGCAGG |
| | | | TCCCTGCGCCTGTCTTGCGCAGCCAGCGGCTTCACCTTTAGCTCCTACGG |
| | | | CATGCACTGGGTGCGGCAGGCCCCCGGCAAGGGACTGGAGTGGGTGGC |
| | | | CTTCATCAGATATGACGGCAGCAACAAGTACTATGCCGATTCCGTGAAG |
| | | | GGCCGGTTTACCATCTCCAGAGATAACTCTAAGAATACACTGTACCTGC |
| | | | AGATGAACAGCCTGAGGGCCGAGGATACCGCCGTGTACTATTGCAAGA |
| | | | CACACGGCTCCCACGACAATTGGGGCCAGGGCACCATGGTGACAGTGT |
| | | | CTTCCGGAGGAGGAGGCAGCGGAGGAGGAGGCTCCGGAGGCGGCGGC |
| | | | TCCCAGTCTGTGCTGACCCAGCCACCTAGCGTGTCCGGAGCCCCCGGCC |
| | | | AGCGGGTGACAATCTCTTGTAGCGGCTCCAGATCTAACATCGGCAGCAA |
| | | | TACCGTGAAGTGGTATCAGCAGCTGCCCGGCACAGCCCCTAAGCTGCTG |
| | | | ATCTACTATAATGACCAGAGGCCATCCGGCGTGCCCGATCGCTTCAGCG |
| | | | GCTCCAAGTCTGGCACCAGCGCCTCCCTGGCCATCACAGGCCTGCAGGC |
| | | | AGAGGACGAGGCAGATTACTATTGTCAGAGCTACGACAGATACACCCA |
| | | | CCCTGCCCTGCTGTTTGGCACCGGCACAAAGGTGACAGTGCTGGGCGGA |
| | | | GGAGGCTCTGGAGGAGGAGGCAGCGGCGGCGGCGGCTCTATGAGCGGG |
| | | | CGGAGCGCCAACGCAGGGGGAGGAGGCTCCGGAGGAGGAGGCTCTGG |
| | | | CGGCGGCGGCAGCGGAGGAGGGGGCTCCCGCAATCTGCCCGTGGCCAC |
| | | | CCCTGATCCAGGCATGTTCCCTTGCCTGCACCACTCTCAGAACCTGCTG |
| | | | AGGGCCGTGAGCAATATGCTGCAGAAGGCCCGCCAGACCCTGGAGTTT |
| | | | TACCCATGTACATCCGAGGAGATCGACCACGAGGATATCACCAAGGAC |
| | | | AAGACCTCTACAGTGGAGGCCTGCCTGCCCCTGGAGCTGACAAAGAAC |
| | | | GAGAGCTGTCTGAACAGCCGGGAGACCAGCTTCATCACAAACGGCAGC |
| | | | TGCCTGGCCTCCCGCAAGACCTCTTTTATGATGGCCCTGTGCCTGAGCTC |
| | | | TATCTACGAGGATCTGAAGATGTATCAGGTGGAGTTCAAGACAATGAA |
| | | | CGCCAAGCTGCTGATGGACCCTAAGCGGCAGATCTTTCTGGATCAGAAT |
| | | | ATGCTGGCCGTGATCGACGAGCTGATGCAGGCCCTGAACTTTAATAGCG |
| | | | AGACCGTGCCTCAGAAGAGCTCCCTGGAGGAGCCAGATTTCTACAAGA |
| | | | CAAAGATCAAGCTGTGCATCCTGCTGCACGCCTTTCGGATCAGAGCCGT |
| | | | GACCATCGACAGAGTGATGTCTTACCTGAACGCCAGCGGCGGCGGAGG |
| | | | CTCCGGAGGAGGCGGCTCTGAGCCAAAGTCTAGCGACAAGACCCACAC |
| | | | ATGCCCACCCTGTCCGGCGCCAGAGGCTGCAGGAGGACCAAGCGTGTT |
| | | | CCTGTTTCCACCCAAGCCTAAAGACACACTGATGATTTCCCGAACCCCC |
| | | | GAAGTCACATGCGTGGTCGTGTCTGTGAGTCACGAGGACCCTGAAGTCA |
| | | | AGTTCAACTGGTACGTGGATGGCGTCGAGGTGCATAATGCCAAGACTA |
| | | | AACCTAGGGAGGAACAGTACAACTCAACCTATCGCGTCGTGAGCGTCC |
| | | | TGACAGTGCTGCACCAGGATTGGCTGAACGGCAAAGAATATAAGTGCA |
| | | | AAGTGAGCAATAAGGCCCTGCCCGCTCCTATCGAGAAAACCATTTCCAA |
| | | | GGCTAAAGGGCAGCCTCGCGAACCACAGGTCTACGTGTATCCTCCAAG |
| | | | CCGGGACGAGCTGACAAAGAACCAGGTCTCCCTGACTTGTCTGGTGAA |
| | | | AGGGTTTTACCCTAGTGATATCGCTGTGGAGTGGGAATCAAATGGACAG |
| | | | CCAGAGAACAATTATAAGACTACCCCCCCTGTGCTGGACAGTGATGGGT |
| | | | CATTCGCACTGGTCTCCAAGCTGACAGTGGACAAATCTCGGTGGCAGCA |
| | | | GGGAAATGTCTTTTCATGTAGCGTGATGCATGAAGCACTGCACAACCAT |
| | | | TACACCCAGAAGTCACTGTCACTGTCACCAGGA |
| 134 | 22203 | Full nt | CAGGTGCAGCTGGTGGAGAGCGGCGGCGGCGTGGTGCAGCCTGGCCGG |
| | | | TCCCTGAGACTGTCTTGCGCAGCCAGCGGCTTCACCTTTAGCTCCGCTG |
| | | | GCATGCACTGGGTGCGGCAGGCCCCTGGCAAGGGACTGGAGTGGGTGG |
| | | | CCTTCATCAGATATGACGGCTCTAACAAGTACTATGCCGATAGCGTGAA |
| | | | GGGCAGGTTTACCATCTCCCGCGACAACTCTAAGAATACACTGTACCTG |
| | | | CAGATGAACTCCCTGCGGGCAGAGGACACCGCCGTGTACTATTGCAAG |
| | | | ACACACGGCTCTCACGATAATTGGGGCCAGGGCACCATGGTGACAGTG |
| | | | TCTAGCGGAGGAGGAGGCAGCGGAGGAGGAGGCTCCGGCGGCGGCGG |
| | | | CAGCCAGTCCGTGCTGACCCAGCCACCTTCTGTGAGCGGAGCCCCCGGA |
| | | | CAGAGGGTGACAATCTCCTGTTCTGGCAGCCGCTCCAACATCGGCAGCA |
| | | | ATACCGTGAAGTGGTATCAGCAGCTGCCTGGCACAGCCCCAAAGCTGCT |
| | | | GATCTACTATAATGACCAGAGACCCTCCGGCGTGCCTGATAGATTCTCT |
| | | | GGCAGCAAGTCCGGCACCTCTGCCAGCCTGGCCATCACAGGACTGCAG |
| | | | GCAGAGGACGAGGCAGATTACTATTGTCAGTCCTACGACAGATATACC |
| | | | CACCCAGCCCTGCTGTTTGGCACCGGCACAAAGGTGACAGTGCTGGCCG |
| | | | CCGAGCCCAAGTCCTCTGATAAGACCCACACATGCCCACCCTGTCCGGC |
| | | | GCCAGAGGCAGCAGGAGGACCAAGCGTGTTCCTGTTTCCACCCAAGCC |
| | | | CAAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAACCACAGGTCTACGTGCTGCCTCCATCCCGGGACGAGCTGACAAAG |
| | | | AACCAGGTCTCTCTGCTGTGCCTGGTGAAAGGCTTCTATCCATCAGATA |
| | | | TTGCTGTGGAGTGGGAAAGCAATGGGCAGCCCGAGAACAATTACCTGA |
| | | | CTTGGCCCCCTGTGCTGGACTCTGATGGGAGTTTCTTTCTGTATTCTAAG |
| | | | CTGACCGTGGATAAAAGTAGGTGGCAGCAGGGAAATGTCTTTAGTTGTT |
| | | | CAGTGATGCATGAAGCCCTGCATAACCACTACACCCAGAAAAGCCTGT |
| | | | CCCTGTCCCCCGGA |
| 135 | 22206 | Full nt | CAGGTGCAGCTGGTGGAGAGCGGCGGCGGCGTGGTGCAGCCTGGCCGG |
| | | | TCCCTGAGACTGTCTTGCGCAGCCAGCGGCGTGACCTTTAGCTCCTACG |
| | | | GCATGCACTGGGTGCGGCAGGCCCCTGGCAAGGGACTGGAGTGGGTGG |
| | | | CCTTCATCAGATATGACGGCTCTAACAAGTACTATGCCGATAGCGTGAA |
| | | | GGGCAGGTTTACCATCTCCCGCGACAACTCTAAGAATACACTGTACCTG |
| | | | CAGATGAACTCCCTGCGGGCAGAGGACACCGCCGTGTACTATTGCAAG |
| | | | ACACACGGCTCTCACGATAATTGGGGCCAGGGCACCATGGTGACAGTG |
| | | | TCTAGCGGAGGAGGAGGCAGCGGAGGAGGAGGCTCCGGCGGCGGCGG |
| | | | CAGCCAGTCCGTGCTGACCCAGCCACCTTCTGTGAGCGGAGCCCCCGGA |
| | | | CAGAGGGTGACAATCTCCTGTTCTGGCAGCCGCTCCAACATCGGCAGCA |
| | | | ATACCGTGAAGTGGTATCAGCAGCTGCCTGGCACAGCCCCAAAGCTGCT |
| | | | GATCTACTATAATGACCAGAGACCCTCCGGCGTGCCTGATAGATTCTCT |
| | | | GGCAGCAAGTCCGGCACCTCTGCCAGCCTGGCCATCACAGGACTGCAG |
| | | | GCAGAGGACGAGGCAGATTACTATTGTCAGTCCTACGACAGATATACC |
| | | | CACCCAGCCCTGCTGTTTGGCACCGGCACAAAGGTGACAGTGCTGGCCG |
| | | | CCGAGCCCAAGTCCTCTGATAAGACCCACACATGCCCACCCTGTCCGGC |
| | | | GCCAGAGGCAGCAGGAGGACCAAGCGTGTTCCTGTTTCCACCCAAGCC |
| | | | CAAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAACCACAGGTCTACGTGCTGCCTCCATCCCGGGACGAGCTGACAAAG |
| | | | AACCAGGTCTCTCTGCTGTGCCTGGTGAAAGGCTTCTATCCATCAGATA |
| | | | TTGCTGTGGAGTGGGAAAGCAATGGGCAGCCCGAGAACAATTACCTGA |
| | | | CTTGGCCCCCTGTGCTGGACTCTGATGGGAGTTTCTTTCTGTATTCTAAG |
| | | | CTGACCGTGGATAAAAGTAGGTGGCAGCAGGGAAATGTCTTTAGTTGTT |
| | | | CAGTGATGCATGAAGCCCTGCATAACCACTACACCCAGAAAAGCCTGT |
| | | | CCCTGTCCCCCGGA |
| 136 | 22207 | Full nt | CAGGTGCAGCTGGTGGAGAGCGGCGGCGGCGTGGTGCAGCCTGGCCGG |
| | | | TCCCTGAGACTGTCTTGCGCAGCCAGCGGCTTCACCTTTAGCTCCTACG |
| | | | GCATGCACTGGGTGCGGCAGGCCCCTGGCAAGGGACTGGAGTGGGTGG |
| | | | CCTTCATCAGAGTGGACGGCTCTAACAAGTACTATGCCGATAGCGTGAA |
| | | | GGGCAGGTTTACCATCTCCCGCGACAACTCTAAGAATACACTGTACCTG |
| | | | CAGATGAACTCCCTGCGGGCAGAGGACACCGCCGTGTACTATTGCAAG |
| | | | ACACACGGCTCTCACGATAATTGGGGCCAGGGCACCATGGTGACAGTG |
| | | | TCTAGCGGAGGAGGAGGCAGCGGAGGAGGAGGCTCCGGCGGCGGCGG |
| | | | CAGCCAGTCCGTGCTGACCCAGCCACCTTCTGTGAGCGGAGCCCCCGGA |
| | | | CAGAGGGTGACAATCTCCTGTTCTGGCAGCCGCTCCAACATCGGCAGCA |
| | | | ATACCGTGAAGTGGTATCAGCAGCTGCCTGGCACAGCCCCAAAGCTGCT |
| | | | GATCTACTATAATGACCAGAGACCCTCCGGCGTGCCTGATAGATTCTCT |
| | | | GGCAGCAAGTCCGGCACCTCTGCCAGCCTGGCCATCACAGGACTGCAG |
| | | | GCAGAGGACGAGGCAGATTACTATTGTCAGTCCTACGACAGATATACC |
| | | | CACCCAGCCCTGCTGTTTGGCACCGGCACAAAGGTGACAGTGCTGGCCG |
| | | | CCGAGCCCAAGTCCTCTGATAAGACCCACACATGCCCACCCTGTCCGGC |
| | | | GCCAGAGGCAGCAGGAGGACCAAGCGTGTTCCTGTTTCCACCCAAGCC |
| | | | CAAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAACCACAGGTCTACGTGCTGCCTCCATCCCGGGACGAGCTGACAAAG |
| | | | AACCAGGTCTCTCTGCTGTGCCTGGTGAAAGGCTTCTATCCATCAGATA |
| | | | TTGCTGTGGAGTGGGAAAGCAATGGGCAGCCCGAGAACAATTACCTGA |
| | | | CTTGGCCCCCTGTGCTGGACTCTGATGGGAGTTTCTTTCTGTATTCTAAG |
| | | | CTGACCGTGGATAAAAGTAGGTGGCAGCAGGGAAATGTCTTTAGTTGTT |
| | | | CAGTGATGCATGAAGCCCTGCATAACCACTACACCCAGAAAAGCCTGT |
| | | | CCCTGTCCCCCGGA |
| 137 | 22208 | Full nt | CAGGTGCAGCTGGTGGAGAGCGGCGGCGGCGTGGTGCAGCCTGGCCGG |
| | | | TCCCTGAGACTGTCTTGCGCAGCCAGCGGCTTCACCTTTAGCTCCTACG |
| | | | GCATGCACTGGGTGCGGCAGGCCCCTGGCAAGGGACTGGAGTGGGTGG |
| | | | CCTTCATCGAGTATGACGGCTCTAACAAGTACTATGCCGATAGCGTGAA |
| | | | GGGCAGGTTTACCATCTCCCGCGACAACTCTAAGAATACACTGTACCTG |
| | | | CAGATGAACTCCCTGCGGGCAGAGGACACCGCCGTGTACTATTGCAAG |
| | | | ACACACGGCTCTCACGATAATTGGGGCCAGGGCACCATGGTGACAGTG |
| | | | TCTAGCGGAGGAGGAGGCAGCGGAGGAGGAGGCTCCGGCGGCGGCGG |
| | | | CAGCCAGTCCGTGCTGACCCAGCCACCTTCTGTGAGCGGAGCCCCCGGA |
| | | | CAGAGGGTGACAATCTCCTGTTCTGGCAGCCGCTCCAACATCGGCAGCA |
| | | | ATACCGTGAAGTGGTATCAGCAGCTGCCTGGCACAGCCCCAAAGCTGCT |
| | | | GATCTACTATAATGACCAGAGACCCTCCGGCGTGCCTGATAGATTCTCT |
| | | | GGCAGCAAGTCCGGCACCTCTGCCAGCCTGGCCATCACAGGACTGCAG |
| | | | GCAGAGGACGAGGCAGATTACTATTGTCAGTCCTACGACAGATATACC |
| | | | CACCCAGCCCTGCTGTTTGGCACCGGCACAAAGGTGACAGTGCTGGCCG |
| | | | CCGAGCCCAAGTCCTCTGATAAGACCCACACATGCCCACCCTGTCCGGC |
| | | | GCCAGAGGCAGCAGGAGGACCAAGCGTGTTCCTGTTTCCACCCAAGCC |
| | | | CAAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAACCACAGGTCTACGTGCTGCCTCCATCCCGGGACGAGCTGACAAAG |
| | | | AACCAGGTCTCTCTGCTGTGCCTGGTGAAAGGCTTCTATCCATCAGATA |
| | | | TTGCTGTGGAGTGGGAAAGCAATGGGCAGCCCGAGAACAATTACCTGA |
| | | | CTTGGCCCCCTGTGCTGGACTCTGATGGGAGTTTCTTTCTGTATTCTAAG |
| | | | CTGACCGTGGATAAAAGTAGGTGGCAGCAGGGAAATGTCTTTAGTTGTT |
| | | | CAGTGATGCATGAAGCCCTGCATAACCACTACACCCAGAAAAGCCTGT |
| | | | CCCTGTCCCCCGGA |
| 138 | 22209 | Full nt | CAGGTGCAGCTGGTGGAGAGCGGCGGCGGCGTGGTGCAGCCTGGCCGG |
| | | | TCCCTGAGACTGTCTTGCGCAGCCAGCGGCTTCACCTTTAGCTCCTACG |
| | | | GCATGCACTGGGTGCGGCAGGCCCCTGGCAAGGGACTGGAGTGGGTGG |
| | | | CCTTCATCGAGGTGGACGGCTCTAACAAGTACTATGCCGATAGCGTGAA |
| | | | GGGCAGGTTTACCATCTCCCGCGACAACTCTAAGAATACACTGTACCTG |
| | | | CAGATGAACTCCCTGCGGGCAGAGGACACCGCCGTGTACTATTGCAAG |
| | | | ACACACGGCTCTCACGATAATTGGGGCCAGGGCACCATGGTGACAGTG |
| | | | TCTAGCGGAGGAGGAGGCAGCGGAGGAGGAGGCTCCGGCGGCGGCGG |
| | | | CAGCCAGTCCGTGCTGACCCAGCCACCTTCTGTGAGCGGAGCCCCCGGA |
| | | | CAGAGGGTGACAATCTCCTGTTCTGGCAGCCGCTCCAACATCGGCAGCA |
| | | | ATACCGTGAAGTGGTATCAGCAGCTGCCTGGCACAGCCCCAAAGCTGCT |
| | | | GATCTACTATAATGACCAGAGACCCTCCGGCGTGCCTGATAGATTCTCT |
| | | | GGCAGCAAGTCCGGCACCTCTGCCAGCCTGGCCATCACAGGACTGCAG |
| | | | GCAGAGGACGAGGCAGATTACTATTGTCAGTCCTACGACAGATATACC |
| | | | CACCCAGCCCTGCTGTTTGGCACCGGCACAAAGGTGACAGTGCTGGCCG |
| | | | CCGAGCCCAAGTCCTCTGATAAGACCCACACATGCCCACCCTGTCCGGC |
| | | | GCCAGAGGCAGCAGGAGGACCAAGCGTGTTCCTGTTTCCACCCAAGCC |
| | | | CAAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAACCACAGGTCTACGTGCTGCCTCCATCCCGGGACGAGCTGACAAAG |
| | | | AACCAGGTCTCTCTGCTGTGCCTGGTGAAAGGCTTCTATCCATCAGATA |
| | | | TTGCTGTGGAGTGGGAAAGCAATGGGCAGCCCGAGAACAATTACCTGA |
| | | | CTTGGCCCCCTGTGCTGGACTCTGATGGGAGTTTCTTTCTGTATTCTAAG |
| | | | CTGACCGTGGATAAAAGTAGGTGGCAGCAGGGAAATGTCTTTAGTTGTT |
| | | | CAGTGATGCATGAAGCCCTGCATAACCACTACACCCAGAAAAGCCTGT |
| | | | CCCTGTCCCCCGGA |
| 139 | 22211 | Full nt | CAGGTGCAGCTGGTGGAGAGCGGCGGCGGCGTGGTGCAGCCTGGCCGG |
| | | | TCCCTGAGACTGTCTTGCGCAGCCAGCGGCTTCACCTTTAGCTCCTACG |
| | | | GCATGCACTGGGTGCGGCAGGCCCCTGGCAAGGGACTGGAGTGGGTGG |
| | | | CCTTCATCAGATATGACGGCTCTAACAAGTACTATGCCGATAGCGTGAA |
| | | | GGGCAGGTTTACCATCTCCCGCGACAACTCTAAGAATACACTGTACCTG |
| | | | CAGATGAACTCCCTGCGGGCAGAGGACACCGCCGTGTACTATTGCAAG |
| | | | ACAGACGGCTCTCACGATAATTGGGGCCAGGGCACCATGGTGACAGTG |
| | | | TCTAGCGGAGGAGGAGGCAGCGGAGGAGGAGGCTCCGGCGGCGGCGG |
| | | | CAGCCAGTCCGTGCTGACCCAGCCACCTTCTGTGAGCGGAGCCCCCGGA |
| | | | CAGAGGGTGACAATCTCCTGTTCTGGCAGCCGCTCCAACATCGGCAGCA |
| | | | ATACCGTGAAGTGGTATCAGCAGCTGCCTGGCACAGCCCCAAAGCTGCT |
| | | | GATCTACTATAATGACCAGAGACCCTCCGGCGTGCCTGATAGATTCTCT |
| | | | GGCAGCAAGTCCGGCACCTCTGCCAGCCTGGCCATCACAGGACTGCAG |
| | | | GCAGAGGACGAGGCAGATTACTATTGTCAGTCCTACGACAGATATACC |
| | | | CACCCAGCCCTGCTGTTTGGCACCGGCACAAAGGTGACAGTGCTGGCCG |
| | | | CCGAGCCCAAGTCCTCTGATAAGACCCACACATGCCCACCCTGTCCGGC |
| | | | GCCAGAGGCAGCAGGAGGACCAAGCGTGTTCCTGTTTCCACCCAAGCC |
| | | | CAAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAACCACAGGTCTACGTGCTGCCTCCATCCCGGGACGAGCTGACAAAG |
| | | | AACCAGGTCTCTCTGCTGTGCCTGGTGAAAGGCTTCTATCCATCAGATA |
| | | | TTGCTGTGGAGTGGGAAAGCAATGGGCAGCCCGAGAACAATTACCTGA |
| | | | CTTGGCCCCCTGTGCTGGACTCTGATGGGAGTTTCTTTCTGTATTCTAAG |
| | | | CTGACCGTGGATAAAAGTAGGTGGCAGCAGGGAAATGTCTTTAGTTGTT |
| | | | CAGTGATGCATGAAGCCCTGCATAACCACTACACCCAGAAAAGCCTGT |
| | | | CCCTGTCCCCCGGA |
| 140 | 22212 | Full nt | CAGGTGCAGCTGGTGGAGAGCGGCGGCGGCGTGGTGCAGCCTGGCCGG |
| | | | TCCCTGAGACTGTCTTGCGCAGCCAGCGGCTTCACCTTTAGCTCCTACG |
| | | | GCATGCACTGGGTGCGGCAGGCCCCTGGCAAGGGACTGGAGTGGGTGG |
| | | | CCTTCATCAGATATGACGGCTCTAACAAGTACTATGCCGATAGCGTGAA |
| | | | GGGCAGGTTTACCATCTCCCGCGACAACTCTAAGAATACACTGTACCTG |
| | | | CAGATGAACTCCCTGCGGGCAGAGGACACCGCCGTGTACTATTGCAAG |
| | | | ACACACACCTCTCACGATAATTGGGGCCAGGGCACCATGGTGACAGTG |
| | | | TCTAGCGGAGGAGGAGGCAGCGGAGGAGGAGGCTCCGGCGGCGGCGG |
| | | | CAGCCAGTCCGTGCTGACCCAGCCACCTTCTGTGAGCGGAGCCCCCGGA |
| | | | CAGAGGGTGACAATCTCCTGTTCTGGCAGCCGCTCCAACATCGGCAGCA |
| | | | ATACCGTGAAGTGGTATCAGCAGCTGCCTGGCACAGCCCCAAAGCTGCT |
| | | | GATCTACTATAATGACCAGAGACCCTCCGGCGTGCCTGATAGATTCTCT |
| | | | GGCAGCAAGTCCGGCACCTCTGCCAGCCTGGCCATCACAGGACTGCAG |
| | | | GCAGAGGACGAGGCAGATTACTATTGTCAGTCCTACGACAGATATACC |
| | | | CACCCAGCCCTGCTGTTTGGCACCGGCACAAAGGTGACAGTGCTGGCCG |
| | | | CCGAGCCCAAGTCCTCTGATAAGACCCACACATGCCCACCCTGTCCGGC |
| | | | GCCAGAGGCAGCAGGAGGACCAAGCGTGTTCCTGTTTCCACCCAAGCC |
| | | | CAAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAACCACAGGTCTACGTGCTGCCTCCATCCCGGGACGAGCTGACAAAG |
| | | | AACCAGGTCTCTCTGCTGTGCCTGGTGAAAGGCTTCTATCCATCAGATA |
| | | | TTGCTGTGGAGTGGGAAAGCAATGGGCAGCCCGAGAACAATTACCTGA |
| | | | CTTGGCCCCCTGTGCTGGACTCTGATGGGAGTTTCTTTCTGTATTCTAAG |
| | | | CTGACCGTGGATAAAAGTAGGTGGCAGCAGGGAAATGTCTTTAGTTGTT |
| | | | CAGTGATGCATGAAGCCCTGCATAACCACTACACCCAGAAAAGCCTGT |
| | | | CCCTGTCCCCCGGA |
| 141 | 22214 | Full nt | CAGGTGCAGCTGGTGGAGAGCGGCGGCGGCGTGGTGCAGCCTGGCCGG |
| | | | TCCCTGAGACTGTCTTGCGCAGCCAGCGGCTTCACCTTTAGCTCCTACG |
| | | | GCATGCACTGGGTGCGGCAGGCCCCTGGCAAGGGACTGGAGTGGGTGG |
| | | | CCTTCATCAGATATGACGGCTCTAACAAGTACTATGCCGATAGCGTGAA |
| | | | GGGCAGGTTTACCATCTCCCGCGACAACTCTAAGAATACACTGTACCTG |
| | | | CAGATGAACTCCCTGCGGGCAGAGGACACCGCCGTGTACTATTGCAAG |
| | | | ACACACGGCTCTGCCGATAATTGGGGCCAGGGCACCATGGTGACAGTG |
| | | | TCTAGCGGAGGAGGAGGCAGCGGAGGAGGAGGCTCCGGCGGCGGCGG |
| | | | CAGCCAGTCCGTGCTGACCCAGCCACCTTCTGTGAGCGGAGCCCCCGGA |
| | | | CAGAGGGTGACAATCTCCTGTTCTGGCAGCCGCTCCAACATCGGCAGCA |
| | | | ATACCGTGAAGTGGTATCAGCAGCTGCCTGGCACAGCCCCAAAGCTGCT |
| | | | GATCTACTATAATGACCAGAGACCCTCCGGCGTGCCTGATAGATTCTCT |
| | | | GGCAGCAAGTCCGGCACCTCTGCCAGCCTGGCCATCACAGGACTGCAG |
| | | | GCAGAGGACGAGGCAGATTACTATTGTCAGTCCTACGACAGATATACC |
| | | | CACCCAGCCCTGCTGTTTGGCACCGGCACAAAGGTGACAGTGCTGGCCG |
| | | | CCGAGCCCAAGTCCTCTGATAAGACCCACACATGCCCACCCTGTCCGGC |
| | | | GCCAGAGGCAGCAGGAGGACCAAGCGTGTTCCTGTTTCCACCCAAGCC |
| | | | CAAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAACCACAGGTCTACGTGCTGCCTCCATCCCGGGACGAGCTGACAAAG |
| | | | AACCAGGTCTCTCTGCTGTGCCTGGTGAAAGGCTTCTATCCATCAGATA |
| | | | TTGCTGTGGAGTGGGAAAGCAATGGGCAGCCCGAGAACAATTACCTGA |
| | | | CTTGGCCCCCTGTGCTGGACTCTGATGGGAGTTTCTTTCTGTATTCTAAG |
| | | | CTGACCGTGGATAAAAGTAGGTGGCAGCAGGGAAATGTCTTTAGTTGTT |
| | | | CAGTGATGCATGAAGCCCTGCATAACCACTACACCCAGAAAAGCCTGT |
| | | | CCCTGTCCCCCGGA |
| 142 | 22279 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAAGTCTAAGAGGGAGAAG |
| | | | AAGGACCGCGTGTTCACCGATAAGACAAGCGCCACCGTGATCTGTCGG |
| | | | AAGAACGCCAGCATCTCCGTGCGGGCCCAGGATAGATACTATTCTAGCT |
| | | | CCTGGAGCGAGTGGGCCTCCGTGCCATGTTCTGGAGGAGGAGGCAGCG |
| | | | GCGGAGGAGGCTCCGGCGGCGGCGGCTCTAATCTGCCAGTGGCCACCC |
| | | | CTGACCCAGGCATGTTCCCCTGCCTGCACCACTCTCAGAACCTGCTGCG |
| | | | GGCCGTGAGCAATATGCTGCAGAAGGCCAGACAGACACTGGAGTTTTA |
| | | | CCCATGTACCTCCGAGGAGATCGACCACGAGGATATCACAAAGGATAA |
| | | | GACCTCTACAGTGGAGGCATGCCTGCCACTGGAGCTGACCAAGAACGA |
| | | | GAGCTGTCTGAACAGCCGGGAGACATCTTTCATCACCAACGGCAGCTGC |
| | | | CTGGCCTCCAGAAAGACATCTTTTATGATGGCCCTGTGCCTGTCTAGCA |
| | | | TCTACGAGGACCTGAAGATGTATCAGGTGGAGTTCAAGACCATGAACG |
| | | | CCAAGCTGCTGATGGACCCTAAGAGGCAGATCTTTCTGGATCAGAATAT |
| | | | GCTGGCCGTGATCGACGAGCTGATGCAGGCCCTGAACTTCAATAGCGA |
| | | | GACAGTGCCACAGAAGTCCTCTCTGGAGGAGCCCGATTTCTACAAGACC |
| | | | AAGATCAAGCTGTGCATCCTGCTGCACGCCTTCCGGATCAGAGCCGTGA |
| | | | CCATCGACCGCGTGATGAGCTACCTGAACGCCAGC |
| 143 | 22289 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGAGAGACGACTCTGAGGACCGCGTGT |
| | | | TCACCGATAAGACAAGCGCCACCGTGATCTGTCGGAAGAACGCCAGCA |
| | | | TCTCCGTGCGGGCCCAGGATAGATACTATTCTAGCTCCTGGAGCGAGTG |
| | | | GGCCTCCGTGCCATGTTCTGGAGGAGGAGGCAGCGGCGGAGGAGGCTC |
| | | | CGGCGGCGGCGGCTCTAATCTGCCAGTGGCCACCCCTGACCCAGGCATG |
| | | | TTCCCCTGCCTGCACCACTCTCAGAACCTGCTGCGGGCCGTGAGCAATA |
| | | | TGCTGCAGAAGGCCAGACAGACACTGGAGTTTTACCCATGTACCTCCGA |
| | | | GGAGATCGACCACGAGGATATCACAAAGGATAAGACCTCTACAGTGGA |
| | | | GGCATGCCTGCCACTGGAGCTGACCAAGAACGAGAGCTGTCTGAACAG |
| | | | CCGGGAGACATCTTTCATCACCAACGGCAGCTGCCTGGCCTCCAGAAAG |
| | | | ACATCTTTTATGATGGCCCTGTGCCTGTCTAGCATCTACGAGGACCTGA |
| | | | AGATGTATCAGGTGGAGTTCAAGACCATGAACGCCAAGCTGCTGATGG |
| | | | ACCCTAAGAGGCAGATCTTTCTGGATCAGAATATGCTGGCCGTGATCGA |
| | | | CGAGCTGATGCAGGCCCTGAACTTCAATAGCGAGACAGTGCCACAGAA |
| | | | GTCCTCTCTGGAGGAGCCCGATTTCTACAAGACCAAGATCAAGCTGTGC |
| | | | ATCCTGCTGCACGCCTTCCGGATCAGAGCCGTGACCATCGACCGCGTGA |
| | | | TGAGCTACCTGAACGCCAGC |
| 144 | 22290 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAGCCAGGAGAAGAAGGAC |
| | | | CGCGTGTTCACCGATAAGACAAGCGCCACCGTGATCTGTCGGAAGAAC |
| | | | GCCAGCATCTCCGTGCGGGCCCAGGATAGATACTATTCTAGCTCCTGGA |
| | | | GCGAGTGGGCCTCCGTGCCATGTTCTGGAGGAGGAGGCAGCGGCGGAG |
| | | | GAGGCTCCGGCGGCGGCGGCTCTAATCTGCCAGTGGCCACCCCTGACCC |
| | | | AGGCATGTTCCCCTGCCTGCACCACTCTCAGAACCTGCTGCGGGCCGTG |
| | | | AGCAATATGCTGCAGAAGGCCAGACAGACACTGGAGTTTTACCCATGT |
| | | | ACCTCCGAGGAGATCGACCACGAGGATATCACAAAGGATAAGACCTCT |
| | | | ACAGTGGAGGCATGCCTGCCACTGGAGCTGACCAAGAACGAGAGCTGT |
| | | | CTGAACAGCCGGGAGACATCTTTCATCACCAACGGCAGCTGCCTGGCCT |
| | | | CCAGAAAGACATCTTTTATGATGGCCCTGTGCCTGTCTAGCATCTACGA |
| | | | GGACCTGAAGATGTATCAGGTGGAGTTCAAGACCATGAACGCCAAGCT |
| | | | GCTGATGGACCCTAAGAGGCAGATCTTTCTGGATCAGAATATGCTGGCC |
| | | | GTGATCGACGAGCTGATGCAGGCCCTGAACTTCAATAGCGAGACAGTG |
| | | | CCACAGAAGTCCTCTCTGGAGGAGCCCGATTTCTACAAGACCAAGATCA |
| | | | AGCTGTGCATCCTGCTGCACGCCTTCCGGATCAGAGCCGTGACCATCGA |
| | | | CCGCGTGATGAGCTACCTGAACGCCAGC |
| 145 | 22291 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCGAGTCTAAGCAGGAGAAG |
| | | | AAGGACCGCGTGTTCACCGATAAGACAAGCGCCACCGTGATCTGTCGG |
| | | | AAGAACGCCAGCATCTCCGTGCGGGCCCAGGATAGATACTATTCTAGCT |
| | | | CCTGGAGCGAGTGGGCCTCCGTGCCATGTTCTGGAGGAGGAGGCAGCG |
| | | | GCGGAGGAGGCTCCGGCGGCGGCGGCTCTAATCTGCCAGTGGCCACCC |
| | | | CTGACCCAGGCATGTTCCCCTGCCTGCACCACTCTCAGAACCTGCTGCG |
| | | | GGCCGTGAGCAATATGCTGCAGAAGGCCAGACAGACACTGGAGTTTTA |
| | | | CCCATGTACCTCCGAGGAGATCGACCACGAGGATATCACAAAGGATAA |
| | | | GACCTCTACAGTGGAGGCATGCCTGCCACTGGAGCTGACCAAGAACGA |
| | | | GAGCTGTCTGAACAGCCGGGAGACATCTTTCATCACCAACGGCAGCTGC |
| | | | CTGGCCTCCAGAAAGACATCTTTTATGATGGCCCTGTGCCTGTCTAGCA |
| | | | TCTACGAGGACCTGAAGATGTATCAGGTGGAGTTCAAGACCATGAACG |
| | | | CCAAGCTGCTGATGGACCCTAAGAGGCAGATCTTTCTGGATCAGAATAT |
| | | | GCTGGCCGTGATCGACGAGCTGATGCAGGCCCTGAACTTCAATAGCGA |
| | | | GACAGTGCCACAGAAGTCCTCTCTGGAGGAGCCCGATTTCTACAAGACC |
| | | | AAGATCAAGCTGTGCATCCTGCTGCACGCCTTCCGGATCAGAGCCGTGA |
| | | | CCATCGACCGCGTGATGAGCTACCTGAACGCCAGC |
| 146 | 22292 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCGAGAAGAAGGACCGCGTGT |
| | | | TCACCGATAAGACAAGCGCCACCGTGATCTGTCGGAAGAACGCCAGCA |
| | | | TCTCCGTGCGGGCCCAGGATAGATACTATTCTAGCTCCTGGAGCGAGTG |
| | | | GGCCTCCGTGCCATGTTCTGGAGGAGGAGGCAGCGGCGGAGGAGGCTC |
| | | | CGGCGGCGGCGGCTCTAATCTGCCAGTGGCCACCCCTGACCCAGGCATG |
| | | | TTCCCCTGCCTGCACCACTCTCAGAACCTGCTGCGGGCCGTGAGCAATA |
| | | | TGCTGCAGAAGGCCAGACAGACACTGGAGTTTTACCCATGTACCTCCGA |
| | | | GGAGATCGACCACGAGGATATCACAAAGGATAAGACCTCTACAGTGGA |
| | | | GGCATGCCTGCCACTGGAGCTGACCAAGAACGAGAGCTGTCTGAACAG |
| | | | CCGGGAGACATCTTTCATCACCAACGGCAGCTGCCTGGCCTCCAGAAAG |
| | | | ACATCTTTTATGATGGCCCTGTGCCTGTCTAGCATCTACGAGGACCTGA |
| | | | AGATGTATCAGGTGGAGTTCAAGACCATGAACGCCAAGCTGCTGATGG |
| | | | ACCCTAAGAGGCAGATCTTTCTGGATCAGAATATGCTGGCCGTGATCGA |
| | | | CGAGCTGATGCAGGCCCTGAACTTCAATAGCGAGACAGTGCCACAGAA |
| | | | GTCCTCTCTGGAGGAGCCCGATTTCTACAAGACCAAGATCAAGCTGTGC |
| | | | ATCCTGCTGCACGCCTTCCGGATCAGAGCCGTGACCATCGACCGCGTGA |
| | | | TGAGCTACCTGAACGCCAGC |
| 147 | 22293 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGACCAGACCGACGACCGCGTGT |
| | | | TCACCGATAAGACAAGCGCCACCGTGATCTGTCGGAAGAACGCCAGCA |
| | | | TCTCCGTGCGGGCCCAGGATAGATACTATTCTAGCTCCTGGAGCGAGTG |
| | | | GGCCTCCGTGCCATGTTCTGGAGGAGGAGGCAGCGGCGGAGGAGGCTC |
| | | | CGGCGGCGGCGGCTCTAATCTGCCAGTGGCCACCCCTGACCCAGGCATG |
| | | | TTCCCCTGCCTGCACCACTCTCAGAACCTGCTGCGGGCCGTGAGCAATA |
| | | | TGCTGCAGAAGGCCAGACAGACACTGGAGTTTTACCCATGTACCTCCGA |
| | | | GGAGATCGACCACGAGGATATCACAAAGGATAAGACCTCTACAGTGGA |
| | | | GGCATGCCTGCCACTGGAGCTGACCAAGAACGAGAGCTGTCTGAACAG |
| | | | CCGGGAGACATCTTTCATCACCAACGGCAGCTGCCTGGCCTCCAGAAAG |
| | | | ACATCTTTTATGATGGCCCTGTGCCTGTCTAGCATCTACGAGGACCTGA |
| | | | AGATGTATCAGGTGGAGTTCAAGACCATGAACGCCAAGCTGCTGATGG |
| | | | ACCCTAAGAGGCAGATCTTTCTGGATCAGAATATGCTGGCCGTGATCGA |
| | | | CGAGCTGATGCAGGCCCTGAACTTCAATAGCGAGACAGTGCCACAGAA |
| | | | GTCCTCTCTGGAGGAGCCCGATTTCTACAAGACCAAGATCAAGCTGTGC |
| | | | ATCCTGCTGCACGCCTTCCGGATCAGAGCCGTGACCATCGACCGCGTGA |
| | | | TGAGCTACCTGAACGCCAGC |
| 148 | 22294 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGACGACTCTGAGGACCGCGTGT |
| | | | TCACCGATAAGACAAGCGCCACCGTGATCTGTCGGAAGAACGCCAGCA |
| | | | TCTCCGTGCGGGCCCAGGATAGATACTATTCTAGCTCCTGGAGCGAGTG |
| | | | GGCCTCCGTGCCATGTTCTGGAGGAGGAGGCAGCGGCGGAGGAGGCTC |
| | | | CGGCGGCGGCGGCTCTAATCTGCCAGTGGCCACCCCTGACCCAGGCATG |
| | | | TTCCCCTGCCTGCACCACTCTCAGAACCTGCTGCGGGCCGTGAGCAATA |
| | | | TGCTGCAGAAGGCCAGACAGACACTGGAGTTTTACCCATGTACCTCCGA |
| | | | GGAGATCGACCACGAGGATATCACAAAGGATAAGACCTCTACAGTGGA |
| | | | GGCATGCCTGCCACTGGAGCTGACCAAGAACGAGAGCTGTCTGAACAG |
| | | | CCGGGAGACATCTTTCATCACCAACGGCAGCTGCCTGGCCTCCAGAAAG |
| | | | ACATCTTTTATGATGGCCCTGTGCCTGTCTAGCATCTACGAGGACCTGA |
| | | | AGATGTATCAGGTGGAGTTCAAGACCATGAACGCCAAGCTGCTGATGG |
| | | | ACCCTAAGAGGCAGATCTTTCTGGATCAGAATATGCTGGCCGTGATCGA |
| | | | CGAGCTGATGCAGGCCCTGAACTTCAATAGCGAGACAGTGCCACAGAA |
| | | | GTCCTCTCTGGAGGAGCCCGATTTCTACAAGACCAAGATCAAGCTGTGC |
| | | | ATCCTGCTGCACGCCTTCCGGATCAGAGCCGTGACCATCGACCGCGTGA |
| | | | TGAGCTACCTGAACGCCAGC |
| 149 | 22295 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGAAGGACCAGACCGAGGACCGCGTGT |
| | | | TCACCGATAAGACAAGCGCCACCGTGATCTGTCGGAAGAACGCCAGCA |
| | | | TCTCCGTGCGGGCCCAGGATAGATACTATTCTAGCTCCTGGAGCGAGTG |
| | | | GGCCTCCGTGCCATGTTCTGGAGGAGGAGGCAGCGGCGGAGGAGGCTC |
| | | | CGGCGGCGGCGGCTCTAATCTGCCAGTGGCCACCCCTGACCCAGGCATG |
| | | | TTCCCCTGCCTGCACCACTCTCAGAACCTGCTGCGGGCCGTGAGCAATA |
| | | | TGCTGCAGAAGGCCAGACAGACACTGGAGTTTTACCCATGTACCTCCGA |
| | | | GGAGATCGACCACGAGGATATCACAAAGGATAAGACCTCTACAGTGGA |
| | | | GGCATGCCTGCCACTGGAGCTGACCAAGAACGAGAGCTGTCTGAACAG |
| | | | CCGGGAGACATCTTTCATCACCAACGGCAGCTGCCTGGCCTCCAGAAAG |
| | | | ACATCTTTTATGATGGCCCTGTGCCTGTCTAGCATCTACGAGGACCTGA |
| | | | AGATGTATCAGGTGGAGTTCAAGACCATGAACGCCAAGCTGCTGATGG |
| | | | ACCCTAAGAGGCAGATCTTTCTGGATCAGAATATGCTGGCCGTGATCGA |
| | | | CGAGCTGATGCAGGCCCTGAACTTCAATAGCGAGACAGTGCCACAGAA |
| | | | GTCCTCTCTGGAGGAGCCCGATTTCTACAAGACCAAGATCAAGCTGTGC |
| | | | ATCCTGCTGCACGCCTTCCGGATCAGAGCCGTGACCATCGACCGCGTGA |
| | | | TGAGCTACCTGAACGCCAGC |
| 150 | 22296 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAGCGAGAAGGACCGCGTGT |
| | | | TCACCGATAAGACAAGCGCCACCGTGATCTGTCGGAAGAACGCCAGCA |
| | | | TCTCCGTGCGGGCCCAGGATAGATACTATTCTAGCTCCTGGAGCGAGTG |
| | | | GGCCTCCGTGCCATGTTCTGGAGGAGGAGGCAGCGGCGGAGGAGGCTC |
| | | | CGGCGGCGGCGGCTCTAATCTGCCAGTGGCCACCCCTGACCCAGGCATG |
| | | | TTCCCCTGCCTGCACCACTCTCAGAACCTGCTGCGGGCCGTGAGCAATA |
| | | | TGCTGCAGAAGGCCAGACAGACACTGGAGTTTTACCCATGTACCTCCGA |
| | | | GGAGATCGACCACGAGGATATCACAAAGGATAAGACCTCTACAGTGGA |
| | | | GGCATGCCTGCCACTGGAGCTGACCAAGAACGAGAGCTGTCTGAACAG |
| | | | CCGGGAGACATCTTTCATCACCAACGGCAGCTGCCTGGCCTCCAGAAAG |
| | | | ACATCTTTTATGATGGCCCTGTGCCTGTCTAGCATCTACGAGGACCTGA |
| | | | AGATGTATCAGGTGGAGTTCAAGACCATGAACGCCAAGCTGCTGATGG |
| | | | ACCCTAAGAGGCAGATCTTTCTGGATCAGAATATGCTGGCCGTGATCGA |
| | | | CGAGCTGATGCAGGCCCTGAACTTCAATAGCGAGACAGTGCCACAGAA |
| | | | GTCCTCTCTGGAGGAGCCCGATTTCTACAAGACCAAGATCAAGCTGTGC |
| | | | ATCCTGCTGCACGCCTTCCGGATCAGAGCCGTGACCATCGACCGCGTGA |
| | | | TGAGCTACCTGAACGCCAGC |
| 151 | 22672 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCAGGAGGAGGAGGAGGCTCTATGAGCGGCAGGAGCGCCA |
| | | | ATGCCGGAGGAGGAGGCAGCGGAGGAGGAGGCTCCGGAGGCGGCGGC |
| | | | TCTGGCGGCGGCGGCAGCAAGATCGACGCCTGCAAGCGCGGCGATGTG |
| | | | ACAGTGAAGCCTAGCCACGTGATCCTGCTGGGCTCCACCGTGAATATCA |
| | | | CATGCTCTCTGAAGCCACGGCAGGGCTGTTTCCACTACTCCCGGAGAAA |
| | | | CAAGCTGATCCTGTATAAGTTCGACAGGCGCATCAACTTTCACCACGGC |
| | | | CACTCTCTGAATAGCCAGGTGACCGGACTGCCACTGGGCACCACACTGT |
| | | | TCGTGTGCAAGCTGGCCTGTATCAATAGCGATGAGATCCAGATCTGTGG |
| | | | AGCCGAGATCTTTGTGGGAGTGGCCCCCGAGCAGCCTCAGAACCTGTCC |
| | | | TGCATCCAGAAGGGAGAGCAGGGAACCGTGGCATGTACATGGGAGCGG |
| | | | GGCAGAGACACCCACCTGTACACCGAGTATACACTGCAGCTGAGCGGC |
| | | | CCCAAGAACCTGACATGGCAGAAGCAGTGCAAGGATATCTACTGTGAC |
| | | | TATCTGGATTTCGGCATCAACCTGACCCCAGAGTCCCCCGAGTCTAACT |
| | | | TCACCGCCAAGGTGACAGCCGTGAACTCTCTGGGCAGCTCCTCTAGCCT |
| | | | GCCCAGCACCTTCACATTTCTGGACATCGTGCGCCCTCTGCCTCCATGG |
| | | | GATATCCGGATCAAGTTTCAGAAGGCCTCCGTGTCTAGATGCACACTGT |
| | | | ACTGGAGGGACGAGGGCCTGGTGCTGCTGAACAGGCTGCGCTATAGAC |
| | | | CCAGCAATTCCAGACTGTGGAACATGGTGAATGTGACCAAGGCCAAGG |
| | | | GCAGGCACGACCTGCTGGATCTGAAGCCTTTCACAGAGTACGAGTTTCA |
| | | | GATCTCCTCTAAGCTGCACCTGTATAAGGGCTCTTGGAGCGATTGGTCC |
| | | | GAGTCTCTGAGGGCACAGACCCCTGAGGAGGAGCCA |
| 152 | 22735 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGAGGAGGCTCTGGAGGAGGAGGCTCCATGA |
| | | | GCGGGCGGAGCGCCAACGCAGGGGGCGGCGGCTCTGGCGGCGGCGGC |
| | | | AGCCAGGTGCAGCTGGTGGAGAGCGGCGGCGGCGTGGTGCAGCCCGGC |
| | | | AGAAGCCTGCGGCTGAGCTGCGCAGCCTCTGGCTTCACCTTTAGCTCCT |
| | | | ACGGCATGCACTGGGTGCGGCAGGCCCCTGGCAAGGGACTGGAGTGGG |
| | | | TGGCCTTCATCAGATATGACGGCTCCAATAAGTACTATGCCGATTCTGT |
| | | | GAAGGGCAGGTTTACCATCAGCCGCGACAACTCCAAGAATACACTGTA |
| | | | CCTGCAGATGAACTCCCTGAGGGCCGAGGACACCGCCGTGTACTATTGC |
| | | | AAGACACACGGCTCTCACGATAATTGGGGCCAGGGCACCATGGTGACA |
| | | | GTGTCTTCCGGAGGAGGAGGCAGCATGAGCGGGCGGAGCGCCAACGCA |
| | | | GGGGGTGGAGGCTCCGGAGGAGGAGGCTCTCAGAGCGTGCTGACCCAG |
| | | | CCACCTTCCGTGTCTGGAGCCCCCGGACAGAGGGTGACAATCAGCTGTT |
| | | | CCGGCTCTCGCAGCAACATCGGCAGCAATACCGTGAAGTGGTATCAGC |
| | | | AGCTGCCAGGCACAGCCCCCAAGCTGCTGATCTACTATAATGACCAGCG |
| | | | GCCTTCCGGCGTGCCAGATAGATTCTCCGGCTCTAAGAGCGGCACCTCC |
| | | | GCCTCTCTGGCCATCACAGGCCTGCAGGCAGAGGACGAGGCAGATTAC |
| | | | TATTGTCAGTCCTACGATAGATATACCCACCCCGCCCTGCTGTTTGGCA |
| | | | CCGGCACAAAGGTGACAGTGCTG |
| 153 | 23360 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCAGGAGGAGGAGGAGGCTCCGGCGGAGGAGGCTCCATGA |
| | | | GCGGGCGGAGCGCCAACGCAGGGGGCGGCGGCTCTGGCGGCGGCGGC |
| | | | AGCCAGTCCGTGCTGACCCAGCCACCTTCTGTGAGCGGAGCCCCCGGAC |
| | | | AGAGGGTGACAATCTCCTGCTCTGGCAGCCGCTCCAACATCGGCTCTAA |
| | | | TACCGTGAAGTGGTATCAGCAGCTGCCAGGCACAGCCCCCAAGCTGCT |
| | | | GATCTACTATAATGACCAGCGGCCTAGCGGCGTGCCAGATAGATTCTCT |
| | | | GGCAGCAAGTCCGGCACCTCTGCCAGCCTGGCCATCACAGGCCTGCAG |
| | | | GCAGAGGACGAGGCAGATTACTATTGCCAGTCCTACGACCGCTATACCC |
| | | | ACCCTGCCCTGCTGTTTGGCACCGGCACAAAGGTGACAGTGCTGGGCGG |
| | | | CTCCGGCGGCGGCTCTGGAGGAGGCAGCGGCGGCGGCTCCGGAGGAGG |
| | | | CTCTGGCCAGGTGCAGCTGGTGGAGAGCGGAGGAGGAGTGGTGCAGCC |
| | | | CGGCAGAAGCCTGCGGCTGAGCTGTGCAGCCTCTGGCTTCACCTTTAGC |
| | | | TCCTACGGCATGCACTGGGTGCGGCAGGCCCCTGGCAAGGGACTGGAG |
| | | | TGGGTGGCCTTCATCAGATATGACGGCTCTAACAAGTACTATGCCGATA |
| | | | GCGTGAAGGGCAGGTTTACCATCAGCCGCGATAACTCCAAGAATACAC |
| | | | TGTATCTGCAGATGAACAGCCTGCGGGCAGAGGACACCGCCGTGTACT |
| | | | ATTGTAAGACACACGGCTCCCACGATAATTGGGGCCAGGGCACCATGG |
| | | | TGACAGTGTCTAGC |
| 154 | 23361 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCAGGAGGAGGAGGAGGCTCCGGCGGAGGAGGCTCCATGA |
| | | | GCGGGCGGAGCGCCAACGCAGGGGGCGGCGGCTCTGGCGGCGGCGGC |
| | | | AGCCAGTCCGTGCTGACCCAGCCACCTTCTGTGAGCGGAGCCCCCGGAC |
| | | | AGAGGGTGACAATCTCCTGCTCTGGCAGCCGCTCCAACATCGGCTCTAA |
| | | | TACCGTGAAGTGGTATCAGCAGCTGCCAGGCACAGCCCCCAAGCTGCT |
| | | | GATCTACTATAATGACCAGCGGCCTAGCGGCGTGCCAGATAGATTCTCT |
| | | | GGCAGCAAGTCCGGCACCTCTGCCAGCCTGGCCATCACAGGCCTGCAG |
| | | | GCAGAGGACGAGGCAGATTACTATTGCCAGTCCTACGACCGCTATACCC |
| | | | ACCCTGCCCTGCTGTTTGGCTGCGGCACAAAGGTGACAGTGCTGGGCGG |
| | | | CTCCGGCGGCGGCTCTGGAGGAGGCAGCGGCGGCGGCTCCGGAGGAGG |
| | | | CTCTGGCCAGGTGCAGCTGGTGGAGAGCGGAGGAGGAGTGGTGCAGCC |
| | | | CGGCAGAAGCCTGCGGCTGAGCTGTGCAGCCTCTGGCTTCACCTTTAGC |
| | | | TCCTACGGCATGCACTGGGTGCGGCAGGCCCCTGGCAAGTGCCTGGAGT |
| | | | GGGTGGCCTTCATCAGATATGACGGCTCTAACAAGTACTATGCCGATAG |
| | | | CGTGAAGGGCAGGTTTACCATCAGCCGCGATAACTCCAAGAATACACT |
| | | | GTATCTGCAGATGAACAGCCTGCGGGCAGAGGACACCGCCGTGTACTA |
| | | | TTGTAAGACACACGGCTCCCACGATAATTGGGGCCAGGGCACCATGGT |
| | | | GACAGTGTCTAGC |
| 155 | 23363 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGAGGAGGCTCCGGCGGAGGAGGCTCCATGA |
| | | | GCGGGCGGAGCGCCAACGCAGGGGGCGGCGGCTCCGGCGGCGGCGGCT |
| | | | CTCAGGTGCAGCTGGTGGAGAGCGGAGGAGGAGTGGTGCAGCCCGGCA |
| | | | GAAGCCTGCGGCTGAGCTGCGCAGCCTCTGGCTTCACCTTTAGCTCCTA |
| | | | CGGCATGCACTGGGTGCGGCAGGCCCCTGGCAAGGGACTGGAGTGGGT |
| | | | GGCCTTCATCAGATATGACGGCAGCAATAAGTACTATGCCGATTCCGTG |
| | | | AAGGGCAGGTTTACCATCAGCCGCGACAACTCCAAGAATACACTGTAC |
| | | | CTGCAGATGAACAGCCTGAGGGCCGAGGACACCGCCGTGTACTATTGT |
| | | | AAGACACACGGCTCCCACGATAATTGGGGCCAGGGCACCATGGTGACA |
| | | | GTGTCTAGCGGCGGCTCTGGCGGCGGCAGCATGAGCGGGCGGAGCGCC |
| | | | AACGCAGGGGGTGGCTCTGGAGGAGGCAGCGGAGGAGGCTCCGGCCA |
| | | | GTCTGTGCTGACCCAGCCACCTTCTGTGAGCGGAGCCCCCGGACAGAGG |
| | | | GTGACAATCTCCTGCTCTGGCAGCCGCTCCAACATCGGCAGCAATACCG |
| | | | TGAAGTGGTATCAGCAGCTGCCAGGCACAGCCCCCAAGCTGCTGATCTA |
| | | | CTATAATGACCAGCGGCCTTCCGGCGTGCCAGATAGATTCTCTGGCAGC |
| | | | AAGTCCGGCACCTCTGCCAGCCTGGCCATCACAGGCCTGCAGGCAGAG |
| | | | GACGAGGCAGATTACTATTGTCAGTCCTACGATAGATATACCCACCCCG |
| | | | CCCTGCTGTTTGGCACCGGCACAAAGGTGACAGTGCTG |
| 156 | 23364 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGAGGAGGCTCCGGCGGAGGAGGCTCCATGA |
| | | | GCGGGCGGAGCGCCAACGCAGGGGGCGGCGGCTCCGGCGGCGGCGGCT |
| | | | CTCAGGTGCAGCTGGTGGAGAGCGGAGGAGGAGTGGTGCAGCCCGGCA |
| | | | GAAGCCTGCGGCTGAGCTGCGCAGCCTCTGGCTTCACCTTTAGCTCCGC |
| | | | CGGCATGCACTGGGTGCGGCAGGCCCCTGGCAAGGGACTGGAGTGGGT |
| | | | GGCCTTCATCAGATATGACGGCAGCAATAAGTACTATGCCGATTCCGTG |
| | | | AAGGGCAGGTTTACCATCAGCCGCGACAACTCCAAGAATACACTGTAC |
| | | | CTGCAGATGAACAGCCTGAGGGCCGAGGACACCGCCGTGTACTATTGT |
| | | | AAGACACACGGCTCCCACGATAATTGGGGCCAGGGCACCATGGTGACA |
| | | | GTGTCTAGCGGCGGCTCTGGCGGCGGCAGCATGAGCGGGCGGAGCGCC |
| | | | AACGCAGGGGGTGGCTCTGGAGGAGGCAGCGGAGGAGGCTCCGGCCA |
| | | | GTCTGTGCTGACCCAGCCACCTTCTGTGAGCGGAGCCCCCGGACAGAGG |
| | | | GTGACAATCTCCTGCTCTGGCAGCCGCTCCAACATCGGCAGCAATACCG |
| | | | TGAAGTGGTATCAGCAGCTGCCAGGCACAGCCCCCAAGCTGCTGATCTA |
| | | | CTATAATGACCAGCGGCCTTCCGGCGTGCCAGATAGATTCTCTGGCAGC |
| | | | AAGTCCGGCACCTCTGCCAGCCTGGCCATCACAGGCCTGCAGGCAGAG |
| | | | GACGAGGCAGATTACTATTGTCAGTCCTACGATAGATATACCCACCCCG |
| | | | CCCTGCTGTTTGGCACCGGCACAAAGGTGACAGTGCTG |
| 157 | 23512 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGAGGAGGCTCTGGAGGAGGAGGCTCCGGCG |
| | | | GCGGGAGCGGCGGCGGCAGCGGGGGCGGCGGCTCTGGCGGCGGCGGC |
| | | | AGCCAGGTGCAGCTGGTGGAGAGCGGCGGCGGCGTGGTGCAGCCCGGC |
| | | | AGAAGCCTGCGGCTGAGCTGCGCAGCCTCTGGCTTCACCTTTAGCTCCT |
| | | | ACGGCATGCACTGGGTGCGGCAGGCCCCTGGCAAGGGACTGGAGTGGG |
| | | | TGGCCTTCATCAGATATGACGGCTCCAATAAGTACTATGCCGATTCTGT |
| | | | GAAGGGCAGGTTTACCATCAGCCGCGACAACTCCAAGAATACACTGTA |
| | | | CCTGCAGATGAACTCCCTGAGGGCCGAGGACACCGCCGTGTACTATTGC |
| | | | AAGACACACGGCTCTCACGATAATTGGGGCCAGGGCACCATGGTGACA |
| | | | GTGTCTTCCGGAGGAGGAGGCAGCGGCGGCGGGAGCGGCGGCGGCAGC |
| | | | GGGGGTGGAGGCTCCGGAGGAGGAGGCTCTCAGAGCGTGCTGACCCAG |
| | | | CCACCTTCCGTGTCTGGAGCCCCCGGACAGAGGGTGACAATCAGCTGTT |
| | | | CCGGCTCTCGCAGCAACATCGGCAGCAATACCGTGAAGTGGTATCAGC |
| | | | AGCTGCCAGGCACAGCCCCCAAGCTGCTGATCTACTATAATGACCAGCG |
| | | | GCCTTCCGGCGTGCCAGATAGATTCTCCGGCTCTAAGAGCGGCACCTCC |
| | | | GCCTCTCTGGCCATCACAGGCCTGCAGGCAGAGGACGAGGCAGATTAC |
| | | | TATTGTCAGTCCTACGATAGATATACCCACCCCGCCCTGCTGTTTGGCA |
| | | | CCGGCACAAAGGTGACAGTGCTG |
| 158 | 23513 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCAGGAGGAGGAGGAGGCTCTGGCGGCGGCAGCGGCGGCG |
| | | | GCAGCGGAGGAGGAGGCAGCGGAGGAGGAGGCTCCGGAGGCGGCGGC |
| | | | TCTGGCGGCGGCGGCAGCAAGATCGACGCCTGCAAGCGCGGCGATGTG |
| | | | ACAGTGAAGCCTAGCCACGTGATCCTGCTGGGCTCCACCGTGAATATCA |
| | | | CATGCTCTCTGAAGCCACGGCAGGGCTGTTTCCACTACTCCCGGAGAAA |
| | | | CAAGCTGATCCTGTATAAGTTCGACAGGCGCATCAACTTTCACCACGGC |
| | | | CACTCTCTGAATAGCCAGGTGACCGGACTGCCACTGGGCACCACACTGT |
| | | | TCGTGTGCAAGCTGGCCTGTATCAATAGCGATGAGATCCAGATCTGTGG |
| | | | AGCCGAGATCTTTGTGGGAGTGGCCCCCGAGCAGCCTCAGAACCTGTCC |
| | | | TGCATCCAGAAGGGAGAGCAGGGAACCGTGGCATGTACATGGGAGCGG |
| | | | GGCAGAGACACCCACCTGTACACCGAGTATACACTGCAGCTGAGCGGC |
| | | | CCCAAGAACCTGACATGGCAGAAGCAGTGCAAGGATATCTACTGTGAC |
| | | | TATCTGGATTTCGGCATCAACCTGACCCCAGAGTCCCCCGAGTCTAACT |
| | | | TCACCGCCAAGGTGACAGCCGTGAACTCTCTGGGCAGCTCCTCTAGCCT |
| | | | GCCCAGCACCTTCACATTTCTGGACATCGTGCGCCCTCTGCCTCCATGG |
| | | | GATATCCGGATCAAGTTTCAGAAGGCCTCCGTGTCTAGATGCACACTGT |
| | | | ACTGGAGGGACGAGGGCCTGGTGCTGCTGAACAGGCTGCGCTATAGAC |
| | | | CCAGCAATTCCAGACTGTGGAACATGGTGAATGTGACCAAGGCCAAGG |
| | | | GCAGGCACGACCTGCTGGATCTGAAGCCTTTCACAGAGTACGAGTTTCA |
| | | | GATCTCCTCTAAGCTGCACCTGTATAAGGGCTCTTGGAGCGATTGGTCC |
| | | | GAGTCTCTGAGGGCACAGACCCCTGAGGAGGAGCCA |
| 159 | 23710 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCACAGGTGTACGTGTATCCCCCTTCCCGGGACGAGCTGACCAAGA |
| | | | ACCAGGTGTCTCTGACATGCCTGGTGAAGGGCTTCTACCCCAGCGATAT |
| | | | CGCCGTGGAGTGGGAGTCCAATGGCCAGCCTGAGAACAATTATAAGAC |
| | | | CACACCACCCGTGCTGGACAGCGATGGCTCCTTCGCCCTGGTGTCCAAG |
| | | | CTGACCGTGGACAAGTCTAGGTGGCAGCAGGGCAACGTGTTTTCTTGCA |
| | | | GCGTGATGCACGAGGCCCTGCACAATCACTACACCCAGAAGTCCCTGA |
| | | | GCTTAAGCCCAGGAGGCTCCGCCGACGGAGGAATGAGCGGGCGGAGCG |
| | | | CCAACGCCGGGAGCGCAGACGGCGGCATCTGGGAGCTGAAGAAGGAC |
| | | | GTGTACGTGGTGGAGCTGGACTGGTACCCGGATGCACCAGGAGAGATG |
| | | | GTGGTGCTGACCTGCGACACACCTGAGGAGGATGGCATCACCTGGACA |
| | | | CTGGACCAGAGCTCCGAGGTGCTGGGCAGCGGCAAGACCCTGACAATC |
| | | | CAGGTGAAGGAGTTCGGCGATGCCGGACAGTACACATGTCACAAGGGC |
| | | | GGCGAGGTGCTGTCTCACAGCCTGCTGCTGCTGCACAAGAAGGAGGAT |
| | | | GGCATCTGGTCTACAGACATCCTGAAGGATCAGAAGGAGCCTAAGAAC |
| | | | AAGACCTTCCTGAGGTGCGAGGCCAAGAATTATAGCGGCAGATTCACC |
| | | | TGTTGGTGGCTGACCACAATCTCCACCGACCTGACATTTTCTGTGAAGT |
| | | | CTAGCCGGGGCTCCTCTGATCCACAGGGAGTGACATGCGGAGCCGCCA |
| | | | CCCTGTCCGCCGAGCGGGTGAGAGGCGACAACAAGGAGTACGAGTATT |
| | | | CTGTGGAGTGCCAGGAGGATAGCGCCTGTCCCGCCGCCGAGGAGTCTCT |
| | | | GCCTATCGAAGTGATGGTGGACGCCGTGCACAAGCTGAAGTACGAGAA |
| | | | TTATACAAGCTCCTTCTTTATCCGGGACATCATCAAGCCAGATCCCCCT |
| | | | AAGAACCTGCAGCTGAAGCCCCTGAAGAATAGCAGACAGGTGGAGGTG |
| | | | TCCTGGGAGTACCCTGATACCTGGTCCACACCACACTCTTATTTCAGCCT |
| | | | GACCTTTTGCGTGCAGGTGCAGGGCAAGAGCAAGAGGGAGAAGAAGG |
| | | | ACCGCGTGTTCACCGATAAGACATCCGCCACCGTGATCTGTCGGAAGAA |
| | | | CGCCAGCATCTCTGTGAGAGCCCAGGACCGGTACTATTCTAGCTCCTGG |
| | | | AGCGAGTGGGCCTCCGTGCCTTGTTCTGGAGGAGGAGGCAGCGGCGGA |
| | | | GGAGGCTCCGGAGGAGGAGGCTCTAATCTGCCAGTGGCCACCCCAGAC |
| | | | CCCGGAATGTTCCCATGCCTGCACCACTCCCAGAACCTGCTGCGGGCCG |
| | | | TGTCTAATATGCTGCAGAAGGCCAGACAGACACTGGAGTTTTACCCTTG |
| | | | TACCTCTGAGGAGATCGACCACGAGGATATCACAAAGGATAAGACCAG |
| | | | CACAGTGGAGGCCTGCCTGCCACTGGAGCTGACCAAGAACGAGAGCTG |
| | | | TCTGAACAGCCGGGAGACCAGCTTCATCACCAACGGCAGCTGCCTGGC |
| | | | CTCCAGAAAGACATCTTTTATGATGGCCCTGTGCCTGAGCAGCATCTAC |
| | | | GAGGACCTGAAGATGTATCAGGTGGAGTTCAAGACCATGAACGCCAAG |
| | | | CTGCTGATGGACCCCAAGCGGCAGATCTTTCTGGATCAGAATATGCTGG |
| | | | CCGTGATCGACGAGCTGATGCAGGCCCTGAACTTTAATAGCGAGACAG |
| | | | TGCCTCAGAAGTCCTCTCTGGAGGAGCCAGATTTCTACAAGACCAAGAT |
| | | | CAAGCTGTGCATCCTGCTGCACGCCTTCCGGATCAGAGCCGTGACCATC |
| | | | GACAGAGTGATGAGCTACCTGAACGCCAGC |
| 160 | 23711 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCACAGGTGTACGTGTATCCCCCTTCCCGGGACGAGCTGACCAAGA |
| | | | ACCAGGTGTCTCTGACATGCCTGGTGAAGGGCTTCTACCCCAGCGATAT |
| | | | CGCCGTGGAGTGGGAGTCCAATGGCCAGCCTGAGAACAATTATAAGAC |
| | | | CACACCACCCGTGCTGGACAGCGATGGCTCCTTCGCCCTGGTGTCCAAG |
| | | | CTGACCGTGGACAAGTCTAGGTGGCAGCAGGGCAACGTGTTTTCTTGCA |
| | | | GCGTGATGCACGAGGCCCTGCACAATCACTACACCCAGAAGTCCCTGA |
| | | | GCTTAAGCCCCGGCGGCTCTGCCGACGGAGGAATGAGCGGGCGGAGCG |
| | | | CCAACGCCGGGAGCGCAGACGGCGGCATCTGGGAGCTGAAGAAGGAC |
| | | | GTGTACGTGGTGGAGCTGGATTGGTACCCGGATGCCCCAGGCGAGATG |
| | | | GTGGTGCTGACCTGCGACACACCCGAGGAGGATGGCATCACCTGGACA |
| | | | CTGGATCAGAGCTCCGAGGTGCTGGGCTCCGGCAAGACCCTGACAATC |
| | | | CAGGTGAAGGAGTTCGGCGACGCCGGACAGTACACATGTCACAAGGGA |
| | | | GGAGAGGTGCTGAGCCACTCCCTGCTGCTGCTGCACAAGAAGGAGGAC |
| | | | GGCATCTGGTCCACAGACATCCTGAAGGATCAGAAGGAGCCCAAGAAC |
| | | | AAGACCTTCCTGAGGTGCGAGGCCAAGAATTATAGCGGCAGATTCACC |
| | | | TGTTGGTGGCTGACCACAATCTCCACCGATCTGACATTTTCTGTGAAGT |
| | | | CTAGCCGGGGCTCCTCTGACCCTCAGGGAGTGACATGCGGAGCCGCCA |
| | | | CCCTGTCCGCCGAGCGGGTGAGAGGCGATAACAAGGAGTACGAGTATT |
| | | | CTGTGGAGTGCCAGGAGGACAGCGCCTGTCCAGCCGCCGAGGAGAGCC |
| | | | TGCCAATCGAAGTGATGGTGGATGCCGTGCACAAGCTGAAGTACGAGA |
| | | | ATTATACAAGCTCCTTCTTTATCAGGGACATCATCAAGCCCGATCCCCC |
| | | | TAAGAACCTGCAGCTGAAGCCTCTGAAGAATAGCCGCCAGGTGGAGGT |
| | | | GTCCTGGGAGTACCCCGACACCTGGAGCACACCTCACTCCTATTTCTCT |
| | | | CTGACCTTTTGCGTGCAGGTGCAGGGCGAGAGCAAGCAGGAGAAGAAG |
| | | | GACAGGGTGTTCACCGATAAGACATCCGCCACCGTGATCTGTCGCAAG |
| | | | AACGCCTCTATCAGCGTGCGGGCACAGGACCGGTACTATTCTAGCTCCT |
| | | | GGAGCGAGTGGGCCTCCGTGCCTTGTTCTGGAGGAGGAGGCAGCGGCG |
| | | | GAGGAGGCTCCGGAGGAGGAGGCTCTAATCTGCCAGTGGCCACCCCAG |
| | | | ACCCCGGAATGTTCCCATGCCTGCACCACTCCCAGAACCTGCTGCGGGC |
| | | | CGTGTCTAATATGCTGCAGAAGGCCAGACAGACACTGGAGTTTTACCCT |
| | | | TGTACCTCTGAGGAGATCGACCACGAGGATATCACAAAGGATAAGACC |
| | | | AGCACAGTGGAGGCCTGCCTGCCACTGGAGCTGACCAAGAACGAGAGC |
| | | | TGTCTGAACAGCCGGGAGACCAGCTTCATCACCAACGGCAGCTGCCTG |
| | | | GCCTCCAGAAAGACATCTTTTATGATGGCCCTGTGCCTGAGCAGCATCT |
| | | | ACGAGGACCTGAAGATGTATCAGGTGGAGTTCAAGACCATGAACGCCA |
| | | | AGCTGCTGATGGACCCCAAGCGGCAGATCTTTCTGGATCAGAATATGCT |
| | | | GGCCGTGATCGACGAGCTGATGCAGGCCCTGAACTTTAATAGCGAGAC |
| | | | AGTGCCTCAGAAGTCCTCTCTGGAGGAGCCAGATTTCTACAAGACCAAG |
| | | | ATCAAGCTGTGCATCCTGCTGCACGCCTTCCGGATCAGAGCCGTGACCA |
| | | | TCGACAGAGTGATGAGCTACCTGAACGCCAGC |
| 161 | 24228 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCACAGGTGTACGTGTATCCCCCTTCCCGGGACGAGCTGACCAAGA |
| | | | ACCAGGTGTCTCTGACATGCCTGGTGAAGGGCTTCTACCCCAGCGATAT |
| | | | CGCCGTGGAGTGGGAGTCCAATGGCCAGCCTGAGAACAATTATAAGAC |
| | | | CACACCACCCGTGCTGGACAGCGATGGCTCCTTCGCCCTGGTGTCCAAG |
| | | | CTGACCGTGGACAAGTCTAGGTGGCAGCAGGGCAACGTGTTTTCTTGCA |
| | | | GCGTGATGCACGAGGCCCTGCACAATCACTACACCCAGAAGTCCCTGA |
| | | | GCTTAAGCCCAGGAGGCAGCGCCGATGGAGGAATCTGGGAGCTGAAGA |
| | | | AGGACGTGTACGTGGTGGAGCTGGACTGGTACCCGGATGCCCCAGGCG |
| | | | AGATGGTGGTGCTGACCTGCGACACACCTGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACATGTCACA |
| | | | AGGGAGGAGAGGTGCTGAGCCACTCCCTGCTGCTGCTGCACAAGAAGG |
| | | | AGGACGGCATCTGGAGCACAGACATCCTGAAGGATCAGAAGGAGCCAA |
| | | | AGAACAAGACCTTCCTGCGGTGCGAGGCCAAGAATTATAGCGGCAGAT |
| | | | TCACCTGTTGGTGGCTGACCACAATCAGCACCGATCTGACATTTTCCGT |
| | | | GAAGTCTAGCAGGGGCTCCTCTGACCCCCAGGGAGTGACATGCGGAGC |
| | | | CGCCACCCTGAGCGCCGAGCGGGTGAGAGGCGATAACAAGGAGTACGA |
| | | | GTATTCCGTGGAGTGCCAGGAGGACTCTGCCTGTCCAGCCGCCGAGGA |
| | | | GTCCCTGCCAATCGAAGTGATGGTGGATGCCGTGCACAAGCTGAAGTA |
| | | | CGAGAATTATACAAGCTCCTTCTTTATCAGGGACATCATCAAGCCCGAT |
| | | | CCCCCTAAGAACCTGCAGCTGAAGCCCCTGAAGAACAGCAGACAGGTG |
| | | | GAGGTGAGCTGGGAGTACCCCGACACCTGGAGCACACCTCACTCCTATT |
| | | | TCTCTCTGACCTTTTGCGTGCAGGTGCAGGGCAAGTCTAAGAGGGAGAA |
| | | | GAAGGACCGCGTGTTCACCGATAAGACAAGCGCCACCGTGATCTGTAG |
| | | | AAAGAACGCCTCTATCAGCGTGCGGGCACAGGACCGGTACTACAGCTC |
| | | | CTCTTGGAGCGAGTGGGCCTCCGTGCCCTGCTCTGGCGGCGGCGGCTCT |
| | | | GGAGGAGGAGGCAGCGGCGGAGGAGGCTCCAACCTGCCTGTGGCCACC |
| | | | CCCGATCCTGGCATGTTCCCATGCCTGCACCACTCTCAGAACCTGCTGA |
| | | | GGGCCGTGTCCAATATGCTGCAGAAGGCCCGCCAGACACTGGAGTTTTA |
| | | | TCCCTGTACCAGCGAGGAGATCGACCACGAGGATATCACAAAGGACAA |
| | | | GACCTCCACAGTGGAGGCCTGCCTGCCTCTGGAGCTGACCAAGAACGA |
| | | | GAGCTGTCTGAACAGCCGGGAGACCAGCTTCATCACCAATGGCAGCTG |
| | | | CCTGGCCTCCAGAAAGACATCTTTTATGATGGCCCTGTGCCTGAGCTCC |
| | | | ATCTACGAGGATCTGAAGATGTATCAGGTGGAGTTCAAGACCATGAAC |
| | | | GCCAAGCTGCTGATGGACCCTAAGCGGCAGATCTTTCTGGATCAGAATA |
| | | | TGCTGGCCGTGATCGACGAGCTGATGCAGGCCCTGAACTTCAATAGCGA |
| | | | GACAGTGCCCCAGAAGTCTAGCCTGGAGGAGCCTGATTTCTACAAGAC |
| | | | CAAGATCAAGCTGTGCATCCTGCTGCACGCCTTTCGGATCAGAGCCGTG |
| | | | ACCATCGACAGAGTGATGTCTTATCTGAACGCCAGCGGCGGCGGAGGC |
| | | | TCTATGAGCGGGCGGAGCGCCAACGCAGGGGGAGGAGGCTCCGGAGG |
| | | | AGGAGGCTCTGGCGGCGGCGGCAGCGGAGGCGGCGGCTCCGAGATCGT |
| | | | GATGACACAGAGCCCTGCCACCCTGTCCGTGTCTCCAGGAGAGAGGGC |
| | | | CACACTGTCCTGTAGAGCCAGCCAGTCCATCTCTATCAACCTGCACTGG |
| | | | TATCAGCAGAAGCCAGGCCAGGCCCCCAGGCTGCTGATCTATTTCGCCA |
| | | | GCCAGAGCATTTCTGGCATCCCTGCACGCTTCAGCGGCTCCGGCTCTGG |
| | | | CACCGAGTTTACCCTGACAATCTCCTCTCTGCAGAGCGAGGATTTTGCC |
| | | | GTGTACTATTGCCAGCAGAGCAATTCCTTCCCACTGACATTTGGCGGCG |
| | | | GCACCAAGGTGGAGATCAAGGGAGGCAGCGGCGGCGGCTCCGGCGGC |
| | | | GGCTCTGGGGGAGGCAGCGGAGGAGGCTCCGGACAGGTGCAGCTGGTG |
| | | | CAGTCCGGAGCCGAGGTGAAGAAGCCAGGGGCCAGCGTGAAGGTGAG |
| | | | CTGTAAGGCCTCCGGCTACACCTTCACAGACTACTATCTGCACTGGGTG |
| | | | AGGCAGGCCCCCGGACAGGGACTGGAGTGGATGGGCTGGATCGACCCA |
| | | | GAGAACGGCGATACAGAGTACGCCCCCAAGTTTCAGGGCCGCGTGACC |
| | | | ATGACCACAGATACCTCTACAAGCACCGCCTATATGGAGCTGAGGTCCC |
| | | | TGCGCTCTGACGATACCGCCGTGTACTATTGTAACGCCAATAAGGAGCT |
| | | | GAGGTACTTTGACGTGTGGGGCCAGGGCACAATGGTGACCGTGAGCTC |
| | | | C |
| 162 | 24229 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCAGGAGGAGGAGGAGGCTCTGGAGGAGGAGGCTCTATGA |
| | | | GCGGGCGGAGCGCCAACGCAGGGGGAGGAGGCAGCGGGGGAGGAGGC |
| | | | TCCCAGGTGCAGCTGGTGGAGAGCGGCGGCGGCGTGGTGCAGCCCGGC |
| | | | AGAAGCCTGCGGCTGAGCTGCGCAGCCTCCGGCTTCACCTTTAGCTCCT |
| | | | ACGGCATGCACTGGGTGCGGCAGGCCCCTGGCAAGGGACTGGAGTGGG |
| | | | TGGCCTTCATCAGATATGACGGCTCCAATAAGTACTATGCCGATTCTGT |
| | | | GAAGGGCCGGTTTACAATCTCTAGAGACAACAGCAAGAATACCCTGTA |
| | | | CCTGCAGATGAACAGCCTGCGGGCCGAGGACACAGCCGTGTACTATTG |
| | | | CAAGACCCACGGCTCCCACGATAATTGGGGCCAGGGCACAATGGTGAC |
| | | | CGTGTCTTCCGGCGGCGGCGGCTCCATGAGCGGGCGGAGCGCCAACGC |
| | | | AGGGGGTGGAGGCTCCGGAGGAGGAGGCAGCCAGTCCGTGCTGACACA |
| | | | GCCACCTTCTGTGAGCGGAGCCCCCGGACAGAGGGTGACCATCTCCTGT |
| | | | TCTGGCAGCCGCTCCAACATCGGCAGCAATACAGTGAAGTGGTATCAG |
| | | | CAGCTGCCAGGCACCGCCCCCAAGCTGCTGATCTACTATAATGACCAGC |
| | | | GGCCTTCCGGCGTGCCAGATAGATTCTCTGGCAGCAAGTCCGGCACATC |
| | | | TGCCAGCCTGGCCATCACCGGCCTGCAGGCAGAGGACGAGGCCGATTA |
| | | | CTATTGCCAGAGCTACGATAGGTATACACACCCTGCCCTGCTGTTTGGC |
| | | | ACCGGCACAAAGGTGACCGTGCTGGGCGGCGGCGGCTCTGGCGGCGGC |
| | | | GGCAGCGGCGGAGGAGGCTCCATGAGCGGGCGGAGCGCCAACGCAGG |
| | | | GGGCGGCGGCTCCGGAGGAGGGGGCTCTGGAGGCGGCGGCAGCGAGA |
| | | | TCGTGATGACACAGTCCCCAGCCACCCTGAGCGTGTCCCCAGGAGAGA |
| | | | GGGCCACACTGTCTTGTCGCGCCTCTCAGAGCATCTCCATCAATCTGCA |
| | | | CTGGTATCAGCAGAAGCCAGGCCAGGCCCCCCGGCTGCTGATCTATTTC |
| | | | GCCTCTCAGTCCATTTCCGGCATCCCTGCACGCTTCTCTGGCAGCGGCTC |
| | | | CGGCACCGAGTTTACCCTGACAATCTCCTCTCTGCAGAGCGAGGACTTT |
| | | | GCCGTGTACTATTGCCAGCAGTCTAACAGCTTCCCACTGACATTTGGCG |
| | | | GCGGCACCAAGGTGGAGATCAAGGGCGGCTCCGGCGGCGGCTCTGGGG |
| | | | GCGGCAGCGGAGGAGGCTCCGGAGGAGGCTCTGGACAGGTGCAGCTGG |
| | | | TGCAGTCCGGAGCCGAGGTGAAGAAGCCAGGGGCCAGCGTGAAGGTGT |
| | | | CCTGTAAGGCCTCTGGCTACACCTTCACAGATTACTATCTGCATTGGGT |
| | | | GCGGCAGGCCCCCGGACAGGGACTGGAGTGGATGGGCTGGATCGACCC |
| | | | TGAGAATGGCGATACAGAGTACGCCCCAAAGTTTCAGGGCAGAGTGAC |
| | | | CATGACCACAGACACCTCCACATCTACCGCCTATATGGAGCTGAGGAGC |
| | | | CTGCGCTCCGACGATACCGCCGTGTACTATTGTAACGCCAATAAGGAGC |
| | | | TGCGGTATTTCGACGTGTGGGGACAGGGCACAATGGTCACCGTGAGCTC |
| | | | C |
| 163 | 24230 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCAGGAGGAGGAGGAGGCTCTGGAGGAGGAGGCTCTATGA |
| | | | GCGGGCGGAGCGCCAACGCAGGGGGAGGAGGCAGCGGGGGAGGAGGC |
| | | | TCCCAGGTGCAGCTGGTGGAGAGCGGCGGCGGCGTGGTGCAGCCCGGC |
| | | | AGAAGCCTGCGGCTGAGCTGTGCAGCCTCCGGCTTCACCTTTAGCTCCT |
| | | | ACGGCATGCACTGGGTGCGGCAGGCCCCTGGCAAGGGACTGGAGTGGG |
| | | | TGGCCTTCATCAGATATGACGGCTCCAATAAGTACTATGCCGATTCTGT |
| | | | GAAGGGCCGGTTTACAATCTCTAGAGACAACAGCAAGAATACCCTGTA |
| | | | CCTGCAGATGAACAGCCTGCGGGCCGAGGACACAGCCGTGTACTATTG |
| | | | CAAGACCCACGGCTCCCACGATAATTGGGGCCAGGGCACAATGGTGAC |
| | | | CGTGTCTTCCGGCGGCGGCGGCTCCATGAGCGGGCGGAGCGCCAACGC |
| | | | AGGGGGTGGAGGCTCCGGAGGAGGAGGCAGCCAGTCCGTGCTGACACA |
| | | | GCCACCTTCTGTGAGCGGAGCCCCCGGACAGAGGGTGACCATCTCCTGT |
| | | | TCTGGCAGCCGCTCCAACATCGGCAGCAATACAGTGAAGTGGTATCAG |
| | | | CAGCTGCCAGGCACCGCCCCCAAGCTGCTGATCTACTATAATGACCAGC |
| | | | GGCCTTCCGGCGTGCCAGATAGATTCTCTGGCAGCAAGTCCGGCACATC |
| | | | TGCCAGCCTGGCCATCACCGGCCTGCAGGCAGAGGACGAGGCCGATTA |
| | | | CTATTGTCAGTCCTACGACAGGTATACACACCCTGCCCTGCTGTTTGGC |
| | | | ACCGGCACAAAGGTGACCGTGCTGGGCGGCGGCGGCTCTGGCGGCGGC |
| | | | GGCAGCGGCGGAGGAGGCTCCATGAGCGGGCGGAGCGCCAACGCAGG |
| | | | GGGCGGCGGCTCCGGAGGAGGGGGCTCTGGAGGCGGCGGCAGCCAGGT |
| | | | GCAGCTGGTGCAGTCCGGAGCCGAGGTGAAGAAGCCAGGGGCCAGCGT |
| | | | GAAGGTGTCTTGCAAGGCCAGCGGCTACACCTTCACAGATTACTATCTG |
| | | | CATTGGGTGCGGCAGGCCCCCGGACAGGGACTGGAGTGGATGGGCTGG |
| | | | ATCGACCCTGAGAATGGCGATACAGAGTACGCCCCAAAGTTTCAGGGC |
| | | | AGAGTGACCATGACCACAGACACCAGCACATCCACCGCCTATATGGAG |
| | | | CTGAGGAGCCTGCGCTCCGACGATACCGCCGTGTACTATTGCAACGCCA |
| | | | ATAAGGAGCTGCGGTATTTCGACGTGTGGGGACAGGGCACAATGGTCA |
| | | | CCGTGTCCTCTGGCGGCTCCGGCGGCGGCTCTGGGGGCGGCAGCGGAG |
| | | | GAGGCTCCGGAGGAGGCTCTGGCGAGATCGTGATGACACAGTCCCCAG |
| | | | CCACCCTGTCTGTGAGCCCAGGAGAGAGGGCCACACTGTCTTGTCGCGC |
| | | | CTCCCAGTCTATCAGCATCAACCTGCACTGGTATCAGCAGAAGCCAGGC |
| | | | CAGGCCCCCCGGCTGCTGATCTATTTCGCCTCCCAGTCCATTAGCGGCA |
| | | | TCCCTGCACGCTTCTCCGGCTCTGGCAGCGGCACCGAGTTTACACTGAC |
| | | | CATCAGCTCCCTGCAGAGCGAGGATTTTGCCGTGTACTATTGCCAGCAG |
| | | | TCCAATTCTTTCCCACTGACATTTGGCGGCGGCACCAAGGTGGAGATCA |
| | | | AG |
| 164 | 24231 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCACAGGTGTACGTGTATCCCCCTTCCCGGGACGAGCTGACCAAGA |
| | | | ACCAGGTGTCTCTGACATGCCTGGTGAAGGGCTTCTACCCCAGCGATAT |
| | | | CGCCGTGGAGTGGGAGTCCAATGGCCAGCCTGAGAACAATTATAAGAC |
| | | | CACACCACCCGTGCTGGACAGCGATGGCTCCTTCGCCCTGGTGTCCAAG |
| | | | CTGACCGTGGACAAGTCTAGGTGGCAGCAGGGCAACGTGTTTTCTTGCA |
| | | | GCGTGATGCACGAGGCCCTGCACAATCACTACACCCAGAAGTCCCTGA |
| | | | GCTTAAGCCCAGGAGGCAGCGCCGATGGAGGAATCTGGGAGCTGAAGA |
| | | | AGGACGTGTACGTGGTGGAGCTGGACTGGTACCCGGATGCCCCAGGCG |
| | | | AGATGGTGGTGCTGACCTGCGACACACCTGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACATGTCACA |
| | | | AGGGAGGAGAGGTGCTGAGCCACTCCCTGCTGCTGCTGCACAAGAAGG |
| | | | AGGACGGCATCTGGAGCACAGACATCCTGAAGGATCAGAAGGAGCCAA |
| | | | AGAACAAGACCTTCCTGCGGTGCGAGGCCAAGAATTATAGCGGCAGAT |
| | | | TCACCTGTTGGTGGCTGACCACAATCAGCACCGATCTGACATTTTCCGT |
| | | | GAAGTCTAGCAGGGGCTCCTCTGACCCCCAGGGAGTGACATGCGGAGC |
| | | | CGCCACCCTGAGCGCCGAGCGGGTGAGAGGCGATAACAAGGAGTACGA |
| | | | GTATTCCGTGGAGTGCCAGGAGGACTCTGCCTGTCCAGCCGCCGAGGA |
| | | | GTCCCTGCCAATCGAAGTGATGGTGGATGCCGTGCACAAGCTGAAGTA |
| | | | CGAGAATTATACAAGCTCCTTCTTTATCAGGGACATCATCAAGCCCGAT |
| | | | CCCCCTAAGAACCTGCAGCTGAAGCCCCTGAAGAACAGCAGACAGGTG |
| | | | GAGGTGAGCTGGGAGTACCCCGACACCTGGAGCACACCTCACTCCTATT |
| | | | TCTCTCTGACCTTTTGCGTGCAGGTGCAGGGCAAGTCTAAGAGGGAGAA |
| | | | GAAGGACCGCGTGTTCACCGATAAGACAAGCGCCACCGTGATCTGTAG |
| | | | AAAGAACGCCTCTATCAGCGTGCGGGCACAGGACCGGTACTACAGCTC |
| | | | CTCTTGGAGCGAGTGGGCCTCCGTGCCCTGCTCTGGAGGAGGAGGCAG |
| | | | CGGCGGAGGAGGCTCCGGAGGCGGCGGCTCTAATCTGCCCGTGGCCAC |
| | | | CCCAGATCCCGGAATGTTCCCTTGCCTGCACCACTCCCAGAACCTGCTG |
| | | | CGCGCCGTGTCTAATATGCTGCAGAAGGCCCGGCAGACCCTGGAGTTTT |
| | | | ACCCCTGTACATCTGAGGAGATCGACCACGAGGATATCACCAAGGACA |
| | | | AGACCAGCACAGTGGAGGCCTGCCTGCCTCTGGAGCTGACAAAGAACG |
| | | | AGAGCTGTCTGAACAGCCGGGAGACCAGCTTCATCACAAACGGCAGCT |
| | | | GCCTGGCCTCCCGGAAGACCTCTTTTATGATGGCCCTGTGCCTGAGCTC |
| | | | CATCTACGAGGATCTGAAGATGTATCAGGTGGAGTTCAAGACAATGAA |
| | | | CGCCAAGCTGCTGATGGACCCTAAGAGACAGATCTTTCTGGATCAGAAC |
| | | | ATGCTGGCCGTGATCGACGAGCTGATGCAGGCCCTGAACTTCAATTCCG |
| | | | AGACCGTGCCTCAGAAGTCTAGCCTGGAGGAGCCAGATTTCTACAAGA |
| | | | CAAAGATCAAGCTGTGCATCCTGCTGCACGCCTTTCGGATCAGAGCCGT |
| | | | GACCATCGACAGAGTGATGAGCTACCTGAACGCCAGCGGCGGCGGCGG |
| | | | CTCCATGAGCGGGCGGAGCGCCAACGCAGGGGGAGGCGGCTCTGGCGG |
| | | | CGGCGGCAGCGGCGGCGGGGGCTCCGGAGGAGGAGGCTCTGGAGGCG |
| | | | GCGGCAGCAAGATCGACGCCTGTAAGCGCGGCGATGTGACCGTGAAGC |
| | | | CTTCCCACGTGATCCTGCTGGGCTCTACCGTGAATATCACATGCAGCCT |
| | | | GAAGCCACGGCAGGGCTGTTTTCACTACTCCCGGAGAAACAAGCTGAT |
| | | | CCTGTATAAGTTCGATAGGCGCATCAACTTTCACCACGGCCACTCTCTG |
| | | | AATAGCCAGGTGACAGGCCTGCCTCTGGGCACCACACTGTTCGTGTGCA |
| | | | AGCTGGCCTGTATCAATTCCGACGAGATCCAGATCTGCGGAGCCGAGAT |
| | | | CTTTGTGGGCGTGGCCCCTGAGCAGCCACAGAACCTGAGCTGCATCCAG |
| | | | AAGGGAGAGCAGGGCACCGTGGCATGTACATGGGAGAGGGGCCGCGA |
| | | | TACCCACCTGTACACCGAGTATACACTGCAGCTGAGCGGCCCAAAGAA |
| | | | CCTGACATGGCAGAAGCAGTGCAAGGACATCTACTGTGACTATCTGGAT |
| | | | TTCGGCATCAACCTGACCCCCGAGTCCCCTGAGTCTAACTTCACCGCCA |
| | | | AGGTGACAGCCGTGAACAGCCTGGGCTCCTCTAGCTCCCTGCCATCCAC |
| | | | CTTCACATTTCTGGATATCGTGAGACCCCTGCCCCCTTGGGACATCAGG |
| | | | ATCAAGTTCCAGAAGGCCAGCGTGTCCCGCTGTACACTGTACTGGCGGG |
| | | | ATGAGGGCCTGGTGCTGCTGAACCGGCTGAGATATAGGCCATCTAATA |
| | | | GCAGACTGTGGAACATGGTGAATGTGACCAAGGCCAAGGGCAGGCACG |
| | | | ACCTGCTGGATCTGAAGCCCTTCACAGAGTACGAGTTTCAGATCTCTAG |
| | | | CAAGCTGCACCTGTATAAGGGCTCCTGGTCTGACTGGAGCGAGTCCCTG |
| | | | AGGGCACAGACCCCAGAGGAGGAGCCA |
| 165 | 24232 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCAGGAGGAGGAGGAGGCTCTGGAGGAGGAGGCAGCGGA |
| | | | GGCGGCGGCTCCATGAGCGGGCGGAGCGCCAACGCAGGGGGCGGCGG |
| | | | CTCCGGCGGCGGCGGCTCTGGCGGCGGCGGCAGCTGCCGCACCTCCGA |
| | | | GTGCTGTTTCCAGGACCCCCCTTACCCTGATGCAGACAGCGGCTCCGCC |
| | | | TCTGGACCAAGAGATCTGAGGTGCTATCGCATCAGCTCCGACAGGTACG |
| | | | AGTGTAGCTGGCAGTATGAGGGACCTACCGCCGGGGTGAGCCACTTTCT |
| | | | GCGGTGCTGTCTGTCTAGCGGCAGATGCTGTTACTTCGCCGCCGGGAGC |
| | | | GCCACAAGGCTGCAGTTTAGCGACCAGGCCGGGGTGAGCGTGCTGTAT |
| | | | ACCGTGACACTGTGGGTGGAGTCCTGGGCCAGAAACCAGACCGAGAAG |
| | | | TCTCCTGAGGTGACACTGCAGCTGTACAATTCTGTGAAGTATGAGCCAC |
| | | | CCCTGGGCGATATCAAGGTGAGCAAGCTGGCCGGGCAGCTGAGGATGG |
| | | | AGTGGGAGACCCCAGACAATCAAGTGGGAGCCGAGGTGCAGTTCCGCC |
| | | | ACAGGACACCATCCTCTCCATGGAAGCTGGGCGATTGCGGACCACAGG |
| | | | ACGATGACACAGAGTCCTGCCTGTGCCCTCTGGAGATGAACGTGGCCCA |
| | | | GGAGTTTCAGCTGCGGAGAAGGCAGCTGGGCTCTCAGGGCAGCTCCTG |
| | | | GAGCAAGTGGTCTAGCCCCGTGTGCGTGCCTCCAGAGAATCCCCCTCAG |
| | | | CCCCAG |
| 166 | 24233 | Full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGCGGCGGCTCCATGAGCGGGCGGAGCGCCA |
| | | | ACGCAGGGGGAGGAGGCAGCGGCGGAGGAGGCTCCGGCGGCGGCGGC |
| | | | TCTGGAGGAGGAGGCAGCAAGATCGATGCATGCAAGAGGGGCGACGTG |
| | | | ACCGTGAAGCCTAGCCACGTGATCCTGCTGGGCTCCACCGTGAACATCA |
| | | | CATGCTCTCTGAAGCCACGCCAGGGCTGTTTCCACTACTCCCGGAGAAA |
| | | | TAAGCTGATCCTGTATAAGTTCGATAGGCGCATCAACTTTCACCACGGC |
| | | | CACTCTCTGAATAGCCAGGTGACAGGCCTGCCCCTGGGCACCACACTGT |
| | | | TCGTGTGCAAGCTGGCCTGTATCAACAGCGACGAGATCCAGATCTGCGG |
| | | | AGCCGAGATCTTTGTGGGCGTGGCCCCTGGAGGAGGAGGCTCCGGAGG |
| | | | AGGCGGCTCTGGCGGCGGCGGCAGCATGAGCGGGCGGAGCGCCAACGC |
| | | | AGGGGGTGGCGGCAGCGGCGGCGGCGGCTCCGGAGGGGGCGGCTCCTG |
| | | | TCGCACCTCTGAGTGCTGTTTCCAGGACCCCCCTTACCCTGATGCAGAC |
| | | | TCTGGCAGCGCCTCCGGACCAAGAGATCTGAGGTGCTATCGCATCAGCT |
| | | | CCGACAGATACGAGTGTTCTTGGCAGTATGAGGGACCAACCGCCGGGG |
| | | | TGAGCCACTTTCTGCGGTGCTGTCTGTCTAGCGGCAGATGCTGTTACTTC |
| | | | GCCGCCGGGAGCGCCACAAGGCTGCAGTTTTCTGACCAGGCCGGGGTG |
| | | | AGCGTGCTGTATACCGTGACACTGTGGGTGGAGAGCTGGGCCAGAAAC |
| | | | CAGACCGAGAAGTCCCCAGAGGTGACACTGCAGCTGTACAATTCCGTG |
| | | | AAGTATGAGCCACCCCTGGGCGATATCAAGGTGTCTAAGCTGGCCGGG |
| | | | CAGCTGAGGATGGAGTGGGAGACCCCCGACAATCAAGTGGGAGCCGAG |
| | | | GTGCAGTTCCGCCACAGGACACCATCCTCTCCATGGAAGCTGGGCGATT |
| | | | GCGGACCACAGGACGATGACACCGAGTCCTGCCTGTGCCCTCTGGAGA |
| | | | TGAACGTGGCCCAGGAGTTTCAGCTGCGGAGAAGGCAGCTGGGCTCCC |
| | | | AGGGCAGCTCCTGGTCTAAGTGGTCTAGCCCCGTGTGCGTGCCTCCAGA |
| | | | GAATCCCCCTCAGCCCCAG |
| 167 | 24235 | Full nt | AAGATCGACGCATGCAAGAGGGGCGATGTGACAGTGAAGCCTTCTCAC |
| | | | GTGATCCTGCTGGGCAGCACCGTGAACATCACATGCTCCCTGAAGCCCA |
| | | | GACAGGGCTGTTTTCACTACTCCCGGAGAAATAAGCTGATCCTGTATAA |
| | | | GTTCGATAGGCGCATCAACTTTCACCACGGCCACTCTCTGAATAGCCAG |
| | | | GTGACCGGACTGCCTCTGGGCACCACACTGTTCGTGTGCAAGCTGGCCT |
| | | | GTATCAACTCTGACGAGATCCAGATCTGTGGAGCCGAGATCTTTGTGGG |
| | | | CGTGGCCCCAGGAGGAGGAGGCAGCGGAGGAGGCGGCAGCATGAGCG |
| | | | GCAGAAGCGCCAACGCCGGAGGAGGAGGCAGCAGAAATCTGCCAGTG |
| | | | GCCACACCAGACCCCGGAATGTTCCCTTGCCTGCACCACTCCCAGAACC |
| | | | TGCTGAGGGCCGTGTCTAATATGCTGCAGAAGGCCCGCCAGACCCTGG |
| | | | AGTTTTACCCATGTACAAGCGAGGAGATCGACCACGAGGATATCACCA |
| | | | AGGATAAGACCTCCACAGTGGAGGCATGCCTGCCACTGGAGCTGACAA |
| | | | AGAACGAGTCCTGTCTGAACAGCCGGGAGACCAGCTTCATCACAAACG |
| | | | GCTCCTGCCTGGCCTCTAGAAAGACCAGCTTTATGATGGCCCTGTGCCT |
| | | | GAGCTCCATCTACGAGGACCTGAAGATGTATCAGGTGGAGTTCAAGAC |
| | | | AATGAACGCCAAGCTGCTGATGGACCCCAAGCGGCAGATCTTTCTGGAT |
| | | | CAGAATATGCTGGCCGTGATCGACGAGCTGATGCAGGCCCTGAACTTCA |
| | | | ATAGCGAGACCGTGCCTCAGAAGTCTAGCCTGGAGGAGCCAGATTTCT |
| | | | ACAAGACAAAGATCAAGCTGTGCATCCTGCTGCACGCCTTTCGGATCAG |
| | | | AGCCGTGACCATCGACAGAGTGATGTCCTACCTGAACGCCAGCGGCGG |
| | | | CGGCGGCAGCGGCGGAGGCGGCTCCGAGCCTAAGTCCTCTGATAAGAC |
| | | | CCACACATGCCCCCCTTGTCCGGCGCCAGAGGCTGCAGGAGGACCAAG |
| | | | CGTGTTCCTGTTTCCACCCAAGCCTAAAGACACACTGATGATTTCCCGA |
| | | | ACCCCCGAAGTCACATGCGTGGTCGTGTCTGTGAGTCACGAGGACCCTG |
| | | | AAGTCAAGTTCAACTGGTACGTGGATGGCGTCGAGGTGCATAATGCCA |
| | | | AGACTAAACCTAGGGAGGAACAGTACAACTCAACCTATCGCGTCGTGA |
| | | | GCGTCCTGACAGTGCTGCACCAGGATTGGCTGAACGGCAAAGAATATA |
| | | | AGTGCAAAGTGAGCAATAAGGCCCTGCCCGCTCCTATCGAGAAAACCA |
| | | | TTTCCAAGGCTAAAGGGCAGCCTCGCGAACCACAGGTCTACGTGTATCC |
| | | | TCCAAGCCGGGACGAGCTGACAAAGAACCAGGTCTCCCTGACTTGTCTG |
| | | | GTGAAAGGGTTTTACCCTAGTGATATCGCTGTGGAGTGGGAATCAAATG |
| | | | GACAGCCAGAGAACAATTATAAGACTACCCCCCCTGTGCTGGACAGTG |
| | | | ATGGGTCATTCGCACTGGTCTCCAAGCTGACAGTGGACAAATCTCGGTG |
| | | | GCAGCAGGGAAATGTCTTTTCATGTAGCGTGATGCATGAAGCACTGCAC |
| | | | AACCATTACACCCAGAAGTCACTGTCACTGTCACCAGGA |
| 168 | 24236 | Full nt | TGCAGGACAAGCGAGTGCTGTTTTCAGGACCCCCCTTACCCAGATGCAG |
| | | | ACAGCGGCTCCGCCTCTGGACCCCGGGACCTGCGGTGCTATAGAATCAG |
| | | | CTCCGACCGCTACGAGTGTTCTTGGCAGTATGAGGGACCTACCGCCGGG |
| | | | GTGAGCCACTTCCTGAGGTGCTGTCTGTCTAGCGGCAGATGCTGTTACT |
| | | | TCGCCGCCGGGAGCGCCACAAGGCTGCAGTTTTCTGACCAGGCCGGGG |
| | | | TGAGCGTGCTGTATACCGTGACACTGTGGGTGGAGAGCTGGGCCAGAA |
| | | | ACCAGACCGAGAAGTCCCCAGAGGTGACACTGCAGCTGTACAATTCCG |
| | | | TGAAGTATGAGCCACCCCTGGGCGATATCAAGGTGTCTAAGCTGGCCG |
| | | | GGCAGCTGAGGATGGAGTGGGAGACCCCCGACAATCAAGTGGGAGCCG |
| | | | AGGTGCAGTTCCGCCACAGGACACCTTCCTCTCCATGGAAGCTGGGCGA |
| | | | TTGCGGCCCACAGGACGATGACACCGAGAGCTGCCTGTGCCCCCTGGA |
| | | | GATGAACGTGGCCCAGGAGTTTCAGCTGCGGAGAAGGCAGCTGGGCTC |
| | | | CCAGGGCAGCTCCTGGTCTAAGTGGTCTAGCCCCGTGTGCGTGCCTCCA |
| | | | GAGAATCCCCCTCAGCCACAGGGCGGCGGCGGCTCTGGAGGAGGAGGC |
| | | | AGCGGCGGAGGAGGCTCCGGAGGCGGCGGCAGCATGTCCGGCAGGTCC |
| | | | GCCAACGCCGAGCCCAAGTCCTCTGACAAGACCCACACATGCCCACCCT |
| | | | GTCCGGCGCCAGAGGCCGCCGGAGGACCAAGCGTGTTCCTGTTTCCACC |
| | | | CAAGCCCAAGGACACCCTGATGATCTCCCGGACCCCAGAGGTGACATG |
| | | | CGTGGTGGTGAGCGTGTCCCACGAGGACCCCGAGGTGAAGTTTAACTG |
| | | | GTACGTGGATGGCGTGGAGGTGCACAATGCCAAGACAAAGCCCCGGGA |
| | | | GGAGCAGTACAATTCTACCTATAGAGTGGTGAGCGTGCTGACAGTGCTG |
| | | | CACCAGGATTGGCTGAACGGCAAGGAGTATAAGTGTAAGGTGAGCAAT |
| | | | AAGGCCCTGCCAGCCCCCATCGAGAAGACCATCTCCAAGGCCAAGGGC |
| | | | CAGCCTCGCGAACCACAGGTCTACGTGCTGCCCCCTAGCCGCGACGAAC |
| | | | TGACTAAAAATCAGGTCTCTCTGCTGTGTCTGGTCAAAGGATTCTACCC |
| | | | TTCCGACATCGCCGTGGAGTGGGAAAGTAACGGCCAGCCCGAGAACAA |
| | | | TTACCTGACCTGGCCCCCTGTGCTGGACTCTGATGGGAGTTTCTTTCTGT |
| | | | ATTCAAAGCTGACAGTCGATAAAAGCCGGTGGCAGCAGGGCAATGTGT |
| | | | TCAGCTGCTCCGTCATGCACGAAGCACTGCACAACCATTACACTCAGAA |
| | | | GTCCCTGTCCCTGTCACCTGGC |
| 169 | 24246 | Full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCACAGGTGTACGTGTATCCCCCTTCCCGGGACGAGCTGACCAAGA |
| | | | ACCAGGTGTCTCTGACATGCCTGGTGAAGGGCTTCTACCCCAGCGATAT |
| | | | CGCCGTGGAGTGGGAGTCCAATGGCCAGCCTGAGAACAATTATAAGAC |
| | | | CACACCACCCGTGCTGGACAGCGATGGCTCCTTCGCCCTGGTGTCCAAG |
| | | | CTGACCGTGGACAAGTCTAGGTGGCAGCAGGGCAACGTGTTTTCTTGCA |
| | | | GCGTGATGCACGAGGCCCTGCACAATCACTACACCCAGAAGTCCCTGA |
| | | | GCTTAAGCCCTGGCGGCGGCGGCGGCTCCATGAGCGGGCGGAGCGCCA |
| | | | ACGCAGGGGGAGGAGGCTCCGGCGGAGGAGGCTCTGGCGGCGGCGGC |
| | | | AGCGGAGGAGGCGGCTCCAAGATCGACGCCTGCAAGCGGGGCGATGTG |
| | | | ACCGTGAAGCCCTCCCACGTGATCCTGCTGGGCTCTACCGTGAACATCA |
| | | | CATGCAGCCTGAAGCCTAGACAGGGCTGTTTCCACTACAGCCGGAGAA |
| | | | ATAAGCTGATCCTGTATAAGTTCGATAGGCGCATCAACTTTCACCACGG |
| | | | CCACTCTCTGAATAGCCAGGTGACAGGCCTGCCTCTGGGCACCACACTG |
| | | | TTCGTGTGCAAGCTGGCCTGTATCAATTCCGACGAGATCCAGATCTGTG |
| | | | GAGCCGAGATCTTTGTGGGCGTGGCCCCAGGAGGAGGAGGCTCTGGAG |
| | | | GAGGCGGCAGCATGAGCGGGCGGAGCGCCAACGCAGGGGGTGGAGGC |
| | | | TCTCGCAATCTGCCTGTGGCCACCCCCGATCCTGGCATGTTCCCATGCCT |
| | | | GCACCACAGCCAGAACCTGCTGCGGGCCGTGTCCAATATGCTGCAGAA |
| | | | GGCCAGACAGACCCTGGAGTTTTACCCATGTACATCTGAGGAGATCGAC |
| | | | CACGAGGATATCACCAAGGACAAGACCAGCACAGTGGAGGCATGCCTG |
| | | | CCACTGGAGCTGACAAAGAACGAGTCCTGTCTGAACAGCCGGGAGACC |
| | | | AGCTTCATCACAAACGGCTCCTGCCTGGCCTCTCGCAAGACCAGCTTTA |
| | | | TGATGGCCCTGTGCCTGAGCTCCATCTACGAGGATCTGAAGATGTATCA |
| | | | GGTGGAGTTCAAGACAATGAACGCCAAGCTGCTGATGGACCCTAAGAG |
| | | | GCAGATCTTTCTGGATCAGAATATGCTGGCCGTGATCGACGAGCTGATG |
| | | | CAGGCCCTGAACTTTAATTCCGAGACCGTGCCACAGAAGTCTAGCCTGG |
| | | | AGGAGCCCGATTTCTACAAGACAAAGATCAAGCTGTGCATCCTGCTGCA |
| | | | CGCCTTTCGGATCAGAGCCGTGACCATCGACCGCGTGATGTCTTACCTG |
| | | | AACGCCAGC |
| 170 | | PCS^{a} | LSGRSDDH |
| 171 | | PCS | ISSGLLSGRSDNH |
| 172 | | PCS | ISSGLLSGRSDQH |
| 173 | | PCS | ISSGLLSGRSDDH |
| 174 | | PCS | LSGRSGNH |
| 175 | | PCS | TSTSGRSANPRG |
| 176 | | PCS | ISSGLLSS |
| 177 | | PCS | QNQALRMA |
| 178 | | PCS | VHMPLGFLGP |
| 179 | | PCS | AVGLLAPP |
| 180 | | PCS | LSGRSDDH |
| 181 | | PCS | LSGRSDIH |
| 182 | | PCS | LSGRSDQH |
| 183 | | PCS | LSGRSDTH |
| 184 | | PCS | LSGRSDYH |
| 185 | | PCS | LSGRSDNP |
| 186 | | PCS | LSGRSANP |
| 187 | | PCS | LSGRSANI |
| 188 | | PCS | LSGRSDNI |
| 189 | | PCS | ISSGLLSGRSDNH |
| 190 | | PCS | ISSGLLSGRSGNH |
| 191 | | PCS | ISSGLLSGRSANPRG |
| 192 | | PCS | AVGLLAPPSGRSANPRG |
| 193 | | PCS | ISSGLLSGRSDDH |
| 194 | | PCS | ISSGLLSGRSDIH |
| 195 | | PCS | ISSGLLSGRSDQH |
| 196 | | PCS | ISSGLLSGRSDTH |
| 197 | | PCS | ISSGLLSGRSDYH |
| 198 | | PCS | ISSGLLSGRSDNP |
| 199 | | PCS | ISSGLLSGRSANP |
| 200 | | PCS | ISSGLLSGRSANI |
| 201 | | PCS | ISSGLLSGRSDNI |
| 202 | | PCS | AVGLLAPPGGLSGRSDNH |
| 203 | | PCS | AVGLLAPPGGLSGRSDDH |
| 204 | | PCS | AVGLLAPPGGLSGRSDIH |
| 205 | | PCS | AVGLLAPPGGLSGRSDQH |
| 206 | | PCS | AVGLLAPPGGLSGRSDTH |
| 207 | | PCS | AVGLLAPPGGLSGRSDYH |
| 208 | | PCS | AVGLLAPPGGLSGRSDNP |
| 209 | | PCS | AVGLLAPPGGLSGRSANP |
| 210 | | PCS | AVGLLAPPGGLSGRSANI |
| 211 | | PCS | AVGLLAPPGGLSGRSDNI |
| 212 | | PCS | PRFKIIGG |
| 213 | | PCS | PRFRIIGG |
| 214 | | PCS | SSRHRRALD |
| 215 | | PCS | RKSSIIIRMRDVVL |
| 216 | | PCS | SSSFDKGKYKKGDDA |
| 217 | | PCS | SSSFDKGKYKRGDDA |
| 218 | | PCS | IEGR |
| 219 | | PCS | IDGR |
| 220 | | PCS | GGSIDGR |
| 221 | | PCS | PLGLWA |
| 222 | | PCS | GPQGIAGQ |
| 223 | | PCS | GPQGLLGA |
| 224 | | PCS | GIAGQ |
| 225 | | PCS | GPLGIAGI |
| 226 | | PCS | GPEGLRVG |
| 227 | | PCS | YGAGLGVV |
| 228 | | PCS | AGLGVVER |
| 229 | | PCS | AGLGISST |
| 230 | | PCS | EPQALAMS |
| 231 | | PCS | QALAMSAI |
| 232 | | PCS | AAYHLVSQ |
| 233 | | PCS | MDAFLESS |
| 234 | | PCS | ESLPVVAV |
| 235 | | PCS | SAPAVESE |
| 236 | | PCS | DVAQFVLT |
| 237 | | PCS | VAQFVLTE |
| 238 | | PCS | AQFVLTEG |
| 239 | | PCS | PVQPIGPQ |
| 240 | | Linker | GSADGG |
| 241 | | Linker | PQGQGGGGSGGGGNSP |
| 242 | | Linker | QGQSGQGG |
| 243 | | WT Hep- | QGKSKREKK |
| | | Loop | |
| 244 | | Hep-Loop^{c} | QGSEK |
| 245 | | Hep-Loop | KDQTE |
| 246 | | Hep-Loop | QDDSE |
| 247 | | Hep-Loop | QDQTD |
| 248 | | Hep-Loop | QGEKK |
| 249 | | Hep-Loop | RDDSE |
| 250 | | Hep-Loop | QGSQEKK |
| 251 | | Hep-Loop | QGESKQEKK |
| 252 | | IL12Rβ1 | MEPLVTWVVPLLFLFLLSRQGAACRTSECCFQDPPYPDADSGSASGPRDLR |
| | | Uniprot | CYRISSDRYECSWQYEGPTAGVSHFLRCCLSSGRCCYFAAGSATRLQFSDQ |
| | | P42701 | AGVSVLYTVTLWVESWARNQTEKSPEVTLQLYNSVKYEPPLGDIKVSKLA |
| | | | GQLRMEWETPDNQVGAEVQFRHRTPSSPWKLGDCGPQDDDTESCLCPLE |
| | | | MNVAQEFQLRRRQLGSQGSSWSKWSSPVCVPPENPPQPQVRFSVEQLGQD |
| | | | GRRRLTLKEQPTQLELPEGCQGLAPGTEVTYRLQLHMLSCPCKAKATRTL |
| | | | HLGKMPYLSGAAYNVAVISSNQFGPGLNQTWHIPADTHTEPVALNISVGTN |
| | | | GTTMYWPARAQSMTYCIEWQPVGQDGGLATCSLTAPQDPDPAGMATYS |
| | | | WSRESGAMGQEKCYYITIFASAHPEKLTLWSTVLSTYHFGGNASAAGTPH |
| | | | HVSVKNHSLDSVSVDWAPSLLSTCPGVLKEYVVRCRDEDSKQVSEHPVQP |
| | | | TETQVTLSGLRAGVAYTVQVRADTAWLRGVWSQPQRFSIEVQVSDWLIFF |
| | | | ASLGSFLSILLVGVLGYLGLNRAARHLCPPLPTPCASSAIEFPGGKETWQWI |
| | | | NPVDFQEEASLQEALVVEMSWDKGERTEPLEKTELPEGAPELALDTELSLE |
| | | | DGDRCKAKM |
| 253 | | IL12Rβ2 | MAHTFRGCSLAFMFIITWLLIKAKIDACKRGDVTVKPSHVILLGSTVNITCS |
| | | Uniprot | LKPRQGCFHYSRRNKLILYKFDRRINFHHGHSLNSQVTGLPLGTTLFVCKL |
| | | Q99665 | ACINSDEIQICGAEIFVGVAPEQPQNLSCIQKGEQGTVACTWERGRDTHLYT |
| | | | EYTLQLSGPKNLTWQKQCKDIYCDYLDFGINLTPESPESNFTAKVTAVNSL |
| | | | GSSSSLPSTFTFLDIVRPLPPWDIRIKFQKASVSRCTLYWRDEGLVLLNRLRY |
| | | | RPSNSRLWNMVNVTKAKGRHDLLDLKPFTEYEFQISSKLHLYKGSWSDWS |
| | | | ESLRAQTPEEEPTGMLDVWYMKRHIDYSRQQISLFWKNLSVSEARGKILHY |
| | | | QVTLQELTGGKAMTQNITGHTSWTTVIPRTGNWAVAVSAANSKGSSLPTRI |
| | | | NIMNLCEAGLLAPRQVSANSEGMDNILVTWQPPRKDPSAVQEYVVEWREL |
| | | | HPGGDTQVPLNWLRSRPYNVSALISENIKSYICYEIRVYALSGDQGGCSSIL |
| | | | GNSKHKAPLSGPHINAITEEKGSILISWNSIPVQEQMGCLLHYRIYWKERDS |
| | | | NSQPQLCEIPYRVSQNSHPINSLQPRVTYVLWMTALTAAGESSHGNEREFC |
| | | | LQGKANWMAFVAPSICIAIIMVGIFSTHYFQQKVFVLLAALRPQWCSREIPD |
| | | | PANSTCAKKYPIAEEKTQLPLDRLLIDWPTPEDPEPLVISEVLHQVTPVFRHP |
| | | | PCSNWPQREKGIQGHQASEKDMMHSASSPPPPRALQAESRQLVDLYKVLE |
| | | | SRGSDPKPENPACPWTVLPAGDLPTHDGYLPSNIDDLPSHEAPLADSLEELE |
| | | | PQHISLSVFPSSSLHPLTFSCGDKLTLDQLKMRCDSLML |
| 254 | | Human | APELLGGPSVFLFPPKPKDTLMISRTPEVTCVVVDVSHEDPEVKFNWYVDG |
| | | IgG1 Fc | VEVHNAKTKPREEQYNSTYRVVSVLTVLHQDWLNGKEYKCKVSNKALPA |
| | | 231-447 | PIEKTISKAKGQPREPQVYTLPPSRDELTKNQVSLTCLVKGFYPSDIAVEWE |
| | | (EU- | SNGQPENNYKTTPPVLDSDGSFFLYSKLTVDKSRWQQGNVFSCSVMHEAL |
| | | numbering) | HNHYTQKSLSLSPGK |
| 255 | | h6F6 VH | QVQLVQSGAEVKKPGASVKVSCKASGYTFTDYYLHWVRQAPGQGLEWM |
| | | | GWIDPENGDTEYAPKFQGRVTMTTDTSTSTAYMELRSLRSDDTAVYYCNA |
| | | | NKELRYFDVWGQGTMVTVSS |
| 256 | | h6F6 VLAR | EIVMTQSPATLSVSPGERATLSCRASQSISINLHWYQQKPGQAPRLLIYFAS |
| | | | QSISGIPARFSGSGSGTEFTLTISSLQSEDFAVYYCQQSNSFPLTFGGGTKVEI |
| | | | K |
| 257 | | h6F6 | DYYLH |
| | | VHCDR1 | |
| 258 | | h6F6 | WIDPENGDTEYAPKFQG |
| | | VHCDR2 | |
| 259 | | h6F6 | NKELRYFDV |
| | | VHCDR3 | |
| 260 | | h6F6 | RASQSISINLH |
| | | VLCDR1 | |
| 261 | | h6F6 | FASQSIS |
| | | VLCDR2 | |
| 262 | | h6F6 | QQSNSFPLT |
| | | VLCDR3 | |
| 263 | | IL23R Full | MNQVTIQWDAVIALYILFSWCHGGITNINCSGHIWVEPATIFKMGMNISIYC |
| | | Uniprot | QAAIKNCQPRKLHFYKNGIKERFQITRINKTTARLWYKNFLEPHASMYCTA |
| | | Q5VWK5 | ECPKHFQETLICGKDISSGYPPDIPDEVTCVIYEYSGNMTCTWNAGKLTYID |
| | | | TKYVVHVKSLETEEEQQYLTSSYINISTDSLQGGKKYLVWVQAANALGME |
| | | | ESKQLQIHLDDIVIPSAAVISRAETINATVPKTIIYWDSQTTIEKVSCEMRYK |
| | | | ATTNQTWNVKEFDTNFTYVQQSEFYLEPNIKYVFQVRCQETGKRYWQPW |
| | | | SSLFFHKTPETVPQVTSKAFQHDTWNSGLTVASISTGHLTSDNRGDIGLLLG |
| | | | MIVFAVMLSILSLIGIFNRSFRTGIKRRILLLIPKWLYEDIPNMKNSNVVKML |
| | | | QENSELMNNNSSEQVLYVDPMITEIKEIFIPEHKPTDYKKENTGPLETRDYP |
| | | | QNSLFDNTTVVYIPDLNTGYKPQISNFLPEGSHLSNNNEITSLTLKPPVDSLD |
| | | | SGNNPRLQKHPNFAFSVSSVNSLSNTIFLGELSLILNQGECSSPDIQNSVEEE |
| | | | TTMLLENDSPSETIPEQTLLPDEFVSCLGIVNEELPSINTYFPQNILESHFNRIS |
| | | | LLEK |
| 264 | | IL23R_ECD_ | GITNINCSGHIWVEPATIFKMGMNISIYCQAAIKNCQPRKLHFYKNGIKERF |
| | | 24-355 | QITRINKTTARLWYKNFLEPHASMYCTAECPKHFQETLICGKDISSGYPPDIP |
| | | Q5VWK5 | DEVTCVIYEYSGNMTCTWNAGKLTYIDTKYVVHVKSLETEEEQQYLTSSYI |
| | | | NISTDSLQGGKKYLVWVQAANALGMEESKQLQIHLDDIVIPSAAVISRAETI |
| | | | NATVPKTIIYWDSQTTIEKVSCEMRYKATTNQTWNVKEFDTNFTYVQQSEF |
| | | | YLEPNIKYVFQVRCQETGKRYWQPWSSLFFHKTPETVPQVTSKAFQHDTW |
| | | | NSGLTVASISTGHLTSDNRGDIG |
| 265 | | IL23R_ECD_ | GITNINCSGHIWVEPATIFKMGMNISIYCQAAIKNCQPRKLHFYKNGIKERF |
| | | 24-318 (Ig, | QITRINKTTARLWYKNFLEPHASMYCTAECPKHFQETLICGKDISSGYPPDIP |
| | | FN3-1 and | DEVTCVIYEYSGNMTCTWNAGKLTYIDTKYVVHVKSLETEEEQQYLTSSYI |
| | | FN3-2 | NISTDSLQGGKKYLVWVQAANALGMEESKQLQIHLDDIVIPSAAVISRAETI |
| | | domains) | NATVPKTIIYWDSQTTIEKVSCEMRYKATTNQTWNVKEFDTNFTYVQQSEF |
| | | Q5VWK5 | YLEPNIKYVFQVRCQETGKRYWQPWSSLFFHKTPET |
| 266 | | IL23R_ECD | GITNINCSGHIWVEPATIFKMGMNISIYCQAAIKNCQPRKLHFYKNGIKERF |
| | | _24-126(Ig | QITRINKTTARLWYKNFLEPHASMYCTAECPKHFQETLICGKDISSGYPPD |
| | | domain) | |
| | | Q5VWK5 | |
| 267 | | p40-L-p35 | IWELKKDVYVVELDWYPDAPGEMVVLTCDTPEEDGITWTLDQSSEVLGSG |
| | | (See | KTLTIQVKEFGDAGQYTCHKGGEVLSHSLLLLHKKEDGIWSTDILKDQKEP |
| | | CL_# 17876) | KNKTFLRCEAKNYSGRFTCWWLTTISTDLTFSVKSSRGSSDPQGVTCGAAT |
| | | | LSAERVRGDNKEYEYSVECQEDSACPAAEESLPIEVMVDAVHKLKYENYT |
| | | | SSFFIRDIIKPDPPKNLQLKPLKNSRQVEVSWEYPDTWSTPHSYFSLTFCVQV |
| | | | QGKSKREKKDRVFTDKTSATVICRKNASISVRAQDRYYSSSWSEWASVPCS |
| | | | GGGGSGGGGSGGGGSRNLPVATPDPGMFPCLHHSQNLLRAVSNMLQKAR |
| | | | QTLEFYPCTSEEIDHEDITKDKTSTVEACLPLELTKNESCLNSRETSFITNGSC |
| | | | LASRKTSFMMALCLSSIYEDLKMYQVEFKTMNAKLLMDPKRQIFLDQNML |
| | | | AVIDELMQALNFNSETVPQKSSLEEPDFYKTKIKLCILLHAFRIRAVTIDRV |
| | | | MSYLNAS |
| 268 | | p4O-L- | IWELKKDVYVVELDWYPDAPGEMVVLTCDTPEEDGITWTLDQSSEVLGSG |
| | | p35AR (See | KTLTIQVKEFGDAGQYTCHKGGEVLSHSLLLLHKKEDGIWSTDILKDQKEP |
| | | CL_#22279) | KNKTFLRCEAKNYSGRFTCWWLTTISTDLTFSVKSSRGSSDPQGVTCGAAT |
| | | | LSAERVRGDNKEYEYSVECQEDSACPAAEESLPIEVMVDAVHKLKYENYT |
| | | | SSFFIRDIIKPDPPKNLQLKPLKNSRQVEVSWEYPDTWSTPHSYFSLTFCVQV |
| | | | QGKSKREKKDRVFTDKTSATVICRKNASISVRAQDRYYSSSWSEWASVPCS |
| | | | GGGGSGGGGSGGGGSNLPVATPDPGMFPCLHHSQNLLRAVSNMLQKARQ |
| | | | TLEFYPCTSEEIDHEDITKDKTSTVEACLPLELTKNESCLNSRETSFITNGSCL |
| | | | ASRKTSFMMALCLSSIYEDLKMYQVEFKTMNAKLLMDPKRQIFLDQNML |
| | | | AVIDELMQALNFNSETVPQKSSLEEPDFYKTKIKLCILLHAFRIRAVTIDRV |
| | | | MSYLNAS |
| 269 | | p40HEP-L- | IWELKKDVYVVELDWYPDAPGEMVVLTCDTPEEDGITWTLDQSSEVLGSG |
| | | p35ΔR (Hep | KTLTIQVKEFGDAGQYTCHKGGEVLSHSLLLLHKKEDGIWSTDILKDQKEP |
| | | graft as in | KNKTFLRCEAKNYSGRFTCWWLTTISTDLTFSVKSSRGSSDPQGVTCGAAT |
| | | v30818- | LSAERVRGDNKEYEYSVECQEDSACPAAEESLPIEVMVDAVHKLKYENYT |
| | | See | SSFFIRDIIKPDPPKNLQLKPLKNSRQVEVSWEYPDTWSTPHSYFSLTFCVQV |
| | | CL_#22291) | QGESKQEKKDRVFTDKTSATVICRKNASISVRAQDRYYSSSWSEWASVPCS |
| | | | GGGGSGGGGSGGGGSNLPVATPDPGMFPCLHHSQNLLRAVSNMLQKARQ |
| | | | TLEFYPCTSEEIDHEDITKDKTSTVEACLPLELTKNESCLNSRETSFITNGSCL |
| | | | ASRKTSFMMALCLSSIYEDLKMYQVEFKTMNAKLLMDPKRQIFLDQNML |
| | | | AVIDELMQALNFNSETVPQKSSLEEPDFYKTKIKLCILLHAFRIRAVTIDRV |
| | | | MSYLNAS |
| 270 | | h6F6 VL | EIVMTQSPATLSVSPGERATLSCRASQSISINLHWYQQKPGQAPRLLIYFAS |
| | | Full | QSISGIPARFSGSGSGTEFTLTISSLQSEDFAVYYCQQSNSFPLTFGGGTKVEI |
| | | | KR |
| 271 | | | anti-CD3 paratope was described in |
| | | | US20150232557A1 (VL SEQ ID NO: 271). |
| 272 | | | anti-CD3 paratope was described in |
| | | | US20150232557A1 (VH SEQ ID NO: 272). |
| 273 | | | DIQMTQSPSSLSASVGDRVTITCRASQDVNTAVAWYQQK |
| | | | PGKAPKLLIYSASFLYSGVPSRFSGSRSGTDFTLTISSL |
| | | | QPEDFATYYCQQHYTTPPTFGQGTKVEIKGGSGGGSGGG |
| | | | SGGGSGGGSGEVQLVESGGGLVQPGGSLRLSCAASGFNI |
| | | | KDTYIHWVRQAPGKGLEWVARIYPTNGYTRYADSVKGRF |
| | | | TISADTSKNTAYLQMNSLRAEDTAVYYCSRWGGDGFYAM |
| | | | DYWGQGTLVTVSS |
| 274 | | | GQPREPQVYVYPPSRDELTKNQVSLTCLVKGFYPSDIAV |
| | | | EWESNGQPENNYKTTPPVLDSDGSFALVSKLTVDKSRWQ |
| | | | QGNVFSCSVMHEALHNHYTQKSLSLSPG |
| 275 | | | GQPREPQVYVLPPSRDELTKNQVSLLCLVKGFYPSDIAV |
| | | | EWESNGQPENNYLTWPPVLDSDGSFFLYSKLTVDKSRWQ |
| | | | QGNVFSCSVMHEALHNHYTQKSLSLSPG |
| 276 | | | APEAAGGPSVFLFPPKPKDTLMISRTPEVTCVVVSVSHE |
| | | | DPEVKFNWYVDGVEVHNAKTKPREEQYNSTYRVVSVLTV |
| | | | LHQDWLNGKEYKCKVSNKALPAPIEKTISKAK |
| 277 | | | NPPTFSPALLVVTEGDNATFTCSFSNTSESFVLNWYRMS |
| | | | PSNQTDKLAAFPEDRSQPGQDCRFRVTQLPNGRDFHMSV |
| | | | VRARRNDSGTYLCGAISLAPKAQIKESLRAELRVTE |
| 278 | | | AFTVTVPKDLYVVEYGSNMTIECKFPVEKQLDLAALIVY |
| | | | WEMEDKNIIQFVHGEEDLKVQHSSYRQRARLLKDQLSLG |
| | | | NAALQITDVKLQDAGVYRCMISYGGADYKRITVKVNA |
| 279 | | | NPPTFSPALLVVTEGDNATFTCSFSNTSESFHVVWHRES |
| | | | PSGQTDTLAAFPEDRSQPGQDARFRVTQLPNGRDFHMSV |
| | | | VRARRNDSGTYVCGVISLAPKIQIKESLRAELRVTE |
| 280 | 12985 | full AA | DIQMTQSPSSLSASVGDRVTMTCSASSSVSYMNWYQQKPGKAPKRWIYDS |
| | | | SKLASGVPARFSGSGSGTDYTLTISSLQPEDFATYYCQQWSRNPPTFGGGT |
| | | | KLQITRTVAAPSVFIFPPSDEQLKSGTASVVCLLNNFYPREAKVQWKVDNA |
| | | | LQSGNSQESVTEQDSKDSTYSLSSTLTLSKADYEKHKVYACEVTHQGLSSP |
| | | | VTKSFNRGEC |
| 281 | 12989 | full AA | QVQLVESGGGVVQPGRSLRLSCKASGYTFTRSTMHWVRQAPGQGLEWIG |
| | | | YINPSSAYTNYNQKFKDRFTISADKSKSTAFLQMDSLRPEDTGVYFCARPQ |
| | | | VHYDYNGFPYWGQGTPVTVSSASTKGPSVFPLAPSSKSTSGGTAALGCLV |
| | | | KDYFPEPVTVSWNSGALTSGVHTFPAVLQSSGLYSLSSVVTVPSSSLGTQT |
| | | | YICNVNHKPSNTKVDKKVEPKSCDKTHTCPPCPAPEAAGGPSVFLFPPKPK |
| | | | DTLMISRTPEVTCVVVSVSHEDPEVKFNWYVDGVEVHNAKTKPREEQYNS |
| | | | TYRVVSVLTVLHQDWLNGKEYKCKVSNKALPAPIEKTISKAKGQPREPQV |
| | | | YVYPPSRDELTKNQVSLTCLVKGFYPSDIAVEWESNGQPENNYKTTPPVLD |
| | | | SDGSFALVSKLTVDKSRWQQGNVFSCSVMHEALHNHYTQKSLSLSPG |
| 282 | 21490 | full AA | DIQMTQSPSSLSASVGDRVTITCRASQDVNTAVAWYQQKPGKAPKLLIYSA |
| | | | SFLYSGVPSRFSGSRSGTDFTLTISSLQPEDFATYYCQQHYTTPPTFGQGTKV |
| | | | EIKGGSGGGSGGGSGGGSGGGSGEVQLVESGGGLVQPGGSLRLSCAASGF |
| | | | NIKDTYIHWVRQAPGKGLEWVARIYPTNGYTRYADSVKGRFTISADTSKN |
| | | | TAYLQMNSLRAEDTAVYYCSRWGGDGFYAMDYWGQGTLVTVSSEPKSS |
| | | | DKTHTCPPCPAPEAAGGPSVFLFPPKPKDTLMISRTPEVTCVVVSVSHEDPE |
| | | | VKFNWYVDGVEVHNAKTKPREEQYNSTYRVVSVLTVLHQDWLNGKEYK |
| | | | CKVSNKALPAPIEKTISKAKGQPREPQVYVLPPSRDELTKNQVSLLCLVKGF |
| | | | YPSDIAVEWESNGQPENNYLTWPPVLDSDGSFFLYSKLTVDKSRWQQGNV |
| | | | FSCSVMHEALHNHYTQKSLSLSPG |
| 283 | 22080 | full AA | NPPTFSPALLVVTEGDNATFTCSFSNTSESFHVVWHRESPSGQTDTLAAFPE |
| | | | DRSQPGQDARFRVTQLPNGRDFHMSVVRARRNDSGTYVCGVISLAPKIQIK |
| | | | ESLRAELRVTEEAAAKEAAAKQVQLVESGGGVVQPGRSLRLSCKASGYTF |
| | | | TRSTMHWVRQAPGQGLEWIGYINPSSAYTNYNQKFKDRFTISADKSKSTAF |
| | | | LQMDSLRPEDTGVYFCARPQVHYDYNGFPYWGQGTPVTVSSASTKGPSVF |
| | | | PLAPSSKSTSGGTAALGCLVKDYFPEPVTVSWNSGALTSGVHTFPAVLQSS |
| | | | GLYSLSSVVTVPSSSLGTQTYICNVNHKPSNTKVDKKVEPKSCDKTHTCPP |
| | | | CPAPEAAGGPSVFLFPPKPKDTLMISRTPEVTCVVVSVSHEDPEVKFNWYV |
| | | | DGVEVHNAKTKPREEQYNSTYRVVSVLTVLHQDWLNGKEYKCKVSNKAL |
| | | | PAPIEKTISKAKGQPREPQVYVYPPSRDELTKNQVSLTCLVKGFYPSDIAVE |
| | | | WESNGQPENNYKTTPPVLDSDGSFALVSKLTVDKSRWQQGNVFSCSVMH |
| | | | EALHNHYTQKSLSLSPG |
| 284 | 22082 | full AA | NPPTFSPALLVVTEGDNATFTCSFSNTSESFVLNWYRMSPSNQTDKLAAFPE |
| | | | DRSQPGQDSRFRVTQLPNGRDFHMSVVRARRNDSGTYLCGAISLAPKAQIK |
| | | | ESLRAELRVTEEAAAKEAAAKQVQLVESGGGVVQPGRSLRLSCKASGYTF |
| | | | TRSTMHWVRQAPGQGLEWIGYINPSSAYTNYNQKFKDRFTISADKSKSTAF |
| | | | LQMDSLRPEDTGVYFCARPQVHYDYNGFPYWGQGTPVTVSSASTKGPSVF |
| | | | PLAPSSKSTSGGTAALGCLVKDYFPEPVTVSWNSGALTSGVHTFPAVLQSS |
| | | | GLYSLSSVVTVPSSSLGTQTYICNVNHKPSNTKVDKKVEPKSCDKTHTCPP |
| | | | CPAPEAAGGPSVFLFPPKPKDTLMISRTPEVTCVVVSVSHEDPEVKFNWYV |
| | | | DGVEVHNAKTKPREEQYNSTYRVVSVLTVLHQDWLNGKEYKCKVSNKAL |
| | | | PAPIEKTISKAKGQPREPQVYVYPPSRDELTKNQVSLTCLVKGFYPSDIAVE |
| | | | WESNGQPENNYKTTPPVLDSDGSFALVSKLTVDKSRWQQGNVFSCSVMH |
| | | | EALHNHYTQKSLSLSPG |
| 285 | 22083 | full AA | NPPTFSPALLVVTEGDNATFTCSFSNTSESFVLNWYRMSPSNQTDKLAAFPE |
| | | | DRSQPGQDSRFRVTQLPNGRDFHMSVVRARRNDSGTYLCGAISLAPKAQIK |
| | | | ESLRAELRVTEMSGRSANAEAAAKQVQLVESGGGVVQPGRSLRLSCKASG |
| | | | YTFTRSTMHWVRQAPGQGLEWIGYINPSSAYTNYNQKFKDRFTISADKSKS |
| | | | TAFLQMDSLRPEDTGVYFCARPQVHYDYNGFPYWGQGTPVTVSSASTKGP |
| | | | SVFPLAPSSKSTSGGTAALGCLVKDYFPEPVTVSWNSGALTSGVHTFPAVL |
| | | | QSSGLYSLSSVVTVPSSSLGTQTYICNVNHKPSNTKVDKKVEPKSCDKTHT |
| | | | CPPCPAPEAAGGPSVFLFPPKPKDTLMISRTPEVTCVVVSVSHEDPEVKFNW |
| | | | YVDGVEVHNAKTKPREEQYNSTYRVVSVLTVLHQDWLNGKEYKCKVSN |
| | | | KALPAPIEKTISKAKGQPREPQVYVYPPSRDELTKNQVSLTCLVKGFYPSDI |
| | | | AVEWESNGQPENNYKTTPPVLDSDGSFALVSKLTVDKSRWQQGNVFSCSV |
| | | | MHEALHNHYTQKSLSLSPG |
| 286 | 22086 | full AA | NPPTFSPALLVVTEGDNATFTCSFSNTSESFVLNWYRMSPSNQTDKLAAFPE |
| | | | DRSQPGQDSRFRVTQLPNGRDFHMSVVRARRNDSGTYLCGAISLAPKAQIK |
| | | | ESLRAELRVTEEAAAKEAAAKMSGRSANAQVQLVESGGGVVQPGRSLRLS |
| | | | CKASGYTFTRSTMHWVRQAPGQGLEWIGYINPSSAYTNYNQKFKDRFTIS |
| | | | ADKSKSTAFLQMDSLRPEDTGVYFCARPQVHYDYNGFPYWGQGTPVTVSS |
| | | | ASTKGPSVFPLAPSSKSTSGGTAALGCLVKDYFPEPVTVSWNSGALTSGVH |
| | | | TFPAVLQSSGLYSLSSVVTVPSSSLGTQTYICNVNHKPSNTKVDKKVEPKSC |
| | | | DKTHTCPPCPAPEAAGGPSVFLFPPKPKDTLMISRTPEVTCVVVSVSHEDPE |
| | | | VKFNWYVDGVEVHNAKTKPREEQYNSTYRVVSVLTVLHQDWLNGKEYK |
| | | | CKVSNKALPAPIEKTISKAKGQPREPQVYVYPPSRDELTKNQVSLTCLVKG |
| | | | FYPSDIAVEWESNGQPENNYKTTPPVLDSDGSFALVSKLTVDKSRWQQGN |
| | | | VFSCSVMHEALHNHYTQKSLSLSPG |
| 287 | 22091 | full AA | AFTVTVPKDLYVVEYGSNMTIECKFPVEKQLDLAALIVYWEMEDKNIIQFV |
| | | | HGEEDLKVQHSSYRQRARLLKDQLSLGNAALQITDVKLQDAGVYRCMISY |
| | | | GGADYKRITVKVNAEAAAKEAAAKDIQMTQSPSSLSASVGDRVTMTCSAS |
| | | | SSVSYMNWYQQKPGKAPKRWIYDSSKLASGVPARFSGSGSGTDYTLTISSL |
| | | | QPEDFATYYCQQWSRNPPTFGGGTKLQITRTVAAPSVFIFPPSDEQLKSGTA |
| | | | SVVCLLNNFYPREAKVQWKVDNALQSGNSQESVTEQDSKDSTYSLSSTLT |
| | | | LSKADYEKHKVYACEVTHQGLSSPVTKSFNRGEC |
| 288 | 22092 | full AA | AFTVTVPKDLYVVEYGSNMTIECKFPVEKQLDLAALQVFWMMEDKNIIQF |
| | | | VHGEEDLKVQHSSYRQRARLLKDQLSLGNAALQITDVKLQDAGVYTCLIA |
| | | | YKGADYKRITVKVNAEAAAKEAAAKDIQMTQSPSSLSASVGDRVTMTCSA |
| | | | SSSVSYMNWYQQKPGKAPKRWIYDSSKLASGVPARFSGSGSGTDYTLTISS |
| | | | LQPEDFATYYCQQWSRNPPTFGGGTKLQITRTVAAPSVFIFPPSDEQLKSGT |
| | | | ASVVCLLNNFYPREAKVQWKVDNALQSGNSQESVTEQDSKDSTYSLSSTL |
| | | | TLSKADYEKHKVYACEVTHQGLSSPVTKSFNRGEC |
| 289 | 22094 | full AA | AFTVTVPKDLYVVEYGSNMTIECKFPVEKQLDLAALIVYWEMEDKNIIQFV |
| | | | HGEEDLKVQHSSYRQRARLLKDQLSLGNAALQITDVKLQDAGVYRCMISY |
| | | | GGADYKRITVKVNAEAAAKMSGRSANADIQMTQSPSSLSASVGDRVTMT |
| | | | CSASSSVSYMNWYQQKPGKAPKRWIYDSSKLASGVPARFSGSGSGTDYTL |
| | | | TISSLQPEDFATYYCQQWSRNPPTFGGGTKLQITRTVAAPSVFIFPPSDEQLK |
| | | | SGTASVVCLLNNFYPREAKVQWKVDNALQSGNSQESVTEQDSKDSTYSLS |
| | | | STLTLSKADYEKHKVYACEVTHQGLSSPVTKSFNRGEC |
| 290 | 22096 | full AA | AFTVTVPKDLYVVEYGSNMTIECKFPVEKQLDLAALIVYWEMEDKNIIQFV |
| | | | HGEEDLKVQHSSYRQRARLLKDQLSLGNAALQITDVKLQDAGVYRCMISY |
| | | | GGADYKRITVKVNAEAAAKEAAAKMSGRSANADIQMTQSPSSLSASVGD |
| | | | RVTMTCSASSSVSYMNWYQQKPGKAPKRWIYDSSKLASGVPARFSGSGSG |
| | | | TDYTLTISSLQPEDFATYYCQQWSRNPPTFGGGTKLQITRTVAAPSVFIFPPS |
| | | | DEQLKSGTASVVCLLNNFYPREAKVQWKVDNALQSGNSQESVTEQDSKD |
| | | | STYSLSSTLTLSKADYEKHKVYACEVTHQGLSSPVTKSFNRGEC |
| 291 | 12985 | full nt | GACATCCAGATGACACAGAGCCCAAGCTCCCTGAGCGCCTCCGTGGGC |
| | | | GATAGGGTGACCATGACATGCTCTGCCTCTAGCTCCGTGAGCTACATGA |
| | | | ACTGGTATCAGCAGAAGCCAGGCAAGGCCCCCAAGCGGTGGATCTACG |
| | | | ACTCTAGCAAGCTGGCCTCCGGAGTGCCCGCCAGATTTTCTGGCAGCGG |
| | | | CTCCGGCACCGACTATACCCTGACAATCTCCTCTCTGCAGCCTGAGGAT |
| | | | TTCGCCACATACTATTGTCAGCAGTGGTCTAGGAATCCCCCTACCTTTG |
| | | | GCGGCGGCACAAAGCTGCAGATCACCCGCACAGTGGCGGCGCCCAGTG |
| | | | TCTTCATTTTTCCCCCTAGCGACGAACAGCTGAAGTCTGGGACAGCCAG |
| | | | TGTGGTCTGTCTGCTGAACAACTTCTACCCTAGAGAGGCTAAAGTGCAG |
| | | | TGGAAGGTCGATAACGCACTGCAGTCCGGAAATTCTCAGGAGAGTGTG |
| | | | ACTGAACAGGACTCAAAAGATAGCACCTATTCCCTGTCAAGCACACTG |
| | | | ACTCTGAGCAAGGCCGACTACGAGAAGCATAAAGTGTATGCTTGTGAA |
| | | | GTCACCCACCAGGGGCTGAGTTCACCAGTCACAAAATCATTCAACAGA |
| | | | GGGGAGTGC |
| 292 | 12989 | full nt | CAGGTGCAGCTGGTGGAGTCTGGCGGCGGCGTGGTGCAGCCCGGCAGA |
| | | | AGCCTGCGGCTGAGCTGCAAGGCCTCTGGCTACACCTTTACAAGGAGCA |
| | | | CCATGCACTGGGTGCGCCAGGCCCCTGGACAGGGCCTGGAGTGGATCG |
| | | | GCTATATCAACCCAAGCTCCGCCTACACAAACTATAATCAGAAGTTCAA |
| | | | GGACCGGTTTACCATCAGCGCCGATAAGTCCAAGTCTACAGCCTTCCTG |
| | | | CAGATGGACTCCCTGCGGCCAGAGGATACAGGCGTGTACTTCTGTGCCA |
| | | | GACCCCAGGTGCACTACGACTATAATGGCTTTCCCTATTGGGGCCAGGG |
| | | | CACCCCTGTGACAGTGTCTAGCGCTAGCACTAAGGGGCCTTCCGTGTTT |
| | | | CCACTGGCTCCCTCTAGTAAATCCACCTCTGGAGGCACAGCTGCACTGG |
| | | | GATGTCTGGTGAAGGATTACTTCCCTGAACCAGTCACAGTGAGTTGGAA |
| | | | CTCAGGGGCTCTGACAAGTGGAGTCCATACTTTTCCCGCAGTGCTGCAG |
| | | | TCAAGCGGACTGTACTCCCTGTCCTCTGTGGTCACCGTGCCTAGTTCAA |
| | | | GCCTGGGCACCCAGACATATATCTGCAACGTGAATCACAAGCCATCAA |
| | | | ATACAAAAGTCGACAAGAAAGTGGAGCCCAAGAGCTGTGATAAAACTC |
| | | | ATACCTGCCCACCTTGTCCGGCGCCAGAGGCTGCAGGAGGACCAAGCG |
| | | | TGTTCCTGTTTCCACCCAAGCCTAAAGACACACTGATGATTTCCCGAAC |
| | | | CCCCGAAGTCACATGCGTGGTCGTGTCTGTGAGTCACGAGGACCCTGAA |
| | | | GTCAAGTTCAACTGGTACGTGGATGGCGTCGAGGTGCATAATGCCAAG |
| | | | ACTAAACCTAGGGAGGAACAGTACAACTCAACCTATCGCGTCGTGAGC |
| | | | GTCCTGACAGTGCTGCACCAGGATTGGCTGAACGGCAAAGAATATAAG |
| | | | TGCAAAGTGAGCAATAAGGCCCTGCCCGCTCCTATCGAGAAAACCATTT |
| | | | CCAAGGCTAAAGGGCAGCCTCGCGAACCACAGGTCTACGTGTATCCTCC |
| | | | AAGCCGGGACGAGCTGACAAAGAACCAGGTCTCCCTGACTTGTCTGGT |
| | | | GAAAGGGTTTTACCCTAGTGATATCGCTGTGGAGTGGGAATCAAATGG |
| | | | ACAGCCAGAGAACAATTATAAGACTACCCCCCCTGTGCTGGACAGTGA |
| | | | TGGGTCATTCGCACTGGTCTCCAAGCTGACAGTGGACAAATCTCGGTGG |
| | | | CAGCAGGGAAATGTCTTTTCATGTAGCGTGATGCATGAAGCACTGCACA |
| | | | ACCATTACACCCAGAAGTCACTGTCACTGTCACCAGGA |
| 293 | 21490 | full nt | GACATCCAGATGACACAGTCTCCTAGCTCCCTGTCTGCCAGCGTGGGCG |
| | | | ACAGGGTGACCATCACATGCAGGGCCAGCCAGGATGTGAACACCGCCG |
| | | | TGGCCTGGTACCAGCAGAAGCCTGGCAAGGCCCCAAAGCTGCTGATCT |
| | | | ACTCCGCCTCTTTCCTGTATAGCGGCGTGCCTTCCCGGTTTAGCGGCTCC |
| | | | AGATCTGGCACCGACTTCACCCTGACAATCTCTAGCCTGCAGCCAGAGG |
| | | | ATTTTGCCACATACTATTGCCAGCAGCACTATACCACACCCCCTACCTTC |
| | | | GGCCAGGGCACAAAGGTGGAGATCAAGGGAGGCTCTGGAGGAGGCAG |
| | | | CGGAGGAGGCTCCGGAGGAGGCTCTGGCGGCGGCAGCGGCGAGGTGCA |
| | | | GCTGGTGGAGTCCGGCGGCGGCCTGGTGCAGCCCGGCGGCTCCCTGCG |
| | | | GCTGTCTTGTGCCGCCAGCGGCTTCAACATCAAGGATACCTACATCCAC |
| | | | TGGGTGCGGCAGGCCCCAGGCAAGGGACTGGAGTGGGTGGCCAGAATC |
| | | | TATCCCACCAATGGCTACACACGGTATGCCGACAGCGTGAAGGGCCGG |
| | | | TTCACCATCTCCGCCGATACCTCTAAGAACACAGCCTACCTGCAGATGA |
| | | | ATAGCCTGAGGGCCGAGGACACAGCCGTGTACTATTGTTCCCGCTGGGG |
| | | | AGGCGACGGCTTCTACGCAATGGATTATTGGGGCCAGGGCACCCTGGT |
| | | | GACAGTGTCCTCTGAGCCTAAGAGCTCCGATAAGACCCACACATGCCCA |
| | | | CCCTGTCCGGCGCCAGAGGCCGCCGGAGGACCAAGCGTGTTCCTGTTTC |
| | | | CACCCAAGCCCAAGGACACCCTGATGATCTCCCGGACCCCAGAGGTGA |
| | | | CATGCGTGGTGGTGAGCGTGTCCCACGAGGACCCCGAGGTGAAGTTTA |
| | | | ACTGGTACGTGGATGGCGTGGAGGTGCACAATGCCAAGACAAAGCCCC |
| | | | GGGAGGAGCAGTACAATTCTACCTATAGAGTGGTGAGCGTGCTGACAG |
| | | | TGCTGCACCAGGATTGGCTGAACGGCAAGGAGTATAAGTGTAAGGTGA |
| | | | GCAATAAGGCCCTGCCAGCCCCCATCGAGAAGACCATCTCCAAGGCCA |
| | | | AGGGCCAGCCTCGCGAACCACAGGTCTACGTGCTGCCCCCTAGCCGCG |
| | | | ACGAACTGACTAAAAATCAGGTCTCTCTGCTGTGTCTGGTCAAAGGATT |
| | | | CTACCCTTCCGACATCGCCGTGGAGTGGGAAAGTAACGGCCAGCCCGA |
| | | | GAACAATTACCTGACCTGGCCCCCTGTGCTGGACTCTGATGGGAGTTTC |
| | | | TTTCTGTATTCAAAGCTGACAGTCGATAAAAGCCGGTGGCAGCAGGGC |
| | | | AATGTGTTCAGCTGCTCCGTCATGCACGAAGCACTGCACAACCATTACA |
| | | | CTCAGAAGTCCCTGTCCCTGTCACCTGGC |
| 294 | 22080 | full nt | AACCCCCCTACCTTTTCCCCAGCCCTGCTGGTGGTGACAGAGGGCGACA |
| | | | ACGCCACCTTCACATGCTCTTTTAGCAATACATCCGAGTCTTTCCACGTG |
| | | | GTGTGGCACCGGGAGAGCCCATCCGGACAGACCGATACACTGGCCGCC |
| | | | TTTCCAGAGGACAGATCTCAGCCAGGACAGGATGCAAGGTTCCGCGTG |
| | | | ACCCAGCTGCCAAACGGCAGGGACTTTCACATGTCTGTGGTGCGCGCCC |
| | | | GGAGAAATGATAGCGGCACATACGTGTGCGGCGTGATCTCCCTGGCCC |
| | | | CTAAGATCCAGATCAAGGAGAGCCTGAGGGCAGAGCTGAGGGTGACCG |
| | | | AGGAGGCTGCCGCCAAGGAGGCTGCCGCCAAGCAGGTGCAGCTGGTGG |
| | | | AGTCTGGAGGAGGAGTGGTGCAGCCCGGCAGAAGCCTGCGGCTGAGCT |
| | | | GTAAGGCCTCCGGCTACACCTTCACACGGAGCACCATGCACTGGGTGA |
| | | | GACAGGCCCCCGGACAGGGACTGGAGTGGATCGGCTATATCAATCCTA |
| | | | GCTCCGCCTACACAAACTATAATCAGAAGTTTAAGGACCGGTTTACCAT |
| | | | CAGCGCCGATAAGTCCAAGTCTACAGCCTTCCTGCAGATGGACTCCCTG |
| | | | CGGCCAGAGGATACAGGCGTGTACTTCTGTGCCAGACCCCAGGTGCACT |
| | | | ACGACTATAACGGCTTTCCCTATTGGGGCCAGGGCACCCCTGTGACAGT |
| | | | GTCTAGCGCTAGCACTAAGGGGCCTTCCGTGTTTCCACTGGCTCCCTCT |
| | | | AGTAAATCCACCTCTGGAGGCACAGCTGCACTGGGATGTCTGGTGAAG |
| | | | GATTACTTCCCTGAACCAGTCACAGTGAGTTGGAACTCAGGGGCTCTGA |
| | | | CAAGTGGAGTCCATACTTTTCCCGCAGTGCTGCAGTCAAGCGGACTGTA |
| | | | CTCCCTGTCCTCTGTGGTCACCGTGCCTAGTTCAAGCCTGGGCACCCAG |
| | | | ACATATATCTGCAACGTGAATCACAAGCCATCAAATACAAAAGTCGAC |
| | | | AAGAAAGTGGAGCCCAAGAGCTGTGATAAAACTCATACCTGCCCACCT |
| | | | TGTCCGGCGCCAGAGGCTGCAGGAGGACCAAGCGTGTTCCTGTTTCCAC |
| | | | CCAAGCCTAAAGACACACTGATGATTTCCCGAACCCCCGAAGTCACATG |
| | | | CGTGGTCGTGTCTGTGAGTCACGAGGACCCTGAAGTCAAGTTCAACTGG |
| | | | TACGTGGATGGCGTCGAGGTGCATAATGCCAAGACTAAACCTAGGGAG |
| | | | GAACAGTACAACTCAACCTATCGCGTCGTGAGCGTCCTGACAGTGCTGC |
| | | | ACCAGGATTGGCTGAACGGCAAAGAATATAAGTGCAAAGTGAGCAATA |
| | | | AGGCCCTGCCCGCTCCTATCGAGAAAACCATTTCCAAGGCTAAAGGGC |
| | | | AGCCTCGCGAACCACAGGTCTACGTGTATCCTCCAAGCCGGGACGAGCT |
| | | | GACAAAGAACCAGGTCTCCCTGACTTGTCTGGTGAAAGGGTTTTACCCT |
| | | | AGTGATATCGCTGTGGAGTGGGAATCAAATGGACAGCCAGAGAACAAT |
| | | | TATAAGACTACCCCCCCTGTGCTGGACAGTGATGGGTCATTCGCACTGG |
| | | | TCTCCAAGCTGACAGTGGACAAATCTCGGTGGCAGCAGGGAAATGTCTT |
| | | | TTCATGTAGCGTGATGCATGAAGCACTGCACAACCATTACACCCAGAAG |
| | | | TCACTGTCACTGTCACCAGGA |
| 295 | 22082 | full nt | AATCCCCCTACCTTTAGCCCAGCCCTGCTGGTGGTGACAGAGGGCGACA |
| | | | ACGCCACCTTCACATGCTCTTTTAGCAACACCTCCGAGTCTTTCGTGCTG |
| | | | AATTGGTACAGAATGAGCCCATCCAACCAGACAGATAAGCTGGCCGCC |
| | | | TTTCCAGAGGACAGATCTCAGCCCGGCCAGGATAGCAGGTTCCGCGTG |
| | | | ACCCAGCTGCCCAATGGCAGGGACTTTCACATGTCCGTGGTGCGCGCCC |
| | | | GGAGAAACGATTCTGGCACATATCTGTGCGGAGCCATCAGCCTGGCCCC |
| | | | TAAGGCACAGATCAAGGAGTCCCTGAGGGCAGAGCTGAGGGTGACAGA |
| | | | GGAGGCTGCCGCCAAGGAGGCTGCCGCCAAGCAGGTGCAGCTGGTGGA |
| | | | GTCCGGAGGAGGAGTGGTGCAGCCCGGCAGAAGCCTGCGGCTGAGCTG |
| | | | TAAGGCCTCCGGCTACACCTTCACACGGTCTACCATGCACTGGGTGAGA |
| | | | CAGGCCCCCGGACAGGGACTGGAGTGGATCGGCTATATCAATCCTAGC |
| | | | TCCGCCTACACAAACTATAATCAGAAGTTTAAGGACCGGTTTACCATCA |
| | | | GCGCCGATAAGTCCAAGTCTACAGCCTTCCTGCAGATGGACTCCCTGCG |
| | | | GCCAGAGGATACAGGCGTGTACTTCTGTGCCAGACCCCAGGTGCACTAC |
| | | | GACTATAACGGCTTTCCCTATTGGGGCCAGGGCACCCCTGTGACAGTGT |
| | | | CTAGCGCTAGCACTAAGGGGCCTTCCGTGTTTCCACTGGCTCCCTCTAG |
| | | | TAAATCCACCTCTGGAGGCACAGCTGCACTGGGATGTCTGGTGAAGGAT |
| | | | TACTTCCCTGAACCAGTCACAGTGAGTTGGAACTCAGGGGCTCTGACAA |
| | | | GTGGAGTCCATACTTTTCCCGCAGTGCTGCAGTCAAGCGGACTGTACTC |
| | | | CCTGTCCTCTGTGGTCACCGTGCCTAGTTCAAGCCTGGGCACCCAGACA |
| | | | TATATCTGCAACGTGAATCACAAGCCATCAAATACAAAAGTCGACAAG |
| | | | AAAGTGGAGCCCAAGAGCTGTGATAAAACTCATACCTGCCCACCTTGTC |
| | | | CGGCGCCAGAGGCTGCAGGAGGACCAAGCGTGTTCCTGTTTCCACCCA |
| | | | AGCCTAAAGACACACTGATGATTTCCCGAACCCCCGAAGTCACATGCGT |
| | | | GGTCGTGTCTGTGAGTCACGAGGACCCTGAAGTCAAGTTCAACTGGTAC |
| | | | GTGGATGGCGTCGAGGTGCATAATGCCAAGACTAAACCTAGGGAGGAA |
| | | | CAGTACAACTCAACCTATCGCGTCGTGAGCGTCCTGACAGTGCTGCACC |
| | | | AGGATTGGCTGAACGGCAAAGAATATAAGTGCAAAGTGAGCAATAAGG |
| | | | CCCTGCCCGCTCCTATCGAGAAAACCATTTCCAAGGCTAAAGGGCAGCC |
| | | | TCGCGAACCACAGGTCTACGTGTATCCTCCAAGCCGGGACGAGCTGAC |
| | | | AAAGAACCAGGTCTCCCTGACTTGTCTGGTGAAAGGGTTTTACCCTAGT |
| | | | GATATCGCTGTGGAGTGGGAATCAAATGGACAGCCAGAGAACAATTAT |
| | | | AAGACTACCCCCCCTGTGCTGGACAGTGATGGGTCATTCGCACTGGTCT |
| | | | CCAAGCTGACAGTGGACAAATCTCGGTGGCAGCAGGGAAATGTCTTTTC |
| | | | ATGTAGCGTGATGCATGAAGCACTGCACAACCATTACACCCAGAAGTC |
| | | | ACTGTCACTGTCACCAGGA |
| 296 | 22083 | full nt | AATCCCCCTACCTTTTCTCCAGCCCTGCTGGTGGTGACAGAGGGCGACA |
| | | | ACGCCACCTTCACATGCTCTTTTAGCAACACCTCCGAGTCTTTCGTGCTG |
| | | | AATTGGTACAGAATGAGCCCATCCAACCAGACAGATAAGCTGGCCGCC |
| | | | TTTCCAGAGGACAGATCCCAGCCCGGCCAGGATTCTAGGTTCCGCGTGA |
| | | | CCCAGCTGCCCAATGGCAGGGACTTTCACATGAGCGTGGTGCGCGCCCG |
| | | | GAGAAACGATTCCGGCACATATCTGTGCGGAGCCATCTCTCTGGCCCCT |
| | | | AAGGCCCAGATCAAGGAGTCCCTGAGGGCAGAGCTGAGGGTGACAGAG |
| | | | ATGTCTGGCCGGAGCGCCAATGCCGAGGCTGCCGCCAAGCAGGTGCAG |
| | | | CTGGTGGAGAGCGGAGGAGGAGTGGTGCAGCCCGGCAGAAGCCTGCGG |
| | | | CTGAGCTGTAAGGCCAGCGGCTACACCTTCACACGGTCCACCATGCACT |
| | | | GGGTGAGACAGGCCCCCGGACAGGGACTGGAGTGGATCGGCTATATCA |
| | | | ACCCTAGCTCCGCCTACACAAACTATAATCAGAAGTTTAAGGACCGGTT |
| | | | TACCATCAGCGCCGATAAGTCCAAGTCTACAGCCTTCCTGCAGATGGAC |
| | | | TCCCTGCGGCCAGAGGATACAGGCGTGTACTTCTGTGCCAGACCCCAGG |
| | | | TGCACTACGACTATAACGGCTTTCCCTATTGGGGCCAGGGCACCCCTGT |
| | | | GACAGTGTCTAGCGCTAGCACTAAGGGGCCTTCCGTGTTTCCACTGGCT |
| | | | CCCTCTAGTAAATCCACCTCTGGAGGCACAGCTGCACTGGGATGTCTGG |
| | | | TGAAGGATTACTTCCCTGAACCAGTCACAGTGAGTTGGAACTCAGGGGC |
| | | | TCTGACAAGTGGAGTCCATACTTTTCCCGCAGTGCTGCAGTCAAGCGGA |
| | | | CTGTACTCCCTGTCCTCTGTGGTCACCGTGCCTAGTTCAAGCCTGGGCAC |
| | | | CCAGACATATATCTGCAACGTGAATCACAAGCCATCAAATACAAAAGT |
| | | | CGACAAGAAAGTGGAGCCCAAGAGCTGTGATAAAACTCATACCTGCCC |
| | | | ACCTTGTCCGGCGCCAGAGGCTGCAGGAGGACCAAGCGTGTTCCTGTTT |
| | | | CCACCCAAGCCTAAAGACACACTGATGATTTCCCGAACCCCCGAAGTCA |
| | | | CATGCGTGGTCGTGTCTGTGAGTCACGAGGACCCTGAAGTCAAGTTCAA |
| | | | CTGGTACGTGGATGGCGTCGAGGTGCATAATGCCAAGACTAAACCTAG |
| | | | GGAGGAACAGTACAACTCAACCTATCGCGTCGTGAGCGTCCTGACAGT |
| | | | GCTGCACCAGGATTGGCTGAACGGCAAAGAATATAAGTGCAAAGTGAG |
| | | | CAATAAGGCCCTGCCCGCTCCTATCGAGAAAACCATTTCCAAGGCTAAA |
| | | | GGGCAGCCTCGCGAACCACAGGTCTACGTGTATCCTCCAAGCCGGGAC |
| | | | GAGCTGACAAAGAACCAGGTCTCCCTGACTTGTCTGGTGAAAGGGTTTT |
| | | | ACCCTAGTGATATCGCTGTGGAGTGGGAATCAAATGGACAGCCAGAGA |
| | | | ACAATTATAAGACTACCCCCCCTGTGCTGGACAGTGATGGGTCATTCGC |
| | | | ACTGGTCTCCAAGCTGACAGTGGACAAATCTCGGTGGCAGCAGGGAAA |
| | | | TGTCTTTTCATGTAGCGTGATGCATGAAGCACTGCACAACCATTACACC |
| | | | CAGAAGTCACTGTCACTGTCACCAGGA |
| 297 | 22086 | full nt | AATCCCCCTACCTTTTCTCCAGCCCTGCTGGTGGTGACAGAGGGCGACA |
| | | | ACGCCACCTTCACATGCTCTTTTAGCAACACCTCCGAGTCTTTCGTGCTG |
| | | | AATTGGTACAGAATGAGCCCATCCAACCAGACAGATAAGCTGGCCGCC |
| | | | TTTCCAGAGGACAGATCCCAGCCCGGCCAGGATTCTAGGTTCCGCGTGA |
| | | | CCCAGCTGCCCAATGGCAGGGACTTTCACATGAGCGTGGTGCGCGCCCG |
| | | | GAGAAACGATTCCGGCACATATCTGTGCGGAGCCATCTCTCTGGCCCCT |
| | | | AAGGCCCAGATCAAGGAGTCCCTGAGGGCAGAGCTGAGGGTGACAGAG |
| | | | GAGGCTGCCGCCAAGGAGGCTGCCGCCAAGATGTCTGGCCGGAGCGCC |
| | | | AATGCCCAGGTGCAGCTGGTGGAGAGCGGAGGAGGAGTGGTGCAGCCC |
| | | | GGCAGAAGCCTGCGGCTGAGCTGTAAGGCCAGCGGCTACACCTTCACA |
| | | | CGGTCCACCATGCACTGGGTGAGACAGGCCCCCGGACAGGGACTGGAG |
| | | | TGGATCGGCTATATCAACCCTAGCTCCGCCTACACAAACTATAATCAGA |
| | | | AGTTTAAGGACCGGTTTACCATCAGCGCCGATAAGTCCAAGTCTACAGC |
| | | | CTTCCTGCAGATGGACTCCCTGCGGCCAGAGGATACAGGCGTGTACTTC |
| | | | TGTGCCAGACCCCAGGTGCACTACGACTATAACGGCTTTCCCTATTGGG |
| | | | GCCAGGGCACCCCTGTGACAGTGTCTAGCGCTAGCACTAAGGGGCCTTC |
| | | | CGTGTTTCCACTGGCTCCCTCTAGTAAATCCACCTCTGGAGGCACAGCT |
| | | | GCACTGGGATGTCTGGTGAAGGATTACTTCCCTGAACCAGTCACAGTGA |
| | | | GTTGGAACTCAGGGGCTCTGACAAGTGGAGTCCATACTTTTCCCGCAGT |
| | | | GCTGCAGTCAAGCGGACTGTACTCCCTGTCCTCTGTGGTCACCGTGCCT |
| | | | AGTTCAAGCCTGGGCACCCAGACATATATCTGCAACGTGAATCACAAG |
| | | | CCATCAAATACAAAAGTCGACAAGAAAGTGGAGCCCAAGAGCTGTGAT |
| | | | AAAACTCATACCTGCCCACCTTGTCCGGCGCCAGAGGCTGCAGGAGGA |
| | | | CCAAGCGTGTTCCTGTTTCCACCCAAGCCTAAAGACACACTGATGATTT |
| | | | CCCGAACCCCCGAAGTCACATGCGTGGTCGTGTCTGTGAGTCACGAGGA |
| | | | CCCTGAAGTCAAGTTCAACTGGTACGTGGATGGCGTCGAGGTGCATAAT |
| | | | GCCAAGACTAAACCTAGGGAGGAACAGTACAACTCAACCTATCGCGTC |
| | | | GTGAGCGTCCTGACAGTGCTGCACCAGGATTGGCTGAACGGCAAAGAA |
| | | | TATAAGTGCAAAGTGAGCAATAAGGCCCTGCCCGCTCCTATCGAGAAA |
| | | | ACCATTTCCAAGGCTAAAGGGCAGCCTCGCGAACCACAGGTCTACGTGT |
| | | | ATCCTCCAAGCCGGGACGAGCTGACAAAGAACCAGGTCTCCCTGACTT |
| | | | GTCTGGTGAAAGGGTTTTACCCTAGTGATATCGCTGTGGAGTGGGAATC |
| | | | AAATGGACAGCCAGAGAACAATTATAAGACTACCCCCCCTGTGCTGGA |
| | | | CAGTGATGGGTCATTCGCACTGGTCTCCAAGCTGACAGTGGACAAATCT |
| | | | CGGTGGCAGCAGGGAAATGTCTTTTCATGTAGCGTGATGCATGAAGCAC |
| | | | TGCACAACCATTACACCCAGAAGTCACTGTCACTGTCACCAGGA |
| 298 | 22091 | full nt | GCCTTCACCGTGACAGTGCCAAAGGATCTGTACGTGGTGGAGTATGGCA |
| | | | GCAACATGACAATCGAGTGCAAGTTCCCAGTGGAGAAGCAGCTGGACC |
| | | | TGGCCGCCCTGATCGTGTACTGGGAGATGGAGGATAAGAATATCATCC |
| | | | AGTTTGTGCACGGCGAGGAGGACCTGAAGGTGCAGCACAGCTCCTATC |
| | | | GGCAGAGAGCCAGGCTGCTGAAGGATCAGCTGAGCCTGGGCAACGCCG |
| | | | CCCTGCAGATCACCGACGTGAAGCTGCAGGATGCCGGGGTGTACAGAT |
| | | | GCATGATCTCCTACGGCGGAGCCGACTATAAGCGGATCACCGTGAAGG |
| | | | TGAACGCCGAGGCTGCCGCCAAGGAGGCTGCCGCCAAGGATATCCAGA |
| | | | TGACACAGTCCCCTTCTAGCCTGTCTGCCAGCGTGGGCGACAGGGTGAC |
| | | | CATGACATGTTCCGCCTCCTCTAGCGTGTCTTACATGAACTGGTATCAG |
| | | | CAGAAGCCAGGCAAGGCCCCCAAGAGATGGATCTACGACTCCTCTAAG |
| | | | CTGGCCTCTGGCGTGCCCGCCAGGTTCTCCGGCTCTGGCAGCGGCACCG |
| | | | ATTATACCCTGACAATCAGCTCCCTGCAGCCTGAGGACTTCGCCACATA |
| | | | CTATTGTCAGCAGTGGTCCCGCAATCCCCCTACCTTTGGCGGCGGCACA |
| | | | AAGCTGCAGATCACCCGGACAGTGGCGGCGCCCAGTGTCTTCATTTTTC |
| | | | CCCCTAGCGACGAACAGCTGAAGTCTGGGACAGCCAGTGTGGTCTGTCT |
| | | | GCTGAACAACTTCTACCCTAGAGAGGCTAAAGTGCAGTGGAAGGTCGA |
| | | | TAACGCACTGCAGTCCGGAAATTCTCAGGAGAGTGTGACTGAACAGGA |
| | | | CTCAAAAGATAGCACCTATTCCCTGTCAAGCACACTGACTCTGAGCAAG |
| | | | GCCGACTACGAGAAGCATAAAGTGTATGCTTGTGAAGTCACCCACCAG |
| | | | GGGCTGAGTTCACCAGTCACAAAATCATTCAACAGAGGGGAGTGC |
| 299 | 22092 | full nt | GCCTTCACCGTGACAGTGCCAAAGGATCTGTACGTGGTGGAGTATGGCA |
| | | | GCAACATGACCATCGAGTGCAAGTTCCCAGTGGAGAAGCAGCTGGACC |
| | | | TGGCCGCCCTGCAGGTGTTCTGGATGATGGAGGATAAGAATATCATCCA |
| | | | GTTTGTGCACGGCGAGGAGGACCTGAAGGTGCAGCACAGCTCCTACCG |
| | | | GCAGAGAGCCAGGCTGCTGAAGGATCAGCTGTCTCTGGGCAACGCCGC |
| | | | CCTGCAGATCACCGACGTGAAGCTGCAGGATGCCGGGGTGTACACATG |
| | | | CCTGATCGCCTACAAGGGAGCCGACTATAAGCGGATCACCGTGAAGGT |
| | | | GAACGCCGAGGCTGCCGCCAAGGAGGCTGCCGCCAAGGATATCCAGAT |
| | | | GACACAGTCCCCTTCTAGCCTGTCTGCCAGCGTGGGCGACAGAGTGACC |
| | | | ATGACATGTTCCGCCTCCTCTAGCGTGTCTTACATGAACTGGTATCAGC |
| | | | AGAAGCCAGGCAAGGCCCCCAAGCGGTGGATCTACGACTCCTCTAAGC |
| | | | TGGCCAGCGGCGTGCCCGCCCGGTTTTCCGGCTCTGGCAGCGGCACCGA |
| | | | TTATACCCTGACAATCAGCTCCCTGCAGCCTGAGGACTTCGCCACATAC |
| | | | TATTGTCAGCAGTGGTCCAGAAATCCCCCTACCTTTGGCGGCGGCACAA |
| | | | AGCTGCAGATCACCAGGACAGTGGCGGCGCCCAGTGTCTTCATTTTTCC |
| | | | CCCTAGCGACGAACAGCTGAAGTCTGGGACAGCCAGTGTGGTCTGTCTG |
| | | | CTGAACAACTTCTACCCTAGAGAGGCTAAAGTGCAGTGGAAGGTCGAT |
| | | | AACGCACTGCAGTCCGGAAATTCTCAGGAGAGTGTGACTGAACAGGAC |
| | | | TCAAAAGATAGCACCTATTCCCTGTCAAGCACACTGACTCTGAGCAAGG |
| | | | CCGACTACGAGAAGCATAAAGTGTATGCTTGTGAAGTCACCCACCAGG |
| | | | GGCTGAGTTCACCAGTCACAAAATCATTCAACAGAGGGGAGTGC |
| 300 | 22094 | full nt | GCCTTCACCGTGACAGTGCCAAAGGATCTGTACGTGGTGGAGTATGGCA |
| | | | GCAACATGACAATCGAGTGCAAGTTCCCAGTGGAGAAGCAGCTGGACC |
| | | | TGGCCGCCCTGATCGTGTACTGGGAGATGGAGGATAAGAATATCATCC |
| | | | AGTTTGTGCACGGCGAGGAGGACCTGAAGGTGCAGCACAGCTCCTATC |
| | | | GGCAGAGAGCCAGGCTGCTGAAGGATCAGCTGTCTCTGGGCAACGCCG |
| | | | CCCTGCAGATCACCGACGTGAAGCTGCAGGATGCCGGGGTGTACAGAT |
| | | | GCATGATCAGCTACGGCGGAGCCGACTATAAGCGGATCACCGTGAAGG |
| | | | TGAACGCCGAGGCTGCCGCCAAGATGAGCGGCAGAAGCGCCAACGCCG |
| | | | ATATCCAGATGACACAGTCCCCTTCTAGCCTGTCTGCCAGCGTGGGCGA |
| | | | CAGGGTGACCATGACATGTAGCGCCTCCTCTAGCGTGTCCTACATGAAC |
| | | | TGGTATCAGCAGAAGCCAGGCAAGGCCCCCAAGAGATGGATCTACGAC |
| | | | TCCTCTAAGCTGGCCTCCGGCGTGCCCGCCAGGTTCTCCGGCTCTGGCA |
| | | | GCGGCACCGATTATACCCTGACAATCAGCTCCCTGCAGCCTGAGGACTT |
| | | | CGCCACATACTATTGTCAGCAGTGGTCTCGCAATCCCCCTACCTTTGGC |
| | | | GGCGGCACAAAGCTGCAGATCACCCGGACAGTGGCGGCGCCCAGTGTC |
| | | | TTCATTTTTCCCCCTAGCGACGAACAGCTGAAGTCTGGGACAGCCAGTG |
| | | | TGGTCTGTCTGCTGAACAACTTCTACCCTAGAGAGGCTAAAGTGCAGTG |
| | | | GAAGGTCGATAACGCACTGCAGTCCGGAAATTCTCAGGAGAGTGTGAC |
| | | | TGAACAGGACTCAAAAGATAGCACCTATTCCCTGTCAAGCACACTGACT |
| | | | CTGAGCAAGGCCGACTACGAGAAGCATAAAGTGTATGCTTGTGAAGTC |
| | | | ACCCACCAGGGGCTGAGTTCACCAGTCACAAAATCATTCAACAGAGGG |
| | | | GAGTGC |
| 301 | 22096 | full nt | GCCTTCACCGTGACAGTGCCAAAGGATCTGTACGTGGTGGAGTATGGCA |
| | | | GCAACATGACAATCGAGTGCAAGTTCCCAGTGGAGAAGCAGCTGGACC |
| | | | TGGCCGCCCTGATCGTGTACTGGGAGATGGAGGATAAGAATATCATCC |
| | | | AGTTTGTGCACGGCGAGGAGGACCTGAAGGTGCAGCACAGCTCCTATC |
| | | | GGCAGAGAGCCAGGCTGCTGAAGGATCAGCTGTCTCTGGGCAACGCCG |
| | | | CCCTGCAGATCACCGACGTGAAGCTGCAGGATGCCGGGGTGTACAGAT |
| | | | GCATGATCAGCTACGGCGGAGCCGACTATAAGCGGATCACAGTGAAGG |
| | | | TGAACGCCGAGGCTGCCGCCAAGGAGGCTGCCGCCAAGATGAGCGGCA |
| | | | GAAGCGCCAACGCCGATATCCAGATGACCCAGTCCCCTTCTAGCCTGTC |
| | | | TGCCAGCGTGGGCGACAGGGTGACCATGACATGTAGCGCCTCCTCTAGC |
| | | | GTGTCCTACATGAACTGGTATCAGCAGAAGCCAGGCAAGGCCCCCAAG |
| | | | AGATGGATCTACGACTCCTCTAAGCTGGCCTCCGGCGTGCCCGCCAGGT |
| | | | TCTCCGGCTCTGGCAGCGGCACCGATTATACCCTGACAATCAGCTCCCT |
| | | | GCAGCCTGAGGACTTCGCCACATACTATTGTCAGCAGTGGTCTCGCAAT |
| | | | CCCCCTACCTTTGGCGGCGGCACAAAGCTGCAGATCACCCGGACAGTG |
| | | | GCGGCGCCCAGTGTCTTCATTTTTCCCCCTAGCGACGAACAGCTGAAGT |
| | | | CTGGGACAGCCAGTGTGGTCTGTCTGCTGAACAACTTCTACCCTAGAGA |
| | | | GGCTAAAGTGCAGTGGAAGGTCGATAACGCACTGCAGTCCGGAAATTC |
| | | | TCAGGAGAGTGTGACTGAACAGGACTCAAAAGATAGCACCTATTCCCT |
| | | | GTCAAGCACACTGACTCTGAGCAAGGCCGACTACGAGAAGCATAAAGT |
| | | | GTATGCTTGTGAAGTCACCCACCAGGGGCTGAGTTCACCAGTCACAAAA |
| | | | TCATTCAACAGAGGGGAGTGC |
| 302 | 23571 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSGGGGSQVQLVESGGGVVQPGRSLRLSCAASGFTFSSYGMHWVRQA |
| | | | PGKGLEWVAFIRYDGSNKYYADSVKGRFTISRDNSKNTLYLQMNSLRAED |
| | | | TAVYYCKTHGSHDNWGQGTMVTVSSGGGGSGGGSGGGSGGGGSGGGGS |
| | | | QSVLTQPPSVSGAPGQRVTISCSGSRSNIGSNTVKWYQQLPGTAPKLLIYYN |
| | | | DQRPSGVPDRFSGSKSGTSASLAITGLQAEDEADYYCQSYDRYTHPALLFG |
| | | | TGTKVTVL |
| 303 | 24219 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSQVQLVESGGGVVQPGRSLRLSCAASGFTFSSYGMHWVRQAPGKGL |
| | | | EWVAFIRYDGSNKYYADSVKGRFTISRDNSKNTLYLQMNSLRAEDTAVYY |
| | | | CKTHGSHDNWGQGTMVTVSSGGGGSMSGRSANAGGGGSGGGGSQSVLT |
| | | | QPPSVSGAPGQRVTISCSGSRSNIGSNTVKWYQQLPGTAPKLLIYYNDQRPS |
| | | | GVPDRFSGSKSGTSASLAITGLQAEDEADYYCQSYDRYTHPALLFGTGTKV |
| | | | TVL |
| 304 | 24221 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSMSGRSANAGGGGSQ |
| | | | VQLVESGGGVVQPGRSLRLSCAASGFTFSSYGMHWVRQAPGKGLEWVAFI |
| | | | RYDGSNKYYADSVKGRFTISRDNSKNTLYLQMNSLRAEDTAVYYCKTHGS |
| | | | HDNWGQGTMVTVSSGGGGSMSGRSANAGGGGSGGGGSQSVLTQPPSVSG |
| | | | APGQRVTISCSGSRSNIGSNTVKWYQQLPGTAPKLLIYYNDQRPSGVPDRFS |
| | | | GSKSGTSASLAITGLQAEDEADYYCQSYDRYTHPALLFGTGTKVTVL |
| 305 | 24222 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGSMSGRSANAGGSQVQLV |
| | | | ESGGGVVQPGRSLRLSCAASGFTFSSYGMHWVRQAPGKGLEWVAFIRYDG |
| | | | SNKYYADSVKGRFTISRDNSKNTLYLQMNSLRAEDTAVYYCKTHGSHDN |
| | | | WGQGTMVTVSSGGGGSMSGRSANAGGGGSGGGGSQSVLTQPPSVSGAPG |
| | | | QRVTISCSGSRSNIGSNTVKWYQQLPGTAPKLLIYYNDQRPSGVPDRFSGSK |
| | | | SGTSASLAITGLQAEDEADYYCQSYDRYTHPALLFGTGTKVTVL |
| 306 | 24224 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSGGGGSQVQLVESGGGVVQPGRSLRLSCAASGFTFSSYGMHWVRQA |
| | | | PGKGLEWVAFIRYDGSNKYYADSVKGRFTISRDNSKNTLYLQMNSLRAED |
| | | | TAVYYCKTHGSHDNWGQGTMVTVSSGGSGGGSGGGSGGGSGGGSGQSV |
| | | | LTQPPSVSGAPGQRVTISCSGSRSNIGSNTVKWYQQLPGTAPKLLIYYNDQR |
| | | | PSGVPDRFSGSKSGTSASLAITGLQAEDEADYYCQSYDRYTHPALLFGTGT |
| | | | KVTVL |
| 307 | 24308 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSMSGRSANAGGGGSG |
| | | | GGGSQVQLVESGGGVVQPGRSLRLSCAASGFTFSSYGMHWVRQAPGKGL |
| | | | EWVAFIRYDGSNKYYADSVKGRFTISRDNSKNTLYLQMNSLRAEDTAVYY |
| | | | CKTHGSHDNWGQGTMVTVSSGGGGSMSGRSANAGGGGSGGGGSQSVLT |
| | | | QPPSVSGAPGQRVTISCSGSRSNIGSNTVKWYQQLPGTAPKLLIYYNDQRPS |
| | | | GVPDRFSGSKSGTSASLAITGLQAEDEADYYCQSYDRYTHPALLFGTGTKV |
| | | | TVL |
| 308 | 24831 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDVACPTAEETLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYNSSWSEWASVPCSGGGGSGGGGS |
| | | | GGGGSNLPVATPDPGMFPCLHHSQNLLRAVSNMLQKARQTLEFYPCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFYKTKIKLCILLHAFRIRAVTIDRVMSYLNAS |
| 309 | 24832 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYPCTSEEI |
| | | | DHEDITKDKTSTVEACLPLEATKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFYKTKIKLCILLHAFRIRAVTIDRVMSYLNAS |
| 310 | 24833 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYPCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFYKTKIKLCILLHAFAIRAVTIDRVMSYLNAS |
| 311 | 24834 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYPCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFYKTKIKLCILLHAFRIRAATIDRVMSYLNAS |
| 312 | 24835 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLRFYPCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPERQIFLDQNMLAVIDELMQALN |
| | | | FNSETVPQKSSLEEPDFYETKIKLCILLHAFRIRAVTIDRVMSYLNAS |
| 313 | 24836 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYSCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFYKTKQKLCSLLHAFRIRAVTIDRVMSYLNAS |
| 314 | 24837 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLESSPCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFSKTKIKLCILLHAFRIRAVTIDRVMSYLNAS |
| 315 | 24838 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYPCASRE |
| | | | SRHEDITKDKTSTVEACLPLELTKNESCLNSRETSFITNGSCLASRKTSFMM |
| | | | ALCLSSIYEDLKMYQVEFKTMNAKLLMDPKRQIFLDQNMLAVIDELMQAL |
| | | | NFNSETVPQKSSLEEPDFYKTKIKLCILLHAFRIRAVTIDRVMSYLNAS |
| 316 | 24839 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYPCASRK |
| | | | SRHKDITKDKTSTVEACLPLELTKNESCLNSRETSFITNGSCLASRKTSFMM |
| | | | ALCLSSIYEDLKMYQVEFKTMNAKLLMDPKRQIFLDQNMLAVIDELMQAL |
| | | | NFNSETVPQKSSLEEPDFYKTKIKLCILLHAFRIRAVTIDRVMSYLNAS |
| 317 | 24840 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLSQSSRVLGSGKTLTIQVSSFGDAG |
| | | | QYTCHKGGEVLSHSLLLLHKKEDGIWSTDILKDQKEPKNKTFLRCEAKNYS |
| | | | GRFTCWWLTTISTDLTFSVKSSRGSSDPQGVTCGAATLSAERVRGDNKEYE |
| | | | YSVECQEDSACPAAEESLPIEVMVDAVHDLKYENYTSSFFIRDIIKPDPPKN |
| | | | LQLKPLKNSRQVEVSWEYPDTWSTPHSYFSLTFCVQVQGKSKREKKDRVF |
| | | | TDKTSATVICRKNASISVRAQDRYYSSSWSEWASVPCSGGGGSGGGGSGG |
| | | | GGSNLPVATPDPGMFPCLHHSQNLLRAVSNMLQKARQTLEFYPCTSEEIDH |
| | | | EDITKDKTSTVEACLPLELTKNESCLNSRETSFITNGSCLASRKTSFMMALC |
| | | | LSSIYEDLKMYQVEFKTMNAKLLMDPKRQIFLDQNMLAVIDELMQALNFN |
| | | | SETVPQKSSLEEPDFYKTKIKLCILLHAFRIRAVTIDRVMSYLNAS |
| 318 | 24841 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQSSPKNKTFLRCEAKNY |
| | | | SGRFTCWWLTTISTDLTFSVKSSRGSSDPQGVTCGAATLSAERVSGDNKEY |
| | | | EYSVECQEDSACPAAEESLPIEVMVDAVHKLKYENYTSSFFIRDIIKPDPPK |
| | | | NLQLKPLKNSRQVEVSWEYPDTWSTPHSYFSLTFCVQVQGKSKREKKDRV |
| | | | FTDKTSATVICRKNASISVRAQDRYYSSSWSEWASVPCSGGGGSGGGGSG |
| | | | GGGSNLPVATPDPGMFPCLHHSQNLLRAVSNMLQKARQTLEFYPCTSEEID |
| | | | HEDITKDKTSTVEACLPLELTKNESCLNSRETSFITNGSCLASRKTSFMMAL |
| | | | CLSSIYEDLKMYQVEFKTMNAKLLMDPKRQIFLDQNMLAVIDELMQALNF |
| | | | NSETVPQKSSLEEPDFYKTKIKLCILLHAFRIRAVTIDRVMSYLNAS |
| 319 | 24842 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPISVMVDAVHKLKYENYSSRFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYPCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFYKTKIKLCILLHAFRIRAVTIDRVMSYLNAS |
| 320 | 26498 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSMSGRSANAG |
| | | | GGGSGGGGSQVQLVESGGGVVQPGRSLRLSCAASGVTFSSYGMHWVRQA |
| | | | PGKGLEWVAFIRYDGSNKYYADSVKGRFTISRDNSKNTLYLQMNSLRAED |
| | | | TAVYYCKTHGSHDNWGQGTMVTVSSGGSGGGSGGGSGGGSGGGSGQSV |
| | | | LTQPPSVSGAPGQRVTISCSGSRSNIGSNTVKWYQQLPGTAPKLLIYYNDQR |
| | | | PSGVPDRFSGSKSGTSASLAITGLQAEDEADYYCQSYDRYTHPALLFGTGT |
| | | | KVTVL |
| 321 | 23571 | full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGAGGAGGCTCTGGAGGAGGAGGCTCCATGA |
| | | | GCGGGCGGAGCGCCAACGCAGGGGGCGGCGGCTCTGGCGGCGGCGGC |
| | | | AGCCAGGTGCAGCTGGTGGAGAGCGGCGGCGGCGTGGTGCAGCCCGGC |
| | | | AGAAGCCTGCGGCTGAGCTGCGCAGCCTCTGGCTTCACCTTTAGCTCCT |
| | | | ACGGCATGCACTGGGTGCGGCAGGCCCCTGGCAAGGGACTGGAGTGGG |
| | | | TGGCCTTCATCAGATATGACGGCTCCAATAAGTACTATGCCGATTCTGT |
| | | | GAAGGGCAGGTTTACCATCAGCCGCGACAACTCCAAGAATACACTGTA |
| | | | CCTGCAGATGAACTCCCTGAGGGCCGAGGACACCGCCGTGTACTATTGC |
| | | | AAGACACACGGCTCTCACGATAATTGGGGCCAGGGCACCATGGTGACA |
| | | | GTGTCTTCCGGAGGAGGAGGCAGCGGCGGCGGGAGCGGCGGCGGCAGC |
| | | | GGGGGTGGAGGCTCCGGAGGAGGAGGCTCTCAGAGCGTGCTGACCCAG |
| | | | CCACCTTCCGTGTCTGGAGCCCCCGGACAGAGGGTGACAATCAGCTGTT |
| | | | CCGGCTCTCGCAGCAACATCGGCAGCAATACCGTGAAGTGGTATCAGC |
| | | | AGCTGCCAGGCACAGCCCCCAAGCTGCTGATCTACTATAATGACCAGCG |
| | | | GCCTTCCGGCGTGCCAGATAGATTCTCCGGCTCTAAGAGCGGCACCTCC |
| | | | GCCTCTCTGGCCATCACAGGCCTGCAGGCAGAGGACGAGGCAGATTAC |
| | | | TATTGTCAGTCCTACGATAGATATACCCACCCCGCCCTGCTGTTTGGCA |
| | | | CCGGCACAAAGGTGACAGTGCTG |
| 322 | 24219 | full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGAGGAGGCTCTGGAGGAGGAGGCTCCATGA |
| | | | GCGGGCGGAGCGCCAACGCAGGGGGCGGCGGCTCTCAGGTGCAGCTGG |
| | | | TGGAGAGCGGAGGAGGAGTGGTGCAGCCCGGCAGAAGCCTGCGGCTGA |
| | | | GCTGCGCAGCCTCTGGCTTCACCTTTAGCTCCTACGGCATGCACTGGGT |
| | | | GCGGCAGGCCCCTGGCAAGGGACTGGAGTGGGTGGCCTTCATCAGATA |
| | | | TGACGGCTCCAATAAGTACTATGCCGATTCTGTGAAGGGCAGGTTTACC |
| | | | ATCAGCCGCGACAACTCCAAGAATACACTGTACCTGCAGATGAACTCCC |
| | | | TGAGGGCCGAGGACACCGCCGTGTACTATTGCAAGACACACGGCTCTC |
| | | | ACGATAATTGGGGCCAGGGCACCATGGTGACAGTGTCTTCCGGAGGAG |
| | | | GAGGCAGCATGAGCGGGCGGAGCGCCAACGCAGGGGGTGGAGGCTCC |
| | | | GGAGGAGGAGGCTCTCAGAGCGTGCTGACCCAGCCACCTTCCGTGTCTG |
| | | | GAGCCCCCGGACAGAGGGTGACAATCAGCTGTTCCGGCTCTCGCAGCA |
| | | | ACATCGGCAGCAATACCGTGAAGTGGTATCAGCAGCTGCCAGGCACAG |
| | | | CCCCCAAGCTGCTGATCTACTATAATGACCAGCGGCCTTCCGGCGTGCC |
| | | | AGATAGATTCTCCGGCTCTAAGAGCGGCACCTCCGCCTCTCTGGCCATC |
| | | | ACAGGCCTGCAGGCAGAGGACGAGGCAGATTACTATTGTCAGAGCTAC |
| | | | GATAGATATACCCACCCCGCCCTGCTGTTTGGCACCGGCACAAAGGTGA |
| | | | CAGTGCTG |
| 323 | 24221 | full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGAGGAGGCTCCATGAGCGGGCGGAGCGCCA |
| | | | ACGCAGGGGGCGGCGGCTCTCAGGTGCAGCTGGTGGAGAGCGGAGGAG |
| | | | GAGTGGTGCAGCCCGGCAGAAGCCTGCGGCTGAGCTGCGCAGCCTCTG |
| | | | GCTTCACCTTTAGCTCCTACGGCATGCACTGGGTGCGGCAGGCCCCTGG |
| | | | CAAGGGACTGGAGTGGGTGGCCTTCATCAGATATGACGGCTCCAATAA |
| | | | GTACTATGCCGATTCTGTGAAGGGCAGGTTTACCATCAGCCGCGACAAC |
| | | | TCCAAGAATACACTGTACCTGCAGATGAACTCTCTGAGGGCCGAGGAC |
| | | | ACCGCCGTGTACTATTGCAAGACACACGGCTCCCACGATAATTGGGGCC |
| | | | AGGGCACCATGGTGACAGTGTCTTCCGGAGGAGGAGGCAGCATGAGCG |
| | | | GGCGGAGCGCCAACGCAGGGGGTGGAGGCTCCGGAGGAGGAGGCTCTC |
| | | | AGAGCGTGCTGACCCAGCCACCTTCCGTGTCTGGAGCCCCCGGACAGA |
| | | | GGGTGACAATCAGCTGTTCCGGCTCTCGCAGCAACATCGGCAGCAATAC |
| | | | CGTGAAGTGGTATCAGCAGCTGCCAGGCACAGCCCCCAAGCTGCTGAT |
| | | | CTACTATAATGACCAGCGGCCTTCCGGCGTGCCAGATAGATTCTCCGGC |
| | | | TCTAAGAGCGGCACCTCCGCCTCTCTGGCCATCACAGGCCTGCAGGCAG |
| | | | AGGACGAGGCAGATTACTATTGTCAGAGCTACGATAGATATACCCACC |
| | | | CCGCCCTGCTGTTTGGCACCGGCACAAAGGTGACAGTGCTG |
| 324 | 24222 | full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGCTCCATGAGCGGGCGGAGCGCCAACGCAG |
| | | | GGGGCAGCCAGGTGCAGCTGGTGGAGAGCGGAGGAGGAGTGGTGCAG |
| | | | CCCGGCAGAAGCCTGCGGCTGAGCTGCGCAGCCTCTGGCTTCACCTTTA |
| | | | GCTCCTACGGCATGCACTGGGTGCGGCAGGCCCCTGGCAAGGGACTGG |
| | | | AGTGGGTGGCCTTCATCAGATATGACGGCTCCAATAAGTACTATGCCGA |
| | | | TTCTGTGAAGGGCAGGTTTACCATCAGCCGCGACAACTCCAAGAATACA |
| | | | CTGTACCTGCAGATGAACTCTCTGAGGGCCGAGGACACCGCCGTGTACT |
| | | | ATTGCAAGACACACGGCTCCCACGATAATTGGGGCCAGGGCACCATGG |
| | | | TGACAGTGTCTAGCGGAGGAGGAGGCAGCATGAGCGGGCGGAGCGCCA |
| | | | ACGCAGGGGGTGGAGGCTCCGGAGGAGGAGGCTCTCAGAGCGTGCTGA |
| | | | CCCAGCCACCTTCCGTGTCTGGAGCCCCCGGACAGAGGGTGACAATCA |
| | | | GCTGTTCCGGCTCTCGCAGCAACATCGGCAGCAATACCGTGAAGTGGTA |
| | | | TCAGCAGCTGCCAGGCACAGCCCCCAAGCTGCTGATCTACTATAATGAC |
| | | | CAGCGGCCTTCCGGCGTGCCAGATAGATTCTCCGGCTCTAAGAGCGGCA |
| | | | CCTCCGCCTCTCTGGCCATCACAGGCCTGCAGGCAGAGGACGAGGCAG |
| | | | ATTACTATTGTCAGAGCTACGATAGATATACCCACCCCGCCCTGCTGTT |
| | | | TGGCACCGGCACAAAGGTGACAGTGCTG |
| 325 | 24224 | full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGAGGAGGCTCTGGAGGAGGAGGCTCCATGA |
| | | | GCGGGCGGAGCGCCAACGCAGGGGGCGGCGGCTCCGGCGGCGGCGGCT |
| | | | CTCAGGTGCAGCTGGTGGAGAGCGGAGGAGGAGTGGTGCAGCCCGGCA |
| | | | GAAGCCTGCGGCTGAGCTGCGCAGCCAGCGGCTTCACCTTTAGCTCCTA |
| | | | CGGCATGCACTGGGTGCGGCAGGCCCCTGGCAAGGGACTGGAGTGGGT |
| | | | GGCCTTCATCAGATATGACGGCAGCAATAAGTACTATGCCGATTCCGTG |
| | | | AAGGGCAGGTTTACCATCTCCCGCGACAACTCTAAGAATACACTGTACC |
| | | | TGCAGATGAACTCCCTGCGCGCAGAGGACACCGCCGTGTACTATTGCAA |
| | | | GACACACGGCTCTCACGATAATTGGGGCCAGGGCACCATGGTGACAGT |
| | | | GTCTAGCGGAGGCAGCGGAGGAGGCTCCGGAGGAGGCTCTGGCGGCGG |
| | | | CAGCGGCGGCGGCTCTGGACAGAGCGTGCTGACCCAGCCACCTAGCGT |
| | | | GTCCGGAGCCCCCGGCCAGAGGGTGACAATCTCTTGTAGCGGCTCCCGC |
| | | | TCTAACATCGGCAGCAATACCGTGAAGTGGTATCAGCAGCTGCCAGGC |
| | | | ACAGCCCCCAAGCTGCTGATCTACTATAACGACCAGCGGCCTTCCGGCG |
| | | | TGCCAGATAGATTCAGCGGCTCCAAGTCTGGCACCAGCGCCTCCCTGGC |
| | | | CATCACAGGCCTGCAGGCAGAGGACGAGGCAGATTACTATTGTCAGTC |
| | | | CTACGATCGGTATACCCACCCCGCCCTGCTGTTTGGCACCGGCACAAAG |
| | | | GTGACAGTGCTG |
| 326 | 24308 | full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGCGGCGGCGGCGGCTCCATGAGCGGGCGGAGCGCCA |
| | | | ACGCAGGGGGAGGAGGCTCTGGAGGAGGAGGCAGCCAGGTGCAGCTG |
| | | | GTGGAGTCTGGAGGAGGAGTGGTGCAGCCCGGCAGAAGCCTGCGGCTG |
| | | | AGCTGCGCAGCCTCTGGCTTCACCTTTAGCTCCTACGGCATGCACTGGG |
| | | | TGCGGCAGGCCCCTGGCAAGGGACTGGAGTGGGTGGCCTTCATCAGAT |
| | | | ATGACGGCTCCAATAAGTACTATGCCGATTCTGTGAAGGGCAGGTTTAC |
| | | | CATCAGCCGCGACAACTCCAAGAATACACTGTACCTGCAGATGAACTCT |
| | | | CTGAGGGCCGAGGACACCGCCGTGTACTATTGCAAGACACACGGCAGC |
| | | | CACGATAATTGGGGCCAGGGCACCATGGTGACAGTGTCTTCCGGAGGA |
| | | | GGAGGCAGCATGAGCGGGCGGAGCGCCAACGCAGGGGGTGGAGGCTC |
| | | | CGGAGGAGGAGGCTCTCAGAGCGTGCTGACCCAGCCACCTTCCGTGTCT |
| | | | GGAGCCCCCGGACAGAGGGTGACAATCAGCTGTTCCGGCTCTCGCAGC |
| | | | AACATCGGCAGCAATACCGTGAAGTGGTATCAGCAGCTGCCAGGCACA |
| | | | GCCCCCAAGCTGCTGATCTACTATAATGACCAGCGGCCTTCCGGCGTGC |
| | | | CAGATAGATTCTCCGGCTCTAAGAGCGGCACCTCCGCCTCTCTGGCCAT |
| | | | CACAGGCCTGCAGGCAGAGGACGAGGCAGATTACTATTGTCAGAGCTA |
| | | | CGATAGATATACCCACCCCGCCCTGCTGTTTGGCACCGGCACAAAGGTG |
| | | | ACAGTGCTG |
| 327 | 24831 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACGTGGCCTGTCCCACCGCCGAGGAG |
| | | | ACCCTGCCTATCGAAGTGATGGTGGATGCCGTGCACAAGCTGAAGTAC |
| | | | GAGAATTATACAAGCTCCTTCTTTATCAGGGACATCATCAAGCCCGATC |
| | | | CCCCTAAGAACCTGCAGCTGAAGCCTCTGAAGAATAGCCGCCAGGTGG |
| | | | AGGTGTCCTGGGAGTACCCTGACACCTGGAGCACACCACACTCCTATTT |
| | | | CTCTCTGACCTTTTGCGTGCAGGTGCAGGGCAAGTCTAAGAGGGAGAA |
| | | | GAAGGACCGCGTGTTCACCGATAAGACAAGCGCCACCGTGATCTGTCG |
| | | | GAAGAACGCCAGCATCTCCGTGCGGGCCCAGGATAGATACTATAACAG |
| | | | CTCCTGGAGCGAGTGGGCCTCCGTGCCATGTTCTGGAGGAGGAGGCAG |
| | | | CGGCGGAGGAGGCTCCGGCGGCGGCGGCTCTAATCTGCCAGTGGCCAC |
| | | | CCCTGACCCAGGCATGTTCCCCTGCCTGCACCACTCTCAGAACCTGCTG |
| | | | CGGGCCGTGAGCAATATGCTGCAGAAGGCCAGACAGACACTGGAGTTT |
| | | | TACCCATGTACCTCCGAGGAGATCGACCACGAGGATATCACAAAGGAT |
| | | | AAGACCTCTACAGTGGAGGCATGCCTGCCACTGGAGCTGACCAAGAAC |
| | | | GAGAGCTGTCTGAACAGCCGGGAGACATCTTTCATCACCAACGGCAGC |
| | | | TGCCTGGCCTCCAGAAAGACATCTTTTATGATGGCCCTGTGCCTGTCTA |
| | | | GCATCTACGAGGACCTGAAGATGTATCAGGTGGAGTTCAAGACCATGA |
| | | | ACGCCAAGCTGCTGATGGACCCTAAGAGGCAGATCTTTCTGGATCAGA |
| | | | ATATGCTGGCCGTGATCGACGAGCTGATGCAGGCCCTGAACTTCAATAG |
| | | | CGAGACAGTGCCACAGAAGTCCTCTCTGGAGGAGCCCGATTTCTACAA |
| | | | GACCAAGATCAAGCTGTGCATCCTGCTGCACGCCTTCCGGATCAGAGCC |
| | | | GTGACCATCGACCGCGTGATGAGCTACCTGAACGCCAGC |
| 328 | 24832 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAAGTCTAAGAGGGAGAAG |
| | | | AAGGACCGCGTGTTCACCGATAAGACAAGCGCCACCGTGATCTGTCGG |
| | | | AAGAACGCCAGCATCTCCGTGCGGGCCCAGGATAGATACTATTCTAGCT |
| | | | CCTGGAGCGAGTGGGCCTCCGTGCCATGTTCTGGAGGAGGAGGCAGCG |
| | | | GCGGAGGAGGCTCCGGCGGCGGCGGCTCTAATCTGCCAGTGGCCACCC |
| | | | CTGACCCAGGCATGTTCCCCTGCCTGCACCACTCTCAGAACCTGCTGCG |
| | | | GGCCGTGAGCAATATGCTGCAGAAGGCCAGACAGACACTGGAGTTTTA |
| | | | CCCATGTACCTCCGAGGAGATCGACCACGAGGATATCACAAAGGATAA |
| | | | GACCTCTACAGTGGAGGCATGCCTGCCACTGGAGGCCACCAAGAACGA |
| | | | GAGCTGTCTGAACAGCCGGGAGACATCTTTCATCACCAACGGCAGCTGC |
| | | | CTGGCCTCCAGAAAGACATCTTTTATGATGGCCCTGTGCCTGTCTAGCA |
| | | | TCTACGAGGACCTGAAGATGTATCAGGTGGAGTTCAAGACCATGAACG |
| | | | CCAAGCTGCTGATGGACCCTAAGAGGCAGATCTTTCTGGATCAGAATAT |
| | | | GCTGGCCGTGATCGACGAGCTGATGCAGGCCCTGAACTTCAATAGCGA |
| | | | GACAGTGCCACAGAAGTCCTCTCTGGAGGAGCCCGATTTCTACAAGACC |
| | | | AAGATCAAGCTGTGCATCCTGCTGCACGCCTTCCGGATCAGAGCCGTGA |
| | | | CCATCGACCGCGTGATGAGCTACCTGAACGCCAGC |
| 329 | 24833 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAAGTCTAAGAGGGAGAAG |
| | | | AAGGACCGCGTGTTCACCGATAAGACAAGCGCCACCGTGATCTGTCGG |
| | | | AAGAACGCCAGCATCTCCGTGCGGGCCCAGGATAGATACTATTCTAGCT |
| | | | CCTGGAGCGAGTGGGCCTCCGTGCCATGTTCTGGAGGAGGAGGCAGCG |
| | | | GCGGAGGAGGCTCCGGCGGCGGCGGCTCTAATCTGCCAGTGGCCACCC |
| | | | CTGACCCAGGCATGTTCCCCTGCCTGCACCACTCTCAGAACCTGCTGCG |
| | | | GGCCGTGAGCAATATGCTGCAGAAGGCCAGACAGACACTGGAGTTTTA |
| | | | CCCATGTACCTCCGAGGAGATCGACCACGAGGATATCACAAAGGATAA |
| | | | GACCTCTACAGTGGAGGCATGCCTGCCACTGGAGCTGACCAAGAACGA |
| | | | GAGCTGTCTGAACAGCCGGGAGACATCTTTCATCACCAACGGCAGCTGC |
| | | | CTGGCCTCCAGAAAGACATCTTTTATGATGGCCCTGTGCCTGTCTAGCA |
| | | | TCTACGAGGACCTGAAGATGTATCAGGTGGAGTTCAAGACCATGAACG |
| | | | CCAAGCTGCTGATGGACCCTAAGAGGCAGATCTTTCTGGATCAGAATAT |
| | | | GCTGGCCGTGATCGACGAGCTGATGCAGGCCCTGAACTTCAATAGCGA |
| | | | GACAGTGCCACAGAAGTCCTCTCTGGAGGAGCCCGATTTCTACAAGACC |
| | | | AAGATCAAGCTGTGCATCCTGCTGCACGCCTTCGCCATCAGAGCCGTGA |
| | | | CCATCGACCGCGTGATGAGCTACCTGAACGCCAGC |
| 330 | 24834 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAAGTCTAAGAGGGAGAAG |
| | | | AAGGACCGCGTGTTCACCGATAAGACAAGCGCCACCGTGATCTGTCGG |
| | | | AAGAACGCCAGCATCTCCGTGCGGGCCCAGGATAGATACTATTCTAGCT |
| | | | CCTGGAGCGAGTGGGCCTCCGTGCCATGTTCTGGAGGAGGAGGCAGCG |
| | | | GCGGAGGAGGCTCCGGCGGCGGCGGCTCTAATCTGCCAGTGGCCACCC |
| | | | CTGACCCAGGCATGTTCCCCTGCCTGCACCACTCTCAGAACCTGCTGCG |
| | | | GGCCGTGAGCAATATGCTGCAGAAGGCCAGACAGACACTGGAGTTTTA |
| | | | CCCATGTACCTCCGAGGAGATCGACCACGAGGATATCACAAAGGATAA |
| | | | GACCTCTACAGTGGAGGCATGCCTGCCACTGGAGCTGACCAAGAACGA |
| | | | GAGCTGTCTGAACAGCCGGGAGACATCTTTCATCACCAACGGCAGCTGC |
| | | | CTGGCCTCCAGAAAGACATCTTTTATGATGGCCCTGTGCCTGTCTAGCA |
| | | | TCTACGAGGACCTGAAGATGTATCAGGTGGAGTTCAAGACCATGAACG |
| | | | CCAAGCTGCTGATGGACCCTAAGAGGCAGATCTTTCTGGATCAGAATAT |
| | | | GCTGGCCGTGATCGACGAGCTGATGCAGGCCCTGAACTTCAATAGCGA |
| | | | GACAGTGCCACAGAAGTCCTCTCTGGAGGAGCCCGATTTCTACAAGACC |
| | | | AAGATCAAGCTGTGCATCCTGCTGCACGCCTTCCGGATCAGAGCCGCCA |
| | | | CCATCGACCGCGTGATGAGCTACCTGAACGCCAGC |
| 331 | 24835 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCACAGGTGTACGTGTATCCCCCTTCCCGGGACGAGCTGACCAAGA |
| | | | ACCAGGTGTCTCTGACATGCCTGGTGAAGGGCTTCTACCCCAGCGATAT |
| | | | CGCCGTGGAGTGGGAGTCCAATGGCCAGCCTGAGAACAATTATAAGAC |
| | | | CACACCACCCGTGCTGGACAGCGATGGCTCCTTCGCCCTGGTGTCCAAG |
| | | | CTGACCGTGGACAAGTCTAGGTGGCAGCAGGGCAACGTGTTTTCTTGCA |
| | | | GCGTGATGCACGAGGCCCTGCACAATCACTACACCCAGAAGTCCCTGA |
| | | | GCTTAAGCCCAGGAGGCAGCGCCGATGGAGGAATCTGGGAGCTGAAGA |
| | | | AGGACGTGTACGTGGTGGAGCTGGACTGGTACCCGGATGCCCCAGGCG |
| | | | AGATGGTGGTGCTGACCTGCGACACACCTGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACATGTCACA |
| | | | AGGGAGGAGAGGTGCTGAGCCACTCCCTGCTGCTGCTGCACAAGAAGG |
| | | | AGGACGGCATCTGGAGCACAGACATCCTGAAGGATCAGAAGGAGCCAA |
| | | | AGAACAAGACCTTCCTGCGGTGCGAGGCCAAGAATTATAGCGGCAGAT |
| | | | TCACCTGTTGGTGGCTGACCACAATCAGCACCGATCTGACATTTTCCGT |
| | | | GAAGTCTAGCAGGGGCTCCTCTGACCCCCAGGGAGTGACATGCGGAGC |
| | | | CGCCACCCTGAGCGCCGAGCGGGTGAGAGGCGATAACAAGGAGTACGA |
| | | | GTATTCCGTGGAGTGCCAGGAGGACTCTGCCTGTCCAGCCGCCGAGGA |
| | | | GTCCCTGCCAATCGAAGTGATGGTGGATGCCGTGCACAAGCTGAAGTA |
| | | | CGAGAATTATACAAGCTCCTTCTTTATCAGGGACATCATCAAGCCCGAT |
| | | | CCCCCTAAGAACCTGCAGCTGAAGCCCCTGAAGAACAGCAGACAGGTG |
| | | | GAGGTGAGCTGGGAGTACCCCGACACCTGGAGCACACCTCACTCCTATT |
| | | | TCTCTCTGACCTTTTGCGTGCAGGTGCAGGGCAAGTCTAAGAGGGAGAA |
| | | | GAAGGACCGCGTGTTCACCGATAAGACAAGCGCCACCGTGATCTGTAG |
| | | | AAAGAACGCCTCTATCAGCGTGCGGGCACAGGACCGGTACTACAGCTC |
| | | | CTCTTGGTCCGAGTGGGCCTCTGTGCCATGCAGCGGAGGAGGAGGCTCC |
| | | | GGAGGAGGAGGCTCTGGCGGCGGCGGCAGCAACCTGCCTGTGGCCACC |
| | | | CCCGATCCTGGCATGTTCCCATGCCTGCACCACAGCCAGAACCTGCTGC |
| | | | GGGCCGTGTCCAATATGCTGCAGAAGGCCAGGCAGACCCTGCGCTTTTA |
| | | | TCCCTGTACATCTGAGGAGATCGACCACGAGGATATCACCAAGGACAA |
| | | | GACCAGCACAGTGGAGGCCTGCCTGCCTCTGGAGCTGACAAAGAACGA |
| | | | GTCCTGTCTGAACAGCCGGGAGACCAGCTTCATCACAAATGGCTCCTGC |
| | | | CTGGCCTCTAGAAAGACCAGCTTTATGATGGCCCTGTGCCTGAGCTCCA |
| | | | TCTACGAGGATCTGAAGATGTATCAGGTGGAGTTCAAGACAATGAACG |
| | | | CCAAGCTGCTGATGGACCCCGAGAGGCAGATCTTTCTGGATCAGAATAT |
| | | | GCTGGCCGTGATCGACGAGCTGATGCAGGCCCTGAACTTCAATTCCGAG |
| | | | ACCGTGCCACAGAAGTCTAGCCTGGAGGAGCCCGATTTCTACGAGACA |
| | | | AAGATCAAGCTGTGCATCCTGCTGCACGCCTTTCGGATCAGAGCCGTGA |
| | | | CAATCGACCGCGTGATGTCCTATCTGAACGCCTCT |
| 332 | 24836 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAAGTCTAAGAGGGAGAAG |
| | | | AAGGACCGCGTGTTCACCGATAAGACAAGCGCCACCGTGATCTGTCGG |
| | | | AAGAACGCCAGCATCTCCGTGCGGGCCCAGGATAGATACTATTCTAGCT |
| | | | CCTGGAGCGAGTGGGCCTCCGTGCCATGTTCTGGAGGAGGAGGCAGCG |
| | | | GCGGAGGAGGCTCCGGCGGCGGCGGCTCTAATCTGCCAGTGGCCACCC |
| | | | CTGACCCAGGCATGTTCCCCTGCCTGCACCACTCTCAGAACCTGCTGCG |
| | | | GGCCGTGAGCAATATGCTGCAGAAGGCCAGACAGACACTGGAGTTTTA |
| | | | CAGCTGTACCTCCGAGGAGATCGACCACGAGGATATCACAAAGGATAA |
| | | | GACCTCTACAGTGGAGGCATGCCTGCCACTGGAGCTGACCAAGAACGA |
| | | | GAGCTGTCTGAACAGCCGGGAGACATCTTTCATCACCAACGGCAGCTGC |
| | | | CTGGCCTCCAGAAAGACATCTTTTATGATGGCCCTGTGCCTGTCTAGCA |
| | | | TCTACGAGGACCTGAAGATGTATCAGGTGGAGTTCAAGACCATGAACG |
| | | | CCAAGCTGCTGATGGACCCTAAGAGGCAGATCTTTCTGGATCAGAATAT |
| | | | GCTGGCCGTGATCGACGAGCTGATGCAGGCCCTGAACTTCAATAGCGA |
| | | | GACAGTGCCACAGAAGTCCTCTCTGGAGGAGCCCGATTTCTACAAGACC |
| | | | AAGCAGAAGCTGTGCAGCCTGCTGCACGCCTTCCGGATCAGAGCCGTG |
| | | | ACCATCGACCGCGTGATGAGCTACCTGAACGCCAGC |
| 333 | 24837 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAAGTCTAAGAGGGAGAAG |
| | | | AAGGACCGCGTGTTCACCGATAAGACAAGCGCCACCGTGATCTGTCGG |
| | | | AAGAACGCCAGCATCTCCGTGCGGGCCCAGGATAGATACTATTCTAGCT |
| | | | CCTGGAGCGAGTGGGCCTCCGTGCCATGTTCTGGAGGAGGAGGCAGCG |
| | | | GCGGAGGAGGCTCCGGCGGCGGCGGCTCTAATCTGCCAGTGGCCACCC |
| | | | CTGACCCAGGCATGTTCCCCTGCCTGCACCACTCTCAGAACCTGCTGCG |
| | | | GGCCGTGAGCAATATGCTGCAGAAGGCCAGACAGACACTGGAGAGCAG |
| | | | CCCATGTACCTCCGAGGAGATCGACCACGAGGATATCACAAAGGATAA |
| | | | GACCTCTACAGTGGAGGCATGCCTGCCACTGGAGCTGACCAAGAACGA |
| | | | GAGCTGTCTGAACAGCCGGGAGACATCTTTCATCACCAACGGCAGCTGC |
| | | | CTGGCCTCCAGAAAGACATCTTTTATGATGGCCCTGTGCCTGTCTAGCA |
| | | | TCTACGAGGACCTGAAGATGTATCAGGTGGAGTTCAAGACCATGAACG |
| | | | CCAAGCTGCTGATGGACCCTAAGAGGCAGATCTTTCTGGATCAGAATAT |
| | | | GCTGGCCGTGATCGACGAGCTGATGCAGGCCCTGAACTTCAATAGCGA |
| | | | GACAGTGCCACAGAAGTCCTCTCTGGAGGAGCCCGATTTCAGCAAGAC |
| | | | CAAGATCAAGCTGTGCATCCTGCTGCACGCCTTCCGGATCAGAGCCGTG |
| | | | ACCATCGACCGCGTGATGAGCTACCTGAACGCCAGC |
| 334 | 24838 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAAGTCTAAGAGGGAGAAG |
| | | | AAGGACCGCGTGTTCACCGATAAGACAAGCGCCACCGTGATCTGTCGG |
| | | | AAGAACGCCAGCATCTCCGTGCGGGCCCAGGATAGATACTATTCTAGCT |
| | | | CCTGGAGCGAGTGGGCCTCCGTGCCATGTTCTGGAGGAGGAGGCAGCG |
| | | | GCGGAGGAGGCTCCGGCGGCGGCGGCTCTAATCTGCCAGTGGCCACCC |
| | | | CTGACCCAGGCATGTTCCCCTGCCTGCACCACTCTCAGAACCTGCTGCG |
| | | | GGCCGTGAGCAATATGCTGCAGAAGGCCAGACAGACACTGGAGTTTTA |
| | | | CCCATGTGCCTCCAGAGAGAGCAGACACGAGGATATCACAAAGGATAA |
| | | | GACCTCTACAGTGGAGGCATGCCTGCCACTGGAGCTGACCAAGAACGA |
| | | | GAGCTGTCTGAACAGCCGGGAGACATCTTTCATCACCAACGGCAGCTGC |
| | | | CTGGCCTCCAGAAAGACATCTTTTATGATGGCCCTGTGCCTGTCTAGCA |
| | | | TCTACGAGGACCTGAAGATGTATCAGGTGGAGTTCAAGACCATGAACG |
| | | | CCAAGCTGCTGATGGACCCTAAGAGGCAGATCTTTCTGGATCAGAATAT |
| | | | GCTGGCCGTGATCGACGAGCTGATGCAGGCCCTGAACTTCAATAGCGA |
| | | | GACAGTGCCACAGAAGTCCTCTCTGGAGGAGCCCGATTTCTACAAGACC |
| | | | AAGATCAAGCTGTGCATCCTGCTGCACGCCTTCCGGATCAGAGCCGTGA |
| | | | CCATCGACCGCGTGATGAGCTACCTGAACGCCAGC |
| 335 | 24839 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAAGTCTAAGAGGGAGAAG |
| | | | AAGGACCGCGTGTTCACCGATAAGACAAGCGCCACCGTGATCTGTCGG |
| | | | AAGAACGCCAGCATCTCCGTGCGGGCCCAGGATAGATACTATTCTAGCT |
| | | | CCTGGAGCGAGTGGGCCTCCGTGCCATGTTCTGGAGGAGGAGGCAGCG |
| | | | GCGGAGGAGGCTCCGGCGGCGGCGGCTCTAATCTGCCAGTGGCCACCC |
| | | | CTGACCCAGGCATGTTCCCCTGCCTGCACCACTCTCAGAACCTGCTGCG |
| | | | GGCCGTGAGCAATATGCTGCAGAAGGCCAGACAGACACTGGAGTTTTA |
| | | | CCCATGTGCCTCCAGAAAGAGCAGACACAAGGATATCACAAAGGATAA |
| | | | GACCTCTACAGTGGAGGCATGCCTGCCACTGGAGCTGACCAAGAACGA |
| | | | GAGCTGTCTGAACAGCCGGGAGACATCTTTCATCACCAACGGCAGCTGC |
| | | | CTGGCCTCCAGAAAGACATCTTTTATGATGGCCCTGTGCCTGTCTAGCA |
| | | | TCTACGAGGACCTGAAGATGTATCAGGTGGAGTTCAAGACCATGAACG |
| | | | CCAAGCTGCTGATGGACCCTAAGAGGCAGATCTTTCTGGATCAGAATAT |
| | | | GCTGGCCGTGATCGACGAGCTGATGCAGGCCCTGAACTTCAATAGCGA |
| | | | GACAGTGCCACAGAAGTCCTCTCTGGAGGAGCCCGATTTCTACAAGACC |
| | | | AAGATCAAGCTGTGCATCCTGCTGCACGCCTTCCGGATCAGAGCCGTGA |
| | | | CCATCGACCGCGTGATGAGCTACCTGAACGCCAGC |
| 336 | 24840 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGAGCCAGAGCTCCAGAGTGCTGGGCTCCGGCAAGACCCTGA |
| | | | CAATCCAGGTGAGCAGCTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACGACCTGAAGTACGA |
| | | | GAATTATACAAGCTCCTTCTTTATCAGGGACATCATCAAGCCCGATCCC |
| | | | CCTAAGAACCTGCAGCTGAAGCCTCTGAAGAATAGCCGCCAGGTGGAG |
| | | | GTGTCCTGGGAGTACCCTGACACCTGGAGCACACCACACTCCTATTTCT |
| | | | CTCTGACCTTTTGCGTGCAGGTGCAGGGCAAGTCTAAGAGGGAGAAGA |
| | | | AGGACCGCGTGTTCACCGATAAGACAAGCGCCACCGTGATCTGTCGGA |
| | | | AGAACGCCAGCATCTCCGTGCGGGCCCAGGATAGATACTATTCTAGCTC |
| | | | CTGGAGCGAGTGGGCCTCCGTGCCATGTTCTGGAGGAGGAGGCAGCGG |
| | | | CGGAGGAGGCTCCGGCGGCGGCGGCTCTAATCTGCCAGTGGCCACCCC |
| | | | TGACCCAGGCATGTTCCCCTGCCTGCACCACTCTCAGAACCTGCTGCGG |
| | | | GCCGTGAGCAATATGCTGCAGAAGGCCAGACAGACACTGGAGTTTTAC |
| | | | CCATGTACCTCCGAGGAGATCGACCACGAGGATATCACAAAGGATAAG |
| | | | ACCTCTACAGTGGAGGCATGCCTGCCACTGGAGCTGACCAAGAACGAG |
| | | | AGCTGTCTGAACAGCCGGGAGACATCTTTCATCACCAACGGCAGCTGCC |
| | | | TGGCCTCCAGAAAGACATCTTTTATGATGGCCCTGTGCCTGTCTAGCAT |
| | | | CTACGAGGACCTGAAGATGTATCAGGTGGAGTTCAAGACCATGAACGC |
| | | | CAAGCTGCTGATGGACCCTAAGAGGCAGATCTTTCTGGATCAGAATATG |
| | | | CTGGCCGTGATCGACGAGCTGATGCAGGCCCTGAACTTCAATAGCGAG |
| | | | ACAGTGCCACAGAAGTCCTCTCTGGAGGAGCCCGATTTCTACAAGACCA |
| | | | AGATCAAGCTGTGCATCCTGCTGCACGCCTTCCGGATCAGAGCCGTGAC |
| | | | CATCGACCGCGTGATGAGCTACCTGAACGCCAGC |
| 337 | 24841 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAGCAGCCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGCGGCGATAACAAGGAGTACGAG |
| | | | TATTCCGTGGAGTGCCAGGAGGACTCTGCCTGTCCCGCCGCCGAGGAGT |
| | | | CCCTGCCTATCGAAGTGATGGTGGATGCCGTGCACAAGCTGAAGTACG |
| | | | AGAATTATACAAGCTCCTTCTTTATCAGGGACATCATCAAGCCCGATCC |
| | | | CCCTAAGAACCTGCAGCTGAAGCCTCTGAAGAATAGCCGCCAGGTGGA |
| | | | GGTGTCCTGGGAGTACCCTGACACCTGGAGCACACCACACTCCTATTTC |
| | | | TCTCTGACCTTTTGCGTGCAGGTGCAGGGCAAGTCTAAGAGGGAGAAG |
| | | | AAGGACCGCGTGTTCACCGATAAGACAAGCGCCACCGTGATCTGTCGG |
| | | | AAGAACGCCAGCATCTCCGTGCGGGCCCAGGATAGATACTATTCTAGCT |
| | | | CCTGGAGCGAGTGGGCCTCCGTGCCATGTTCTGGAGGAGGAGGCAGCG |
| | | | GCGGAGGAGGCTCCGGCGGCGGCGGCTCTAATCTGCCAGTGGCCACCC |
| | | | CTGACCCAGGCATGTTCCCCTGCCTGCACCACTCTCAGAACCTGCTGCG |
| | | | GGCCGTGAGCAATATGCTGCAGAAGGCCAGACAGACACTGGAGTTTTA |
| | | | CCCATGTACCTCCGAGGAGATCGACCACGAGGATATCACAAAGGATAA |
| | | | GACCTCTACAGTGGAGGCATGCCTGCCACTGGAGCTGACCAAGAACGA |
| | | | GAGCTGTCTGAACAGCCGGGAGACATCTTTCATCACCAACGGCAGCTGC |
| | | | CTGGCCTCCAGAAAGACATCTTTTATGATGGCCCTGTGCCTGTCTAGCA |
| | | | TCTACGAGGACCTGAAGATGTATCAGGTGGAGTTCAAGACCATGAACG |
| | | | CCAAGCTGCTGATGGACCCTAAGAGGCAGATCTTTCTGGATCAGAATAT |
| | | | GCTGGCCGTGATCGACGAGCTGATGCAGGCCCTGAACTTCAATAGCGA |
| | | | GACAGTGCCACAGAAGTCCTCTCTGGAGGAGCCCGATTTCTACAAGACC |
| | | | AAGATCAAGCTGTGCATCCTGCTGCACGCCTTCCGGATCAGAGCCGTGA |
| | | | CCATCGACCGCGTGATGAGCTACCTGAACGCCAGC |
| 338 | 24842 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCTCAGGTGTACGTGTATCCACCCTCCCGCGATGAGCTGACAAAGAA |
| | | | CCAGGTGTCTCTGACCTGTCTGGTGAAGGGCTTTTACCCTAGCGACATC |
| | | | GCCGTGGAGTGGGAGTCCAATGGCCAGCCAGAGAACAATTATAAGACC |
| | | | ACACCTCCAGTGCTGGACTCTGATGGCAGCTTCGCCCTGGTGTCTAAGC |
| | | | TGACAGTGGACAAGAGCAGATGGCAGCAGGGCAACGTGTTTTCTTGTA |
| | | | GCGTGATGCACGAGGCCCTGCACAATCACTACACCCAGAAGTCCCTGA |
| | | | GCTTAAGCCCTGGAGGCAGCGCCGATGGAGGAATCTGGGAGCTGAAGA |
| | | | AGGACGTGTACGTGGTGGAGCTGGACTGGTATCCAGATGCACCAGGAG |
| | | | AGATGGTGGTGCTGACCTGCGACACACCAGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACCTGTCACA |
| | | | AGGGCGGCGAGGTGCTGTCCCACTCTCTGCTGCTGCTGCACAAGAAGG |
| | | | AGGACGGCATCTGGAGCACAGACATCCTGAAGGATCAGAAGGAGCCCA |
| | | | AGAACAAGACCTTCCTGAGGTGCGAGGCCAAGAATTATAGCGGCAGAT |
| | | | TCACCTGTTGGTGGCTGACCACAATCTCCACCGATCTGACATTTTCTGTG |
| | | | AAGTCTAGCAGGGGCTCCTCTGACCCTCAGGGAGTGACATGCGGAGCC |
| | | | GCCACCCTGAGCGCCGAGCGGGTGAGAGGCGATAACAAGGAGTACGAG |
| | | | TATTCCGTGGAGTGCCAGGAGGACTCTGCCTGTCCCGCCGCCGAGGAGT |
| | | | CCCTGCCTATCAGCGTGATGGTGGATGCCGTGCACAAGCTGAAGTACGA |
| | | | GAATTATAGCAGCAGATTCTTTATCAGGGACATCATCAAGCCCGATCCC |
| | | | CCTAAGAACCTGCAGCTGAAGCCTCTGAAGAATAGCCGCCAGGTGGAG |
| | | | GTGTCCTGGGAGTACCCTGACACCTGGAGCACACCACACTCCTATTTCT |
| | | | CTCTGACCTTTTGCGTGCAGGTGCAGGGCAAGTCTAAGAGGGAGAAGA |
| | | | AGGACCGCGTGTTCACCGATAAGACAAGCGCCACCGTGATCTGTCGGA |
| | | | AGAACGCCAGCATCTCCGTGCGGGCCCAGGATAGATACTATTCTAGCTC |
| | | | CTGGAGCGAGTGGGCCTCCGTGCCATGTTCTGGAGGAGGAGGCAGCGG |
| | | | CGGAGGAGGCTCCGGCGGCGGCGGCTCTAATCTGCCAGTGGCCACCCC |
| | | | TGACCCAGGCATGTTCCCCTGCCTGCACCACTCTCAGAACCTGCTGCGG |
| | | | GCCGTGAGCAATATGCTGCAGAAGGCCAGACAGACACTGGAGTTTTAC |
| | | | CCATGTACCTCCGAGGAGATCGACCACGAGGATATCACAAAGGATAAG |
| | | | ACCTCTACAGTGGAGGCATGCCTGCCACTGGAGCTGACCAAGAACGAG |
| | | | AGCTGTCTGAACAGCCGGGAGACATCTTTCATCACCAACGGCAGCTGCC |
| | | | TGGCCTCCAGAAAGACATCTTTTATGATGGCCCTGTGCCTGTCTAGCAT |
| | | | CTACGAGGACCTGAAGATGTATCAGGTGGAGTTCAAGACCATGAACGC |
| | | | CAAGCTGCTGATGGACCCTAAGAGGCAGATCTTTCTGGATCAGAATATG |
| | | | CTGGCCGTGATCGACGAGCTGATGCAGGCCCTGAACTTCAATAGCGAG |
| | | | ACAGTGCCACAGAAGTCCTCTCTGGAGGAGCCCGATTTCTACAAGACCA |
| | | | AGATCAAGCTGTGCATCCTGCTGCACGCCTTCCGGATCAGAGCCGTGAC |
| | | | CATCGACCGCGTGATGAGCTACCTGAACGCCAGC |
| 339 | 26498 | full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGAGGAGGCTCCGGCGGAGGAGGCTCCATGA |
| | | | GCGGGCGGAGCGCCAACGCAGGGGGCGGCGGCTCCGGCGGCGGCGGCT |
| | | | CTCAGGTGCAGCTGGTGGAGAGCGGAGGAGGAGTGGTGCAGCCCGGCA |
| | | | GAAGCCTGCGGCTGAGCTGCGCAGCCTCTGGCGTGACCTTTAGCTCCTA |
| | | | CGGCATGCACTGGGTGCGGCAGGCCCCTGGCAAGGGACTGGAGTGGGT |
| | | | GGCCTTCATCAGATATGACGGCAGCAATAAGTACTATGCCGATTCCGTG |
| | | | AAGGGCAGGTTTACCATCAGCCGCGACAACTCCAAGAATACACTGTAC |
| | | | CTGCAGATGAACAGCCTGAGGGCCGAGGACACCGCCGTGTACTATTGT |
| | | | AAGACACACGGCTCCCACGATAATTGGGGCCAGGGCACCATGGTGACA |
| | | | GTGTCTAGCGGCGGCTCTGGCGGCGGCAGCGGGGGTGGCTCTGGAGGA |
| | | | GGCAGCGGAGGAGGCTCCGGCCAGTCTGTGCTGACCCAGCCACCTTCTG |
| | | | TGAGCGGAGCCCCCGGACAGAGGGTGACAATCTCCTGCTCTGGCAGCC |
| | | | GCTCCAACATCGGCAGCAATACCGTGAAGTGGTATCAGCAGCTGCCAG |
| | | | GCACAGCCCCCAAGCTGCTGATCTACTATAATGACCAGCGGCCTTCCGG |
| | | | CGTGCCAGATAGATTCTCTGGCAGCAAGTCCGGCACCTCTGCCAGCCTG |
| | | | GCCATCACAGGCCTGCAGGCAGAGGACGAGGCAGATTACTATTGTCAG |
| | | | TCCTACGATAGATATACCCACCCCGCCCTGCTGTTTGGCACCGGCACAA |
| | | | AGGTGACAGTGCTG |
| 340 | 26320 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVYPPSRDELTKNQVSLT |
| | | | CLVKGFYPSDIAVEWESNGQPENNYKTTPPVLDSDGSFALVSKLTVDKSR |
| | | | WQQGNVFSCSVMHEALHNHYTQKSLSLSPGGSADGGIWELKKDVYVVEL |
| | | | DWYPDAPGEMVVLTCDTPEEDGITWTLDQSSEVLGSGKTLTIQVKEFGDA |
| | | | GQYTCHKGGEVLSHSLLLLHKKEDGIWSTDILKDQKEPKNKTFLRCEAKN |
| | | | YSGRFTCWWLTTISTDLTFSVKSSRGSSDPQGVTCGAATLSAERVRGDNKE |
| | | | YEYSVECQEDSACPAAEESLPIEVMVDAVHKLKYENYTSSFFIRDIIKPDPP |
| | | | KNLQLKPLKNSRQVEVSWEYPDTWSTPHSYFSLTFCVQVQGKSKREKKDR |
| | | | VFTDKTSATVICRKNASISVRAQDRYYSSSWSEWASVPCSGGGGSGGGGS |
| | | | GGGGSNLPVATPDPGMFPCLHHSQNLLRAVSNMLQKARQTLEFYPCTSEEI |
| | | | DHEDITKDKTSTVEACLPLELTKNESCLNSRETSFITNGSCLASRKTSFMMA |
| | | | LCLSSIYEDLKMYQVEFKTMNAKLLMDPKRQIFLDQNMLAVIDELMQALN |
| | | | FNSETVPQKSSLEEPDFYKTKIKLCILLHAFRIRAVTIDRVMSYLNASGGGG |
| | | | SGGGSGGGSGGGGSGGGGSGGGGSGGGGSEIVMTQSPATLSVSPGERATL |
| | | | SCRASQSISINLHWYQQKPGQAPRLLIYFASQSISGIPARFSGSGSGTEFTLTI |
| | | | SSLQSEDFAVYYCQQSNSFPLTFGGGTKVEIKGGSGGGSGGGSGGGSGGGS |
| | | | GQVQLVQSGAEVKKPGASVKVSCKASGYTFTDYYLHWVRQAPGQGLEW |
| | | | MGWIDPENGDTEYAPKFQGRVTMTTDTSTSTAYMELRSLRSDDTAVYYC |
| | | | NANKELRYFDVWGQGTMVTVSS |
| 341 | 26503 | full AA | EPKSSDKTHTCPPCPAPEAAGGPSVFLFPPKPKDTLMISRTPEVTCVVVSVS |
| | | | HEDPEVKFNWYVDGVEVHNAKTKPREEQYNSTYRVVSVLTVLHQDWLN |
| | | | GKEYKCKVSNKALPAPIEKTISKAKGQPREPQVYVLPPSRDELTKNQVSLL |
| | | | CLVKGFYPSDIAVEWESNGQPENNYLTWPPVLDSDGSFFLYSKLTVDKSR |
| | | | WQQGNVFSCSVMHEALHNHYTQKSLSLSPGGGGGSGGGGSGGGSGGGSG |
| | | | GGGSGGGGSQVQLVESGGGVVQPGRSLRLSCAASGFTFSSYGMHWVRQA |
| | | | PGKGLEWVAFIRYDGSNKYYADSVKGRFTISRDNSKNTLYLQMNSLRAED |
| | | | TAVYYCKTHGSHDNWGQGTMVTVSSGGSGGGSGGGSGGGSGGGSGQSV |
| | | | LTQPPSVSGAPGQRVTISCSGSRSNIGSNTVKWYQQLPGTAPKLLIYYNDQR |
| | | | PSGVPDRFSGSKSGTSASLAITGLQAEDEADYYCQSYDRYTHPALLFGTGT |
| | | | KVTVL |
| 342 | 26320 | full nt | GAGCCAAAGAGCTCCGACAAGACCCACACATGCCCCCCTTGTCCGGCG |
| | | | CCAGAGGCTGCAGGAGGACCAAGCGTGTTCCTGTTTCCACCCAAGCCTA |
| | | | AAGACACACTGATGATTTCCCGAACCCCCGAAGTCACATGCGTGGTCGT |
| | | | GTCTGTGAGTCACGAGGACCCTGAAGTCAAGTTCAACTGGTACGTGGAT |
| | | | GGCGTCGAGGTGCATAATGCCAAGACTAAACCTAGGGAGGAACAGTAC |
| | | | AACTCAACCTATCGCGTCGTGAGCGTCCTGACAGTGCTGCACCAGGATT |
| | | | GGCTGAACGGCAAAGAATATAAGTGCAAAGTGAGCAATAAGGCCCTGC |
| | | | CCGCTCCTATCGAGAAAACCATTTCCAAGGCTAAAGGGCAGCCTCGCG |
| | | | AACCACAGGTGTACGTGTATCCCCCTTCCCGGGACGAGCTGACCAAGA |
| | | | ACCAGGTGTCTCTGACATGCCTGGTGAAGGGCTTCTACCCCAGCGATAT |
| | | | CGCCGTGGAGTGGGAGTCCAATGGCCAGCCTGAGAACAATTATAAGAC |
| | | | CACACCACCCGTGCTGGACAGCGATGGCTCCTTCGCCCTGGTGTCCAAG |
| | | | CTGACCGTGGACAAGTCTAGGTGGCAGCAGGGCAACGTGTTTTCTTGCA |
| | | | GCGTGATGCACGAGGCCCTGCACAATCACTACACCCAGAAGTCCCTGA |
| | | | GCTTAAGCCCAGGAGGCAGCGCCGATGGAGGAATCTGGGAGCTGAAGA |
| | | | AGGACGTGTACGTGGTGGAGCTGGACTGGTACCCGGATGCCCCAGGCG |
| | | | AGATGGTGGTGCTGACCTGCGACACACCTGAGGAGGATGGCATCACCT |
| | | | GGACACTGGATCAGAGCTCCGAGGTGCTGGGCTCCGGCAAGACCCTGA |
| | | | CAATCCAGGTGAAGGAGTTCGGCGACGCCGGACAGTACACATGTCACA |
| | | | AGGGAGGAGAGGTGCTGAGCCACTCCCTGCTGCTGCTGCACAAGAAGG |
| | | | AGGACGGCATCTGGAGCACAGACATCCTGAAGGATCAGAAGGAGCCAA |
| | | | AGAACAAGACCTTCCTGCGGTGCGAGGCCAAGAATTATAGCGGCAGAT |
| | | | TCACCTGTTGGTGGCTGACCACAATCAGCACCGATCTGACATTTTCCGT |
| | | | GAAGTCTAGCAGGGGCTCCTCTGACCCCCAGGGAGTGACATGCGGAGC |
| | | | CGCCACCCTGAGCGCCGAGCGGGTGAGAGGCGATAACAAGGAGTACGA |
| | | | GTATTCCGTGGAGTGCCAGGAGGACTCTGCCTGTCCAGCCGCCGAGGA |
| | | | GTCCCTGCCAATCGAAGTGATGGTGGATGCCGTGCACAAGCTGAAGTA |
| | | | CGAGAATTATACAAGCTCCTTCTTTATCAGGGACATCATCAAGCCCGAT |
| | | | CCCCCTAAGAACCTGCAGCTGAAGCCCCTGAAGAACAGCAGACAGGTG |
| | | | GAGGTGAGCTGGGAGTACCCCGACACCTGGAGCACACCTCACTCCTATT |
| | | | TCTCTCTGACCTTTTGCGTGCAGGTGCAGGGCAAGTCTAAGAGGGAGAA |
| | | | GAAGGACCGCGTGTTCACCGATAAGACAAGCGCCACCGTGATCTGTAG |
| | | | AAAGAACGCCTCTATCAGCGTGCGGGCACAGGACCGGTACTACAGCTC |
| | | | CTCTTGGAGCGAGTGGGCCTCCGTGCCCTGCTCTGGCGGCGGCGGCTCT |
| | | | GGAGGAGGAGGCAGCGGCGGAGGAGGCTCCAACCTGCCTGTGGCCACC |
| | | | CCCGATCCTGGCATGTTCCCATGCCTGCACCACTCCCAGAACCTGCTGA |
| | | | GGGCCGTGTCTAATATGCTGCAGAAGGCCCGCCAGACACTGGAGTTTTA |
| | | | TCCCTGTACCTCCGAGGAGATCGACCACGAGGATATCACAAAGGACAA |
| | | | GACCAGCACAGTGGAGGCCTGCCTGCCTCTGGAGCTGACCAAGAACGA |
| | | | GAGCTGTCTGAACAGCCGGGAGACCAGCTTCATCACCAATGGCAGCTG |
| | | | CCTGGCCTCCAGAAAGACATCTTTTATGATGGCCCTGTGCCTGAGCTCC |
| | | | ATCTACGAGGATCTGAAGATGTATCAGGTGGAGTTCAAGACCATGAAC |
| | | | GCCAAGCTGCTGATGGACCCTAAGCGGCAGATCTTTCTGGATCAGAATA |
| | | | TGCTGGCCGTGATCGACGAGCTGATGCAGGCCCTGAACTTCAATTCCGA |
| | | | GACAGTGCCCCAGAAGTCTAGCCTGGAGGAGCCTGATTTCTACAAGAC |
| | | | CAAGATCAAGCTGTGCATCCTGCTGCACGCCTTTCGGATCAGAGCCGTG |
| | | | ACCATCGACAGAGTGATGTCTTATCTGAACGCCAGCGGCGGCGGAGGC |
| | | | TCTGGCGGCGGGAGCGGCGGCGGCAGCGGGGGAGGAGGCTCCGGAGG |
| | | | AGGAGGCTCTGGCGGCGGCGGCAGCGGCGGCGGCGGCAGCGAGATCGT |
| | | | GATGACACAGTCCCCTGCCACCCTGTCCGTGTCTCCAGGAGAGAGGGCC |
| | | | ACACTGAGCTGTAGAGCCAGCCAGTCCATCTCTATCAACCTGCACTGGT |
| | | | ATCAGCAGAAGCCAGGACAGGCCCCCAGGCTGCTGATCTATTTCGCCA |
| | | | GCCAGAGCATTTCTGGCATCCCTGCACGCTTCAGCGGCTCCGGCTCTGG |
| | | | CACCGAGTTTACCCTGACAATCTCCTCTCTGCAGTCCGAGGATTTTGCC |
| | | | GTGTACTATTGCCAGCAGAGCAATTCCTTCCCACTGACATTTGGCGGCG |
| | | | GCACCAAGGTGGAGATCAAGGGAGGCAGCGGCGGCGGCTCCGGCGGC |
| | | | GGCTCTGGCGGCGGCAGCGGAGGAGGCTCCGGACAGGTGCAGCTGGTG |
| | | | CAGAGCGGAGCCGAGGTGAAGAAGCCAGGGGCCAGCGTGAAGGTGAG |
| | | | CTGTAAGGCCTCCGGCTACACCTTCACAGACTACTATCTGCACTGGGTG |
| | | | AGGCAGGCCCCCGGCCAGGGACTGGAGTGGATGGGCTGGATCGACCCA |
| | | | GAGAACGGCGATACAGAGTACGCCCCCAAGTTTCAGGGCCGCGTGACC |
| | | | ATGACCACAGATACCTCTACAAGCACCGCCTATATGGAGCTGAGGTCCC |
| | | | TGCGCTCTGACGATACCGCCGTGTACTATTGTAACGCCAATAAGGAGCT |
| | | | GAGGTACTTTGACGTGTGGGGCCAGGGCACAATGGTGACCGTGAGCTC |
| | | | C |
| 343 | 26503 | full nt | GAGCCAAAGAGCTCCGACAAGACCCACACATGCCCCCCTTGTCCGGCG |
| | | | CCAGAGGCAGCAGGAGGACCAAGCGTGTTCCTGTTTCCACCCAAGCCC |
| | | | AAAGACACCCTGATGATTAGCCGAACCCCTGAAGTCACATGCGTGGTC |
| | | | GTGTCCGTGTCTCACGAGGACCCAGAAGTCAAGTTCAACTGGTACGTGG |
| | | | ATGGCGTCGAGGTGCATAATGCCAAGACAAAACCCCGGGAGGAACAGT |
| | | | ACAACAGCACCTATAGAGTCGTGTCCGTCCTGACAGTGCTGCACCAGGA |
| | | | TTGGCTGAACGGCAAGGAATATAAGTGCAAAGTGTCCAATAAGGCCCT |
| | | | GCCCGCTCCTATCGAGAAAACCATTTCTAAGGCAAAAGGCCAGCCTCGC |
| | | | GAGCCTCAGGTGTACGTGCTGCCACCTTCCCGCGACGAGCTGACCAAGA |
| | | | ATCAGGTGTCTCTGCTGTGCCTGGTGAAGGGCTTCTATCCAAGCGATAT |
| | | | CGCAGTGGAGTGGGAGTCCAACGGACAGCCCGAGAACAATTACCTGAC |
| | | | CTGGCCACCCGTGCTGGACAGCGATGGCTCCTTCTTTCTGTATTCCAAG |
| | | | CTGACAGTGGACAAGTCTAGATGGCAGCAGGGCAACGTGTTCAGCTGT |
| | | | TCCGTGATGCACGAGGCCCTGCACAACCACTACACCCAGAAGTCTCTGA |
| | | | GCTTAAGCCCTGGAGGAGGAGGAGGCTCCGGCGGAGGAGGCTCCGGCG |
| | | | GCGGGAGCGGCGGCGGCAGCGGGGGCGGCGGCTCCGGCGGCGGCGGC |
| | | | TCTCAGGTGCAGCTGGTGGAGAGCGGAGGAGGAGTGGTGCAGCCCGGC |
| | | | AGAAGCCTGCGGCTGAGCTGCGCAGCCTCTGGCTTCACCTTTAGCTCCT |
| | | | ACGGCATGCACTGGGTGCGGCAGGCCCCTGGCAAGGGACTGGAGTGGG |
| | | | TGGCCTTCATCAGATATGACGGCAGCAATAAGTACTATGCCGATTCCGT |
| | | | GAAGGGCAGGTTTACCATCAGCCGCGACAACTCCAAGAATACACTGTA |
| | | | CCTGCAGATGAACAGCCTGAGGGCCGAGGACACCGCCGTGTACTATTG |
| | | | TAAGACACACGGCTCCCACGATAATTGGGGCCAGGGCACCATGGTGAC |
| | | | AGTGTCTAGCGGCGGCTCTGGCGGCGGCAGCGGGGGTGGCTCTGGAGG |
| | | | AGGCAGCGGAGGAGGCTCCGGCCAGTCTGTGCTGACCCAGCCACCTTCT |
| | | | GTGAGCGGAGCCCCCGGACAGAGGGTGACAATCTCCTGCTCTGGCAGC |
| | | | CGCTCCAACATCGGCAGCAATACCGTGAAGTGGTATCAGCAGCTGCCA |
| | | | GGCACAGCCCCCAAGCTGCTGATCTACTATAATGACCAGCGGCCTTCCG |
| | | | GCGTGCCAGATAGATTCTCTGGCAGCAAGTCCGGCACCTCTGCCAGCCT |
| | | | GGCCATCACAGGCCTGCAGGCAGAGGACGAGGCAGATTACTATTGTCA |
| | | | GTCCTACGATAGATATACCCACCCCGCCCTGCTGTTTGGCACCGGCACA |
| | | | AAGGTGACAGTGCTG |
| 344 | | | AFTVTVPKDLYVVEYGSNMTIECKFPVEKQLDLAALQVF |
| | | | WMMEDKNIIQFVHGEEDLKVQHSSYRQRARLLKDQLSL |
| | | | GNAALQITDVKLQDAGVYTCLIAYKGADYKRITVKVNA |
| 345 | | | NPPTFSPALLVVTEGDNATFTCSFSNTSESFVLNWYRM |
| | | | SPSNQTDKLAAFPEDRSQPGQDSRFRVTQLPNGRDFHM |
| | | | SVVRARRNDSGTYLCGAISLAPKAQIKESLRAELRVTE |
| 346 | NA | PCS | GTGRSANA |
| 347 | NA | PCS | ASGRSANA |
| 348 | NA | PCS | GSGKSANA |
| 349 | NA | PCS | GSGRNAQV |
| 350 | NA | PCS | GSGKNAQV |
| 351 | NA | PCS | GTARLRGV |
| 352 | NA | PCS | GTSRMGTV |
| 353 | NA | PCS | GTSRQAQV |
| 354 | NA | PCS | AIKRSAQV |
| 355 | NA | PCS | STARMLQV |
| 356 | NA | PCS | GTQRSTGV |
| 357 | NA | PCS | GTRRDRIV |
| 358 | NA | PCS | GVARNYKV |
| 359 | NA | PCS | GGGRSANA |
| 360 | NA | PCS | GVGRSANA |
| 361 | NA | PCS | GIGRSANA |
| 362 | NA | PCS | GHGRSANA |
| 363 | NA | PCS | KSGRSANA |
| 364 | NA | PCS | TSGRSANA |
| 365 | NA | PCS | SSGRSANA |
| 366 | NA | PCS | RSGRSANA |

^{a}Protease cleavage sequence;
^{b}Briakinumab;
^{c}Modified IL12 heparin-binding loop sequence

TABLE 25
variants and their associated Seq ID’s

| Variant ID | Chain A Seq ID | Chain B Seq ID |
|---|---|---|
| 32862 | 306 | 62 |
| 35425 | 306 | 314 |
| 35456 | 306 | 81 |
| 36190 | 306 | 317 |
| 35436 | 341 | 62 |
| 35437 | 341 | 314 |
| 35457 | 341 | 340 |
| 36193 | 341 | 317 |
| 30806 | 20 | 62 |
| 33495 | 20 | 314 |
| 33498 | 20 | 317 |

### Example 22: Masked Cleavable IL12-Fc Variants Reduction in Tumor Growth in Mouse Tumors

In order to assess the ability of an engineered masked IL12-Fc to reduce tumor growth variants are tested in a humanized mouse tumor model. Immunodeficient mice are engrafted with a mixture of human tumor cells and PBMCs. Several weeks after engraftment mice are randomized into treatment groups and administered injections of either: vehicle control (VC); unmasked IL12-Fc (UM-IL12); masked non-cleavable IL12-Fc variant (MNC-IL12); or masked cleavable IL12-Fc variant (MC-IL12). Tumor growth in mice is monitored over a period of 60 days. Serum was isolated from peripheral blood collected for subsequent pharmacokinetic analysis of variants Tumors are collected at various timepoints after dosing and the concentration of intact and cleaved test article is quantified.

Mice dosed with VC and MNC-IL12 have profound and similar tumor growth over 60 days. Mice dosed with UM-IL12 and MC-IL12 have significant dose dependent reductions in tumor growth compared to VC and MNC-IL12. Tumor growth inhibition induced by UM-IL12 and MC-IL12 are similar.

Serum PK analysis shows prolonged serum exposure of MC-IL12. No cleaved MC-IL12 is detected in the serum at any timepoint. Cleaved MC-IL12 is detected in tumor samples at concentrations anticipated to agonize IL12 receptor. MNC-IL12 remains intact in all serum and tumor samples analyzed.

Collectively, MC-IL12 retains the anti-tumor activity of UM-IL12 and the activity of MC-IL12 is dependent on protease cleavage.

The various embodiments described herein can be combined to provide further embodiments. All U.S. patents, U.S. patent application publications, U.S. patent application, foreign patents, foreign patent applications, articles, books, manuals, treatises and other non-patent publications referred to in this specification and/or listed in the Application Data Sheet are incorporated herein by reference, in their entirety for the purpose of describing and disclosing, for example, the constructs and methodologies that are described in the publications, which might be used in connection with the methods, compositions and compounds described herein. Aspects of the embodiments can be modified if necessary to employ concepts of the various patents, applications, and publications to provide yet further embodiments. The publications discussed herein are provided solely for their disclosure prior to the filing date of the present application. Nothing herein is to be construed as an admission that the inventors described herein are not entitled to antedate such disclosure by virtue of prior invention or for any other reason.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A masked interleukin 12 (IL12) fusion protein, comprising:
a. an Fc domain comprising a first Fc polypeptide and a second Fc polypeptide;
b. a masking moiety (MM); and
c. an IL12 polypeptide;
wherein the masking moiety is fused to the first Fc polypeptide by a first linker; and
optionally, wherein the masking moiety further comprises a second linker;
wherein the IL12 polypeptide is fused to the second Fc polypeptide by a third linker;
wherein at least one of the first, second or third linkers is protease cleavable; and
wherein the IL12 activity of the masked IL12 fusion protein is attenuated as compared to the IL12 activity of the IL12 containing polypeptide released after cleavage of the at least one protease cleavable linker.

2. The masked IL12 fusion protein of claim 1, wherein the first linker is protease cleavable and optionally, the second linker is protease cleavable.

3. The masked IL12 fusion protein of claim 1, wherein the third linker is protease cleavable and optionally, the first or second linker is protease cleavable, or both.

4. The masked IL12 fusion protein of claim 1, wherein the first linker comprises a cleavage sequence selected from the group consisting of the cleavage sites listed in Table 3 and Table 24.

5. The masked IL12 fusion protein of claim 1, wherein the first linker comprises a cleavage sequence having the amino acid sequence MSGRSANA (SEQ ID NO:10).

6. The masked IL12 fusion protein of claim 1, wherein the protease cleavable linker is cleaved by a protease selected from the group consisting of a matrix metalloproteinase (MMP), a matriptase, a cathepsin, a kallikrein, a caspase, a serine protease, and an elastase.

7. The masked IL12 fusion protein of claim 1, wherein the first, second and third linkers are cleaved by the same protease.

8. The masked IL12 fusion protein of claim 1, wherein the masking moiety is a single-chain Fv (scFv) antibody fragment, an IL12 receptor β2 subunit (IL12Rβ2) or an IL12-binding fragment thereof, or an IL12 receptor β1 subunit (IL12Rβ1) or an IL12-binding fragment thereof.

9. The masked IL12 fusion protein of claim 8, wherein the scFv comprises the VHCDR1-3 having the amino acid sequences set forth in SEQ ID NOs:β-15, respectively and the VLCDR1-3 having the amino acid sequence set forth in SEQ ID NOs:16-18, respectively.

10. The masked IL12 fusion protein of claim 8, wherein the scFv comprises a VH and VL comprising the amino acid sequence set forth in SEQ ID NOs:11 and 12, respectively; or a VH and VL comprising the amino acid sequence set forth in SEQ ID NOs:255 and 256, respectively.

11. The masked IL12 fusion protein of claim 8, wherein the scFv comprises a variant of the VH having the amino acid sequence set forth in SEQ ID NO:11 wherein the variant is selected from the group consisting of H_Y32A; H_F27V; H_Y52AV; H_R52E; H_R52E_Y52AV; H_H95D; H_G96T; and H_H98A, according to Kabat numbering; and the VL having the amino acid sequence set forth in SEQ ID NO: 12.

12. The masked IL12 fusion protein of claim 8, wherein the masking moiety is selected from an ECD of human IL12Rβ2, amino acids 24-321 of human IL12Rβ2 (IL12Rβ224-321), amino acids 24-124 of human IL12Rβ2 (IL12Rβ24-124), amino acids 24-240 of human IL12Rβ1 (IL12Rβ124-240) and an IL23R ECD.

13. The masked IL12 fusion protein of claim 1, wherein the IL12 polypeptide comprises the amino acid sequence set forth in SEQ ID NO:22 or 23.

14. The masked IL12 fusion protein of claim 13, wherein the IL12 polypeptide comprises the p40 polypeptide amino acid sequence set forth in SEQ ID NO:22 and the p35 IL12 polypeptide is non-covalently bound to the p40 polypeptide.

15. The masked IL12 fusion protein of claim 13, wherein the IL12 polypeptide comprises the p35 polypeptide amino acid sequence set forth in SEQ ID NO:23 and the p40 IL12 polypeptide is non-covalently bound to the p40 polypeptide.

16. The masked IL12 fusion protein of claim 1, wherein the IL12 polypeptide is a single chain IL12 polypeptide selected from a single chain IL12 polypeptide having the orientation p35-linker-p40 or p40-linker-p35.

17. The masked IL12 fusion protein of claim 16, wherein the fusion protein is selected from variants 29243, 29244, 31277, 32039, 32042, 32045, and 32454.

18. The masked IL12 fusion protein of claim 16, wherein the single chain IL12 polypeptide is a p40-linker-p35 polypeptide that is fused to the second Fc polypeptide at the p40 polypeptide.

19. The masked IL12 fusion protein of claim 16, wherein the single chain IL12 polypeptide is a p35-linker-p40 polypeptide that is fused to the second Fc polypeptide at the p35 polypeptide.

20. The masked IL12 fusion protein of claim 18 or claim 19, wherein the single chain IL12 polypeptide is fused to the c-terminal end of the second Fc polypeptide.

21. The masked IL12 fusion protein of claim 18 or claim 19, wherein the single chain IL12 polypeptide is fused to the c-terminal end of the second Fc polypeptide and the masking moiety is fused to the c-terminal end of the first Fc polypeptide.

22. The masked IL12 fusion protein of claim 18 or claim 19, wherein the single chain IL12 polypeptide is fused to the second Fc polypeptide and wherein the third linker is protease cleavable.

23. The masked IL12 fusion protein of claim 18 or claim 19, wherein the P40 domain of the IL12 polypeptide has been modified to be more resistant to proteolytic cleavage as compared to an unmodified P40 domain.

24. The masked IL12 fusion protein of claim 20, wherein the masking moiety is a single-chain Fv (scFv) antibody fragment; and wherein the IL12 fusion protein further comprises a second masking moiety comprising an additional scFv fused by a fourth linker to the p35 domain of the IL12 polypeptide.

25. The masked IL12 fusion protein of claim 24, wherein the first and fourth linkers are protease cleavable.

26. The masked IL12 fusion protein of claim 20, wherein the masking moiety comprises a first scFv fused to a second scFv by a fourth linker.

27. The masked IL12 fusion protein of claim 26, wherein the first and fourth linkers are protease cleavable.

28. The masked IL12 fusion protein of claim 27, wherein the masking moiety is in the following orientation: first Fc polypeptide-L1-VH-VL-L4-VH-VL; or first Fc polypeptide-L1-VH-VL-L4-VL-VH.

29. The masked IL12 fusion protein of claim 28, wherein the first and fourth linkers are protease cleavable.

30. The masked IL12 fusion protein of claim 1, wherein the masking moiety comprises an IL12 receptor β2 subunit (IL12Rβ2) or an IL12-binding fragment thereof, and an IL12 receptor β1 subunit (IL12Rβ1) or an IL12-binding fragment thereof, fused by the second linker.

31. The masked IL12 fusion protein of claim 30, wherein the masking moiety comprises an IL12Rβ2-Ig domain fused to the c-terminal end of the first Fc polypeptide and the IL12Rβ1 fused by the second linker to the c-terminal end of the IL12Rβ2-Ig domain.

32. The masked IL12 fusion protein of claim 31, wherein the first and the second linker are protease cleavable.

33. The masked IL12 fusion protein of claim 20, wherein the masking moiety is an IL12Rβ1 or an IL12-binding fragment thereof; and wherein the IL12 fusion protein further comprises a second masking moiety comprising an IL12Rβ2 or an IL12-binding fragment thereof fused by a fourth linker to the p35 domain of the IL12 polypeptide.

34. The masked IL12 fusion protein of claim 33, wherein the first and the fourth linker are protease cleavable.

35. The masked IL12 fusion protein of claim 1 further comprising a targeting domain.

36. The masked IL12 fusion protein of claim 35 wherein the targeting domain specifically binds a tumor-associated antigen.

37. The masked IL12 fusion protein of claim 1, wherein the first Fc polypeptide comprises a first CH3 domain and the second Fc polypeptide comprises a second CH3 domain.

38. The masked IL12 fusion protein of claim 1, wherein the IL12 activity is determined by measuring relative cell abundance or cytokine production of a cell or a cell line that is sensitive to IL12.

39. The masked IL12 fusion protein of claim 38, wherein the cell or cell line is selected from PBMC, CD8+ T cells, a CTLL-2 cell line and an NK cell line.

40. The masked IL12 fusion protein of claim 38, wherein the IL12 activity is determined by measuring IFNγ release by CD8+ T cells.

41. The masked IL12 fusion protein of claim 38, wherein the IL12 activity is determined by measuring the relative cell abundance of NK cells.

42. The masked IL12 fusion protein of claim 36, wherein the first CH3 domain or the second CH3 domain or both comprise an asymmetric amino acid modification wherein the first and second CH3 domain preferentially pair to form a heterodimer rather than a homodimer.

43. A masked interleukin 12 (IL12) fusion protein, comprising:
a. an Fc domain comprising a first Fc polypeptide and a second Fc polypeptide;
b. a masking moiety (MM); and
c. an IL12 polypeptide;
wherein the masking moiety is fused to the first Fc polypeptide by a first linker; and
optionally, wherein the masking moiety further comprises a second linker;
wherein the IL12 polypeptide is fused to the second Fc polypeptide by a third linker;
optionally, wherein at least one of the first, second or third linkers is protease cleavable; and
wherein the IL12 activity of the masked IL12 fusion protein is attenuated as compared to the IL12 activity of a control IL12 polypeptide.

44. A masked IL12 fusion protein, comprising:
a. an Fc domain comprising a first Fc polypeptide and a second Fc polypeptide;
b. a first MM and a second MM; and
c. an IL12 polypeptide;
wherein the IL12 polypeptide comprises a p35 polypeptide and a p40 polypeptide; wherein the first MM is fused to the first Fc polypeptide by a first linker; wherein the p35 polypeptide is fused to the first MM by a second linker; wherein the second MM is fused to the second Fc polypeptide by a third linker; and wherein the p40 polypeptide is non-covalently bound to the p35 polypeptide; and
wherein at least one of the first, second or third linkers is protease cleavable; and
wherein the IL12 activity of the masked IL12 fusion protein is attenuated as compared to the IL12 activity of the IL12 containing polypeptide released after cleavage of the at least one protease cleavable linker.

45. A masked IL12 fusion protein, comprising:
a. an Fc domain comprising a first Fc polypeptide and a second Fc polypeptide;
b. a first MM and a second MM; and
c. an IL12 polypeptide;
wherein the IL12 polypeptide comprises a p35 polypeptide and a p40 polypeptide; wherein the p35 polypeptide is fused to the first Fc polypeptide by a first linker; wherein the first MM is fused to the p35 polypeptide by a second linker; wherein the second MM is fused to the second Fc polypeptide by a third linker; and wherein the p40 polypeptide is non-covalently bound to the p35 polypeptide; and
wherein at least one of the first, second or third linkers is protease cleavable; and
wherein the IL12 activity of the masked IL12 fusion protein is attenuated as compared to the IL12 activity of the IL12 containing polypeptide released after cleavage of the at least one protease cleavable linker.

46. The masked IL12 fusion protein of claim 43, wherein the first MM is fused to the C-terminal end of the first Fc polypeptide and wherein the second MM is fused to the C-terminal end of the second Fc polypeptide.

47. The masked IL12 fusion protein of claim 45, wherein the p35 polypeptide is fused to the N-terminal end of the first Fc polypeptide and wherein the second MM is fused to the N-terminal end of the second Fc polypeptide.

48. A composition comprising the masked IL12 fusion protein of any one of claims 1 to 47 and a pharmaceutically acceptable excipient.

49. An isolated nucleic acid encoding the masked IL12 fusion protein of any one of claims 1 to 47.

50. An expression vector comprising the isolated nucleic acid of claim 49.

51. A host cell comprising the isolated nucleic acid of claim 49 or the expression vector of claim 50.

52. A method of making a masked IL12 fusion protein comprising culturing the host cell of claim 51 under conditions suitable for expression of the masked IL12 fusion protein and optionally, recovering the masked IL12 fusion protein from the host cell culture medium.

53. A method of treating cancer in a subject comprising administering to the subject a therapeutically effective amount of the composition of claim 48.

54. A masked interleukin 23 (IL23) fusion protein, comprising:
a. an Fc domain comprising a first Fc polypeptide and a second Fc polypeptide;
b. a masking moiety;
c. a first protease cleavable linker; and
d. an IL23 polypeptide;
wherein the masking moiety is fused to the first Fc polypeptide by the first protease cleavable linker; and optionally, wherein the masking moiety further comprises a second protease cleavable linker;
wherein the IL23 polypeptide is fused to the second Fc polypeptide; and
wherein the IL23 activity of the masked IL23 fusion protein is attenuated as compared to the IL23 activity of the IL23 containing polypeptide released after cleavage of the protease cleavable linker.

55. The masked IL23 fusion protein of claim 54, wherein the IL23 is a single chain IL23 polypeptide selected from a single chain IL23 polypeptide having the orientation p19-linker-p40 or p40-linker-p19.

56. The masked IL23 fusion protein of claim 54, wherein the single chain IL23 polypeptide is a p40-linker-p19 polypeptide that is fused to the second Fc polypeptide at the p40 polypeptide.

57. The masked IL23 fusion protein of claim 54, wherein the single chain IL23 polypeptide is a p19-linker-p40 polypeptide that is fused to the second Fc polypeptide at the p19 polypeptide.

58. The masked IL23 fusion protein of claim 56 or claim 57, wherein the single chain IL23 polypeptide is fused to the c-terminal end of the second Fc polypeptide.

59. The masked IL23 fusion protein of claim 56 or claim 57, wherein the single chain IL23 polypeptide is fused to the c-terminal end of the second Fc polypeptide and the masking moiety is fused to the c-terminal end of the first Fc polypeptide.

60. A recombinant polypeptide comprising a protease cleavable linker (PCL) wherein the protease cleavable linker comprises the amino acid sequence MSGRSANA (SEQ ID NO:10).

61. The recombinant polypeptide of claim 60 comprising two heterologous polypeptides, a first polypeptide located amino (N) terminally to the PCL and a second polypeptide located carboxyl (C) terminally to the PCL.

62. The recombinant polypeptide of claim 61, wherein the two heterologous polypeptides are selected from a cytokine polypeptide, an antibody, an antigen-binding fragment of an antibody and an Fc domain.

63. The recombinant polypeptide of claim 61, wherein the recombinant polypeptide comprises a cytokine polypeptide, a MM, and an Fc domain.

64. The recombinant polypeptide of claim 63, wherein the MM is a single-chain Fv (scFv) antibody fragment that binds to the cytokine or a cytokine receptor polypeptide or a cytokine-binding fragment thereof.

65. The recombinant polypeptide of claim 61, wherein the recombinant polypeptide comprises an antibody or antigen binding fragment thereof that binds a target, and a MM that binds to the antibody or antigen binding fragment thereof and blocks binding of the antibody or antigen binding fragment thereof to the target.

66. An isolated polypeptide comprising a PCL, wherein the PCL comprises the amino acid sequence of SEQ ID NO:10, wherein the PCL is a substrate for a protease, wherein the isolated polypeptide comprises at least one moiety (M) selected from the group consisting of a moiety that is located amino (N) terminally to the PCL (MN), a moiety that is located carboxyl (C) terminally to the PCL (MC), and combinations thereof, and wherein the MN or MC is selected from the group consisting of an antibody or antigen binding fragment thereof; a cytokine or a functional fragment thereof; a MM; a cytokine receptor or a functional fragment thereof; an immunomodulatory receptor, or functional fragment thereof; an immune checkpoint protein or a functional fragment thereof; a tumor associated antigen; a targeting domain; a therapeutic agent; an antineoplastic agent; a toxic agent; a drug; and a detectable label.
